(19) United States
(12) Patent Application Publication
(10) Pub. No.: US 2009/0270361 A1
(43) Pub. Date: Oct. 29, 2009

(54) Substituted pyrazole derivatives and use thereof

(75) Inventors: Mitsuhiro Ito, Tsukuba-shi (JP); Naoki Tomita, Tsukuba-shi (JP); Tomohiro Kaku, Osaka (JP); Tomohiko Suzaki, Osaka (JP)

Correspondence Address: EDWARDS ANGELL PALMER & DODGE LLP, P.O. BOX 55874, BOSTON, MA, 02205, US

(73) Assignee: Takeda Pharmaceutical Company Limited, Osaka (JP)

(21) Appl. No.: 12/383,502

(22) Filed: Mar. 25, 2009

(30) Foreign Application Priority Data
Mar. 26, 2008 (JP) JP 082029/2008

(51) Int. Cl. A61K 31/4155; C07D 231/12; A61K 31/415; A61K 31/4439; C07D 401/12; C07D 403/10; A61P 35/00

(52) U.S. Cl. 514/210.18; 548/376.1; 514/406; 514/341; 546/275.4; 548/364.1

(57) ABSTRACT

The present invention aims to provide a novel pyrazole derivative and a pharmaceutical agent containing the same. The present invention provides a compound represented by the formula (I′)

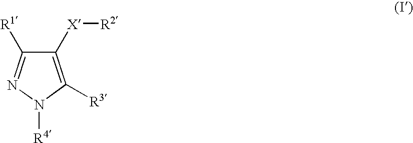

wherein
R¹′ is
(1) a hydrogen atom,
(2) a group via a carbon atom,
(3) a group via a nitrogen atom,
(4) a group via an oxygen atom or
(5) a group via a sulfur atom;
R²′ is an aromatic ring group optionally having substituent(s);
R³′ is
(1) a hydrogen atom,
(2) a group via a carbon atom,
(3) a group via a nitrogen atom,
(4) a group via an oxygen atom or
(5) a group via a sulfur atom;
R⁴′ is a cyanophenyl group optionally having substituent(s);
X′ is
(1) —Y′—CR⁵′R⁶′-Z′-
wherein R⁵′ and R⁶′ are the same or different and each is a hydrogen atom, a group via a carbon atom, a group via a nitrogen atom, a group via an oxygen atom or a group via a sulfur atom, or —CR⁵′R⁶′— is —C(alkylidene)-;
Y′ is a bond, —COCO—, —CONH—, —COCONH— or —O—; and
Z′ is a bond, —CH₂—, —CONH—, —O—, —OCH₂—, —S—, —SO—, —SO₂—, —CON(C₆H₅)— or

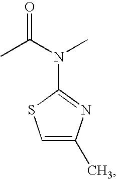

(2) —CO(CONH)ₙ— wherein n is 0 or 1,
(3) —NHCO—,
(4) —CONH—,
(5) —O—,
(6) —CH═CH— or
(7) —O(C₁₋₃ alkylene)O—; (excluding the compounds indicated to be excluded from the specification), or a salt thereof.

### TECHNICAL FIELD

The present invention relates to a novel pyrazole derivative and an androgen receptor antagonist containing the derivative, more particularly, the invention relates to a novel pyrazole derivative having a prophylactic. therapeutic effect on diseases dependent on androgen, which effect resulting from the inhibition of the receptor of androgen (AR), an androgenic hormone, and showing an androgen receptor antagonistic action free of an influence of mutation and the like, and an androgen receptor antagonist comprising the derivative.

### BACKGROUND OF INVENTION

Patent references 1 to 4 by the Applicant of the present invention disclose pyrrole derivatives having an androgen receptor binding inhibitory action.

In addition, patent documents 5-11 describe substituted pyrazole derivatives similar to the compound described in the specification of the present invention.
patent document 1: WO03/57669
patent document 2: WO2006/064944
patent document 3: JP-A-2007-332053
patent document 4: WO2007/145349
patent document 5: WO2006/111856
patent document 6: WO2005/058837
patent document 7: U.S. Pat. No. 5,807,863
patent document 8: U.S. Pat. No. 6,031,110
patent document 9: U.S. Pat. No. 5,298,527
patent document 10: JP-A-62-251753
patent document 11: Japanese patent application No. 2007-224910

### DISCLOSURE OF THE INVENTION

### Problems to be Solved by the Invention

The main object of the present invention is to provide a novel pyrazole derivative exhibiting a superior androgen receptor antagonistic action.

### Means of Solving the Problems

The present inventors have conducted various studies of pyrazole derivatives having an androgen receptor antagonistic action and found that the following novel pyrazole derivative unexpectedly shows a superior androgen receptor antagonistic action, superior pharmacokinetics and the like, or decreased toxicity, which resulted in the completion of the present invention.

That is, the present invention provides the following.
[1] A compound represented by the formula (I′)

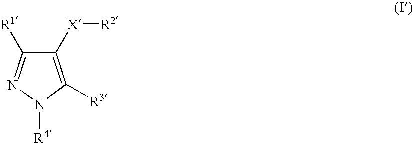

wherein
R¹′ is
(1) a hydrogen atom,
(2) a group via a carbon atom,
(3) a group via a nitrogen atom,
(4) a group via an oxygen atom or
(5) a group via a sulfur atom;
R²′ is an aromatic ring group optionally having substituent(s);
R³′ is
(1) a hydrogen atom,
(2) a group via a carbon atom,
(3) a group via a nitrogen atom,
(4) a group via an oxygen atom or
(5) a group via a sulfur atom;
R⁴′ is a cyanophenyl group optionally having substituent(s);
X′ is
(1) —Y′—CR⁵′R⁶′-Z′-
wherein R⁵′ and R⁶′ are the same or different and each is a hydrogen atom, a group via a carbon atom, a group via a nitrogen atom, a group via an oxygen atom or a group via a sulfur atom, or
—CR⁵′R⁶′— is —C(alkylidene)-;
Y′ is a bond, —COCO—, —CONH—, —COCONH— or —O—; and
Z′ is a bond, —CH₂—, —CONH—, —O—, —OCH₂—, —S—, —SO—, —SO₂—, —CON(C₆H₅)— or

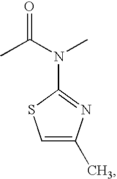

(2) —CO(CONH)ₙ— wherein n is 0 or 1,
(3) —NHCO—,
(4) —CONH—,
(5) —O—,
(6) —CH═CH— or
(7) —O(C₁₋₃ alkylene)O—;
excluding the following compounds:

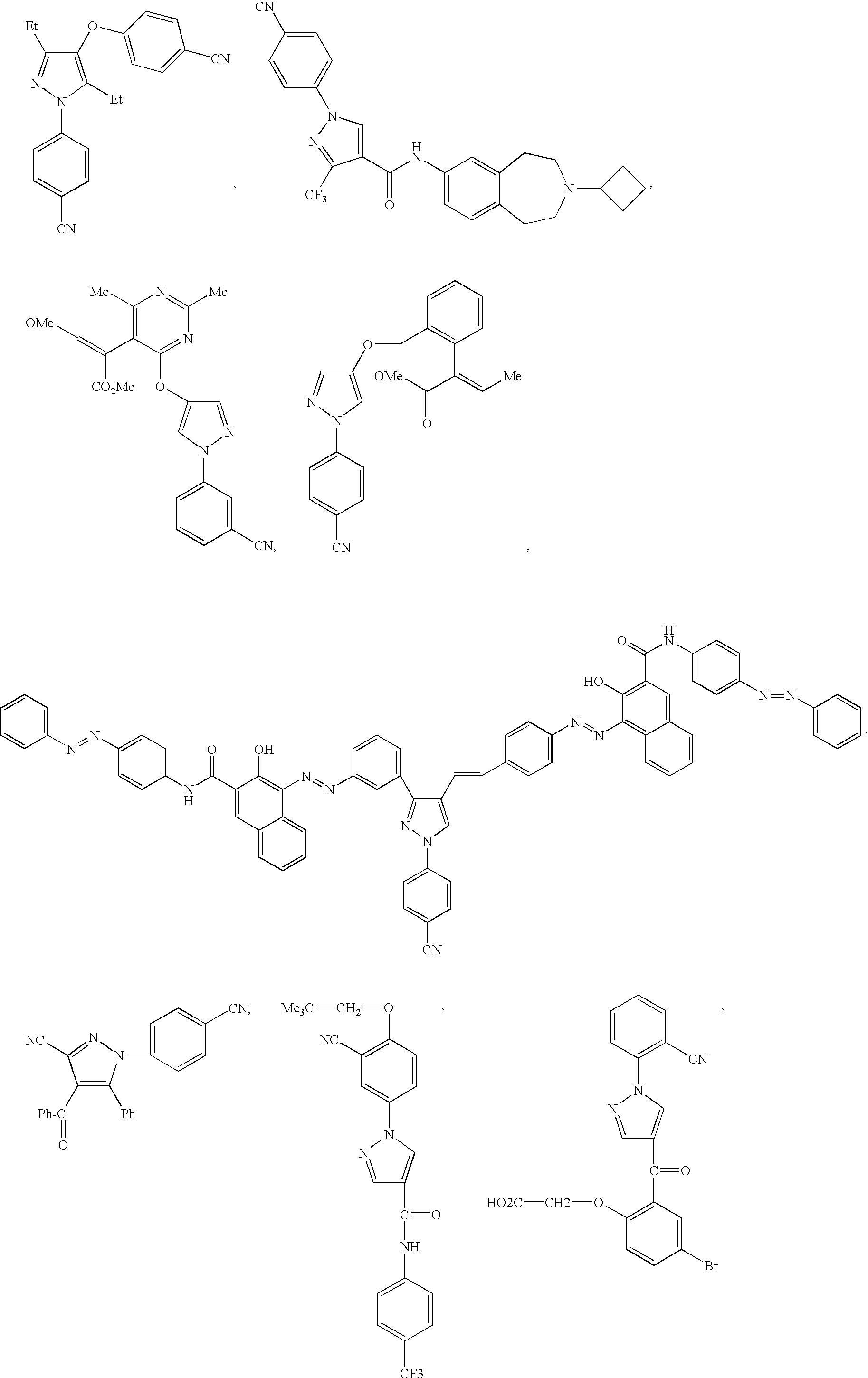

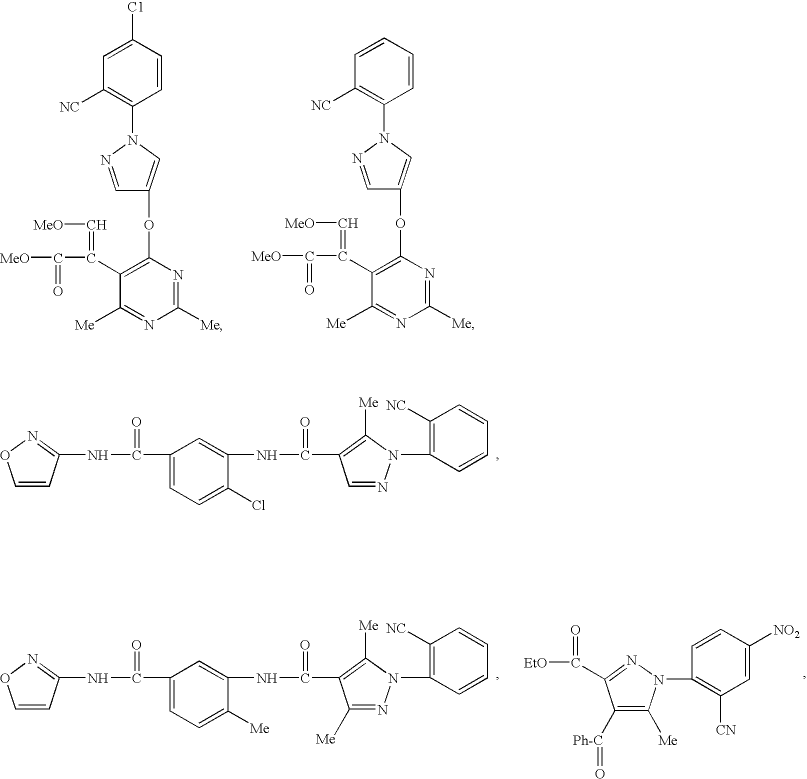

a compound represented by the formula:

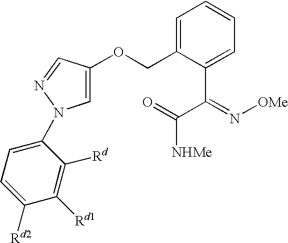

wherein one of R^{d}, R^{d1} and R^{d2} is a cyano group and the other two groups are the same or different and each is a hydrogen atom, a chlorine atom or a methyl group and
a compound represented by the formula:

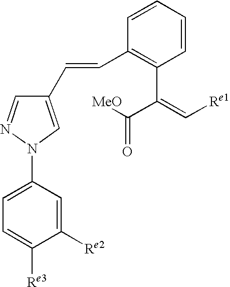

wherein R^{e1} is a methyl group, an ethyl group or a methoxymethyl group, one of R^{e2} and R^{e3} is a cyano group, and the other is a hydrogen atom,
or a salt thereof.
[2] A compound represented by the formula (I)

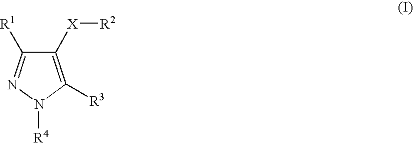

wherein
R¹ is
(1) a hydrogen atom,
(2) a group via a carbon atom,
(3) a group via a nitrogen atom,
(4) a group via an oxygen atom or
(5) a group via a sulfur atom;
R² is an aromatic ring group optionally having substituent(s);
R³ is
(1) a hydrogen atom,
(2) a group via a carbon atom,
(3) a group via a nitrogen atom,
(4) a group via an oxygen atom or
(5) a group via a sulfur atom;
R⁴ is a cyanophenyl group optionally having substituent(s);
X is
(1) —Y—CR⁵R⁶-Z-
wherein R⁵ and R⁶ are the same or different and each is a hydrogen atom, a group via a carbon atom, a group via a nitrogen atom, a group via an oxygen atom or a group via a sulfur atom, or
—CR⁵R⁶— is —C(alkylidene)-;
Y is a bond, —COCO—, —CONH—, —COCONH— or —O—; and
Z is a bond, —CH₂—, —CONH—, —O—, —OCH₂—, —S—, —SO— or —SO₂—,
(2) —CO(CONH)ₙ— wherein n is 0 or 1,
(3) —NHCO—,
(4) —CONH—,
(5) —O— or
(6) —CH═CH—;
excluding the following compounds:

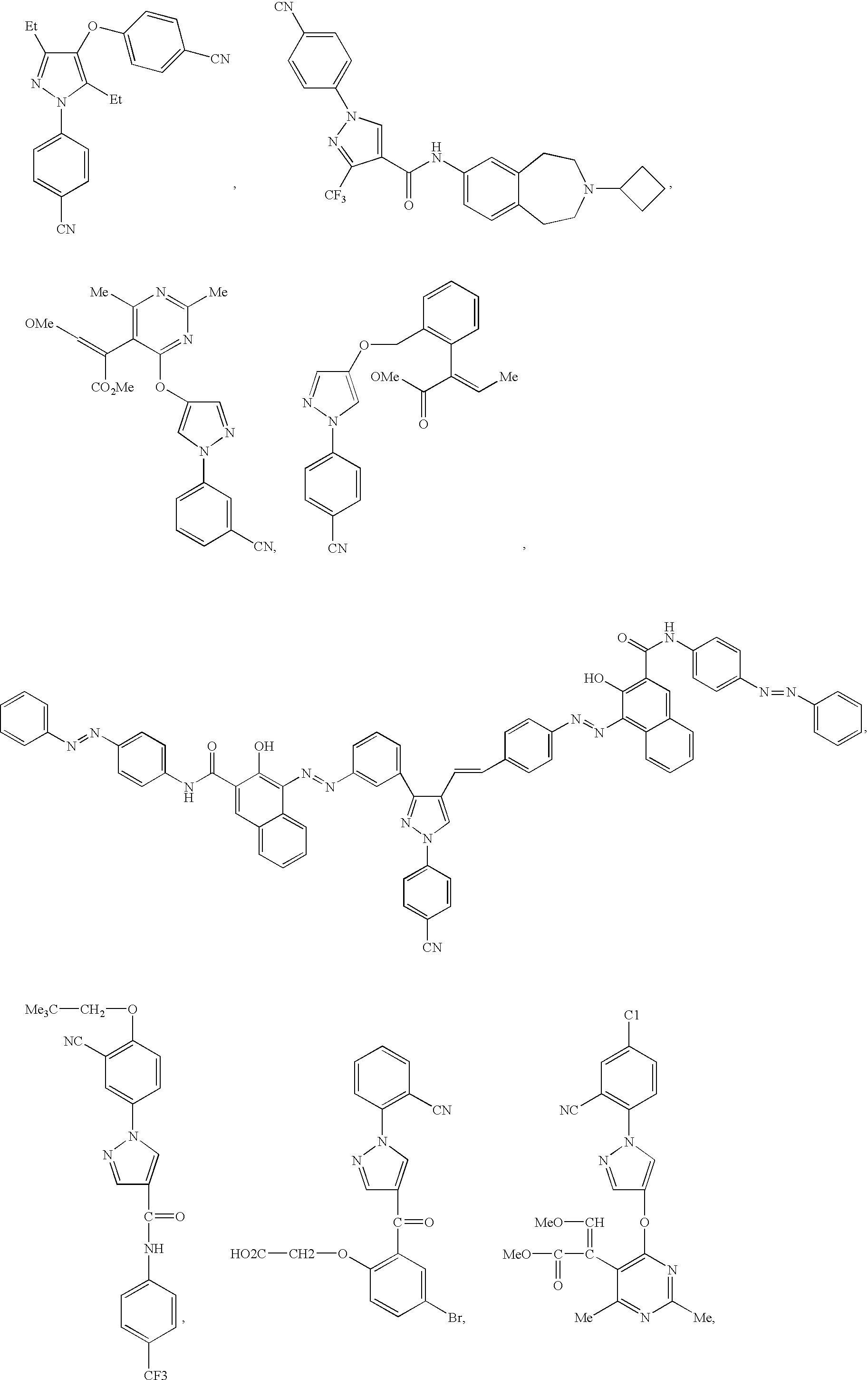

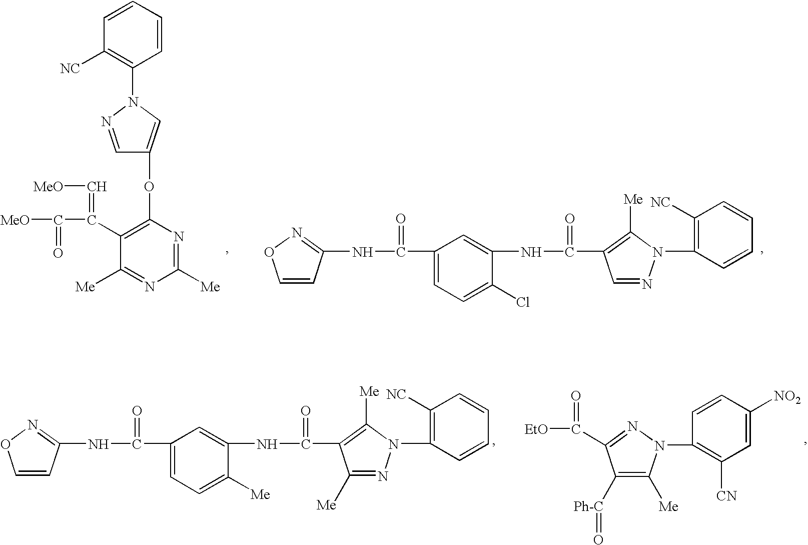

a compound represented by the formula:

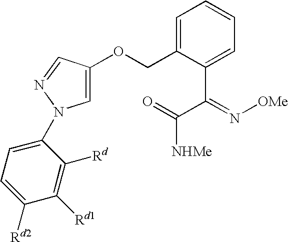

wherein one of R^{d}, R^{d1} and R^{d2} is a cyano group and the other two groups are the same or different and each is a hydrogen atom, a chlorine atom or a methyl group and
a compound represented by the formula:

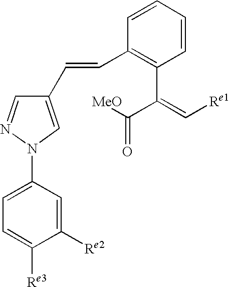

wherein R^{e1} is a methyl group, an ethyl group or a methoxymethyl group, one of R^{e2} and R^{e3} is a cyano group, and the other is a hydrogen atom,
or a salt thereof.
[3] The compound of the above-mentioned [2], wherein X is —CH₂—, —CH(CH₃)—, —CH(OH)—, —CH(OCOC₆H₅)—, —C(CH₃)(OH)—, —C(CH₂CH₃)(OH)—, —CO—, —C(═CH₂)—, —O—, —CH₂O—, —CH₂S—, —CH(CH₃)S—, —CH₂CH₂—, —CH₂SO₂—, —CH═CH—, —NHCO—, —CONH—, —C(CH₃)(OH)CH₂—, —OCH₂—, —COCONH—, —C(CH₃)(OH)CONH—, —CONHCH₂—, —CH₂OCH₂—, —COCOCH₂SO₂— or —COCONHCH₂—.
[4] The compound of the above-mentioned [2], wherein R¹ is an acyl group or a C₁₋₆ alkyl group optionally substituted by a hydroxy group.
[5] The compound of the above-mentioned [2], wherein R²is
(1) a phenyl group optionally having substituent(s),
(2) a pyridyl group optionally having substituent(s),
(3) a pyrazolyl group,
(4) an imidazolyl group,
(5) an oxazolyl group,
(6) a furyl group,
(7) a thienyl group,
(8) a thiazolyl group,
(9) an isoxazolyl group,
(10) a pyrimidinyl group,
(11) an indolyl group or
(12) a quinolyl group.
[6] The compound of the above-mentioned [2], wherein R³ is a C₁₋₆ alkyl-carbonyloxy group, an acyl group or a C₁₋₆ alkyl group optionally substituted by a hydroxy group.
[7] The compound of the above-mentioned [2], wherein R⁴ is a 4-cyanophenyl group optionally having substituent(s) selected from a cyano group, a halogen atom and a C₁₋₆ alkyl group optionally substituted by a halogen atom.
[8] The compound of the above-mentioned [2], wherein R¹ is an acyl group or a C₁₋₆ alkyl group optionally substituted by a hydroxy group;
R² is
(1) a phenyl group optionally having substituent(s),
(2) a pyridyl group optionally having substituent(s),
(3) a pyrazolyl group,
(4) an imidazolyl group,
(5) an oxazolyl group,
(6) a furyl group,
(7) a thienyl group,
(8) a thiazolyl group,
(9) an isoxazolyl group,
(10) a pyrimidinyl group,
(11) an indolyl group or
(12) a quinolyl group;
R³ is a C₁₋₆ alkyl-carbonyloxy group, an acyl group or a C₁₋₆ alkyl group optionally substituted by a hydroxy group;
R⁴ is a 4-cyanophenyl group optionally having substituent(s) selected from a cyano group, a halogen atom and a C₁₋₆ alkyl group optionally substituted by a halogen atom; and
X is —CH₂—, —CH(CH₃)—, —CH(OH)—, —CH(OCOC₆H₅)—, —C(CH₃)(OH)—, —C(CH₂CH₃)(OH)—, —CO—, —C(═CH₂)—, —O—, —CH₂O—, —CH₂S—, —CH(CH₃)S—, —CH₂CH₂—, —CH₂SO₂—, —CH═CH—, —NHCO—, —CONH—, —C(CH₃)(OH)CH₂—, —OCH₂—, —COCONH—, —C(CH₃)(OH)CONH—, —CONHCH₂—, —CH₂OCH₂—, —COCOCH₂SO₂— or —COCONHCH₂—.
[9] The compound of the above-mentioned [2], wherein
R¹ is a C₁₋₆ alkyl group optionally having substituent(s);
R²is a phenyl group optionally having substituent(s), a pyrazolyl group optionally having substituent(s), an imidazolyl group optionally having substituent(s), a furyl group optionally having substituent(s), an isoxazolyl group optionally having substituent(s), an oxazolyl group optionally having substituent(s), a pyridyl group optionally having substituent(s), a thienyl group optionally having substituent(s), a thiazolyl group optionally having substituent(s), a pyrimidinyl group optionally having substituent(s), an indolyl group optionally having substituent(s) or a quinolyl group optionally having substituent(s);
R³ is a C₁₋₆ alkyl group optionally having substituent(s), a C₁₋₆ alkyl-carbonyloxy group optionally having substituent(s) or a C₁₋₆ alkoxy-carbonyloxy group optionally having substituent(s);
R⁴ is a 4-cyanophenyl group optionally having substituent(s); and,
X is —CH₂—, —CH(CH₃)—, —CH(OH)—, —CH(OCOC₆H₅)—, —C(CH₃)(OH)—, —C(CH₂CH₃)(OH)—, —CO—, —C(═CH₂)—, —O—, —CH₂O—, —CH₂S—, —CH(CH₃)S—, —CH₂CH₂—, —CH₂SO₂—, —CH═CH—, —NHCO—, —CONH—, —C(CH₃)(OH)CH₂—, —OCH₂—, —COCONH—, —C(CH₃)(OH)CONH—, —CONHCH₂—, —CH₂OCH₂—, —COCOCH₂SO₂— or —COCONHCH₂—.
[10] A compound represented by the formula (Ia)

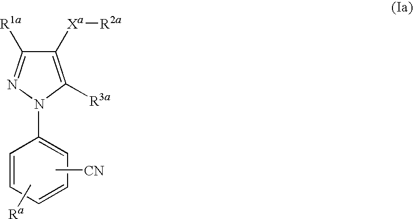

wherein
R^{a} is a hydrogen atom, a halogen atom, a cyano group or a C₁₋₆ alkyl group optionally having 1 to 3 halogen atoms;
X^{a} is C₁₋₃ alkylene, —O—, —O(C₁₋₃ alkylene)—, —CH(OH)—, —OCH(CH₃)—, —OCH(CONH₂)—, —O—CH₂CONH—, —CH(CH₃)—, —CH(OCOC₆H₅)—, —C(CH₃)(OH)—, —C(CH₂CH₃)(OH)—, —CO—, —C(═CH₂)—, —CH₂O—, —CH₂S—, —CH(CH₃)S—, —CH₂SO₂—, —CH═CH—, —NHCO—, —CONH—, —C(CH₃)(OH)CH₂—, —COCONH—, —C(CH₃)(OH)CONH—, —CONHCH₂—, —CH₂OCH₂—, —COCOCH₂SO₂—, —COCONHCH₂—, —C(CH₃)(OH)CH₂—, —O(C₁₋₃ alkylene)O—, —OCH₂CON(C₆H₅)— or

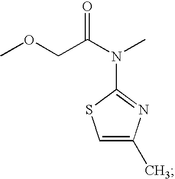

R^{1a} is an acyl group or a C₁₋₆ alkyl group optionally substituted by a hydroxy group;
R^{2a} is
(1) an aromatic hydrocarbon group optionally having substituent(s),
(2) a 5- to 7-membered mono-cyclic aromatic heterocyclic group containing 1 to 4 hetero atoms selected from an oxygen atom, a sulfur atom and a nitrogen atom, which optionally has substituent(s),
(3) a 8- to 12-membered condensed aromatic heterocyclic group containing 1 to 4 hetero atoms selected from an oxygen atom, a sulfur atom and a nitrogen atom, which optionally has substituent(s), or
(4) a nonaromatic heterocyclic group containing 1 to 4 hetero atoms selected from an oxygen atom, a sulfur atom and a nitrogen atom, which optionally has substituent(s); and
R^{3a} is a C₁₋₆ alkyl-carbonyloxy group, an acyl group or a C₁₋₆ alkyl group optionally substituted by a hydroxy group, provided that when X^{a} is —O—, then R^{1a} and R^{3a} are groups other than an ethyl group, and R^{2a} is a group other than a cyanophenyl group,
or a salt thereof.
[11] The compound of the above-mentioned [2], which is represented by the formula (Ib)

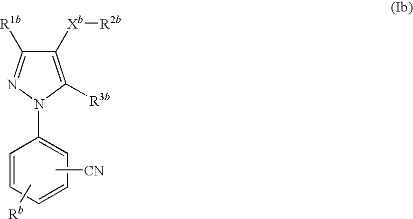

wherein
R is a hydrogen atom, a halogen atom, a cyano group or a C₁₋₆ alkyl group optionally having 1 to 3 halogen atoms;
X^{b} is —CH₂—, —C₂H₄—, —O—, —O(C₁₋₂ alkylene)—, —CH(OH)—, —OCH(CH₃)—, —OCH(CONH₂)—, —O—CH₂CONH—, —CH(CH₃)—, —CH(OCOC₆H₅)—, —C(CH₃)(OH)—, —C(CH₂CH₃)(OH)—, —CO—, —C(═CH₂)—, —CH₂O—, —CH₂S—, —CH(CH₃)S—, —CH₂SO₂—, —CH═CH—, —NHCO—, —CONH—, —C(CH₃)(OH)CH₂—, —O(CH₂)—, —COCONH—, —C(CH₃)(OH)CONH—, —CONHCH₂—, —CH₂OCH₂—, —COCOCH₂SO₂—, —COCONHCH₂— or —C(CH₃)(OH)CH₂—;
R^{1b} is an acyl group or a C₁₋₆ alkyl group optionally substituted by a hydroxy group;
R^{2b} is
(1) an aromatic hydrocarbon group optionally having substituent(s),
(2) a 5- to 7-membered mono-cyclic aromatic heterocyclic group containing 1 to 4 hetero atoms selected from an oxygen atom, a sulfur atom and a nitrogen atom, which optionally has substituent(s) or
(3) a 8- to 12-membered condensed aromatic heterocyclic group containing 1 to 4 hetero atoms selected from an oxygen atom, a sulfur atom and a nitrogen atom, which optionally has substituent(s);
R^{3b} is a C₁₋₆ alkyl-carbonyloxy group, an acyl group or a C₁₋₆ alkyl group optionally substituted by a hydroxy group, provided that when X^{b} is —O—, then R^{1b} and R^{3b} are groups other than an ethyl group.
[12] The compound of the above-mentioned [11], wherein R^{2b} is a phenyl group optionally having substituent(s), a pyridyl group optionally having substituent(s), a pyrimidinyl group optionally having substituent(s), a pyrazolyl group optionally having substituent(s), an imidazolyl group optionally having substituent(s), a thiazolyl group optionally having substituent(s), an oxazolyl group optionally having substituent(s), an oxadiazolyl group optionally having substituent(s), a pyrazolyl group optionally having substituent(s), a thienyl group optionally having substituent(s) or a thiazolyl group optionally having substituent(s).
[13] The compound of the above-mentioned [11], wherein R^{2b} is
(1) a phenyl group optionally having substituent(s) selected from an oxadiazolyl group optionally having substituent(s), a carbamoyl group optionally having substituent(s) and an acyl group or
(2) a pyridyl group optionally having substituent(s) selected from a C₁₋₆ alkyl-carbonylamino group and a carbamoyl group optionally having substituent(s).
[14] The compound of the above-mentioned [11], wherein
R^{b} is a halogen atom or a C₁₋₆ alkyl group optionally having 1 to 3 halogen atoms;
X^{b} is —CH₂—, —C₂H₄— or —O—;
R^{1b} and R^{3b} are each a C₁₋₆ alkyl group; and
R^{2b} is a phenyl group or pyridyl group optionally having substituent(s) selected from
(1) an oxadiazolyl group optionally having a carbamoyl group,
(2) a carbamoyl group substituted by a C₁₋₆ alkyl group optionally having a hydroxy group,
(3) a C₁₋₆ alkylcarbonyl-amino group,
(4) a dioxidothiomorpholinocarbonyl group,
(5) a morpholinocarbonyl group,
(6) a piperidinylcarbonyl group optionally having a hydroxy group and
(7) an azetidinylcarbonyl group optionally having a hydroxy group.
[15] The compound of the above-mentioned [2], which is represented by the formula (Ic)

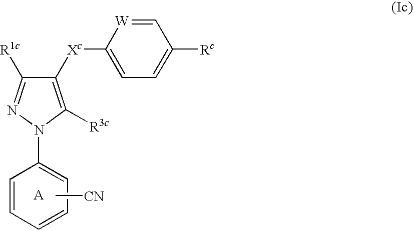

wherein
X^{c} is —CH₂—, —C₂H₄— or —O—;
R^{1c} and R^{3c} are the same or different and each is a C₁₋₆ alkyl group;
W is a nitrogen atom or CH;
ring A is a benzene ring further optionally having 1 to 3 halogen atoms; and
R^{c} is
(1) an oxadiazolyl group optionally having a carbamoyl group,
(2) a carbamoyl group optionally having a C₁₋₆ alkyl group optionally having a hydroxy group,
(3) a C₁₋₆ alkylcarbonyl-amino group,
(4) a dioxidothiomorpholinocarbonyl group,
(5) a morpholinocarbonyl group,
(6) a piperidinylcarbonyl group optionally having a hydroxy group or
(7) an azetidinylcarbonyl group optionally having a hydroxy group.
[16] The compound of the above-mentioned [2], which is represented by the formula (Id)

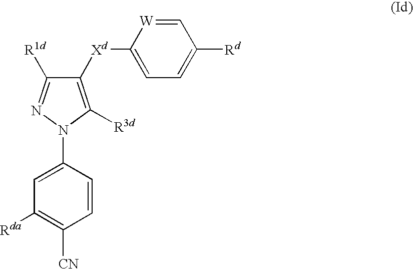

wherein
X^{d} is —O— or —CH₂—
W is a nitrogen atom or CH
R^{da} is a halogen atom,
R^{1d} and R^{3d} are each a methyl group, and
R^{d} is
(1) an oxadiazolyl group having a carbamoyl group,
(2) a C₁₋₆ alkyl-carbamoyl group optionally having a hydroxy group,
(3) a C₁₋₆ alkylcarbonyl-amino group,
(4) a dioxidothiomorpholinocarbonyl group,
(5) a morpholinocarbonyl group,
(6) a piperidinylcarbonyl group having a hydroxy group or
(7) an azetidinylcarbonyl group having a hydroxy group.
[17] 5-(4-{[1-(3-Chloro-4-cyanophenyl)-3,5-dimethyl-1H-pyrazol-4-yl]methyl}phenyl)-1,3,4-oxadiazole-2-carboxamide or a salt thereof.
[18] N-tert-Butyl-4-{[1-(3-chloro-4-cyanophenyl)-3,5-dimethyl-1H-pyrazol-4-yl]methyl}benzamide or a salt thereof.
[19] 2-Chloro-4-(4-{4-[(4-hydroxypiperidin-1-yl)carbonyl]benzyl}-3,5-dimethyl-1H-pyrazol-1-yl)benzonitrile or a salt thereof.
[20] 4-{[1-(3-Chloro-4-cyanophenyl)-3,5-dimethyl-1H-pyrazol-4-yl]methyl}-N-(2-hydroxy-2-methylpropyl)benzamide or a salt thereof.
[21] 2-Chloro-4-{3,5-dimethyl-4-[4-(morpholin-4-ylcarbonyl)phenoxy]-1H-pyrazol-1-yl}benzonitrile or a salt thereof.
[22] 2-Chloro-4-(4-{4-[(3-hydroxyazetidin-1-yl)carbonyl]benzyl}-3,5-dimethyl-1H-pyrazol-1-yl)benzonitrile or a salt thereof.
[23] 2-Chloro-4-(4-{4-[(1,1-dioxidothiomorpholin-4-yl)carbonyl]phenoxy}-3,5-dimethyl-1H-pyrazol-1-yl)benzonitrile or a salt thereof.
[24] N-(6-{[1-(3-Chloro-4-cyanophenyl)-3,5-dimethyl-1H-pyrazol-4-yl]oxy}pyridin-3-yl)-2,2-dimethylpropanamide or a salt thereof.
[25] A prodrug of the compound of the above-mentioned [1].
[26] A pharmaceutical agent comprising the compound of the above-mentioned [1] or a prodrug thereof.
[27] The pharmaceutical agent of the above-mentioned [26], which is an androgen receptor antagonist.
[28] The pharmaceutical agent of the above-mentioned [27], wherein the androgen receptor is a normal androgen receptor and/or a mutant androgen receptor.
[29] The pharmaceutical agent of the above-mentioned [26], which is an agent for the prophylaxis or treatment of hormone sensitive cancer in the androgen-dependent phase and/or the androgen-independent phase.
[30] The pharmaceutical agent of the above-mentioned [26], which is an agent for the prophylaxis or treatment of prostate cancer.
[31] A method of antagonizing an androgen receptor, comprising administering an effective amount of the compound of the above-mentioned [1] or a prodrug thereof to a mammal.
[32] A method of preventing or treating hormone sensitive cancer in the androgen-dependent phase and/or the androgen-independent phase, comprising administering an effective amount of the compound of the above-mentioned [1] or a prodrug thereof to a mammal.
[33] A method of preventing or treating prostate cancer, comprising administering an effective amount of the compound of the above-mentioned [1] or a prodrug thereof to a mammal.
[34] Use of the compound of the above-mentioned [1] or a prodrug thereof for the production of an androgen receptor antagonist.
[35] Use of the compound of the above-mentioned [1] or a prodrug thereof for the production of an agent for the prophylaxis or treatment of hormone sensitive cancer in the androgen-dependent phase and/or the androgen-independent phase.
[36] Use of the compound of the above-mentioned [1] or a prodrug thereof for the production of an agent for the prophylaxis or treatment of prostate cancer.

When compound (I′), compound (I) or compound (Ia) (hereinafter these compounds are to be referred to as the compound of the present invention), or a salt thereof contains an asymmetric carbon in the structure, all of the optically active forms and racemates are encompassed within the scope of the present invention. These compounds and salts thereof may be any of hydrates and anhydrates.

### EFFECT OF THE INVENTION

The compound of the present invention or a salt thereof shows not only strong antagonistic activity against natural androgen receptors, but also high antagonistic action on mutant androgen receptors. Moreover, these compounds are useful as, for example, pharmaceutical agents effective on prostate cancer in the hormone-independent phase, which can be administered orally, show extremely low toxicity, and have an androgen receptor antagonistic action.

### DETAILED DESCRIPTION OF THE INVENTION

In the present specification, the “halogen atom” means fluorine atom, chlorine atom, bromine atom, iodine atom and the like.

In the present specification, examples of the “group via a carbon atom” include
(1) cyano,
(2) alkyl (C₁₋₆ alkyl) optionally having substituent(s),
(3) alkenyl (C₂₋₆ alkenyl) optionally having substituent(s),
(4) alkynyl (C₂₋₆ alkynyl) optionally having substituent(s),
(5) cycloalkyl (C₃₋₈ cycloalkyl) optionally having substituent(s),
(6) cycloalkenyl (C₃₋₈ cycloalkenyl) optionally having substituent(s),
(7) aryl (C₆₋₁₀ aryl) optionally having substituent(s),
(8) acyl,
(9) a heterocyclic group optionally having substituent(s) (which has a bond at carbon atom), and the like.

Examples of the “C₁₋₆ alkyl” of the above-mentioned “C₁₋₆ alkyl optionally having substituent(s)” include methyl, ethyl, propyl, isopropyl, butyl, isobutyl, sec-butyl, tert-butyl, pentyl, hexyl and the like.

Examples of the “substituent” of the above-mentioned “C₁₋₆ alkyl optionally having substituent(s)” include substituent selected from the following substituent group A. While the number of substituents is not particularly limited as long as the substituents in the designated number are substitutable, it is preferably 1 to 5, more preferably 1 to 3.

Substituent group A: substituent group consisting of
(1) a halogen atom (e.g., fluorine atom, chlorine atom, bromine atom, iodine atom etc.);
(2) cyano;
(3) nitro;
(4) hydroxy;
(5) C₃₋₈ cycloalkyl (e.g., cyclopropyl, cyclobutyl, cyclopentyl, cyclohexyl, cycloheptyl, cyclooctyl etc.) optionally having 1 to 3 halogen atoms (e.g., fluorine);
(6) C₆₋₁₀ aryl (e.g., phenyl, 1-naphthyl, 2-naphthyl etc.) optionally having 1 to 3 substituents selected from a halogen atom and cyano;
(7) C₁₋₆ alkoxy (e.g., methoxy, ethoxy, propoxy, isopropoxy, butoxy, isobutoxy, tert-butoxy etc.) optionally having 1 to 3 substituents selected from a halogen atom, N-methyl-N-(2-pyrazyl)amino and 2-quinolyl;
(8) C₂₋₆ alkenyloxy (e.g., ethenyloxy, propenyloxy, butenyloxy, pentenyloxy, hexenyloxy etc.) optionally having 1 to 3 halogen atoms;
(9) C₂₋₆ alkynyloxy (e.g., ethynyloxy, propynyloxy, butynyloxy, pentynyloxy, hexynyloxy etc.) optionally having 1 to 3 halogen atoms;
(10) C₃₋₈ cycloalkyl-oxy (e.g., cyclopropyloxy, cyclobutyloxy, cyclopentyloxy, cyclohexyloxy etc.) optionally having 1 to 3 halogen atoms;
(11) C₃₋₈ cycloalkenyloxy (e.g., cyclopropenyloxy, cyclobutenyloxy, cyclopentenyloxy, cyclohexenyloxy etc.) optionally having 1 to 3 halogen atoms;
(12) C₆₋₁₀ aryloxy (e.g., phenyloxy, 1-naphthyloxy, 2-naphthyloxy etc.) optionally having 1 to 3 halogen atoms;
(13) C₃₋₈ cycloalkyl-C₁₋₆ alkoxy (e.g., cyclopropylmethyloxy, cyclopropylethyloxy, cyclobutylmethyloxy, cyclopentylmethyloxy, cyclohexylmethyloxy, cyclohexylethyloxy etc.) optionally having 1 to 3 halogen atoms;
(14) C₃₋₈ cycloalkenyl-C₁₋₆ alkoxy (e.g., cyclopentenylmethyloxy, cyclohexenylmethyloxy, cyclohexenylethyloxy, cyclohexenylpropyloxy etc.) optionally having 1 to 3 halogen atoms;
(15) C₆₋₁₀ aryl-C₁₋₆ alkoxy (e.g., phenylmethyloxy, phenylethyloxy etc.) optionally having 1 to 3 halogen atoms;
(16) C₁₋₆ alkyl-aminosulfonyl (e.g., methylaminosulfonyl, ethylaminosulfonyl, propylaminosulfonyl etc.);
(17) di-C₁₋₆ alkyl-aminosulfonyl (e.g., dimethylaminosulfonyl, diethylaminosulfonyl, dipropylaminosulfonyl etc.);
(18) carbamoyl;
(19) thiocarbamoyl;
(20) C₁₋₆ alkyl-carbamoyl (e.g., methylcarbamoyl, ethylcarbamoyl, propylcarbamoyl, isopropylcarbamoyl, trimethylpropylcarbamoyl, butylcarbamoyl, isobutylcarbamoyl, tert-butylcarbamoyl, N-tert-butyl-N-methylcarbamoyl etc.) optionally having 1 to 3 substituents selected from (a) a halogen atom, (b) hydroxy, (c) carboxy, (d) C₁₋₆ alkoxy (e.g., methoxy, ethoxy etc.) optionally having one hydroxy, (e) C₆₋₁₀ aryl (e.g., phenyl etc.), (f) 5- or 6-membered aromatic heterocyclic group (e.g., furyl, pyridyl etc.), (g) carbamoyl, (h) C₁₋₆ alkyl-carbamoyl (e.g., methylcarbamoyl), (i) di-C₁₋₆ alkyl-carbamoyl (e.g., dimethylcarbamoyl), (j) di-C₁₋₆ alkylamino (e.g., dimethylamino etc.), (k) C₃₋₆ cycloalkyl (e.g., cyclopropyl, cyclopentyl etc.) optionally having one hydroxy, (1) C₁₋₆ alkylthio (e.g., methylthio etc.), (m) C₁₋₆ alkylsulfinyl (e.g., methylsulfinyl etc.), (n) C₁₋₆ alkylsulfonyl (e.g., methylsulfonyl etc.), (o) C₁₋₆ alkoxy-carbonyl (e.g., methoxycarbonyl etc.), and (p) hydrazylcarbonyl optionally having one C₁₋₆ alkylcarbonyl (e.g., acetyl);
(21) C₃₋₆ cycloalkyl-carbamoyl (e.g., cyclopropylcarbamoyl, cyclopentylcarbamoyl etc.),
(22) di-C₁₋₆ alkyl-carbamoyl (e.g., dimethylcarbamoyl, diethylcarbamoyl, N-ethyl-N-methylcarbamoyl etc.) optionally having one substituent selected from C₁₋₆ alkoxy (e.g., methoxy etc.) and hydroxy,
(23) C₂₋₆ alkenyl-carbamoyl (e.g., 2-prop-2-en-1-yl-carbamoyl),
(24) C₆₋₁₀ aryl-carbamoyl (e.g., phenylcarbamoyl etc.) optionally having one C₁₋₆ alkyl (e.g., methyl etc.),
(25) 5- or 6-membered aromatic heterocycle (e.g., thiazolyl, pyridyl)-carbamoyl optionally having one C₁₋₆ alkyl (e.g., methyl etc.) optionally having 1 to 3 halogen atoms,
(26) cyclic carbamoyl (e.g., azetidinylcarbonyl, pyrrolidinocarbonyl, morpholinocarbonyl, thiomorpholinocarbonyl dioxide, piperazinocarbonyl etc.) optionally having 1 or 2 substituents selected from (a) hydroxy, (b) C₁₋₆ alkyl (e.g., methyl, ethyl etc.) optionally having one hydroxy, (c) carbamoyl, (d) di-C₁₋₆ alkylamino (e.g., dimethylamino etc.), (e) oxo and (f) C₆₋₁₀ aryl (e.g., phenyl etc.),
(27) di-C₁₋₆ alkyl-carbamoyl (e.g., dimethylcarbamoyl, diethylcarbamoyl, dipropylcarbamoyl etc.);
(28) hydrazylcarbonyl;
(29) formyl;
(30) C₁₋₆ alkyl-carbonyl (e.g., acetyl, ethylcarbonyl, propylcarbonyl, isopropylcarbonyl etc.);
(31) C₂₋₆ alkenyl-carbonyl (e.g., ethenylcarbonyl, propenylcarbonyl, butenylcarbonyl, pentenylcarbonyl, hexenylcarbonyl etc.);
(32) C₂₋₆ alkynyl-carbonyl (e.g., ethynylcarbonyl, propynylcarbonyl, butynylcarbonyl, pentynylcarbonyl, hexynylcarbonyl etc.);
(33) C₃₋₈ cycloalkyl-carbonyl (e.g., cyclopropylcarbonyl, cyclobutylcarbonyl, cyclopentylcarbonyl, cyclohexylcarbonyl etc.);
(34) C₃₋₈ cycloalkenyl-carbonyl (e.g., cyclopropenylcarbonyl, cyclobutenylcarbonyl, cyclopentenylcarbonyl, cyclohexenylcarbonyl etc.);
(35) C₆₋₁₀ aryl-carbonyl (e.g., benzoyl, 1-naphthylcarbonyl, 2-naphthylcarbonyl etc.);
(36) C₃₋₈ cycloalkyl-C₁₋₆ alkyl-carbonyl (e.g., cyclopropylmethylcarbonyl, cyclopropylethylcarbonyl, cyclobutylmethylcarbonyl, cyclopentylmethylcarbonyl, cyclohexylmethylcarbonyl, cyclohexylethylcarbonyl etc.);
(37) C₃₋₈ cycloalkenyl-C₁₋₆ alkyl-carbonyl (e.g., cyclopentenylmethylcarbonyl, cyclohexenylmethylcarbonyl, cyclohexenylethylcarbonyl, cyclohexenylpropylcarbonyl etc.);
(38) C₆₋₁₀ aryl-C₁₋₆ alkyl-carbonyl (e.g., benzylcarbonyl, phenylethylcarbonyl etc.);
(39) 5- or 6-membered mono-cyclic aromatic heterocyclyl-carbonyl (e.g., furylcarbonyl, thienylcarbonyl, pyrrolylcarbonyl, oxazolylcarbonyl, isoxazolylcarbonyl, thiazolylcarbonyl, isothiazolylcarbonyl, imidazolylcarbonyl, pyridylcarbonyl, pyrazolylcarbonyl etc.);
(40) 8- to 12-membered condensed aromatic heterocyclyl-carbonyl (e.g., benzofurylcarbonyl, isobenzofurylcarbonyl, benzothienylcarbonyl, isobenzothienylcarbonyl, indolylcarbonyl, isoindolylcarbonyl, 1H-indazolylcarbonyl, benzimidazolylcarbonyl, benzoxazolylcarbonyl etc.);
(41) 5- or 6-membered non-aromatic heterocyclyl-carbonyl (e.g., oxiranylcarbonyl, azetidinylcarbonyl, oxetanylcarbonyl, thietanylcarbonyl, pyrrolidinylcarbonyl, tetrahydrofurylcarbonyl, thioranylcarbonyl, piperidinylcarbonyl, morpholinylcarbonyl etc.);
(42) C₁₋₆ alkylsulfonyl (e.g., methylsulfonyl, ethylsulfonyl etc.);
(43) C₂₋₆ alkenylsulfonyl (e.g., ethenylsulfonyl, propenylsulfonyl etc.);
(44) C₂₋₆ alkynylsulfonyl (e.g., ethynylsulfonyl, propynylsulfonyl, butynylsulfonyl, pentynylsulfonyl, hexynylsulfonyl etc.);
(45) C₃₋₈ cycloalkylsulfonyl (e.g., cyclopropylsulfonyl, cyclobutylsulfonyl etc.);
(46) C₃₋₈ cycloalkenylsulfonyl (e.g., cyclopropenylsulfonyl, cyclobutenylsulfonyl etc.);
(47) C₆₋₁₀ arylsulfonyl (e.g., phenylsulfonyl etc.);
(48) C₃₋₈ cycloalkyl-C₁₋₆ alkyl-sulfonyl (e.g., cyclopropylmethylsulfonyl etc.);
(49) C₃₋₈ cycloalkenyl-C₁₋₆ alkyl-sulfonyl (e.g., cyclopentenylmethylsulfonyl etc.);
(50) C₆₋₁₀ aryl-C₁₋₆ alkyl-sulfonyl (e.g., benzylsulfonyl etc.);
(51) 5- or 6-membered mono-cyclic aromatic heterocyclyl-sulfonyl (e.g., furylsulfonyl, thienylsulfonyl, pyridylsulfonyl etc.);
(52) 8- to 12-membered condensed aromatic heterocyclyl-sulfonyl (e.g., benzofurylsulfonyl, isobenzofurylsulfonyl etc.);
(53) 5- or 6-membered nonaromatic heterocyclyl-sulfonyl (e.g., oxiranylsulfonyl, azetidinylsulfonyl etc.);
(54) amino;
(55) mono-C₁₋₆ alkylamino (e.g., methylamino, ethylamino, propylamino, isopropylamino, butylamino, isobutylamino, tert-butylamino etc.);
(56) di-C₁₋₆ alkylamino (e.g., dimethylamino, diethylamino, dipropylamino, diisopropylamino, dibutylamino, diisobutylamino, di-tert-butylamino etc.);
(57) C₁₋₆ alkoxy-carbonylamino (e.g., tert-butoxycarbonylamino etc.)
(58) mono-(C₁₋₆ alkyl-carbonyl)amino (e.g., acetylamino, ethylcarbonylamino, propylcarbonylamino, tert-butylcarbonylamino etc.) optionally having 1 to 3 halogen atoms;
(59) mono-(C₃₋₈ cycloalkyl-carbonyl)amino (e.g., cyclopropylcarbonylamino, cyclobutylcarbonylamino, cyclopentylcarbonylamino, cyclohexylcarbonylamino etc.);
(60) mono-(C₆₋₁₀ aryl-carbonyl)amino (e.g., benzoylamino etc.) optionally having 1 to 3 halogen atoms;
(61) C₁₋₆ alkyl-sulfonylamino (e.g., methanesulfonylamino);
(62) mono-(5- or 6-membered mono-cyclic aromatic heterocyclyl-carbonyl)amino (e.g., furylcarbonylamino, thienylcarbonylamino, pyrrolylcarbonylamino, oxazolylcarbonylamino, isoxazolylcarbonylamino, thiazolylcarbonylamino, isothiazolylcarbonylamino, imidazolylcarbonylamino, pyridylcarbonylamino, pyrazolylcarbonylamino etc.);
(63) mono-(8- to 12-membered condensed aromatic heterocyclyl-carbonyl)amino (e.g., benzofurylcarbonylamino, isobenzofurylcarbonylamino, benzothienylcarbonylamino, isobenzothienylcarbonylamino etc.);
(64) mono-(5- or 6-membered non-aromatic heterocyclyl-carbonyl)amino (e.g., oxiranylcarbonylamino, azetidinylcarbonylamino, oxetanylcarbonylamino etc.);
(65) mercapto;
(66) C₁₋₆ alkylthio (e.g., methylthio, ethylthio etc.) optionally having 1 to 3 halogen atoms;
(67) C₂₋₆ alkenylthio (e.g., ethenylthio, propenylthio etc.);
(68) C₂₋₆ alkynylthio (e.g., ethynylthio, propynylthio, butynylthio, pentynylthio, hexynylthio etc.);
(69) C₃₋₈ cycloalkylthio (e.g., cyclopropylthio, cyclobutylthio etc.);
(70) C₃₋₈ cycloalkenylthio (e.g., cyclopropenylthio, cyclobutenylthio etc.);
(71) C₆₋₁₀ arylthio (e.g., phenylthio etc.);
(72) C₃₋₈ cycloalkyl-C₁₋₆ alkyl-thio (e.g., cyclopropylmethylthio etc.);
(73) C₃₋₈ cycloalkenyl-C₁₋₆ alkyl-thio (e.g., cyclopentenylmethylthio etc.);
(74) a 5- or 6-membered mono-cyclic aromatic heterocyclic group (e.g., furyl, thienyl, pyrrolyl, oxazolyl, isoxazolyl, thiazolyl, isothiazolyl, imidazolyl, pyridyl, pyrazolyl, triazolyl, oxadiazolyl, thiadiazolyl etc.) optionally having one substituent selected from (a) hydroxy, (b) C₁₋₆ alkyl (e.g., methyl, propyl, isopropyl, tert-butyl etc.) optionally having 1 to 3 substituents selected from (i) halogen atom (e.g., fluorine atom etc.), (ii) hydroxy, (iii) C₁₋₆ alkoxy, (iv) di-C₁₋₆ alkylamino (e.g., dimethylamino), (v) C₁₋₆ alkoxycarbonyl (e.g., methoxycarbonyl) and (vi) C₁₋₆ alkylsulfonyl (e.g., methanesulfonyl), (c) C₃₋₆ cycloalkyl (e.g., cyclopropyl, cyclopentyl etc.), (d) cyano, (e) carbamoyl, (f) C₁₋₆ alkyl-carbamoyl (e.g., methylcarbamoyl, ethylcarbamoyl) optionally having one hydroxy, (g) di-C₁₋₆ alkyl-carbamoyl (dimethylcarbamoyl), (h) C₁₋₆ alkoxy-carbonyl (methoxycarbonyl) and (i) C₁₋₆ alkyl-carbonyl (acetyl);
(75) 8- to 12-membered condensed aromatic heterocyclic group (e.g., benzofuryl, isobenzofuryl, benzothienyl, isobenzothienyl, indolyl, isoindolyl, 1H-indazolyl, benzimidazolyl, benzoxazolyl, imidazoxadiazolyl etc.);
(76) 5- or 6-membered nonaromatic heterocyclic group (e.g., oxiranyl, azetidinyl, oxetanyl, thietanyl, pyrrolidinyl, tetrahydrofuryl, thioranyl, piperidinyl, morpholinyl etc.) optionally having oxo;
(77) 5- or 6-membered mono-cyclic aromatic heterocyclyl-oxy (e.g., furyloxy, thienyloxy, pyrrolyloxy, oxazolyloxy, isoxazolyloxy, thiazolyloxy, isothiazolyloxy, imidazolyloxy, pyridyloxy, pyrazolyloxy etc.);
(78) 8- to 12-membered condensed aromatic heterocyclyl-oxy (e.g., benzofuryloxy, isobenzofuryloxy, benzothienyloxy, isobenzothienyloxy, indolyloxy, isoindolyloxy, 1H-indazolyloxy, benzimidazolyloxy, benzoxazolyloxy etc.);
(79) 5- or 6-membered non-aromatic heterocyclyl-oxy (e.g., oxiranyloxy, azetidinyloxy, oxetanyloxy, thietanyloxy, pyrrolidinyloxy, tetrahydrofuryloxy, thioranyloxy, piperidinyloxy etc.);
(80) oxo;
(81) C₁₋₆ alkylsulfinyl (e.g., methylsulfinyl, ethylsulfinyl etc.);
(82) C₂₋₆ alkenylsulfinyl (e.g., ethenylsulfinyl, propenylsulfinyl etc.);
(83) C₂₋₆ alkynylsulfinyl (e.g., ethynylsulfinyl, propynylsulfinyl, butynylsulfinyl, pentynylsulfinyl, hexynylsulfinyl etc.);
(84) C₃₋₈ cycloalkylsulfinyl (e.g., cyclopropylsulfinyl, cyclobutylsulfinyl etc.);
(85) C₃₋₈ cycloalkenylsulfinyl (e.g., cyclopropenylsulfinyl, cyclobutenylsulfinyl etc.);
(86) C₆₋₁₀ arylsulfinyl (e.g., phenylsulfinyl etc.);
(87) C₃₋₈ cycloalkyl-C₁₋₆ alkyl-sulfinyl (e.g., cyclopropylmethylsulfinyl etc.);
(88) C₃₋₈ cycloalkenyl-C₁₋₆ alkyl-sulfinyl (e.g., cyclopentenylmethylsulfinyl etc.);
(89) C₁₋₆ alkyl-aminothiocarbonyl (e.g., methylaminothiocarbonyl, ethylaminothiocarbonyl, propylaminothiocarbonyl etc.);
(90) di-C₁₋₆ alkyl-aminothiocarbonyl (e.g., dimethylaminothiocarbonyl, diethylaminothiocarbonyl, dipropylaminothiocarbonyl etc.);
(91) carboxy;
(92) C₁₋₆ alkoxy-carbonyl (e.g., methoxycarbonyl, ethoxycarbonyl, propoxycarbonyl, isopropoxycarbonyl, butoxycarbonyl, isobutoxycarbonyl, tert-butoxycarbonyl etc.);
(93) C₂₋₆ alkenyloxy-carbonyl (e.g., ethenyloxycarbonyl, propenyloxycarbonyl, butenyloxycarbonyl, pentenyloxycarbonyl, hexenyloxycarbonyl etc.);
(94) C₂₋₆ alkynyloxy-carbonyl (e.g., ethynyloxycarbonyl, propynyloxycarbonyl, butynyloxycarbonyl, pentynyloxycarbonyl, hexynyloxycarbonyl etc.);
(95) C₃₋₈ cycloalkyl-oxy-carbonyl (e.g., cyclopropyloxycarbonyl, cyclobutyloxycarbonyl, cyclopentyloxycarbonyl, cyclohexyloxycarbonyl etc.);
(96) C₃₋₈ cycloalkenyloxy-carbonyl (e.g., cyclopropenyloxycarbonyl, cyclobutenyloxycarbonyl, cyclopentenyloxycarbonyl, cyclohexenyloxycarbonyl etc.);
(97) C₆₋₁₀ aryloxy-carbonyl (e.g., phenyloxycarbonyl, 1-naphthyloxycarbonyl, 2-naphthyloxycarbonyl etc.);
(98) C₃₋₈ cycloalkyl-C₁₋₆ alkoxy-carbonyl (e.g., cyclopropylmethyloxycarbonyl, cyclopropylethyloxycarbonyl, cyclobutylmethyloxycarbonyl, cyclopentylmethyloxycarbonyl, cyclohexylmethyloxycarbonyl, cyclohexylethyloxycarbonyl etc.);
(99) C₃₋₈ cycloalkenyl-C₁₋₆ alkoxy-carbonyl (e.g., cyclopentenylmethyloxycarbonyl, cyclohexenylmethyloxycarbonyl, cyclohexenylethyloxycarbonyl, cyclohexenylpropyloxycarbonyl etc.);
(100) C₆₋₁₀ aryl-C₁₋₆ alkoxy-carbonyl (e.g., phenylmethyloxycarbonyl, phenylethyloxycarbonyl etc.);
(101) aromatic heterocyclic group (e.g., triazinyl etc.)-amino-carbamoyl optionally having two C₁₋₆ alkoxy (e.g., methoxy etc.);
(102) C₂₋₃ alkyleneoxy (e.g., ethyleneoxy etc.) optionally having 1 or 2 substituents selected from C₁₋₆ alkyl (e.g., methyl etc.) and oxo,
(103) C₁₋₂ alkylenedioxy (e.g., methylenedioxy etc.);
(104) C₂₋₄ alkylene (e.g., trimethylene, tetramethylene etc.) optionally having oxo; and,
(105) C₂₋₄ alkenyleneoxy (e.g., propenyleneoxy etc.) optionally having 1 or 2 substituents selected from C₁₋₆ alkyl (e.g., methyl etc.) and oxo.

Examples of the “C₂₋₆ alkenyl” of the above-mentioned “C₂₋₆ alkenyl optionally having substituent(s)” include ethenyl, propenyl, butenyl, pentenyl, hexenyl and the like.

Examples of the “substituent” of the above-mentioned “C₂₋₆ alkenyl optionally having substituent(s)” include substituent selected from substituent group A. While the number of substituents is not particularly limited as long as the substituents in the designated number are substitutable, it is preferably 1 to 5, more preferably 1 to 3.

Examples of the “C₂₋₆ alkynyl” of the above-mentioned “C₂₋₆ alkynyl optionally having substituent(s)” include ethynyl, propynyl, butynyl, pentynyl, hexynyl and the like.

Examples of the “substituent” of the above-mentioned “C₂₋₆ alkynyl optionally having substituent(s)” include substituents selected from substituent group A. While the number of substituents is not particularly limited as long as the substituents in the designated number are substitutable, it is preferably 1 to 5, more preferably 1 to 3.

Examples of the “C₃₋₈ cycloalkyl” of the above-mentioned “C₃₋₈ cycloalkyl optionally having substituent(s)” include cyclopropyl, cyclobutyl, cyclopentyl, cyclohexyl, cycloheptyl, cyclooctyl and the like.

Examples of the “substituent” of the above-mentioned “C₃₋₈ cycloalkyl optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A. While the number of substituents is not particularly limited as long as the substituents in the designated number are substitutable, it is preferably 1 to 5, more preferably 1 to 3.

Examples of the “C₃₋₈ cycloalkenyl” of the above-mentioned “C₃₋₈ cycloalkenyl optionally having substituent(s)” include cyclopropenyl, cyclobutenyl, cyclopentenyl, cyclohexenyl, cycloheptenyl, cyclooctenyl and the like.

Examples of the “substituent” of the above-mentioned “C₃₋₈ cycloalkenyl optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A. While the number of substituents is not particularly limited as long as the substituents in the designated number are substitutable, it is preferably 1 to 5, more preferably 1 to 3.

Examples of the “C₆₋₁₀ aryl” of the above-mentioned “C₆₋₁₀ aryl optionally having substituent(s)” include phenyl, 1-naphthyl, 2-naphthyl and the like.

Examples of the “substituent” of the above-mentioned “C₆₋₁₀ aryl optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A (excluding oxo). While the number of substituents is not particularly limited as long as the substituents in the designated number are substitutable, it is preferably 1 to 5, more preferably 1 to 3.

In the present specification, the “acyl” includes
(1) formyl,
(2) alkyl-carbonyl (C₁₋₆ alkyl-carbonyl) optionally having substituent(s),
(3) alkenyl-carbonyl (C₂₋₆ alkenyl-carbonyl) optionally having substituent(s),
(4) alkynyl-carbonyl (C₂₋₆ alkynyl-carbonyl) optionally having substituent(s),
(5) cycloalkyl-carbonyl (C₃₋₈ cycloalkyl-carbonyl) optionally having substituent(s),
(6) cycloalkenyl-carbonyl (C₃₋₈ cycloalkenyl-carbonyl) optionally having substituent(s),
(7) aryl-carbonyl (C₆₋₁₀ aryl-carbonyl) optionally having substituent(s),
(8) heterocyclyl-carbonyl optionally having substituent(s),
(9) carboxyl,
(10) alkoxy-carbonyl (C₁₋₆ alkoxy-carbonyl) optionally having substituent(s),
(11) alkenyloxy-carbonyl (C₂₋₆ alkenyloxy-carbonyl) optionally having substituent(s),
(12) alkynyloxy-carbonyl (C₂₋₆ alkynyloxy-carbonyl) optionally having substituent(s),
(13) cycloalkyloxy-carbonyl (C₃₋₈ cycloalkyloxy-carbonyl) optionally having substituent(s),
(14) cycloalkenyloxy-carbonyl (C₃₋₈ cycloalkenyloxy-carbonyl) optionally having substituent(s),
(15) cycloalkynyloxy-carbonyl (C₃₋₈ cycloalkynyloxy-carbonyl) optionally having substituent(s),
(16) aryloxy-carbonyl (C₆₋₁₀ aryloxy-carbonyl) optionally having substituent(s),
(17) heterocyclyl-oxy-carbonyl optionally having substituent(s),
(18) carbamoyl optionally having substituent(s), and the like.

Examples of the “C₁₋₆ alkyl-carbonyl” of the above-mentioned “C₁₋₆ alkyl-carbonyl optionally having substituent(s)” include acetyl, ethylcarbonyl, propylcarbonyl, isopropylcarbonyl, butylcarbonyl, isobutylcarbonyl, sec-butylcarbonyl, tert-butylcarbonyl, pentylcarbonyl, hexylcarbonyl and the like.

Examples of the “substituent” of the above-mentioned “C₁₋₆ alkyl-carbonyl optionally having substituent(s)” include substituents selected from substituent group A. While the number of substituents is not particularly limited as long as the substituents in the designated number are substitutable, it is preferably 1 to 5, more preferably 1 to 3.

Examples of the “C₂₋₆ alkenyl-carbonyl” of the above-mentioned “C₂₋₆ alkenyl-carbonyl optionally having substituent(s)” include ethenylcarbonyl, propenylcarbonyl, butenylcarbonyl, pentenylcarbonyl, hexenylcarbonyl and the like.

Examples of the “substituent” of the above-mentioned “C₂₋₆ alkenyl-carbonyl optionally having substituent(s)” include substituents selected from substituent group A. While the number of substituents is not particularly limited as long as the substituents in the designated number are substitutable, it is preferably 1 to 5, more preferably 1 to 3.

Examples of the “C₂₋₆ alkynyl-carbonyl” of the above-mentioned “C₂₋₆ alkynyl-carbonyl optionally having substituent(s)” include ethynylcarbonyl, propynylcarbonyl, butynylcarbonyl, pentynylcarbonyl, hexynylcarbonyl and the like.

Examples of the “substituent” of the above-mentioned “C₂₋₆ alkynyl-carbonyl optionally having substituent(s)” include substituents selected from substituent group A. While the number of substituents is not particularly limited as long as the substituents in the designated number are substitutable, it is preferably 1 to 5, more preferably 1 to 3.

Examples of the “C₃₋₈ cycloalkyl-carbonyl” of the above-mentioned “C₃₋₈ cycloalkyl-carbonyl optionally having substituent(s)” include cyclopropylcarbonyl, cyclobutylcarbonyl, cyclopentylcarbonyl, cyclohexylcarbonyl, cycloheptylcarbonyl, cyclooctylcarbonyl and the like.

Examples of the “substituent” of the above-mentioned “C₃₋₈ cycloalkyl-carbonyl optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A. While the number of substituents is not particularly limited as long as the substituents in the designated number are substitutable, it is preferably 1 to 5, more preferably 1 to 3.

Examples of the “C₃₋₈ cycloalkenyl-carbonyl” of the above-mentioned “C₃₋₈ cycloalkenyl-carbonyl optionally having substituent(s)” include cyclopropenylcarbonyl, cyclobutenylcarbonyl, cyclopentenylcarbonyl, cyclohexenylcarbonyl, cycloheptenylcarbonyl, cyclooctenylcarbonyl and the like.

Examples of the “substituent” of the above-mentioned “C₃₋₈ cycloalkenyl-carbonyl optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A. While the number of substituents is not particularly limited as long as the substituents in the designated number are substitutable, it is preferably 1 to 5, more preferably 1 to 3.

Examples of the “C₆₋₁₀ aryl-carbonyl” of the above-mentioned “C₆₋₁₀ aryl-carbonyl optionally having substituent(s)” include benzoyl, 1-naphthoyl, 2-naphthoyl and the like.

Examples of the “substituent” of the above-mentioned “C₆₋₁₀ aryl-carbonyl optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A (excluding oxo). While the number of substituents is not particularly limited as long as the substituents in the designated number are substitutable, it is preferably 1 to 5, more preferably 1 to 3.

Examples of the “heterocycle” of the above-mentioned “heterocyclyl-carbonyl optionally having substituent(s)” include (1) 5- or 6-membered mono-cyclic aromatic heterocycle (e.g., furan, thiophene, pyrrole, oxazole, isoxazole, thiazole, isothiazole, imidazole, pyridine, pyrazole etc.), (2) 8- to 12-membered condensed aromatic heterocycle (e.g., benzofuran, isobenzofuran, benzothiophene, isobenzothiophene, indole, isoindole, 1H-indazole, benzimidazole, benzoxazole etc.), (3) 5- or 6-membered non-aromatic heterocycle (e.g., oxirane, azetidine, oxetane, pyrrolidine, tetrahydrofuran, thioran, piperidine etc.) and the like.

Examples of the “substituent” of the above-mentioned “heterocyclyl-carbonyl optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A (excluding oxo). While the number of substituents is not particularly limited as long as the substituents in the designated number are substitutable, it is preferably 1 to 5, more preferably 1 to 3.

Examples of the “C₁₋₆ alkoxy-carbonyl” of the above-mentioned “C₁₋₆ alkoxy-carbonyl optionally having substituent(s)” include methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, hexyloxycarbonyl and the like.

Examples of the “substituent” of the above-mentioned “C₁₋₆ alkoxy-carbonyl optionally having substituent(s)” include substituents selected from substituent group A. While the number of substituents is not particularly limited as long as the substituents in the designated number are substitutable, it is preferably 1 to 5, more preferably 1 to 3.

Examples of the “C₂₋₆ alkenyloxy-carbonyl” of the above-mentioned “C₂₋₆ alkenyloxy-carbonyl optionally having substituent(s)” include ethenyloxycarbonyl, propenyloxycarbonyl, butenyloxycarbonyl, pentenyloxycarbonyl, hexenyloxycarbonyl and the like.

Examples of the “substituent” of the above-mentioned “C₂₋₆ alkenyloxy-carbonyl optionally having substituent(s)” include substituents selected from substituent group A. While the number of substituents is not particularly limited as long as the substituents in the designated number are substitutable, it is preferably 1 to 5, more preferably 1 to 3.

Examples of the “C₂₋₆ alkynyloxy-carbonyl” of the above-mentioned “C₂₋₆ alkynyloxy-carbonyl optionally having substituent(s)” include ethynyloxycarbonyl, propynyloxycarbonyl, butynyloxycarbonyl, pentynyloxycarbonyl, hexynyloxycarbonyl and the like.

Examples of the “substituent” of the above-mentioned “C₂₋₆ alkynyloxy-carbonyl optionally having substituent(s)” include substituents selected from substituent group A. While the number of substituents is not particularly limited as long as the substituents in the designated number are substitutable, it is preferably 1 to 5, more preferably 1 to 3.

Examples of the “C₃₋₈ cycloalkyloxy-carbonyl” of the above-mentioned “C₃₋₈ cycloalkyloxy-carbonyl optionally having substituent(s)” include cyclopropyloxycarbonyl, cyclobutyloxycarbonyl, cyclopentyloxycarbonyl, cyclohexyloxycarbonyl, cycloheptyloxycarbonyl, cyclooctyloxycarbonyl and the like.

Examples of the “substituent” of the above-mentioned “C₃₋₈ cycloalkyloxy-carbonyl optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A. While the number of substituents is not particularly limited as long as the substituents in the designated number are substitutable, it is preferably 1 to 5, more preferably 1 to 3.

Examples of the “C₃₋₈ cycloalkenyloxy-carbonyl” of the above-mentioned “C₃₋₈ cycloalkenyloxy-carbonyl optionally having substituent(s)” include cyclopropenyloxycarbonyl, cyclobutenyloxycarbonyl, cyclopentenyloxycarbonyl, cyclohexenyloxycarbonyl, cycloheptenyloxycarbonyl, cyclooctenyloxycarbonyl and the like.

Examples of the “substituent” of the above-mentioned “C₃₋₈ cycloalkenyloxy-carbonyl optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A. While the number of substituents is not particularly limited as long as the substituents in the designated number are substitutable, it is preferably 1 to 5, more preferably 1 to 3.

Examples of the “C₃₋₈ cycloalkynyloxy-carbonyl” of the above-mentioned “C₃₋₈ cycloalkynyloxy-carbonyl optionally having substituent(s)” include cyclopropynyloxycarbonyl, cyclobutynyloxycarbonyl, cyclopentynyloxycarbonyl, cyclohexynyloxycarbonyl, cycloheptynyloxycarbonyl, cyclooctynyloxycarbonyl and the like.

Examples of the “substituent” of the above-mentioned “C₃₋₈ cycloalkynyloxy-carbonyl optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A. While the number of substituents is not particularly limited as long as the substituents in the designated number are substitutable, it is preferably 1 to 5, more preferably 1 to 3.

Examples of the “C₆₋₁₀ aryloxy-carbonyl” of the above-mentioned “C₆₋₁₀ aryloxy-carbonyl optionally having substituent(s)” include phenoxycarbonyl, 1-naphthyloxycarbonyl, 2-naphthyloxycarbonyl and the like.

Examples of the “substituent” of the above-mentioned “C₆₋₁₀ aryloxy-carbonyl optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A (excluding oxo). While the number of substituents is not particularly limited as long as the substituents in the designated number are substitutable, it is preferably 1 to 5, more preferably 1 to 3.

Examples of the “heterocycle” of the above-mentioned “heterocyclyl-oxy-carbonyl optionally having substituent(s)” include (1) 5- or 6-membered mono-cyclic aromatic heterocycle (e.g., furan, thiophene, pyrrole, oxazole, isoxazole, thiazole, isothiazole, imidazole, pyridine, pyrazole etc.), (2) 8- to 12-membered condensed aromatic heterocycle (e.g., benzofuran, isobenzofuran, benzothiophene, isobenzothiophene, indole, isoindole, 1H-indazole, benzimidazole, benzoxazole etc.), (3) 5- or 6-membered non-aromatic heterocycle (e.g., oxirane, azetidine, oxetane, pyrrolidine, tetrahydrofuran, thioran, piperidine etc.) and the like.

Examples of the “substituent” of the above-mentioned “heterocyclyl-oxy-carbonyl optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A (excluding oxo). While the number of substituents is not particularly limited as long as the substituents in the designated number are substitutable, it is preferably 1 to 5, more preferably 1 to 3.

The above-mentioned “carbamoyl optionally having substituent(s)” is carbamoyl optionally having 1 or 2 substituents selected from the aforementioned alkyl (C₁₋₆ alkyl) optionally having substituent(s), alkenyl (C₂₋₆ alkenyl) optionally having substituent(s), alkynyl (C₂₋₆ alkynyl) optionally having substituent(s), cycloalkyl (C₃₋₈ cycloalkyl) optionally having substituent(s), cycloalkenyl (C₃₋₈ cycloalkenyl) optionally having substituent(s) and aryl (C₆₋₁₀ aryl) optionally having substituent(s).

Examples of the “heterocyclic group” in the above-mentioned “heterocyclic group optionally having substituent(s) (which has a bond at carbon atom)” include an aromatic heterocyclic group (e.g., mono-cyclic aromatic heterocyclic group, condensed aromatic heterocyclic group), a nonaromatic heterocyclic group and the like.

Examples of the mono-cyclic aromatic heterocyclic group include a 5- to 7-membered mono-cyclic aromatic heterocyclic group containing, as a ring-constituting atom besides carbon atom, 1 to 4 hetero atoms selected from an oxygen atom, a sulfur atom and a nitrogen atom, and the like.

Specific examples of the mono-cyclic aromatic heterocyclic group include furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl, 6-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 2-imidazolyl, 4-imidazolyl, 5-imidazolyl), pyrazolyl (e.g., 3-pyrazolyl, 4-pyrazolyl), thiazolyl (e.g., 2-thiazolyl, 4-thiazolyl, 5-thiazolyl), isothiazolyl, oxazolyl (e.g., 2-oxazolyl, 4-oxazolyl, 5-oxazolyl), isoxazolyl, oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-4-yl), tetrazolyl (e.g., tetrazol-5-yl), triazinyl and the like.

Examples of the condensed aromatic heterocyclic group include a condensed group of a 5- to 7-membered mono-cyclic aromatic heterocyclic group containing, as a ring-constituting atom besides carbon atom, 1 to 4 hetero atoms selected from an oxygen atom, a sulfur atom and a nitrogen atom, and the like, and C₆₋₁₀ aryl and the like; condensed group of the above-mentioned 5- to 7-membered mono-cyclic aromatic heterocyclic groups, and the like.

Specific examples of the condensed aromatic heterocyclic group include quinolyl (e.g., 2-quinolyl, 3-quinolyl, 4-quinolyl, isoquinolyl), quinazolyl (e.g., 2-quinazolyl, 4-quinazolyl), quinoxalyl (e.g., 2-quinoxalyl), benzofuryl (e.g., 2-benzofuryl, 3-benzofuryl), benzothienyl (e.g., 2-benzothienyl, 3-benzothienyl), benzoxazolyl (e.g., 2-benzoxazolyl), benzothiazolyl (e.g., 2-benzothiazolyl, 5-benzothiazolyl, 6-benzothiazolyl), benzimidazolyl (e.g., benzimidazol-2-yl, benzimidazol-5-yl), indolyl (e.g., indol-3-yl, indol-4-yl, indol-5-yl, indol-6-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridyl (e.g., 1H-imidazo[4,5-b]pyridin-2-yl, 1H-imidazo[4,5-c]pyridin-2-yl), imidazopyrazinyl (e.g., 1H-imidazo[4,5-b]pyrazin-2-yl), benzisoxazolyl, benzotriazolyl, pyrazolopyridyl, pyrazolothienyl, pyrazolotriazinyl and the like.

Examples of the nonaromatic heterocyclic group include a 3- to 8-membered (preferably 5- or 6-membered) saturated or unsaturated (preferably saturated) nonaromatic heterocyclic group and the like.

As the nonaromatic heterocyclic group, mono-cyclic nonaromatic heterocyclic group or condensed nonaromatic heterocyclic group containing 1 to 4 hetero atoms selected from oxygen atom, sulfur atom and nitrogen atom, and the like can be mentioned. As the mono-cyclic nonaromatic heterocyclic group, a 3- to 8-membered (preferably 5- or 6-membered) group can be mentioned, and as the condensed nonaromatic heterocyclic group, a 8- to 12-membered group can be mentioned.

Specific examples of the nonaromatic heterocyclic group include oxiranyl, azetidinyl, oxetanyl, thietanyl, pyrrolidinyl, tetrahydrofuryl, thiolanyl, piperidinyl, tetrahydropyranyl, thianyl, morpholinyl, thiomorpholinyl, piperazinyl, azepanyl, oxepanyl, thiepanyl, oxazepanyl, thiazepanyl, azocanyl, oxocanyl, thiocanyl, oxazocanyl, thiazocanyl, dioxinyl and the like.

Examples of the “substituent” of the above-mentioned “heterocyclic group optionally having substituent(s) (which has a bond at carbon atom)” include (1) C₁₋₆ alkyl (e.g., methyl, ethyl, propyl, isopropyl, butyl, isobutyl, tert-butyl etc.) optionally having 1 to 3 substituents selected from a halogen atom and cyano, and (2) a substituent selected from substituent group A (excluding oxo). While the number of substituents is not particularly limited as long as the substituents in the designated number are substitutable, it is preferably 1 to 5, more preferably 1 to 3.

In the present specification, examples of the “group via a nitrogen atom” include (1) nitro, and (2) amino optionally having 1 or 2 of the above-mentioned “group via a carbon atom”.

In the present specification, examples of the “group via an oxygen atom” include hydroxyl optionally having one “group via a carbon atom” mentioned above.

In the present specification, examples of the “group via a sulfur atom” include mercapto optionally having one “group via a carbon atom” mentioned above or “group via a nitrogen atom” mentioned above, and the group may be oxidized.

In the present specification, as the “aromatic ring group” of the “aromatic ring group optionally having substituent(s)”, for example, aromatic hydrocarbon group, aromatic heterocyclic group (e.g., mono-cyclic aromatic heterocyclic group, condensed aromatic heterocyclic group) and the like can be mentioned.

Examples of the aromatic hydrocarbon group include C₆₋₁₀ aryl and the like. Specifically, phenyl, 1-naphthyl, 2-naphthyl and the like can be mentioned.

Examples of the mono-cyclic aromatic heterocyclic group include a 5- to 7-membered mono-cyclic aromatic heterocyclic group containing, as a ring-constituting atom besides carbon atom, 1 to 4 hetero atoms selected from an oxygen atom, a sulfur atom and a nitrogen atom, and the like.

Specific examples of the mono-cyclic aromatic heterocyclic group include furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl, 6-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl, oxazolyl (e.g., 2-oxazolyl, 4-oxazolyl, 5-oxazolyl), isoxazolyl (e.g., 3-isoxazolyl, 4-isoxazolyl, 5-isoxazolyl), oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl), triazinyl and the like.

Examples of the condensed aromatic heterocyclic group include a condensed group of a 8- to 12-membered condensed aromatic heterocyclic group containing, as a ring constituting atom besides carbon atom, 1 to 4 hetero atoms selected from an oxygen atom, a sulfur atom and a nitrogen atom.

Specific examples of the condensed aromatic heterocyclic group include quinolyl (e.g., 2-quinolyl, 3-quinolyl, 4-quinolyl, 8-quinolyl, isoquinolyl), quinazolyl (e.g., 2-quinazolyl, 4-quinazolyl), quinoxalyl (e.g., 2-quinoxalyl), benzofuryl (e.g., 2-benzofuryl, 3-benzofuryl), benzothienyl (e.g., 2-benzothienyl, 3-benzothienyl), benzoxazolyl (e.g., 2-benzoxazolyl), benzothiazolyl (e.g., 2-benzothiazolyl, 5-benzothiazolyl, 6-benzothiazolyl), benzimidazolyl (e.g., benzimidazol-1-yl, benzimidazol-2-yl, benzimidazol-5-yl), indolyl (e.g., indol-1-yl, indol-3-yl, indol-4-yl, indol-5-yl, indol-6-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridyl (e.g., 1H-imidazo[4,5-b]pyridin-2-yl, 1H-imidazo[4,5-c]pyridin-2-yl, imidazo[1,2-a]pyridin-5-yl), imidazopyrazinyl (e.g., 1H-imidazo[4,5-b]pyrazin-2-yl), imidazopyrimidinyl (e.g., imidazo[1,2-a]pyrimidin-2-yl), benzisoxazolyl, benzotriazolyl, pyrazolopyridyl, pyrazolothienyl, pyrazolotriazinyl, thiazolopyrimidinyl (e.g., 5H-[1,3]thiazolo[3,2-a]pyrimidine-7-yl), thienopyrimidinyl (e.g., 3,4-dihydrothieno[3,2-d]pyrimidin-2-yl, 3,4-dihydrothieno[2,3-d]pyrimidin-2-yl) and the like.

In the present specification, examples of the “substituent” of the “aromatic ring group optionally having substituent(s)” include (1) C₁₋₆ alkyl (e.g., methyl, ethyl, propyl, isopropyl, butyl, isobutyl, tert-butyl etc.) optionally having 1 to 3 substituents selected from a halogen atom, cyano, carbamoyl and C₃₋₈ cycloalkyl (e.g., cyclopropyl), (2) C₆₋₁₀ aryl-C₂₋₆ alkenyl (e.g., styryl) optionally having 1 to 3 halogen atoms (e.g., chloro), (3) C₂₋₆ alkynyl (e.g., ethynyl etc.), (4) C₆₋₁₀ aryl-C₁₋₆ alkyl (e.g., benzyl etc.), (5) tri-C₆₋₁₀ aryl-C₁₋₆ alkyl (e.g., trityl etc.), (6) C₂₋₆ alkenyl (e.g., ethenyl etc.) optionally having C₆₋₁₀ aryl (e.g., phenyl etc.) optionally having C₁₋₆ alkyl (e.g., methyl etc.) optionally having 1 to 3 halogen atoms (e.g., fluorine atom etc.) and (7) substituent selected from substituent group A. While the number of substituents is not particularly limited as long as the substituents in the designated number are substitutable, it is preferably 1 to 5, more preferably 1 to 3.

In the present specification, examples of the “substituent” of the “5- to 7-membered mono-cyclic aromatic heterocyclic group containing 1 to 4 hetero atoms selected from oxygen atom, sulfur atom and nitrogen atom, which optionally has substituent(s)” and “8- to 12-membered condensed aromatic heterocyclic group containing 1 to 4 hetero atoms selected from oxygen atom, sulfur atom and nitrogen atom, which optionally has substituent(s)” include those similar to the substituents recited for the “aromatic ring group optionally having substituent(s)”. While the number of substituents is not particularly limited as long as the substituents in the designated number are substitutable, it is preferably 1 to 5, more preferably 1 to 3.

In the present specification, examples of the “nonaromatic heterocyclic group” of the “nonaromatic heterocyclic group containing 1 to 4 hetero atoms selected from oxygen atom, sulfur atom and nitrogen atom, which optionally has substituent(s)” include a mono-cyclic nonaromatic heterocyclic group or condensed nonaromatic heterocyclic group containing 1 to 4 hetero atoms selected from oxygen atom, sulfur atom and nitrogen atom and the like. As the mono-cyclic nonaromatic heterocyclic group, a 3- to 8-membered (preferably 5- or 6-membered) group can be mentioned, and as the condensed nonaromatic heterocyclic group, a 8- to 12-membered group can be mentioned.

Specific examples of the mono-cyclic nonaromatic heterocyclic group include pyrrolidinyl (e.g., 1-pyrrolidinyl, 2-pyrrolidinyl), piperidinyl (e.g., piperidino, 2-piperidinyl, 3-piperidinyl, 4-piperidinyl), homopiperidinyl (e.g., homopiperidino, 2-homopiperidinyl, 3-homopiperidinyl, 4-homopiperidinyl), tetrahydropyridyl (e.g., 1,2,3,6-tetrahydropyridin-1-yl), morpholinyl (e.g., morpholino), thiomorpholinyl (e.g., thiomorpholino), piperazinyl (e.g., 1-piperazinyl, 2-piperazinyl, 3-piperazinyl), hexamethyleniminyl (e.g., hexamethylenimin-1-yl), oxazolidinyl (e.g., oxazolidin-2-yl), thiazolidinyl (e.g., thiazolidin-2-yl), imidazolidinyl (e.g., imidazolidin-2-yl, imidazolidin-3-yl), oxazolinyl (e.g., oxazolin-2-yl), thiazolinyl (e.g., thiazolin-2-yl), imidazolinyl (e.g., imidazolin-2-yl, imidazolin-3-yl), dioxolyl (e.g., 1,3-dioxol-4-yl), dioxolanyl (e.g., 1,3-dioxolan-4-yl), dihydrooxadiazolyl (e.g., 4,5-dihydro-1,2,4-oxadiazol-3-yl), 2-thioxo-1,3-oxazolidin-5-yl, pyranyl (e.g., 4-pyranyl), tetrahydropyranyl (e.g., 2-tetrahydropyranyl, 3-tetrahydropyranyl, 4-tetrahydropyranyl), thiopyranyl (e.g., 4-thiopyranyl), tetrahydrothiopyranyl (e.g., 2-tetrahydrothiopyranyl, 3-tetrahydrothiopyranyl, 4-tetrahydrothiopyranyl), 1-oxidotetrahydrothiopyranyl (e.g., 1-oxidotetrahydrothiopyran-4-yl), 1,1-dioxidotetrahydrothiopyranyl (e.g., 1,1-dioxidotetrahydrothiopyran-4-yl), tetrahydrofuryl (e.g., tetrahydrofuran-3-yl, tetrahydrofuran-2-yl), pyrazolidinyl (e.g., pyrazolidin-1-yl, pyrazolidin-3-yl), pyrazolinyl (e.g., pyrazolin-1-yl), tetrahydropyrimidinyl (e.g., tetrahydropyrimidin-1-yl), dihydrotriazolyl (e.g., 2,3-dihydro-1H-1,2,3-triazol-1-yl), tetrahydrotriazolyl (e.g., 2,3,4,5-tetrahydro-1H-1,2,3-triazol-1-yl), oxodihydrooxadiazolyl (e.g., 5-oxo-4,5-dihydro-1,2,4-oxadiazol-3-yl) and the like.

Specific examples of the condensed nonaromatic heterocyclic group include dihydroindolyl (e.g., 2,3-dihydro-1H-indol-1-yl), dihydroisoindolyl (e.g., 1,3-dihydro-2H-isoindol-2-yl), dihydrobenzofuranyl (e.g., 2,3-dihydro-1-benzofuran-5-yl), dihydrobenzodioxinyl (e.g., 2,3-dihydro-1,4-benzodioxinyl), dihydrobenzodioxepinyl (e.g., 3,4-dihydro-2H-1,5-benzodioxepinyl), tetrahydrobenzofuranyl (e.g., 4,5,6,7-tetrahydro-1-benzofuran-3-yl), chromenyl (e.g., 4H-chromen-2-yl, 2H-chromen-3-yl, 2H-chromen-7-yl), dihydroquinolinyl (e.g., 1,2-dihydroquinolin-4-yl), tetrahydroquinolinyl (e.g., 1,2,3,4-tetrahydroquinolin-4-yl), dihydroisoquinolinyl (e.g., 1,2-dihydroisoquinolin-4-yl), tetrahydroisoquinolinyl (e.g., 1,2,3,4-tetrahydroisoquinolin-4-yl, 1,2,3,4-tetrahydroisoquinolin-2-yl), dihydrophthalazinyl (e.g., 1,4-dihydrophthalazin-4-yl), tetrahydrobenzoazepinyl (e.g., 2,3,4,5-tetrahydro-1H-benzo[c]azepin-1-yl) and the like.

In the present specification, examples of the “substituent” of the “nonaromatic heterocyclic group containing 1 to 4 hetero atoms selected from oxygen atom, sulfur atom and nitrogen atom, which optionally has substituent(s)” include those similar to the substituents recited for the “aromatic ring group optionally having substituent(s)”. While the number of substituents is not particularly limited as long as the substituents in the designated number are substitutable, it is preferably 1 to 5, more preferably 1 to 3.

In the present specification, examples of the “cyanophenyl” of the “cyanophenyl group optionally having substituent(s)” include 2-cyanophenyl, 3-cyanophenyl and 4-cyanophenyl.

In the present specification, examples of the “substituent” of the “cyanophenyl group optionally having substituent(s)” include a substituent selected from (1) C₁₋₆ alkyl (e.g., methyl, ethyl, propyl, isopropyl, butyl, isobutyl, tert-butyl etc.) optionally having 1 to 3 substituents selected from a halogen atom (e.g., fluorine atom) and cyano, and (2) substituent group A (excluding oxo). While the number of substituents is not particularly limited as long as the substituents in the designated number are substitutable, it is preferably 1 to 4, more preferably 1 or 2.

In the present specification, specific examples of the “alkylidene” of the “—C(alkylidene)-” for —CR⁵′R⁶′ and “—C(alkylidene)- formed as —CR⁵R⁶—” include C₁₋₆ alkylidene such as methylidene, ethylidene, propylidene, isopropylidene, butylidene, pentylidene and the like.

In the present specification, examples of the “C₁₋₃ alkylene” of the “C₁₋₃ alkylene”, “—O(C₁₋₃ alkylene)O—” and “—O(C₁₋₃ alkylene)-” include ethylene, methylene, propylene and isopropylene.

In the present specification, examples of the “C₁₋₆ alkyl” of the “C₁₋₆ alkyl group optionally substituted by a hydroxy group” include those similar to the groups exemplified for the above-mentioned “group via a carbon atom”. While the number of substituents of the hydroxy group is not particularly limited as long as the substituents in the designated number are substitutable, it is preferably 1 to 4, more preferably 1 or 2.

In the present specification, examples of the “substituent” of the “phenyl group optionally having substituent(s)”, “pyridyl group optionally having substituent(s)”, “pyrazolyl group optionally having substituent(s)”, “imidazolyl group optionally having substituent(s)”, “furyl group optionally having substituent(s)”, “isoxazolyl group optionally having substituent(s)”, “oxazolyl group optionally having substituent(s)”, “thienyl group optionally having substituent(s)”, “thiazolyl group optionally having substituent(s)”, “pyrimidinyl group optionally having substituent(s)”, “indolyl group optionally having substituent(s)” and “quinolyl group optionally having substituent(s)” include those similar to the substituents of the above-mentioned “aromatic ring group optionally having substituent(s)”. While the number of substituents is not particularly limited as long as the substituents in the designated number are substitutable, it is preferably 1 to 4, more preferably 1 to 3.

In the present specification, specific examples of the “C₁₋₆ alkyl-carbonyloxy group” include methylcarbonyloxy, ethylcarbonyloxy, propylcarbonyloxy, isopropylcarbonyloxy, butylcarbonyloxy, isobutylcarbonyloxy, sec-butylcarbonyloxy, tert-butylcarbonyloxy, pentylcarbonyloxy, hexylcarbonyloxy and the like.

In the present specification, examples of the “C₁₋₆ alkyl group” of the “C₁₋₆ alkyl group optionally substituted by halogen atom” of the “4-cyanophenyl group optionally having substituent(s) selected from cyano group, C₁₋₆ alkyl group optionally substituted by halogen atom, and halogen atom” include those similar to the groups exemplified for the above-mentioned “group via a carbon atom”. While the number of the halogen atoms as the substituent is not particularly limited as long as the substituents in the designated number are substitutable, it is preferably 1 to 3.

While the number of substituents of the above-mentioned 4-cyanophenyl group is not particularly limited as long as the substituents in the designated number are substitutable, it is preferably 1 to 4, more preferably 1 or 2.

In the present specification, examples of the “C₁₋₆ alkyl group” of the “C₁₋₆ alkyl group optionally having substituent(s)” include those similar to the groups exemplified for the above-mentioned “group via a carbon atom”.

Examples of the “substituent” of the “C₁₋₆ alkyl group optionally having substituent(s)” for R¹ or R³ include a substituent selected from substituent group A. While the number of substituents is not particularly limited as long as the substituents in the designated number are substitutable, it is preferably 1 to 5, more preferably 1 to 3.

In the present specification, specific examples of the “C₁₋₆ alkoxy-carbonyloxy group” include methoxycarbonyloxy, ethoxycarbonyloxy, propoxycarbonyloxy, isopropoxycarbonyloxy, butoxycarbonyloxy, isobutoxycarbonyloxy, sec-butoxycarbonyloxy, tert-butoxycarbonyloxy, pentyloxycarbonyloxy, hexyloxycarbonyloxy and the like.

In the present specification, examples of the “substituent” of the “C₁₋₆ alkyl-carbonyloxy group optionally having substituent(s)” and the “C₁₋₆ alkoxy-carbonyloxy group optionally having substituent(s)” include a substituent selected from substituent group A. While the number of substituents is not particularly limited as long as the substituents in the designated number are substitutable, it is preferably 1 to 5, more preferably 1 to 3.

In the present specification, examples of the “C₁₋₆ alkyl group” of the “C₁₋₆ alkyl group optionally having 1 to 3 halogen atoms” include those similar to the groups exemplified for the above-mentioned “group via a carbon atom”.

In the present specification, examples of the “substituent” of the “phenyl group optionally having substituent(s)”, “pyridyl group optionally having substituent(s)”, “pyrimidinyl group optionally having substituent(s)”, “pyrazolyl group optionally having substituent(s)”, “imidazolyl group optionally having substituent(s)”, “thiazolyl group optionally having substituent(s)”, “oxazolyl group optionally having substituent(s)”, “oxadiazolyl group optionally having substituent(s)”, “pyrazolyl group optionally having substituent(s)”, “thienyl group optionally having substituent(s)” and “thiazolyl group optionally having substituent(s)” include those similar to the “substituent” of the above-mentioned “aromatic ring group optionally having substituent(s)”. While the number of substituents is not particularly limited as long as the substituents in the designated number are substitutable, it is preferably 1 to 4, more preferably 1 to 3.

In the present specification, examples of the “substituent” of the “oxadiazolyl group optionally having substituent(s)” and “carbamoyl group optionally having substituent(s)” of the “phenyl group optionally having substituent(s) selected from oxadiazolyl group optionally having substituent(s), carbamoyl group optionally having substituent(s), and acyl group” include a substituent selected from (1) C₁₋₆ alkyl (e.g., methyl, ethyl, propyl, isopropyl, butyl, isobutyl, tert-butyl etc.) optionally having 1 to 3 substituents selected from a halogen atom (e.g., fluorine atom) and cyano, and (2) substituent group A (excluding oxo). While the number of substituents is not particularly limited as long as the substituents in the designated number are substitutable, it is preferably 1 to 4, more preferably 1 or 2.

In the present specification, specific examples of the “C₁₋₆ alkyl-carbonylamino group” include methylcarbonylamino, ethylcarbonylamino, propylcarbonylamino, isopropylcarbonylamino, butylcarbonylamino, isobutylcarbonylamino, sec-butylcarbonylamino, tert-butylcarbonylamino, pentylcarbonylamino, hexylcarbonylamino and the like.

In the present specification, examples of the “substituent” of the “carbamoyl group optionally having substituent(s)” of the “pyridyl group optionally having substituent(s) selected from C₁₋₆ alkyl-carbonylamino group and carbamoyl group optionally having substituent(s)” include a substituent selected from (1) C₁₋₆ alkyl (e.g., methyl, ethyl, propyl, isopropyl, butyl, isobutyl, tert-butyl etc.) optionally having 1 to 3 substituents selected from a halogen atom (e.g., fluorine atom) and cyano, and (2) substituent group A (excluding oxo). While the number of substituents is not particularly limited as long as the substituents in the designated number are substitutable, it is preferably 1 to 4, more preferably 1 or 2.

In the present specification, examples of the “C₁₋₆ alkyl” include those similar to the groups exemplified for the above-mentioned “group via a carbon atom”.

In the present specification, examples of the “C₁₋₆ alkyl” of the “carbamoyl group substituted by a C₁₋₆ alkyl group optionally having a hydroxy group” include those similar to the groups exemplified for the above-mentioned “group via a carbon atom”.

Compound (I′) is now explained in detail.

In compound (I′), R¹ is (1) a hydrogen atom, (2) a group via a carbon atom, (3) a group via a nitrogen atom, (4) a group via an oxygen atom or (5) a group via a sulfur atom.

As R¹′, a group via a carbon atom is preferable, C₁₋₆ alkyl optionally having substituent(s) is more preferable, and C₁₋₆ alkyl (particularly methyl) is particularly preferable.

In compound (I′), R²′ is an aromatic ring group optionally having substituent(s).

As R²′, aromatic hydrocarbon group optionally having substituent(s), mono-cyclic aromatic heterocyclic group optionally having substituent(s) or condensed heterocycle optionally having substituent(s) is preferable. Among these, phenyl optionally having substituent(s), pyrazolyl optionally having substituent(s), imidazolyl optionally having substituent(s), furyl optionally having substituent(s), isoxazolyl optionally having substituent(s), oxazolyl optionally having substituent(s), pyridyl optionally having substituent(s), thienyl optionally having substituent(s), thiazolyl optionally having substituent(s), pyrimidinyl optionally having substituent(s), indolyl optionally having substituent(s) or quinolyl optionally having substituent(s) is preferable.

Particularly, as R²′,
(A) phenyl optionally having 1 to 3 substituents selected from
(1) a halogen atom (e.g., fluorine atom, chlorine atom, bromine atom),
(2) cyano,
(3) nitro,
(4) C₁₋₆ alkyl (e.g., methyl, tert-butyl) optionally having 1 to 3 substituents selected from (a) a halogen atom (e.g., fluorine atom), (b) cyano and (c) carbamoyl,
(5) C₃₋₆ cycloalkyl (e.g., cyclohexyl),
(6) C₁₋₆ alkoxy (e.g., methoxy) optionally having 1 to 3 halogen atoms (e.g., fluorine atom),
(7) C₆₋₁₀ aryloxy (e.g., phenoxy),
(8) C₁₋₆ alkylthio (e.g., methylthio) optionally having 1 to 3 halogen atoms (e.g., fluorine atom),
(9) C₁₋₆ alkyl-carbonyl (e.g., acetyl),
(10) C₁₋₆ alkyl-sulfonyl (e.g., methylsulfonyl),
(11) C₁₋₆ alkoxy-carbonyl (e.g., methoxycarbonyl),
(12) amino,
(13) di-C₁₋₆ alkylamino (e.g., dimethylamino),
(14) C₁₋₆ alkyl-carbonylamino (e.g., acetylamino),
(15) carbamoyl,
(16) thiocarbamoyl,
(17) carboxy,
(18) C₆₋₁₀ aryl (e.g., phenyl),
(19) C₁₋₆ alkyl-carbamoyl (e.g., methylcarbamoyl, ethylcarbamoyl, propylcarbamoyl, isopropylcarbamoyl, butylcarbamoyl, isobutylcarbamoyl, tert-butylcarbamoyl) optionally having 1 to 3 substituents selected from (a) a halogen atom (e.g., fluorine atom), (b) hydroxy, (c) C₁₋₆ alkoxy (e.g., methoxy, ethoxy) optionally having one hydroxy, (d) C₆₋₁₀ aryl (e.g., phenyl), (e) a 5- or 6-membered aromatic heterocyclic group (e.g., furyl, pyridyl), (f) carbamoyl, (g) di-C₁₋₆ alkylamino (e.g., dimethylamino), (h) C₃₋₆ cycloalkyl (e.g., cyclopentyl) optionally having one hydroxy, (i) C₁₋₆ alkylthio (e.g., methylthio), (j) C₁₋₆ alkylsulfinyl (e.g., methylsulfinyl), (k) C₁₋₆ alkylsulfonyl (e.g., methylsulfonyl) and (1) C₁₋₆ alkoxy-carbonyl (e.g., methoxycarbonyl),
(20) C₃₋₆ cycloalkyl-carbamoyl (e.g., cyclopropylcarbamoyl, cyclopentylcarbamoyl),
(21) di-C₁₋₆ alkyl-carbamoyl (e.g., dimethylcarbamoyl, diethylcarbamoyl, N-ethyl-N-methylcarbamoyl) optionally having one substituent selected from C₁₋₆ alkoxy (e.g., methoxy) and hydroxy,
(22) C₆₋₁₀ aryl-carbamoyl (e.g., phenylcarbamoyl) optionally having one C₁₋₆ alkyl (e.g., methyl),
(23) (5- or 6-membered aromatic heterocycle (e.g., thiazolyl, pyridyl) optionally having one C₁₋₆ alkyl (e.g., methyl etc.) optionally having 1 to 3 halogen atoms (e.g., fluorine atom))-carbamoyl,
(24) cyclic carbamoyl (e.g., azetidinylcarbonyl, pyrrolidinocarbonyl, morpholinocarbonyl, thiomorpholinocarbonyl dioxide, piperazinocarbonyl) optionally having 1 or 2 substituents selected from (a) hydroxy, (b) C₁₋₆ alkyl (e.g., methyl, ethyl) optionally having one hydroxy, (c) carbamoyl, (d) di-C₁₋₆ alkylamino (e.g., dimethylamino), (e) oxo and (f) C₆₋₁₀ aryl (e.g., phenyl),
(25) a 5-membered aromatic heterocyclic group (e.g., pyrazolyl, imidazolyl, triazolyl, oxadiazolyl, thiadiazolyl, isoxazolyl) optionally having one substituent selected from (a) hydroxy, (b) C₁₋₆ alkyl (e.g., methyl, propyl, isopropyl, tert-butyl) optionally having 1 to 3 halogen atoms (e.g., fluorine atom), (c) C₃₋₆ cycloalkyl (e.g., cyclopentyl), (d) cyano and (e) carbamoyl,
(26) a nonaromatic heterocyclic group (e.g., pyrrolidinyl, morpholinyl) optionally having oxo,
(27) an aromatic heterocyclic group (e.g., triazinyl)-amino-carbamoyl optionally having two C₁₋₆ alkoxy (e.g., methoxy),
(28) C₂₋₃ alkyleneoxy (e.g., ethyleneoxy) optionally having 1 or 2 substituents selected from C₁₋₆ alkyl (e.g., methyl) and oxo,
(29) C₁₋₂ alkylenedioxy (e.g., methylenedioxy),
(30) C₂₋₄ alkylene (e.g., trimethylene, tetramethylene) optionally having oxo, and,
(31) C₂₋₄ alkenyleneoxy (e.g., propenyleneoxy) optionally having 1 or 2 substituents selected from C₁₋₆ alkyl (e.g., methyl) and oxo;
(B) pyrazolyl (e.g., 1-pyrazolyl, 3-pyrazolyl, 4-pyrazolyl) optionally having 1 to 3 substituents selected from
(1) C₁₋₆ alkyl (e.g., methyl) optionally having 1 to 3 halogen atoms (e.g., fluorine atom), and
(2) C₆₋₁₀ aryl (e.g., phenyl) optionally having 1 or 2 substituents selected from a halogen atom (e.g., chlorine atom) and cyano;
(C) imidazolyl (e.g., 2-imidazolyl);
(D) furyl (e.g., 3-furyl);
(E) isoxazolyl (e.g., 3-isoxazolyl);
(F) oxazolyl (e.g., 2-oxazolyl);
(G) pyridyl (e.g., 2-pyridyl, 3-pyridyl, 4-pyridyl) optionally having 1 to 3 substituents selected from
(1) a halogen atom (e.g., fluorine atom, bromine atom),
(2) hydroxy,
(3) cyano,
(4) C₁₋₆ alkyl (e.g., methyl) optionally having 1 to 3 halogen atoms (e.g., fluorine atom),
(5) carbamoyl, and,
(6) C₁₋₆ alkyl-carbamoyl (e.g., isobutylcarbamoyl, tert-butylcarbamoyl) optionally having hydroxy;
(H) thienyl (e.g., 3-thienyl);
(I) thiazolyl (e.g., 2-thiazolyl);
(J) pyrimidinyl (e.g., 2-pyrimidinyl) optionally having one halogen atom (fluorine atom);
(K) indolyl (e.g., 4-indolyl); or
(L) quinolyl (e.g., 8-quinolyl) optionally having one halogen atom (e.g., fluorine atom) is preferable.

Among these, as R²′,
(A) phenyl optionally having 1 to 3 substituents selected from
(1) a halogen atom (e.g., fluorine atom, chlorine atom, bromine atom),
(2) cyano,
(3) nitro,
(4) C₁₋₆ alkyl (e.g., methyl, tert-butyl) optionally having 1 to 3 substituents selected from (a) a halogen atom (e.g., fluorine atom), (b) cyano and (c) carbamoyl,
(5) C₃₋₆ cycloalkyl (e.g., cyclohexyl),
(6) C₁₋₆ alkoxy (e.g., methoxy) optionally having 1 to 3 halogen atoms (e.g., fluorine atom),
(7) C₆₋₁₀ aryloxy (e.g., phenoxy),
(8) C₁₋₆ alkylthio (e.g., methylthio) optionally having 1 to 3 halogen atoms (e.g., fluorine atom),
(9) C₁₋₆ alkyl-carbonyl (e.g., acetyl),
(10) C₁₋₆ alkyl-sulfonyl (e.g., methylsulfonyl),
(11) C₁₋₆ alkoxy-carbonyl (e.g., methoxycarbonyl),
(12) amino,
(13) di-C₁₋₆ alkylamino (e.g., dimethylamino),
(14) C₁₋₆ alkyl-carbonylamino (e.g., acetylamino),
(15) carbamoyl,
(16) thiocarbamoyl,
(17) carboxy,
(18) C₆₋₁₀ aryl (e.g., phenyl),
(19) C₁₋₆ alkyl-carbamoyl (e.g., methylcarbamoyl, ethylcarbamoyl, propylcarbamoyl, isopropylcarbamoyl, butylcarbamoyl, isobutylcarbamoyl, tert-butylcarbamoyl) optionally having 1 to 3 substituents selected from (a) a halogen atom (e.g., fluorine atom), (b) hydroxy, (c) C₁₋₆ alkoxy (e.g., methoxy, ethoxy) optionally having one hydroxy, (d) C₆₋₁₀ aryl (e.g., phenyl), (e) 5- or 6-membered aromatic heterocyclic group (e.g., furyl, pyridyl), (f) carbamoyl, (g) di-C₁₋₆ alkylamino (e.g., dimethylamino), (h) C₃₋₆ cycloalkyl (e.g., cyclopentyl) optionally having one hydroxy, (i) C₁₋₆ alkylthio (e.g., methylthio), (j) C₁₋₆ alkylsulfinyl (e.g., methylsulfinyl), (k) C₁₋₆ alkylsulfonyl (e.g., methylsulfonyl) and (l) C₁₋₆ alkoxy-carbonyl (e.g., methoxycarbonyl),
(20) C₃₋₆ cycloalkyl-carbamoyl (e.g., cyclopropylcarbamoyl, cyclopentylcarbamoyl),
(21) di-C₁₋₆ alkyl-carbamoyl (e.g., dimethylcarbamoyl, diethylcarbamoyl, N-ethyl-N-methylcarbamoyl) optionally having one substituent selected from C₁₋₆ alkoxy (e.g., methoxy) and hydroxy,
(22) C₆₋₁₀ aryl-carbamoyl (e.g., phenylcarbamoyl) optionally having one C₁₋₆ alkyl (e.g., methyl),
(23) (5- or 6-membered aromatic heterocycle (e.g., thiazolyl, pyridyl) optionally having one C₁₋₆ alkyl (e.g., methyl etc.) optionally having 1 to 3 halogen atoms (e.g., fluorine atom))-carbamoyl,
(24) cyclic carbamoyl (e.g., azetidinylcarbonyl, pyrrolidinocarbonyl, morpholinocarbonyl, thiomorpholinocarbonyl dioxide, piperazinocarbonyl) optionally having 1 or 2 substituents selected from (a) hydroxy, (b) C₁₋₆ alkyl (e.g., methyl, ethyl) optionally having one hydroxy, (c) carbamoyl, (d) di-C₁₋₆ alkylamino (e.g., dimethylamino), (e) oxo and (f) C₆₋₁₀ aryl (e.g., phenyl),
(25) a 5-membered aromatic heterocyclic group (e.g., pyrazolyl, imidazolyl, triazolyl, oxadiazolyl, thiadiazolyl, isoxazolyl) optionally having one substituent selected from (a) hydroxy, (b) C₁₋₆ alkyl (e.g., methyl, propyl, isopropyl, tert-butyl) optionally having 1 to 3 halogen atoms (e.g., fluorine atom), (c) C₃₋₆ cycloalkyl (e.g., cyclopentyl), (d) cyano and (e) carbamoyl,
(26) a nonaromatic heterocyclic group (e.g., pyrrolidinyl, morpholinyl) optionally having oxo,
(27) an aromatic heterocyclic group (e.g., triazinyl)-amino-carbamoyl optionally having two C₁₋₆ alkoxy (e.g., methoxy),
(28) C₂₋₃ alkyleneoxy (e.g., ethyleneoxy) optionally having 1 or 2 substituents selected from C₁₋₆ alkyl (e.g., methyl) and oxo,
(29) C₁₋₂ alkylenedioxy (e.g., methylenedioxy),
(30) C₂₋₄ alkylene (e.g., trimethylene, tetramethylene) optionally having oxo, and,
(31) C₂₋₄ alkenyleneoxy (e.g., propenyleneoxy) optionally having 1 or 2 substituents selected from C₁₋₆ alkyl (e.g., methyl) and oxo
is preferable, and particularly, phenyl optionally having 1 to 3 substituents selected from
(1) C₁₋₆ alkyl-carbamoyl (e.g., methylcarbamoyl, ethylcarbamoyl, propylcarbamoyl, isopropylcarbamoyl, butylcarbamoyl, isobutylcarbamoyl, tert-butylcarbamoyl) optionally having 1 to 3 substituents selected from (a) a halogen atom (e.g., fluorine atom), (b) hydroxy, (c) C₁₋₆ alkoxy (e.g., methoxy, ethoxy) optionally having one hydroxy, (d) C₆₋₁₀ aryl (e.g., phenyl), (e) 5- or 6-membered aromatic heterocyclic group (e.g., furyl, pyridyl), (f) carbamoyl, (g) di-C₁₋₆ alkylamino (e.g., dimethylamino), (h) C₃₋₆ cycloalkyl (e.g., cyclopentyl) optionally having one hydroxy, (i) C₁₋₆ alkylthio (e.g., methylthio), (j) C₁₋₆ alkylsulfinyl (e.g., methylsulfinyl), (k) C₁₋₆ alkylsulfonyl (e.g., methylsulfonyl) and (1) C₁₋₆ alkoxy-carbonyl (e.g., methoxycarbonyl),
(2) cyclic carbamoyl (e.g., azetidinylcarbonyl, pyrrolidinocarbonyl, morpholinocarbonyl, thiomorpholinocarbonyl dioxide, piperazinocarbonyl) optionally having 1 or 2 substituents selected from (a) hydroxy, (b) C₁₋₆ alkyl (e.g., methyl, ethyl) optionally having one hydroxy, (c) carbamoyl, (d) di-C₁₋₆ alkylamino (e.g., dimethylamino), (e) oxo and (f) C₆₋₁₀ aryl (e.g., phenyl), and
(3) a 5-membered aromatic heterocyclic group (e.g., pyrazolyl, imidazolyl, triazolyl, oxadiazolyl, thiadiazolyl, isoxazolyl) optionally having one substituent selected from (a) hydroxy, (b) C₁₋₆ alkyl (e.g., methyl, propyl, isopropyl, tert-butyl) optionally having 1 to 3 halogen atoms (e.g., fluorine atom), (c) C₃₋₆ cycloalkyl (e.g., cyclopentyl), (d) cyano and (e) carbamoyl
is preferable.

In compound (I′), R³′ is (1) a hydrogen atom, (2) a group via a carbon atom, (3) a group via a nitrogen atom, (4) a group via an oxygen atom or (5) a group via a sulfur atom.

As R³′, a group via a carbon atom or a group via an oxygen atom is preferable, and C₁₋₆ alkyl optionally having substituent(s), C₁₋₆ alkyl-carbonyloxy optionally having substituent(s) (hydroxy substituted by C₁₋₆ alkyl-carbonyl optionally having substituent(s)) or C₁₋₆ alkoxy-carbonyloxy optionally having substituent(s) (hydroxy substituted by C₁₋₆ alkoxy-carbonyl optionally having substituent(s)) is preferable. Particularly, C₁₋₆ alkyl (particularly methyl), C₁₋₆ alkyl-carbonyloxy (particularly acetoxy) or C₁₋₆ alkoxy-carbonyloxy (particularly methoxycarbonyloxy) is preferable.

In compound (I′), R⁴′ is cyanophenyl optionally having substituent(s). As R⁴′, 4-cyanophenyl optionally having substituent(s) is preferable. Particularly, 4-cyanophenyl optionally having one substituent selected from (1) a halogen atom (e.g., fluorine atom, chlorine atom, bromine atom), (2) cyano, (3) C₁₋₆ alkyl (e.g., methyl) optionally having 1 to 3 halogen atoms (e.g., fluorine atom) and (4) C₆₋₁₀ aryl (e.g., phenyl) optionally having C₁₋₆ alkoxy-carbonyl (e.g., methoxycarbonyl) is preferable. Particularly, 4-cyanophenyl, 3,4-di-cyanophenyl, 3-bromo-4-cyanophenyl, 3-chloro-4-cyanophenyl, 4-cyano-2-fluorophenyl, 4-cyano-3-fluorophenyl, 4-cyano-3-(trifluoromethyl)phenyl, 4-cyano-2-(trifluoromethyl)phenyl or 4-cyano-3-(4-methoxycarbonylphenyl)phenyl is preferable.

In compound (I′), X′ is
(1) —Y′—CR⁵′R⁶′-Z′-
wherein R⁵′ and R⁶′ are the same or different and each is a hydrogen atom, a group via a carbon atom, a group via a nitrogen atom, a group via an oxygen atom or a group via a sulfur atom,
or —CR⁵′ R⁶— is —C(alkylidene)-,
Y′ is a bond, —COCO—, —CONH—, —COCONH— or —O—;
Z′ is a bond, —CH₂—, —CONH—, —O—, —OCH₂—, —S—, —SO—, —SO₂—, —CON(C₆H₅)— or

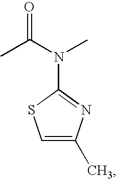

(2) —CO(CONH)ₙ— (n is 0 or 1),
(3) —NHCO—,
(4) —CONH—,
(5) —O—,
(6) —CH═CH—, or
(7) —O(C₁₋₃ alkylene)O—.

R⁵′ is preferably a hydrogen atom, a group via a carbon atom or a group via an oxygen atom, and a hydrogen atom, C₁₋₆ alkyl, hydroxy or C₆₋₁₀ aryl-carbonyloxy is more preferable. Particularly, a hydrogen atom, methyl, ethyl, hydroxy or benzoyloxy is preferable.

R⁶′ is preferably a hydrogen atom or a group via an oxygen atom, and a hydrogen atom or hydroxy is more preferable.

As the “alkylidene” of the “—C(alkylidene)-” for —CR⁵′R⁶′—, for example, C₁₋₆ alkylidene can be mentioned, with preference given to methylidene.

The “C₁₋₃ alkylene” of the “—O(C₁₋₃ alkylene)O—” for X′ is, for example, ethylene, methylene, propylene or isopropylene.

As X′, —CH₂—, —CH(Me)—, —CH(OH)—, —CH(OCOPh)-, —C(Me)(OH)—, —C(CH₂CH₃)(OH)—, —CO—, —C(═CH₂)—, —O—, —CH₂O—, —CH₂S—, —CH(Me)S—, —CH₂CH₂—, —CH₂SO₂—, (E)-CH═CH—, —NHCO—, —CONH—, —C(Me)(OH)CH₂—, —OCH₂—, —COCONH—, —C(Me)(OH)CONH—, —CONHCH₂—, —CH₂OCH₂—, —COCOCH₂SO₂— or —COCONHCH₂— is preferable, and —CH₂— or —O— is particularly preferable.

Compound (I′) does not include the following compounds.

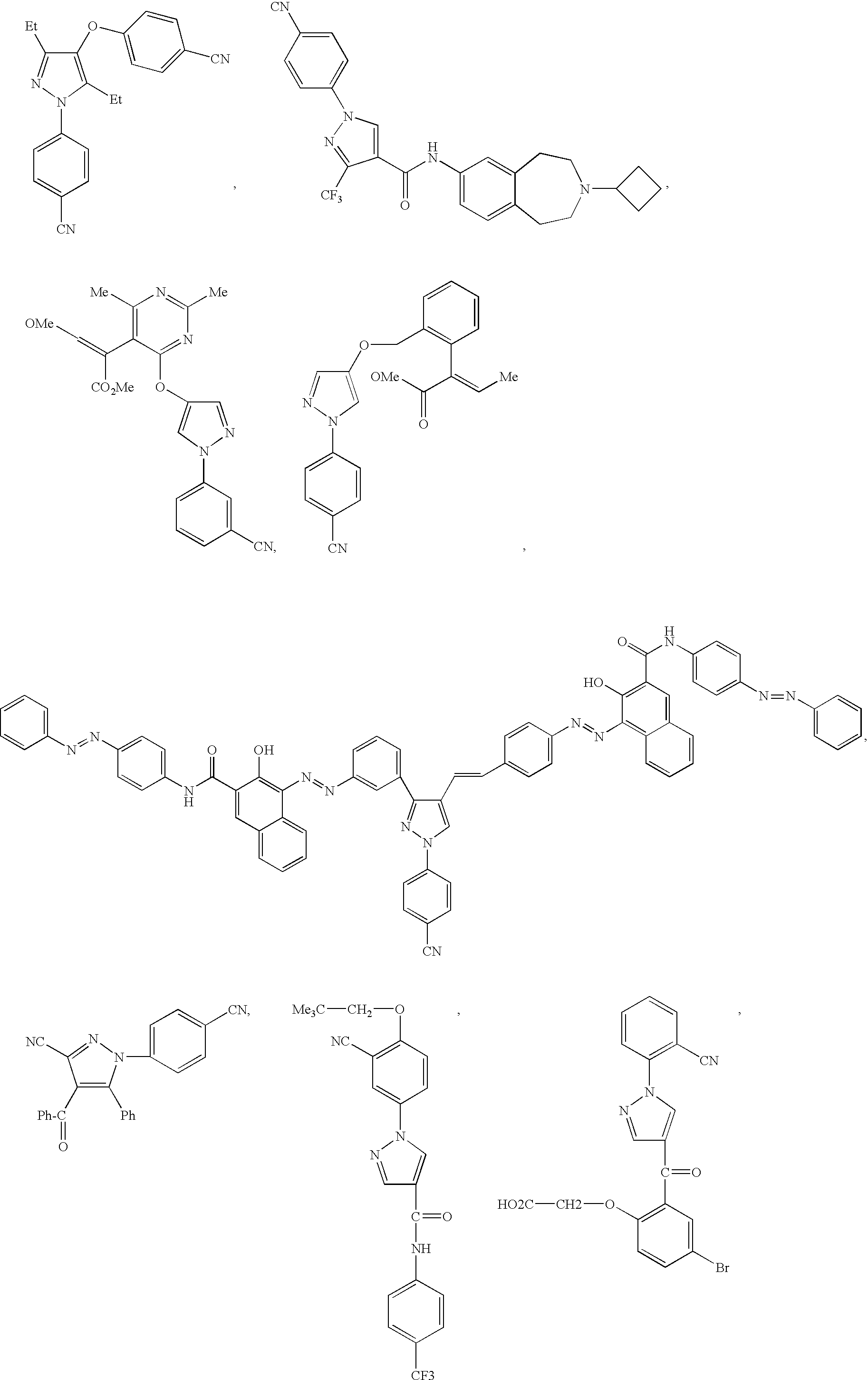

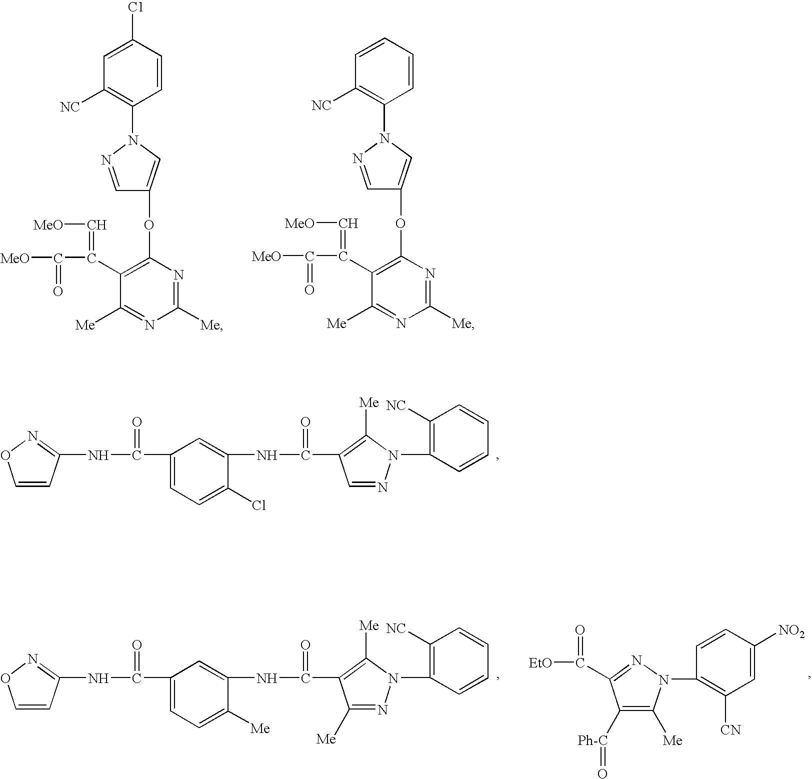

a compound represented by the formula:

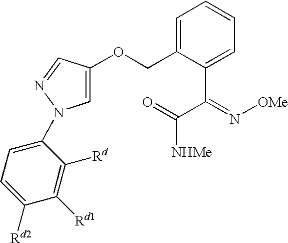

wherein one of R^{d}, R^{d1} and R^{d2} is a cyano group, and the other two groups are the same or different and each is a hydrogen atom, a chlorine atom or a methyl group, and, a compound represented by the formula:

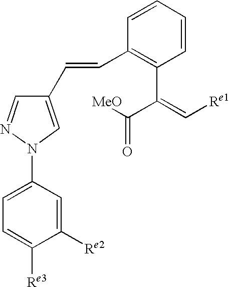

wherein R^{e1} is a methyl group, an ethyl group or a methoxymethyl group, one of R^{e2} and R^{e3} is a cyano group, and the other is a hydrogen atom.

As compound (I′), a compound wherein
R¹′ is C₁₋₆ alkyl optionally having substituent(s);
R²′ is phenyl optionally having substituent(s), pyrazolyl optionally having substituent(s), imidazolyl optionally having substituent(s), furyl optionally having substituent(s), isoxazolyl optionally having substituent(s), oxazolyl optionally having substituent(s), pyridyl optionally having substituent(s), thienyl optionally having substituent(s), thiazolyl optionally having substituent(s), pyrimidinyl optionally having substituent(s), indolyl optionally having substituent(s) or quinolyl optionally having substituent(s);
R³′ is C₁₋₆ alkyl optionally having substituent(s), C₁₋₆ alkyl-carbonyloxy optionally having substituent(s) or C₁₋₆ alkoxy-carbonyloxy optionally having substituent(s);
R⁴′ is 4-cyanophenyl optionally having substituent(s);
X′ is —CH₂—, —CH(Me)-, —CH(OH)—, —CH(OCOPh)-, —C(Me)(OH)—, —C(CH₂CH₃)(OH)—, —CO—, —C(═CH₂)—, —O—, —CH₂O—, —CH₂S—, —CH(Me)S—, —CH₂CH₂—, —CH₂SO₂—, (E)-CH═CH—, —NHCO—, —CONH—, —C(Me)(OH)CH₂—, —OCH₂—, —COCONH—, —C(Me)(OH)CONH—, —CONHCH₂—, —CH₂OCH₂—, —COCOCH₂SO₂— or —COCONHCH₂— is preferable.

Particularly, as compound (I′), a compound wherein
R¹′ is C₁₋₆ alkyl (particularly methyl);
R²′ is
(A) phenyl optionally having 1 to 3 substituents selected from
(1) a halogen atom (e.g., fluorine atom, chlorine atom, bromine atom),
(2) cyano,
(3) nitro,
(4) C₁₋₆ alkyl (e.g., methyl, tert-butyl) optionally having 1 to 3 substituents selected from (a) a halogen atom (e.g., fluorine atom), (b) cyano and (c) carbamoyl,
(5) C₃₋₆ cycloalkyl (e.g., cyclohexyl),
(6) C₁₋₆ alkoxy (e.g., methoxy) optionally having 1 to 3 halogen atoms (e.g., fluorine atom),
(7) C₆₋₁₀ aryloxy (e.g., phenoxy),
(8) C₁₋₆ alkylthio (e.g., methylthio) optionally having 1 to 3 halogen atoms (e.g., fluorine atom),
(9) C₁₋₆ alkyl-carbonyl (e.g., acetyl),
(10) C₁₋₆ alkyl-sulfonyl (e.g., methylsulfonyl),
(11) C₁₋₆ alkoxy-carbonyl (e.g., methoxycarbonyl),
(12) amino,
(13) di-C₁₋₆ alkylamino (e.g., dimethylamino),
(14) C₁₋₆ alkyl-carbonylamino (e.g., acetylamino),
(15) carbamoyl,
(16) thiocarbamoyl,
(17) carboxy,
(18) C₆₋₁₀ aryl (e.g., phenyl),
(19) C₁₋₆ alkyl-carbamoyl (e.g., methylcarbamoyl, ethylcarbamoyl, propylcarbamoyl, isopropylcarbamoyl, butylcarbamoyl, isobutylcarbamoyl, tert-butylcarbamoyl) optionally having 1 to 3 substituents selected from (a) a halogen atom (e.g., fluorine atom), (b) hydroxy, (c) C₁₋₆ alkoxy (e.g., methoxy, ethoxy) optionally having one hydroxy, (d) C₆₋₁₀ aryl (e.g., phenyl), (e) 5- or 6-membered aromatic heterocyclic group (e.g., furyl, pyridyl), (f) carbamoyl, (g) di-C₁₋₆ alkylamino (e.g., dimethylamino), (h) C₃₋₆ cycloalkyl (e.g., cyclopentyl) optionally having one hydroxy, (i) C₁₋₆ alkylthio (e.g., methylthio), (j) C₁₋₆ alkylsulfinyl (e.g., methylsulfinyl), (k) C₁₋₆ alkylsulfonyl (e.g., methylsulfonyl) and (l) C₁₋₆ alkoxy-carbonyl (e.g., methoxycarbonyl),
(20) C₃₋₆ cycloalkyl-carbamoyl (e.g., cyclopropylcarbamoyl, cyclopentylcarbamoyl),
(21) di-C₁₋₆ alkyl-carbamoyl (e.g., dimethylcarbamoyl, diethylcarbamoyl, N-ethyl-N-methylcarbamoyl) optionally having one substituent selected from C₁₋₆ alkoxy (e.g., methoxy) and hydroxy,
(22) C₆₋₁₀ aryl-carbamoyl (e.g., phenylcarbamoyl) optionally having one C₁₋₆ alkyl (e.g., methyl),
(23) (5- or 6-membered aromatic heterocycle (e.g., thiazolyl, pyridyl) optionally having one C₁₋₆ alkyl (e.g., methyl etc.) optionally having 1 to 3 halogen atoms (e.g., fluorine atom))-carbamoyl,
(24) cyclic carbamoyl (e.g., azetidinylcarbonyl, pyrrolidinocarbonyl, morpholinocarbonyl, thiomorpholinocarbonyl dioxide, piperazinocarbonyl) optionally having 1 or 2 substituents selected from (a) hydroxy, (b) C₁₋₆ alkyl (e.g., methyl, ethyl) optionally having one hydroxy, (c) carbamoyl, (d) di-C₁₋₆ alkylamino (e.g., dimethylamino), (e) oxo and (f) C₆₋₁₀ aryl (e.g., phenyl),
(25) a 5-membered aromatic heterocyclic group (e.g., pyrazolyl, imidazolyl, triazolyl, oxadiazolyl, thiadiazolyl, isoxazolyl) optionally having one substituent selected from (a) hydroxy, (b) C₁₋₆ alkyl (e.g., methyl, propyl, isopropyl, tert-butyl) optionally having 1 to 3 halogen atoms (e.g., fluorine atom), (c) C₃₋₆ cycloalkyl (e.g., cyclopentyl), (d) cyano and (e) carbamoyl,
(26) a nonaromatic heterocyclic group (e.g., pyrrolidinyl, morpholinyl) optionally having oxo,
(27) an aromatic heterocyclic group (e.g., triazinyl)-amino-carbamoyl optionally having two C₁₋₆ alkoxy (e.g., methoxy),
(28) C₂₋₃ alkyleneoxy (e.g., ethyleneoxy) optionally having 1 or 2 substituents selected from C₁₋₆ alkyl (e.g., methyl) and oxo,
(29) C₁₋₂ alkylenedioxy (e.g., methylenedioxy),
(30) C₂₋₄ alkylene (e.g., trimethylene, tetramethylene) optionally having oxo, and,
(31) C₂₋₄ alkenyleneoxy (e.g., propenyleneoxy) optionally having 1 or 2 substituents selected from C₁₋₆ alkyl (e.g., methyl) and oxo;
(B) pyrazolyl (e.g., 1-pyrazolyl,3-pyrazolyl, 4-pyrazolyl) optionally having 1 to 3 substituents selected from
(1) C₁₋₆ alkyl (e.g., methyl) optionally having 1 to 3 halogen atoms (e.g., fluorine atom), and,
(2) C₆₋₁₀ aryl (e.g., phenyl) optionally having 1 or 2 substituents selected from a halogen atom (e.g., chlorine atom) and cyano;
(C) imidazolyl (e.g., 2-imidazolyl);
(D) furyl (e.g., 3-furyl);
(E) isoxazolyl (e.g., 3-isoxazolyl);
(F) oxazolyl (e.g., 2-oxazolyl);
(G) pyridyl (e.g., 2-pyridyl, 3-pyridyl, 4-pyridyl) optionally having 1 to 3 substituents selected from
(1) a halogen atom (e.g., fluorine atom, bromine atom),
(2) hydroxy,
(3) cyano,
(4) C₁₋₆ alkyl (e.g., methyl) optionally having 1 to 3 halogen atoms (e.g., fluorine atom),
(5) carbamoyl, and
(6) C₁₋₆ alkyl-carbamoyl (e.g., isobutylcarbamoyl, tert-butylcarbamoyl) optionally having hydroxy;
(H) thienyl (e.g., 3-thienyl);
(I) thiazolyl (e.g., 2-thiazolyl);
(J) pyrimidinyl (e.g., 2-pyrimidinyl) optionally having one halogen atom (e.g., fluorine atom);
(K) indolyl (e.g., 4-indolyl); or
(L) quinolyl (e.g., 8-quinolyl) optionally having one halogen atom (e.g., fluorine atom)
R³′ is C₁₋₆ alkyl (particularly methyl), C₁₋₆ alkyl-carbonyloxy (particularly acetoxy) or C₁₋₆ alkoxy-carbonyloxy (particularly methoxycarbonyloxy);
R⁴′ is 4-cyanophenyl optionally having one substituent selected from (1) a halogen atom (e.g., fluorine atom, chlorine atom, bromine atom), (2) cyano, (3) C₁₋₆ alkyl (e.g., methyl) optionally having 1 to 3 halogen atoms (e.g., fluorine atom) and (4) C₆₋₁₀ aryl (e.g., phenyl) optionally having C₁₋₆ alkoxy-carbonyl (e.g., methoxycarbonyl); and
X′ is —CH₂—, —CH(Me)-, —CH(OH)—, —CH(OCOPh)-, —C(Me)(OH)—, —C(CH₂CH₃)(OH)—, —CO—, —C(═CH₂)—, —O—, —CH₂O—, —CH₂S—, —CH(Me)S—, —CH₂CH₂—, —CH₂SO₂—, (E)-CH═CH—, —NHCO—, —CONH—, —C(Me)(OH)CH₂—, —OCH₂—, —COCONH—, —C(Me)(OH)CONH—, —CONHCH₂—, —CH₂OCH₂—, —COCOCH₂SO₂— or —COCONHCH₂—;
is preferable.

Particularly, as compound (I′), a compound wherein
R¹′ is methyl;
R²′ is phenyl optionally having 1 to 3 substituents selected from
(1) C₁₋₆ alkyl-carbamoyl (e.g., methylcarbamoyl, ethylcarbamoyl, propylcarbamoyl, isopropylcarbamoyl, butylcarbamoyl, isobutylcarbamoyl, tert-butylcarbamoyl) optionally having 1 to 3 substituents selected from (a) a halogen atom (e.g., fluorine atom), (b) hydroxy, (c) C₁₋₆ alkoxy (e.g., methoxy, ethoxy) optionally having one hydroxy, (d) C₆₋₁₀ aryl (e.g., phenyl), (e) 5- or 6-membered aromatic heterocyclic group (e.g., furyl, pyridyl), (f) carbamoyl, (g) di-C₁₋₆ alkylamino (e.g., dimethylamino), (h) C₃₋₆ cycloalkyl (e.g., cyclopentyl) optionally having one hydroxy, (i) C₁₋₆ alkylthio (e.g., methylthio), (j) C₁₋₆ alkylsulfinyl (e.g., methylsulfinyl), (k) C₁₋₆ alkylsulfonyl (e.g., methylsulfonyl) and (1) C₁₋₆ alkoxy-carbonyl (e.g., methoxycarbonyl),
(2) cyclic carbamoyl (e.g., azetidinylcarbonyl, pyrrolidinocarbonyl, morpholinocarbonyl, thiomorpholinocarbonyl dioxide, piperazinocarbonyl) optionally having 1 or 2 substituents selected from (a) hydroxy, (b) C₁₋₆ alkyl (e.g., methyl, ethyl) optionally having one hydroxy, (c)carbamoyl, (d) di-C₁₋₆ alkylamino (e.g., dimethylamino), (e) oxo and (f) C₆₋₁₀ aryl (e.g., phenyl), and
(3) a 5-membered aromatic heterocyclic group (e.g., pyrazolyl, imidazolyl, triazolyl, oxadiazolyl, thiadiazolyl, isoxazolyl) optionally having one substituent selected from(a) hydroxy, (b) optionally having 1 to 3 halogen atoms (e.g., fluorine atom) C₁₋₆ alkyl (e.g., methyl, propyl, isopropyl, tert-butyl), (c) C₃₋₆ cycloalkyl (e.g., cyclopentyl), (d) cyano and (e) carbamoyl;
R³′ is methyl, acetoxy or methoxycarbonyloxy;
R⁴′ is 4-cyanophenyl, 3,4-di-cyanophenyl, 3-bromo-4-cyanophenyl, 3-chloro-4-cyanophenyl, 4-cyano-2-fluorophenyl, 4-cyano-3-fluorophenyl, 4-cyano-3-(trifluoromethyl)phenyl, 4-cyano-2-(trifluoromethyl)phenyl or 4-cyano-3-(4-methoxycarbonylphenyl)phenyl; and
X′ is —CH₂— or —O—;
is preferable.

More particularly, as compound (I′), a compound wherein
R¹′ is methyl;
R²′ is phenyl optionally having 1 to 3 substituents selected from
(1) C₁₋₆ alkyl-carbamoyl (e.g., methylcarbamoyl, ethylcarbamoyl, propylcarbamoyl, isopropylcarbamoyl, butylcarbamoyl, isobutylcarbamoyl, tert-butylcarbamoyl) optionally having 1 to 3 substituents selected from (a) a halogen atom (e.g., fluorine atom), (b) hydroxy, (c) C₁₋₆ alkoxy (e.g., methoxy, ethoxy) optionally having one hydroxy, (d) C₆₋₁₀ aryl (e.g., phenyl), (e) 5- or 6-membered aromatic heterocyclic group (e.g., furyl, pyridyl), (f) carbamoyl, (g) di-C₁₋₆ alkylamino (e.g., dimethylamino), (h) C₃₋₆ cycloalkyl (e.g., cyclopentyl) optionally having one hydroxy, (i) C₁₋₆ alkylthio (e.g., methylthio), (j) C₁₋₆ alkylsulfinyl (e.g., methylsulfinyl), (k) C₁₋₆ alkylsulfonyl (e.g., methylsulfonyl) and (l) C₁₋₆ alkoxy-carbonyl (e.g., methoxycarbonyl),
(2) cyclic carbamoyl (e.g., azetidinylcarbonyl, pyrrolidinocarbonyl, morpholinocarbonyl, thiomorpholinocarbonyl dioxide, piperazinocarbonyl) optionally having 1 or 2 substituents selected from (a) hydroxy, (b) C₁₋₆ alkyl (e.g., methyl, ethyl) optionally having one hydroxy, (c) carbamoyl, (d) di-C₁₋₆ alkylamino (e.g., dimethylamino), (e) oxo and (f) C₆₋₁₀ aryl (e.g., phenyl), and
(3) a 5-membered aromatic heterocyclic group (e.g., pyrazolyl, imidazolyl, triazolyl, oxadiazolyl, thiadiazolyl, isoxazolyl) optionally having one substituent selected from (a) hydroxy, (b) C₁₋₆ alkyl (e.g., methyl, propyl, isopropyl, tert-butyl) optionally having 1 to 3 halogen atoms (e.g., fluorine atom), (c) C₃₋₆cycloalkyl (e.g., cyclopentyl), (d) cyano and (e) carbamoyl;
R³′ is methyl, acetoxy or methoxycarbonyloxy; R⁴′ is 4-cyanophenyl, 3,4-di-cyanophenyl, 3-bromo-4-cyanophenyl, 3-chloro-4-cyanophenyl, 4-cyano-2-fluorophenyl, 4-cyano-3-fluorophenyl, 4-cyano-3-(trifluoromethyl)phenyl, 4-cyano-2-(trifluoromethyl)phenyl or 4-cyano-3-(4-methoxycarbonylphenyl)phenyl; and
X′ is —CH₂— or —O— is preferable.

More specifically, as compound (I′), the following compounds or a salt thereof is preferable.
(1) 5-(4-{[1-(3-chloro-4-cyanophenyl)-3,5-dimethyl-1H-pyrazol-4-yl]methyl}phenyl)-1,3,4-oxadiazole-2-carboxamide;
(2) N-tert-butyl-4-{[1-(3-chloro-4-cyanophenyl)-3,5-dimethyl-1H-pyrazol-4-yl]methyl}benzamide;
(3) 2-chloro-4-(4-{4-[(4-hydroxypiperidin-1-yl)carbonyl]benzyl}-3,5-dimethyl-1H-pyrazol-1-yl)benzonitrile;
(4) 4-{[1-(3-chloro-4-cyanophenyl)-3,5-dimethyl-1H-pyrazol-4-yl]methyl}-N-(2-hydroxy-2-methylpropyl)benzamide;
(5) 2-chloro-4-{3,5-dimethyl-4-[4-(morpholin-4-ylcarbonyl)phenoxy]-1H-pyrazol-1-yl}benzonitrile;
(6) 2-chloro-4-(4-{4-[(3-hydroxyazetidin-1-yl)carbonyl]benzyl}-3,5-dimethyl-1H-pyrazol-1-yl)benzonitrile;
(7) 2-chloro-4-(4-{4-[(1,1-dioxidothiomorpholin-4-yl)carbonyl]phenoxy}-3,5-dimethyl-1H-pyrazol-1-yl)benzonitrile; and
(8) N-(6-{[1-(3-chloro-4-cyanophenyl)-3,5-dimethyl-1H-pyrazol-4-yl]oxy}pyridin-3-yl)-2,2-dimethylpropanamide.

Compound (I) is now explained in detail in the following.

In compound (I), R¹ is (1) hydrogen atom, (2) a group via a carbon atom, (3) a group via a nitrogen atom, (4) a group via an oxygen atom or (5) a group via a sulfur atom.

As R¹, a group via a carbon atom is preferable, and C₁₋₆ alkyl optionally having substituent(s) is particularly preferable. Of these, C₁₋₆ alkyl (particularly methyl) is preferable.

In compound (I), R² is an aromatic ring group optionally having substituent(s).

As R², an aromatic hydrocarbon group optionally having substituent(s), mono-cyclic aromatic heterocyclic group optionally having substituent(s) or condensed heterocyclic group optionally having substituent(s) is preferable. Among these, phenyl optionally having substituent(s), pyrazolyl optionally having substituent(s), imidazolyl optionally having substituent(s), furyl optionally having substituent(s), isoxazolyl optionally having substituent(s), oxazolyl optionally having substituent(s), pyridyl optionally having substituent(s), thienyl optionally having substituent(s), thiazolyl optionally having substituent(s), pyrimidinyl optionally having substituent(s), indolyl optionally having substituent(s) or quinolyl optionally having substituent(s) is preferable.

Particularly,
(A) phenyl optionally having 1 to 3 substituents selected from
(1) a halogen atom (e.g., fluorine atom, chlorine atom, bromine atom),
(2) cyano,
(3) nitro,
(4) C₁₋₆ alkyl (e.g., methyl, tert-butyl) optionally having 1 to 3 substituents selected from (a) a halogen atom (e.g., fluorine atom), (b) cyano and (c) carbamoyl,
(5) C₃₋₆ cycloalkyl (e.g., cyclohexyl),
(6) C₁₋₆ alkoxy (e.g., methoxy) optionally having 1 to 3 halogen atoms (e.g., fluorine atom),
(7) C₆₋₁₀ aryloxy (e.g., phenoxy),
(8) C₁₋₆ alkylthio (e.g., methylthio) optionally having 1 to 3 halogen atoms (e.g., fluorine atom),
(9) C₁₋₆ alkyl-carbonyl (e.g., acetyl),
(10) C₁₋₆ alkyl-sulfonyl (e.g., methylsulfonyl),
(11) C₁₋₆ alkoxy-carbonyl (e.g., methoxycarbonyl),
(12) amino,
(13) di-C₁₋₆ alkylamino (e.g., dimethylamino),
(14) C₁₋₆ alkyl-carbonylamino (e.g., acetylamino),
(15) carbamoyl,
(16) thiocarbamoyl,
(17) carboxy,
(18) C₆₋₁₀ aryl (e.g., phenyl),
(19) C₁₋₆ alkyl-carbamoyl (e.g., methylcarbamoyl, ethylcarbamoyl, propylcarbamoyl, isopropylcarbamoyl, butylcarbamoyl, isobutylcarbamoyl, tert-butylcarbamoyl) optionally having 1 to 3 substituents selected from (a) a halogen atom (e.g., fluorine atom), (b) hydroxy, (c) C₁₋₆ alkoxy (e.g., methoxy, ethoxy) optionally having one hydroxy, (d) C₆₋₁₀ aryl (e.g., phenyl), (e) 5- or 6-membered aromatic heterocyclic group (e.g., furyl, pyridyl), (f) carbamoyl, (g) di-C₁₋₆ alkylamino (e.g., dimethylamino), (h) C₃₋₆ cycloalkyl (e.g., cyclopentyl) optionally having one hydroxy, (i) C₁₋₆ alkylthio (e.g., methylthio), (j) C₁₋₆ alkylsulfinyl (e.g., methylsulfinyl), (k) C₁₋₆ alkylsulfonyl (e.g., methylsulfonyl) and (l) C₁₋₆ alkoxy-carbonyl (e.g., methoxycarbonyl),
(20) C₃₋₆ cycloalkyl-carbamoyl (e.g., cyclopropylcarbamoyl, cyclopentylcarbamoyl),
(21) di-C₁₋₆ alkyl-carbamoyl (e.g., dimethylcarbamoyl, diethylcarbamoyl, N-ethyl-N-methylcarbamoyl) optionally having one substituent selected from C₁₋₆ alkoxy (e.g., methoxy) and hydroxy,
(22) C₆₋₁₀ aryl-carbamoyl (e.g., phenylcarbamoyl) optionally having one C₁₋₆ alkyl (e.g., methyl),
(23) (5- or 6-membered aromatic heterocycle (e.g., thiazolyl, pyridyl) optionally having one C₁₋₆ alkyl (e.g., methyl etc.) optionally having 1 to 3 halogen atoms (e.g., fluorine atom))-carbamoyl,
(24) cyclic carbamoyl (e.g., azetidinylcarbonyl, pyrrolidinocarbonyl, morpholinocarbonyl, thiomorpholinocarbonyl dioxide, piperazinocarbonyl) optionally having 1 or 2 substituents selected from (a) hydroxy, (b) C₁₋₆ alkyl (e.g., methyl, ethyl) optionally having one hydroxy, (c) carbamoyl, (d) di-C₁₋₆ alkylamino (e.g., dimethylamino), (e) oxo and (f) C₆₋₁₀ aryl (e.g., phenyl),
(25) a 5-membered aromatic heterocyclic group (e.g., pyrazolyl, imidazolyl, triazolyl, oxadiazolyl, thiadiazolyl, isoxazolyl) optionally having one substituent selected from (a) hydroxy, (b) C₁₋₆ alkyl (e.g., methyl, propyl, isopropyl, tert-butyl) optionally having 1 to 3 halogen atoms (e.g., fluorine atom), (c) C₃₋₆ cycloalkyl (e.g., cyclopentyl), (d) cyano and (e) carbamoyl,
(26) a nonaromatic heterocyclic group (e.g., pyrrolidinyl, morpholinyl) optionally having oxo,
(27) an aromatic heterocyclic group (e.g., triazinyl)-amino-carbamoyl optionally having two C₁₋₆ alkoxy (e.g., methoxy),
(28) C₂₋₃ alkyleneoxy (e.g., ethyleneoxy) optionally having 1 or 2 substituents selected from C₁₋₆ alkyl (e.g., methyl) and oxo,
(29) C₁₋₂ alkylenedioxy (e.g., methylenedioxy),
(30) C₂₋₄ alkylene (e.g., trimethylene, tetramethylene) optionally having oxo, and,
(31) C₂₋₄ alkenyleneoxy (e.g., propenyleneoxy) optionally having 1 or 2 substituents selected from C₁₋₆ alkyl (e.g., methyl) and oxo;
(B) pyrazolyl (e.g., 1-pyrazolyl, 3-pyrazolyl, 4-pyrazolyl) optionally having 1 to 3 substituents selected from
(1) C₁₋₆ alkyl (e.g., methyl) optionally having 1 to 3 halogen atoms (e.g., fluorine atom), and
(2) C₆₋₁₀ aryl (e.g., phenyl) optionally having 1 or 2 substituents selected from a halogen atom (e.g., chlorine atom) and cyano;
(C) imidazolyl (e.g., 2-imidazolyl);
(D) furyl (e.g., 3-furyl);
(E) isoxazolyl (e.g., 3-isoxazolyl);
(F) oxazolyl (e.g., 2-oxazolyl);
(G) pyridyl (e.g., 2-pyridyl, 3-pyridyl, 4-pyridyl) optionally having 1 to 3 substituents selected from
(1) a halogen atom (e.g., fluorine atom, bromine atom),
(2) hydroxy,
(3) cyano,
(4) C₁₋₆ alkyl (e.g., methyl) optionally having 1 to 3 halogen atoms (e.g., fluorine atom),
(5) carbamoyl, and,
(6) C₁₋₆ alkyl-carbamoyl (e.g., isobutylcarbamoyl, tert-butylcarbamoyl) optionally having hydroxy;
(H) thienyl (e.g., 3-thienyl);
(I) thiazolyl (e.g., 2-thiazolyl);
(J) pyrimidinyl (e.g., 2-pyrimidinyl) optionally having one halogen atom (e.g., fluorine atom);
(K) indolyl (e.g., 4-indolyl); or
(L) quinolyl (e.g., 8-quinolyl) optionally having one halogen atom (e.g., fluorine atom)
is preferable.

Particularly, as R², phenyl optionally having 1 to 3 substituents selected from
(1) a halogen atom (e.g., fluorine atom, chlorine atom, bromine atom),
(2) cyano,
(3) nitro,
(4) C₁₋₆ alkyl (e.g., methyl, tert-butyl) optionally having 1 to 3 substituents selected from (a) a halogen atom (e.g., fluorine atom), (b) cyano and (c) carbamoyl,
(5) C₃₋₆ cycloalkyl (e.g., cyclohexyl),
(6) C₁₋₆ alkoxy (e.g., methoxy) optionally having 1 to 3 halogen atoms (e.g., fluorine atom),
(7) C₆₋₁₀ aryloxy (e.g., phenoxy),
(8) C₁₋₆ alkylthio (e.g., methylthio) optionally having 1 to 3 halogen atoms (e.g., fluorine atom),
(9) C₁₋₆ alkyl-carbonyl (e.g., acetyl),
(10) C₁₋₆ alkyl-sulfonyl (e.g., methylsulfonyl),
(11) C₁₋₆ alkoxy-carbonyl (e.g., methoxycarbonyl),
(12) amino,
(13) di-C₁₋₆ alkylamino (e.g., dimethylamino),
(14) C₁₋₆ alkyl-carbonylamino (e.g., acetylamino),
(15) carbamoyl,
(16) thiocarbamoyl,
(17) carboxy,
(18) C₆₋₁₀ aryl (e.g., phenyl),
(19) C₁₋₆ alkyl-carbamoyl (e.g., methylcarbamoyl, ethylcarbamoyl, propylcarbamoyl, isopropylcarbamoyl, butylcarbamoyl, isobutylcarbamoyl, tert-butylcarbamoyl) optionally having 1 to 3 substituents selected from (a) a halogen atom (e.g., fluorine atom), (b) hydroxy, (c) C₁₋₆ alkoxy (e.g., methoxy, ethoxy) optionally having one hydroxy, (d) C₆₋₁₀ aryl (e.g., phenyl), (e) 5- or 6-membered aromatic heterocyclic group (e.g., furyl, pyridyl), (f) carbamoyl, (g) di-C₁₋₆ alkylamino (e.g., dimethylamino), (h) C₃₋₆ cycloalkyl (e.g., cyclopentyl) optionally having one hydroxy, (i) C₁₋₆ alkylthio (e.g., methylthio), (j) C₁₋₆ alkylsulfinyl (e.g., methylsulfinyl), (k) C₁₋₆ alkylsulfonyl (e.g., methylsulfonyl) and (l) C₁₋₆ alkoxy-carbonyl (e.g., methoxycarbonyl),
(20) C₃₋₆ cycloalkyl-carbamoyl (e.g., cyclopropylcarbamoyl, cyclopentylcarbamoyl),
(21) di-C₁₋₆ alkyl-carbamoyl (e.g., dimethylcarbamoyl, diethylcarbamoyl, N-ethyl-N-methylcarbamoyl) optionally having one substituent selected from C₁₋₆ alkoxy (e.g., methoxy) and hydroxy,
(22) C₆₋₁₀ aryl-carbamoyl (e.g., phenylcarbamoyl) optionally having one C₁₋₆ alkyl (e.g., methyl),
(23) (5- or 6-membered aromatic heterocycle (e.g., thiazolyl, pyridyl) optionally having one C₁₋₆ alkyl (e.g., methyl etc.) optionally having 1 to 3 halogen atoms (e.g., fluorine atom))-carbamoyl,
(24) cyclic carbamoyl (e.g., azetidinylcarbonyl, pyrrolidinocarbonyl, morpholinocarbonyl, thiomorpholinocarbonyl dioxide, piperazinocarbonyl) optionally having 1 or 2 substituents selected from (a) hydroxy, (b) C₁₋₆ alkyl (e.g., methyl, ethyl) optionally having one hydroxy, (c) carbamoyl, (d) di-C₁₋₆ alkylamino (e.g., dimethylamino), (e) oxo and (f) C₆₋₁₀ aryl (e.g., phenyl),
(25) a 5-membered aromatic heterocyclic group (e.g., pyrazolyl, imidazolyl, triazolyl, oxadiazolyl, thiadiazolyl, isoxazolyl) optionally having one substituent selected from (a) hydroxy, (b) C₁₋₆ alkyl (e.g., methyl, propyl, isopropyl, tert-butyl) optionally having 1 to 3 halogen atoms (e.g., fluorine atom), (c) C₃₋₆ cycloalkyl (e.g., cyclopentyl), (d) cyano and (e) carbamoyl,
(26) a nonaromatic heterocyclic group (e.g., pyrrolidinyl, morpholinyl) optionally having oxo,
(27) an aromatic heterocyclic group (e.g., triazinyl)-amino-carbamoyl optionally having two C₁₋₆ alkoxy (e.g., methoxy),
(28) C₂₋₃ alkyleneoxy (e.g., ethyleneoxy) optionally having 1 or 2 substituents selected from C₁₋₆ alkyl (e.g., methyl) and oxo,
(29) C₁₋₂ alkylenedioxy (e.g., methylenedioxy),
(30) C₂₋₄ alkylene (e.g., trimethylene, tetramethylene) optionally having oxo, and,
(31) C₂₋₄ alkenyleneoxy (e.g., propenyleneoxy) optionally having 1 or 2 substituents selected from C₁₋₆ alkyl (e.g., methyl) and oxo
is preferable, and particularly, phenyl optionally having 1 to 3 substituents selected from
(1) C₁₋₆ alkyl-carbamoyl (e.g., methylcarbamoyl, ethylcarbamoyl, propylcarbamoyl, isopropylcarbamoyl, butylcarbamoyl, isobutylcarbamoyl, tert-butylcarbamoyl) optionally having 1 to 3 substituents selected from (a) a halogen atom (e.g., fluorine atom), (b) hydroxy, (c) C₁₋₆ alkoxy (e.g., methoxy, ethoxy) optionally having one hydroxy, (d) C₆₋₁₀ aryl (e.g., phenyl), (e) 5- or 6-membered aromatic heterocyclic group (e.g., furyl, pyridyl), (f) carbamoyl, (g) di-C₁₋₆ alkylamino (e.g., dimethylamino), (h) C₃₋₆ cycloalkyl (e.g., cyclopentyl) optionally having one hydroxy, (i) C₁₋₆ alkylthio (e.g., methylthio), (j) C₁₋₆ alkylsulfinyl (e.g., methylsulfinyl), (k) C₁₋₆ alkylsulfonyl (e.g., methylsulfonyl) and (1) C₁₋₆ alkoxy-carbonyl (e.g., methoxycarbonyl),
(2) cyclic carbamoyl (e.g., azetidinylcarbonyl, pyrrolidinocarbonyl, morpholinocarbonyl, thiomorpholinocarbonyl dioxide, piperazinocarbonyl) optionally having 1 or 2 substituents selected from (a) hydroxy, (b) C₁₋₆ alkyl (e.g., methyl, ethyl) optionally having one hydroxy, (c) carbamoyl, (d) di-C₁₋₆ alkylamino (e.g., dimethylamino), (e) oxo and (f) C₆₋₁₀ aryl (e.g., phenyl), and
(3) a 5-membered aromatic heterocyclic group (e.g., pyrazolyl, imidazolyl, triazolyl, oxadiazolyl, thiadiazolyl, isoxazolyl) optionally having one substituent selected from (a) hydroxy, (b) C₁₋₆ alkyl (e.g., methyl, propyl, isopropyl, tert-butyl) optionally having 1 to 3 halogen atoms (e.g., fluorine atom), (c) C₃₋₆ cycloalkyl (e.g., cyclopentyl), (d) cyano and (e) carbamoyl
is preferable.

In compound (I), R³ is (1) a hydrogen atom, (2) a group via a carbon atom, (3) a group via a nitrogen atom, (4) a group via an oxygen atom or (5) a group via a sulfur atom.

As R³, a group via a carbon atom or a group via an oxygen atom is preferable, and C₁₋₆ alkyl optionally having substituent(s), C₁₋₆ alkyl-carbonyloxy optionally having substituent(s) (hydroxy substituted by C₁₋₆ alkyl-carbonyl optionally having substituent(s)) or C₁₋₆ alkoxy-carbonyloxy optionally having substituent(s) (hydroxy substituted by C₁₋₆ alkoxy-carbonyl optionally having substituent(s)) is more preferable. Particularly, C₁₋₆ alkyl (particularly methyl), C₁₋₆ alkyl-carbonyloxy (particularly acetoxy) or C₁₋₆ alkoxy-carbonyloxy (particularly methoxycarbonyloxy) is preferable.

In compound (I), R⁴ is cyanophenyl optionally having substituent(s). As R⁴, 4-cyanophenyl optionally having substituent(s) is preferable. Particularly, 4-cyanophenyl optionally having one substituent selected from (1) a halogen atom (e.g., fluorine atom, chlorine atom, bromine atom), (2) cyano, (3) C₁₋₆ alkyl (e.g., methyl) optionally having 1 to 3 halogen atoms (e.g., fluorine atom) and (4) C₆₋₁₀ aryl (e.g., phenyl) optionally having C₁₋₆ alkoxy-carbonyl (e.g., methoxycarbonyl) is preferable. Particularly, 4-cyanophenyl, 3,4-di-cyanophenyl, 3-bromo-4-cyanophenyl, 3-chloro-4-cyanophenyl, 4-cyano-2-fluorophenyl, 4-cyano-3-fluorophenyl, 4-cyano-3-(trifluoromethyl)phenyl, 4-cyano-2-(trifluoromethyl)phenyl or 4-cyano-3-(4-methoxycarbonylphenyl)phenyl is preferable.

In compound (I), X is
(1) —Y—CR⁵R⁶-Z-
wherein R⁵ and R⁶ are the same or different and each is a hydrogen atom, a group via a carbon atom, a group via a nitrogen atom, a group via an oxygen atom or a group via a sulfur atom,
or —CR⁵R⁶— is —C(alkylidene)-,
Y is a bond, —COCO—, —CONH—, —COCONH— or —O—;
Z is a bond, —CH₂—, —CONH—, —O—, —OCH₂—, —S—, —SO— or —SO₂—,
(2) —CO(CONH)ₙ— (n is 0 or 1),
(3) —NHCO—,
(4) —CONH—,
(5) —O— or
(6) —CH═CH—.

R⁵ is preferably a hydrogen atom, a group via a carbon atom or a group via an oxygen atom, and a hydrogen atom, C₁₋₆ alkyl, hydroxy or C₆₋₁₀ aryl-carbonyloxy is preferable. Particularly, a hydrogen atom, methyl, ethyl, hydroxy or benzoyloxy is preferable.

R⁶ is preferably a hydrogen atom or a group via an oxygen atom, and a hydrogen atom or hydroxy is preferable.

As the “alkylidene” of “—C(alkylidene)- for —CR⁵R⁶—”, for example, C₁₋₆ alkylidene can be mentioned, and methylidene is preferable.

As X, —CH₂—, —CH(Me)-, —CH(OH)—, —CH(OCOPh)-, —C(Me)(OH)—, —C(CH₂CH₃)(OH)—, —CO—, —C(═CH₂)—, —O—, —CH₂O—, —CH₂S—, —CH(Me)S—, —CH₂CH₂—, —CH₂SO₂—, (E)-CH═CH—, —NHCO—, —CONH—, —C(Me)(OH)CH₂—, —OCH₂—, —COCONH—, —C(Me)(OH)CONH—, —CONHCH₂—, —CH₂OCH₂—, —COCOCH₂SO₂— or —COCONHCH₂— is preferable, and —CH₂— or —O— is particularly preferable.

Compound (I) does not include the following compounds.

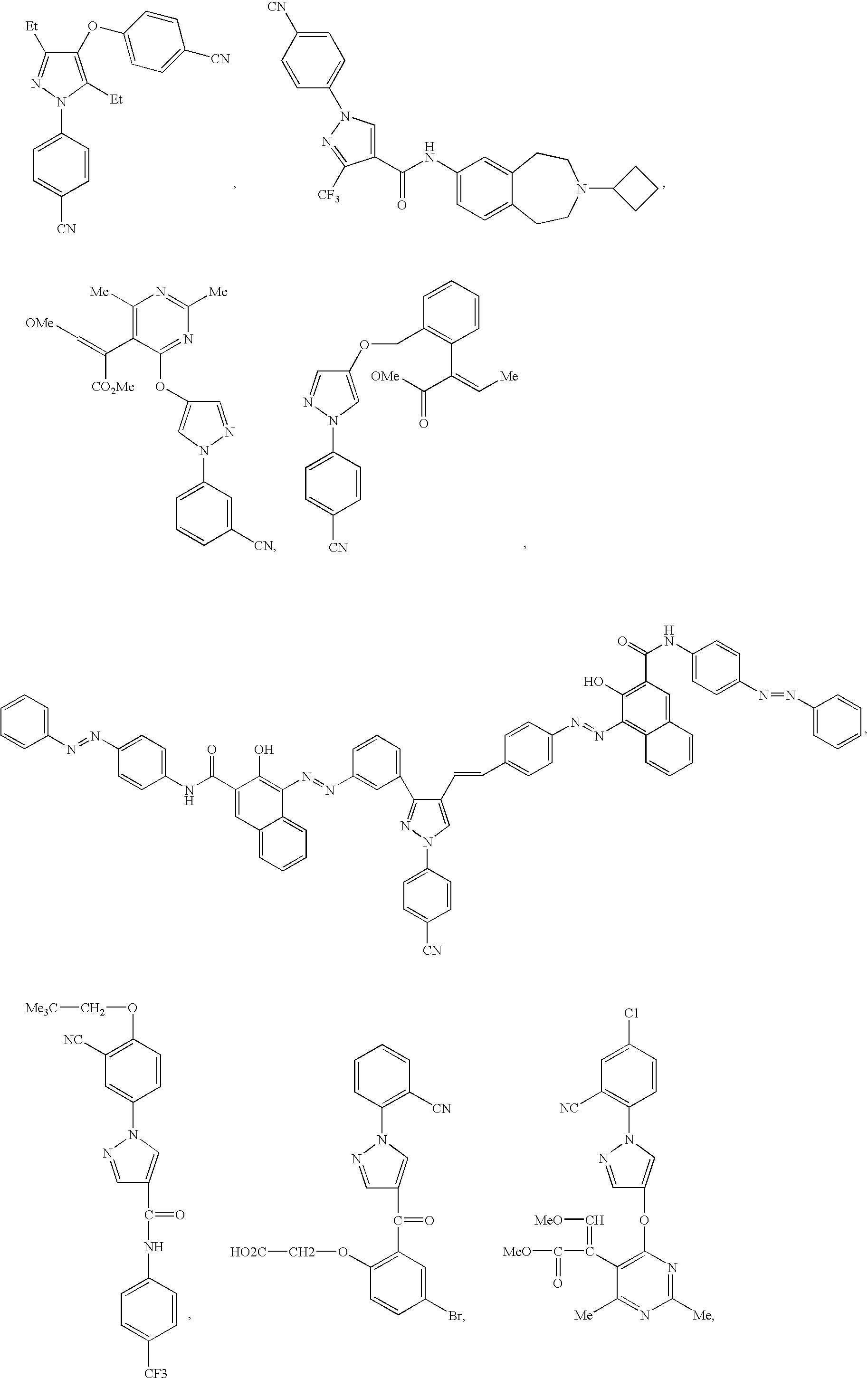

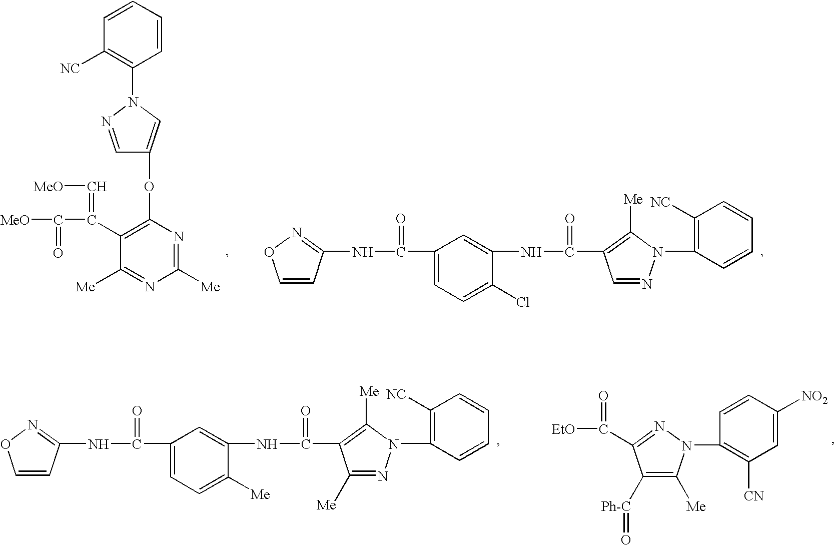

a compound represented by the formula:

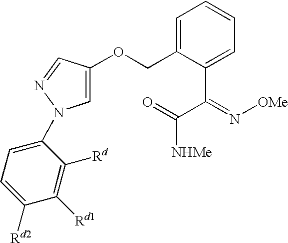

wherein one of R^{d}, R^{d1} and R^{d2} is a cyano group and the other two groups are the same or different and each is hydrogen atom, a chlorine atom or a methyl group, and
a compound represented by the formula:

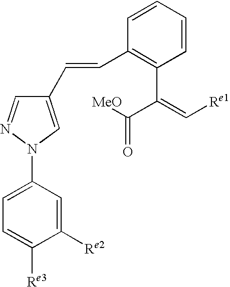

wherein R^{e1} is a methyl group, an ethyl group or a methoxymethyl group, one of R^{e2} and R^{e3} is a cyano group, and the other is a hydrogen atom.

As compound (I), a compound wherein
R¹ is C₁₋₆ alkyl optionally having substituent(s);
R² is phenyl optionally having substituent(s), pyrazolyl optionally having substituent(s), imidazolyl optionally having substituent(s), furyl optionally having substituent(s), isoxazolyl optionally having substituent(s), oxazolyl optionally having substituent(s), pyridyl optionally having substituent(s), thienyl optionally having substituent(s), thiazolyl optionally having substituent(s), pyrimidinyl optionally having substituent(s), indolyl optionally having substituent(s) or quinolyl optionally having substituent(s);
R³ is C₁₋₆ alkyl optionally having substituent(s), C₁₋₆ alkyl-carbonyloxy optionally having substituent(s) or C₁₋₆ alkoxy-carbonyloxy optionally having substituent(s);
R⁴ is 4-cyanophenyl optionally having substituent(s); and
X is —CH₂—, —CH(Me)-, —CH(OH)—, —CH(OCOPh)-, —C(Me)(OH)—, —C(CH₂CH₃)(OH)—, —CO—, —C(═CH₂)—, —O—, —CH₂O—, —CH₂S—, —CH(Me)S—, —CH₂CH₂—, —CH₂SO₂—, (E) —CH═CH—, —NHCO—, —CONH—, —C(Me)(OH)CH₂—, —OCH₂—, —COCONH—, —C(Me)(OH)CONH—, —CONHCH₂—, —CH₂OCH₂—, —COCOCH₂SO₂— or —COCONHCH₂— is preferable.

Particularly, as compound (I), a compound wherein
R¹ is C₁₋₆ alkyl (particularly methyl);
R² is
(A) phenyl optionally having 1 to 3 substituents selected from
(1) a halogen atom (e.g., fluorine atom, chlorine atom, bromine atom),
(2) cyano,
(3) nitro,
(4) C₁₋₆ alkyl (e.g., methyl, tert-butyl) optionally having 1 to 3 substituents selected from (a) a halogen atom (e.g., fluorine atom), (b) cyano and (c) carbamoyl,
(5) C₃₋₆ cycloalkyl (e.g., cyclohexyl),
(6) C₁₋₆ alkoxy (e.g., methoxy) optionally having 1 to 3 halogen atoms (e.g., fluorine atom),
(7) C₆₋₁₀ aryloxy (e.g., phenoxy),
(8) C₁₋₆ alkylthio (e.g., methylthio) optionally having 1 to 3 halogen atoms (e.g., fluorine atom),
(9) C₁₋₆ alkyl-carbonyl (e.g., acetyl),
(10) C₁₋₆ alkyl-sulfonyl (e.g., methylsulfonyl),
(11) C₁₋₆ alkoxy-carbonyl (e.g., methoxycarbonyl),
(12) amino,
(13) di-C₁₋₆ alkylamino (e.g., dimethylamino),
(14) C₁₋₆ alkyl-carbonylamino (e.g., acetylamino),
(15) carbamoyl,
(16) thiocarbamoyl,
(17) carboxy,
(18) C₆₋₁₀ aryl (e.g., phenyl),
(19) C₁₋₆ alkyl-carbamoyl (e.g., methylcarbamoyl, ethylcarbamoyl, propylcarbamoyl, isopropylcarbamoyl, butylcarbamoyl, isobutylcarbamoyl, tert-butylcarbamoyl) optionally having 1 to 3 substituents selected from (a) a halogen atom (e.g., fluorine atom), (b) hydroxy, (c) C₁₋₆ alkoxy (e.g., methoxy, ethoxy) optionally having one hydroxy, (d) C₆₋₁₀ aryl (e.g., phenyl), (e) 5- or 6-membered aromatic heterocyclic group (e.g., furyl, pyridyl), (f) carbamoyl, (g) di-C₁₋₆ alkylamino (e.g., dimethylamino), (h) C₃₋₆ cycloalkyl (e.g., cyclopentyl) optionally having one hydroxy, (i) C₁₋₆ alkylthio (e.g., methylthio), (j) C₁₋₆ alkylsulfinyl (e.g., methylsulfinyl), (k) C₁₋₆ alkylsulfonyl (e.g., methylsulfonyl) and (1) C₁₋₆ alkoxy-carbonyl (e.g., methoxycarbonyl),
(20) C₃₋₆ cycloalkyl-carbamoyl (e.g., cyclopropylcarbamoyl, cyclopentylcarbamoyl),
(21) di-C₁₋₆ alkyl-carbamoyl (e.g., dimethylcarbamoyl, diethylcarbamoyl, N-ethyl-N-methylcarbamoyl) optionally having one substituent selected from C₁₋₆ alkoxy (e.g., methoxy) and hydroxy,
(22) C₆₋₁₀ aryl-carbamoyl (e.g., phenylcarbamoyl) optionally having one C₁₋₆ alkyl (e.g., methyl),
(23) (5- or 6-membered aromatic heterocycle (e.g., thiazolyl, pyridyl)) optionally having one C₁₋₆ alkyl (e.g., methyl) optionally having 1 to 3 halogen atoms (e.g., fluorine atom)-carbamoyl,
(24) cyclic carbamoyl (e.g., azetidinylcarbonyl, pyrrolidinocarbonyl, morpholinocarbonyl, thiomorpholinocarbonyl dioxide, piperazinocarbonyl) optionally having 1 or 2 substituents selected from (a) hydroxy, (b) C₁₋₆ alkyl (e.g., methyl, ethyl) optionally having one hydroxy, (c)carbamoyl, (d) di-C₁₋₆ alkylamino (e.g., dimethylamino), (e) oxo and (f) C₆₋₁₀ aryl (e.g., phenyl),
(25) a 5-membered aromatic heterocyclic group (e.g., pyrazolyl, imidazolyl, triazolyl, oxadiazolyl, thiadiazolyl, isoxazolyl) optionally having one substituent selected from (a) hydroxy, (b) C₁₋₆ alkyl (e.g., methyl, propyl, isopropyl, tert-butyl) optionally having 1 to 3 halogen atoms (e.g., fluorine atom), (c) C₃₋₆ cycloalkyl (e.g., cyclopentyl), (d) cyano and (e) carbamoyl,
(26) a nonaromatic heterocyclic group (e.g., pyrrolidinyl, morpholinyl) optionally having oxo,
(27) an aromatic heterocyclic group (e.g., triazinyl)-amino-carbamoyl optionally having two C₁₋₆ alkoxy (e.g., methoxy),
(28) C₂₋₃ alkyleneoxy (e.g., ethyleneoxy) optionally having 1 or 2 substituents selected from C₁₋₆ alkyl (e.g., methyl) and oxo,
(29) C₁₋₂ alkylenedioxy (e.g., methylenedioxy),
(30) C₂₋₄ alkylene (e.g., trimethylene, tetramethylene) optionally having oxo, and,
(31) C₂₋₄ alkenyleneoxy (e.g., propenyleneoxy) optionally having 1 or 2 substituents selected from C₁₋₆ alkyl (e.g., methyl) and oxo;
(B) pyrazolyl (e.g., 1-pyrazolyl,3-pyrazolyl, 4-pyrazolyl) optionally having 1 to 3 substituents selected from
(1) C₁₋₆ alkyl (e.g., methyl) optionally having 1 to 3 halogen atoms (e.g., fluorine atom), and,
(2) C₆₋₁₀ aryl (e.g., phenyl) optionally having 1 or 2 substituents selected from a halogen atom (e.g., chlorine atom) and cyano;
(C) imidazolyl (e.g., 2-imidazolyl);
(D) furyl (e.g., 3-furyl);
(E) isoxazolyl (e.g., 3-isoxazolyl);
(F) oxazolyl (e.g., 2-oxazolyl);
(G) pyridyl (e.g., 2-pyridyl, 3-pyridyl, 4-pyridyl) optionally having 1 to 3 substituents selected from
(1) a halogen atom (e.g., fluorine atom, bromine atom),
(2) hydroxy,
(3) cyano,
(4) C₁₋₆ alkyl (e.g., methyl) optionally having 1 to 3 halogen atoms (e.g., fluorine atom),
(5) carbamoyl, and,
(6) C₁₋₆ alkyl-carbamoyl (e.g., isobutylcarbamoyl, tert-butylcarbamoyl) optionally having hydroxy;
(H) thienyl (e.g., 3-thienyl);
(I) thiazolyl (e.g., 2-thiazolyl);
(J) pyrimidinyl (e.g., 2-pyrimidinyl) optionally having one halogen atom (e.g., fluorine atom);
(K) indolyl (e.g., 4-indolyl); or
(L) quinolyl (e.g., 8-quinolyl) optionally having one halogen atom (e.g., fluorine atom);
R³ is C₁₋₆ alkyl (particularly methyl), C₁₋₆ alkyl-carbonyloxy (particularly acetoxy) or C₁₋₆ alkoxy-carbonyloxy (particularly methoxycarbonyloxy);
R⁴ is 4-cyanophenyl optionally having one substituent selected from (1) a halogen atom (e.g., fluorine atom, chlorine atom, bromine atom), (2) cyano, (3) C₁₋₆ alkyl (e.g., methyl) optionally having 1 to 3 halogen atoms (e.g., fluorine atom) and (4) C₆₋₁₀ aryl (e.g., phenyl) optionally having. C₁₋₆ alkoxy-carbonyl (e.g., methoxycarbonyl); and
X is —CH₂—, —CH(Me)-, —CH(OH)—, —CH(OCOPh)-, —C(Me)(OH)—, —C(CH₂CH₃)(OH)—, —CO—, —C(═CH₂)—, —O—, —CH₂O—, —CH₂S—, —CH(Me)S—, —CH₂CH₂—, —CH₂SO₂—, (E)-CH═CH—, —NHCO—, —CONH—, —C(Me)(OH)CH₂—, —OCH₂—, —COCONH—, —C(Me)(OH)CONH—, —CONHCH₂—, —CH₂OCH₂—, —COCOCH₂SO₂— or —COCONHCH₂—;
is preferable.

Particularly, as compound (I), a compound wherein
R¹ is methyl;
R² is phenyl optionally having 1 to 3 substituents selected from
(1) C₁₋₆ alkyl-carbamoyl (e.g., methylcarbamoyl, ethylcarbamoyl, propylcarbamoyl, isopropylcarbamoyl, butylcarbamoyl, isobutylcarbamoyl, tert-butylcarbamoyl) optionally having 1 to 3 substituents selected from (a) a halogen atom (e.g., fluorine atom), (b) hydroxy, (c) C₁₋₆ alkoxy (e.g., methoxy, ethoxy) optionally having one hydroxy, (d) C₆₋₁₀ aryl (e.g., phenyl), (e) 5- or 6-membered aromatic heterocyclic group (e.g., furyl, pyridyl), (f) carbamoyl, (g) di-C₁₋₆ alkylamino (e.g., dimethylamino), (h) C₃₋₆ cycloalkyl (e.g., cyclopentyl) optionally having one hydroxy, (i) C₁₋₆ alkylthio (e.g., methylthio), (j) C₁₋₆ alkylsulfinyl (e.g., methylsulfinyl), (k) C₁₋₆ alkylsulfonyl (e.g., methylsulfonyl) and (l) C₁₋₆ alkoxy-carbonyl (e.g., methoxycarbonyl),
(2) cyclic carbamoyl (e.g., azetidinylcarbonyl, pyrrolidinocarbonyl, morpholinocarbonyl, thiomorpholinocarbonyl dioxide, piperazinocarbonyl) optionally having 1 or 2 substituents selected from (a) hydroxy, (b) C₁₋₆ alkyl (e.g., methyl, ethyl) optionally having one hydroxy, (c) carbamoyl, (d) di-C₁₋₆ alkylamino (e.g., dimethylamino), (e) oxo and (f) C₆₋₁₀ aryl (e.g., phenyl), and
(3) a 5-membered aromatic heterocyclic group (e.g., pyrazolyl, imidazolyl, triazolyl, oxadiazolyl, thiadiazolyl, isoxazolyl) optionally having one substituent selected from (a) hydroxy, (b) C₁₋₆ alkyl (e.g., methyl, propyl, isopropyl, tert-butyl) optionally having 1 to 3 halogen atoms (e.g., fluorine atom), (c) C₃₋₆ cycloalkyl (e.g., cyclopentyl), (d) cyano and (e) carbamoyl;
R³ is methyl, acetoxy or methoxycarbonyloxy;
R⁴ is 4-cyanophenyl, 3,4-di-cyanophenyl, 3-bromo-4-cyanophenyl, 3-chloro-4-cyanophenyl, 4-cyano-2-fluorophenyl, 4-cyano-3-fluorophenyl, 4-cyano-3-(trifluoromethyl)phenyl, 4-cyano-2-(trifluoromethyl)phenyl or 4-cyano-3-(4-methoxycarbonylphenyl)phenyl; and
X is —CH₂— or —O— is preferable.

In another embodiment, preferable examples of compound (I) include the above-mentioned compound (Ib), compound (Ic) and compound (Id).

In compound (Ib), R^{b} is a hydrogen atom, a halogen atom, a cyano group, or a C₁₋₆ alkyl group optionally having 1 to 3 halogen atoms.

As R^{b}, a halogen atom or a C₁₋₆ alkyl group optionally having 1 to 3 halogen atoms is preferable, and a halogen atom (e.g., halogen atom) and a trifluoromethyl group are more preferably.

In compound (Ib), X^{b} is —CH₂—, —C₂H₄—, —O—, —O(C₁₋₂ alkylene)-, —CH(OH)—, —OCH(CH₃)—, —OCH(CONH₂)—, —O—CH₂CONH—, —CH(CH₃)—, —CH(OCOC₆H₅)—, —C(CH₃)(OH)—, —C(CH₂CH₃)(OH)—, —CO—, —C(═CH₂)—, —CH₂O—, —CH₂S—, —CH(CH₃)S—, —CH₂SO₂—, —CH═CH—, —NHCO—, —CONH—, —C(CH₃)(OH)CH₂—, —O(CH₂)—, —COCONH—, —C(CH₃)(OH)CONH—, —CONHCH₂—, —CH₂OCH₂—, —COCOCH₂SO₂—, —COCONHCH₂—, —C(CH₃)(OH)CH₂—, and R^{1b} is a C₁₋₆ alkyl group optionally substituted by a hydroxy group or acyl group.

X^{b} is preferably —CH₂—, —C₂H₄— or —O—, and —CH₂— or —O— is more preferable.

In compound (Ib), R^{1b} is a C₁₋₆ alkyl group optionally substituted by a hydroxy group, or acyl group (when X^{b} is —O—, then R^{1b} is a group other than an ethyl group.

R^{1b} is preferably a C₁₋₆ alkyl group, and methyl or ethyl is more preferable.

In compound (Ib), R^{2b} is
(1) an aromatic hydrocarbon group optionally having substituent(s),
(2) a 5- to 7-membered mono-cyclic aromatic heterocyclic group containing 1 to 4 hetero atoms selected from oxygen atom, sulfur atom and nitrogen atom, which optionally has substituent(s), or
(3) a 8- to 12-membered condensed aromatic heterocyclic group containing 1 to 4 hetero atoms selected from oxygen atom, sulfur atom and nitrogen atom, which optionally has substituent(s).

As R^{2b}, a phenyl group optionally having substituent(s), pyridyl group optionally having substituent(s), pyrimidinyl group optionally having substituent(s), pyrazolyl group optionally having substituent(s), imidazolyl group optionally having substituent(s), thiazolyl group optionally having substituent(s), oxazolyl group optionally having substituent(s), oxadiazolyl group optionally having substituent(s), pyrazolyl group optionally having substituent(s), thienyl group optionally having substituent(s), or thiazolyl group optionally having substituent(s) is preferable.

R^{2b} is more preferably
(1) a phenyl group optionally having substituent(s) selected from oxadiazolyl group optionally having substituent(s), pyrazolyl group optionally having substituent(s), carbamoyl group optionally having substituent(s), 2-oxopyrrolidinyl optionally having substituent(s), phenyl group optionally having substituent(s), optionally substituted C₁₋₆ alkyl, optionally substituted C₁₋₆ alkoxy, optionally substituted C₁₋₆ alkylsulfanyl, halogen atom, cyano group, carboxyl group, nitro group, optionally substituted amino and optionally substituted formyl, and acyl group or
(2) a pyridyl group optionally having substituent(s) selected from C₁₋₆ alkyl-carbonylamino group and carbamoyl group optionally having substituent(s), optionally substituted C₁₋₆ alkyl, cyano group, and halogen atom (as the “substituent” that each group other than the “optionally substituted C₁₋₆ alkyl”, “optionally substituted C₁₋₆ alkoxy” and “optionally substituted C₁₋₆ alkylsulfanyl”, those similar to the “substituent” exemplified for the “aromatic ring group optionally having substituent(s)” can be mentioned. As the “substituent” of the “optionally substituted C₁₋₆ alkyl”, “optionally substituted C₁₋₆ alkoxy” and “optionally substituted C₁₋₆ alkylsulfanyl”, substituents selected from substituent group A can be mentioned.

R^{2b} is more preferably a phenyl group or pyridyl group optionally having substituent(s) selected from
(1) an oxadiazolyl group optionally having a carbamoyl group,
(2) a carbamoyl group substituted by a C₁₋₆ alkyl group optionally having a hydroxy group,
(3) a C₁₋₆ alkylcarbonyl-amino group,
(4) a dioxidothiomorpholinocarbonyl group,
(5) a morpholinocarbonyl group,
(6) a piperidinylcarbonyl group optionally having a hydroxy group and
(7) an azetidinylcarbonyl group optionally having a hydroxy group.

R^{2b} is more preferably
(1) a phenyl group optionally having substituent(s) selected from oxadiazolyl group optionally having substituent(s), carbamoyl group optionally having substituent(s), and acyl group, or
(2) a pyridyl group optionally having substituent(s) selected from a C₁₋₆ alkyl-carbonylamino group and carbamoyl group optionally having substituent(s).

In compound (Ib), R^{3b} is a C₁₋₆ alkyl group optionally substituted by a hydroxy group, a C₁₋₆ alkyl-carbonyloxy group or an acyl group, provided that when X^{b} is —O—, R^{3b} is a group other than ethyl group.

R^{3b} is preferably a C₁₋₆ alkyl group, more preferably, methyl or ethyl.

In compound (Ic), X^{c} is —CH₂—, —C₂H₄— or —O—.

X^{c} is preferably —CH₂— or —O—.

In compound (Ic), R^{1c} and R^{3c} are the same or different and each is a C₁₋₆ alkyl group.

R^{1c} and R^{3c} are each preferably methyl or ethyl, more preferably methyl.

In compound (Ic), W is a nitrogen atom or CH.

In compound (Ic), ring A is a benzene ring optionally having 1 to 3 halogen atoms.

The halogen atom on ring A is preferably a fluorine atom or a chlorine atom.

The number of further halogen atom on ring A is preferably 1 or 2, more preferably 1.

In compound (Ic), R^{c} is
(1) an oxadiazolyl group optionally having a carbamoyl group,
(2) a carbamoyl group optionally having a C₁₋₆ alkyl group optionally having a hydroxy group,
(3) a C₁₋₆ alkylcarbonyl-amino group,
(4) a dioxidothiomorpholinocarbonyl group,
(5) a morpholinocarbonyl group,
(6) a piperidinylcarbonyl group optionally having a hydroxy group, or
(7) an azetidinylcarbonyl group optionally having a hydroxy group.

In compound (Id), X^{d} is —O— or —CH₂—.

In compound (Id), W is a nitrogen atom or CH.

In compound (Id), R^{da} is a halogen atom.

R^{da} is preferably a fluorine atom or a chlorine atom.

In compound (Id), R^{1d} and R^{3d} are each a methyl group.

In compound (Id), R^{d} is
(1) an oxadiazolyl group having a carbamoyl group,
(2) a C₁₋₆ alkyl-carbamoyl group optionally having a hydroxy group,
(3) a C₁₋₆ alkylcarbonyl-amino group,
(4) a dioxidothiomorpholinocarbonyl group,
(5) a morpholinocarbonyl group,
(6) a piperidinylcarbonyl group having a hydroxy group, or
(7) an azetidinylcarbonyl group having a hydroxy group.

More specifically, as compound (I), the following compounds or a salt thereof is preferable.
(1) 5-(4-{[1-(3-chloro-4-cyanophenyl)-3,5-dimethyl-1H-pyrazol-4-yl]methyl}phenyl)-1,3,4-oxadiazole-2-carboxamide;
(2) N-tert-butyl-4-{[1-(3-chloro-4-cyanophenyl)-3,5-dimethyl-1H-pyrazol-4-yl]methyl}benzamide;
(3) 2-chloro-4-(4-{4-[(4-hydroxypiperidin-1-yl)carbonyl]benzyl}-3,5-dimethyl-1H-pyrazol-1-yl)benzonitrile;
(4) 4-{[1-(3-chloro-4-cyanophenyl)-3,5-dimethyl-1H-pyrazol-4-yl]methyl}-N-(2-hydroxy-2-methylpropyl)benzamide;
(5) 2-chloro-4-{3,5-dimethyl-4-[4-(morpholin-4-ylcarbonyl)phenoxy]-1H-pyrazol-1-yl}benzonitrile;
(6) 2-chloro-4-(4-{4-[(3-hydroxyazetidin-1-yl)carbonyl]benzyl}-3,5-dimethyl-1H-pyrazol-1-yl)benzonitrile;
(7) 2-chloro-4-(4-{4-[(1,1-dioxidothiomorpholin-4-yl)carbonyl]phenoxy}-3,5-dimethyl-1H-pyrazol-1-yl)benzonitrile; and
(8) N-(6-{[1-(3-chloro-4-cyanophenyl)-3,5-dimethyl-1H-pyrazol-4-yl]oxy}pyridin-3-yl)-2,2-dimethylpropanamide.

In compound (Ia), R^{a} is a hydrogen atom, halogen atom, cyano group, or a C₁₋₆ alkyl group optionally having 1 to 3 halogen atoms.

R^{a} is preferably a halogen atom, or a C₁₋₆ alkyl group optionally having 1 to 3 halogen atoms.

In compound (Ia), X^{a} is C₁₋₃ alkylene, —O—, —O(C₁₋₃ alkylene)-, —CH(OH)—, —OCH(CH₃)—, —OCH(CONH₂)—, —O—CH₂CONH—, —CH(CH₃)—, —CH(OCOC₆H₅)—, —C(CH₃)(OH)—, —C(CH₂CH₃)(OH)—, —CO—, —C(═CH₂)—, —CH₂O—, —CH₂S—, —CH(CH₃)S—, —CH₂SO₂—, —CH═CH—, —NHCO—, —CONH—, —C(CH₃)(OH)CH₂—, —COCONH—, —C(CH₃)(OH)CONH—, —CONHCH₂—, —CH₂OCH₂—, —COCOCH₂SO₂—, —COCONHCH₂—, —C(CH₃)(OH)CH₂—, —O(C₁₋₃alkylene)O—, —OCH₂CON(C₆H₅)— or

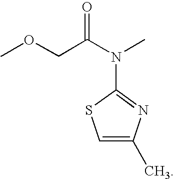

X^{a} is preferably —CH₂—, —C₂H₄— or —O—.

In compound (Ia), R^{1a} is C₁₋₆ alkyl group optionally substituted by a hydroxy group, or an acyl group.

R^{1a} is preferably a C₁₋₆ alkyl group.

In compound (Ia), R^{2a} is
(1) an aromatic hydrocarbon group optionally having substituent(s),
(2) a 5- to 7-membered mono-cyclic aromatic heterocyclic group containing 1 to 4 hetero atoms selected from oxygen atom, sulfur atom and nitrogen atom, which optionally has substituent(s),
(3) a 8- to 12-membered condensed aromatic heterocyclic group containing 1 to 4 hetero atoms selected from oxygen atom, sulfur atom and nitrogen atom, which optionally has substituent(s), or
(4) a nonaromatic heterocyclic group containing 1 to 4 hetero atoms selected from oxygen atom, sulfur atom and nitrogen atom, which optionally has substituent(s).

As R^{2a}, a phenyl group optionally having substituent(s), pyridyl group optionally having substituent(s), pyrimidinyl group optionally having substituent(s), pyrazolyl group optionally having substituent(s), imidazolyl group optionally having substituent(s), thiazolyl group optionally having substituent(s), oxazolyl group optionally having substituent(s), oxadiazolyl group optionally having substituent(s), pyrazolyl group optionally having substituent(s), thienyl group optionally having substituent(s), or thiazolyl group optionally having substituent(s) is preferable.

In compound (Ia), R^{3a} is a C₁₋₆ alkyl group optionally having a hydroxy group, C₁₋₆ alkyl-carbonyloxy group or acyl group. When X^{a} is —O—, then R^{1a} and R^{3a} are groups other than an ethyl group, and R^{2a} is a group other than a cyanophenyl group.

R^{3a} is preferably a C₁₋₆ alkyl group.

More specifically, as compound (Ia), the following compounds or a salt thereof is preferable.
(1) 5-(4-{[1-(3-chloro-4-cyanophenyl)-3,5-dimethyl-1H-pyrazol-4-yl]methyl}phenyl)-1,3,4-oxadiazole-2-carboxamide;
(2) N-tert-butyl-4-{[1-(3-chloro-4-cyanophenyl)-3,5-dimethyl-1H-pyrazol-4-yl]methyl}benzamide;
(3) 2-chloro-4-(4-{4-[(4-hydroxypiperidin-1-yl)carbonyl]benzyl}-3,5-dimethyl-1H-pyrazol-1-yl)benzonitrile;
(4) 4-{[1-(3-chloro-4-cyanophenyl)-3,5-dimethyl-1H-pyrazol-4-yl]methyl}-N-(2-hydroxy-2-methylpropyl)benzamide;
(5) 2-chloro-4-{3,5-dimethyl-4-[4-(morpholin-4-ylcarbonyl)phenoxy]-1H-pyrazol-1-yl}benzonitrile;
(6) 2-chloro-4-(4-{4-[(3-hydroxyazetidin-1-yl)carbonyl]benzyl}-3,5-dimethyl-1H-pyrazol-1-yl)benzonitrile;
(7) 2-chloro-4-(4-{4-[(1,1-dioxidothiomorpholin-4-yl)carbonyl]phenoxy}-3,5-dimethyl-1H-pyrazol-1-yl)benzonitrile; and
(8) N-(6-{[1-(3-chloro-4-cyanophenyl)-3,5-dimethyl-1H-pyrazol-4-yl]oxy}pyridin-3-yl)-2,2-dimethylpropanamide.

As the salt of the compound of the present invention, for example, metal salts, ammonium salts, salts with organic bases, salts with inorganic acids, salts with organic acids, salts with basic or acidic amino acids and the like can be mentioned. As preferable examples of the metal salt, alkali metal salts such as sodium salt, potassium salt and the like; alkaline earth metal salts such as calcium salt, magnesium salt, barium salt and the like; aluminum salt and the like can be mentioned. Preferable examples of the salts with organic bases include salts with trimethylamine, triethylamine, pyridine, picoline, 2,6-lutidine, ethanolamine, diethanolamine, triethanolamine, cyclohexylamine, dicyclohexylamine, N,N′-dibenzylethylenediamine and the like. Preferable examples of the salts with inorganic acids include salts with hydrochloric acid, hydrobromic acid, nitric acid, sulfuric acid, phosphoric acid and the like. Preferable examples of the salts with organic acids include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like. Preferable examples of the salts with basic amino acids include salts with arginine, lysine, ornithine and the like. Preferable examples of the salts with acidic amino acids include salts with aspartic acid, glutamic acid and the like.

Of these, pharmaceutically acceptable salts are preferable. For example, when a compound has an acidic functional group therein, inorganic salts such as alkali metal salts (e.g., sodium salt, potassium salt and the like), alkaline earth metal salts (e.g., calcium salt, magnesium salt, barium salt and the like) and the like, ammonium salt and the like can be mentioned. When a compound has a basic functional group therein, salts with inorganic acids such as hydrochloric acid, hydrobromic acid, nitric acid, sulfuric acid, phosphoric acid and the like, and salts with organic acids such as acetic acid, phthalic acid, fumaric acid, oxalic acid, tartaric acid, maleic acid, citric acid, succinic acid, methanesulfonic acid, p-toluenesulfonic acid and the like can be mentioned.

A prodrug of the compound of the present invention means a compound which is converted to the compound of the present invention by a reaction due to an enzyme, gastric acid, etc. under the physiological conditions in the living body, that is, a compound which is converted to the compound of the present invention by oxidation, reduction, hydrolysis, etc. according to an enzyme; a compound which is converted to the compound of the present invention by hydrolysis etc. due to gastric acid, etc. A prodrug of the compound of the present invention may be a compound obtained by subjecting an amino group in the compound of the present invention to an acylation, alkylation or phosphorylation (e.g., a compound obtained by subjecting an amino group in the compound of the present invention to an eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation and tert-butylation, etc.); a compound obtained by subjecting a hydroxy group in the compound of the present invention to an acylation, alkylation, phosphorylation or boration (e.g., a compound obtained by subjecting an hydroxy group in the compound of the present invention to an acetylation, palmitoylation, propanoylation, pivaloylation, succinylation, fumarylation, alanylation, dimethylaminomethylcarbonylation, etc.); a compound obtained by subjecting a carboxyl group in the compound of the present invention to an esterification or amidation (e.g., a compound obtained by subjecting a carboxyl group in the compound of the present invention to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification and methylamidation, etc.) and the like. Any of these compounds can be produced from the compound of the present invention by a method known per se.

A prodrug of compound (I) of the present invention may also be one which is converted to the compound of the present invention under physiological conditions, such as those described in IYAKUHIN no KAIHATSU (Development of Pharmaceuticals), Vol. 7, Design of Molecules, pp. 163-198, Published by HIROKAWA SHOTEN (1990).

In the following, the production methods of the compound of the present invention or a salt thereof are explained by referring to compound (I) as a typical example. Compounds (I′) and (Ia) can also be produced similarly, or by appropriately modifying the method according to the technique known in the art.

In each production method below, when alkylation reaction, hydrolysis, amination reaction, esterification reaction, amidation reaction, etherification reaction, oxidation reaction, reduction reaction and the like are carried out, these reactions are carried out according to methods known per se. Examples of such method include the methods described in ORGANIC FUNCTIONAL GROUP PREPARATIONS, 2nd edition, ACADEMIC PRESS, INC., 1989; Comprehensive Organic Transformations, VCH Publishers Inc., 1989 etc. and the like.

In addition, the resultant product can be used for the next reaction directly as a reaction mixture or a crude purification product. They can also be isolated from a reaction mixture according to a conventional method, and can be easily produced by a general separation means (e.g., recrystallization, distillation, chromatography and the like).

Compound (I) of the present invention or a salt thereof can be obtained by the method shown in the following reaction scheme 1 or a method analogous thereto and the like, or a known method or a method analogous thereto and the like. A compound represented by the formula (IV) is encompassed in the compound represented by the formula (I). By converting a functional group of the compound of the formula (IV) by a method known per se or a method analogous thereto (e.g., reduction reaction, acylation reaction, sulfonylation reaction, oxidation reaction, alkylation reaction, hydrolysis, amidation reaction, hydrazidation reaction, dehydrating reaction, decarboxylation reaction, dealkoxycarbonylation reaction, sulfuration reaction, trifluoromethylation reaction etc.), a compound encompassed in the formula (I) can also be obtained. When compound (IV) has a protecting group, compound (I) can be obtained by eliminating the protecting group by a method known per se or a method analogous thereto.

Examples of ethers in the production method include diethyl ether, dioxane, tetrahydrofuran and the like.

Examples of saturated hydrocarbons in the production method include hexane, pentane and the like.

Examples of halogenated hydrocarbons in the production method include dichloromethane, chloroform and the like.

Examples of amides in the production method include N,N-dimethylformamide and the like.

Examples of aromatic hydrocarbons in the production method include benzene, toluene and the like.

Examples of alcohols in the production method include methanol, ethanol, 2-propanol and the like.

Examples of ketones in the production method include acetone, methylethylketone and the like.

Examples of nitrites in the production method include acetonitrile and the like.

Examples of esters in the production method include ethyl acetate and the like.

Examples of aromatic amines in the production method include aniline and the like.

Examples of heterocycles in the production method include pyridine and the like.

Examples of organic acids in the production method include formic acid, acetic acid and the like.

Examples of sulfoxides in the production method include dimethyl sulfoxide and the like.

As for each symbol in the compounds in the reaction scheme 1, R²″ is an aromatic ring group optionally having substituent(s), R⁴″ is a phenyl optionally having substituent(s), X¹ is halogen, Y¹ is a hydrogen atom, halogen, a group via a carbon atom, a group via a nitrogen atom, a group via an oxygen atom or a group via a sulfur atom and others are as defined above. Where necessary, they may be protected by protecting groups etc. generally used for organic-synthesis. The compounds in the reaction scheme may form a salt, and as such salt, those similar to the salt of the compound of the present invention and the like can be mentioned.

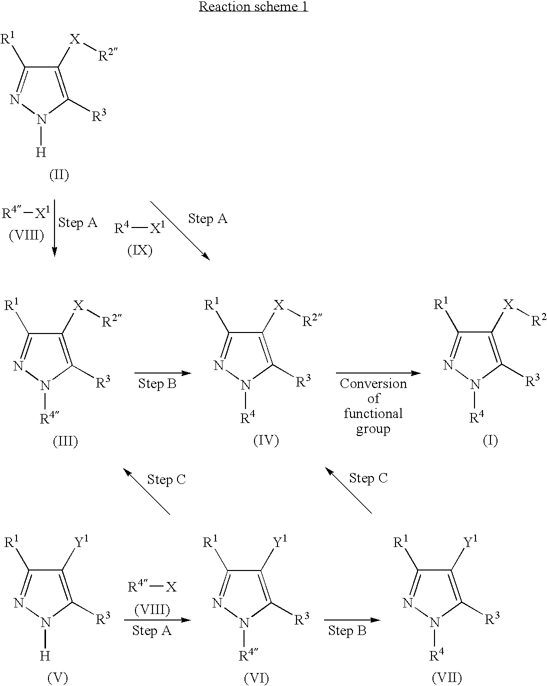

Compounds (II), (III), (V), (VI), (VII), (VIII) and (IX) to be used as starting materials can be produced according to a known method or a method analogous thereto. For example, they can be produced according to method below-mentioned Reference Example. In addition, when they are commercially available, the commercially available products can also be used as they are.

### [Step A]

Compound (II) or (V) is reacted with compound (VIII) or (IX) according to a conventional method in the presence of a base to synthesize compound (III), (IV) or (VI).

Examples of the base include alkali metal hydrides such as sodium hydride, potassium hydride and the like, alkali metal amides such as sodium amide and the like, alkali metal alkoxides such as potassium tert-butoxide and the like, alkali metal carbonates such as potassium carbonate and the like.

The amount of the base to be used is about 1.0 to about 10 mol, preferably about 1.0 to 3.0 mol, per 1.0 mol of compound (II) or (V).

The amount of compound (VIII) or (IX) to be used is about 1.0 to about 10 mol, preferably about 1.0 to 2.0 mol, per 1.0 mol of compound (II) or (V).

The reaction temperature is generally about −70° C. to about 200° C., preferably about 0° C. to about 100° C. The reaction time is generally from about 5 min to about 48 hr, preferably from about 5 min to about 24 hr.

This reaction is generally carried out in an organic solvent that does not adversely influence the reaction. As the organic solvent that does not adversely influence the reaction, for example, ethers, saturated hydrocarbons, halogenated hydrocarbons, amides, aromatic hydrocarbons and the like can be used. These solvents may be used alone or in a mixture of two or more kinds thereof at an appropriate ratio.

When X¹ in compound (VIII) or compound (IX) is chlorine, bromine or iodine, compound (III), compound (IV) or (VI) can be synthesized by a coupling reaction with compound (II) or compound (V) according to a conventional method in the presence of a suitable metal catalyst such as palladium, copper etc.

The reaction can be performed by the method described in a publication (Xie et al., J. Org. Chem. 2006, Vol. 71, pp 6522 or Ma et al., J. Am. Chem. Soc. 1998, Vol. 120, pp 12459), or a method analogous thereto.

The reaction temperature is about 20° C. to about 150° C., preferably about 60° C. to about 120° C. The reaction time is generally about 5 min to about 48 hr, preferably about 5 min to about 24 hr.

[Step B]

Compound (IV) or (VII) can be synthesized by reacting compound (III) or (VI) with a metal cyanide by a method generally used for organic synthesis in the presence of a metal catalyst.

Examples of the metal catalyst include palladium reagents such as tetrakis(triphenylphosphine)palladium and the like, and the like.

Examples of the metal cyanide include zinc cyanide, copper cyanide, sodium cyanide, potassium cyanide and the like.

The amount of metal catalyst to be used is generally 0.01 to 0.2 mol, preferably 0.05 to 0.1 mol, per 1.0 mol of compound (III) or (VI). The amount of the metal cyanide to be used is generally 1 to 20 mol, preferably 2 to 10 mol, per 1.0 mol of compound (III) or (VI).

The reaction time is generally about 5 min to about 50 hr, preferably about 5 min to about 12 hr. The reaction temperature is generally about 0 to about 100° C., preferably about 20 to about 80° C.

This reaction is generally performed in an organic solvent that does not adversely influence the reaction. Examples of the organic solvent that does not adversely influence the reaction include ethers, hydrocarbons, halogenated hydrocarbons, ketones, nitrites, amides, heterocycles and the like. Preferable solvents are ethers, hydrocarbons, halogenated hydrocarbons and amides. These solvents may be used alone or in a mixture of two or more kinds thereof at an appropriately ratio.

[Step C]

When Y¹ of compound (VI) or (VII) is a substituent having carbonyl or formyl, compound (III) or (IV) can be synthesized by reaction with a metal reagent having a corresponding aromatic ring group in a solvent that does not adversely influence the reaction in the presence of an additive such as cerium(III) chloride and the like where necessary.

Examples of the metal reagent include organomagnesium reagents such as alkylmagnesium halide, arylmagnesium halide and the like, organolithium reagents such as alkyllithium, aryllithium and the like, and the like.

Examples of the solvent include hydrocarbons, ethers and the like. These solvents may be used alone or in a mixture of two or more kinds thereof at an appropriately ratio.

The amount of the organomagnesium reagent or organolithium reagent to be used is generally 1 to 20 mol, preferably 1 to 10 mol, per 1.0 mol of compound (VI) or (VII). The amount of additive to be used is generally 0.1 to 10 mol, preferably 1.0 to 5.0 mol, per 1.0 mol of compound (VI) or (VII).

The reaction temperature is generally about −70 to about 100° C., preferably about −70 to about 50° C. The reaction time is generally about 0.5 to about 24 hr.

When Y¹ of compound (VI) or (VII) is a substituent having hydroxy, compound (III) or (IV) can be synthesized by subjecting to the Mitsunobu reaction with an alcohol or a thiol compound having a corresponding aromatic ring group, or a pyrazole compound, in the copresence of a phosphine compound and an azodicarboxylic acid compound, or a phosphorane compound.

Examples of the phosphine compound include triarylphosphines such as triphenylphosphine and the like; trialkylphosphines such as tributylphosphine and the like; alkylarylphosphines such as dicyclohexylphenylphosphine and the like; a resin supported phosphine reagent such as diphenylphosphinopolystyrene resin etc. and the like.

The amount of the phosphine compound to be used is about 1.0 to about 10 mol, preferably about 1.0 to 3.0 mol, per 1.0 mol of compound (VI) or (VII).

Examples of the azodicarboxylic acid compound include azodicarboxylates such as diethyl azodicarboxylate and the like, and azodicarboxamides such as 1,1′-azobis(N,N-dimethylformamide) and the like. As the phosphorane compound, the compound described in a publication (Tsunoda et al., Tetrahedron. Lett. 1996, Vol. 37, pp 2759) can be mentioned.

The amount of the azodicarboxylic acid compound or phosphorane compound to be used is about 1.0 to about 10 mol, preferably about 1.0 to 3.0 mol, per 1.0 mol of compound (VI) or (VII).

The amount of the alcohol or thiol compound or pyrazole compound to be used is about 0.5 to about 10 mol, preferably about 1.0 to 3.0 mol, per 1.0 mol of compound (VI) or (VII).

The reaction temperature is from about 20° C. to about 150° C., preferably from about 60° C. to about 120° C. The reaction time is from about 1 hr to about 50 hr.

This reaction is generally carried out in an organic solvent that does not adversely influence the reaction. As the organic solvent that does not adversely influence the reaction, for example, ethers, saturated hydrocarbons, aromatic hydrocarbons and the like can be used. These solvents may be used alone or in a mixture of two or more kinds thereof at an appropriate ratio.

When Y¹ of compound (VI) or (VII) is a substituent having carboxy, compound (III) or (IV) can be synthesized by activating a carboxylic acid derivative by treatment with an activator and a reaction with aromatic or aliphatic amine in the presence of a base or an additive where necessary, according to a method generally used for organic synthesis.

Examples of the activator include chlorinating agents generally used for organic synthesis such as thionyl chloride, oxalyl chloride and the like, acylating agents generally used for organic synthesis such as acid anhydride, acid chloride and the like, condensation agents generally used for organic synthesis such as 1,3-dicyclohexylcarbodiimide, 1-ethyl-3-(3′-dimethylaminopropyl)carbodiimide, diethylphosphoryl cyanide, N,N-carbonyldiimidazole, 4-(4,6-dimethoxy-1,3,5-triazin-2-yl)-4-methylmorpholin-4-ium chloride and the like, and the like.

Examples of the additive include N-hydroxybenzotriazole, N-hydroxysuccinimide and the like.

Examples of the base include organic bases such as diisopropylethylamine, triethylamine, pyridine and the like, and the like.

The amount of aromatic or aliphatic amine to be used is generally 0.5 to 5.0 mol, preferably 0.8 to 2.0 mol, per 1.0 mol of compound (VI) or (VII). The amount of the activator to be used is generally 1 to 10 mol, preferably 1 to 5 mol, per 1.0 mol of compound (VI) or (VII). The amount of the additive to be used is generally 1 to 20 mol, preferably 2 to 10 mol, per 1.0 mol of compound (VI) or (VII). The amount of the base to be used is generally 1 to 10 mol, preferably 1 to 5 mol, per 1.0 mol of compound (VI) or (VII), or an excess amount thereof can be used as a solvent.

The reaction time is generally about 10 min to about 50 hr, preferably about 30 min to about 12 hr. The reaction temperature is generally about 0 to about 100° C., preferably about 20 to about 80° C.

This reaction is generally performed in an organic solvent that does not adversely influence the reaction. Examples of the organic solvent that does not adversely influence the reaction include ethers, hydrocarbons, halogenated hydrocarbons, ketones, nitrites, amides, heterocycles and the like. Preferable solvent includes ethers, hydrocarbons, halogenated hydrocarbons and amides. These solvents may be used alone or in a mixture of two or more kinds thereof at an appropriately ratio.

When Y¹ of compound (VI) or (VII) is a substituent having chlorocarbonyl, compound (III) or (IV) can be synthesized by reaction with aromatic or aliphatic amine in the presence of a base and an additive where necessary, according to a method generally used for organic synthesis.

Examples of the base include organic bases such as diisopropylethylamine, triethylamine, pyridine and the like, and the like.

The amount of the aromatic or aliphatic amine to be used is generally 0.5 to 5.0 mol, preferably 0.8 to 2.0 mol, per 1.0 mol of compound (VI) or (VII). The amount of the base to be used is generally 1 to 10 mol, preferably 1 to 5 mol, per 1.0 mol of compound (VI) or (VII), or an excess amount thereof can be used as a solvent.

The reaction time is generally about 10 min to about 50 hr, preferably about 30 min to about 12 hr. The reaction temperature is generally about 0 to about 100° C., preferably about 20 to about 80° C.

This reaction is generally performed in an organic solvent that does not adversely influence the reaction. Examples of the organic solvent that does not adversely influence the reaction include ethers, hydrocarbons, halogenated hydrocarbons, ketones, nitrites, amides, heterocycles and the like. Preferable solvent includes ethers, hydrocarbons, halogenated hydrocarbons and amides. These solvents may be used alone or in a mixture of two or more kinds thereof at an appropriately ratio.

When Y¹ of compound (VI) or (VII) is halogen, compound (III) or (IV) can be synthesized by reaction with an olefin compound in the presence of a metal catalyst and a base where necessary, according to a method generally used for organic synthesis.

Examples of the metal catalyst include palladium reagents such as palladium acetate and the like combined as necessary with a suitable ligand such as bis(diphenylphosphino)ferrocene and the like.

Examples of the base include organic bases such as triethylamine, diisopropylethylamine, pyridine and the like, and metal carbonates such as cesium carbonate and the like.

The amount of each of the metal catalyst and ligand to be used is generally 0.01 to 0.5 mol, preferably 0.1 to 0.3 mol, per 1.0 mol of compound (VI) or (VII). The amount of each of the olefin compound and base to be used is generally 1 to 20 mol, preferably 2 to 10 mol, per 1.0 mol of compound (VI) or (VII).

The reaction time is generally about 10 min to about 50 hr, preferably about 30 min to about 12 hr. The reaction temperature is generally about 0 to about 100° C., preferably about 20 to about 80° C.

This reaction is generally performed in an organic solvent that does not adversely influence the reaction. Examples of the organic solvent that does not adversely influence the reaction include ethers, hydrocarbons, halogenated hydrocarbons, ketones, nitrites, amides, heterocycles and the like. Preferable solvent includes ethers, hydrocarbons, halogenated hydrocarbons and amides. These solvents may be used alone or in a mixture of two or more kinds thereof at an appropriately ratio.

When Y¹ of compound (VI) or (VII) is amino, compound (III) or (IV) can be synthesized according to a method generally used for organic synthesis and using an acylating agent such as organic acid, acyl halide, acid anhydride and the like in the presence of a base where necessary.

Examples of the base include alkali metal salts such as sodium hydride, potassium carbonate, sodium carbonate, potassium hydroxide, sodium hydroxide and the like, and organic bases such as triethylamine, diisopropylethylamine, pyridine and the like.

The amount of the acylating agent to be used is generally 1 to 20 mol, preferably 2 to 10 mol, per 1.0 mol of compound (VI) or (VII). The amount of the base to be used is generally 1 to 10 mol, preferably 1 to 5 mol, per 1.0 mol of compound (VI) or (VII). When an organic base such as triethylamine, diisopropylethylamine, pyridine and the like is used, or an excess amount thereof can be used as a solvent.

The reaction temperature is generally 0 to 120° C., preferably 20 to 100° C. The reaction time is generally 0.5 to 100 hr, preferably 1 to 48 hr.

This reaction is generally performed in an organic solvent that does not adversely influence the reaction. Examples of the organic solvent that does not adversely influence the reaction include ethers, saturated hydrocarbons, halogenated hydrocarbons, aromatic hydrocarbons, ketones, nitrites, amides, esters, aromatic amines, heterocycles and the like. Preferable solvents include ethers, hydrocarbons, halogenated hydrocarbons, amides, aromatic amines and heterocycles. These solvents may be used alone or in a mixture of two or more kinds thereof at an appropriately ratio.

When Y¹ of compound (VI) or (VII) is hydrogen, compound (III) or (IV) can be synthesized by a reaction with a corresponding acid chloride in the presence of a Lewis acid such as aluminum chloride, tin chloride and the like according to a method generally used for organic synthesis.

The amount of the Lewis acid to be used is generally 1 to 20 mol, preferably 1 to 10 mol, per 1.0 mol of compound (VI) or (VII). The amount of acid chloride to be used is generally 1 to 10 mol, preferably 1 to 5 mol, per 1.0 mol of compound (VI) or (VII).

The reaction temperature is generally −20 to 120° C., preferably 0 to 100° C. The reaction time is generally 0.5 to 100 hr, preferably 1 to 48 hr.

This reaction is generally performed in an organic solvent that does not adversely influence the reaction. Examples of the organic solvent that does not adversely influence the reaction include saturated hydrocarbons, halogenated hydrocarbons, aromatic hydrocarbons, nitrites can be mentioned. Preferable solvents are hydrocarbons, halogenated hydrocarbons and nitrites. These solvents may be used alone or in a mixture of two or more kinds thereof at an appropriately ratio.

### [Conversion of Functional Group]

The reduction reaction can be carried out using a reducing agent generally used for organic synthesis, such as sodium borohydride, sodium cyanoborohydride, sodium triacetoxyborohydride, lithium borohydride, lithium aluminum hydride, zinc, tin, iron and the like, and a metal salt such as calcium chloride and the like may be added. The amount of the reducing agent to be used is about 1.0 to about 10 mol, preferably about 1.0 to 5.0 mol, per 1.0 mol of compound (IV). The amount of the metal salt to be used is about 0.5 to about 10 mol, preferably about 1.0 to 3.0 mol, per 1.0 mol of compound (IV).

The reaction temperature is generally about −70° C. to about 100° C., preferably about 0° C. to about 50° C. The reaction time is generally from about 30 min to about 50 hr, preferably from 30 min to about 20 hr.

This reaction is generally carried out in an organic solvent that does not adversely influence the reaction. As the organic solvent that does not adversely influence the reaction, for example, alcohols, ethers, saturated hydrocarbons, aromatic hydrocarbons and the like can be used. These solvents may be used alone or in a mixture of two or more kinds thereof at an appropriate ratio.

The acylation reaction can be carried out using an acylating agent such as an organic acid, an acyl halide, an acid anhydride and the like, in the presence of a base where necessary, according to a method generally used for organic synthesis.

As the base, for example, alkali metal salts such as sodium hydride, potassium carbonate, sodium carbonate, potassium hydroxide, sodium hydroxide and the like, and organic bases such as triethylamine, diisopropylethylamine, pyridine and the like can be used.

The amount of the acylating agent to be used is generally 1 to 20 mol, preferably 2 to 10 mol, per 1.0 mol of compound (IV). The amount of the base to be used is generally 1 to 10 mol, preferably 1 to 5 mol, per 1.0 mol of compound (IV). When an organic base (e.g., triethylamine, diisopropylethylamine, pyridine etc.) is used, an excess amount thereof can also be used as a solvent.

The reaction temperature is generally 0 to 120° C., preferably 20 to 100° C. The reaction time is generally 0.5 to 100 hr, preferably 1 to 48 hr.

This reaction is generally carried out in an organic solvent that does not adversely influence the reaction. As the organic solvent that does not adversely influence the reaction, for example, ethers, saturated hydrocarbons, halogenated hydrocarbons, aromatic hydrocarbons, ketones, nitriles, amides, esters, aromatic amines, heterocycles and the like can be used. Preferable examples thereof include ethers, hydrocarbons, halogenated hydrocarbons, amides, aromatic amines and heterocycles. These solvents may be used alone or in a mixture of two or more kinds thereof at an appropriate ratio.

The oxidation reaction can be carried out using an oxidant generally used for organic synthesis, such as manganese compounds (e.g., potassium permanganate, manganese dioxide etc.), chrome compounds (e.g., chromic acid etc.), sulfur compounds (e.g., dimethylsulfoxide etc.), cerium compounds (e.g., cerium (IV) diammonium nitrate etc.), in a solvent that does not adversely influence the reaction, in the presence of an acid, a base and the like where necessary. Examples of the solvent include water, saturated hydrocarbons, aromatic hydrocarbons, halogenated hydrocarbons, alcohols, ketones, organic acids, amides, esters, sulfoxides, nitrites and the like. Preferable examples thereof include water, hydrocarbons, halogenated hydrocarbons, ketones, organic acids, amides, esters, sulfoxides and nitriles. These solvents may be used alone or in a mixture of two or more kinds thereof at an appropriate ratio.

As the acid, for example, mineral acids such as sulfuric acid and the like, organic acids such as acetic acid and the like, and the like can be used. As the base, for example, alkali metal salts such as potassium hydroxide, sodium hydroxide and the like, amines such as triethylamine, diisopropylethylamine, piperidine and the like can be used. Where necessary, a dehydrating agent such as dicyclohexylcarbodiimide and the like, oxalyl chloride, pyridine sulfur trioxide and the like may be added.

The amount of the oxidant to be used is generally 1 to 20 mol, preferably 1 to 10 mol, per 1.0 mol of compound (IV). When dimethylsulfoxide is used, an excess amount thereof can also be used as a solvent. The amount of the acid or base to be used is generally 1 to 20 mol, preferably 1 to 10 mol, per 1.0 mol of compound (IV). The amount of the other additive to be used is generally 1 to 20 mol, preferably 1 to 10 mol, per 1.0 mol of compound (IV).

The reaction temperature is generally −70 to 120° C., preferably −70 to 100° C. The reaction time is generally 0.1 to 100 hr, preferably 0.1 to 48 hr.

The alkylation reaction can be carried out by reacting compound (IV) with an alkylating agent such as alkylhalide and the like in the presence of a base according to a conventional method.

As the base, for example, alkali metal hydride such as sodium hydride, potassium hydride and the like; alkali metal amides such as sodium amide and the like; potassium tert-butoxide, potassium carbonate and the like can be used.

The amount of the base to be used is about 1.0 to about 10 mol, preferably about 1.0 to 2.0 mol, per 1.0 mol of compound (IV).

The amount of the alkylating agent to be used is about 1.0 to about 10 mol, preferably about 1.0 to 2.0 mol, per 1.0 mol of compound (IV).

The reaction temperature is generally about −70° C. to about 100° C., preferably about 0° C. to about 50° C. The reaction time is generally about 5 min to about 48 hr, preferably about 5 min to about 20 hr.

This reaction is generally carried out in an organic solvent that does not adversely influence the reaction. As the organic solvent that does not adversely influence the reaction, for example, ethers, saturated hydrocarbons, halogenated hydrocarbons, amides, aromatic hydrocarbons and the like can be used. These solvents may be used alone or in a mixture of two or more kinds thereof at an appropriate ratio.

The alkylation reaction of a carbonyl group can be carried out by reacting compound (IV) with an alkylating agent in a solvent that does not adversely influence the reaction, in the presence of an additive such as cerium (III) chloride and the like where necessary.

As the alkylating agent, for example, organomagnesium reagents such as alkylmagnesium halide and the like; organolithium reagents such as alkyllithium and the like, and the like can be used.

As the solvent, for example, hydrocarbons, ethers and the like can be used. These solvents may be used alone or in a mixture of two or more kinds thereof at an appropriate ratio.

The amount of the organic magnesium reagent or organolithium reagent to be used is generally 1 to 20 mol, preferably 1 to 10 mol, per 1.0 mol of compound (IV). The amount of the additive to be used is generally 0.1 to 10 mol, preferably 1.0 to 5.0 mol, per 1.0 mol of compound (IV).

The reaction temperature is generally about −70 to about 100° C., preferably about −70 to about 50° C. The reaction time is generally about 0.5 to about 24 hr.

The hydrolysis is carried out using an acid or a base generally used for organic synthesis.

As the acid, for example, mineral acids such as hydrochloric acid and the like; Lewis acids such as boron tribromide and the like; a combination of a Lewis acid and a thiol or a sulfide; organic acids such as trifluoroacetic acid, p-toluenesulfonic acid and the like, and the like can be used.

As the base, for example, metal hydroxides such as sodium hydroxide, potassium hydroxide, barium hydroxide and the like; basic salts such as sodium carbonate, potassium carbonate and the like; metal alkoxides such as sodium methoxide, sodium ethoxide, potassium tert-butoxide and the like; organic bases such as triethylamine, imidazole, formamidine and the like, and the like can be used.

The amount of the acid or base to be used is generally 0.1 to about 50 mol, preferably about 1 to about 20 mol, per 1.0 mol of compound (IV).

This reaction is generally carried out in an organic solvent that does not adversely influence the reaction. As the organic solvent that does not adversely influence the reaction, for example, alcohols, ethers, aromatic hydrocarbons, saturated hydrocarbons, halogenated hydrocarbons, sulfoxides, water or a mixture of two or more kinds thereof and the like can be used.

The reaction time is generally about 10 min to about 50 hr, preferably about 30 min to about 12 hr. The reaction temperature is generally about 0 to about 200° C., preferably about 20 to about 120° C.

The amidation reaction or hydrazidation reaction can be carried out according to a method generally used for organic synthesis, which includes treating a carboxylic acid derivative with an activator to give an activated derivative, and reacting the activated derivative with an aromatic or aliphatic amine, or hydrazine derivative in the presence of a base or an additive where necessary. The reaction can also be carried out by reacting an ester derivative with an aromatic or aliphatic amine in the presence of a base or an additive where necessary.

As the activator, for example, chlorinating agents generally used for organic synthesis, such as thionyl chloride, oxalyl chloride and the like; acylating agents generally used for organic synthesis, such as acid anhydride, acid chloride and the like; condensation agents generally used for organic synthesis, such as 1,3-dicyclohexylcarbodiimide, 1-ethyl-3-(3′-dimethylaminopropyl)carbodiimide, diethylphosphoryl cyanide, N,N-carbonyldiimidazole, 4-(4,6-dimethoxy-1,3,5-triazin-2-yl)-4-methylmorpholin-4-ium chloride and the like, and the like can be used.

As the additive, for example, N-hydroxybenzotriazole, N-hydroxysuccinimide and the like can be used.

As the base, for example, organic bases such as diisopropylethylamine, triethylamine, pyridine and the like, and the like can be used.

The amount of the aromatic or aliphatic amine to be used is generally 0.5 to 5.0 mol, preferably 0.8 to 2.0 mol, per 1.0 mol of compound (IV). The amount of the activator to be used is generally 1 to 10 mol, preferably 1 to 5 mol, per 1.0 mol of compound (IV). The amount of the additive to be used is generally 1 to 20 mol, preferably 2 to 10 mol, per 1.0 mol of compound (IV). The amount of the base to be used is generally 1 to 10 mol, preferably 1 to 5 mol, per 1.0 mol of compound (IV). In addition, an excess amount thereof can also be used as a solvent.

The reaction time is generally about 10 min to about 50 hr, preferably about 30 min to about 12 hr. The reaction temperature is generally about 0 to about 100° C., preferably about 20 to about 80° C.

This reaction is generally carried out in an organic solvent that does not adversely influence the reaction. As the organic solvent that does not adversely influence the reaction, for example, ethers, hydrocarbons, halogenated hydrocarbons, ketones, nitrites, amides, heterocycles and the like can be used. Preferable examples thereof include ethers, hydrocarbons, halogenated hydrocarbons and amides. These solvents may be used alone or in a mixture of two or more kinds thereof at an appropriate ratio.

The amination or amidation reaction can be performed by a coupling reaction with amine or amide compound in the presence of a suitable metal catalyst such as palladium, copper etc. according to a method generally used for organic synthesis.

The reaction can be performed by the method described in a publication (Xie et al., J. Org. Chem. 2006, Vol. 71, pp 6522 or Ma et al., J. Am. Chem. Soc. 1998, Vol. 120, pp 12459), or a method analogous thereto.

The reaction temperature is about 20° C. to about 150° C., preferably about 60° C. to about 120° C. The reaction time is generally about 5 min to about 48 hr, preferably about 5 min to about 24 hr.

The dehydrating reaction or cyclodehydrating reaction can be performed by a method generally used for organic synthesis using a dehydrating agent such as acylhalide, acid anhydride, phosphorus oxychloride and the like in a solvent that does not adversely influence the reaction in the presence of a base where necessary. Examples of the solvent include ethers, hydrocarbons, halogenated hydrocarbons, ketones, nitrites, amides, esters, aromatic amines, heterocycles and the like. Preferable solvent includes ethers, hydrocarbons, halogenated hydrocarbons, amides, aromatic amines and heterocycles. These solvents may be used alone or in a mixture of two or more kinds thereof at an appropriately ratio.

As a base, for example, alkali metal salts such as sodium hydride, potassium carbonate, sodium carbonate and the like, or organic bases such as triethylamine, diisopropylethylamine, pyridine and the like can be used.

The amount of the dehydrating agent to be used is generally 1 to 20 mol, preferably 1 to 10 mol, per 1.0 mol of compound (IV). The amount of the base to be used is generally 1 to 10 mol, preferably 1 to 5 mol, per 1.0 mol of compound (IV), and an excess amount thereof can also be used as a solvent.

The reaction temperature is generally 0 to 120° C., preferably 0 to 100° C. The reaction time is generally 0.5 to 100 hr, preferably 0.5 to 48 hr.

The cyanation reaction can be carried out by reacting aromatic iodide, bromide or chloride with metal cyanide in the presence of, where necessary, a metal catalyst by a method generally used for organic syntheses.

As the metal catalyst, for example, palladium reagents such as tetrakis(triphenylphosphine)palladium and the like can be used.

As the metal cyanide, for example, zinc cyanide, copper cyanide, sodium cyanide, potassium cyanide and the like can be used.

The amount of the metal catalyst to be used is generally 0.01 to 0.2 mol, preferably 0.05 to 0.1 mol, per 1.0 mol of compound (IV). The amount of the metal cyanide to be used is generally 1 to 20 mol, preferably 2 to 10 mol, per 1.0 mol of compound (IV).

The reaction time is generally about 10 min to about 50 hr, preferably about 30 min to about 12 hr. The reaction temperature is generally about 0 to about 100° C., preferably about 20 to about 80° C.

This reaction is generally carried out in an organic solvent that does not adversely influence the reaction. As the organic solvent that does not adversely influence the reaction, for example, ethers, hydrocarbons, halogenated hydrocarbons, ketones, nitrites, amides, heterocycles and the like can be used. Preferable examples thereof include ethers, hydrocarbons, halogenated hydrocarbons and amides. These solvents may be used alone or in a mixture of two or more kinds thereof at an appropriate ratio.

The carbonylation reaction can be carried out by reacting aromatic iodide, bromide or chloride with carbon monoxide in the copresence of a metal catalyst and a base, and where necessary, alcohol such as methanol, ethanol and the like by a method generally used for organic syntheses.

As the metal catalyst, for example, palladium reagents such as palladium acetate and the like can be used in combination with, where necessary, a suitable ligand such as bis(diphenylphosphino)ferrocene and the like.

As the base, for example, organic bases such as triethylamine, diisopropylethylamine, pyridine and the like can be used.

As the carbon monoxide, gaseous carbon monoxide can be used by directly introducing same into the reaction system. In addition, a reagent that produces carbon monoxide in the system, such as molybdenum hexacarbonyl and the like can also be used.

The amount of the metal catalyst and ligand to be used is each generally 0.01 to 0.5 mol, preferably 0.1 to 0.3 mol, per 1.0 mol of compound (IV). The amount of the alcohol and base to be used is each generally 1 to 20 mol, preferably 2 to 10 mol, per 1.0 mol of compound (IV).

The reaction time is generally about 10 min to about 50 hr, preferably about 30 min to about 12 hr. The reaction temperature is generally about 0 to about 100° C., preferably about 20 to about 80° C.

This reaction is generally carried out in an organic solvent that does not adversely influence the reaction. As the organic solvent that does not adversely influence the reaction, for example, ethers, hydrocarbons, halogenated hydrocarbons, ketones, nitriles, amides, heterocycles and the like can be used. Preferable examples thereof include ethers, hydrocarbons, halogenated hydrocarbons and amides. These solvents may be used alone or in a mixture of two or more kinds thereof at an appropriate ratio.

The Curtius rearrangement reaction can be carried out by reacting an aromatic carboxylic acid with an azide compound such as diphenylphosphorylazide and the like in the presence of a base, as necessary in the presence of an alcohol, according to a method generally used for organic synthesis.

As the base, for example, organic bases such as triethylamine, diisopropylethylamine, pyridine and the like can be used. As the alcohol, for example, 2-methyl-2-propanol, ethanol, benzyl alcohol and the like can be used.

The amount of the azide compound to be used is generally 0.5 to 5.0 mol, preferably 1.0 to 2.0 mol, per 1.0 mol of compound (IV). The amount of the base to be used is generally 1.0 to 10 mol, preferably 1.0 to 3.0 mol, per 1.0 mol of compound (IV). The amount of the alcohol to be used is generally 1.0 to 50 mol, preferably 1.0 to 10 mol, per 1.0 mol of compound (IV).

The reaction time is generally about 10 min to about 50 hr, preferably about 30 min to about 12 hr. The reaction temperature is generally about 0 to about 100° C., preferably about 20 to about 80° C.

This reaction is generally carried out in an organic solvent that does not adversely influence the reaction. As the organic solvent that does not adversely influence the reaction, for example, ethers, hydrocarbons, halogenated hydrocarbons, ketones, nitriles, amides, heterocycles and the like can be used. Preferable examples thereof include ethers, hydrocarbons, halogenated hydrocarbons and amides. These solvents may be used alone or in a mixture of two or more kinds thereof at an appropriate ratio.

The sulfuration reaction of compound (IV) is carried out using phosphorus pentasulfide, a Lawesson reagent and the like.

This reaction can be carried out in a solvent that does not adversely influence the reaction. As the solvent, for example, ethers, aromatic hydrocarbons, saturated hydrocarbons, halogenated hydrocarbons or a mixture of two or more kinds thereof and the like can be used.

The amount of the phosphorus pentasulfide, Lawesson reagent and the like to be used is generally 0.5 to 30 mol, preferably 0.5 to 10 mol, per 1.0 mol of compound (IV).

The reaction temperature is generally about 0 to about 150° C., preferably about 20 to about 120° C. The reaction time is generally 10 min to about 50 hr, preferably about 30 min to about 12 hr.

When the protecting group is tert-butyl, triphenylmethyl, tert-butoxycarbonyl, benzyloxycarbonyl, tetrahydropyranyl, 4-methoxybenzyl, 2,4-dimethoxybenzyl and the like, the deprotection can be carried out, for example, by treating compound (IV) with an acid in a solvent that does not adversely influence the reaction. As the solvent, for example, ethers, saturated hydrocarbons, aromatic hydrocarbons, halogenated hydrocarbons, ketones, nitriles, amides, esters, organic acids and the like can be used. Preferable examples thereof include ethers, hydrocarbons and halogenated hydrocarbons. These solvents may be used alone or in a mixture of two or more kinds thereof at an appropriate ratio. As the acid, for example, mineral acids such as hydrochloric acid, hydrobromic acid, sulfuric acid, nitric acid and the like; organic acids such as formic acid, acetic acid, trifluoroacetic acid, p-toluenesulfonic acid and the like; Lewis acids such as boron tribromide and the like; and silica gel can be used. These acids may be used alone or in a mixture of two or more kinds thereof.

The amount of the acid to be used is generally 1 to 100 mol, preferably 1 to 50 mol, per 1.0 mol of compound (IV). In addition, an excess amount thereof can also be used as a solvent.

The reaction temperature is generally −72 to 100° C., preferably 0 to 60° C. The reaction time is generally 0.5 to 100 hr, preferably 0.5 to 48 hr.

When the protecting group is benzyloxycarbonyl, benzyl, benzyloxymethyl and the like, the deprotection can be carried out by subjecting compound (IV) to catalytic hydrogenation reaction. The catalytic hydrogenation reaction is carried out by reacting compound (IV) with hydrogen in the presence of a metal catalyst generally used for organic synthesis, such as palladium-carbon, platinum-carbon and the like. In addition, a mineral acid (e.g., hydrochloric acid etc.), an organic acid (e.g., acetic acid etc.) and the like may be added.

The amount of the metal catalyst to be used is generally about 0.01 to about 1 mol, preferably about 0.01 to 0.5 mol, per 1.0 mol of compound (IV).

The amount of the mineral acid (e.g., hydrochloric acid), organic acid (e.g., acetic acid) and the like to be used is generally about 1.0 to about 50 mol, preferably about 1.0 to 5.0 mol, per 1.0 mol of compound (IV).

The reaction temperature is generally about −10° C. to about 100° C., preferably about 0° C. to about 50° C. The reaction time is generally about 30 min to about 50 hr, preferably 30 min to about 20 hr.

This reaction is generally carried out in an organic solvent that does not adversely influence the reaction. As the organic solvent that does not adversely influence the reaction, for example, alcohols, ethers, saturated hydrocarbons, aromatic hydrocarbons and the like can be used. These solvents may be used alone or in a mixture of two or more kinds thereof at an appropriate ratio.

When the object product is obtained as a free form by the above-mentioned reaction, it can be converted to a salt according to a conventional method. When it is obtained as a salt, it can also be converted to a free form or other salt according to a conventional method.

The compound (I) or a salt thereof obtained by the above-mentioned production methods can be isolated and purified by a known means, such as solvent extraction, liquid conversion, phase transfer, crystallization, recrystallization, chromatography and the like.

When compound (I) or a salt thereof contain an optical isomer, a stereoisomer, a positional isomer or rotational isomer, they are also encompassed in compound (I) or a salt thereof, as well as can be obtained as single products by synthesis methods and separation methods known per se. For example, when compound (I) or a salt thereof contain an optical isomer, an optical isomer resolved from the compound is also encompassed in compound (I) or a salt thereof.

The production methods of the compound of the present invention or a salt thereof have been explained above by referring to compound (I) as a typical example. As mentioned above, compounds (I′) and (Ia) can also be produced in the same manner or by appropriately modifying the method according to the technique known in the art.

Here, the optical isomer can be produced by a method known per se.

The compound or a salt thereof of the present invention may be a solvate or a non-solvate.

The compound or a salt thereof of the present invention may be labeled with an isotope (e.g., ²H, ³H, ¹⁴C, ³⁵S) and the like.

The compound or a salt thereof of the present invention may be a crystal.

The compound or a salt thereof of the present invention may be a cocrystal.

The crystal of the compound or a salt thereof of the present invention (hereinafter sometimes to be abbreviated as the crystal of the present invention) can be produced by crystallization of the compound or a salt thereof of the present invention by applying a crystallization method known per se.

Since the compound or a salt thereof of the present invention show superior androgen receptor antagonistic action and the like, low toxicity and a fewer side effects, they are useful as safe pharmaceutical products, androgen receptor antagonists and the like.

Since a pharmaceutical composition containing the compound or a salt thereof of the present invention show a superior androgen receptor antagonistic action and/or a prostate specific antigen (PSA) production inhibitory action in mammals (e.g., mouse, rat, hamster, rabbit, cat, dog, bovine, sheep, monkey, human and the like), and superior (oral) absorbability, superior (metabolism) stability and the like, it can be used an agent for the prophylaxis or treatment of androgen receptor-associated diseases, for example, hormone sensitive diseases in the androgen-dependent phase and/or the androgen-independent phase, particularly hormone sensitive cancer in the androgen-dependent phase and/or the androgen-independent phase (e.g., prostate cancer (e.g., hormone-dependent prostate cancer, hormone independent prostate cancer etc.), uterine cancer, breast cancer (including progressive breast cancer, for example, invasive ductal carcinoma, non-invasive ductal carcinoma, inflammatory breast cancer etc.), pituitary gland tumor, liver cancer (e.g., primary liver cancer, extrahepatic bile duct cancer etc.) and the like), and sex hormone sensitive diseases such as prostatomegaly, endometriosis, hysteromyoma, early puberty, dysmenorrhea, amenorrhea, premenstrual syndrome, polycystic ovary syndrome and the like, contraceptive (or agent for the prophylaxis or treatment of infertility when the rebound effect after cessation of the drug is utilized) and the like.

Particularly, since the compound or a salt thereof of the present invention show an antagonistic action on normal androgen receptor and/or mutant receptor, they can exhibit a superior prophylactic or therapeutic effect on hormone sensitive cancers in the androgen-dependent phase and/or the androgen-independent phase.

Of the androgen receptor antagonists, a drug showing an antagonistic action on mutant androgen receptors and a drug showing an antagonistic action on androgen receptors with enhanced sensitivity are also useful as agents for the prophylaxis or treatment of hormone sensitive cancers in the androgen-dependent phase and/or the androgen-independent phase.

A pharmaceutical agent containing the compound or a salt thereof of the present invention can be safely administered orally or parenterally (e.g., topical, rectal, intravenous administration etc.), for example, after admixing the androgen receptor antagonist of the present invention with a pharmacologically acceptable carrier to give a pharmaceutical composition such as tablets (including sugar-coated tablets and film-coated tablets), powders, granules, capsules (including soft capsules), liquids, injections, suppositories, sustained-release agents and the like, according to a method known per se. Injection can be administered by intravenous, intramuscular, subcutaneous or intraorgan administration or directly to the lesion.

Examples of the pharmacologically acceptable carrier that can be used for the production of the pharmaceutical agent of the present invention include various organic or inorganic carriers conventionally used as preparation materials. For example, excipient, lubricant, binder and disintegrant for solid preparations, or solvent, solubilizing agents, suspending agent, isotonic agent, buffer, soothing agent and the like for liquid preparations can be mentioned. Furthermore, where necessary, suitable amount of conventional preservative, antioxidant, colorant, sweetening agent, adsorbent, wetting agent and the like can be used as appropriate.

Examples of the excipient include lactose, sucrose, D-mannitol, starch, cornstarch, crystalline cellulose, light anhydrous silicic acid and the like.

Examples of the lubricant include magnesium stearate, calcium stearate, talc, colloidal silica and the like.

Examples of the binder include crystalline cellulose, sucrose, D-mannitol, dextrin, hydroxypropylcellulose, hydroxypropylmethylcellulose, polyvinylpyrrolidone, starch, sucrose, gelatin, methylcellulose, sodium carboxymethylcellulose and the like.

Examples of the disintegrant include starch, carboxymethylcellulose, carboxymethylcellulose calcium, sodium carboxymethylstarch, L-hydroxypropylcellulose and the like.

Examples of the solvent include water for injection, alcohol, propylene glycol, macrogol, sesame oil, corn oil, olive oil and the like.

Examples of the solubilizing agent include polyethylene glycol, propylene glycol, D-mannitol, benzyl benzoate, ethanol, trisaminomethane, cholesterol, triethanolamine, sodium carbonate, sodium citrate and the like.

Examples of the suspending agent include surfactants such as stearyl triethanolamine, sodium laurylsulfate, laurylaminopropionic acid, lecithin, benzalkonium chloride, benzethonium chloride, glycerin monostearate, etc.; hydrophilic polymers such as polyvinyl alcohol, polyvinylpyrrolidone, sodium carboxymethyl cellulose, methyl cellulose, hydroxymethyl cellulose, hydroxyethyl cellulose, hydroxypropyl cellulose and the like.

Examples of the isotonic agent include glucose, D-sorbitol, sodium chloride, glycerol, D-mannitol and the like.

Examples of the buffer include a buffer solution of phosphate, acetate, carbonate, citrate and the like.

Examples of the soothing agent include benzyl alcohol and the like.

Examples of the preservative include paraoxybenzoates, chlorobutanol, benzyl alcohol, phenethyl alcohol, dehydroacetic acid, sorbic acid and the like.

Examples of the antioxidant include sulfite, ascorbic acid, α-tocopherol and the like.

The content of the compound or a salt thereof of the present invention in the pharmaceutical agent of the present invention can be appropriately determined in consideration of the administration subject, administration route, diseases and the like. For example, it is usually from about 0.01 to 100% by weight, preferably from about 0.1 to 50% by weight, further preferably from about 0.5 to 20% by weight, based on the whole preparation, though subject to variation depending on the form of the preparation.

While the content of additives such as a carrier and the like in the pharmaceutical composition of the present invention varies depending on the form of the preparation, it is usually from about 1 to 99.99% by weight, preferably from about 10 to 90% by weight, based on the whole preparation.

The compound or a salt thereof of the present invention are low toxic and can be used safely. The daily dose thereof varies depending on the kind of the compound, age, body weight and symptom of patients, dosage form, administration method and the like. For intravenous administration to a patient for, for example, the purpose of treating prostate cancer, the daily dose for an adult (body weight about 60 kg) is about 0.01 to about 1000 mg/kg, preferably about 0.01 to about 100 mg/kg, more preferably about 0.1 to about 100 mg/kg, particularly about 0.1 to about 50 mg/kg, especially about 1.5 to about 30 mg/kg, which is intravenously administered once a day or in several portions a day. It is needless to say that the dose varies depending on various conditions as mentioned above. Therefore, a dose smaller than the aforementioned dose may be sufficient or an excess dose may be necessary in some cases.

As a drug that can be concurrently used along with the compound or a salt thereof of the present invention, for example, hormonal therapeutic agent, anti-cancer agent (e.g., chemotherapeutic agent, immunotherapeutic agent (including vaccine), antibody, gene therapy drug, pharmaceutical agent that inhibits the action of a cell growth factor and receptor thereof, pharmaceutical agent that inhibits angiogenesis) and the like (hereinafter to be abbreviated as concomitant drug) can be used.

Although the compound or a salt thereof in the present invention exhibits excellent anticancer action even when used as a simple agent, its effect can be enhanced by using it in combination with one or more of the combination drug(s) mentioned above (multi-agent co-administration).

As examples of the “hormonal therapeutic agents”, there can be used fosfestrol, diethylstylbestrol, chlorotrianisene, medroxyprogesterone acetate, megestrol acetate, chlormadinone acetate, cyproterone acetate, danazol, dienogest, asoprisnil, allylestrenol, gestrinone, nomegestrol, tadenan, mepartricin, raloxifene, ormeloxifene, levormeloxifene, anti-estrogens (e.g., tamoxifen citrate, toremifene citrate etc.), ER down-regulator (e.g., fulvestrant etc.), human postmenopausal gonadotropin, follitropin, pill preparations, mepitiostane, testrolactone, aminoglutethimide, LH-RH derivative (e.g., LH-RH agonists (e.g., goserelin acetate, buserelin, leuprorelin etc.), LH-RH antagonist), droloxifene, epitiostanol, ethinylestradiol sulfonate, aromatase inhibitors (e.g., fadrozole hydrochloride, anastrozole, retrozole, exemestane, vorozole, formestane etc.), anti-androgens (e.g., flutamide, bicartamide, nilutamide etc.), 5α-reductase inhibitors (e.g., finasteride, dutasteride, epristeride etc.), adrenocorticohormone drugs (e.g., dexamethasone, prednisolone, betamethasone, triamcinolone etc.), androgen synthesis inhibitors (e.g., abiraterone etc.), retinoid and drugs that retard retinoid metabolism (e.g., liarozole etc.), and the like.

As examples of the “chemotherapeutic agents”, there can be used alkylating agents, antimetabolites, anticancer antibiotics, plant-derived anticancer agents, and the other chemotherapeutic agents.

The “alkylating agents” include nitrogen mustard, nitrogen mustard N-oxide hydrochloride, chlorambucil, cyclophosphamide, ifosfamide, thiotepa, carboquone, improsulfan tosylate, busulfan, nimustine hydrochloride, mitobronitol, melphalan, dacarbazine, ranimustine, sodium estramustine phosphate, triethylenemelamine, carmustine, lomustine, streptozocin, pipobroman, etoglucid, carboplatin, cisplatin, miboplatin, nedaplatin, oxaliplatin, altretamine, ambamustine, dibrospidium hydrochloride, fotemustine, prednimustine, pumitepa, ribomustin, temozolomide, treosulphan, trophosphamide, zinostatin stimalamer, adozelesin, cystemustine, bizelesin etc.

The “antimetabolites” include mercaptopurine, 6-mercaptopurine riboside, thioinosine, methotrexate, enocitabine, cytarabine, cytarabine ocfosfate, ancitabine hydrochloride, 5-FU drugs (e.g., fluorouracil, tegafur, UFT, doxifluridine, carmofur, gallocitabine, emitefur, etc.), aminopterine, leucovorin calcium, tabloid, butocine, folinate calcium, levofolinate calcium, cladribine, emitefur, fludarabine, gemcitabine, hydroxycarbamide, pentostatin, piritrexim, idoxuridine, mitoguazone, thiazophrine, and ambamustine, etc.

The “anticancer antibiotics” include actinomycin-D, actinomycin-C, mitomycin-C, chromomycin-A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride, etc.

The “plant-derived anticancer agents” include etoposide, etoposide phosphate, vinblastine sulfate, vincristine sulfate, vindesine sulfate, teniposide, paclitaxel, docetaxel, vinorelbine, irinotecan, topotecan etc.

As “other chemotherapeutic agent”, for example, sobuzoxane and the like can be used.

The “immunotherapeutic agents (BRM)” include picibanil, krestin, sizofiran, lentinan, ubenimex, interferons, interleukins, macrophage colony-stimulating factor, granulocyte colony-stimulating factor, erythropoietin, lymphotoxin, Corynebacterium parvum, levamisole, polysaccharide K, procodazole, etc. As the vaccine, BCG vaccine, PROVENGE, Onyvax-P, PROSTVAC-VF, GVAX, DCVax-Prostate, SAPOIMMUNE, VPM-4-001 and the like can be used.

As the “antibody”, an antibody to EpiCAM, an antibody to PSCA, an antibody to PSMA and the like are used.

The “cell growth factor” in the “drugs that inhibit the activity of cell growth factors or cell growth factor receptors” includes any substances that promote cell proliferation, which are normally peptides having a molecular weight of not more than 20,000 that are capable of exhibiting their activity at low concentrations by binding to a receptor, including (1) EGF (epidermal growth factor) or substances possessing substantially the same activity as it [e.g., EGF, heregulin, TGF-α, HB-EGF etc.], (2) insulin or substances possessing substantially the same activity as it [e.g., insulin, IGF (insulin-like growth factor)-1, IGF-2, etc.], (3) FGF (fibroblast growth factor) or substances possessing substantially the same activity as it [e.g., acidic FGF, basic FGF, KGF (keratinocyte growth factor), FGF-10, etc.], (4) other cell proliferation factors [e.g., CSF (colony stimulating factor), EPO (erythropoietin), IL-2 (interleukin-2), NGF (nerve growth factor), PDGF (platelet-derived growth factor), TGF β (transforming growth factor β), HGF (hepatocyte growth factor), VEGF (vascular endothelial growth factor), etc.], etc.

The “cell growth factor receptors” include any receptors capable of binding to the aforementioned cell growth factors and, specifically, EGF receptor and HER2, HER3 and HER4, which are the receptors belonging to the same family, insulin receptor, IGF receptor, FGF receptor-1, FGF receptor-2 and the like can be mentioned.

The “drugs that inhibit the activity of cell proliferation factor” include trastuzumab (Herceptin (trademark): (HER2 antibody)), imatinib mesylate, ZD1839, cetuximab, gefitinib, erlotinib and the like.

As the “pharmaceutical agent that inhibits angiogenesis”, antibody to VEGF (e.g., bevacizumab), antibody to VEGF receptor, VEGF receptor kinase inhibitor (e.g., SU11248 etc.), PDGF receptor kinase inhibitor, Tie2 kinase inhibitor, thalidomide and the like can be used.

In addition to the aforementioned drugs, L-asparaginase, aceglatone, procarbazine hydrochloride, protoporphyrin-cobalt complex salt, mercuric hematoporphyrin-sodium, differentiation inducers (e.g., retinoid, vitamin D, etc.), α-blockers (e.g., tamsulosin hydrochloride, naftopidil, urapidil, alfuzosin, terazosin, prazosin, silodosin etc.) serine/threonine kinase inhibitor, endothelin receptor antagonist (e.g., atrasentan etc.), proteasome inhibitor (e.g., bortezomib etc.), Hsp 90 inhibitor (e.g., 17-AAG etc.), spironolactone, minoxidil, 11α-hydroxyprogesterone, bone resorption inhibitory metastasis suppressing agent (e.g., zoledronic acid, alendronic acid, pamidronic acid, etidronic acid, ibandronic acid, clodronic acid) and the like can be used.

Particularly preferable concomitant drug is, for example, LH-RH derivative and the like.

The LH-RH derivative includes an LH-RH derivative or salt thereof which is effective against hormone-dependent diseases, especially sex hormone-dependent diseases such as sex hormone-dependent cancers (e.g., prostate cancer, uterine cancer, breast cancer, hypophyseal tumor, liver cancer, etc.), prostatic hypertrophy, endometriosis, uterine myoma, precocious puberty, dysmenorrhea, amenorrhea, premenstrual syndrome, multilocular ovary syndrome, etc., and contraception (or infertility when rebound effect after drug withdrawal is applied). Further it includes an LH-RH derivative or salt thereof which is effective against benign tumor or malignant tumor which is sex hormone-independent and LH-RH sensitive.

Specific examples of the LH-RH derivatives or salt thereof include peptides described in “Treatment with GnRH analogs: Controversies and perspectives” issued in 1996 by The Parthenon Publishing Group Ltd., PCT Japanese Translation Patent Publication No. 3-503165, JP-A 3-101695, JP-A 7-97334 and JP-A 8-259460, etc.

The LH-RH derivative includes LH-RH agonists and LH-RH antagonists. The LH-RH antagonist includes, for example, a physiologically active peptide represented by the formula:

X-D2Nal-D4ClPhe-D3Pal-Ser-A-B-Leu-C-Pro-DAlaNH₂

[wherein X is N(4H₂-furoyl)Gly or NAc, A is a residue selected from NMeTyr, Tyr, Aph(Atz) and NMeAph(Atz), B is a residue selected from DLys(Nic), DCit, DLys(AzaglyNic), DLys(AzaglyFur), DhArg(Et₂), DAph(Atz) and DhCi, and C is Lys(Nisp), Arg or hArg(Et₂)] or a salt thereof, etc.

The LH-RH agonist includes, for example, a physiologically active peptide represented by the formula:

5-oxo-Pro-His-Trp-Ser-Tyr-Y-Leu-Arg-Pro-Z

[wherein Y is a residue selected from DLeu, DAla, DTrp, DSer(tBu), D2Nal and DHis(ImBzl) and Z is NH—C₂H₅ or Gly-NH₂] or a salt thereof, etc, especially, suitably, a peptide wherein Y is DLeu, and Z is NH—C₂H₅ (that is, Peptide A represented by 5-oxo-Pro-His-Trp-Ser-Tyr-DLeu-Leu-Arg-Pro-NH—C₂H₅; leuprorelin) or a salt thereof (e.g., acetate).

A pharmaceutical agent containing the compound or a salt thereof of the present invention and a concomitant drug in combination (hereinafter a combination drug of the present invention) is low toxic and can be safely administered orally or parenterally (e.g., topical, rectal, intravenous administration etc.), for example, after admixing the androgen receptor antagonist of the present invention and/or the aforementioned concomitant drug with a pharmacologically acceptable carrier to give a pharmaceutical composition such as tablets (including sugar-coated tablets and film-coated tablets), powders, granules, capsules (including soft capsules), liquids, injections, suppositories, sustained-release agents and the like, according to a method known per se. Injection can be administered by intravenous, intramuscular, subcutaneous or intraorgan administration or directly to the lesion.

Examples of the pharmacologically acceptable carrier that can be used for the production of the combination drug in the present invention include various organic or inorganic carriers conventionally used as preparation materials. For example, excipient, lubricant, binder and disintegrant for solid preparations, or solvent, solubilizing agents, suspending agent, isotonic agent, buffer, soothing agent and the like for liquid preparations can be mentioned. Furthermore, where necessary, suitable amount of conventional preservative, antioxidant, colorant, sweetening agent, adsorbent, wetting agent and the like can be used as appropriate.

Examples of the excipient include lactose, sucrose, D-mannitol, starch, cornstarch, crystalline cellulose, light anhydrous silicic acid and the like.

Examples of the lubricant include magnesium stearate, calcium stearate, talc, colloidal silica and the like.

Examples of the binder include crystalline cellulose, sucrose, D-mannitol, dextrin, hydroxypropylcellulose, hydroxypropylmethylcellulose, polyvinylpyrrolidone, starch, sucrose, gelatin, methylcellulose, sodium carboxymethylcellulose and the like.

Examples of the disintegrant include starch, carboxymethylcellulose, calcium carboxymethylcellulose, sodium carboxymethyl starch, L-hydroxypropylcellulose and the like.

Examples of the solvent include water for injection, alcohol, propylene glycol, macrogol, sesame oil, corn oil, olive oil and the like.

Examples of the solubilizing agents include polyethylene glycol, propylene glycol, D-mannitol, benzyl benzoate, ethanol, trisaminomethane, cholesterol, triethanolamine, sodium carbonate, sodium citrate and the like.

Example of the suspending agent include a surfactant such as stearyltriethanolamine, sodium lauryl sulfate, lauryl aminopropionic acid, lecithin, benzalkonium chloride, benzethonium chloride, glycerol monostearate and the like; a hydrophilic polymer such as polyvinyl alcohol, polyvinylpyrrolidone, sodium carboxymethylcellulose, methylcellulose, hydroxymethylcellulose, hydroxyethylcellulose, hydroxypropylcellulose etc. and the like.

Examples of the isotonic agent include glucose, D-sorbitol, sodium chloride, glycerol, D-mannitol and the like.

Examples of the buffer include a buffer solution of phosphate, acetate, carbonate, citrate etc. and the like.

Examples of the soothing agent include benzyl alcohol and the like.

Examples of the preservative include paraoxybenzoates, chlorobutanol, benzyl alcohol, phenethyl alcohol, dehydroacetic acid, sorbic acid and the like.

Examples of the antioxidant include sulfite, ascorbic acid, α-tocopherol and the like.

The mixing ratio of the androgen receptor antagonist of the present invention and a concomitant drug in the combination drug in the present invention can be appropriately determined according to the subject of administration, administration route, disease and the like.

For example, the content of the androgen receptor antagonists of the present invention in the combination drug of the present invention varies depending on the form of preparation, and is usually from about 0.01% by weight to 99.9% by weight, preferably from about 0.1% by weight to 50% by weight, more preferably from about 0.5% by weight to 20% by weight, relative to the total of the preparation.

The content of the concomitant drug in the combination drug in the present invention varies depending on the form of preparation, and is usually from about 0.01% by weight to 99.9% by weight, preferably from about 0.1% by weight to 50% by weight, more preferably from about 0.5% by weight to 20% by weight, relative to the total of the preparation.

The content of additives such as a carrier etc. in the combination drug of the present invention varies depending on the form of preparation, and is usually from about 1% by weight to 99.98% by weight, preferably from about 10% by weight to 90% by weight, to the total of the preparation.

When the androgen receptor antagonist of the present invention and the concomitant drug are formulated separately, the same contents may be adopted.

These preparations can be manufactured by a per se known method commonly used in the pharmaceutical manufacturing process.

For example, the androgen receptor antagonists of the present invention and the combination drug can be made as an injection such as an aqueous injection together with a dispersing agent (e.g., Tween 80 (manufactured by Atlas Powder, US), HCO 60 (manufactured by Nikko Chemicals Co., Ltd.), polyethylene glycol, carboxymethyl cellulose, sodium alginate, hydroxypropylmethyl cellulose, dextrin etc.), a stabilizer (e.g., ascorbic acid, sodium pyrosulfite, etc.), a surfactant (e.g., Polysorbate 80, macrogol etc.), a solubilizer (e.g., glycerin, ethanol etc.), a buffer (e.g., phosphoric acid and alkali metal salt thereof, citric acid and alkali metal salt thereof, etc.), an isotonizing agent (e.g., sodium chloride, potassium chloride, mannitol, sorbitol, glucose etc.), a pH regulator (e.g., hydrochloric acid, sodium hydroxide etc.), an antiseptic (e.g., ethyl paraoxybenzoate, benzoic acid, methylparaben, propylparaben, benzyl alcohol etc.), a dissolving agent (e.g., conc. glycerin, meglumine etc.), a solubilizing agent (e.g., propylene glycol, sucrose etc.), a soothing agent (e.g., glucose, benzyl alcohol etc.), etc., or an oily injection by dissolving, suspending or emulsifying them in a vegetable oil such as olive oil, sesame oil, cotton seed oil, corn oil etc. or a solubilizing agent such as propylene glycol, and processing them.

In the case of a preparation for oral administration, the androgen receptor antagonists of the present invention and the combination drug can be made as a preparation for oral administration by adding an excipient (e.g., lactose, sucrose, starch etc.), a disintegrating agent (e.g., starch, calcium carbonate etc.), a binder (e.g., starch, arabic gum, carboxymethyl cellulose, polyvinylpyrrolidone, hydroxypropyl cellulose etc.), a lubricant (e.g., talc, magnesium stearate, polyethylene glycol 6000 etc.) etc., to the compound of the present invention or the combination drug, according to a per se known method, and compressing and molding the mixture, then if desired, coating the molded product by a per se known method for the purpose of masking of taste, enteric property or sustained release. The film forming agent includes, for example, hydroxypropylmethyl cellulose, ethyl cellulose, hydroxymethyl cellulose, hydroxypropyl cellulose, polyoxyethylene glycol, Tween 80, Pluronic F68, cellulose acetate phthalate, hydroxypropylmethyl cellulose phthalate, hydroxymethyl cellulose acetate succinate, Eudragit (methacrylic acid/acrylic acid copolymer, manufactured by Rohm, DE), pigment (e.g., iron oxide red, titanium dioxide, etc.) etc. The preparation for oral administration may be either a rapid release preparation or a sustained release preparation.

For example, in the case of a suppository, the androgen receptor antagonists of the present invention and the combination drug can be made into an oily or aqueous solid, semisolid or liquid suppository according to a per se known method. The oily base used in the above-mentioned composition includes, for example, glycerides of higher fatty acids [e.g., cacao butter, Witepsols (manufactured by Dynamite Nobel, DE), etc.], middle-chain fatty acids [e.g., Miglyols (manufactured by Dynamite Nobel, DE), etc.], or vegetable oils (e.g., sesame oil, soy bean oil, cotton seed oil etc.), etc. Further, the aqueous base includes, for example, polyethylene glycols and propylene glycol, and the aqueous gel base includes, for example, natural gums, cellulose derivatives, vinyl polymers, acrylic acid polymers, etc.

The above-mentioned sustained release agent includes sustained release microcapsules, etc.

For obtaining a sustained release microcapsule, a per se known method can be adopted. For example, it is preferable to mold into a sustained release preparation shown in [2] below.

The androgen receptor antagonist of the present invention is preferably molded into an oral administration preparation such as a solid preparation (e.g., powder, granule, tablet, capsule, etc.) etc., or molded into a rectal administration preparation such as a suppository. Particularly, an oral administration preparation is preferable.

The combination drug can be made into the above-mentioned drug form depending on the kind of the drug.

In the following, there will be shown specifically [1] an injection of the compound or a salt thereof in the present invention or the combination drug and preparation thereof, [2] a rapid release preparation or sustained release preparation of the compound or a salt thereof in the present invention or the combination drug and preparation thereof and [3] a sublingual tablet, a buccal or an intraoral quick integrating agent of the compound or a salt thereof in the present invention or the combination drug and preparation thereof.

### [1] Injection and Preparation Thereof

It is preferred that an injection is prepared by dissolving the compound or a salt thereof in the present invention or the combination drug in water. This injection may be allowed to contain a benzoate and/or a salicylate.

The injection is obtained by dissolving the compound or a salt thereof in the present invention or the combination drug, and if desired, a benzoate and/or a salicylate, into water.

The above-mentioned salts of benzoic acid and salicylic acid include, for example, salts of alkali metals such as sodium, potassium etc., salts of alkaline earth metals such as calcium, magnesium etc., ammonium salts, meglumine salts, organic acid salts such as tromethamol, etc.

The concentration of the compound or a salt thereof in the present invention or the combination drug is from 0.5 w/v % to 50 w/v %, preferably from about 3 w/v % to about 20 w/v %. The concentration of a salt of benzoic acid or/and a salt of salicylic acid is from 0.5 w/v % to 50 w/v %, preferably from 3 w/v % to 20 w/v %.

Conventional additives to be used in an injection may be appropriately added in a preparation of the present invention. Examples of the additives include a stabilizer (e.g., ascorbic acid, sodium pyrosulfite, etc.), a surfactant (e.g., Polysorbate 80, macrogol etc.), a solubilizer (e.g., glycerin, ethanol etc.), a buffer (e.g., phosphoric acid and alkali metal salt thereof, citric acid and alkali metal salt thereof, etc.), an isotonizing agent (e.g., sodium chloride, potassium chloride, etc.), a dispersing agent (e.g., hydroxypropylmethyl cellulose, dextrin), a pH regulator (e.g., hydrochloric acid, sodium hydroxide etc.), an antiseptic (e.g., ethyl paraoxybenzoate, benzoic acid etc.), a dissolving agent (e.g., conc. glycerin, meglumine etc.), a solubilizing agent (e.g., propylene glycol, sucrose etc.), a soothing agent (e.g., glucose, benzyl alcohol etc.), etc. These additives are blended in a usual proportion generally employed in an injection.

It is advantageous that the pH of the injection is controlled from pH 2 to 12, preferably from 2.5 to 8.0 by addition of a pH regulator.

An injection is obtained by dissolving the compound or a salt thereof in the present invention or the combination drug and if desired, a salt of benzoic acid and/or a salt of salicylid acid, and if necessary, the above-mentioned additives into water. These may be dissolved in any order, and can be appropriately dissolved in the same manner as in a conventional method of producing an injection.

An aqueous solution for injection may be advantageously heated, alternatively, for example, filter sterilization, high pressure heat sterilization, etc. can be conducted in the same manner as those for a usual injection, to provide an injection.

It may be advantageous that an aqueous solution for injection is subjected to high pressure heat sterilization at 100° C. to 121° C. for 5 minutes to 30 minutes.

Further, a preparation endowed with the antibacterial property of a solution may also be produced so that it can be used as a preparation which is divided and administered multiple-times.

### [2] Sustained Release Preparation or Rapid Release Preparation, and Preparation Thereof

Preferred is a sustained release preparation which is obtained, by coating a core containing the compound of the present invention or the combination drug with a film forming agent such as a water-insoluble substance, swellable polymer, etc., if desired. For example, a sustained release preparation for oral once-a-day administration is preferable.

The water insoluble substance used in a film forming agent includes, for example, a cellulose ether such as ethyl cellulose, butyl cellulose, etc.; a cellulose ester such as cellulose acetate, cellulose propionate, etc.; a polyvinyl ester such as polyvinyl acetate, polyvinyl butyrate, etc.; an acrylic acid polymer such as acrylic acid/methacrylic acid copolymer, methylmethacrylate copolymer, ethoxyethyl methacrylate/cinnamoethylmethacrylate/aminoalkyl methacrylate copolymer, polyacrylic acid, polymethacrylic acid, methacrylic acid alkyl amide copolymer, poly(methyl methacrylate), polymethacrylate, polymethacryl amide, amino alkyl methacrylate copolymer, poly(methacrylic acid anhydride), glycidyl methacrylate copolymer, specially an Eudragit (manufactured by Rohm Pharma) such as Eudragit RS-100, RL-100, RS-30D, RL-30D, RL-PO, RS-PO (copolymer of ethyl acylate/methyl methacrylate/trimethyl chloride methacrylate/ammonium ethyl), Eudragit NE-30D (copolymer of methyl methacrylate/ethyl acrylate), etc., a hydrogenated oil such as hardened caster oil (e.g., Lovely wax (Freund Corporation), etc.), etc.; a wax such as carnauba wax, fatty acid glycerin ester, paraffin, etc.; polyglycerin fatty acid ester, etc.

The swellable polymer is preferably a polymer having acidic dissociating group and pH-dependent swelling property, and a polymer having acidic dissociating group which swells little in an acidic area such as stomach and swells greatly in a neutral area such as the small intestine or the large intestine.

The polymer having acidic dissociating group and pH-dependent swelling property includes, for example, crosslinkable polyacrylic polymer such as Carbomer 934P, 940, 941, 974P, 980, 1342 etc., polycarbophil, calcium polycarbophil (all are manufactured by BF Goodrich.), Hibiswako 103, 104, 105, 304 (all are manufactured by Wako Pure Chemical Industries, Ltd.), etc.

The film forming agent used in a sustained release preparation may further contain a hydrophilic substance.

The hydrophilic substance includes, for example, a polysaccharide optionally having sulfuric acid group such as pullulans, dextrin, arginic acid alkali metal salt, etc.; a polysaccharide having a hydroxyalkyl group or a carboxyalkyl group such as hydroxypropyl cellulose, hydroxypropyl methyl cellulose, sodium carboxymethyl cellulose, etc.; methyl cellulose; polyvinyl pyrrolidone; polyvinyl alcohol; polyethylene glycol; etc.

The content of water-insoluble substance in the film forming agent of sustained release preparation is about 30% (w/w) to about 90% (w/w), preferably about 35% (w/w) to about 80% (w/w), and more preferably about 40% (w/w) to about 75% (w/w). The content of swellable polymer is about 3% (w/w) to about 30% (w/w), preferably about 3% (w/w) to about 15% (w/w). The film forming agent may further contain a hydrophilic substance, in this case, the content of the hydrophilic substance in the film forming agent is about 50% (w/w) or less, preferably about 5% (w/w) to about 40% (w/w), and more preferably about 5% (w/w) to about 35% (w/w). This % (w/w) indicates % by weight based on a film forming agent composition which is obtained by removing a solvent (e.g., water, lower alcohols such as methanol, ethanol etc.) from a film forming agent liquid.

The sustained release preparation is manufactured by preparing a core containing drug, then, coating the resultant core with a film forming agent liquid prepared by heating and dissolving a water-insoluble substance, swellable polymer, etc. or by dissolving or dispersing it in a solvent as exemplified below.

### I. Preparation of Core Containing a Drug

The form of a core containing a drug to be coated with a film forming agent (hereinafter, sometimes simply referred to as the core) is not particularly limited, and preferably, the core is formed into particles such as granules or fine particles.

When the core is composed of granules or fine particles, the average particle size thereof is preferably from about 150 to about 2,000 μm, further preferably, from about 500 μm to about 1,400 μm.

The core can be prepared by a conventional method in which a suitable excipient, binding agent, disintegrating agent, lubricant, stabilizer, etc. are mixed with a drug, and the mixture is subjected to a wet-extrusion granulating method or a fluidized bed granulating method, etc.

The content of drugs in a core is from about 0.5% (w/w) to about 95% (w/w), preferably from about 5.0% (w/w) to about 80% (w/w), further preferably from about 30% (w/w) to about 70% (w/w).

The excipient contained in the core includes, for example, saccharides such as sucrose, lactose, mannitol, glucose etc., starch, crystalline cellulose, calcium phosphate, corn starch etc. Among them, crystalline cellulose, corn starch are preferable.

The binding agents include, for example, polyvinyl alcohol, hydroxypropyl cellulose, polyethylene glycol, polyvinyl pyrrolidone, Pluronic F68, gum arabic, gelatin, starch, etc. The disintegrating agents include, for example, carboxymethyl cellulose calcium (ECG505), croscarmellose sodium (Ac-Di-Sol), crosslinkable polyvinyl pyrrolidone (crospovidone), low-substituted hydroxypropyl cellulose (L-HPC), etc. Among these, hydroxypropyl cellulose, polyvinyl pyrrolidone and low-substituted hydroxypropyl cellulose are preferable. The lubricants or the aggregation inhibitor includes, for example, talc, magnesium stearate and an inorganic salt thereof. The lubricant includes a polyethylene glycol, etc. The stabilizers include an acid such as tartaric acid, citric acid, succinic acid, fumaric acid, maleic acid, etc.

In addition to the above-mentioned production method, the core can also be prepared by, for example, a rolling granulation method in which a drug or a mixture of the drug with an excipient, lubricant, etc. is added portionwise onto an inert carrier particle which is the core of the core while spraying a binder dissolved in a suitable solvent such as water, lower alcohol (e.g., methanol, ethanol, etc.) etc., a pan coating method, a fluidized bed coating method or a melt granulating method. The inert carrier particle includes, for example, those made of sucrose, lactose, starch, crystalline cellulose or waxes, and the average particle size thereof is preferably from about 100 μm to about 1,500 μm.

For the purpose of separating the drug contained in the core from the film forming agent, the surface of the core may be coated with a protective agent. The protective agent includes, for example, the above-mentioned hydrophilic substances, water-insoluble substances etc. The protective agent includes, preferably polyethylene glycol, and polysaccharides having hydroxyalkyl or carboxyalkyl, more preferably, hydroxypropylmethyl cellulose and hydroxypropyl cellulose. The protective agent may contain a stabilizer such as acids such as tartaric acid, citric acid, succinic acid, fumaric acid, maleic acid etc., and a lubricant such as talc etc. When the protective agent is used, the coating amount is from about 1% (w/w) to about 15% (w/w), preferably from about 1% (w/w) to about 10% (w/w), further preferably from about 2% (w/w) to about 8% (w/w), based on the core.

The coating of the protective agent can be carried out by a usual coating method, and specifically, the coating can be carried out by spraying the protective agent onto a core by a fluidized bed coating method, pan coating method etc.

II. Coating of Core with a Film Forming Agent

A core obtained in the above-mentioned step I is coated with a film forming agent liquid obtained by heating and dissolving the above-mentioned water-insoluble substance and pH-dependent swellable polymer, and a hydrophilic substance, or by dissolving or dispersing them in a solvent, to give a sustained release preparation.

The method for coating a core with a film forming agent liquid includes, for example, a spray coating method etc.

The composition ratio of a water-insoluble substance, swellable polymer and hydrophilic substance in a film forming agent liquid is appropriately selected so that the contents of these components in a coated film are the above-mentioned contents, respectively.

The coating amount of a film forming agent is from about 1% (w/w) to about 90% (w/w), preferably from about 5% (w/w) to about 50% (w/w), further preferably from about 5% (w/w) to 35% (w/w), based on a core (exclusive of the coating amount of the protective agent).

The solvent in the film forming agent liquid includes water or an organic solvent, alone or in admixture thereof. In the case of use in admixture, the mixing ratio of water to an organic solvent (water/organic solvent: weight ratio) can be varied in the range from 1 to 100%, and preferably from 1% to about 30%. The organic solvent is not particularly limited as long as it dissolves a water-insoluble substance, and for example, it includes lower alcohols such as methyl alcohol, ethyl alcohol, isopropyl alcohol, n-butyl alcohol, etc., lower alkanones such as acetone, etc., acetonitrile, chloroform, methylene chloride, etc. Among them, lower alcohols are preferable, and ethyl alcohol and isopropyl alcohol are particularly preferable. Water, and a mixture of water with an organic solvent are preferably used as a solvent for a film forming agent. In this case, if necessary, an acid such as tartaric acid, citric acid, succinic acid, fumaric acid, maleic acid, etc. may also be added into a film forming agent liquid for stabilizing the film forming agent liquid.

An operation of coating by spray coating can be conducted by a usual coating method, and specifically, it can be conducted by spray-coating a film forming agent liquid onto a core, for example, by a fluidized bed coating method, pan coating method etc. In this case, if necessary, talc, titanium oxide, magnesium stearate, calcium stearate, light anhydrous silicic acid etc. may also be added as a lubricant, and glycerin fatty acid ester, hydrogenated castor oil, triethyl citrate, cetyl alcohol, stearyl alcohol etc. may also be added as a plasticizer.

After coating with a film forming agent, if necessary, an antistatic agent such as talc etc. may be mixed.

The rapid release preparation may be liquid (solution, suspension, emulsion etc.) or solid (particle, pill, tablet etc.). It may be oral agents or parenteral agents such as an injection, etc., and preferably, oral agents.

The rapid release preparation, usually, may contain, in addition to an active component drug, also carriers, additives and excipients conventionally used in the field of formulation (hereinafter, sometimes abbreviated as the excipient). The preparation excipient used is not particularly limited as long as it is an excipient ordinarily used as a preparation excipient. For example, the excipient for an oral solid preparation includes lactose, starch, corn starch, crystalline cellulose (Avicel PH101, manufactured by Asahi Kasei Corporation, etc.), powder sugar, granulated sugar, mannitol, light anhydrous silicic acid, magnesium carbonate, calcium carbonate, L-cysteine, etc., and preferably, corn starch and mannitol, etc. These excipients can be used alone or in combination of two or more. The content of the excipient is, for example, from about 4.5 w/w % to about 99.4 w/w %, preferably from about 20 w/w % to about 98.5 w/w %, further preferably from about 30 w/w % to about 97 w/w %, based on the total amount of the rapid release preparation.

The content of a drug in the rapid release preparation can be appropriately selected in the range from about 0.5% to about 95%, preferably from about 1% to about 60% based on the total amount of the rapid release preparation.

When the rapid release preparation is an oral solid preparation, it usually contains a disintegrating agent in addition to the above-mentioned components. The disintegrating agent includes, for example, carboxymethyl cellulose calcium (ECG-505, manufactured by GOTOKU CHEMICAL COMPANY LTD.), croscarmellose sodium (e.g., acjizol, manufactured by Asahi Kasei Corporation), crospovidone (e.g., colidone CL, manufactured by BASF), low-substituted hydroxypropyl cellulose (manufactured by Shin-Etsu Chemical Co., Ltd.), carboxymethylstarch (manufactured by Matsutani Chemical Industry Co., Ltd.), carboxymethylstarch sodium (Exprotab, manufactured by Kimura Sangyo), partially α-starch (PCS, manufactured by Asahi Kasei Corporation), etc., and for example, includes those which disintegrate a granule by absorbing water in contact with water, causing swelling, or making a channel between an effective ingredient constituting the core and an excipient. These disintegrating agents can be used alone or in combinations of two or more. The amount of the disintegrating agent used is appropriately selected depending on the kind and blending amount of a drug used, formulation design for release property, etc., and for example, from about 0.05 w/w % to about 30 w/w %, preferably from about 0.5 w/w % to about 15 w/w %, based on the total amount of the rapid release preparation.

When the rapid release preparation is an oral solid preparation, it may further contain if desired, additives conventional in solid preparations in addition to the above-mentioned composition. Such an additive includes, for example, a binder (e.g., sucrose, gelatin, arabic gum powder, methyl cellulose, hydroxypropyl cellulose, hydroxypropylmethyl cellulose, carboxylmethyl cellulose, polyvinylpyrrolidone, pullulans, dextrin, etc.), a lubricant (e.g., polyethylene glycol, magnesium stearate, talc, light anhydrous silicic acid (e.g., aerosil (Nippon Aerosil)), a surfactant (e.g., anionic surfactants such as sodium alkylsulfate, etc., nonionic surfactants such as polyoxyethylene fatty acid ester and polyoxyethylene sorbitan fatty acid ester, polyoxyethylene castor oil derivatives, etc.), a colorant (e.g., tar coloring matter, caramel, iron oxide red, titanium oxide, riboflavins), if necessary, a corrigent (e.g., sweetening agent, flavor, etc.), an adsorbent, an antiseptic, a wetting agent, an antistatic agent, etc. Further, a stabilizer such as an organic acid such as tartaric acid, citric acid, succinic acid, fumaric acid, etc. may also be added.

The above-mentioned binder includes preferably hydroxypropyl cellulose, polyethylene glycol and polyvinylpyrrolidone, etc.

The rapid release preparation can be prepared by mixing the above-mentioned components, and if necessary, further kneading the mixture, and molding it based on a usual technology of producing preparations. The above-mentioned mixing is conducted by generally used methods, for example, mixing, kneading, etc. Specifically, when a rapid release preparation is formed, for example, into a particle, it can be prepared, according to the same means as in the above-mentioned method for preparing a core of a sustained release preparation, by mixing the components using a vertical granulator, universal kneader (manufactured by Hata Iron Works Co., Ltd.), fluidized bed granulator FD-5S (manufactured by Powrex Corporation), etc., then, subjecting the mixture to a wet extrusion granulation method, fluidized bed granulation method, etc.

Thus obtained rapid release preparation and sustained release preparation may be themselves made into products or made into products appropriately together with preparation excipients etc., separately, by an ordinary method, then, may be administered simultaneously or may be administered in combination at any administration interval, or they may be themselves made into one oral administration preparation (e.g., granule, fine particle, tablet, capsule etc.) or made into one oral administration preparation together with preparation excipients etc. It may also be permissible that they are made into granules or fine particles, and filled in the same capsule to be used as a preparation for oral administration.

### [3] Sublingual, Buccal or Intraoral Quick Disintegrating Agent and Preparation Thereof

Sublingual, buccal or intraoral quick disintegrating agents may be a solid preparation such as tablet etc., or may be an oral mucosa membrane patch (film).

The sublingual, buccal or intraoral quick disintegrating agent is preferably a preparation containing the androgen receptor antagonists of the present invention or the combination drug and an excipient. It may contain also auxiliary agents such as a lubricant, isotonizing agent, hydrophilic carrier, water-dispersible polymer, stabilizer etc. Further, for easy absorption and increased bioavailability, β-cyclodextrin or β-cyclodextrin derivatives (e.g., hydroxypropyl-β-cyclodextrin etc.), etc. may also be contained.

The above-mentioned excipient includes lactose, sucrose, D-mannitol, starch, crystalline cellulose, light anhydrous silicic acid, etc. The lubricant includes magnesium stearate, calcium stearate, talc, colloidal silica, etc., and particularly preferably, magnesium stearate and colloidal silica. The isotonizing agent includes sodium chloride, glucose, fructose, mannitol, sorbitol, lactose, saccharose, glycerin, urea, etc., and particularly preferably, mannitol. The hydrophilic carrier includes swellable hydrophilic carriers such as crystalline cellulose, ethyl cellulose, crosslinkable polyvinylpyrrolidone, light anhydrous silicic acid, silicic acid, dicalcium phosphate, calcium carbonate etc., and particularly preferably, crystalline cellulose (e.g., microcrystalline cellulose, etc.). The water-dispersible polymer includes gums (e.g., gum tragacanth, acacia gum, guar gum), alginates (e.g., sodium alginate), cellulose derivatives (e.g., methyl cellulose, carboxymethyl cellulose, hydroxymethyl cellulose, hydroxypropyl cellulose, hydroxypropylmethyl cellulose), gelatin, aqueous starch, polyacrylic acids (e.g., Carbomer), polymethacrylic acid, polyvinyl alcohol, polyethylene glycol, polyvinylpyrrolidone, polycarbophil, ascorbate, palmitates, etc., and preferably, hydroxypropylmethyl cellulose, polyacrylic acid, alginate, gelatin, carboxymethyl cellulose, polyvinylpyrrolidone, polyethylene glycol, etc., particularly preferably, hydroxypropylmethyl cellulose. The stabilizer includes cysteine, thiosorbitol, tartaric acid, citric acid, sodium carbonate, ascorbic acid, glycine, sodium sulfite, etc., and particularly preferably, citric acid and ascorbic acid.

The sublingual, buccal or intraoral quick disintegrating agent can be manufactured by mixing the androgen receptor antagonists of the present invention or the combination drug and an excipient by a per se known method. Further, if desired, auxiliary agents such as a lubricant, isotonizing agent, hydrophilic carrier, water-dispersible polymer, stabilizer, colorant, sweetening agent, antiseptic etc. may be mixed. The sublingual, buccal or intraoral quick disintegrating agent is obtained by mixing the above-mentioned components simultaneously or at a time interval, then subjecting the mixture to tablet-making molding under pressure. For obtaining suitable hardness, it may also be permissible that the materials are moistened by using a solvent such as water, alcohol etc. if desired before and after the tablet making process, and after the molding, the materials are dried, to obtain a product.

In the case of molding into a mucosa membrane patch (film), the androgen receptor antagonists of the present invention or the combination drug and the above-mentioned water-dispersible polymer (preferably, hydroxypropyl cellulose, hydroxypropylmethyl cellulose), excipient etc. are dissolved in a solvent such as water etc., and the resulted solution is cast to give a film. Further, additives such as a plasticizer, a stabilizer, an antioxidant, an antiseptic, a colorant, a buffer, a sweetening agent etc. may also be added. For imparting suitable elasticity to the film, glycols such as polyethylene glycol, propylene glycol, etc. may be contained, or for enhancing adhesion of the film to an intraoral mucosa membrane lining, a bio-adhesive polymer (e.g., polycarbophil, carbopol) may also be contained. In the casting, a solution is poured on the non-adhesive surface, spread to uniform thickness (preferably, about 10 micron to about 1,000 micron) by an application tool such as a doctor blade etc., then, the solution is dried to form a film. It may be advantageous that thus formed film is dried at room temperature or under heat, and cut into given area.

The intraoral quick disintegrating preparation is preferably solid quick diffuse preparation composed of a network body comprising the androgen receptor antagonists of the present invention or the combination drug, and a water-soluble or water-diffusible carrier which is inert to the androgen receptor antagonists of the present invention or the combination drug. This network body is obtained by sublimating a solvent from the composition constituted of a solution prepared by dissolving the androgen receptor antagonists of the present invention or the combination drug in a suitable solvent.

The composition of an intraoral quick disintegrating agent preferably contains a matrix forming agent and a secondary component in addition to the androgen receptor antagonists of the present invention or the combination drug.

The matrix forming agent includes animal proteins or vegetable proteins such as gelatins, dextrins, soybean, wheat and psyllium seed protein etc.; rubber substances such as arabic gum, guar gum, agar, xanthane, etc.; polysaccharides; alginic acids; carboxymethyl celluloses; carrageenans; dextrans; pectins; synthetic polymers such as polyvinylpyrrolidone, etc.; substances derived from a gelatin-arabic gum complex, etc. Further, it includes saccharides such as mannitol, dextrose, lactose, galactose, trehalose, etc.; cyclic saccharides such as cyclodextrin etc.; inorganic salts such as sodium phosphate, sodium chloride and aluminum silicate, etc.; amino acids having 2 to 12 carbon atoms such as glycine, L-alanine, L-aspartic acid, L-glutamic acid, L-hydroxyproline, L-isoleucine, L-leucine, L-phenylalanine, etc.

One or more of the matrix forming agent(s) can be introduced in a solution or suspension before solidification. Such matrix forming agent may be present in addition to a surfactant, or may be present with the surfactant excluded. The matrix forming agents may help to keep the androgen receptor antagonists of the present invention or the combination drug diffused in the solution or suspension, in addition to formation of the matrix.

The composition may contain secondary components such as a preservative, an antioxidant, a surfactant, a thickening agent, a colorant, a pH controlling agent, a flavoring agent, a sweetening agent, a food taste masking agent, etc. The suitable colorant includes red, black and yellow iron oxides, and FD & C dyes such as FD & C Blue 2, FD & C Red 40, etc. manufactured by Elis and Eberald. Examples of the suitable flavoring agent include mint, raspberry, licorice, orange, lemon, grape fruit, caramel, vanilla, cherry, grape flavor and combinations thereof. Examples of the suitable pH controlling agent include citric acid, tartaric acid, phosphoric acid, hydrochloric acid and maleic acid. Examples of the suitable sweetening agent include aspartame, acesulfame K and thaumatine, etc. Examples of the suitable food taste masking agent include sodium bicarbonate, ion exchange resin, cyclodextrin-inclusion compounds, adsorbent substances and microcapsulated apomorphine.

The preparation contains the androgen receptor antagonists of the present invention or the combination drug in an amount usually from about 0.1% by weight to about 50% by weight, preferably from about 0.1% by weight to about 30% by weight, and is preferably a preparation (such as the above-mentioned sublingual agent, buccal etc.) which can dissolve 90% or more the androgen receptor antagonists of the present invention or the combination drug (into water) within the time range of about 1 minute to about 60 minutes, preferably of about 1 minute to 15 minutes, more preferably of about 2 minutes to about 5 minutes, and intraoral quick disintegrating preparations which are disintegrated within the range of 1 second to 60 seconds, preferably of 1 to 30 seconds, further preferably of 1 to 10 seconds after being placed in the oral cavity.

The content of the above-mentioned excipient in the whole preparation is from about 10% by weight to about 99% by weight, lo preferably from about 30% by weight to about 90% by weight. The content of β-cyclodextrin or β-cyclodextrin derivative in the whole preparation is from 0 to about 30% by weight. The content of the lubricant in the whole preparation is from about 0.01% by weight to about 10% by weight, preferably from about 1% by weight to about 5% by weight. The content of the isotonizing agent in the whole preparation is from about 0.1% by weight to about 90% by weight, preferably, from about 10% by weight to about 70% by weight. The content of the hydrophilic carrier in the whole preparation is from about 0.1% by weight to about 50% by weight, preferably, from about 10% by weight to about 30% by weight. The content of the water-dispersible polymer in the whole preparation is from about 0.1 to about 30% by weight, preferably, from about 10% by weight to about 25% by weight. The content of the stabilizer in the whole preparation is from about 0.1% by weight to about 10% by weight, preferably, from about 1% by weight to about 5% by weight. The above-mentioned preparation may further contain additives such as a colorant, a sweetening agent, an antiseptic, etc., if necessary.

The dose of a combination preparation of the present invention differs depending on the kind of the compound of the present invention, age, body weight, condition, drug form, administration method, administration period etc., and for example, for a prostate cancer patient (adult, body weight: about 60 kg), the combination preparation is administered intravenously, at a dose of about 0.01 to about 1,000 mg/kg/day, preferably about 0.01 to about 100 mg/kg/day, more preferably about 0.1 to about 100 mg/kg/day, particularly about 0.1 to about 50 mg/kg/day, especially about 1.5 to about 30 mg/kg/day, in terms of the androgen receptor antagonists of the present invention or the combination drug, respectively, once or several times a day in divided portions. Of course, since the dose as described above varies depending on various conditions, it may be sometimes sufficient to administer smaller amounts than the above-mentioned dosage, and further, it may be sometimes necessary to administer greater amounts than that.

The amount of the combination drug can be set at any value unless side effects are problematical. The daily dosage in terms of the combination drug differs depending on the severity of symptoms, age, sex, body weight, sensitivity difference of the subject, administration time and interval, property, prescription, and kind of the pharmaceutical preparation, kind of effective ingredient, etc., and not particularly limited; for example, in the case of oral administration, the dose of the drug is usually from about 0.001 mg to 2,000 mg, preferably from about 0.01 mg to 500 mg, further preferably from about 0.1 mg to 100 mg, per 1 kg body weight of a mammal, which is usually administered once to four times a day in divided portions.

In administration of the combination preparation of the present invention, the androgen receptor antagonists of the present invention may be administered after administration of the combination drug or the combination drug may be administered after administration of the androgen receptor antagonists of the present invention, though they may be administered simultaneously. When administered at a time interval, the interval differs depending on the effective ingredient to be administered, drug form and administration method. For example, when the combination drug is administered first, the androgen receptor antagonists of the present invention is administered within time range of from 1 minute to 3 days, preferably from 10 minutes to 1 day, more preferably from 15 minutes to 1 hour after administration of the combined drug. When the androgen receptor antagonists of the present invention is administered first, the combined drug is administered within time range of from 1 minute to 1 day, preferably from 10 minutes to 6 hours, more preferably from 15 minutes to 1 hour after administration of the androgen receptor antagonists of the present invention.

In a preferable administration method, for example, the combination drug formulated into an oral administration preparation is administered orally at a daily dose of about 0.001 mg/kg to 200 mg/kg, and 15 minutes later, the androgen receptor antagonists of the present invention formulated into an oral administration preparation is administered orally at a daily dose of about 0.005 mg/kg to 100 mg/kg.

### EXAMPLES

The present invention is explained in more detail in the following by referring to Reference Examples, Examples, Preparation Examples and Experimental Examples, which are not to be construed as limitative.

The “room temperature” in the following Reference Examples means a temperature of generally from about 10° C. to 35° C. Unless otherwise specified, “%” means weight % and the yield is in mol/mol %.

NMR spectra are shown by proton NMR with tetramethylsilane as the internal standard, using 200 MHz or 300 MHz spectrometer; δ values are expressed in ppm.

In the present specification, the melting point means one measured, for example, using a trace melting point measurement device (Yanaco, Buchi, B-545 and the like) or DSC (Differential Scanning Calorimetory) apparatus (SEIKO, EXSTAR6000 and the like) and the like.

In general, the melting points may vary depending on the measurement apparatuses, the measurement conditions and the like. The crystal in the present specification may show different values from the melting point described in the present specification as long as they are within general error range.

Other abbreviations used in the description mean the following.
s: singlet
brs: broad singlet
d: doublet
t: triplet
q: quartet
dd: double doublet
ddd: double double doublet
dt: double triplet
m: multiplet
CDCl₃: deuterated chloroform
DMSO-d₆: deuterated dimethyl sulfoxide
CD₃OD: deuterated methanol
¹H-NMR: proton nuclear magnetic resonance
THF: tetrahydrofuran
DMF: N,N-dimethylformamide

### Reference Example 1

### 1-(4-bromophenyl)-3,5-dimethyl-1H-pyrazole

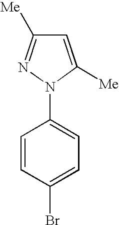

4-Bromophenylhydrazine hydrochloride (20.0 g) and acetylacetone (8.96 g) were heated under reflux in acetic acid (50 ml) for 3 hr. The solvent was evaporated under reduced pressure, and the obtained mixture was diluted with ethyl acetate and aqueous sodium hydrogen carbonate solution. The organic layer was separated, washed with water and saturated brine, dried over anhydrous magnesium sulfate and concentrated. The obtained residue was purified by column chromatography (ethyl acetate) to give the title compound (19.10 g) as a brown oil.

¹H-NMR (CDCl₃) δ: 2.28 (3H, s), 2.30 (3H, s), 6.00 (1H, s), 7.28-7.38 (2H, m), 7.51-7.62 (2H, m).

### Reference Example 2

1-(4-bromophenyl)-3,5-dimethyl-1H-pyrazole-4-carbaldehyde

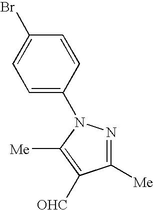

Phosphorus oxychloride (6.22 ml) was slowly added dropwise to DMF (5.17 ml) cooled to 5° C., and then the reaction mixture was stirred for 1 hr. A solution (5 ml) of 1-(4-bromophenyl)-3,5-dimethyl-1H-pyrazole (9.60 g) synthesized in Reference Example 1 in DMF was added dropwise to the reaction mixture, and the mixture was stirred at room temperature for 16 hr. The reaction mixture was poured into ice water, diluted with ethyl acetate, and the organic layer was separated. The organic layer was washed with aqueous sodium hydrogen carbonate solution and saturated brine, dried over anhydrous magnesium sulfate and concentrated. The obtained residue was purified by column chromatography (ethyl acetate-hexane) and recrystallized from ethyl acetate-hexane to give the title compound (8.60 g) as colorless crystals.

¹H-NMR (CDCl₃) δ: 2.42-2.63 (6H, m), 7.23-7.36 (2H, m), 7.58-7.73 (2H, m), 9.98-11.82 (1H, m).

### Reference Example 3

### 4-(4-formyl-3,5-dimethyl-1H-pyrazol-1-yl)benzonitrile

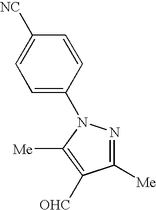

Under a nitrogen atmosphere, 1-(4-bromophenyl)-3,5-dimethyl-1H-pyrazole-4-carbaldehyde (4.60 g) synthesized in Reference Example 2, zinc cyanide (1.28 g) and tetrakis(triphenylphosphine)palladium (31 mg) were stirred in a solvent of DMF (5 ml) at 100° C. for 10 hr. The reaction mixture was diluted with aqueous ammonia and ethyl acetate, and the organic layer was separated, dried over anhydrous magnesium sulfate and concentrated. The obtained residue was recrystallized from ethyl acetate-hexane to give the title compound (2.55 g) as pale-yellow crystals.

¹H-NMR (CDCl₃) δ: 2.53 (3H, s), 2.64 (3H, s), 7.53-7.66 (2H, m), 7.77-7.88 (2H, m), 10.05 (1H, s).

### Reference Example 4

### [1-(4-bromo-3-chlorophenyl)-3,5-dimethyl-1H-pyrazol-4-yl](4-fluorophenyl)methanol

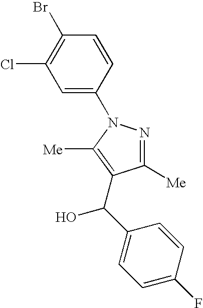

To a solution (10 ml) of 1-(4-bromo-3-chlorophenyl)-3,5-dimethyl-1H-pyrazole-4-carbaldehyde (450 mg) synthesized in Reference Example 13 in THF was added 1.50M 4-fluorophenylmagnesium bromide (5.74 ml), and the mixture was stirred at room temperature for 30 min. The reaction mixture was diluted with aqueous ammonium chloride solution and ethyl acetate, and the organic layer was separated, dried over anhydrous magnesium sulfate, and concentrated. The obtained residue was purified by column chromatography (ethyl acetate-hexane) and recrystallized from ethyl acetate-hexane to give the title compound (350 mg) as colorless crystals.

¹H-NMR (CDCl₃) δ: 2.03 (1H, d), 2.17 (3H, s), 2.24 (3H, s), 5.87-5.96 (1H, m), 6.99-7.10 (2H, m), 7.19 (1H, dd), 7.30-7.43 (2H, m), 7.57 (1H, d), 7.69 (1H, d).

### Reference Example 5

### [1-(4-bromo-3-chlorophenyl)-3,5-dimethyl-1H-pyrazol-4-yl](4-fluorophenyl)methanone

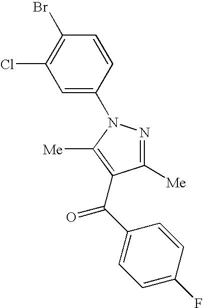

A solution (105 ml) of [1-(4-bromo-3-chlorophenyl)-3,5-dimethyl-1H-pyrazol-4-yl](4-fluorophenyl)methanol (280 mg) synthesized in Reference Example 4 and manganese dioxide (2.97 g) in DMF was stirred at room temperature for 8 hr. The reaction mixture was diluted with water and ethyl acetate, and filtered through celite. The filtrate was dried over anhydrous magnesium sulfate, and concentrated. The obtained residue was recrystallized from ethyl acetate-hexane to give the title compound (186 mg) as colorless crystals.

¹H-NMR (CDCl₃) δ: 2.21 (3H, s), 2.32 (3H, s), 7.12-7.26 (3H, m), 7.62 (1H, d), 7.71-7.86 (3H, m).

### Reference Example 6

### 1-[1-(4-bromo-3-chlorophenyl)-3,5-dimethyl-1H-pyrazol-4-yl]-1-(4-fluorophenyl)ethanol

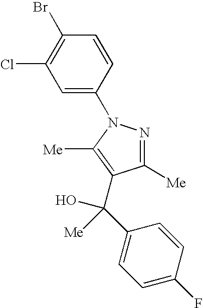

Using [1-(4-bromo-3-chlorophenyl)-3,5-dimethyl-1H-pyrazol-4-yl](4-fluorophenyl)methanone synthesized in Reference Example 5 and methylmagnesium bromide as starting materials and in the same manner as in Reference Example 4, the title compound was obtained.

¹H-NMR (CDCl₃) δ: 1.94 (3H, s), 2.02 (1H, s), 2.11-2.17 (6H, m), 6.96-7.08 (2H, m), 7.16 (1H, dd), 7.35-7.45 (2H, m), 7.54 (1H, d), 7.68 (1H, d).

### Reference Example 7

### 4-[4-(hydroxymethyl)-3,5-dimethyl-1H-pyrazol-1-yl]benzonitrile

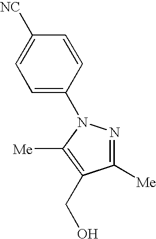

After cooling a solution (30 ml/10 ml) of 4-(4-formyl-3,5-dimethyl-1H-pyrazol-1-yl)benzonitrile (800 mg) synthesized in Reference Example 3 in THF and ethanol to 5° C., sodium borohydride (269 mg) was added and the mixture was stirred for 1 hr. The reaction mixture was diluted with aqueous sodium hydrogen carbonate solution and ethyl acetate, and the aqueous layer was extracted with ethyl acetate. The obtained organic layer was dried over anhydrous magnesium sulfate and concentrated. The obtained residue was purified by column chromatography (ethyl acetate-hexane) and recrystallized from ethyl acetate-hexane to give the title compound (590 mg) as colorless crystals.

¹H-NMR (CDCl₃) δ: 1.35 (1H, t), 2.35 (3H, s), 2.41 (3H, s), 4.57 (2H, d), 7.56-7.64 (2H, m), 7.70-7.81 (2H, m).

### Reference Example 8

### [1-(4-bromophenyl)-3,5-dimethyl-1H-pyrazol-4-yl](4-fluorophenyl)methanone

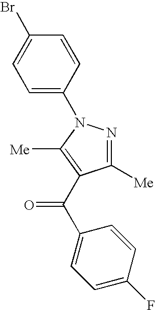

1-(4-Bromophenyl)-3,5-dimethyl-1H-pyrazole (1.00 g) obtained in Reference Example 1 and 4-fluorobenzoyl chloride (947 mg) were dissolved in dichloromethane (15 ml) and cooled to 5° C. Aluminum chloride (796 mg) was added and the mixture was stirred for 5 hr. The reaction mixture was added to ice-cooled water and diluted with ethyl acetate and 1 mol/L hydrochloric acid. The organic layer was separated, washed with aqueous sodium hydrogen carbonate solution, dried over anhydrous magnesium sulfate, and concentrated. The obtained residue was purified by column chromatography (ethyl acetate-hexane) and recrystallized from ethyl acetate-hexane to give the title compound (660 mg) as colorless crystals.

¹H-NMR (CDCl₃) δ: 2.21 (3H, s), 2.30 (3H, s), 7.12-7.22 (2H, m), 7.31-7.40 (2H, m), 7.60-7.68 (2H, m), 7.75-7.88 (2H, m).

### Reference Example 9

### 4-[4-(1-hydroxyethyl)-3,5-dimethyl-1H-pyrazol-1-yl]benzonitrile

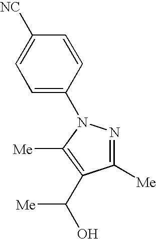

Using 4-(4-formyl-3,5-dimethyl-1H-pyrazol-1-yl)benzonitrile obtained in Reference Example 3 and methylmagnesium bromide as starting materials and in the same manner as in Reference Example 4, the title compound was obtained.

¹H-NMR (CDCl₃) δ: 1.57 (3H, d), 1.63 (1H, d), 2.37 (3H, s), 2.41 (3H, s), 4.92-5.05 (1H, m), 7.51-7.61 (2H, m), 7.70-7.79 (2H, m).

### Reference Example 10

### 4-(4-benzyl-3-methyl-5-oxo-4,5-dihydro-1H-pyrazol-1-yl)benzonitrile

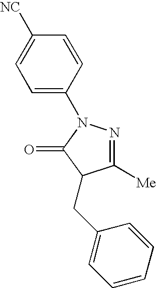

Using 4-benzyl-2-(4-bromophenyl)-5-methyl-2,4-dihydro-3H-pyrazol-3-one obtained in Reference Example 11 as a starting material and in the same manner as in Reference Example 3, the title compound was obtained.

¹H-NMR (CDCl₃) δ: 2.02-2.21 (3H, m), 3.17-3.38 (2H, m), 3.54-3.70 (1H, m), 7.12-7.33 (5H, m), 7.60-7.71 (2H, m), 7.84-8.07 (2H, m).

### Reference Example 11

### 4-benzyl-2-(4-bromophenyl)-5-methyl-2,4-dihydro-3H-pyrazol-3-one

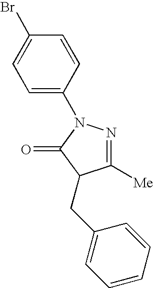

4-Bromophenylhydrazine hydrochloride (5.00 g) and ethyl 2-benzylacetoacetate (4.93 g) were heated under reflux in an acetic acid solvent (15 ml) for 3 hr. The reaction mixture was cooled to room temperature, and diluted with ethyl acetate and aqueous sodium hydrogen carbonate solution. The organic layer was separated, washed with water and saturated brine, dried over anhydrous magnesium sulfate, and concentrated. The obtained residue was recrystallized from methanol-THF-ethyl acetate to give the title compound (4.20 g) as colorless crystals.

¹H-NMR (CDCl₃) δ: 1.99-2.18 (3H, m), 3.17-3.35 (2H, m), 3.49-3.72 (1H, m), 7.14-7.33 (5H, m), 7.43-7.55 (2H, m), 7.58-7.77 (2H, m).

### Reference Example 12

### 1-(4-bromo-3-chlorophenyl)-3,5-dimethyl-1H-pyrazole

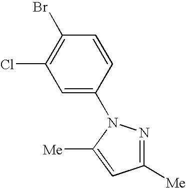

Using (4-bromo-3-chlorophenyl)hydrazine hydrochloride obtained in Reference Example 15 as a starting material and in the same manner as in Reference Example 1, the title compound was obtained.

¹H-NMR (CDCl₃) δ: 2.37 (3H, s), 2.53 (3H, s), 6.23 (1H, s), 7.36 (1H, dd), 7.60 (1H, d), 7.82 (1H, d).

### Reference Example 13

### 1-(4-bromo-3-chlorophenyl)-3,5-dimethyl-1H-pyrazole-4-carbaldehyde

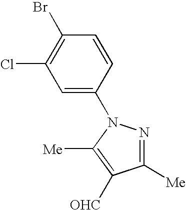

Using 1-(4-bromo-3-chlorophenyl)-3,5-dimethyl-1H-pyrazole obtained in Reference Example 12 as a starting material and in the same manner as in Reference Example 2, the title compound was obtained.

¹H-NMR (CDCl₃) δ: 2.52 (3H, s), 2.58 (3H, s), 7.22 (1H, dd), 7.59 (1H, d), 7.75 (1H, d), 10.02 (1H, s).

### Reference Example 14

### [1-(4-bromo-3-chlorophenyl)-3,5-dimethyl-1H-pyrazol-4-yl](phenyl)methanol

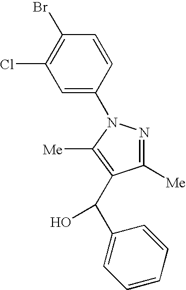

Using 1-(4-bromo-3-chlorophenyl)-3,5-dimethyl-1H-pyrazole-4-carbaldehyde obtained in Reference Example 13 and phenylmagnesium bromide as starting materials and in the same manner as in Reference Example 4, the title compound was obtained.

³H-NMR (CDCl₃) δ: 2.05 (1H, d), 2.19 (3H, s), 2.25 (3H, s), 5.94 (1H, d), 7.19 (1H, dd), 7.24-7.45 (5H, m), 7.56 (1H, d), 7.68 (1H, d).

### Reference Example 15

### (4-bromo-3-chlorophenyl)hydrazine hydrochloride

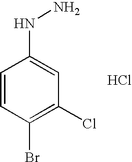

4-Bromo-3-chloroaniline (50 g) was suspended in acetic acid/methanol/concentrated hydrochloric acid/water (200 ml/200 ml/300 ml/300 ml) and cooled to 5° C. A solution of sodium nitrite (16.71 g) in water (100 ml) was added dropwise slowly, and the mixture was stirred for 1 hr. The reaction mixture was cooled to 5° C. and added to an excess amount of aqueous sodium dithionite solution. The mixture was stirred for 2 hr, and the precipitated solid was collected by filtration. The obtained solid was washed with water and diethyl ether, and dried to give the title compound (18.3 g) as pale-yellow crystals.

¹H-NMR (DMSO-d₆) δ: 6.87 (1H, dd), 7.22 (1H, d), 7.64 (1H, d), 8.65 (1H, brs), 10.27 (3H, brs).

### Reference Example 16

### methyl 4-[(3,5-dimethyl-1H-pyrazol-4-yl)oxy]benzoate

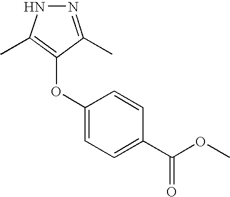

To a solution of methyl 4-hydroxybenzoate (5.00 g) in DMF (125 ml) were added potassium carbonate (9.10 g) and 3-chloropentane-2,4-dione (7.84 ml) at 0° C., and the mixture was heated to 80° C. and stirred for 30 min. The reaction mixture was allowed to cool, and an insoluble solid was removed by filtration. The filtrate was diluted with ethyl acetate, washed with saturated aqueous ammonium chloride solution and saturated brine, dried over anhydrous sodium sulfate, and concentrated. A solution of the obtained methyl 4-(1-acetyl-2-oxopropoxy)benzoate (11.8 g) and hydrazine monohydrate (1.94 ml) in acetic acid (40 ml) was stirred at room temperature for 1 hr, and concentrated. The residue was cooled to 0° C., poured into saturated aqueous sodium hydrogen carbonate, and the mixture was extracted with ethyl acetate. The ethyl acetate layer was washed with saturated aqueous sodium hydrogen carbonate and saturated brine, dried over anhydrous sodium sulfate, and concentrated. The residue was purified by column chromatography (hexane-ethyl acetate) to give the title compound (4.20 g) as white crystals.

¹H-NMR (CDCl₃) δ: 2.11 (6H, s), 3.89 (3H, s), 6.91 (2H, d), 7.98 (2H, d).

### Reference Example 17

### 2-chloro-4-(4-formyl-3,5-dimethyl-1H-pyrazol-1-yl)benzonitrile

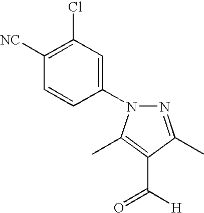

Phosphorus oxychloride (14.4 ml) was added dropwise to DMF (65 ml) cooled to 0° C., and the mixture was stirred for 15 min to give a Vilsmeier reagent. To the reagent was added a solution of 2-chloro-4-(3,5-dimethyl-1H-pyrazol-1-yl)benzonitrile (15.0 g) synthesized in Reference Example 29 in DMF (90 ml). The reaction mixture was heated to 80° C., stirred for 12 hr, ice-cooled, and water was added thereto. The insoluble solid was collected by filtration, and washed with methanol and petroleum ether to give the title compound (4.58 g) as a yellow solid. The filtrate was extracted with ethyl acetate, and the obtained ethyl acetate layer was washed with saturated aqueous sodium hydrogen carbonate and saturated brine, dried over anhydrous sodium sulfate, and concentrated. The residue was purified by column chromatography (hexane-ethyl acetate) to give the title compound (4.79 g) as a yellow solid.

¹H-NMR (CDCl₃) δ: 2.53 (3H, s), 2.67 (3H, s), 7.51 (1H, d), 7.73 (1H, s), 7.81 (1H, d), 10.05 (1H, s).

### Reference Example 18

### 2-chloro-4-(4-hydroxy-3,5-dimethyl-1H-pyrazol-1-yl)benzonitrile

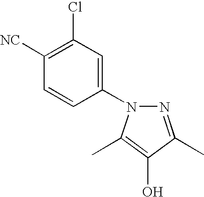

To a solution of 2-chloro-4-(4-formyl-3,5-dimethyl-1H-pyrazol-1-yl)benzonitrile (2.00 g) synthesized in Reference Example 17 in acetonitrile (42 ml)-ethyl acetate (26 ml) was added 3-chloroperbenzoic acid (3.99 g), and the mixture was stirred at room temperature for 16 hr. The reaction mixture was washed with aqueous sodium thiosulfate solution, saturated aqueous sodium hydrogen carbonate and saturated brine, dried over anhydrous sodium sulfate, and concentrated. The obtained 1-(3-chloro-4-cyanophenyl)-3,5-dimethyl-1H-pyrazol-4-yl formate as a yellow solid was dissolved in methanol (30 ml), triethylamine (3.22 ml) was added, and the mixture was stirred at room temperature for 1 hr. The reaction mixture was concentrated, and the residue was purified by column chromatography (hexane-ethyl acetate) to give the title compound (1.31 g) as a yellow solid.

¹H-NMR (DMSO-d₆) δ: 2.14 (3H, s), 2.34 (3H, s), 7.69 (1H, d), 7.87 (1H, s), 8.02 (1H, d), 8.35 (1H, brs).

### Reference Example 19

### [1-(3-chloro-4-cyanophenyl)-3,5-dimethyl-1H-pyrazol-4-yl](oxo)acetyl chloride

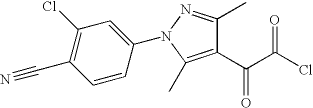

2-Chloro-4-(3,5-dimethyl-1H-pyrazol-1-yl)benzonitrile (1.29 g) synthesized in Reference Example 29 was suspended in oxalyl chloride (2.50 ml), and the mixture was stirred with heating at 80° C. for 3 hr. Excess oxalyl chloride was evaporated under reduced pressure at 80-100° C. and the obtained residue was washed with hexane to give the title compound (1.76 g).

MS (dissolved in MeOH, ESI+, m/e) 318 (M+1)

¹H-NMR (CDCl₃) δ: 2.46 (3H, s), 2.62 (3H, s), 7.49 (1H, dd), 7.71 (1H, d), 7.85 (1H, d).

### Reference Example 20

### 1-(3-chloro-4-cyanophenyl)-3,5-dimethyl-1H-pyrazole-4-carbonyl chloride

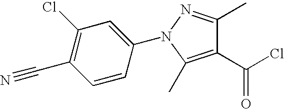

2-Chloro-4-(3,5-dimethyl-1H-pyrazol-1-yl)benzonitrile (6.05 g) synthesized in Reference Example 29 was suspended in oxalyl chloride (56.0 ml), and the mixture was stirred with heating at 80° C. for 23 hr. Excess oxalyl chloride was evaporated under reduced pressure at 140° C. and the obtained residue was washed with hexane to give the title compound (7.58 g).

MS (dissolved in MeOH, ESI+, m/e) 290 (M+1)

### Reference Example 21

### 2-chloro-4-[4-(hydroxymethyl)-3,5-dimethyl-1H-pyrazol-1-yl]benzonitrile

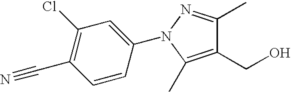

1-(3-Chloro-4-cyanophenyl)-3,5-dimethyl-1H-pyrazole-4-carbonyl chloride (2.36 g) synthesized in Reference Example 20 was dissolved in THF (40 ml), aqueous sodium borohydride (1.59 g) solution (4.20 ml) was added under ice-cooling and the mixture was stirred at room temperature for 3 hr. 2 mol/l Hydrochloric acid (24.0 ml) was added at room temperature and the mixture was stirred for 30 min, and extracted with ethyl acetate. The organic layer was washed with 5% aqueous sodium bicarbonate and saturated brine, dried over anhydrous magnesium sulfate, filtered through silica gel column (1.0 g) and concentrated. The obtained residue was washed with diisopropyl ether to give the title compound (2.00 g) as white crystals.

MS (ESI+, m/e) 262 (M+1)

¹H-NMR (DMSO-d₆) δ: 2.22 (3H, s), 2.40 (3H, s), 4.32 (1H, d), 4.75 (1H, t), 7.72 (1H, dd), 7.91 (1H, d), 8.08 (1H, d).

### Reference Example 22

### 1-(3-chloro-4-cyanophenyl)-3,5-dimethyl-1H-pyrazole-4-carboxylic acid

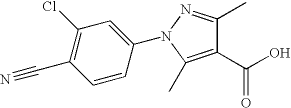

Ice water was added to 1-(3-chloro-4-cyanophenyl)-3,5-dimethyl-1H-pyrazole-4-carbonyl chloride (0.414 g) synthesized in Reference Example 20 and the mixture was stirred at room temperature for 5 hr. The precipitate was collected by filtration, washed with water and diisopropyl ether and dried. The obtained solid was recrystallized from water-ethanol to give the title compound (24.1 mg).

MS (ESI+, m/e) 276 (M+1)

¹H-NMR (DMSO-d₆) δ: 2.38 (3H, s), 2.58 (3H, s), 7.76 (1H, dd), 8.00 (1H, d), 8.15 (1H, d), 12.59 (1H, brs).

### Reference Example 23

### 4-(4-amino-3,5-dimethyl-1H-pyrazol-1-yl)-2-chlorobenzonitrile

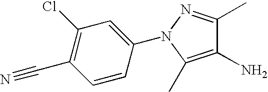

1-(3-Chloro-4-cyanophenyl)-3,5-dimethyl-1H-pyrazole-4-carboxylic acid (0.166 g) synthesized in Reference Example 22 and triethylamine (0.101 g) were dissolved in toluene (3.34 ml), and diphenylphosphoryl azide (0.136 g) was added dropwise under ice-cooling. The reaction mixture was stirred at 0° C. for 2.5 hr, water (3.5 ml) was added and the mixture was extracted with diethyl ether. The organic layer was washed with saturated brine, dried over anhydrous magnesium sulfate and concentrated. The obtained residue was dissolved again in toluene (6.67 ml), and the mixture was heated under reflux for 3 hr. 2 mol/l Hydrochloric acid (1.8 ml) was added and the mixture was heated under reflux for 1 hr, cooled to room temperature and diluted with 2.33 mol/l hydrochloric acid (15.0 ml). The partitioned aqueous layer was neutralized with potassium carbonate, and extracted with ethyl acetate. The organic layer was dried over anhydrous magnesium sulfate and concentrated. The residue was suspended in diisopropyl ether and collected by filtration to give the title compound (54.7 mg).

MS (ESI+, m/e) 247 (M+1)

¹H-NMR (DMSO-d₆) δ: 2.11 (3H, s), 2.32 (3H, s), 3.95 (2H, brs), 7.65 (1H, dd), 7.83 (1H, d), 7.98 (1H, d).

### Reference Example 24

### 4-(3,5-dimethyl-1H-pyrazol-1-yl)benzonitrile

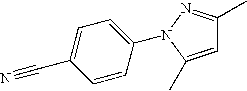

3,5-Dimethylpyrazole (16.0 g) was dissolved in DMF (196 ml), sodium hydride (5.27 g) was added at 0° C., and the mixture was stirred at 0° C. for 10 min. Sodium hydride (3.52 g) was further added, and the mixture was stirred at 0° C. for 10 min and at room temperature for 30 min. 4-Fluorobenzonitrile (20.2 g) was added, and the mixture was stirred at room temperature for 8 hr. The mixture was poured into ice water (300 ml), and the mixture was extracted with ethyl acetate. The organic layer was washed with water and saturated brine, dried over anhydrous magnesium sulfate and concentrated. Recrystallization from diisopropyl ether gave the title compound (27.6 g).

MS (ESI+, m/e) 198 (M+1)

¹H-NMR (DMSO-d₆) δ: 2.19 (3H, s), 2.39 (3H, s), 6.16 (1H, s), 7.75 (2H, d), 7.96 (2H, d).

### Reference Example 25

### 1-(4-cyanophenyl)-3,5-dimethyl-1H-pyrazole-4-carbonyl chloride

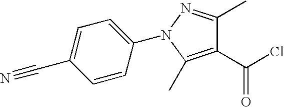

Using 4-(3,5-dimethyl-1H-pyrazol-1-yl)benzonitrile (10.0 g) synthesized in Reference Example 24 as a starting material and in the same manner as in Reference Example 20, the title compound (11.7 g) was obtained.

MS (dissolved in MeOH, ESI+, m/e) 256 (M+1)

### Reference Example 26

### 4-[4-(hydroxymethyl)-3,5-dimethyl-1H-pyrazol-1-yl]benzonitrile

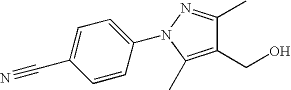

Using 1-(4-cyanophenyl)-3,5-dimethyl-1H-pyrazole-4-carbonyl chloride (4.11 g) synthesized in Reference Example 25 as a starting material and in the same manner as in Reference Example 21, the title compound (2.85 g) was obtained.

MS (ESI+, m/e) 228 (M+1)

¹H-NMR (DMSO-d₆) δ: 2.22 (3H, s), 2.36 (3H, s), 4.32 (2H, s), 4.71 (1H, brs), 7.73 (2H, d), 7.96 (2H, d).

### Reference Example 27

### 4-(4-fluorophenoxy)-3,5-dimethyl-1H-pyrazole

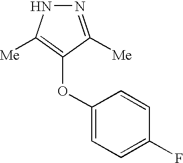

A mixture of 4-fluorophenol (1.00 g), 3-chloropentane-2,4-dione (1.17 mL), cesium carbonate (3.20 g) and acetone (20 mL) was heated under reflux with stirring for 5 hr. After cooling the reaction mixture, white precipitate was filtered off, and the filtrate was concentrated under reduced pressure. The residue was purified by column chromatography (hexane-ethyl acetate) to give a pale-yellow oil (1.30 g). The oil was dissolved in acetic acid (12 mL), hydrazine monohydrate (0.333 mL) was added, and the mixture was stirred at room temperature for 1 hr. The reaction mixture was concentrated under reduced pressure and neutralized with saturated aqueous sodium hydrogen carbonate solution. The mixture was extracted twice with ethyl acetate. The organic layers were combined, washed with saturated brine, dried over anhydrous magnesium sulfate and concentrated. The residue was purified by column chromatography (hexane-ethyl acetate) to give the title compound (0.499 g) as a yellow solid.

¹H-NMR (CDCl₃) δ: 2.11 (6H, s), 6.78-6.87 (2H, m), 6.91-7.00 (2H, m).

### Reference Example 28

### N-[4-(aminocarbonyl)benzyl]-1-[4-(aminocarbonyl)-3-chlorophenyl]-3,5-dimethyl-1H-pyrazole-4-carboxamide

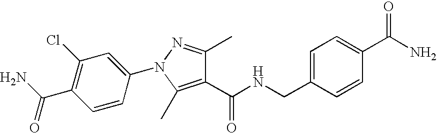

Methyl 4-[({[1-(3-chloro-4-cyanophenyl)-3,5-dimethyl-1H-pyrazol-4-yl]carbonyl}amino)methyl]benzoate (0.190 g) synthesized in Example 32 was dissolved in THF-methanol (1:1, 10.7 ml), 1 mol/l aqueous sodium hydroxide solution (28.0 ml) was added, and the mixture was stirred at room temperature for 1 hr. Methanol (3.5 ml) was further added, and the mixture was stirred at room temperature for 15 hr. The reaction mixture was concentrated and neutralized with hydrochloric acid. The obtained precipitate was collected by filtration, washed with water and dried. The obtained carboxylic acid crude crystals were dissolved in DMF (2.18 ml), WSC (0.167 g) and ammonium 1H-1,2,3-benzotriazol-1-olate (0.166 g) were added, and the mixture was stirred at room temperature for 45 min and further at 60° C. for 1 hr. The reaction mixture was purified by reversed-phase preparative HPLC (Gilson, Inc. UniPoint system, YMC ODS column 30×75 millimeter, 0.1% TFA containing acetonitrile-water [5:95-100:0]) to give the title compound (13.6 mg).

MS (ESI+, m/e) 426 (M+1)

¹H-NMR (DMSO-d₆) δ: 2.35 (3H, s), 2.44 (3H, s), 4.49 (2H, d), 7.31 (1H, brs), 7.39 (2H, d), 7.53 (1H, dd), 7.59 (1H, d), 7.65 (2H, d), 7.69 (1H, brs), 7.85 (1H, d), 7.93 (1H, brs), 7.98 (1H, brs), 8.30 (1H, t).

### Reference Example 29

### 2-chloro-4-(3,5-dimethyl-1H-pyrazol-1-yl)benzonitrile

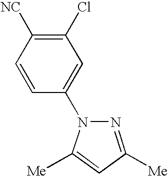

3,5-Dimethylpyrazole (15.0 g) was dissolved in DMF (100 mL), 60% sodium hydride (6.24 g) was added at 0° C. under ice-cooling, and the mixture was stirred for 30 min. 2-Chloro-4-fluorobenzonitrile (24.3 g) was added to the mixture, and the mixture was stirred at 0° C. for 1.5 hr. Water was added to the reaction mixture, and the obtained white precipitate was collected by filtration. The obtained solid was washed with water and hexane, and dried under reduced pressure to give the title compound (33.3 g).

¹H-NMR (CDCl₃) δ: 2.29 (3H, s), 2.42 (3H, s), 6.07 (1H, s), 7.51 (1H, dd), 7.73 (2H, d).

### Reference Example 30

### 4-(4-bromo-3,5-dimethyl-1H-pyrazol-1-yl)-2-chlorobenzonitrile

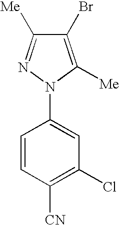

2-Chloro-4-(3,5-dimethyl-1H-pyrazol-1-yl)benzonitrile (7.30 g) synthesized in Reference Example 29 was dissolved in acetic acid (50 ml), bromine (5.54 g) was added dropwise slowly, and the mixture was stirred at room temperature for 30 min. The reaction mixture was diluted with water, and the obtained solid was collected by filtration and dried. The obtained solid was recrystallized from hexane-ethyl acetate to give the title compound (5.60 g) as colorless crystals.

¹H-NMR (CDCl₃) δ: 2.30 (3H, s), 2.42 (3H, s), 7.49 (1H, dd), 7.71 (1H, d), 7.76 (1H, d).

### Reference Example 31

### 2-chloro-4-(4-iodo-3,5-dimethyl-1H-pyrazol-1-yl)benzonitrile

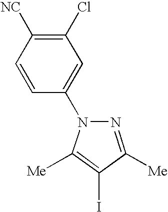

A mixture of 2-chloro-4-(3,5-dimethyl-1H-pyrazol-1-yl)benzonitrile (5.0 g) obtained in Reference Example 29, N-iodosuccinimide (NIS, 4.86 g) and acetonitrile (100 mL) were stirred at room temperature for 6 days. The reaction mixture was concentrated under reduced pressure. The residue was purified by column chromatography (hexane-ethyl acetate), and the obtained solid was washed with diisopropyl ether to give the title compound (5.05 g).

¹H-NMR (CDCl₃) δ: 2.31 (3H, s), 2.45 (3H, s), 7.48 (1H, dd,), 7.70 (1H, d), 7.76 (1H, d).

### Reference Example 32

### 2-fluoro-4-(4-iodo-3,5-dimethyl-1H-pyrazol-1-yl)benzonitrile

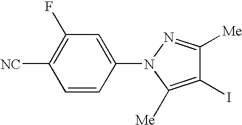

Using 3,5-dimethyl-4-iodopyrazole and 2,4-difluorobenzonitrile as starting materials and in the same manner as in Reference Example 29, the title compound was obtained.

¹H-NMR (CDCl₃) δ: 2.31 (3H, s), 2.47 (3H, s), 7.36-7.47 (2H, m), 7.72 (1H, dd).

### Reference Example 33

### 4-(4-acetyl-3,5-dimethyl-1H-pyrazol-1-yl)-2-chlorobenzonitrile

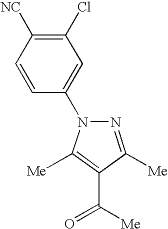

Using 4-acetyl-3,5-dimethylpyrazole and 2-chloro-4-fluorobenzonitrile as starting materials and in the same manner as in Reference Example 29, the title compound was obtained.

¹H-NMR (CDCl₃) δ: 2.51 (3H, s), 2.54 (3H, s), 2.61 (3H, s), 7.47 (1H, dd), 7.69 (1H, d), 7.80 (1H, d).

### Reference Example 34

### 4-(4-fluorobenzyl)-3,5-dimethyl-1H-pyrazole

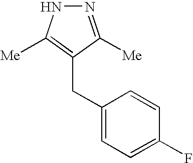

A mixture of acetylacetone (8.16 mL), a solution of 20% sodium ethoxide in ethanol (10.4 mL) and ethanol (55.9 mL) was heated to 50° C., and a solution of 4-fluorobenzyl bromide (5.00 g) in ethanol (26.5 mL) was added dropwise over 30 min. The reaction mixture was heated under reflux for 2 hr, allowed to cool to room temperature and concentrated under reduced pressure. The obtained residue was purified by column chromatography (hexane-ethyl acetate) to give a colorless oil (4.37 g). The oil was dissolved in ethanol (80 mL), hydrazine monohydrate (1.12 mL) was added, and the mixture was heated under reflux for 12 hr. The reaction mixture was allowed to cool to room temperature and concentrated under reduced pressure. The residue was recrystallized from toluene to give the title compound (2.96 g) as white crystals.

¹H-NMR (DMSO-d₆) δ: 1.98 (3H, s), 2.08 (3H, s), 3.63 (2H, s), 6.74-7.29 (4H, m), 11.98 (1H, brs).

### Reference Example 35

### methyl 2-[(3,5-dimethyl-1H-pyrazol-4-yl)methyl]benzoate

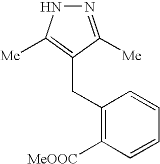

A mixture of acetyladetone (24.4 g), sodium ethoxide (5.53 g) and ethanol (150 mL) was heated to 50° C., and a solution of methyl 2-(chloromethyl)benzoate (15.0 g) in ethanol (150 mL) was added dropwise over 1 hr. The reaction mixture was heated under reflux for 4 hr, allowed to cool to room temperature and concentrated under reduced pressure. Water was added to the obtained residue, and the mixture was extracted twice with ethyl acetate. The organic layers were combined, washed with saturated brine, dried over anhydrous magnesium sulfate and concentrated. The residue was purified by column chromatography (hexane-ethyl acetate) to give a colorless oil (9.43 g). The oil was dissolved in ethanol (190 mL), hydrazine monohydrate (2.03 mL) was added, and the mixture was heated under reflux for 3 hr. The reaction mixture was allowed to cool to room temperature and concentrated under reduced pressure. The residue was purified by column chromatography (hexane-ethyl acetate) to give the title compound (5.10 g) as white crystals.

¹H-NMR (CDCl₃) δ: 2.08 (6H, s), 3.90 (3H, s), 4.12 (2H, s), 7.21-7.28 (2H, m), 7.32-7.40 (1H, m), 7.88 (1H, dd).

### Reference Example 36

### methyl 3-[(3,5-dimethyl-1H-pyrazol-4-yl)methyl]benzoate

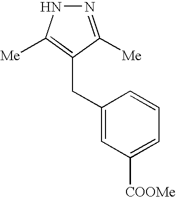

Using methyl 3-(chloromethyl)benzoate as a starting material and in the same manner as in Reference Example 35, the title compound was obtained.

¹H-NMR (CDCl₃) δ: 2.15 (6H, s), 3.78 (2H, s), 3.90 (3H, s), 7.27-7.38 (2H, m), 7.79-7.93 (2H, m).

### Reference Example 37

### methyl 4-[(3,5-dimethyl-1H-pyrazol-4-yl)methyl]benzoate

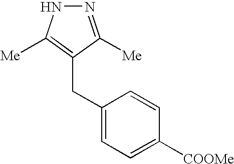

Using methyl 4-(bromomethyl)benzoate as a starting material and in the same manner as in Reference Example 35, the title compound was obtained.

¹H-NMR (CDCl₃) δ: 2.14 (6H, s), 3.79 (2H, s), 3.90 (3H, s), 7.17 (2H, d), 7.93 (2H, d), 9.47 (1H, brs).

### Reference Example 38

### 2-bromo-5-[(3,5-dimethyl-1H-pyrazol-4-yl)methyl]pyridine

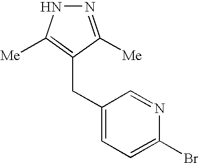

### Step 1

A solution of (6-bromopyridin-3-yl)methanol (2.39 g) described in a reference (Ellingboe, J. W. et al. J. Med. Chem. 1994, 37, 542-550.) in THF (65 mL) was ice-cooled, thionyl chloride (4.64 mL) was added, and the mixture was stirred at 0° C. for 6 hr. The reaction mixture was concentrated under reduced pressure. Toluene was added and the mixture was concentrated again, and dried under reduced pressure to give 2-bromo-5-(chloromethyl)pyridine hydrochloride (2.54 g) as a brown solid.

### Step 2

A mixture of 2-bromo-5-(chloromethyl)pyridine hydrochloride (2.00 g) obtained in step 1, acetylacetone (2.54 mL), sodium ethoxide (2.24 g), sodium iodide (1.23 g) and ethanol (20.0 mL) was heated at 50° C. for 17 hr. The reaction mixture was allowed to cool and concentrated under reduced pressure. The residue was neutralized with 0.1 mol/l hydrochloric acid and extracted twice with ethyl acetate. The organic layers were combined, washed with saturated brine, dried over anhydrous magnesium sulfate and concentrated. The obtained residue was purified by column chromatography (hexane-ethyl acetate) to give 3-[(6-bromopyridin-3-yl)methyl]pentane-2,4-dione (1.72 g) as a pale-yellow oil.

### Step 3

3-[(6-Bromopyridin-3-yl)methyl]pentane-2,4-dione (1.72 g) obtained in step 2 was dissolved in acetic acid (20 mL), hydrazine monohydrate (1.55 mL) was added, and the mixture was stirred at room temperature for 14 hr. The reaction mixture was concentrated under reduced pressure. The residue was neutralized with saturated aqueous sodium hydrogen carbonate solution and extracted twice with ethyl acetate. The organic layers were combined, washed with saturated brine, dried over anhydrous magnesium sulfate and concentrated. The obtained residue was purified by column chromatography (hexane-ethyl acetate) to give the title compound (1.42 g) as a pale-yellow solid.

¹H-NMR (CDCl₃) δ: 2.15 (6H, s), 3.66-3.73 (2H, m), 7.19-7.25 (1H, m), 7.29-7.40 (1H, m), 8.14-8.24 (1H, m).

### Reference Example 39

### methyl 4-[(3,5-diethyl-1H-pyrazol-4-yl)methyl]benzoate

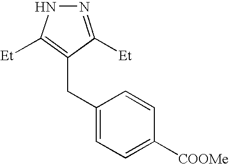

Heptane-3,5-dione (4.20 g) was dissolved in cyclopentyl methyl ether (25 mL), and the mixture was heated to 50° C. tert-Butoxy potassium (1.47 g) was added to the solution, and the mixture was stirred at 50° C. for 30 min. Methyl 4-(bromomethyl)benzoate (2.50 g) was added to the mixture, and the mixture was heated under reflux for 2 hr. The reaction mixture was allowed to cool to room temperature, saturated aqueous ammonium chloride solution (1 mL) and water (25 mL) were added, and the mixture was extracted with ethyl acetate. The organic layer was washed with saturated brine, dried over anhydrous magnesium sulfate and concentrated under reduced pressure. The obtained pale-yellow oil (3.58 g) was dissolved in acetic acid (7.2 mL), hydrazine monohydrate (0.693 mL) was added under ice-cooling, and the mixture was stirred at room temperature for 1 hr. The reaction mixture was concentrated under reduced pressure. The residue was neutralized with saturated aqueous sodium hydrogen carbonate, and the mixture was extracted with ethyl acetate. The organic layer was washed with saturated brine, dried over anhydrous magnesium sulfate and concentrated under reduced pressure. The residue was purified by column chromatography (hexane-ethyl acetate) to give the title compound (3.24 g) as a pale-yellow oil.

¹H-NMR (CDCl₃) δ: 1.15 (6H, t), 2.52 (4H, q), 3.82 (2H, s), 3.90 (3H, s), 7.17 (2H, d), 7.84-8.00 (2H, m), 9.49 (1H, brs).

### Reference Example 40

### 2-chloro-4-hydrazinylbenzonitrile hydrochloride

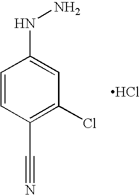

2-Chloro-4-fluorobenzonitrile (16.27 g) was dissolved in ethanol (65 mL), hydrazine monohydrate (7.65 mL) was added, and the mixture was stirred at 80° C. for 4 hr. Water (160 mL) was added, and the precipitate was collected by filtration. The precipitate was suspended in diethyl ether (325 mL), and hydrogen chloride-diethyl ether solution (1 mol/L, 81 mL) was added. After stirring for 30 min, insoluble material was collected by filtration to give the title compound (15.66 g) as a pale-yellow powder.

¹H-NMR (DMSO-d₆) δ: 6.95 (1H, dd), 7.17 (1H, d), 7.78 (1H, d), 9.29 (1H, s), 10.03 (3H, brs).

### Reference Example 41

### ethyl(2Z)-2-(methoxyimino)-4-oxopentanoate

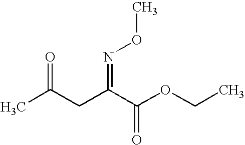

Ethyl 2,4-dioxopentanoate (10.0 g) was dissolved in ethanol (90 mL) and water (45 mL), a solution (45 mL) of N,O-dimethylhydroxylamine hydrochloride (4.76 g) in water was added dropwise, and the mixture was stirred at room temperature for 12 hr. Ethanol was removed under reduced pressure, water (60 mL) was added, and the mixture was extracted with ethyl acetate. The organic layer was washed with saturated brine, dried over anhydrous sodium sulfate and concentrated to give the title compound (11.41 g) as an orange oil.

¹H-NMR (CDCl₃) δ: 1.35 (3H, t), 2.21 (3H, s), 3.72 (2H, s), 4.07 (3H, s), 4.34 (2H, q).

### Reference Example 42

### tert-butyl 4-[(3Z)-2-acetyl-4-ethoxy-3-(methoxyimino)-4-oxobutyl]benzoate

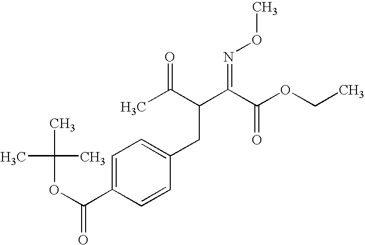

A suspension (30 mL) of ethyl (2Z)-2-(methoxyimino)-4-oxopentanoate (1.00 g) obtained in Reference Example 41, tert-butyl 4-(bromomethyl)benzoate (1.45 g) and potassium carbonate (0.88 g) in DMF was stirred at room temperature for 24 hr. The reaction mixture was neutralized with dilute hydrochloric acid, and the mixture was extracted with ethyl acetate. The organic layer was washed with saturated brine, dried over anhydrous sodium sulfate and concentrated. The residue was purified by column chromatography (hexane-ethyl acetate) to give the title compound (517.6 mg) as a yellow oil.

¹H-NMR (CDCl₃) δ: 1.23 (3H, t), 1.57 (9H, s), 2.08 (3H, s), 3.00 (1H, dd), 3.41 (1H, dd), 4.00 (3H, s), 4.13-4.29 (3H, m), 7.12-7.22 (2H, m), 7.83-7.92 (1H, m), 7.86 (1H, d).

### Reference Example 43

### ethyl 4-[4-(tert-butoxycarbonyl)benzyl]-5-methyl-1H-pyrazole-3-carboxylate

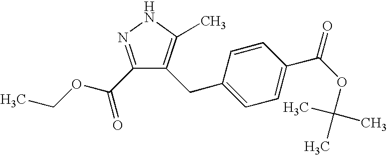

tert-Butyl 4-[(3Z)-2-acetyl-4-ethoxy-3-(methoxyimino)-4-oxobutyl]benzoate (0.20 g) obtained in Reference Example 42 and hydrazine monohydrate (0.077 mL) were dissolved in acetic acid (2 mL), and the mixture was stirred at 80° C. for 1 hr. The reaction mixture was neutralized with aqueous sodium hydrogen carbonate solution, and the mixture was extracted with ethyl acetate. The organic layer was washed with saturated brine, dried over anhydrous sodium sulfate and concentrated. The residue was purified by column chromatography (hexane-ethyl acetate) to give the title compound (120.6 mg) as a pale-yellow oil.

¹H-NMR (CDCl₃) δ: 1.33 (3H, t), 1.57 (9H, s), 2.19 (3H, s), 4.14 (2H, s), 4.29-4.39 (2H, m), 7.19 (2H, d), 7.87 (2H, d).

### Reference Example 44

### 4-{[1-(4-bromo-3-chlorophenyl)-3,5-dimethyl-1H-pyrazol-4-yl]methyl}-N-(2-hydroxy-2-methylpropyl)benzamide

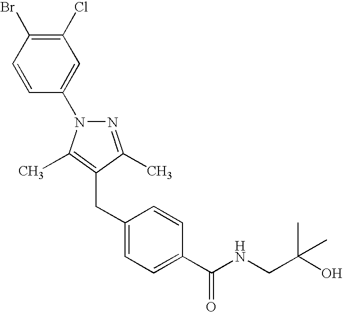

A solution of methyl 4-[(3,5-dimethyl-1H-pyrazol-4-yl)methyl]benzoate (1.00 g) synthesized in Reference Example 37, 1-bromo-2-chloro-4-fluorobenzene (8.60 g) and potassium carbonate (0.735 g) in DMSO (15 mL) was stirred at 220° C. for 40 min under microwave irradiation. The reaction mixture was allowed to cool and mixed with saturated aqueous ammonium chloride solution, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) to give methyl 4-{[1-(4-bromo-3-chlorophenyl)-3,5-dimethyl-1H-pyrazol-4-yl]methyl}benzoate (666 mg) and 4-{[1-(4-bromo-3-chlorophenyl)-3,5-dimethyl-1H-pyrazol-4-yl]methyl}benzoic acid ( 284 mg). To a solution of methyl 4-{[1-(4-bromo-3-chlorophenyl)-3,5-dimethyl-1H-pyrazol-4-yl]methyl}benzoate (93.6 mg) in THF (1 mL)-ethyl acetate (1 mL) were added lithium hydroxide monohydrate (18.1 mg) and water (1.00 mL), and the mixture was stirred at room temperature for 5 hr. The reaction mixture was mixed with 1 mol/l hydrochloric acid, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) to give 4-{[1-(4-bromo-3-chlorophenyl)-3,5-dimethyl-1H-pyrazol-4-yl]methyl}benzoic acid (85.0 mg). Using the compound (85.0 mg) and 1-amino-2-methyl-2-propanol as starting materials and in the same manner as in Example 424, the title compound (92.8 mg) was obtained as a colorless oil.

¹H-NMR (CDCl₃) δ: 1.22 (6H, s), 2.14 (3H, s), 2.20 (3H, s), 3.24 (1H, brs), 3.41 (2H, d), 3.79 (2H, s), 6.87 (1H, d), 7.12-7.24 (3H, m), 7.57 (1H, d), 7.62-7.75 (3H, m).

### Example 1

### 4-{4-[hydroxy(phenyl)methyl]-3,5-dimethyl-1H-pyrazol-1-yl}benzonitrile

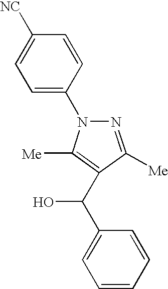

Using 4-(4-formyl-3,5-dimethyl-1H-pyrazol-1-yl)benzonitrile obtained in Reference Example 3 as a starting material and in the same manner as in Reference Example 4, the title compound was obtained.

¹H-NMR (CDCl₃) δ: 2.07 (1H, d), 2.20 (3H, s), 2.31 (3H, s), 5.96 (1H, d), 7.24-7.44 (5H, m), 7.53-7.62 (2H, m), 7.70-7.79 (2H, m).

### Example 2

### 4-(4-{[(4-fluorophenyl)sulfanyl]methyl}-3,5-dimethyl-1H-pyrazol-1-yl)benzonitrile

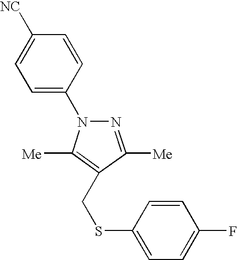

4-[4-(Hydroxymethyl)-3,5-dimethyl-1H-pyrazol-1-yl]benzonitrile (300 mg) obtained in Reference Example 7, 4-fluorothiophenol (152 mg), tri(n-butyl)phosphine (400 mg) and 1,1′-(azodicarbonyl)dipiperidine (500 mg) were dissolved in THF (10 ml), and the mixture was stirred at room temperature for 16 hr. The reaction mixture was diluted with ethyl acetate and aqueous sodium hydrogen carbonate solution. The organic layer was separated and dried over anhydrous magnesium sulfate, and the solvent was concentrated. The obtained residue was purified by column chromatography (ethyl acetate-hexane) and recrystallized from ethyl acetate-hexane to give the title compound (88 mg) as colorless crystals.

¹H-NMR (CDCl₃) δ: 2.09 (3H, s), 2.23 (3H, s), 3.85 (2H, s), 6.93-7.04 (2H, m), 7.29-7.38 (2H, m), 7.51-7.55 (2H, m), 7.69-7.78 (2H, m).

### Example 3

### 4-[3,5-dimethyl-4-(phenylcarbonyl)-1H-pyrazol-1-yl]benzonitrile

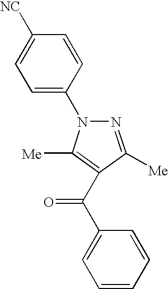

Using 4-{4-[hydroxy(phenyl)methyl]-3,5-dimethyl-1H-pyrazol-1-yl}benzonitrile obtained in Example 1 as a starting material and in the same manner as in Reference Example 5, the title compound was obtained.

¹H-NMR (CDCl₃) δ: 2.22 (3H, s), 2.36 (3H, s), 7.45-7.54 (2H, m), 7.55-7.68 (3H, m), 7.73-7.84 (4H, m).

### Example 4

### 4-[4-(1-hydroxy-1-phenylethyl)-3,5-dimethyl-1H-pyrazol-1-yl]benzonitrile

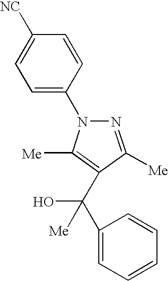

Using 4-[3,5-dimethyl-4-(phenylcarbonyl)-1H-pyrazol-1-yl]benzonitrile obtained in Example 3 and methylmagnesium bromide as starting materials and in the same manner as in Reference Example 4, the title compound was obtained.

¹H-NMR (CDCl₃) δ: 1.97 (3H, s), 2.06 (1H, s), 2.16 (3H, s), 2.19 (3H, s), 7.23-7.40 (3H, m), 7.41-7.49 (2H, m), 7.51-7.59 (2H, m), 7.70-7.78 (2H, m).

### Example 5

### 4-(4-{[(4-fluorophenyl)sulfonyl]methyl}-3,5-dimethyl-1H-pyrazol-1-yl)benzonitrile

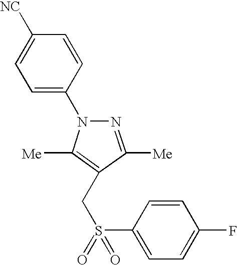

4-(4-{[(4-Fluorophenyl)sulfanyl]methyl}-3,5-dimethyl-1H-pyrazol-1-yl)benzonitrile (85 mg) obtained in Example 2 and 3-chloroperbenzoic acid (130 mg) were dissolved in DMF (5 ml), and the mixture was stirred at 0° C. for 3 hr. An aqueous sodium dithionite solution was added to the reaction mixture, and the mixture was further stirred for 30 min. The reaction mixture was diluted with ethyl acetate and aqueous sodium hydrogen carbonate solution. The organic layer was separated and dried over anhydrous magnesium sulfate, and the solvent was concentrated. The obtained residue was purified by column chromatography (ethyl acetate-hexane) and recrystallized from ethyl acetate-hexane to give the title compound (80 mg) as colorless crystals.

¹H-NMR (CDCl₃) δ: 1.93 (3H, s), 2.23 (3H, s), 4.17 (2H, s), 7.16-7.28 (2H, m), 7.50-7.61 (2H, m), 7.72-7.85 (4H, m).

### Example 6

### 4-(4-benzyl-3,5-dimethyl-1H-pyrazol-1-yl)benzonitrile

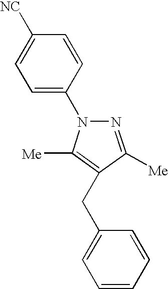

[1-(4-Cyanophenyl)-3,5-dimethyl-1H-pyrazol-4-yl](phenyl)methyl benzoate (240 mg) obtained in Example 7 and palladium carbon (10 mg) were suspended in THF (10 ml) under a hydrogen atmosphere, and the suspension was stirred at room temperature for 2 hr. Palladium carbon was filtered off, and the filtrate was concentrated under reduced pressure. The obtained residue was purified by column chromatography (ethyl acetate-hexane) and recrystallized from ethyl acetate-hexane to give the title compound (42 mg) as colorless crystals.

¹H-NMR (CDCl₃) δ: 2.20 (3H, s), 2.31 (3H, s), 3.80 (2H, s), 7.11-7.24 (3H, m), 7.24-7.33 (2H, m), 7.57-7.66 (2H, m), 7.70-7.78 (2H, m).

### Example 7

### [1-(4-cyanophenyl)-3,5-dimethyl-1H-pyrazol-4-yl](phenyl)methyl benzoate

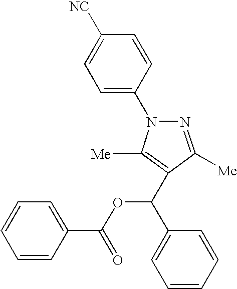

4-{4-[Hydroxy(phenyl)methyl]-3,5-dimethyl-1H-pyrazol-1-yl}benzonitrile (318 mg) obtained in Example 1 and 4-dimethylaminopyridine (6.4 mg) were dissolved in pyridine (10 ml), and the mixture was cooled to 5° C. Benzoyl chloride (0.13 ml) was added to the reaction mixture, and the mixture was stirred at room temperature for 16 hr. The reaction mixture was diluted with ethyl acetate and water, and the organic layer was separated. The organic layer was washed with aqueous sodium hydrogen carbonate solution and dried over anhydrous magnesium sulfate. The solvent was concentrated to give the title compound (317 mg) as colorless crystals.

¹H-NMR (CDCl₃) δ:2.25 (3H, s), 2.45 (3H, s), 7.22 (1H, s), 7.27-7.43 (5H, m), 7.45-7.54 (2H, m), 7.56-7.65 (3H, m), 7.71-7.79 (2H, m), 8.10-8.20 (2H, m).

### Example 8

### 4-[4-(1-hydroxy-1-phenylpropyl)-3,5-dimethyl-1H-pyrazol-1-yl]benzonitrile

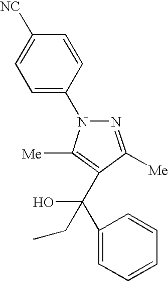

Using 4-[3,5-dimethyl-4-(phenylcarbonyl)-1H-pyrazol-1-yl]benzonitrile obtained in Example 3 and ethylmagnesium bromide as starting materials and in the same manner as in Reference Example 4, the title compound was obtained.

¹H-NMR (CDCl₃) δ: 0.95 (3H, t), 1.85-1.90 (1H, m), 2.12-2.45 (8H, m), 7.21-7.38 (3H, m), 7.39-7.47 (2H, m), 7.49-7.64 (2H, m), 7.69-7.84 (2H, m).

### Example 9

### 4-{4-[(E)-2-(4-fluorophenyl)ethenyl]-3,5-dimethyl-1H-pyrazol-1-yl}benzonitrile

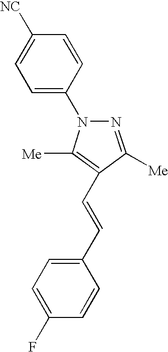

Sodium hydride (53.3 mg) was suspended in THF (10 ml), and the mixture was cooled to 5° C. Diethyl (4-fluorobenzyl)phosphonate (328 mg) was added, and the mixture was stirred for 15 min. 4-(4-Formyl-3,5-dimethyl-1H-pyrazol-1-yl)benzonitrile (200 mg) obtained in Reference Example 3 was added to the reaction mixture, and the mixture was further stirred at room temperature for 16 hr. The reaction mixture was diluted with ethyl acetate and aqueous sodium hydrogen carbonate solution. The organic layer was separated and dried over anhydrous magnesium sulfate, and the solvent was concentrated. The obtained residue was purified by column chromatography (ethyl acetate-hexane) and recrystallized from ethyl acetate-hexane to give the title compound (200 mg) as colorless crystals.

¹H-NMR (CDCl₃) δ: 2.46 (3H, s), 2.48 (3H, s), 6.74 (1H, d), 6.86 (1H, d), 6.99-7.12 (2H, m), 7.39-7.50 (2H, m), 7.57-7.66 (2H, m), 7.71-7.84 (2H, m).

### Example 10

### 4-{4-[(4-fluorophenyl)carbonyl]-3,5-dimethyl-1H-pyrazol-1-yl}benzonitrile

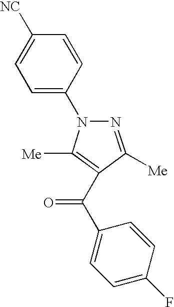

Using [1-(4-bromophenyl)-3,5-dimethyl-1H-pyrazol-4-yl](4-fluorophenyl)methanone obtained in Reference Example 8 as a starting material and in the same manner as in Reference Example 3, the title compound was obtained.

¹H-NMR (CDCl₃) δ: 2.22 (3H, s), 2.38 (3H, s), 7.11-7.23 (2H, m), 7.60-7.68 (2H, m), 7.77-7.89 (4H, m).

### Example 11

### 4-(4-{1-[(4-fluorophenyl)sulfanyl]ethyl}-3,5-dimethyl-1H-pyrazol-1-yl)benzonitrile

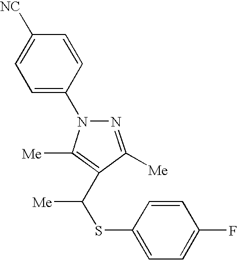

Using 4-[4-(1-hydroxyethyl)-3,5-dimethyl-1H-pyrazol-1-yl]benzonitrile obtained in Reference Example 9 as a starting material and in the same manner as in Example 2, the title compound was obtained.

¹H-NMR (CDCl₃) δ: 1.66 (3H, d), 2.08 (3H, s), 2.27-2.37 (3H, m), 4.16-4.52 (1H, m), 6.89-7.15 (2H, m), 7.23-7.36 (2H, m), 7.43-7.56 (2H, m), 7.67-7.81 (2H, m).

### Example 12

### methyl 4-benzyl-1-(4-cyanophenyl)-3-methyl-1H-pyrazol-5-yl carbonate

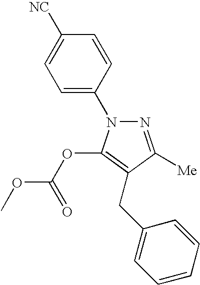

4-(4-Benzyl-3-methyl-5-oxo-4,5-dihydro-1H-pyrazol-1-yl)benzonitrile (150 mg) obtained in Reference Example 10 was dissolved in THF (20 ml), and the mixture was cooled to 5° C. Sodium hydride (31.1 mg) was added to the reaction mixture, and the mixture was stirred for 10 min. Methyl chlorocarbonate (0.08 ml) was added, and the mixture was further stirred for 3 hr. The reaction mixture was diluted with ethyl acetate and water. The organic layer was separated, and washed with water and saturated brine. The organic layer was dried over anhydrous magnesium sulfate, and the solvent was concentrated. The obtained residue was purified by column chromatography (ethyl acetate-hexane) and recrystallized from ethyl acetate-hexane to give the title compound (150 mg) as colorless crystals.

¹H-NMR (CDCl₃) δ: 2.17 (3H, s), 3.73 (2H, s), 3.78 (3H, s), 7.15-7.37 (5H, m), 7.67-7.79 (4H, m).

### Example 13

### 4-benzyl-1-(4-cyanophenyl)-3-methyl-1H-pyrazol-5-yl acetate

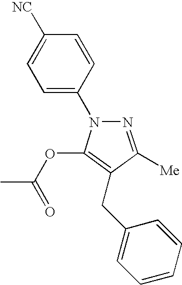

4-(4-Benzyl-3-methyl-5-oxo-4,5-dihydro-1H-pyrazol-1-yl)benzonitrile (150 mg) obtained in Reference Example 10 was dissolved in THF (20 ml), and the mixture was cooled to 5° C. Sodium hydride (31.1 mg) was added to the reaction mixture, and the mixture was stirred for 10 min. Acetic anhydride (0.147 ml) was added, and the mixture was further stirred for 3 hr. The reaction mixture was diluted with ethyl acetate and water, and the organic layer was separated, and washed with water and saturated brine. The organic layer was dried over anhydrous magnesium sulfate, and the solvent was concentrated. The obtained residue was purified by column chromatography (ethyl acetate-hexane) and recrystallized from ethyl acetate-hexane to give the title compound (125 mg) as pale-yellow crystals.

¹H-NMR (CDCl₃) δ: 2.10 (3H, s), 2.19 (3H, s), 3.69 (2H, s), 7.14-7.35 (5H, m), 7.64-7.78 (4H, m).

### Example 14

### 2-chloro-4-{4-[hydroxy(phenyl)methyl]-3,5-dimethyl-1H-pyrazol-1-yl}benzonitrile

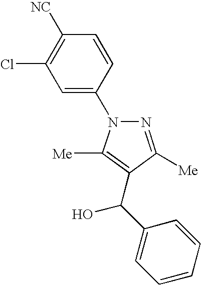

Using [1-(4-bromo-3-chlorophenyl)-3,5-dimethyl-1H-pyrazol-4-yl](phenyl)methanol obtained in Reference Example 14 as a starting material and in the same manner as in Reference Example 3, the title compound was obtained.

¹H-NMR (CDCl₃) δ: 2.13 (1H, d), 2.19 (3H, s), 2.34 (3H, s), 5.95 (1H, d), 7.26-7.43 (5H, m), 7.47 (1H, dd), 7.69 (1H, d), 7.73 (1H, d).

### Example 15

### 2-[1-(3-chloro-4-cyanophenyl)-3,5-dimethyl-1H-pyrazol-4-yl]-2-oxo-N-pyridin-3-yl acetamide

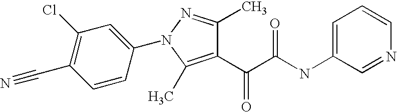

To a solution (0.240M, 2.50 ml) of 3-aminopyridine in dimethylacetamide was added dropwise a solution (0.200M, 2.50 ml) of [1-(3-chloro-4-cyanophenyl)-3,5-dimethyl-1H-pyrazol-4-yl](oxo)acetyl chloride synthesized in Reference Example 19 in dimethylacetamide, and the mixture was stirred at 60° C. for 18 hr. The reaction mixture was purified by reversed-phase preparative HPLC (Gilson, Inc. UniPoint system, YMC ODS column 30×75 millimeter, 0.1% TFA containing acetonitrile-water [5:95-100:0]) to give the title compound (37.2 mg).

MS (ESI+, m/e) 380 (M+1).

¹H-NMR (DMSO-d₆) δ: 2.37 (3H, s), 2.55 (3H, s), 7.44 (1H, dd), 7.82 (1H, dd), 8.07 (1H, d), 8.15 (1H, ddd), 8.20 (1H, d), 8.38 (1H, dd), 8.88 (1H, d), 11.18 (1H, s).

### Example 16

### 2-[1-(3-chloro-4-cyanophenyl)-3,5-dimethyl-1H-pyrazol-4-yl]-N-(4-fluorophenyl)-2-oxoacetamide

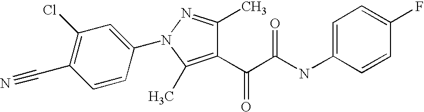

Using 4-fluoroaniline as a starting material and in the same manner as in Example 15, the title compound was obtained.

MS (ESI+, m/e) 397 (M+1).

¹H-NMR (DMSO-d₆) δ: 2.36 (3H, s), 2.55 (3H, s), 7.24 (2H, d), 7.74 (2H, dd), 7.81 (1H, dd), 8.07 (1H, d), 8.19 (1H, d), 10.98 (1H, s).

### Example 17

### 2-chloro-4-(4-{3-[(4-fluorophenyl)sulfonyl]-2-oxopropanoyl}-3,5-dimethyl-1H-pyrazol-1-yl)benzonitrile

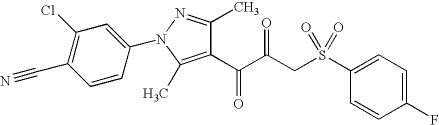

4-Fluorophenyl methyl sulfone (0.262 g) was dissolved in THF (3.75 ml), 1.57 mol/l n-butyllithium/hexane solution (1.05 ml) was added dropwise at −78° C. under a nitrogen atmosphere. The mixture was warmed to room temperature, and stirred for 10 min. The mixture was cooled again to −78° C., a solution (6.88 ml) of [1-(3-chloro-4-cyanophenyl)-3,5-dimethyl-1H-pyrazol-4-yl](oxo)acetyl chloride (0.443 g) synthesized in Reference Example 19 in THF was added dropwise at the same temperature for 30 min. The reaction temperature was allowed to warm to 0° C. over 2 hr, and the mixture was stirred at room temperature. 1 mol/l Hydrochloric acid (3.0 ml) was added to the reaction mixture, and the mixture was extracted with ethyl acetate. The organic layer was washed with water, 5% aqueous sodium bicarbonate and saturated brine, dried over anhydrous magnesium sulfate and concentrated. The residue was purified by reversed-phase preparative HPLC (Gilson, Inc. UniPoint system, YMC ODS column 30×75 millimeter, 0.1% TFA containing acetonitrile-water [5:95-100:0]) and concentrated. The residue was suspended in diisopropyl ether and collected by filtration to give the title compound (20.9 mg).

MS (ESI+, m/e) 460 (M+1)

¹H-NMR (DMSO-d₆) δ: 2.44 (3H, s), 2.55 (3H, s), 4.77 (2H, s), 7.31 (2H, dd), 7.50 (1H, dd), 7.71 (1H, dd), 7.83 (1H, d), 7.99 (2H, dd).

### Example 18

### 2-[1-(3-chloro-4-cyanophenyl)-3,5-dimethyl-1H-pyrazol-4-yl]-N-(4-fluorophenyl)-2-hydroxypropanamide

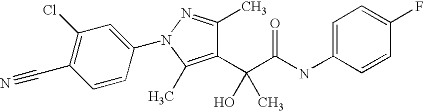

2-[1-(3-Chloro-4-cyanophenyl)-3,5-dimethyl-1H-pyrazol-4-yl]-N-(4-fluorophenyl)-2-oxoacetamide (32.1 mg) synthesized in Example 16 was dissolved in THF (3.24 ml), 3.0 mol/l methylmagnesium bromide/diethyl ether solution (0.809 ml) was added dropwise at 0° C., and the mixture was stirred at the same temperature for 30 min. A saturated aqueous ammonium chloride solution was added to the reaction mixture at 0° C., and the mixture was extracted with ethyl acetate. The organic layer was washed with saturated brine, dried over anhydrous magnesium sulfate and purified by silica gel column chromatography (ethyl acetate) to give the title compound (11.4 mg).

MS (ESI+, m/e) 413 (M+1)

¹H-NMR (DMSO-d₆) δ: 1.77 (3H, s), 2.30 (3H, s), 2.44 (3H, s), 6.28 (1H, brs), 7.14 (2H, dd), 7.65 (1H, dd), 7.76 (2H, dd), 7.87 (1H, d), 8.08 (1H, d), 9.83 (1H, brs).

### Example 19

### 2-chloro-4-(3,5-dimethyl-4-{[3-(trifluoromethyl)-1H-pyrazol-1-yl]methyl}-1H-pyrazol-1-yl)benzonitrile

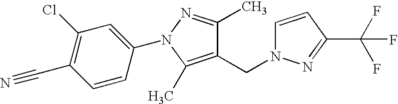

A resin reagent•PS-triphenylphosphine (about 171 mg) was added to a polypropylene tube equipped with a filter and a cap for preconditioning. Then, a solution (0.310M, 0.685 ml) of 3-(trifluoromethyl)pyrazole synthesized in Reference Example 21 in THF and a solution (0.200M, 0.685 ml) of 2-chloro-4-[4-(hydroxymethyl)-3,5-dimethyl-1H-pyrazol-1-yl]benzonitrile in THF were added, and the mixture was stirred. A solution (0.511M, 0.685 ml) of di-tert-butyl azodicarboxylate in THF was added, and the mixture was stirred at room temperature for 23 hr. The reaction mixture was filtered and washed to remove the resin reagent and the filtrate was concentrated. The residue was purified by reversed-phase preparative HPLC (Gilson, Inc. UniPoint system, YMC ODS column 30×75 millimeter, 0.1% TFA containing acetonitrile-water [5:95-100:0]) to give the title compound (8.2 mg).

MS (ESI+, m/e) 380 (M+1)

¹H-NMR (CDCl₃) δ: 2.29 (3H, s), 2.43 (3H, s), 5.20 (2H, s), 6.52 (1H, d), 7.33 (1H, dd), 7.50 (1H, dd), 7.72 (1H, d), 7.76 (1H, d).

### Example 20

### 2-chloro-4-(3,5-dimethyl-4-{[5-(trifluoromethyl)-1H-pyrazol-1-yl]methyl}-1H-pyrazol-1-yl)benzonitrile

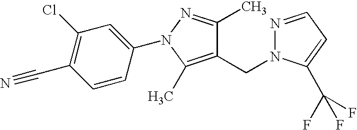

A resin reagent•PS-triphenylphosphine (about 171 mg) was added to a polypropylene tube equipped with a filter and a cap for preconditioning. Then, a solution (0.310M, 0.685 ml) of 3-(trifluoromethyl)pyrazole in THF and a solution (0.200M, 0.685 ml) of 2-chloro-4-[4-(hydroxymethyl)-3,5-dimethyl-1H-pyrazol-1-yl]benzonitrile synthesized in Reference Example 21 in THF were added, and the mixture was stirred. A solution (0.511M, 0.685 ml) of di-tert-butyl azodicarboxylate in THF was added, and the mixture was stirred at room temperature for 23 hr. The reaction mixture was filtered and washed to remove the resin reagent and the filtrate was concentrated. The residue was purified by reversed-phase preparative HPLC (Gilson, Inc. UniPoint system, YMC ODS column 30×75 millimeter, 0.1% TFA containing acetonitrile-water [5:95-100:0]) to give the title compound (13.8 mg).

MS (ESI+, m/e) 380 (M+1)

¹H-NMR (CDCl₃) δ: 2.23 (3H, s), 2.39 (3H, s), 5.25 (2H, s), 6.66 (1H, dd), 7.49 (1H, dd), 7.53 (1H, dd), 7.71 (1H, d), 7.75 (1H, d).

### Example 21

### N-[1-(3-chloro-4-cyanophenyl)-3,5-dimethyl-1H-pyrazol-4-yl]-4-fluorobenzamide

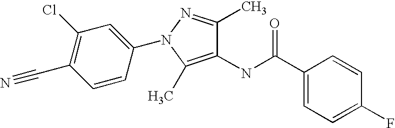

To a solution (0.200M, 0.325 ml) of 4-(4-amino-3,5-dimethyl-1H-pyrazol-1-yl)-2-chlorobenzonitrile synthesized in Reference Example 23 in dimethylacetamide was added a solution (0.312M, 0.325 ml) of 4-fluorobenzoyl chloride in dimethylacetamide, and the mixture was stirred with heating at 65° C. for 23 hr. The reaction mixture was purified by reversed-phase preparative HPLC (Gilson, Inc. UniPoint system, YMC ODS column 30×75 millimeter, 0.1% TFA containing acetonitrile-water [5:95-100:0]). The obtained fraction was concentrated to a half amount, and the obtained crystals were collected by filtration to give the title compound (16.5 mg).

MS (ESI+, m/e) 369 (M+1)

¹H-NMR (DMSO-d6) δ: 2.14 (3H, s), 2.34 (3H, s), 7.39-7.42 (2H, m), 7.79 (1H, dd), 7.99 (1H, d), 8.04-8.12 (3H, m), 9.82 (1H, brs).

### Example 22

### 1-(3-chloro-4-cyanophenyl)-N-(4-fluorophenyl)-3,5-dimethyl-1H-pyrazole-4-carboxamide

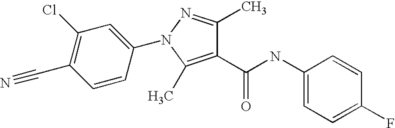

To a solution (0.240M, 1.05 ml) of 4-fluoroaniline in dimethylacetamide was added a solution (0.200M, 1.05 ml) of 1-(3-chloro-4-cyanophenyl)-3,5-dimethyl-1H-pyrazole-4-carbonyl chloride synthesized in Reference Example 20 in dimethylacetamide, and the mixture was stirred with heating at 60° C. for 16 hr. Water (2.0 ml) was added to the reaction mixture, and the mixture was extracted with ethyl acetate. The organic layer was washed with 5% aqueous sodium bicarbonate and saturated brine, dried over anhydrous magnesium sulfate and concentrated. The residue was purified by silica gel column chromatography (20-40% ethyl acetate/hexane), and the obtained fraction was concentrated and crystallized from diisopropyl ether to give the title compound (15.7 mg).

MS (ESI+, m/e) 369 (M+1)

¹H-NMR (DMSO-d₆) δ: 2.37 (3H, s), 2.52 (3H, s), 7.18 (2H, t), 7.70 (2H, dd), 7.76 (1H, dd), 7.98 (1H, d), 8.16 (1H, d), 9.98 (1H, brs).

### Example 23

### 1-(3-chloro-4-cyanophenyl)-N-(4-fluorobenzyl)-3,5-dimethyl-1H-pyrazole-4-carboxamide

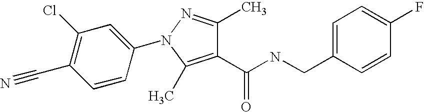

To a solution (0.240M, 1.05 ml) of 4-fluorobenzylamine in dimethylacetamide was added a solution (0.200M, 1.05 ml) of 1-(3-chloro-4-cyanophenyl)-3,5-dimethyl-1H-pyrazole-4-carbonyl chloride synthesized in Reference Example 20 in dimethylacetamide, and the mixture was stirred with heating at 60° C. for 16 hr. Water (2.0 ml) was added to the reaction mixture, and the mixture was extracted with ethyl acetate. The organic layer was washed with 5% aqueous sodium bicarbonate and saturated brine, dried over anhydrous magnesium sulfate and concentrated. The residue was purified by silica gel column chromatography (25-50% ethyl acetate/hexane), and the obtained fraction was concentrated and crystallized from diisopropyl ether to give the title compound (43.3 mg).

MS (ESI+, m/e) 383 (M+1)

¹H-NMR (DMSO-d₆) δ: 2.34 (3H, s), 2.49 (3H, s), 4.43 (2H, d), 7.17 (2H, t), 7.37 (2H, dd), 7.70 (1H, dd), 7.95 (1H, d), 8.13 (1H, d), 8.34 (1H, t).

### Example 24

### 2-[1-(3-chloro-4-cyanophenyl)-3,5-dimethyl-1H-pyrazol-4-yl]-N-(4-fluorobenzyl)-2-oxoacetamide

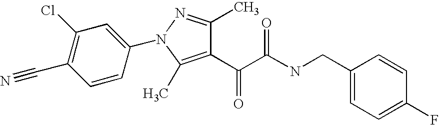

To a solution (0.240M, 1.05 ml) of 4-fluorobenzylamine in dimethylacetamide was added a solution (0.200M, 1.05 ml) of (1-[3-chloro-4-cyanophenyl]-3,5-dimethyl-1H-pyrazol-4-yl)(oxo)acetyl chloride synthesized in Reference Example 19 in dimethylacetamide, and the mixture was stirred with heating at 60° C. for 16 hr. Water (2.0 ml) was added to the reaction mixture, and the mixture was extracted with ethyl acetate. The organic layer was washed with 5% aqueous sodium bicarbonate and saturated brine, dried over anhydrous magnesium sulfate and concentrated. The residue was purified by silica gel column chromatography (25-50% ethyl acetate/hexane) to give the title compound (7.7 mg).

MS (ESI+, m/e) 411 (M+1)

¹H-NMR (CDCl₃) δ: 2.45 (3H, s), 2.54 (3H, s), 4.54 (2H, d), 7.05 (2H, t), 7.21 (1H, t), 7.32 (2H, dd), 7.50 (1H, dd), 7.71 (1H, d), 7.81 (1H, d).

### Example 25

### (±)-2-chloro-4-{4-[1-(4-chlorophenyl)-1-hydroxyethyl]-3,5-dimethyl-1H-pyrazol-1-yl}benzonitrile

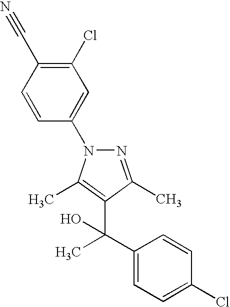

4-(4-Acetyl-3,5-dimethyl-1H-pyrazol-1-yl)-2-chlorobenzonitrile (0.100 g) synthesized in Reference Example 33 was suspended in THF (1.0 mL), 4-chlorophenylmagnesium bromide-THF solution (1 mol/l, 0.73 mL) was added under ice-cooling, and the mixture was stirred at 0° C. for 1 hr. A saturated aqueous ammonium chloride solution was added to the reaction mixture, and the mixture was extracted with ethyl acetate. The organic layer was washed with saturated brine, dried over anhydrous magnesium sulfate and concentrated. The obtained residue was purified by column chromatography (hexane-ethyl acetate) and further recrystallized from hexane-ethyl acetate to give the title compound (0.0776 g) as a white solid.

¹H-NMR (CDCl₃) δ:1.94 (3H, s), 2.02 (1H, s), 2.17 (3H, s), 2.22 (3H, s), 7.28-7.40 (4H, m), 7.43 (1H, dd), 7.66 (1H, d), 7.73 (1H, d).

### Example 26

### 2-chloro-4-{4-[1-(4-fluorophenyl)-1-hydroxyethyl]-3,5-dimethyl-1H-pyrazol-1-yl}benzonitrile

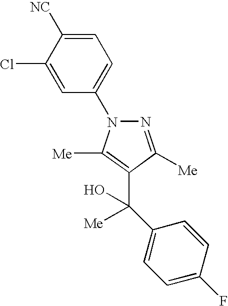

Using 1-[1-(4-bromo-3-chlorophenyl)-3,5-dimethyl-1H-pyrazol-4-yl]-1-(4-fluorophenyl)ethanol obtained in Reference Example 6 and methylmagnesium bromide as starting materials and in the same manner as in Reference Example 3, the title compound was obtained.

¹H-NMR(CDCl₃)δ: 1.95 (3H, s), 2.02 (1H, s), 2.14 (3H, s), 2.23 (3H, s), 6.97-7.08 (2H, m), 7.36-7.48 (3H, m), 7.66 (1H, d), 7.74 (1H, d).

### Example 27

### 2-chloro-4-[3,5-dimethyl-4-(1-phenylethenyl)-1H-pyrazol-1-yl]benzonitrile

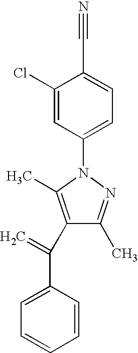

4-(4-Acetyl-3,5-dimethyl-1H-pyrazol-1-yl)-2-chlorobenzonitrile (0.100 g) synthesized in Reference Example 33 was suspended in THF (1.0 mL), phenylmagnesium bromide-THF solution (1M, 0.73 mL) was added under ice-cooling, and the mixture was stirred at 0° C. for 2 hr. A saturated aqueous ammonium chloride solution was added to the reaction mixture, and the mixture was extracted with ethyl acetate. The organic layer was washed with saturated brine, dried over anhydrous magnesium sulfate and concentrated. The obtained residue was purified by column chromatography (hexane-ethyl acetate) to give an oil (0.107 g). The oil was purified twice by reversed-phase high performance liquid chromatography (acetonitrile-water-trifluoroacetic acid) to give the title compound (0.0312 g) as a pale-yellow oil.

¹H-NMR (CDCl₃) δ:2.07 (3H, s), 2.27 (3H, s), 5.21 (1H, d), 5.82 (1H, d), 7.29-7.38 (5H, m), 7.56 (1H, dd), 7.71-7.83 (2H, m).

### Example 28

### 4-[4-(1-biphenyl-4-ylethenyl)-3,5-dimethyl-1H-pyrazol-1-yl]-2-chlorobenzonitrile

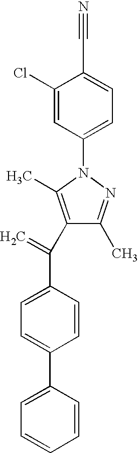

Using 4-(4-acetyl-3,5-dimethyl-1H-pyrazol-1-yl)-2-chlorobenzonitrile synthesized in Reference Example 33 and 4-biphenylmagnesium bromide as starting materials and in the same manner as in Example 27, the title compound was obtained.

¹H-NMR (CDCl₃) δ:2.12 (3H, s), 2.29 (3H, s), 5.25 (1H, s), 5.89 (1H, d), 7.32-7.50 (5H, m), 7.52-7.65 (5H, m), 7.72-7.82 (2H, m).

### Example 29

### 2-chloro-4-{4-[1-(3-fluorophenyl)-1-hydroxyethyl]-3,5-dimethyl-1H-pyrazol-1-yl}benzonitrile

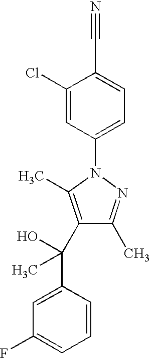

Using 4-(4-acetyl-3,5-dimethyl-1H-pyrazol-1-yl)-2-chlorobenzonitrile synthesized in Reference Example 33 and 3-fluorophenylmagnesium bromide as starting materials and in the same manner as in Example 25, the title compound was obtained.

¹H-NMR (CDCl₃) δ:1.95 (3H, s), 2.05 (1H, s), 2.18 (3H, s), 2.23 (3H, s), 6.92-7.03 (1H, m), 7.12-7.21 (2H, m), 7.29-7.37 (1H, m), 7.44 (1H, d), 7.67 (1H, s), 7.74 (1H, d).

### Example 30

### (±)-2-chloro-4-{4-[(4-fluorophenyl)(hydroxy)methyl]-3,5-dimethyl-1H-pyrazol-1-yl}benzonitrile

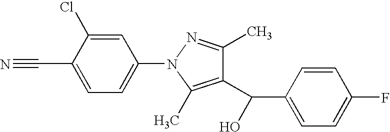

Using 2-chloro-4-(4-formyl-3,5-dimethyl-1H-pyrazol-1-yl)benzonitrile synthesized in Reference Example 17 and 4-fluorophenylmagnesium bromide as starting materials and in the same manner as in Example 25, the title compound was obtained.

¹H-NMR (CDCl₃) δ:2.03 (1H, d), 2.18 (3H, s), 2.34 (3H, s), 5.92 (1H, d), 7.05 (2H, dd), 7.36 (2H, dd), 7.48 (1H, dd), 7.70 (1H, d), 7.74 (1H, d).

### Example 31

### 4-{[1-(3-chloro-4-cyanophenyl)-3,5-dimethyl-1H-pyrazol-4-yl]methoxy}benzamide

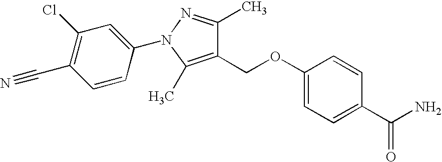

A resin reagent.PS-triphenylphosphine (about 190 mg) was added to a polypropylene tube equipped with a filter and a cap for preconditioning. Then, a solution (0.300M, 0.400 ml) of 4-hydroxybenzamide in THF and a solution (0.200M, 0.400 ml) of 2-chloro-4-[4-(hydroxymethyl)-3,5-dimethyl-1H-pyrazol-1-yl]benzonitrile obtained in Reference Example 21 in THF were added, and the mixture was stirred. A solution (0.500M, 0.400 ml) of di-tert-butyl azodicarboxylate in THF was added, and the mixture was stirred at room temperature for 22 hr. The reaction mixture was filtered and washed to remove the resin reagent and the filtrate was concentrated. The residue was purified by reversed-phase preparative HPLC (Gilson, Inc. UniPoint system, YMC ODS column 30×75 millimeter, 0.1% TFA containing acetonitrile-water [5:95-100:0]), and the obtained fraction was concentrated and crystallized from diisopropyl ether to give the title compound (14.8 mg).

MS (ESI+, m/e) 381(M+1)

¹H-NMR(DMSO-d₆) δ:2.24 (3H, s), 2.45 (3H, s), 5.01 (2H, s), 7.07 (2H, d), 7.19 (1H, brs), 7.77 (1H, dd), 7.84 (1H, brs), 7.86 (2H, d), 7.97 (1H, d), 8.11 (1H, d).

### Example 32

### methyl 4-[({[1-(3-chloro-4-cyanophenyl)-3,5-dimethyl-1H-pyrazol-4-yl]carbonyl}amino)methyl]benzoate

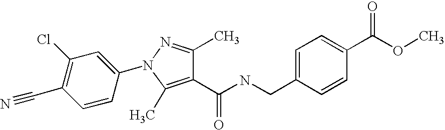

To a solution (5.00 ml) of methyl 4-(aminomethyl)benzoate (0.240M) and N,N-diisopropylethylamine (0.252M) in dimethylacetamide was added a solution (0.200M, 5.00 ml) of 1-(3-chloro-4-cyanophenyl)-3,5-dimethyl-1H-pyrazole-4-carbonyl chloride obtained in Reference Example 20 in dimethylacetamide, and the mixture was stirred with heating at 60° C. for 16 hr. 1 mol/l Hydrochloric acid (2.0 ml) was added to the reaction mixture, and the mixture was extracted with ethyl acetate. The organic layer was washed with 5% aqueous sodium bicarbonate and saturated brine, dried over anhydrous magnesium sulfate and concentrated. The residue was purified by silica gel column chromatography (25-65% ethyl acetate/hexane), and the obtained fraction was concentrated and crystallized from diisopropyl ether to give the title compound (38.8 mg).

MS (ESI+, m/e) 423(M+1)

¹H-NMR(DMSO-d₆) δ:2.36 (3H, s), 2.50 (3H, s), 3.85 (3H, s), 4.53 (2H, d), 7.47 (2H, d), 7.74 (1H, dd), 7.95 (2H, d), 7.96 (1H, d), 8.14 (1H, d), 8.41 (1H, t).

### Example 33

### (±)-4-[4-(1-biphenyl-4-yl-1-hydroxyethyl)-3,5-dimethyl-1H-pyrazol-1-yl]-2-chlorobenzonitrile

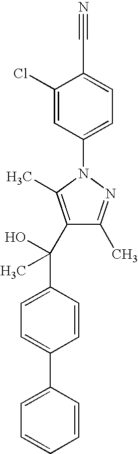

Using 4-(4-acetyl-3,5-dimethyl-1H-pyrazol-1-yl)-2-chlorobenzonitrile synthesized in Reference Example 33 and 4-biphenylmagnesium bromide as starting materials and in the same manner as in Example 25, the title compound was obtained.

¹H-NMR (CDCl₃) δ:2.00 (3H, s), 2.06 (1H, s), 2.19 (3H, s), 2.26 (3H, s), 7.33-7.77 (12H, m).

### Example 34

### (±)-2-chloro-4-[4-(1-hydroxy-1-methyl-2-phenylethyl)-3,5-dimethyl-1H-pyrazol-1-yl]benzonitrile

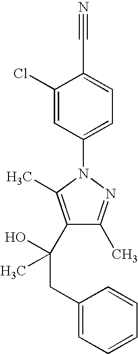

Using 4-(4-acetyl-3,5-dimethyl-1H-pyrazol-1-yl)-2-chlorobenzonitrile synthesized in Reference Example 33 and benzylmagnesium bromide as starting materials and in the same manner as in Example 25, the title compound was obtained.

¹H-NMR (CDCl₃) δ:1.65 (3H, s), 2.17 (3H, s), 2.28 (3H, s), 3.05 (2H, s), 7.02 (2H, dd), 7.22-7.29 (3H, m), 7.38 (1H, dd), 7.62 (1H, d), 7.73 (1H, d).

### Example 35

### 2-chloro-4-{4-[1-(4-fluorophenyl)ethenyl]-3,5-dimethyl-1H-pyrazol-1-yl}benzonitrile

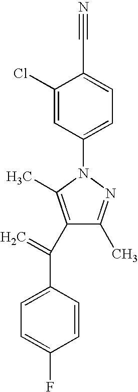

4-(4-Acetyl-3,5-dimethyl-1H-pyrazol-1-yl)-2-chlorobenzonitrile (0.100 g) synthesized in Reference Example 33 was suspended in THF (1.0 mL), 4-fluorophenylmagnesium bromide-THF solution (1 mol/l, 1.46 mL) was added under ice-cooling, and the mixture was stirred at 0° C. for 1 hr. A saturated aqueous ammonium chloride solution was added to the reaction mixture, and the mixture was extracted with ethyl acetate. The organic layer was washed with saturated brine, dried over anhydrous magnesium sulfate and concentrated. The obtained residue was dissolved in 10% hydrogen chloride methanol solution (4.0 mL), and the mixture was stirred at room temperature for 15 hr. The reaction mixture was concentrated under reduced pressure, neutralized with saturated aqueous sodium hydrogen carbonate solution, and extracted with ethyl acetate. The organic layer was washed with saturated brine, dried over anhydrous magnesium sulfate and concentrated. The obtained residue was purified by column chromatography (hexane-ethyl acetate) to give the title compound (0.101 g) as a white solid.

¹H-NMR (CDCl₃) δ:2.06 (3H, s), 2.27 (3H, s), 5.19 (1H, s), 5.75 (1H, s), 6.98-7.07 (2H, m), 7.28-7.36 (2H, m), 7.55 (1H, dd), 7.75 (1H, d), 7.78 (1H, d).

### Example 36

### 2-chloro-4-{4-[(4-fluorobenzyl)oxy]-3,5-dimethyl-1H-pyrazol-1-yl}benzonitrile

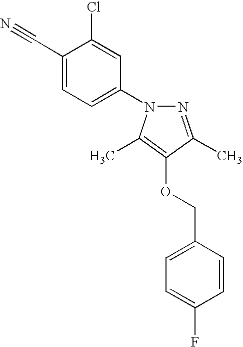

To a solution of 2-chloro-4-(4-hydroxy-3,5-dimethyl-1H-pyrazol-1-yl)benzonitrile (39.2 mg) synthesized in Reference Example 18 in DMF (2 ml) were added 1-(bromomethyl)-4-fluorobenzene (29.5 μL) and potassium carbonate (32.8 mg), and the mixture was stirred at 50° C. for 4 hr. The reaction mixture was ice-cooled and mixed with saturated aqueous ammonium chloride solution, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from ethyl acetate-hexane to give the title compound (27.9 mg) as yellow crystals.

¹H-NMR (CDCl₃) δ:2.20 (3H, s), 2.22 (3H, s), 4.85 (2H, s), 7.03-7.12 (2H, m), 7.32-7.39 (2H, m), 7.47 (1H, dd), 7.68-7.73 (2H, m).

### Example 37

### 4-{[1-(4-cyanophenyl)-3,5-dimethyl-1H-pyrazol-4-yl]methoxy}benzamide

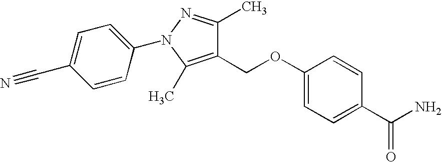

A resin reagent.PS-triphenylphosphine (457 mg) was added to a polypropylene tube equipped with a filter and a cap for preconditioning. Then, a solution (0.300M, 1.88 ml) of 4-hydroxybenzamide in THF and a solution (0.200M, 1.88 ml) of 4-[4-(hydroxymethyl)-3,5-dimethyl-1H-pyrazol-1-yl]benzonitrile obtained in Reference Example 26 in THF were added, and the mixture was stirred. A solution (0.500M, 1.88 ml) of di-tert-butyl azodicarboxylate in THF was added, and the mixture was stirred at room temperature for 3.5 hr. The reaction mixture was filtered and washed to remove the resin reagent and the filtrate was concentrated. The residue was purified by reversed-phase preparative HPLC (Gilson, Inc. UniPoint system, YMC ODS column 30×75 millimeter, 0.1% TFA containing acetonitrile-water [5:95-100:0]), and the obtained fraction was concentrated and crystallized from diisopropyl ether to give the title compound (48.5 mg).

MS (ESI+, m/e) 347(M+1)

¹H-NMR(DMSO-d₆) δ:2.24 (3H, s), 2.41 (3H, s), 5.01 (2H, s), 7.08 (2H, d), 7.19 (1H, brs), 7.77 (2H, d), 7.84 (1H, brs), 7.87 (2H, d), 7.99 (2H, d).

### Example 38

### 4-(3,5-dimethyl-4-{[5-(trifluoromethyl)-1H-pyrazol-1-yl]methyl}-1H-pyrazol-1-yl)benzonitrile

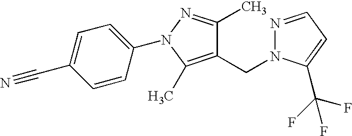

A resin reagent.PS-triphenylphosphine (662 mg) was added to a polypropylene tube equipped with a filter and a cap for preconditioning. Then, a solution (0.300M, 2.700 ml) of 3-(trifluoromethyl)pyrazole in THF and a solution (0.200M, 2.700 ml) of 4-[4-(hydroxymethyl)-3,5-dimethyl-1H-pyrazol-1-yl]benzonitrile obtained in Reference Example 26 in THF were added, and the mixture was stirred. A solution (0.500M, 2.700 ml) of di-tert-butyl azodicarboxylate in THF was added, and the mixture was stirred at room temperature for 3.5 hr. The reaction mixture was filtered and washed to remove the resin reagent and the filtrate was concentrated. The residue was purified by reversed-phase preparative HPLC (Gilson, Inc. UniPoint system, YMC ODS column 30×75 millimeter, 0.1% TFA containing acetonitrile-water [5:95-100:0]), and the obtained fraction was concentrated and crystallized from hexane-ethyl acetate to give the title compound (26.6 mg).

MS (ESI+, m/e) 346(M+1)

¹H-NMR(DMSO-d₆) δ:2.21 (3H, s), 2.44 (3H, s), 5.32 (2H, s), 6.71 (1H, d), 7.76 (2H, d), 7.97 (2H, d), 7.99-8.00 (1H, m).

### Example 39

### 4-(3,5-dimethyl-4-{[3-(trifluoromethyl)-1H-pyrazol-1-yl]methyl}-1H-pyrazol-1-yl)benzonitrile

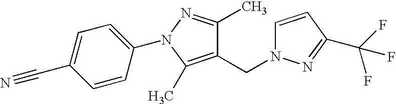

A resin reagent.PS-triphenylphosphine (662 mg) was added to a polypropylene tube equipped with a filter and a cap for preconditioning. Then, a solution (0.300M, 2.700 ml) of 3-(trifluoromethyl)pyrazole in THF and a solution (0.200M, 2.700 ml) of 4-[4-(hydroxymethyl)-3,5-dimethyl-1H-pyrazol-1-yl]benzonitrile obtained in Reference Example 26 in THF were added, and the mixture was stirred. A solution (0.500M, 2.700 ml) of di-tert-butyl azodicarboxylate in THF was added, and the mixture was stirred at room temperature for 3.5 hr. The reaction mixture was filtered and washed to remove the resin reagent and the filtrate was concentrated. The residue was purified by reversed-phase preparative HPLC (Gilson,. Inc. UniPoint system, YMC ODS column 30×75 millimeter, 0.1% TFA containing acetonitrile-water [5:95-100:0]). The obtained fraction was concentrated and further purified by silica gel column chromatography (ethyl acetate/hexane) to give the title compound (87.7 mg).

MS (ESI+, m/e) 346(M+1)

¹H-NMR(DMSO-d₆) δ:2.11 (3H, s), 2.36 (3H, s), 5.33 (2H, s), 6.92 (1H, d), 7.66 (1H, d), 7.74 (2H, d), 7.97 (2H, d).

### Example 40

### methyl N-[(4-{[1-(3-chloro-4-cyanophenyl)-3,5-dimethyl-1H-pyrazol-4-yl]oxy}phenyl)carbonyl]-2-amino-2-methylpropanoate

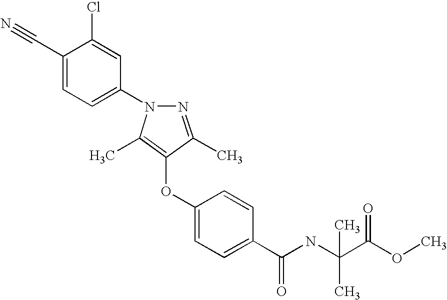

A solution of 4-{[1-(3-chloro-4-cyanophenyl)-3,5-dimethyl-1H-pyrazol-4-yl]oxy}benzoic acid (200 mg) synthesized in Example 306, 1-hydroxybenzotriazole (110 mg), methyl 2-amino-2-methylpropanoate hydrochloride (125 mg), N-(3-dimethylaminopropyl)-N′-ethylcarbodiimide hydrochloride (156 mg) and triethylamine (113 μL) in DMF (2 ml) was stirred at room temperature for 3 days. The reaction mixture was ice-cooled and mixed with 1 mol/l hydrochloric acid, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from ethyl acetate-hexane to give the title compound (142 mg) as white crystals.

¹H-NMR (CDCl₃) δ:1.54 (6H, s), 2.13 (3H, s), 2.31 (3H, s), 3.79 (3H, s), 6.72 (1H, brs), 6.92-6.98 (2H, m), 7.56 (1H, dd), 7.73-7.79 (3H, m), 7.80 (1H, d).

### Example 41

### 2-chloro-4-{4-[(E)-2-(4-fluorophenyl)ethenyl]-3,5-dimethyl-1H-pyrazol-1-yl}benzonitrile

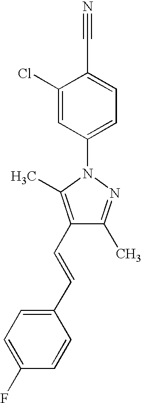

A mixture of 2-chloro-4-(4-iodo-3,5-dimethyl-1H-pyrazol-1-yl)benzonitrile (0.100 g) synthesized in Reference Example 31, 4-fluorostyrene (0.0501 mL), palladium acetate(II) (0.0031 g), sodium hydrogen carbonate (0.0588 g), tetrabutylammonium chloride (0.0778 g) and DMF (1.0 mL) was heated at 120° C. for 15 min and at 130° C. for 15 min in a tightly-sealed container under microwave irradiation. After cooling to room temperature, the reaction mixture was extracted twice with ethyl acetate. The organic layers were combined, washed with saturated brine, dried over anhydrous magnesium sulfate and concentrated. The obtained residue was purified by column chromatography (hexane-ethyl acetate), and the obtained resultant product was purified by reversed-phase high performance liquid chromatography (acetonitrile-water-trifluoroacetic acid) and further recrystallized from hexane-ethyl acetate to give the title compound (0.020 g) as a white solid.

¹H-NMR (CDCl₃) δ:2.45 (3H, s), 2.50 (3H, s), 6.79 (2H, dd), 7.06 (2H, t), 7.40-7.55 (3H, m), 7.72-7.79 (2H, m).

### Example 42

### (±)-4-[4-(1-biphenyl-4-ylethyl)-3,5-dimethyl-1H-pyrazol-1-yl]benzonitrile

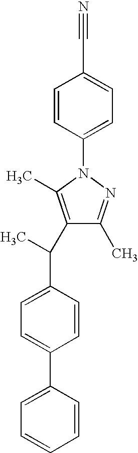

4-[4-(1-Biphenyl-4-ylethenyl)-3,5-dimethyl-1H-pyrazol-1-yl]-2-chlorobenzonitrile (0.089 g) synthesized in Example 28 was dissolved in ethanol (1.0 mL), 5% palladium carbon (0.0462 g) was added, and the mixture was stirred at room temperature for 2 hr under a hydrogen atmosphere (1 atm). The catalyst was filtered off, and the filtrate was concentrated under reduced pressure. The obtained residue was purified by column chromatography (hexane-ethyl acetate) to give two compounds of the title compound (0.0047 g) and 4-[4-(1-biphenyl-4-ylethyl)-3,5-dimethyl-1H-pyrazol-1-yl]-2-chlorobenzonitrile (0.050 g).

¹H-NMR (CDCl₃) δ:1.69 (3H, d), 2.19 (3H, s), 2.26 (3H, s), 4.20 (1H, q), 7.29-7.37 (3H, m), 7.39-7.47 (2H, m), 7.50-7.63 (6H, m), 7.71-7.78 (2H, m).

### Example 43

### (±)-4-[4-(1-biphenyl-4-ylethyl)-3,5-dimethyl-1H-pyrazol-1-yl]-2-chlorobenzonitrile

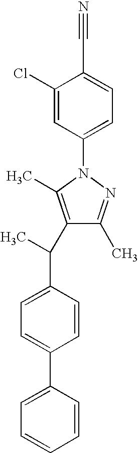

4-[4-(1-Biphenyl-4-ylethyl)-3,5-dimethyl-1H-pyrazol-1-yl]-2-chlorobenzonitrile (0.050 g) obtained by column chromatography in Example 42 was purified by reversed-phase high performance liquid chromatography (acetonitrile-water-trifluoroacetic acid) to give the title compound (0.0284 g) as a colorless oil.

¹H-NMR (CDCl₃) δ:1.69 (3H, d), 2.19 (3H, s), 2.27 (3H, s), 4.20 (1H, q), 7.28-7.38 (3H, m), 7.39-7.51 (3H, m), 7.52-7.62 (4H, m), 7.69 (1H, d), 7.74 (1H, d).

### Example 44

### (±)-2-chloro-4-{4-[1-(4-fluorophenyl)ethyl]-3,5-dimethyl-1H-pyrazol-1-yl}benzonitrile

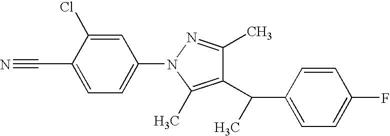

2-Chloro-4-{4-[1-(4-fluorophenyl)ethenyl]-3,5-dimethyl-1H-pyrazol-1-yl}benzonitrile (0.290 g) synthesized in Example 35 was dissolved in ethyl acetate (3.0 mL), 5% palladium carbon (0.175 g) was added, and the mixture was stirred at room temperature for 8 hr under a hydrogen atmosphere (1 atm). The catalyst was filtered off, and the filtrate was concentrated under reduced pressure. The obtained residue was dissolved in ethanol (3.0 mL), 5% palladium carbon (0.175 g) was added, and the mixture was stirred at room temperature for 5 hr under a hydrogen atmosphere (1 atm). 1 mol/l Aqueous sodium hydroxide solution (0.824 mL) was added to the reaction mixture, and the mixture was stirred at room temperature for 3 hr under a hydrogen atmosphere (1 atm). The catalyst was filtered off, and the filtrate was neutralized with 0.1 mol/l hydrochloric acid and concentrated under reduced pressure. Water was added to the obtained residue, and the mixture was extracted with ethyl acetate. The organic layer was washed with saturated brine, dried over anhydrous magnesium sulfate and concentrated. The obtained residue was purified by reversed-phase high performance liquid chromatography (acetonitrile-water-trifluoroacetic acid) to give 4-{4-[1-(4-fluorophenyl)ethyl]-3,5-dimethyl-1H-pyrazol-1-yl}benzonitrile (0.0488 g) and the title compound (0.0056 g).

¹H-NMR (CDCl₃) δ:1.63 (3H, d), 2.12 (3H, s), 2.25 (3H, s), 4.13 (1H, q), 6.95-7.04 (2H, m), 7.19 (2H, dd), 7.48 (1H, dd), 7.68-7.76 (2H, m).

### Example 45

### (±)-4-{4-[1-(4-fluorophenyl)ethyl]-3,5-dimethyl-1H-pyrazol-1-yl}benzonitrile

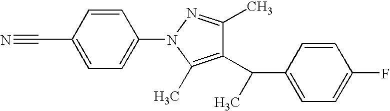

The title compound (0.0488 g) was obtained as a result of the reversed-phase high performance liquid chromatography in Example 44.

¹H-NMR (CDCl₃) δ:1.64 (3H, d), 2.13 (3H, s), 2.22 (3H, s), 4.13 (1H, q), 6.94-7.04 (2H, m), 7.21 (2H, dd), 7.55-7.61 (2H, m), 7.74 (2H, d).

### Example 46

### 2-chloro-4-[4-(4-fluorobenzyl)-3,5-dimethyl-1H-pyrazol-1-yl]benzonitrile

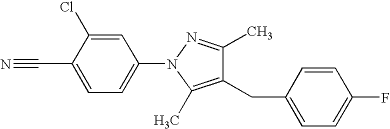

4-(4-Fluorobenzyl)-3,5-dimethyl-1H-pyrazole (0.100 g) synthesized in Reference Example 34 was dissolved in DMF (1.0 mL), 60% sodium hydride (0.0235 g) was added under ice-cooling, and the mixture was stirred for 15 min. 2-Chloro-4-fluorobenzonitrile (0.0941 g) was added to the reaction mixture, and the mixture was stirred at 0° C. for 1 hr. A saturated aqueous ammonium chloride solution was added to the reaction mixture, and the mixture was extracted twice with ethyl acetate. The organic layer was washed with saturated brine, dried over anhydrous magnesium sulfate and concentrated. The obtained residue was purified by column chromatography (hexane-ethyl acetate) to give the title compound (0.173 g) as a colorless oil.

¹H-NMR (CDCl₃) δ:2.18 (3H, s), 2.32 (3H, s), 3.76 (2H, s), 6.97 (2H, t), 7.05-7.13 (2H, m), 7.51 (1H, dd), 7.70-7.77 (2H, m).

### Example 47

### 1-(3-chloro-4-cyanophenyl)-N-(furan-3-ylmethyl)-3,5-dimethyl-1H-pyrazole-4-carboxamide

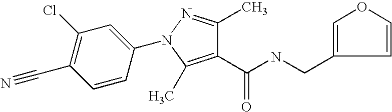

To a solution (0.180M, 0.625 ml) of 3-furylmethylamine in dimethylacetamide was added a solution (0.150M, 0.500 ml) of 1-(3-chloro-4-cyanophenyl)-3,5-dimethyl-1H-pyrazole-4-carbonyl chloride synthesized in Reference Example 20 in dimethylacetamide, and the mixture was stirred with heating at 60° C. for 18 hr. The reaction mixture was purified by reversed-phase preparative HPLC (Gilson, Inc. UniPoint system, YMC ODS column 30×75 millimeter, 0.1% TFA containing acetonitrile-water [5:95-100:0]) to give the title compound (20.0 mg).

MS (ESI+, m/e) 355(M+1)

### Example 48

### 1-(3-chloro-4-cyanophenyl)-3,5-dimethyl-N-(1H-pyrazol-3-ylmethyl)-1H-pyrazole-4-carboxamide

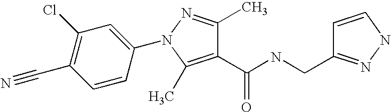

Using C-(1H-pyrazol-3-yl)methylamine as a starting material and in the same manner as in Example 47, the title compound was obtained.

MS (ESI+, m/e) 355(M+1).

### Example 49

### 1-(3-chloro-4-cyanophenyl)-N-(1H-imidazol-2-ylmethyl)-3,5-dimethyl-1H-pyrazole-4-carboxamide

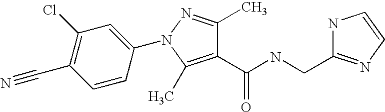

The title compound was obtained using C-(1H-imidazol-2-yl)-methylamine dihydrochloride as a starting material, adding 2 equivalents of 1-methylimidazole, and in the same manner as in Example 47.

MS (ESI+, m/e) 355(M+1).

### Example 50

### 1-(3-chloro-4-cyanophenyl)-N-(isoxazol-3-ylmethyl)-3,5-dimethyl-1H-pyrazole-4-carboxamide

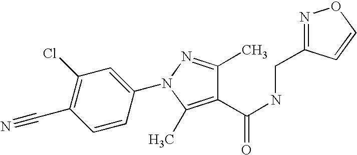

The title compound was obtained using C-isoxazol-3-yl-methylamine hydrochloride as a starting material, adding an equivalent of 1-methylimidazole, and in the same manner as in Example 47.

MS (ESI+, m/e) 356(M+1).

¹H-NMR (DMSO-d₆) δ:2.34 (3H, s), 2.50 (3H, s), 4.53 (2H, d), 6.52 (1H, d), 7.73 (1H, dd), 7.96 (1H, d), 8.14 (1H, d), 8.38 (1H, t), 8.86 (1H, d).

### Example 51

### 1-(3-chloro-4-cyanophenyl)-3,5-dimethyl-N-(1,3-oxazol-2-ylmethyl)-1H-pyrazole-4-carboxamide

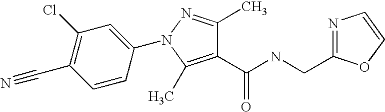

The title compound was obtained using oxazol-2-yl-methylamine hydrochloride as a starting material, adding an equivalent of 1-methylimidazole, and in the same manner as in Example 47.

MS (ESI+, m/e) 356(M+1).

### Example 52

### N-benzyl-1-(3-chloro-4-cyanophenyl)-3,5-dimethyl-1H-pyrazole-4-carboxamide

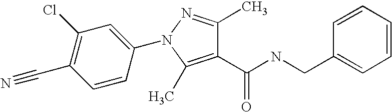

Using benzylamine as a starting material and in the same manner as in Example 47, the title compound was obtained.

MS (ESI+, m/e) 365(M+1).

### Example 53

### 1- (3-chloro-4-cyanophenyl)-3,5-dimethyl-N-(pyridin-3-ylmethyl)-1H-pyrazole-4-carboxamide

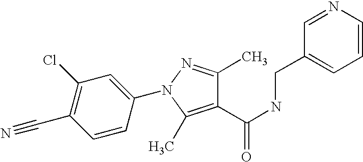

Using 3-(aminomethyl)pyridine as a starting material and in the same manner as in Example 47, the title compound was obtained.

MS (ESI+, m/e) 366(M+1).

### Example 54

### 1-(3-chloro-4-cyanophenyl)-3,5-dimethyl-N-(pyridin-4-ylmethyl)-1H-pyrazole-4-carboxamide

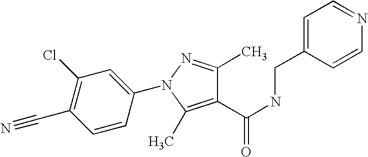

Using 4-picolylamine as a starting material and in the same manner as in Example 47, the title compound was obtained.

MS (ESI+, m/e) 366(M+l).

¹H-NMR (DMSO-d₆) δ:2.37 (3H, s), 2.51 (3H, s), 4.48 (2H, d), 7.32 (2H, d), 7.74 (1H, dd), 7.97 (1H, d), 8.14 (1H, d), 8.39 (1H, t), 8.52 (2H, d).

### Example 55

### 1- (3-chloro-4-cyanophenyl)-3,5-dimethyl-N-(thiophen-3-ylmethyl)-1H-pyrazole-4-carboxamide

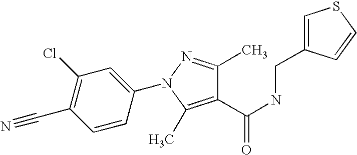

Using 3-thienylmethylamine as a starting material and in the same manner as in Example 47, the title compound was obtained.

MS (ESI+, m/e) 371(M+1).

### Example 56

### 1-(3-chloro-4-cyanophenyl)-3,5-dimethyl-N-(1,3-thiazol-2-ylmethyl)-1H-pyrazole-4-carboxamide

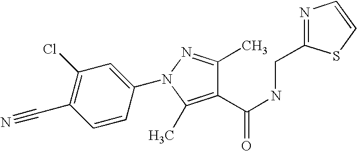

The title compound was obtained using C-thiazol-2-yl-methylamine hydrochloride as a starting material, adding an equivalent of 1-methylimidazole, and in the same manner as in Example 47.

MS (ESI+, m/e) 372(M+1).

### Example 57

### 1-(3-chloro-4-cyanophenyl)-3,5-dimethyl-N-[(2-oxo-1,2-dihydropyridin-3-yl)methyl]-1H-pyrazole-4-carboxamide

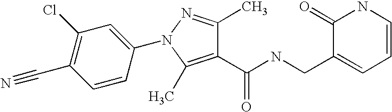

Using 3-(aminomethyl)-2-tert-butoxy pyridine as a starting material and in the same manner as in Example 47, the title compound was obtained.

MS (ESI+, m/e) 382(M+1).

¹H-NMR (DMSO-d₆) δ:2.36 (3H, s), 2.51 (3H, s), 4.21 (2H, d), 6.19 (1H, t), 7.31 (2H, d), 7.74 (1H, dd), 7.96 (1H, d), 8.06 (1H, t), 8.14 (1H, d), 11.66 (1H, brs).

### Example 58

### 1-(3-chloro-4-cyanophenyl)-N-(3-cyanobenzyl)-3,5-dimethyl-1H-pyrazole-4-carboxamide

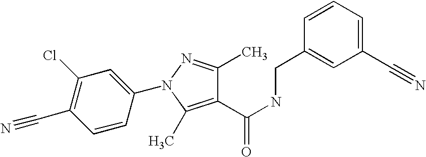

The title compound was obtained using 3-cyanobenzylamine hydrochloride as a starting material, adding an equivalent of lo 1-methylimidazole, and in the same manner as in Example 47.

MS (ESI+, m/e) 390(M+1).

### Example 59

4-[({[1-(3-chloro-4-cyanophenyl)-3,5-dimethyl-1H-pyrazol-4-yl]carbonyl}amino)methyl]benzoic acid

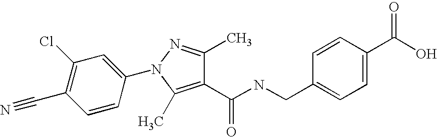

Using 4-(aminomethyl)benzoic acid as a starting material and in the same manner as in Example 47, the title compound was obtained.

MS (ESI+, m/e) 409(M+1).

### Example 60

### 1-(3-chloro-4-cyanophenyl)-N-(3,4-dichlorobenzyl)-3,5-dimethyl-1H-pyrazole-4-carboxamide

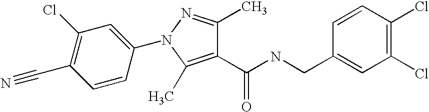

Using 3,4-dichlorobenzylamine as a starting material and in the same manner as in Example 47, the title compound was obtained.

MS (ESI+, m/e) 433(M+1).

¹H-NMR (DMSO-d₆) δ:2.35 (3H, s), 2.49 (3H, s), 4.44 (2H, d), 7.33 (1H, dd), 7.58 (1H, d), 7.62 (1H, d), 7.74 (1H, dd), 7.96 (1H, d), 8.14 (1H, d), 8.37 (1H, t).

### Example 61

### 1-(3-chloro-4-cyanophenyl)-3,5-dimethyl-N-{[6-(trifluoromethyl)pyridin-3-yl]methyl}-1H-pyrazole-4-carboxamide

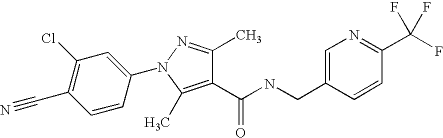

Using 3-aminomethyl-6-(trifluoromethyl)pyridine as a starting material and in the same manner as in Example 47, the title compound was obtained.

MS (ESI+, m/e) 434(M+1).

### Example 62

### 1-(3-chloro-4-cyanophenyl)-N-(4-cyanobenzyl)-3,5-dimethyl-1H-pyrazole-4-carboxamide

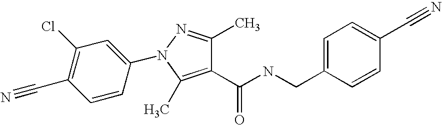

N-[4-(Aminocarbonyl)benzyl]-1-[4-(aminocarbonyl)-3-chlorophenyl]-3,5-dimethyl-1H-pyrazole-4-carboxamide (29.8 mg) synthesized in Reference Example 28 was dissolved in DMF (0.851 ml), and thionyl chloride (19.6 μl) was added dropwise at 0° C. under a nitrogen atmosphere. After stirring at room temperature for 40 min, thionyl chloride (19.6 μl) was further added dropwise, and the mixture was stirred at room temperature for 40 min. Saturated aqueous sodium bicarbonate (5.0 ml) was added to the reaction mixture, and the mixture was extracted with ethyl acetate and concentrated. The residue was purified by reversed-phase preparative HPLC (Gilson, Inc. UniPoint system, YMC ODS column 30×75 millimeter, 0.1% TFA containing acetonitrile-water [5:95-100:0]), concentrated and crystallized from diisopropyl ether to give the title compound (14.6 mg).

MS (ESI+, m/e) 390(M+1)

¹H-NMR(DMSO-d₆) δ:2.36 (3H, s), 2.50 (3H, s), 4.53 (2H, d), 7.52 (2H, d), 7.74 (1H, dd), 7.82 (2H, d), 7.96 (1H, d), 8.14 (1H, d), 8.41 (1H, t).

### Example 63

### 2-chloro-4-{4-[2-(4-fluorophenyl)ethyl]-3,5-dimethyl-1H-pyrazol-1-yl}benzonitrile

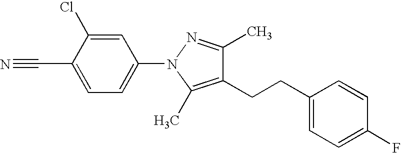

2-Chloro-4-{4-[(E)-2-(4-fluorophenyl)ethenyl]-3,5-dimethyl-1H-pyrazol-1-yl}benzonitrile (0.291 g) synthesized in Example 41 was dissolved in ethyl acetate (7.0 mL), 5% palladium carbon (0.352 g) was added, and the mixture was stirred at room temperature for 3 hr under a hydrogen atmosphere (1 atm). The catalyst was filtered off, and the filtrate was concentrated under reduced pressure. The obtained residue was purified by reversed-phase high performance liquid chromatography (acetonitrile-water-trifluoroacetic acid) to give 4-{4-[2-(4-fluorophenyl)ethyl]-3,5-dimethyl-1H-pyrazol-1-yl}benzonitrile (0.0309 g) and the title compound (0.101 g).

¹H-NMR (CDCl₃) δ:2.06 (3H, s), 2.17 (3H, s), 2.61-2.81 (4H, m), 6.91-7.00 (2H, m), 7.00-7.09 (2H, m), 7.43 (1H, dd), 7.65-7.74 (2H, m).

### Example 64

### 4-{4-[2-(4-fluorophenyl)ethyl]-3,5-dimethyl-1H-pyrazol-1-yl}benzonitrile

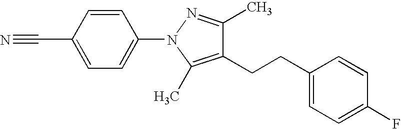

The title compound (0.0309 g) was obtained as a result of the reversed-phase high performance liquid chromatography in Example 63.

¹H-NMR (CDCl₃) δ:2.03 (3H, s), 2.18 (3H, s), 2.61-2.81 (4H, m), 6.91-7.09 (4H, m), 7.50-7.59 (2H, m), 7.69-7.77 (2H, m).

### Example 65

### 2-fluoro-4-{4-[(E)-2-(4-fluorophenyl)ethenyl]-3,5-dimethyl-1H-pyrazol-1-yl}benzonitrile

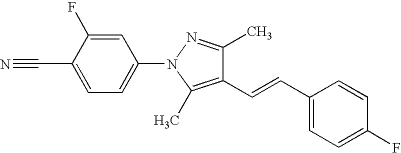

Using 2-fluoro-4-(4-iodo-3,5-dimethyl-1H-pyrazol-1-yl)benzonitrile synthesized in Reference Example 32 and 4-fluorostyrene as starting materials and in the same manner as in Example 41, the title compound was obtained.

¹H-NMR (CDCl₃) δ:2.41 (3H, s), 2.54 (3H, s), 6.83 (1H, d), 7.08 (1H, d), 7.19 (2H, t), 7.60-7.71 (3H, m), 7.78 (1H, dd), 8.03-8.12 (1H, m)

### Example 66

### 4-[4-(4-fluorobenzyl)-3,5-dimethyl-1H-pyrazol-1-yl]benzonitrile

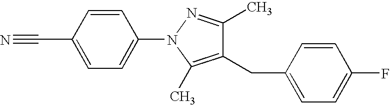

4-(4-Fluorobenzyl)-3,5-dimethyl-1H-pyrazole (0.200 g) synthesized in Reference Example 34 was dissolved in DMF (2.0 mL), 60% sodium hydride (0.047 g) was added under ice-cooling, and the mixture was stirred for 30 min. 4-Fluorobenzonitrile (0.237 g) was added to the reaction mixture, and the mixture was stirred at 0° C. for 2 hr and at room temperature for 18 hr. A saturated aqueous ammonium chloride solution was added to the reaction mixture, and the mixture was extracted twice with ethyl acetate. The organic layer was washed with saturated brine, dried over anhydrous magnesium sulfate and concentrated. The obtained residue was purified by column chromatography (hexane-ethyl acetate) and recrystallized from ethyl acetate-hexane to give the title compound (0.131 g) as a white solid.

¹H-NMR (CDCl₃) δ:2.19 (3H, s), 2.30 (3H, s), 3.76 (2H, s), 6.91-7.03 (2H, m), 7.05-7.16 (2H, m), 7.55-7.64 (2H, m), 7.70-7.78 (2H, m).

### Example 67

### 4-[4-(4-fluorobenzyl)-3,5-dimethyl-1H-pyrazol-1-yl]-2-(trifluoromethyl)benzonitrile

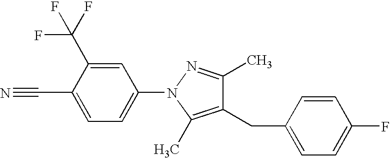

Using 4-(4-fluorobenzyl)-3,5-dimethyl-1H-pyrazole synthesized in Reference Example 34 and 2-trifluoromethyl-4-fluorobenzonitrile as starting materials and in the same manner as in Example 46, the title compound was obtained.

¹H-NMR (CDCl₃) δ:2.19 (3H, s), 2.35 (3H, s), 3.77 (2H, s), 6.91-7.03 (2H, m), 7.04-7.13 (2H, m), 7.75-7.82 (1H, m), 7.88-7.96 (1H, m), 8.02 (1H, d). Example 68

### 2-bromo-4-[4-(4-fluorobenzyl)-3,5-dimethyl-1H-pyrazol-1-yl]benzonitrile

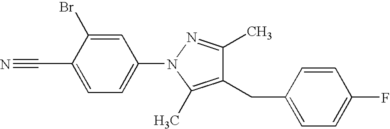

Using 4-(4-fluorobenzyl)-3,5-dimethyl-1H-pyrazole synthesized in Reference Example 34 and 2-bromo-4-fluorobenzonitrile as starting materials and in the same manner as in Example 46, the title compound was obtained.

¹H-NMR (CDCl₃) δ:2.18 (3H, s), 2.32 (3H, s), 3.76 (2H, s), 6.97 (2H, t), 7.04-7.14 (2H, m), 7.55 (1H, dd), 7.72 (1H, d), 7.91 (1H, d).

### Example 69

### 2-fluoro-4-{4-[2-(4-fluorophenyl)ethyl]-3,5-dimethyl-1H-pyrazol-1-yl}benzonitrile

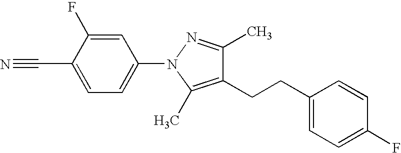

2-Fluoro-4-{4-[(E)-2-(4-fluorophenyl)ethenyl]-3,5-dimethyl-1H-pyrazol-1-yl}benzonitrile (0.130 g) synthesized in Example 65 was dissolved in ethanol (3.0 mL), 10% palladium carbon (0.041 g) was added, and the mixture was stirred at room temperature for 3 hr under a hydrogen atmosphere (1 atm). The catalyst was filtered off, and the filtrate was concentrated under reduced pressure. The obtained residue was purified by column chromatography (hexane-ethyl acetate) to give the title compound (0.024 g) as a white solid.

¹H-NMR (CDCl₃) δ:2.07 (3H, s), 2.17 (3H, s), 2.61-2.70 (2H, m), 2.69-2.79 (2H, m), 6.90-7.11 (4H, m), 7.31-7.43 (2H, m), 7.61-7.74 (1H, m).

### Example 70

### 1-(3-chloro-4-cyanophenyl)-N-[(6-cyanopyridin-3-yl)methyl]-3,5-dimethyl-1H-pyrazole-4-carboxamide

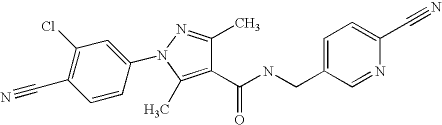

5-(Aminomethyl)pyridine-2-carbonitrile dihydrochloride (62.3 mg) and 1-methylimidazole (83 mg) were dissolved in dimethylacetamide (1.25 ml), a solution (0.200M, 1.25 ml) of 1-(3-chloro-4-cyanophenyl)-3,5-dimethyl-1H-pyrazole-4-carbonyl chloride synthesized in Reference Example 20 in dimethylacetamide was added, and the mixture was stirred at 60° C. for 23 hr. Water (2.0 ml) was added to the reaction mixture, and the mixture was extracted with ethyl acetate. The organic layer was washed with 5% aqueous sodium bicarbonate and saturated brine, dried over anhydrous magnesium sulfate and concentrated. The residue was purified by silica gel column chromatography (50-100% ethyl acetate/hexane), and the obtained fraction was concentrated and crystallized from ethyl acetate to give the title compound (73.0 mg).

MS (ESI+, m/e) 391(M+1)

¹H-NMR(DMSO-d₆) δ:2.36 (3H, s), 2.49 (3H, s), 4.56 (2H, d, J=5.8), 7.74 (1H, dd), 7.96 (1H, d), 7.96-8.05 (2H, m), 8.14 (1H, d), 8.43 (1H, t), 8.73 (1H, d).

### Example 71

### methyl 4-{[1-(3-chloro-4-cyanophenyl)-3,5-dimethyl-1H-pyrazol-4-yl]methyl}benzoate

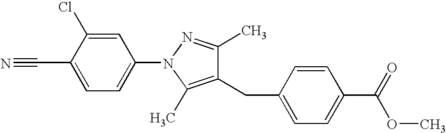

Using methyl 4-[(3,5-dimethyl-1H-pyrazol-4-yl)methyl]benzoate obtained in Reference Example 37 and 2-chloro-4-fluorobenzonitrile as starting materials and in the same manner as in Example 46, the title compound was obtained.

¹H-NMR (DMSO-d₆) δ:2.09 (3H, s), 2.39 (3H, s), 3.83 (3H, s), 3.87 (2H, s), 7.32 (2H, d), 7.71-7.78 (1H, m), 7.85-7.97 (3H, m), 8.08 (1H, d).

### Example 72

### 2-fluoro-4-[4-(4-fluorobenzyl)-3,5-dimethyl-1H-pyrazol-1-yl]benzonitrile

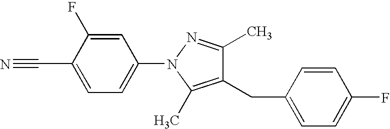

Using 4-(4-fluorobenzyl)-3,5-dimethyl-1H-pyrazole synthesized in Reference Example 34 and 2,4-difluorobenzonitrile as starting materials and in the same manner as in Example 46, the title compound was obtained.

¹H-NMR (CDCl₃) δ:2.18 (3H, s), 2.34 (3H, s), 3.76 (2H, s), 6.93-7.02 (2H, m), 7.04-7.12 (2H, m), 7.40-7.49 (2H, m), 7.69 (1H, dd).

### Example 73

### 3-fluoro-4-[4-(4-fluorobenzyl)-3,5-dimethyl-1H-pyrazol-1-yl]benzonitrile

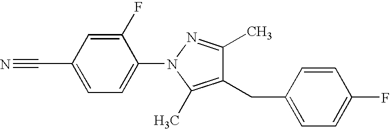

Using 4-(4-fluorobenzyl)-3,5-dimethyl-1H-pyrazole synthesized in Reference Example 34 and 3,4-difluorobenzonitrile as starting materials and in the same manner as in Example 46, the title compound was obtained.

¹H-NMR (CDCl₃) δ:2.12 (3H, d), 2.18 (3H, s), 3.77 (2H, s), 6.97 (2H, t), 7.06-7.15 (2H, m), 7.50-7.70 (3H, m).

### Example 74

### 3-chloro-4-[4-(4-fluorobenzyl)-3,5-dimethyl-1H-pyrazol-1-yl]benzonitrile

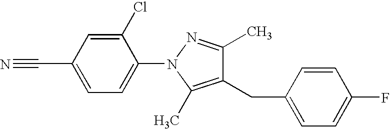

Using 4-(4-fluorobenzyl)-3,5-dimethyl-1H-pyrazole synthesized in Reference Example 34 and 3-chloro-4-fluorobenzonitrile as starting materials and in the same manner as in Example 46, the title compound was obtained.

¹H-NMR (CDCl₃) δ:2.04 (3H, s), 2.17 (3H, s), 3.77 (2H, s), 6.98 (2H, t), 7.05-7.14 (2H, m), 7.55 (1H, d), 7.66-7.73 (1H, m), 7.84 (1H, s).

### Example 75

### 4-{[1-(3-chloro-4-cyanophenyl)-3,5-dimethyl-1H-pyrazol-4-yl]methyl}benzamide

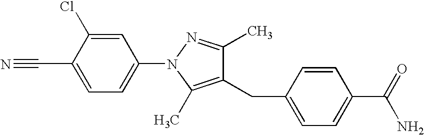

A mixture of 4-{[1-(3-chloro-4-cyanophenyl)-3,5-dimethyl-1H-pyrazol-4-yl]methyl}benzoic acid (0.100 g) synthesized in Example 92, N-[3-(dimethylamino)propyl]-N′-ethylcarbodiimide hydrochloride (0.0786 g), N-hydroxybenzotriazole ammonium salt (0.102 g) and DMF (1.0 mL) was stirred at room temperature for 15 hr. The reaction mixture was neutralized with saturated aqueous sodium hydrogen carbonate solution, and the mixture was extracted with ethyl acetate. The organic layer was washed with saturated brine, dried over anhydrous magnesium sulfate and concentrated. The obtained residue was purified by column chromatography (hexane-ethyl acetate), and the obtained solid was recrystallized from hexane-ethyl acetate to give the title compound (0.064 g) as a white solid.

¹H-NMR (DMSO-d₆) δ:2.10 (3H, s), 2.40 (3H, s), 3.83 (2H, s), 7.24 (2H, d), 7.29 (1H, brs), 7.71-7.82 (3H, m), 7.89 (1H, brs), 7.95 (1H, d), 8.08 (1H, d).

### Example 76

### N-(4-carbamoylbenzyl)-1-(3-chloro-4-cyanophenyl)-3,5-dimethyl-1H-pyrazole-4-carboxamide

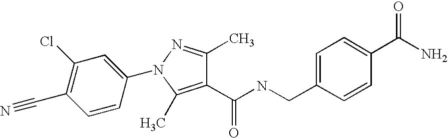

4-(Aminomethyl)-benzamide (0.916 g) and 1-methylimidazole (0.505 g) were dissolved in dimethylacetamide (25.0 ml), a solution (0.200M, 25.0 ml) of 1-(3-chloro-4-cyanophenyl)-3,5-dimethyl-1H-pyrazole-4-carbonyl chloride synthesized in Reference Example 20 in dimethylacetamide was added, and the mixture was stirred with heating at 60° C. for 1.5 hr. 1 mol/l Hydrochloric acid (40.0 ml) was added to the reaction mixture, and the mixture was extracted with ethyl acetate. The organic layer was washed with 5% aqueous sodium bicarbonate and saturated brine, dried over anhydrous magnesium sulfate and concentrated. The residue was purified by silica gel column chromatography (30-100% ethyl acetate/hexane), and the obtained fraction was concentrated and crystallized from ethyl acetate to give the title compound (0.539 g).

MS (ESI+, m/e) 408(M+1)

¹H-NMR(DMSO-d₆) δ:2.35 (3H, s), 2.50 (3H, s), 4.50 (2H, d), 7.32 (1H, brs), 7.39 (2H, d), 7.74 (1H, dd), 7.85 (2H, d), 7.93 (1H, brs), 7.96 (1H, d), 8.14 (1H, d), 8.38 (1H, t).

Elemental Analysis for C₂₁H₁₈ClN₅O₂.

Calcd.: C, 61.84; H, 4.45; N, 17.17.

Found: C, 61.66; H, 4.45; N, 17.18.

### Example 77

### 2-chloro-4-[4-(4-fluorophenoxy)-3,5-dimethyl-1H-pyrazol-1-yl]benzonitrile

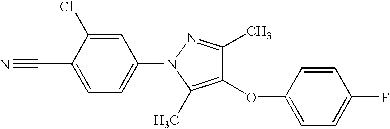

Using 4-(4-fluorophenoxy)-3,5-dimethyl-1H-pyrazole synthesized in Reference Example 27 and 2-chloro-4-fluorobenzonitrile as starting materials and in the same manner as in Example 46, the title compound was obtained.

¹H-NMR (CDCl₃) δ:2.13 (3H, s), 2.32 (3H, s), 6.82-6.91 (2H, m), 6.96-7.05 (2H, m), 7.55 (1H, dd), 7.72-7.81 (2H, m).

### Example 78

### 4-[4-(4-fluorophenoxy)-3,5-dimethyl-1H-pyrazol-1-yl]benzonitrile

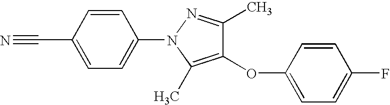

Using 4-(4-fluorophenoxy)-3,5-dimethyl-1H-pyrazole synthesized in Reference Example 27 and 4-fluorobenzonitrile as starting materials and in the same manner as in Example 66, the title compound was obtained.

¹H-NMR (CDCl₃) δ:2.14 (3H, s), 2.29 (3H, s), 6.84-6.92 (2H, m), 6.95-7.05 (2H, m), 7.63-7.70 (2H, m), 7.74-7.80 (2H, m).

### Example 79

### 4-{[1-(4-cyanophenyl)-3,5-dimethyl-1H-pyrazol-4-yl]methyl}benzamide

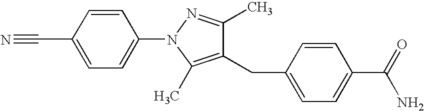

### Step 1

Methyl 4-[(3,5-dimethyl-1H-pyrazol-4-yl)methyl]benzoate (0.500 g) obtained in Reference Example 37 was dissolved in DMF (5.0 mL), 60% sodium hydride (0.0984 g) was added under ice-cooling, and the mixture was stirred for 30 min. 4-Fluorobenzonitrile (0.495 g) was added to the reaction mixture, and the mixture was stirred at room temperature for 24 hr. A saturated aqueous ammonium chloride solution was added to the reaction mixture, and the mixture was extracted twice with ethyl acetate. The organic layer was washed with saturated brine, dried over anhydrous magnesium sulfate and concentrated. The obtained residue was purified by column chromatography (hexane-ethyl acetate) to give methyl 4-{[1-(4-cyanophenyl)-3,5-dimethyl-1H-pyrazol-4-yl]methyl}benzoate (0.140 g) as a colorless oil.

### Step 2

Methyl 4-{[1-(4-cyanophenyl)-3,5-dimethyl-1H-pyrazol-4-yl]methyl}benzoate (0.140 g) obtained in step 1 was dissolved in a mixed solvent of THF (1.40 mL)-MeOH (1.40 mL), 1 mol/l aqueous sodium hydroxide solution (1.40 mL) was added, and the mixture was stirred at 50° C. for 3 hr. The reaction mixture was allowed to cool to room temperature, neutralized with 2 mol/l hydrochloric acid and concentrated under reduced pressure. The residue was mixed with water, and the mixture was extracted twice with ethyl acetate. The organic layer was washed with saturated brine, dried over anhydrous magnesium sulfate and concentrated. The obtained residue was purified by column chromatography (hexane-ethyl acetate) to give 4-{[1-(4-cyanophenyl)-3,5-dimethyl-1H-pyrazol-4-yl]methyl}benzoic acid (0.0418 g) as a white solid.

### Step 3

A mixture of 4-{[1-(4-cyanophenyl)-3,5-dimethyl-1H-pyrazol-4-yl]methyl}benzoic acid (0.0418 g) obtained in step 2, N-[3-(dimethylamino)propyl]-N′-ethylcarbodiimide hydrochloride (0.0362 g), N-hydroxybenzotriazole ammonium salt (0.0470 g) and DMF (1.0 mL) was stirred at room temperature for 13 hr. The reaction mixture was neutralized with saturated aqueous sodium hydrogen carbonate solution, and the mixture was extracted with ethyl acetate. The organic layer was washed with saturated brine, dried over anhydrous magnesium sulfate and concentrated. The obtained residue was purified by column chromatography (hexane-ethyl acetate) and further purified by reversed-phase high performance liquid chromatography (acetonitrile-water-trifluoroacetic acid) to give the title compound (0.0194 g) as a white solid.

¹H-NMR (CDCl₃) δ:2.19 (3H, s), 2.30 (3H, s), 3.85 (2H, s), 5.67-6.41 (2H, m), 7.23 (2H, d), 7.59-7.67 (2H, m), 7.71-7.78 (4H, m).

### Example 80

### methyl 4-({1-[4-cyano-3-(trifluoromethyl)phenyl]-3,5-dimethyl-1H-pyrazol-4-yl}methyl)benzoate

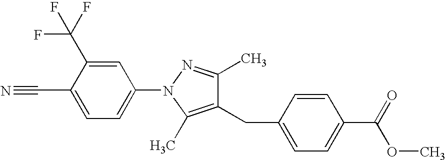

Using methyl 4-[(3,5-dimethyl-1H-pyrazol-4-yl)methyl]benzoate synthesized in Reference Example 37 and 2-trifluoromethyl-4-fluorobenzonitrile as starting materials and in the same manner as in Example 46, the title compound was obtained.

¹H-NMR (CDCl₃) δ:2.19 (3H, s), 2.35 (3H, s), 3.86 (2H, s), 3.91 (3H, s), 7.21 (2H, d), 7.75-7.83 (1H, m), 7.89-7.94 (1H, m), 7.97 (2H, d), 8.02 (1H, d).

### Example 81

### 4-({1-[4-cyano-3-(trifluoromethyl)phenyl]-3,5-dimethyl-1H-pyrazol-4-yl}methyl)benzoic acid

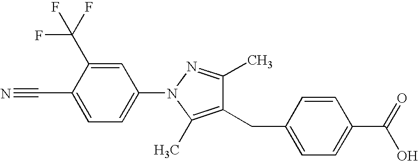

Methyl 4-({1-[4-cyano-3-(trifluoromethyl)phenyl]-3,5-dimethyl-1H-pyrazol-4-yl}methyl)benzoate (0.460 g) synthesized in Example 80 was dissolved in a mixed solvent of THF (4.6 mL)-methanol (4.6 mL), 1 mol/l aqueous sodium hydroxide solution (4.6 mL) was added dropwise slowly, and the mixture was stirred at room temperature for 5 hr. The reaction mixture was acidified with 2 mol/l hydrochloric acid, and the mixture was extracted twice with ethyl acetate. The organic layers were combined, washed with saturated brine, dried over anhydrous magnesium sulfate and concentrated to give a white solid (0.477 g).

¹H-NMR (DMSO-d₆) δ:2.11 (3H, s), 2.43 (3H, s), 3.88 (2H, s), 7.31 (2H, d), 7.87 (2H, d), 8.08 (1H, dd), 8.15 (1H, d), 8.29 (1H, d), 12.83 (1H, brs).

### Example 82

### 4-({1-[4-cyano-3-(trifluoromethyl)phenyl]-3,5-dimethyl-1H-pyrazol-4-yl}methyl)benzamide

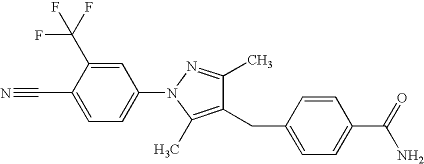

Using 4-({1-[4-cyano-3-(trifluoromethyl)phenyl]-3,5-dimethyl-1H-pyrazol-4-yl}methyl)benzoic acid synthesized in Example 81 as a starting material and in the same manner as in Example 75, the title compound was obtained.

¹H-NMR (CDCl₃) δ:2.19 (3H, s), 2.36 (3H, s), 3.86 (2H, s), 5.44-6.13 (2H, m), 7.22 (2H, d), 7.71-7.84 (3H, m), 7.89-7.97 (1H, m), 8.02 (1H, d).

### Example 83

### 4-{3,5-dimethyl-4-[4-(morpholin-4-ylcarbonyl)benzyl]-1H-pyrazol-1-yl}-2-(trifluoromethyl)benzonitrile

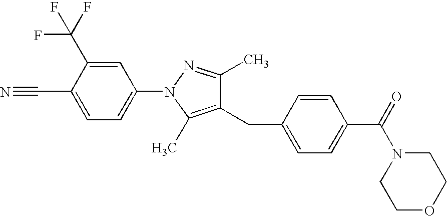

A mixture of 4-({1-[4-cyano-3-(trifluoromethyl)phenyl]-3,5-dimethyl-1H-pyrazol-4-yl}methyl)benzoic acid (0.0500 g) obtained in Example 81, N-[3-(dimethylamino)propyl]-N′-ethylcarbodiimide hydrochloride (0.0360 g), N-hydroxybenzotriazole (0.0254 g), morpholine (0.0164 mL) and DMF (0.5 mL) was stirred at room temperature for 4 days. 0.1 mol/l Hydrochloric acid was added to the reaction mixture, and the mixture was extracted twice with ethyl acetate. The organic layers were combined, washed with saturated brine, dried over anhydrous magnesium sulfate and concentrated. The obtained residue was purified by column chromatography (hexane-ethyl acetate) to give the title compound (0.0406 g) as a white solid.

¹H-NMR (CDCl₃) δ:2.20 (3H, s), 2.36 (3H, s), 3.31-3.93 (8H, m), 3.83 (2H, s), 7.18 (2H, d), 7.35 (2H, d), 7.76-7.83 (1H, m), 7.88-7.95 (1H, m), 8.02 (1H, s).

### Example 84

### 4-({1-[4-cyano-3-(trifluoromethyl)phenyl]-3,5-dimethyl-1H-pyrazol-4-yl}methyl)-N-(2-hydroxyethyl)benzamide

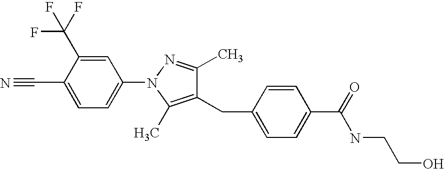

A mixture of 4-({1-[4-cyano-3-(trifluoromethyl)phenyl]-3,5-dimethyl-1H-pyrazol-4-yl}methyl)benzoic acid (0.050 g) synthesized in Example 81, 2-aminoethanol (0.0151 mL), 4-(4,6-dimethoxy-1,3,5-triazin-2-yl)-4-methylmorpholin-4-ium chloride (0.0692 g) and DMF (0.5 mL) was stirred at room temperature for 4 days. The reaction mixture was neutralized with saturated aqueous sodium hydrogen carbonate solution, and the mixture was extracted with ethyl acetate. The organic layer was washed with saturated brine, dried over anhydrous magnesium sulfate and concentrated. The obtained residue was purified by column chromatography (hexane-ethyl acetate and ethyl acetate-methanol) and recrystallized from ethyl acetate-hexane to give the title compound (0.037 g) as a white solid.

¹H-NMR (CDCl₃) δ:2.19 (3H, s), 2.35 (3H, s), 2.62 (1H, brs), 3.58-3.68 (2H, m), 3.85 (2H, s), 3.95 (2H, d), 6.60 (1H, brs), 7.21 (2H, d), 7.72 (2H, d), 7.77-7.83 (1H, m), 7.88-7.95 (1H, m), 8.02 (1H, d).

### Example 85

### 4-{[1-(3-chloro-4-cyanophenyl)-3,5-dimethyl-1H-pyrazol-4-yl]methyl}-N,N-dimethylbenzamide

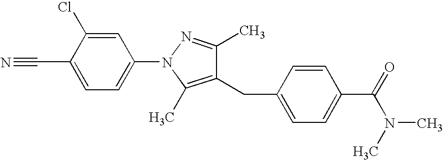

Using 4-{[1-(3-chloro-4-cyanophenyl)-3,5-dimethyl-1H-pyrazol-4-yl]methyl}benzoic acid synthesized in Example 92 and 2 mol/l dimethylamine-THF solution as starting materials and in the same manner as in Example 83, the title compound was obtained.

¹H-NMR (CDCl₃) δ:2.21 (3H, s), 2.34 (3H, s), 3.00 (3H, s), 3.12 (3H, s), 3.83 (2H, s), 7.17 (2H, d), 7.37 (2H, d), 7.53 (1H, dd), 7.72-7.80 (2H, m).

### Example 86

### 2-chloro-4-(3,5-dimethyl-4-{4-[(4-methylpiperazin-1-yl)carbonyl]benzyl}-1H-pyrazol-1-yl)benzonitrile

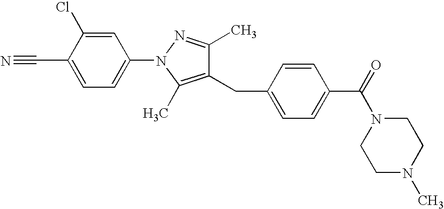

Using 4-{[1-(3-chloro-4-cyanophenyl)-3,5-dimethyl-1H-pyrazol-4-yl]methyl}benzoic acid synthesized in Example 92 and 1-methylpiperazine as starting materials and in the same manner as in Example 83, the title compound was obtained.

¹H-NMR (CDCl₃) δ:2.20 (3H, s), 2.27-2.56 (4H, m), 2.32 (3H, s), 2.33 (3H, s), 3.33-3.58 (2H, m), 3.64-3.91 (2H, m), 3.81 (2H, s), 7.16 (2H, d), 7.34 (2H, d), 7.52 (1H, dd), 7.70-7.79 (2H, m).

### Example 87

### 4-{[1-(3-chloro-4-cyanophenyl)-3,5-dimethyl-1H-pyrazol-4-yl]methyl}-N-methylbenzamide

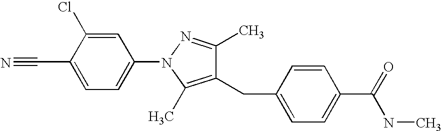

Using 4-{[1-(3-chloro-4-cyanophenyl)-3,5-dimethyl-1H-pyrazol-4-yl]methyl}benzoic acid synthesized in Example 92 and 2 mol/l methylamine-THF solution as starting materials and in the same manner as in Example 83, the title compound was obtained.

¹H-NMR (CDCl₃) δ:2.17 (3H, s), 2.32 (3H, s), 3.01 (3H, d), 3.83 (2H, s), 6.12 (1H, brs), 7.19 (2H, d), 7.52 (1H, d), 7.65-7.82 (4H, m).

### Example 88

### methyl 2-{[1-(3-chloro-4-cyanophenyl)-3,5-dimethyl-1H-pyrazol-4-yl]methyl}benzoate

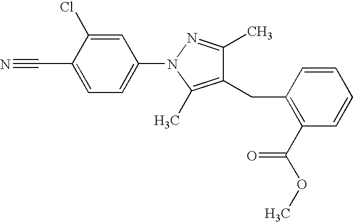

Using methyl 2-[(3,5-dimethyl-1H-pyrazol-4-yl)methyl]benzoate synthesized in Reference Example 35 and 2-chloro-4-fluorobenzonitrile as starting materials and in the same manner as in Example 46, the title compound was obtained.

¹H-NMR (CDCl₃) δ:2.12 (3H, s), 2.26 (3H, s), 3.91 (3H, s), 4.20 (2H, s), 7.01 (1H, d), 7.30 (1H, d), 7.36-7.44 (1H, m), 7.53 (1H, dd), 7.70-7.80 (2H, m), 7.90-7.96 (1H, m)

### Example 89

### methyl 3-{[1-(3-chloro-4-cyanophenyl)-3,5-dimethyl-1H-pyrazol-4-yl]methyl}benzoate

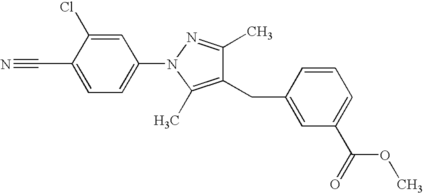

Using methyl 3-[(3,5-dimethyl-1H-pyrazol-4-yl)methyl]benzoate synthesized in Reference Example 36 and 2-chloro-4-fluorobenzonitrile as starting materials and in the same manner as in Example 46, the title compound was obtained.

¹H-NMR (CDCl₃) δ:2.18 (3H, s), 2.34 (3H, s), 3.84 (2H, s), 3.91 (3H, s), 7.28-7.41 (2H, m), 7.52 (1H, dd), 7.70-7.77 (2H, m), 7.84 (1H, s), 7.89 (1H, d).

### Example 90

### 2-{[1-(3-chloro-4-cyanophenyl)-3,5-dimethyl-1H-pyrazol-4-yl]methyl}benzoic acid

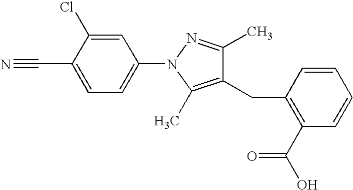

Using methyl 2-{[1-(3-chloro-4-cyanophenyl)-3,5-dimethyl-1H-pyrazol-4-yl]methyl}benzoate synthesized in Example 88 as a starting material and in the same manner as in Example 81, the title compound was obtained.

¹H-NMR (DMSO-d₆) δ:2.00 (3H, s), 2.30 (3H, s), 4.15 (2H, s), 7.06 (1H, d), 7.31 (1H, t), 7.40-7.48 (1H, m), 7.74 (1H, dd), 7.78-7.85 (1H, m), 7.94 (1H, d), 8.08 (1H, d), 13.01 (1H, brs).

### Example 91

### 3-{[1-(3-chloro-4-cyanophenyl)-3,5-dimethyl-1H-pyrazol-4-yl]methyl}benzoic acid

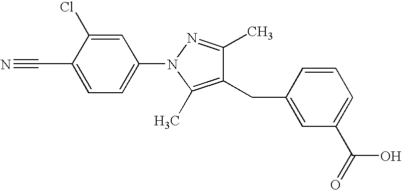

Methyl 3-{[1-(3-chloro-4-cyanophenyl)-3,5-dimethyl-1H-pyrazol-4-yl]methyl}benzoate (2.56 g) synthesized in Example 89 was dissolved in a mixed solvent of THF (30 ml)-methanol (30 mL), 1 mol/l aqueous sodium hydroxide solution (30 mL) was added dropwise slowly at 50° C., and the mixture was stirred at 40° C. for 0.5 hr. The reaction mixture was ice-cooled, acidified with 2 mol/l hydrochloric acid and concentrated under reduced pressure to remove the organic solvent. The obtained suspension was extracted twice with ethyl acetate. The organic layers were combined, washed with saturated brine, dried over anhydrous magnesium sulfate and concentrated to give the title compound (2.47 g) as a white solid.

¹H-NMR (DMSO-d₆) δ:2.10 (3H, s), 2.40 (3H, s), 3.87 (2H, s), 7.39-7.46 (2H, m), 7.72-7.82 (3H, m), 7.96 (1H, d), 8.08 (1H, d), 12.94 (1H, brs).

### Example 92

### 4-{[1-(3-chloro-4-cyanophenyl)-3,5-dimethyl-1H-pyrazol-4-yl]methyl}benzoic acid

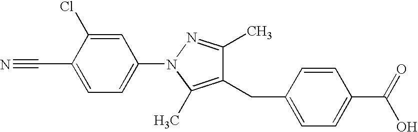

Using methyl 4-{[1-(3-chloro-4-cyanophenyl)-3,5-dimethyl-1H-pyrazol-4-yl]methyl}benzoate synthesized in Example 71 as a starting material and in the same manner as in Example 81, the title compound was obtained.

¹H-NMR (DMSO-d₆) δ:2.10 (3H, s), 2.40 (3H, s), 3.86 (2H, s), 7.29 (2H, d), 7.75 (1H, dd), 7.86 (2H, d), 7.95 (1H, d), 8.08 (1H, d), 12.83 (1H, brs).

### Example 93

### 4-{3,5-dimethyl-4-[(2-methylphenoxy)methyl]-1H-pyrazol-1-yl}benzonitrile

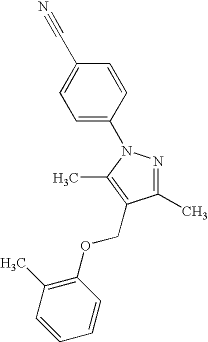

A resin reagent.PS-triphenylphosphine (about 140 mg) was added to Bohdan Inc, a MiniBlock reaction apparatus for preconditioning. Then, a solution (0.150M, 1.00 ml) of o-cresol in THF and a solution (0.200M, 0.500 ml) of 4-[4-(hydroxymethyl)-3,5-dimethyl-1H-pyrazol-1-yl]benzonitrile synthesized in Reference Example 26 in THF were added, and the mixture was stirred. A solution (0.500M, 0.500 ml) of di-tert-butyl azodicarboxylate in THF was added, and the mixture was stirred at room temperature for about 5 days. The reaction mixture was filtered and washed to remove the resin reagent and the filtrate was concentrated. The residue was purified by reversed-phase preparative HPLC (Gilson, Inc. UniPoint system, YMC ODS column 30×75 millimeter, 0.1% TFA containing acetonitrile-water [5:95-100:0]) to give the title compound (0.4 mg).

MS (ESI+, m/e) 318(M+1)

### Example 94

### 4-{3,5-dimethyl-4-[(4-methylphenoxy)methyl]-1H-pyrazol-1-yl}benzonitrile

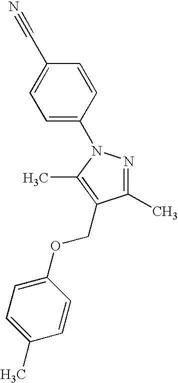

Using p-cresol as a starting material and in the same manner as in Example 93, the title compound was obtained.

MS (ESI+, m/e) 318(M+1).

### Example 95

### 4-{4-[(4-cyanophenoxy)methyl]-3,5-dimethyl-1H-pyrazol-1-yl}benzonitrile

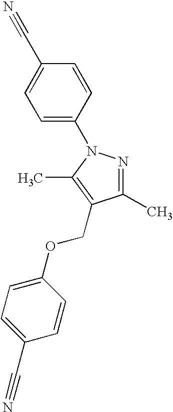

Using 4-hydroxybenzonitrile as a starting material and in the same manner as in Example 93, the title compound was obtained.

MS (ESI+, m/e) 329(M+1).

### Example 96

### 4-{4-[(4-fluoro-2-methylphenoxy)methyl]-3,5-dimethyl-1H-pyrazol-1-yl}benzonitrile

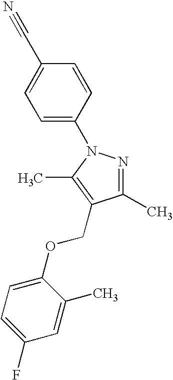

Using 4-fluoro-2-methylphenol as a starting material and in the same manner as in Example 93, the title compound was obtained.

MS (ESI+, m/e) 336(M+1).

### Example 97

### 4-{4-[(4-fluoro-3-methylphenoxy)methyl]-3,5-dimethyl-1H-pyrazol-1-yl}benzonitrile

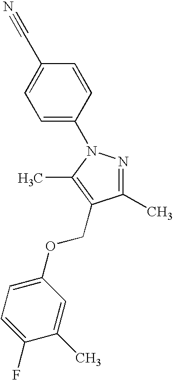

Using 4-fluoro-3-methylphenol as a starting material and in the same manner as in Example 93, the title compound was obtained.

MS (ESI+, m/e) 336(M+1).

### Example 98

### 4-{4-[(2-chlorophenoxy)methyl]-3,5-dimethyl-1H-pyrazol-1-yl}benzonitrile

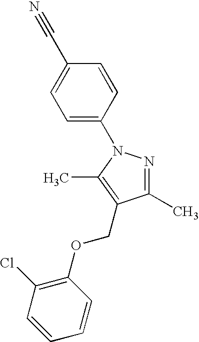

Using 2-chlorophenol as a starting material and in the same manner as in Example 93, the title compound was obtained.

MS (ESI+, m/e) 338(M+1).

### Example 99

### 4-{4-[(4-chlorophenoxy)methyl]-3,5-dimethyl-1H-pyrazol-1-yl}benzonitrile

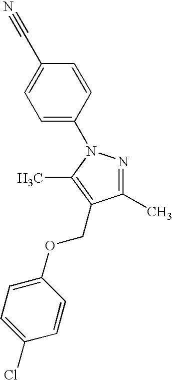

Using 4-chlorophenol as a starting material and in the same manner as in Example 93, the title compound was obtained.

MS (ESI+, m/e) 338(M+1).

### Example 100

### 4-{4-[(2,4-difluorophenoxy)methyl]-3,5-dimethyl-1H-pyrazol-1-yl}benzonitrile

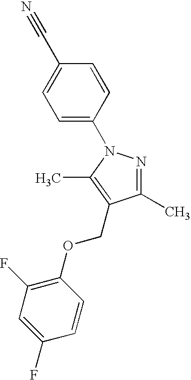

Using 2,4-difluorophenol as a starting material and in the same manner as in Example 93, the title compound was obtained.

MS (ESI+, m/e) 340(M+1).

### Example 101

### 4-{4-[(3,4-difluorophenoxy)methyl]-3,5-dimethyl-1H-pyrazol-1-yl}benzonitrile

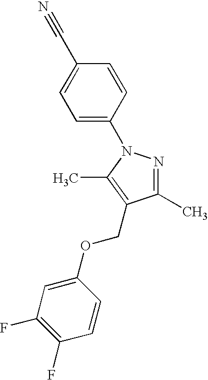

Using 3,4-difluorophenol as a starting material and in the same manner as in Example 93, the title compound was obtained.

MS (ESI+, m/e) 340(M+1).

### Example 102

### 4-{4-[(2,5-difluorophenoxy)methyl]-3,5-dimethyl-1H-pyrazol-1-yl}benzonitrile

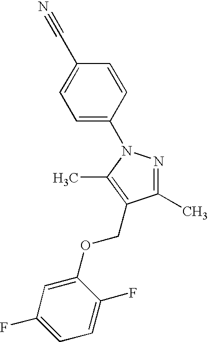

Using 2,5-difluorophenol as a starting material and in the same manner as in Example 93, the title compound was obtained.

MS (ESI+, m/e) 340(M+1).

### Example 103

### 4-{4-[(1H-indol-4-yloxy)methyl]-3,5-dimethyl-1H-pyrazol-1-yl}benzonitrile

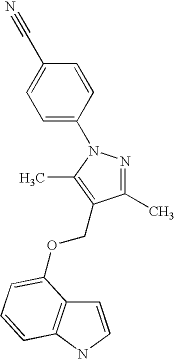

Using 4-hydroxyindole as a starting material and in the same manner as in Example 93, the title compound was obtained.

MS (ESI+, m/e) 343(M+1).

### Example 104

### 4-{4-[(2-acetylphenoxy)methyl]-3,5-dimethyl-1H-pyrazol-1-yl}benzonitrile

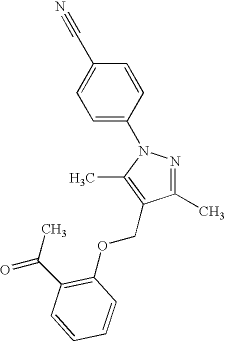

Using 2′-hydroxyacetophenone as a starting material and in the same manner as in Example 93, the title compound was obtained.

MS (ESI+, m/e) 346(M+1).

### Example 105

### 4-{4-[(4-acetylphenoxy)methyl]-3,5-dimethyl-1H-pyrazol-1-yl}benzonitrile

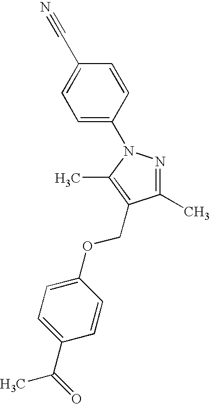

Using 4′-hydroxyacetophenone as a starting material and in the same manner as in Example 93, the title compound was obtained.

MS (ESI+, m/e) 346(M+1).

### Example 106

### 4-(4-{[3-(dimethylamino)phenoxy]methyl}-3,5-dimethyl-1H-pyrazol-1-yl)benzonitrile

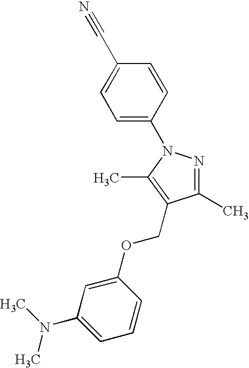

Using 3-dimethylaminophenol as a starting material and in the same manner as in Example 93, the title compound was obtained.

MS (ESI+, m/e) 347(M+1).

### Example 107

### 4-{4-[(1,3-benzodioxol-5-yloxy)methyl]-3,5-dimethyl-1H-pyrazol-1-yl}benzonitrile

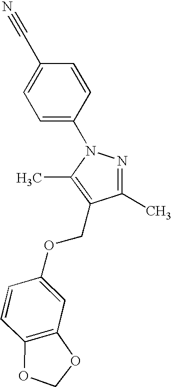

Using sesamol as a starting material and in the same manner as in Example 93, the title compound was obtained.

MS (ESI+, m/e) 348(M+1).

### Example 108

### 4-{3,5-dimethyl-4-[(3-nitrophenoxy)methyl]-1H-pyrazol-1-yl}benzonitrile

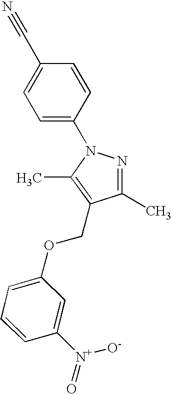

Using 3-nitrophenol as a starting material and in the same manner as in Example 93, the title compound was obtained.

MS (ESI+, m/e) 349(M+1).

### Example 109

### 4-{3,5-dimethyl-4-[(4-nitrophenoxy)methyl]-1H-pyrazol-1-yl}benzonitrile

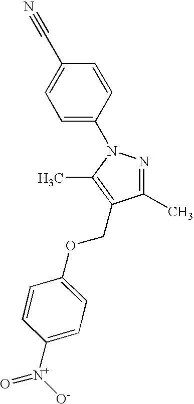

Using 4-nitrophenol as a starting material and in the same manner as in Example 93, the title compound was obtained.

MS (ESI+, m/e) 349(M+1).

### Example 110

### 4-{4-[(4-fluoro-2-methoxyphenoxy)methyl]-3,5-dimethyl-1H-pyrazol-1-yl}benzonitrile

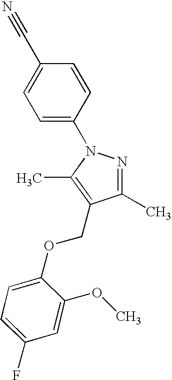

Using 4-fluoro-2-methoxyphenol as a starting material and in the same manner as in Example 93, the title compound was obtained.

MS (ESI+, m/e) 352(M+1).

### Example 111

### 4-{4-[(4-chloro-2-methylphenoxy)methyl]-3,5-dimethyl-1H-pyrazol-1-yl}benzonitrile

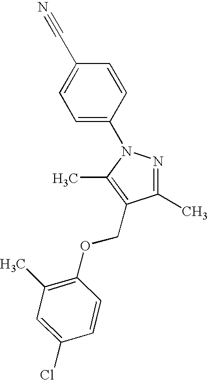

Using 4-chloro-2-methylphenol as a starting material and in the same manner as in Example 93, the title compound was obtained.

MS (ESI+, m/e) 352(M+1).

### Example 112

### 4-{4-[(2-amino-4-chlorophenoxy)methyl]-3,5-dimethyl-1H-pyrazol-1-yl}benzonitrile

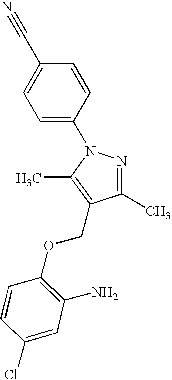

Using 2-amino-4-chlorophenol as a starting material and in the same manner as in Example 93, the title compound was obtained.

MS (ESI+, m/e) 353(M+1).

### Example 113

### 4-{4-[(2-chloro-4-fluorophenoxy)methyl]-3,5-dimethyl-1H-pyrazol-1-yl}benzonitrile

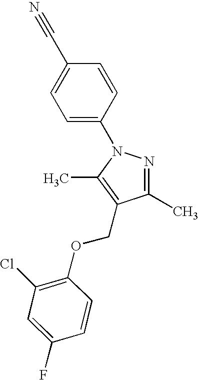

Using 2-chloro-4-fluorophenol as a starting material and in the same manner as in Example 93, the title compound was obtained.

MS (ESI+, m/e) 356(M+1).

### Example 114

### 4-{3,5-dimethyl-4-[(2,4,5-trifluorophenoxy)methyl]-1H-pyrazol-1-yl}benzonitrile

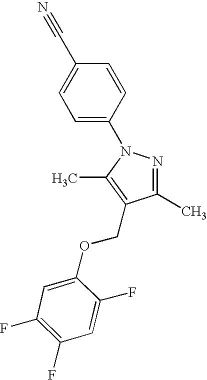

Using 2,4,5-trifluorophenol as a starting material and in the same manner as in Example 93, the title compound was obtained.

MS (ESI+, m/e) 358(M+1).

### Example 115

### 4-{3,5-dimethyl-4-[(2,4,6-trifluorophenoxy)methyl]-1H-pyrazol-1-yl}benzonitrile

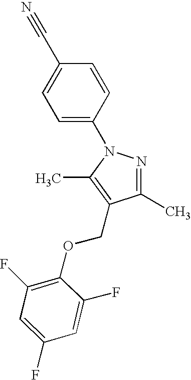

Using 2,4,6-trifluorophenol as a starting material and in the same manner as in Example 93, the title compound was 5 obtained.

MS (ESI+, m/e) 358(M+1).

### Example 116

### 4-(3,5-dimethyl-4-{[(1-oxo-2,3-dihydro-1H-inden-4-yl)oxy]methyl}-1H-pyrazol-1-yl)benzonitrile

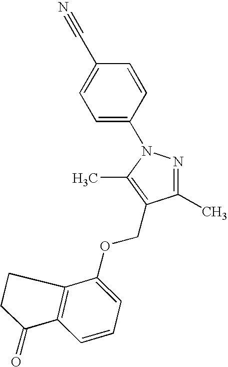

Using 4-hydroxy-1-indanone as a starting material and in the same manner as in Example 93, the title compound was obtained.

MS (ESI+, m/e) 358(M+1).

### Example 117

### 4-{3,5-dimethyl-4-[(5,6,7,8-tetrahydronaphthalen-2-yloxy)methyl]-1H-pyrazol-1-yl}benzonitrile

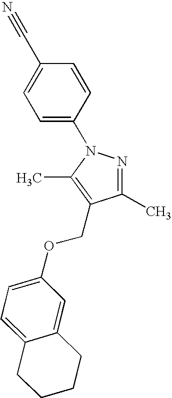

Using 5,6,7,8-tetrahydro-2-naphthol as a starting material and in the same manner as in Example 93, the title compound was obtained.

MS (ESI+, m/e) 358(M+1).

### Example 118

### 4-(3,5-dimethyl-4-{[(3,5,6-trifluoropyridin-2-yl)oxy]methyl}-1H-pyrazol-1-yl)benzonitrile

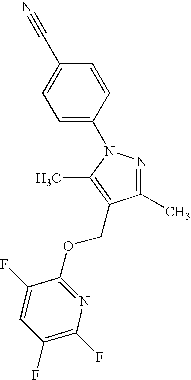

Using 2-hydroxy-3,5,6-trifluoropyridine as a starting material and in the same manner as in Example 93, the title compound was obtained.

MS (ESI+, m/e) 359(M+1).

¹H-NMR (DMSO-d₆) δ:2.28 (3H, s), 2.44 (3H, s), 5.27 (2H, s), 7.76 (2H, d), 7.99 (2H, d), 8.29-8.35 (1H, m).

### Example 119

### 2-(4-{[1-(4-cyanophenyl)-3,5-dimethyl-1H-pyrazol-4-yl]methoxy}phenyl)acetamide

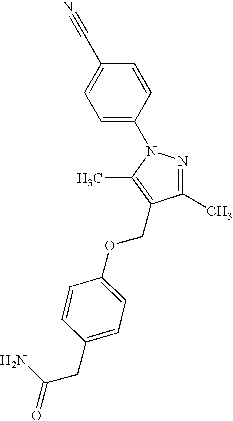

Using 4-hydroxyphenylacetamide as a starting material and in the same manner as in Example 93, the title compound was obtained.

MS (ESI+, m/e) 361(M+1).

### Example 120

### 4-(3,5-dimethyl-4-{[3-methyl-4-(methylsulfanyl)phenoxy]methyl}-1H-pyrazol-1-yl)benzonitrile

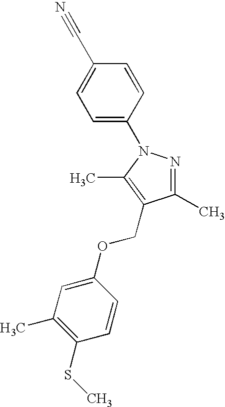

Using 3-methyl-4-(methylthio)phenol as a starting material and in the same manner as in Example 93, the title compound was obtained.

MS (ESI+, m/e) 364(M+1).

### Example 121

### 4-(3,5-dimethyl-4-{[4-(1H-pyrazol-1-yl)phenoxy]methyl}-1H-pyrazol-1-yl)benzonitrile

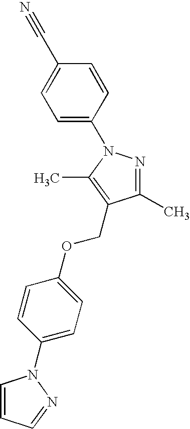

Using 4-(1H-pyrazol-1-yl)phenol as a starting material and in the same manner as in Example 93, the title compound was obtained.

MS (ESI+, m/e) 370(M+1).

### Example 122

### 4-(3,5-dimethyl-4-{[4-(1H-1,2,4-triazol-1-yl)phenoxy]methyl}-1H-pyrazol-1-yl)benzonitrile

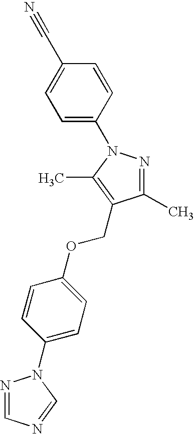

Using 4-(1,2,4-triazol-1-yl)phenol as a starting material and in the same manner as in Example 93, the title compound was obtained.

MS (ESI+, m/e) 371 (M+1)

### Example 123

### 4-(3,5-dimethyl-4-{[4-(1,3,4-oxadiazol-2-yl)phenoxy]methyl}-1H-pyrazol-1-yl)benzonitrile

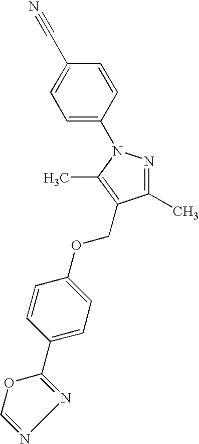

Using 4-(1,3,4-oxadiazol-2-yl)phenol as a starting material and in the same manner as in Example 93, the title compound was obtained.

MS (ESI+, m/e) 372(M+1).

### Example 124

### 4-(3,5-dimethyl-4-{[4-(trifluoromethyl)phenoxy]methyl}-1H-pyrazol-1-yl)benzonitrile

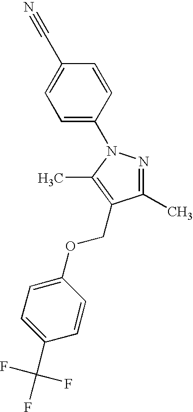

Using 4-(trifluoromethyl)phenol as a starting material and in the same manner as in Example 93, the title compound was obtained.

MS (ESI+, m/e) 372(M+1).

### Example 125

### 4-{4-[(2,3-dichlorophenoxy)methyl]-3,5-dimethyl-1H-pyrazol-1-yl}benzonitrile

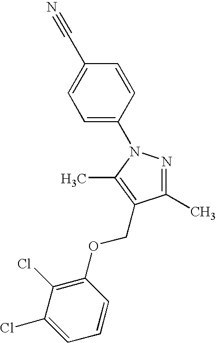

Using 2,3-dichlorophenol as a starting material and in the same manner as in Example 93, the title compound was obtained.

MS (ESI+, m/e) 372(M+1).

### Example 126

### 4-{4-[(3,5-dichlorophenoxy)methyl]-3,5-dimethyl-1H-pyrazol-1-yl}benzonitrile

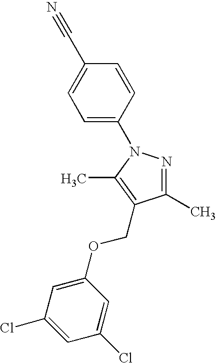

Using 3,5-dichlorophenol as a starting material and in the same manner as in Example 93, the title compound was obtained.

MS (ESI+, m/e) 372(M+1).

### Example 127

### 4-(4-{[(2,2-dimethyl-2,3-dihydro-1-benzofuran-7-yl)oxy]methyl}-3,5-dimethyl-1H-pyrazol-1-yl)benzonitrile

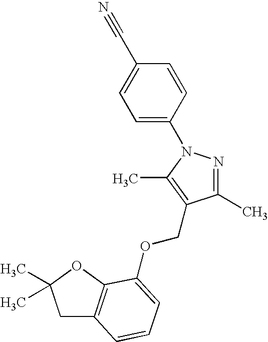

Using 2,3-dihydro-2,2-dimethyl-7-hydroxybenzofuran as a starting material and in the same manner as in Example 93, the title compound was obtained.

MS (ESI+, m/e) 374(M+1).

### Example 128

### 4-{4-[(3-chloro-4-fluorophenoxy)methyl]-3,5-dimethyl-1H-pyrazol-1-yl}benzonitrile

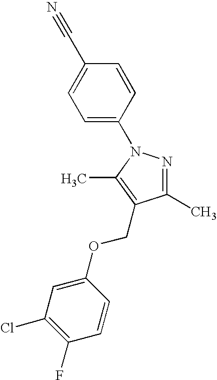

Using 3-chloro-4-fluorophenol as a starting material and in the same manner as in Example 93, the title compound was obtained.

MS (ESI+, m/e) 356(M+1).

¹H-NMR (DMSO-d₆) δ:2.23 (3H, s), 2.40 (3H, s), 4.97 (2H, s), 7.03 (1H, dt), 7.31 (1H, dd), 7.36 (1H, t), 7.77 (2H, d), 7.99 (2H, d).

### Example 129

### methyl 2-{[1-(4-cyanophenyl)-3,5-dimethyl-1H-pyrazol-4-yl]methoxy}-5-fluorobenzoate

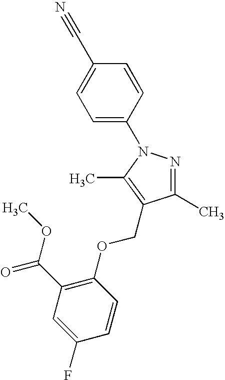

Using 5-fluoro-2-methyl hydroxybenzoate as a starting material and in the same manner as in Example 93, the title compound was obtained.

MS (ESI+, m/e) 380 (M+1).

### Example 130

### 4-{4-[(4-bromophenoxy)methyl]-3,5-dimethyl-1H-pyrazol-1-yl}benzonitrile

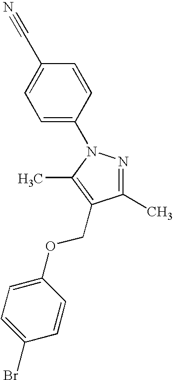

Using 4-bromophenol as a starting material and in the same manner as in Example 93, the title compound was obtained.

MS (ESI+, m/e) 382(M+1).

### Example 131

### 4-{4-[(4-cyclohexylphenoxy)methyl]-3,5-dimethyl-1H-pyrazol-1-yl}benzonitrile

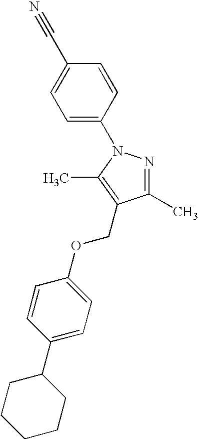

Using 4-cyclohexylphenol as a starting material and in the same manner as in Example 93, the title compound was obtained.

MS (ESI+, m/e) 386(M+1)

### Example 132

### 4-(3,5-dimethyl-4-{[4-(2-oxopyrrolidin-1-yl)phenoxy]methyl}-1H-pyrazol-1-yl)benzonitrile

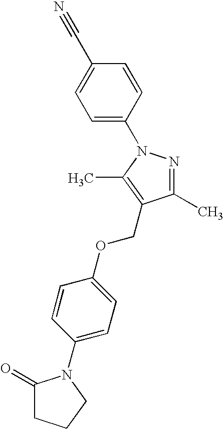

Using 1-(4-hydroxyphenyl)pyrrolidin-2-one as a starting material and in the same manner as in Example 93, the title compound was obtained.

MS (ESI+, m/e) 387(M+1).

### Example 133

### 4-(3,5-dimethyl-4-{[4-(trifluoromethoxy)phenoxy]methyl}-1H-pyrazol-1-yl)benzonitrile

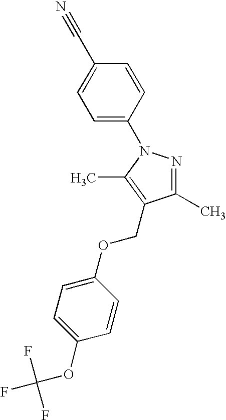

Using 4-(trifluoromethoxy)phenol as a starting material and in the same manner as in Example 93, the title compound was obtained.

MS (ESI+, m/e) 388(M+1).

### Example 134

### 4-(4-{[4-fluoro-2-(1H-pyrazol-3-yl)phenoxy]methyl}-3,5-dimethyl-1H-pyrazol-1-yl)benzonitrile

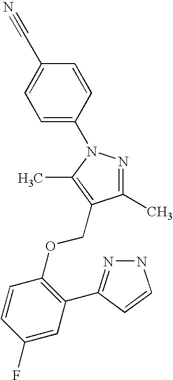

Using 3-(3-fluoro-6-hydroxyphenyl)pyrazole as a starting material and in the same manner as in Example 93, the title compound was obtained.

MS (ESI+, m/e) 388(M+1).

### Example 135

### 4-{4-[(4-fluoro-2-isoxazol-5-ylphenoxy)methyl]-3,5-dimethyl-1H-pyrazol-1-yl}benzonitrile

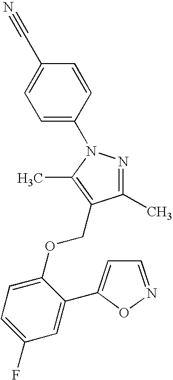

Using 5-(3-fluoro-6-hydroxyphenyl)isoxazole as a starting material and in the same manner as in Example 93, the title compound was obtained.

MS (ESI+, m/e) 389(M+1).

### Example 136

### 4-{3,5-dimethyl-4-[(3-morpholin-4-ylphenoxy)methyl]-1H-pyrazol-1-yl}benzonitrile

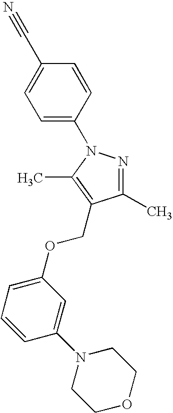

Using 3-morpholinophenol as a starting material and in the same manner as in Example 93, the title compound was obtained.

MS (ESI+, m/e) 389(M+1).

### Example 137

### 4-(4-{[4-fluoro-3-(trifluoromethoxy)phenoxy]methyl}-3,5-dimethyl-1H-pyrazol-1-yl)benzonitrile

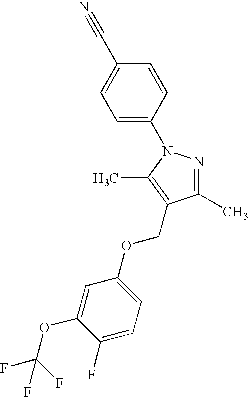

Using 4-fluoro-3-(trifluoromethoxy)phenol as a starting material and in the same manner as in Example 93, the title compound was obtained.

MS (ESI+, m/e) 406(M+1).

### Example 138

### 4-(4-{[4-fluoro-3-(trifluoromethyl)phenoxy]methyl}-3,5-dimethyl-1H-pyrazol-1-yl)benzonitrile

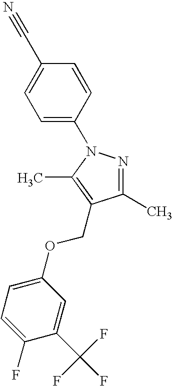

Using 4-fluoro-3-(trifluoromethyl)phenol as a starting material and in the same manner as in Example 93, the title compound was obtained.

MS (ESI+, m/e) 390(M+1).

¹H-NMR (DMSO-d₆) δ:2.24 (3H, s), 2.41 (3H, s), 5.04 (2H, s), 7.36-7.40 (2H, m), 7.48 (1H, t), 7.77 (2H, d), 7.99 (2H, d).

### Example 139

### 4-{3,5-dimethyl-4-[(4-phenoxyphenoxy)methyl]-1H-pyrazole-1-yl}benzonitrile

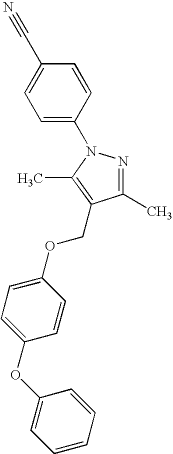

Using 4-phenoxyphenol as a starting material and in the same manner as in Example 93, the title compound was obtained.

MS (ESI+, m/e) 396(M+1).

### Example 140

### 4-[3,5-dimethyl-4-({4-[(trifluoromethyl)sulfanyl]phenoxy}methyl)-1H-pyrazol-1-yl]benzonitrile

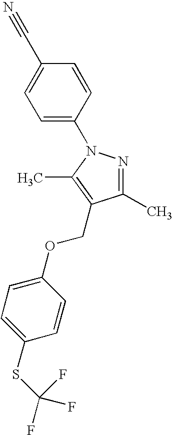

Using 4-(trifluoromethylthio)phenol as a starting material and in the same manner as in Example 93, the title compound was obtained.

MS (ESI+, m/e) 404(M+1).

### Example 141

### 4-(4-{[3,5-bis(trifluoromethyl)phenoxy]methyl}-3,5-dimethyl-1H-pyrazol-1-yl)benzonitrile

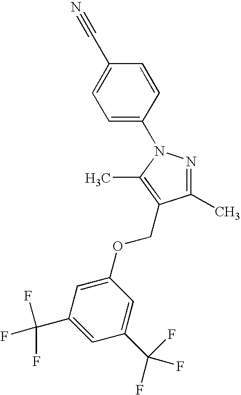

Using 3,5-bis(trifluoromethyl)phenol as a starting material and in the same manner as in Example 93, the title compound was obtained.

MS (ESI+, m/e) 440(M+1).

### Example 142

### 2-chloro-4-[3,5-dimethyl-4-(phenoxymethyl)-1H-pyrazol-1-yl]benzonitrile

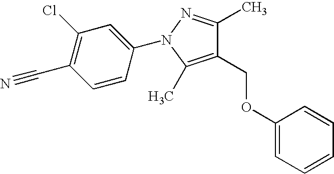

A resin reagent.PS-triphenylphosphine (about 140 mg) was added to Bohdan Inc, a MiniBlock reaction apparatus for preconditioning. Then, a solution (0.150M, 1.00 ml) of phenol in THF and a solution (0.200M, 0.500 ml) of 2-chloro-4-[4-(hydroxymethyl)-3,5-dimethyl-1H-pyrazol-1-yl]benzonitrile synthesized in Reference Example 21 in THF was added, and the mixture was stirred. A solution (0.500M, 0.500 ml) of di-tert-butyl azodicarboxylate in THF was added, and the mixture was stirred at room temperature for about 5 days. The reaction mixture was filtered and washed to remove the resin reagent and the filtrate was concentrated. The residue was purified by reversed-phase preparative HPLC (Gilson, Inc. UniPoint system, YMC ODS column 30×75 millimeter, 0.1% TFA containing acetonitrile-water [5:95-100:0]) to give the title compound (5.8 mg).

MS (ESI+, m/e) 338(M+1)

### Example 143

### 2-chloro-4-{3,5-dimethyl-4-[(2-methylphenoxy)methyl]-1H-pyrazol-1-yl}benzonitrile

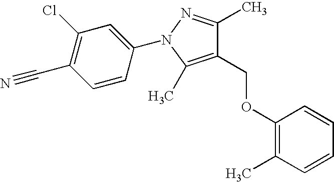

Using o-cresol as a starting material and in the same manner as in Example 142, the title compound was obtained.

MS (ESI+, m/e) 352(M+1).

### Example 144

### 2-chloro-4-{3,5-dimethyl-4-[(4-methylphenoxy)methyl]-1H-pyrazol-1-yl}benzonitrile

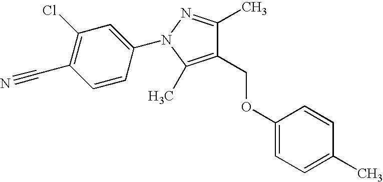

Using p-cresol as a starting material and in the same manner as in Example 142, the title compound was obtained.

MS (ESI+, m/e) 352(M+1).

### Example 145

### 2-chloro-4-(4-{[(5-fluoropyrimidin-2-yl)oxy]methyl}-3,5-dimethyl-1H-pyrazol-1-yl)benzonitrile

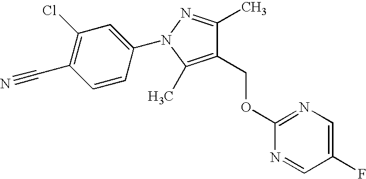

Using 5-fluoro-2-hydroxypyrimidine as a starting material and in the same manner as in Example 142, the title compound was obtained.

MS (ESI+, m/e) 358(M+1).

### Example 146

### 2-chloro-4-{4-[(4-cyanophenoxy)methyl]-3,5-dimethyl-1H-pyrazol-1-yl}benzonitrile

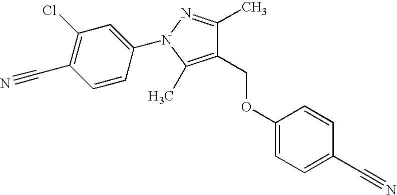

Using 4-hydroxybenzonitrile as a starting material and in the same manner as in Example 142, the title compound was obtained.

MS (ESI+, m/e) 363(M+1).

### Example 147

### 2-chloro-4-{4-[(3-methoxyphenoxy)methyl]-3,5-dimethyl-1H-pyrazol-1-yl}benzonitrile

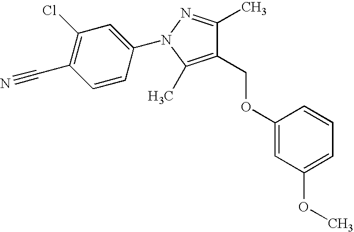

Using 3-methoxyphenol as a starting material and in the same manner as in Example 142, the title compound was obtained.

MS (ESI+, m/e) 368(M+1).

### Example 148

### 2-chloro-4-{4-[(4-methoxyphenoxy)methyl]-3,5-dimethyl-1H-pyrazol-1-yl}benzonitrile

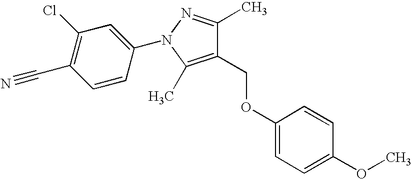

Using 4-methoxyphenol as a starting material and in the same manner as in Example 142, the title compound was obtained.

MS (ESI+, m/e) 368(M+1).

### Example 149

### 2-chloro-4-{4-[(4-fluoro-2-methylphenoxy)methyl]-3,5-dimethyl-1H-pyrazol-1-yl}benzonitrile

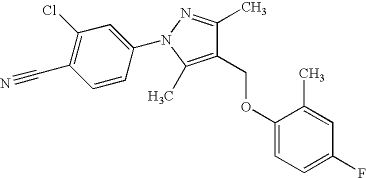

Using 4-fluoro-2-methylphenol as a starting material and in the same manner as in Example 142, the title compound was obtained.

MS (ESI+, m/e) 370(M+1).

### Example 150

### 2-chloro-4-{4-[(4-fluoro-3-methylphenoxy)methyl]-3,5-dimethyl-1H-pyrazol-1-yl}benzonitrile

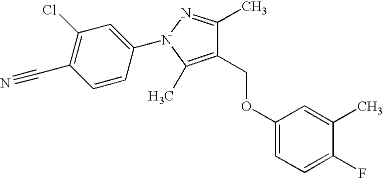

Using 4-fluoro-3-methylphenol as a starting material and in the same manner as in Example 142, the title compound was obtained.

MS (ESI+, m/e) 370(M+1).

### Example 151

### 2-chloro-4-{4-[(2-chlorophenoxy)methyl]-3,5-dimethyl-1H-pyrazol-1-yl}benzonitrile

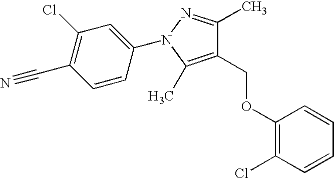

Using 2-chlorophenol as a starting material and in the same manner as in Example 142, the title compound was obtained.

MS (ESI+, m/e) 372(M+1).

### Example 152

### 2-chloro-4-{4-[(4-chlorophenoxy)methyl]-3,5-dimethyl-1H-pyrazol-1-yl}benzonitrile

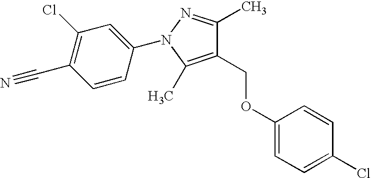

Using 4-chlorophenol as a starting material and in the same manner as in Example 142, the title compound was obtained.

MS (ESI+, m/e) 372(M+1).

### Example 153

### 2-chloro-4-{4-[(2,4-difluorophenoxy)methyl]-3,5-dimethyl-1H-pyrazol-1-yl}benzonitrile

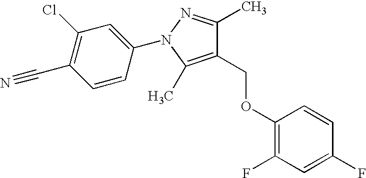

Using 2,4-difluorophenol as a starting material and in the same manner as in Example 142, the title compound was obtained.

MS (ESI+, m/e) 374 (M+1)

### Example 154

### 2-chloro-4-{4-[(3,4-difluorophenoxy)methyl]-3,5-dimethyl-1H-pyrazol-1-yl}benzonitrile

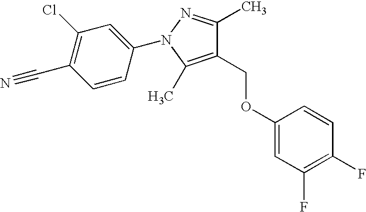

Using 3,4-difluorophenol as a starting material and in the same manner as in Example 142, the title compound was obtained.

MS (ESI+, m/e) 374(M+1).

### Example 155

### 2-chloro-4-{4-[(2,5-difluorophenoxy)methyl]-3,5-dimethyl-1H-pyrazol-1-yl}benzonitrile

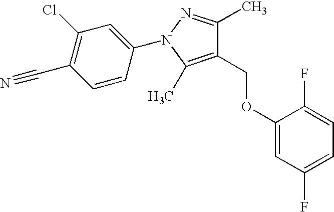

Using 2,5-difluorophenol as a starting material and in the same manner as in Example 142, the title compound was obtained.

MS (ESI+, m/e) 374(M+1).

### Example 156

### 2-chloro-4-{4-[(1H-indol-4-yloxy)methyl]-3,5-dimethyl-1H-pyrazol-1-yl}benzonitrile

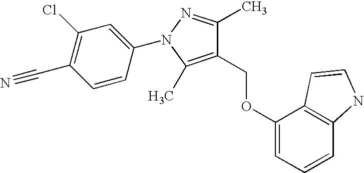

Using 4-hydroxyindole as a starting material and in the same manner as in Example 142, the title compound was obtained.

MS (ESI+, m/e) 377(M+1).

### Example 157

### 2-chloro-4-(4-{[4-(cyanomethyl)phenoxy]methyl}-3,5-dimethyl-1H-pyrazol-1-yl)benzonitrile

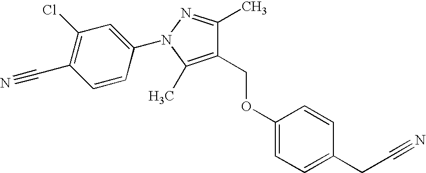

Using 4-hydroxyphenylacetonitrile as a starting material and in the same manner as in Example 142, the title compound was obtained.

MS (ESI+, m/e) 377(M+1).

### Example 158

### 4-{4-[(2-acetylphenoxy)methyl]-3,5-dimethyl-1H-pyrazol-1-yl}-2-chlorobenzonitrile

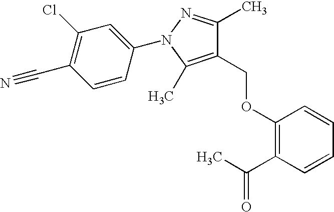

Using 2′-hydroxyacetophenone as a starting material and in the same manner as in Example 142, the title compound was obtained.

MS (ESI+, m/e) 380(M+1).

### Example 159

### 4-{4-[(4-acetylphenoxy)methyl]-3,5-dimethyl-1H-pyrazol-1-yl}-2-chlorobenzonitrile

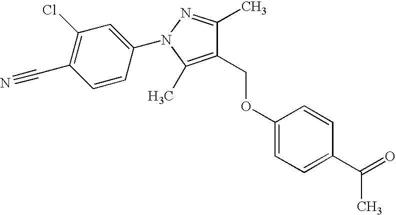

Using 4′-hydroxyacetophenone as a starting material and in the same manner as in Example 142, the title compound was obtained.

MS (ESI+, m/e) (M+1).

### Example 160

### 2-chloro-4-(4-{[3-(dimethylamino)phenoxy]methyl}-3,5-dimethyl-1H-pyrazol-1-yl)benzonitrile

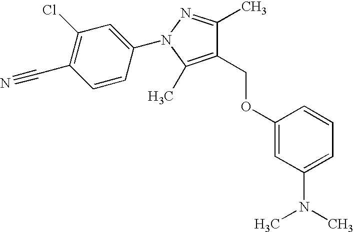

Using 3-dimethylaminophenol as a starting material and in the same manner as in Example 142, the title compound was obtained.

MS (ESI+, m/e) 381(M+1).

### Example 161

### 4-{4-[(1,3-benzodioxol-5-yloxy)methyl]-3,5-dimethyl-1H-pyrazol-1-yl}-2-chlorobenzonitrile

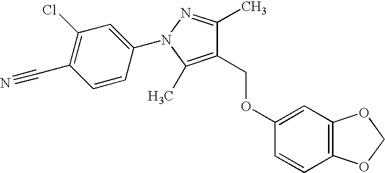

Using sesamol as a starting material and in the same manner as in Example 142, the title compound was obtained.

MS (ESI+, m/e) 382(M+1).

### Example 162

### 2-chloro-4-{4-[(2-methoxy-4-methylphenoxy)methyl]-3,5-dimethyl-1H-pyrazol-1-yl}benzonitrile

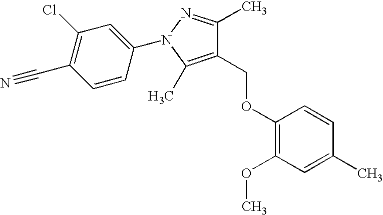

Using 2-methoxy-4-methylphenol as a starting material and in the same manner as in Example 142, the title compound was obtained.

MS (ESI+, m/e) 382(M+1).

### Example 163

### 2-chloro-4-{3,5-dimethyl-4-[(3-nitrophenoxy)methyl]-1H-pyrazol-1-yl}benzonitrile

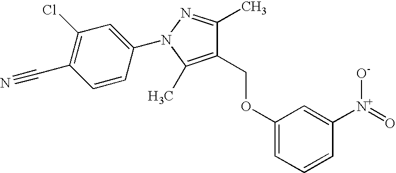

Using 3-nitrophenol as a starting material and in the same manner as in Example 142, the title compound was obtained.

MS (ESI+, m/e) 383(M+1).

### Example 164

### 2-chloro-4-{4-[(4-fluoro-2-methoxyphenoxy)methyl]-3,5-dimethyl-1H-pyrazol-1-yl}benzonitrile

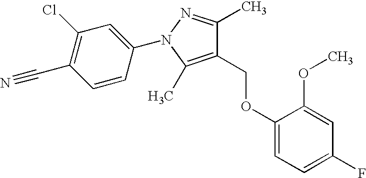

Using 4-fluoro-2-methoxyphenol as a starting material and in the same manner as in Example 142, the title compound was obtained.

MS (ESI+, m/e) 386(M+1).

### Example 165

### 2-chloro-4-{4-[(4-chloro-2-methylphenoxy)methyl]-3,5-dimethyl-1H-pyrazol-1-yl}benzonitrile

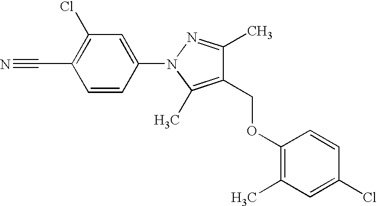

Using 4-chloro-2-methylphenol as a starting material and in the same manner as in Example 142, the title compound was obtained.

MS (ESI+, m/e) 386(M+1).

### Example 166

### 4-{4-[(2-amino-4-chlorophenoxy)methyl]-3,5-dimethyl-1H-pyrazol-1-yl}-2-chlorobenzonitrile

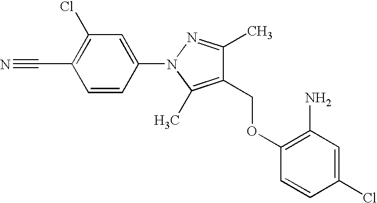

Using 2-amino-4-chlorophenol as a starting material and in the same manner as in Example 142, the title compound was obtained.

MS (ESI+, m/e) 387(M+1).

### Example 167

### 2-chloro-4-{4-[(2-chloro-4-fluorophenoxy)methyl]-3,5-dimethyl-1H-pyrazol-1-yl}benzonitrile

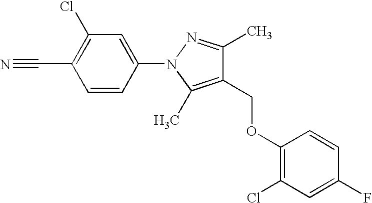

Using 2-chloro-4-fluorophenol as a starting material and in the same manner as in Example 142, the title compound was obtained.

MS (ESI+, m/e) 390(M+1).

### Example 168

### 2-chloro-4-{3,5-dimethyl-4-[(2,3,4-trifluorophenoxy)methyl]-1H-pyrazol-1-yl}benzonitrile

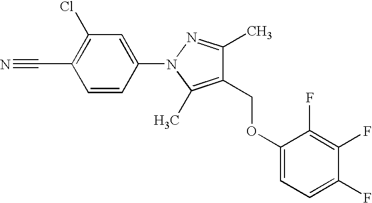

Using 2,3,4-trifluorophenol as a starting material and in the same manner as in Example 142, the title compound was obtained.

MS (ESI+, m/e) 392(M+1).

### Example 169

### 2-chloro-4-{3,5-dimethyl-4-[(2,4,5-trifluorophenoxy)methyl]-1H-pyrazol-1-yl}benzonitrile

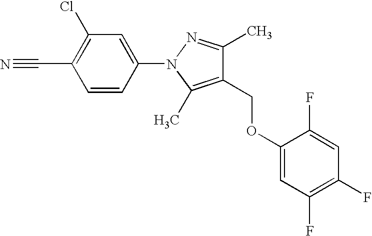

Using 2,4,5-trifluorophenol as a starting material and in the same manner as in Example 142, the title compound was obtained.

MS (ESI+, m/e) 392(M+1).

### Example 170

### 2-chloro-4-{3,5-dimethyl-4-[(2,4,6-trifluorophenoxy)methyl]-1H-pyrazol-1-yl}benzonitrile

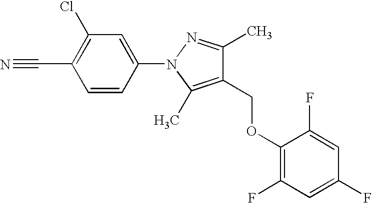

Using 2,4,6-trifluorophenol as a starting material and in the same manner as in Example 142, the title compound was obtained.

MS (ESI+, m/e) 392(M+1).

### Example 171

### 2-chloro-4-{3,5-dimethyl-4-[(3,4,5-trifluorophenoxy)methyl]-1H-pyrazol-1-yl}benzonitrile

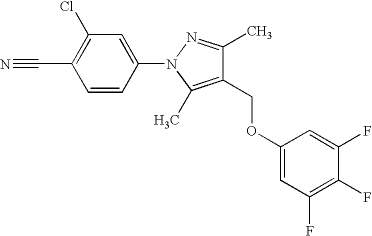

Using 3,4,5-trifluorophenol as a starting material and in the same manner as in Example 142, the title compound was obtained.

MS (ESI+, m/e) 392(M+1).

### Example 172

### 2-chloro-4-(3,5-dimethyl-4-{[(1-oxo-2,3-dihydro-1H-inden-4-yl)oxy]methyl}-1H-pyrazol-1-yl)benzonitrile

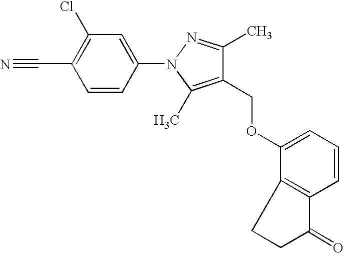

Using 4-hydroxy-1-indanone as a starting material and in the same manner as in Example 142, the title compound was obtained.

MS (ESI+, m/e) 392(M+1).

### Example 173

### 2-chloro-4-{3,5-dimethyl-4-[(5,6,7,8-tetrahydronaphthalen-2-yloxy)methyl]-1H-pyrazol-1-yl}benzonitrile

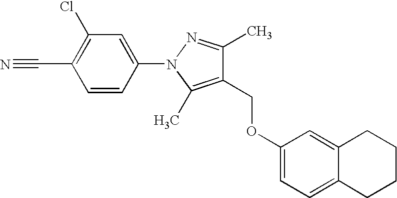

Using 5,6,7,8-tetrahydro-2-naphthol as a starting material and in the same manner as in Example 142, the title compound was obtained.

MS (ESI+, m/e) 392(M+l).

### Example 174

### 2-chloro-4-(3,5-dimethyl-4-{[(3,5,6-trifluoropyridin-2-yl)oxy]methyl}-1H-pyrazol-1-yl)benzonitrile

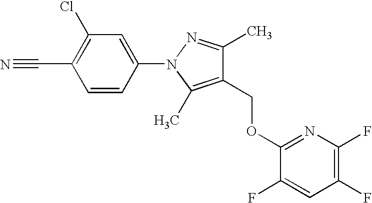

Using 2-hydroxy-3,5,6-trifluoropyridine as a starting material and in the same manner as in Example 142, the title compound was obtained.

MS (ESI+, m/e) 393(M+l).

### Example 175

### 2-chloro-4-(3,5-dimethyl-4-{[(2-oxo-2,3-dihydro-1-benzofuran-5-yl)oxy]methyl}-1H-pyrazol-1-yl)benzonitrile

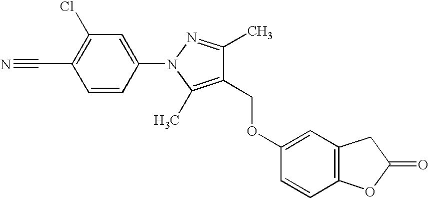

Using homogentisic acid γ-lactone as a starting material and in the same manner as in Example 142, the title compound was obtained.

MS (ESI+, m/e) 394 (M+1).

### Example 176

### 4-{4-[(4-tert-butylphenoxy)methyl]-3,5-dimethyl-1H-pyrazol-1-yl}-2-chlorobenzonitrile

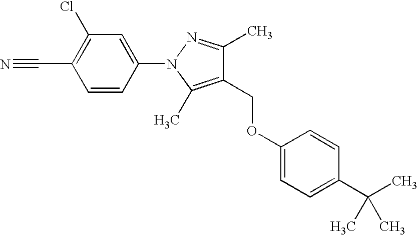

Using 4-tert-butylphenol as a starting material and in the same manner as in Example 142, the title compound was obtained.

MS (ESI+, m/e) 394(M+1).

¹H-NMR (DMSO-d₆) δ:1.26 (9H, s), 2.23 (3H, s), 2.44 (3H, s), 4.90 (2H, s), 6.94 (2H, d), 7.31 (2H, d), 7.77 (1H, dd), 7.97 (1H, d), 8.11 (1H, d).

### Example 177

### N-(4-{[1-(3-chloro-4-cyanophenyl)-3,5-dimethyl-1H-pyrazol-4-yl]methoxy}phenyl)acetamide

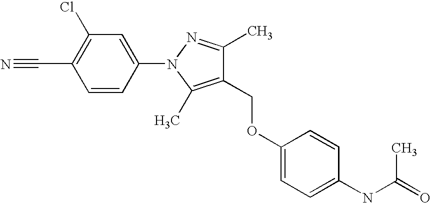

Using acetaminophen as a starting material and in the same manner as in Example 142, the title compound was obtained.

MS (ESI+, m/e) 395(M+1).

### Example 178

### 2-(4-{[1-(3-chloro-4-cyanophenyl)-3,5-dimethyl-1H-pyrazol-4-yl]methoxy}phenyl)acetamide

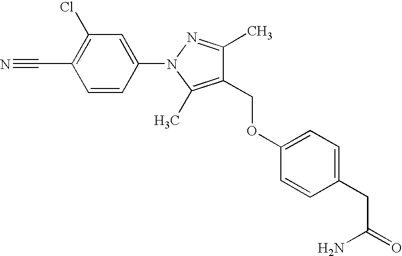

Using 4-hydroxyphenylacetamide as a starting material and in the same manner as in Example 142, the title compound was obtained.

MS (ESI+, m/e) 395(M+l).

### Example 179

### 4-{[1-(3-chloro-4-cyanophenyl)-3,5-dimethyl-1H-pyrazol-4-yl]methoxy}benzenecarbothioamide

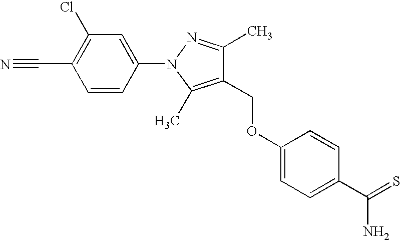

Using 4-hydroxy-thiobenzamide as a starting material and in the same manner as in Example 142, the title compound was obtained.

MS (ESI+, m/e) 397(M+1).

### Example 180

### 2-chloro-4-(3,5-dimethyl-4-{[3-methyl-4-(methylsulfanyl)phenoxy]methyl}-1H-pyrazol-1-yl)benzonitrile

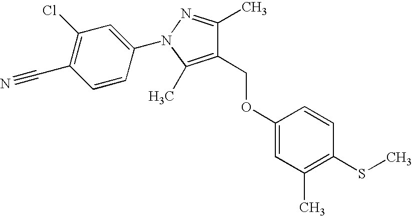

Using 3-methyl-4-(methylthio)phenol as a starting material and in the same manner as in Example 142, the title compound was obtained.

MS (ESI+, m/e) 398(M+1).

### Example 181

### 2-chloro-4-(3,5-dimethyl-4-{[4-(1H-pyrazole-1-yl)phenoxy]methyl}-1H-pyrazol-1-yl)benzonitrile

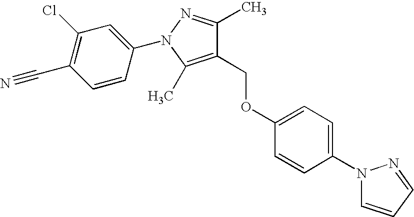

Using 4-(1H-pyrazol-1-yl)phenol as a starting material and in the same manner as in Example 142, the title compound was obtained.

MS (ESI+, m/e) 404(M+1).

### Example 182

### 2-chloro-4-(4-{[4-(1H-imidazol-1-yl)phenoxy]methyl}-3,5-dimethyl-1H-pyrazol-1-yl)benzonitrile

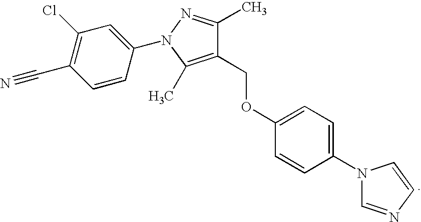

Using 4-(imidazol-1-yl)phenol as a starting material and in the same manner as in Example 142, the title compound was obtained.

MS (ESI+, m/e) 404(M+1).

¹H-NMR (DMSO-d₆) δ:2.26 (3H, s), 2.46 (3H, s), 5.01 (2H, s), 7.08 (1H, s), 7.17 (2H, d), 7.58 (2H, d), 7.66 (1H, s), 7.77 (1H, dd), 7.98 (1H, d), 8.12 (1H, d), 8.15 (1H, s).

### Example 183

### 2-chloro-4-(3,5-dimethyl-4-{[4-(1,3,4-oxadiazol-2-yl)phenoxy]methyl}-1H-pyrazol-1-yl)benzonitrile

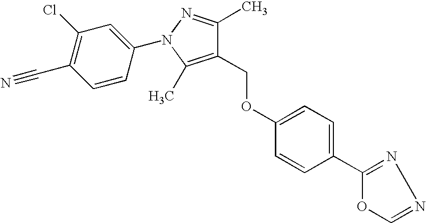

Using 4-(1,3,4-oxadiazol-2-yl)phenol as a starting material and in the same manner as in Example 142, the title compound was obtained.

MS (ESI+, m/e) 406(M+1).

### Example 184

### 2-chloro-4-(3,5-dimethyl-4-{[4-(trifluoromethyl)phenoxy]methyl}-1H-pyrazol-1-yl)benzonitrile

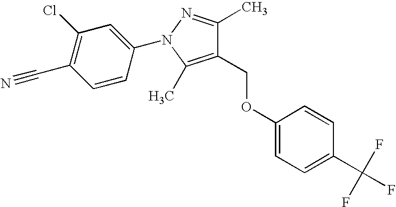

Using 4-(trifluoromethyl)phenol as a starting material and in the same manner as in Example 142, the title compound was obtained.

MS (ESI+, m/e) 406(M+1).

### Example 185

### 2-chloro-4-{4-[(3,5-dichlorophenoxy)methyl]-3,5-dimethyl-1H-pyrazol-1-yl}benzonitrile

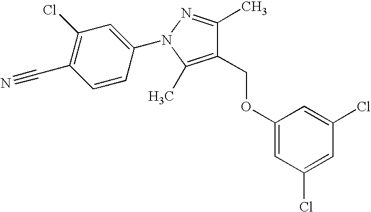

Using 3,5-dichlorophenol as a starting material and in the same manner as in Example 142, the title compound was obtained.

MS (ESI+, m/e) 406(M+1).

### Example 186

### 2-chloro-4-(4-{[(5-fluoroquinolin-8-yl)oxy]methyl}-3,5-dimethyl-1H-pyrazol-1-yl)benzonitrile

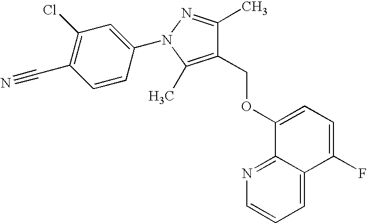

Using 5-fluoro-8-hydroxyquinoline as a starting material and in the same manner as in Example 142, the title compound was obtained.

MS (ESI+, m/e) 407(M+1).

### Example 187

### 2-chloro-4-(4-{[(2,2-dimethyl-2,3-dihydro-1-benzofuran-7-yl)oxy]methyl}-3,5-dimethyl-1H-pyrazol-1-yl)benzonitrile

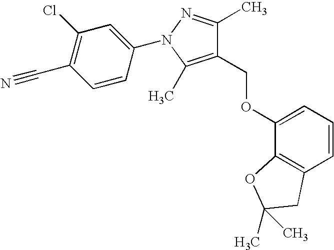

Using 2,3-dihydro-2,2-dimethyl-7-hydroxybenzofuran as a starting material and in the same manner as in Example 142, the title compound was obtained.

MS (ESI+, m/e) 408(M+1).

### Example 188

### 2-chloro-4-{4-[(3-chloro-4-fluorophenoxy)methyl]-3,5-dimethyl-1H-pyrazol-1-yl}benzonitrile

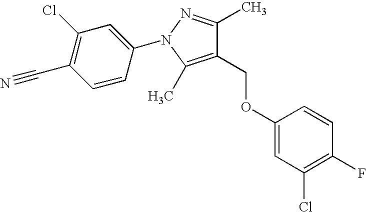

Using 3-chloro-4-fluorophenol as a starting material and in the same manner as in Example 142, the title compound was obtained.

MS (ESI+, m/e) 390(M+1).

### Example 189

### methyl 2-{[1-(3-chloro-4-cyanophenyl)-3,5-dimethyl-1H-pyrazol-4-yl]methoxy}-5-fluorobenzoate

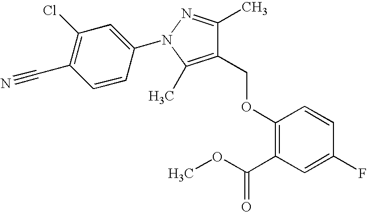

Using methyl 5-fluoro-2-hydroxybenzoate as a starting material and in the same manner as in Example 142, the title compound was obtained.

MS (ESI+, m/e) 414(M+1).

### Example 190

### 2-chloro-4-(3,5-dimethyl-4-{[4-(methylsulfonyl)phenoxy]methyl}-1H-pyrazol-1-yl)benzonitrile

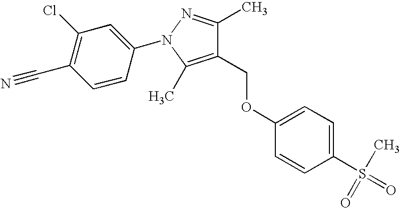

Using 4-methylsulfonylphenol as a starting material and in the same manner as in Example 1.42, the title compound was obtained.

MS (ESI+, m/e) 416(M+1).

### Example 191

### 4-{4-[(3-bromophenoxy)methyl]-3,5-dimethyl-1H-pyrazol-1-yl}-2-chlorobenzonitrile

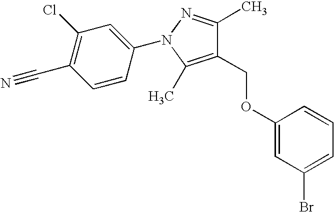

Using 3-bromophenol as a starting material and in the same manner as in Example 142, the title compound was obtained.

MS (ESI+, m/e) 416(M+1).

### Example 192

### 2-chloro-4-(3,5-dimethyl-4-{[(4-methyl-2-oxo-2H-chromen-7-yl)oxy]methyl}-1H-pyrazol-1-yl)benzonitrile

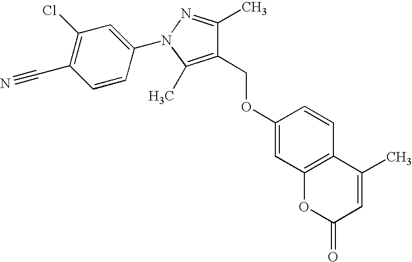

Using 4-methylumbelliferone as a starting material and in the same manner as in Example 142, the title compound was obtained.

MS (ESI+, m/e) 420(M+1).

### Example 193

### 2-chloro-4-{4-[(4-cyclohexylphenoxy)methyl]-3,5-dimethyl-1H-pyrazol-1-yl}benzonitrile

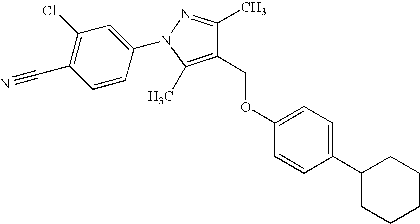

Using 4-cyclohexylphenol as a starting material and in the same manner as in Example 142, the title compound was obtained.

MS (ESI+, m/e) 420(M+1).

### Example 194

### 2-chloro-4-(3,5-dimethyl-4-{[4-(2-oxopyrrolidin-1-yl)phenoxy]methyl}-1H-pyrazol-1-yl)benzonitrile

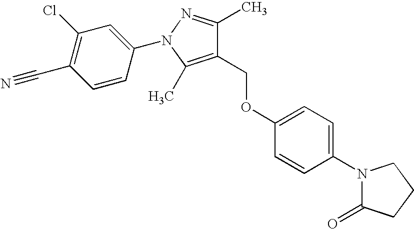

Using 1-(4-hydroxyphenyl)pyrrolidin-2-one as a starting material and in the same manner as in Example 142, the title compound was obtained.

MS (ESI+, m/e) 421(M+1).

¹H-NMR (DMSO-d₆) δ:2.01-2.09 (2H, m), 2.24 (3H, s), 2.44 (3H, s), 2.44-2.48 (2H, m), 3.79 (2H, t), 4.94 (2H, s), 7.03 (2H, d), 7.56 (2H, d), 7.76 (1H, dd), 7.97 (1H, d), 8.11 (1H, d).

### Example 195

### 2-chloro-4-(3,5-dimethyl-4-{[4-(trifluoromethoxy)phenoxy]methyl}-1H-pyrazol-1-yl)benzonitrile

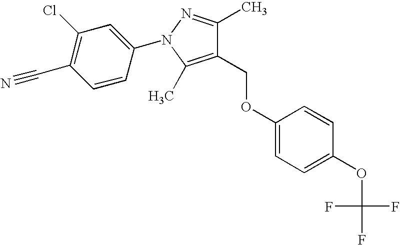

Using 4-(trifluoromethoxy)phenol as a starting material and in the same manner as in Example 142, the title compound was obtained.

MS (ESI+, m/e) 422(M+1).

### Example 196

### 2-chloro-4-(4-{[4-fluoro-2-(1H-pyrazol-3-yl)phenoxy]methyl}-3,5-dimethyl-1H-pyrazol-1-yl)benzonitrile

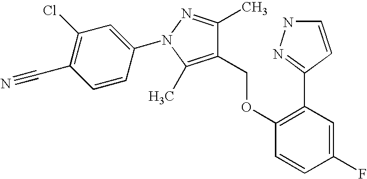

Using 3-(3-fluoro-6-hydroxyphenyl)pyrazole as a starting material and in the same manner as in Example 142, the title compound was obtained.

MS (ESI+, m/e) 422(M+1).

### Example 197

### 2-chloro-4-(3,5-dimethyl-4-{[4-(1,2,3-thiadiazol-4-yl)phenoxy]methyl}-1H-pyrazol-1-yl)benzonitrile

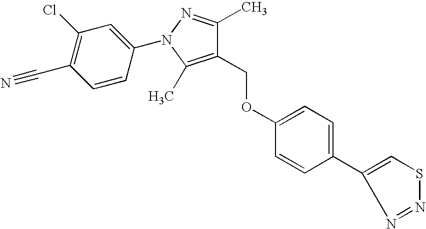

Using 4-(1,2,3-thiadiazol-4-yl)phenol as a starting material and in the same manner as in Example 142, the title compound was obtained.

MS (ESI+, m/e) 422(M+1).

### Example 198

### 2-chloro-4-{4-[(4-fluoro-2-isoxazol-5-ylphenoxy)methyl]-3,5-dimethyl-1H-pyrazol-1-yl}benzonitrile

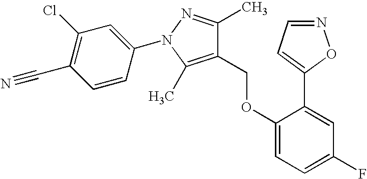

Using 5-(3-fluoro-6-hydroxyphenyl)isoxazole as a starting material and in the same manner as in Example 142, the title compound was obtained.

MS (ESI+, m/e) 423(M+1).

### Example 199

### 2-chloro-4-{3,5-dimethyl-4-[(3-morpholin-4-ylphenoxy)methyl]-1H-pyrazol-1-yl}benzonitrile

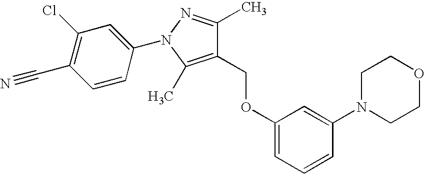

Using 3-morpholinophenol as a starting material and in the same manner as in Example 142, the title compound was obtained.

MS (ESI+, m/e) 423(M+1).

### Example 200

### 2-chloro-4-(4-{[4-fluoro-3-(trifluoromethoxy)phenoxy]methyl}-3,5-dimethyl-1H-pyrazol-1-yl)benzonitrile

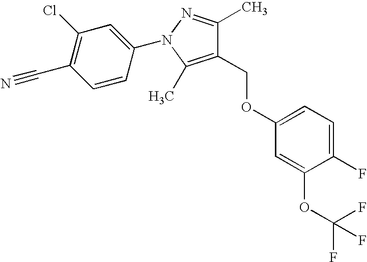

Using 4-fluoro-3-(trifluoromethoxy)phenol as a starting material and in the same manner as in Example 142, the title compound was obtained.

MS (ESI+, m/e) 440(M+1).

¹H-NMR (DMSO-d₆) δ:2.24 (3H, s), 2.44 (3H, s), 4.99 (2H, s), 7.12 (1H, dt), 7.26 (1H, dd), 7.47 (1H, t), 7.76 (1H, dd), 7.97 (1H, d), 8.12 (1H, d).

### Example 201

### 2-chloro-4-(4-{[4-fluoro-3-(trifluoromethyl)phenoxy]methyl}-3,5-dimethyl-1H-pyrazol-1-yl)benzonitrile

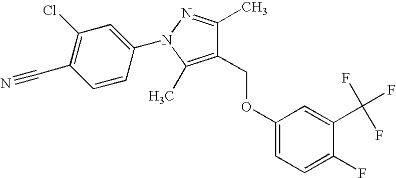

Using 4-fluoro-3-(trifluoromethyl)phenol as a starting material and in the same manner as in Example 142, the title compound was obtained.

MS (ESI+, m/e) 424(M+1).

### Example 202

### 2-chloro-4-{3,5-dimethyl-4-[(4-phenoxyphenoxy)methyl]-1H-pyrazol-1-yl}benzonitrile

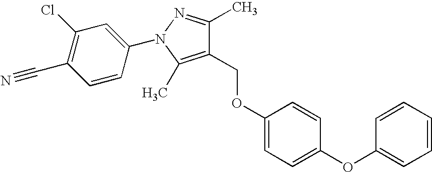

Using 4-phenoxyphenol as a starting material and in the same manner as in Example 142, the title compound was obtained.

MS (ESI+, m/e) 430(M+1).

### Example 203

### 2-chloro-4-[3,5-dimethyl-4-({4-[(trifluoromethyl)sulfanyl]phenoxy}methyl)-1H-pyrazol-1-yl]benzonitrile

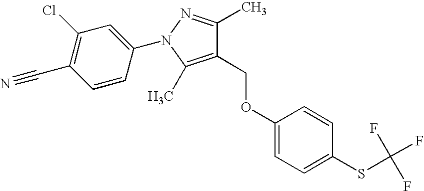

Using 4-(trifluoromethylthio)phenol as a starting material and in the same manner as in Example 142, the title compound was obtained.

MS (ESI+, m/e) 438(M+1).

### Example 204

### 4-(4-{[3,5-bis(trifluoromethyl)phenoxy]methyl}-3,5-dimethyl-1H-pyrazol-1-yl)-2-chlorobenzonitrile

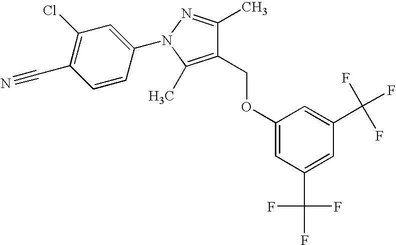

Using 3,5-bis(trifluoromethyl)phenol as a starting material and in the same manner as in Example 142, the title compound was obtained.

MS (ESI+, m/e) 474(M+1).

### Example 205

### 2-chloro-4-{4-[(4-fluorophenoxy)methyl]-3,5-dimethyl-1H-pyrazol-1-yl}benzonitrile

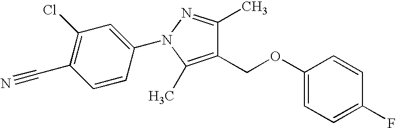

A resin reagent•PS-triphenylphosphine (173 mg) was added to a polypropylene tube equipped with a filter and a cap for preconditioning. Then, a solution (0.303M, 0.685 ml) of 4-fluorophenol in THF and a solution (0.200M, 0.685 ml) of 2-chloro-4-[4-(hydroxymethyl)-3,5-dimethyl-1H-pyrazol-1-yl]benzonitrile synthesized in Reference Example 21 in THF were added, and the mixture was stirred. A solution (0.511M, 0.685 ml) of di-tert-butyl azodicarboxylate in THF was added, and the mixture was stirred at room temperature for 23 hr. The reaction mixture was filtered and washed to remove the resin reagent and the filtrate was concentrated. The residue was purified by reversed-phase preparative HPLC (Gilson, Inc. UniPoint system, YMC ODS column 30×75 millimeter, 0.1% TFA containing acetonitrile-water [5:95-100:0]), and the obtained fraction was concentrated and recrystallized from water-acetonitrile to give the title compound (16.0 mg).

MS (ESI+, m/e) 356(M+1)

¹H-NMR(DMSO-d₆) δ:2.23 (3H, s), 2.43 (3H, s), 4.92 (2H, s), 7.01-7.08 (2H, m), 7.10-7.19 (2H, m), 7.76 (1H, dd), 7.96 (1H, d), 8.11 (1H, d).

### Example 206

### 4-{4-[(4-fluorophenoxy)methyl]-3,5-dimethyl-1H-pyrazol-1-yl}benzonitrile

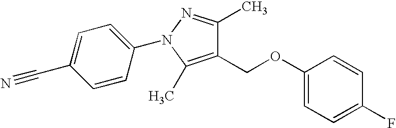

A resin reagent•PS-triphenylphosphine (358 mg) was added to a polypropylene tube equipped with a filter and a cap for preconditioning. Then, a solution (0.300M, 1.50 ml) of 4-fluorophenol in THF and a solution (0.200M, 1.50 ml) of 4-[4-(hydroxymethyl)-3,5-dimethyl-1H-pyrazol-1-yl]benzonitrile synthesized in Reference Example 26 in THF were added, and the mixture was stirred. A solution (0.500M, 1.50 ml) of di-tert-butyl azodicarboxylate in THF was added, and the mixture was stirred at room temperature for 3.5 hr. The reaction mixture was filtered and washed to remove the resin reagent and the filtrate was concentrated. The residue was purified by reversed-phase preparative HPLC (Gilson, Inc. UniPoint system, YMC ODS column 30×75 millimeter, 0.1% TFA containing acetonitrile-water [5:95-100:0]), and the obtained fraction was concentrated and crystallized from methanol to give the title compound (22.8 mg).

MS (ESI+, m/e) 322(M+1)

¹H-NMR(DMSO-d₆) δ:2.23 (3H, s), 2.39 (3H, s), 4.92 (2H, s), 7.05 (2H, dd), 7.14 (2H, dd), 7.76 (2H, d), 7.98 (2H, d).

The syntheses in the following Examples 207-286 were performed using the following instruments and conditions.
dispensing of reagents
Tecan Ltd, Genesis RSP-150
LC-MS analysis
measurement device: Waters Corporation LC-MS system
HPLC unit: Agilent HP1100
MS unit: Micromass ZMD
column: CAPCELL PAK C18 UG120, S-3 μm, 1.5×35 mm (Shiseido Co., Ltd.)
solvent: SOLUTION A; 0.05% trifluoroacetic acid-containing water, SOLUTION B; 0.04% trifluoroacetic acid-containing acetonitrile
gradient cycle: 0.00 min (SOLUTION A/SOLUTION B=90/10), 2.00 min (SOLUTION A/SOLUTION B=5/95), 2.75 min (SOLUTION A/SOLUTION B=5/95), 2.76 min (SOLUTION A/SOLUTION B=90/10), 3.60 min (SOLUTION A/SOLUTION B=90/10)
injection volume: 2 μL, flow rate: 0.5 mL/min, detection method: UV 220 nm
MS conditions ionization method: ESI
purification by preparative HPLC
instrument: Gilson, Inc. high throughput purification system
column: YMC CombiPrep HydrospHere C18 S-5 μm, 19×50 mm
solvent: SOLUTION A; 0.1% trifluoroacetic acid containing
water, SOLUTION B; 0.1% trifluoroacetic acid-containing acetonitrile
gradient cycle: 0.00 min (SOLUTION A/SOLUTION B=95/5), 1.00 min (SOLUTION A/SOLUTION B=95/5), 5.20 min (SOLUTION A/SOLUTION B=5/95), 6.40 min (SOLUTION A/SOLUTION B=5/95), 6.50 min (SOLUTION A/SOLUTION B=95/5), 6.60 min (SOLUTION A/SOLUTION B=95/5)
flow rate: 20 mL/min, detection: UV 220 nm

### Example 207

### 2-{[1-(3-chloro-4-cyanophenyl)-3,5-dimethyl-1H-pyrazol-4-yl]methyl}-N-cyclopropylbenzamide

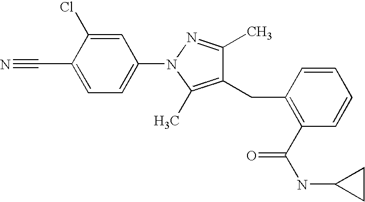

A 0.12 mol/l solution (0.50 ml, 60 μmol) of 2-{[1-(3-chloro-4-cyanophenyl)-3,5-dimethyl-1H-pyrazol-4-yl]methyl}benzoic acid synthesized in Example 90 in DMF, a 0.24 mol/l solution (0.50 ml, 120 μmol) of cyclopropylamine in DMF and a 0.18 mol/l suspension (0.50 ml, 90 μmol) of 4-(4,6-dimethoxy-1,3,5-triazin-2-yl)-4-methylmorpholin-4-ium chloride in DMF were mixed, and the mixture was stirred at room temperature for 24 hr. The reaction mixture was cooled to room temperature, and ethyl acetate (3 ml) and 2% aqueous sodium hydrogen carbonate solution (1.5 ml) were added. The mixture was extracted, and the organic layer was separated by upper layer phase sep tube (manufactured by Wako Pure Chemical Industries, Ltd.). The solvent was evaporated under reduced pressure, and the residue was dissolved in DMSO-MeOH (1:1, 1 ml) and purified by preparative HPLC to give the title compound.

yield :13.5 mg

MS (ESI+, m/e) 405 (M+1)

### Example 208

### N-butyl-2-{[1-(3-chloro-4-cyanophenyl)-3,5-dimethyl-1H-pyrazol-4-yl]methyl}benzamide

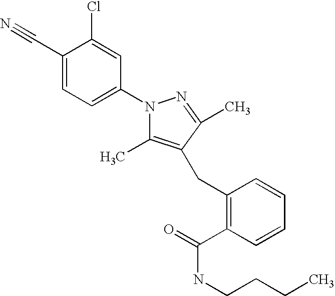

Using butylamine as a starting material and in the same manner as in Example 207, the title compound was obtained.

MS (ESI+, m/e) 421 (M+1)

### Example 209

### N-tert-butyl-2-{[1-(3-chloro-4-cyanophenyl)-3,5-dimethyl-1H-pyrazol-4-yl]methyl}benzamide

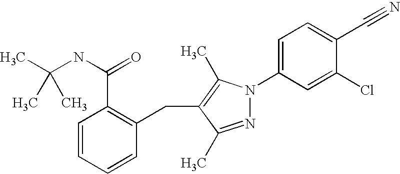

Using tert-butylamine as a starting material and in the same manner as in Example 207, the title compound was obtained.

MS (ESI+, m/e) 421 (M+1)

### Example 210

### 2-{[1-(3-chloro-4-cyanophenyl)-3,5-dimethyl-1H-pyrazol-4-yl]methyl}-N-(3-hydroxypropyl)benzamide

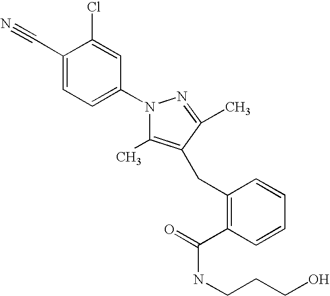

Using 3-aminopropanol as a starting material and in the same manner as in Example 207, the title compound was obtained.

MS (ESI+, m/e) 423 (M+1)

### Example 211

### 2-{[1-(3-chloro-4-cyanophenyl)-3,5-dimethyl-1H-pyrazol-4-yl]methyl}-N-cyclopentylbenzamide

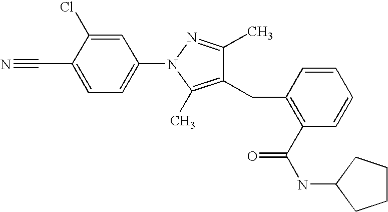

Using cyclopentylamine as a starting material and in the same manner as in Example 207, the title compound was obtained.

MS (ESI+, m/e) 433 (M+1)

### Example 212

### 2-{[1-(3-chloro-4-cyanophenyl)-3,5-dimethyl-1H-pyrazol-4-yl]methyl}-N-(furan-2-ylmethyl)benzamide

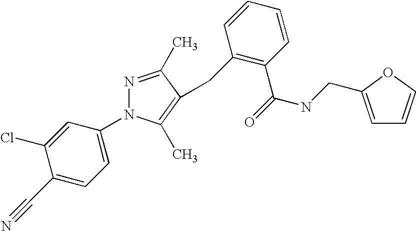

Using furfurylamine as a starting material and in the same manner as in Example 207, the title compound was obtained.

MS (ESI+, m/e) 445 (M+1)

### Example 213

### N-benzyl-2-{[1-(3-chloro-4-cyanophenyl)-3,5-dimethyl-1H-pyrazol-4-yl]methyl}benzamide

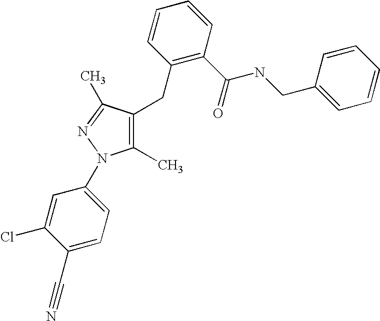

Using benzylamine as a starting material and in the same manner as in Example 207, the title compound was obtained.

MS (ESI+, m/e) 455 (M+1)

### Example 214

### 2-{[1-(3-chloro-4-cyanophenyl)-3,5-dimethyl-1H-pyrazol-4-yl]methyl}-N-(2-hydroxyethyl)benzamide

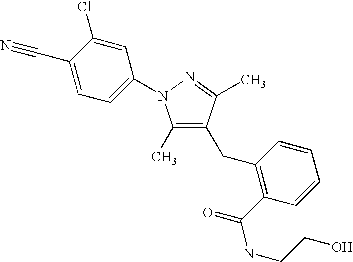

Using 2-aminoethanol as a starting material and in the same manner as in Example 207, the title compound was obtained.

MS (ESI+, m/e) 409 (M+1)

### Example 215

### 2-{[1-(3-chloro-4-cyanophenyl)-3,5-dimethyl-1H-pyrazol-4-yl]methyl}-N-(2-methoxyethyl)benzamide

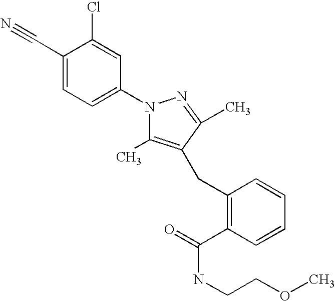

Using 2-methoxyethaneamine as a starting material and in the same manner as in Example 207, the title compound was obtained.

MS (ESI+, m/e) 423 (M+1)

### Example 216

### 2-{[1-(3-chloro-4-cyanophenyl)-3,5-dimethyl-1H-pyrazol-4-yl]methyl}-N-(2-hydroxy-1,1-dimethylethyl)benzamide

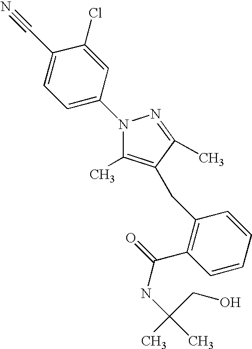

Using 2-amino-2-methyl-1-propanol as a starting material and in the same manner as in Example 207, the title compound was obtained.

MS (ESI+, m/e) 437 (M+1)

### Example 217

### 2-{[1-(3-chloro-4-cyanophenyl)-3,5-dimethyl-1H-pyrazol-4-yl]methyl}-N-[2-(2-hydroxyethoxy)ethyl]benzamide

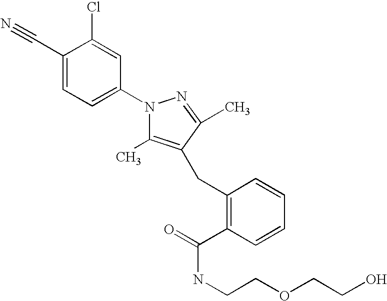

Using 2-(2-aminoethoxy)ethanol as a starting material and in the same manner as in Example 207, the title compound was obtained.

MS (ESI+, m/e) 453 (M+1)

### Example 218

### N-(2-amino-2-oxoethyl)-2-{[1-(3-chloro-4-cyanophenyl)-3,5-dimethyl-1H-pyrazol-4-yl]methyl}benzamide

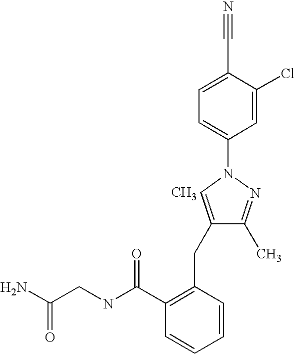

Using glycinamide hydrochloride and triethylamine as starting materials and in the same manner as in Example 207, the title compound was obtained.

MS (ESI+, m/e) 422 (M+1)

### Example 219

### 2-{[1-(3-chloro-4-cyanophenyl)-3,5-dimethyl-1H-pyrazol-4-yl]methyl}-N-(1-methylethyl)benzamide

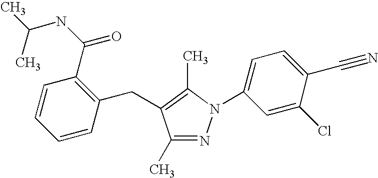

Using isopropylamine as a starting material and in the same manner as in Example 207, the title compound was obtained.

MS (ESI+, m/e) 407 (M+1)

### Example 220

### 2-chloro-4-{3,5-dimethyl-4-[2-(pyrrolidin-1-ylcarbonyl)benzyl]-1H-pyrazol-1-yl}benzonitrile

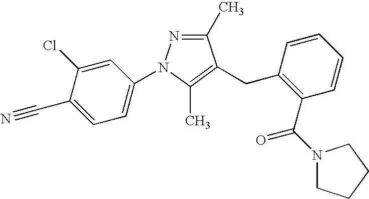

Using pyrrolidine as a starting material and in the same manner as in Example 207, the title compound was obtained.

MS (ESI+, m/e) 419 (M+1)

### Example 221

### 2-{[1-.(3-chloro-4-cyanophenyl)-3,5-dimethyl-1H-pyrazol-4-yl]methyl}-N,N-diethylbenzamide

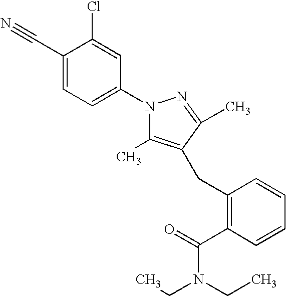

Using diethylamine as a starting material and in the same manner as in Example 207, the title compound was obtained.

MS (ESI+, m/e) 421 (M+1)

### Example 222

### 2-chloro-4-{3,5-dimethyl-4-[2-(morpholin-4-ylcarbonyl)benzyl]-1H-pyrazol-1-yl}benzonitrile

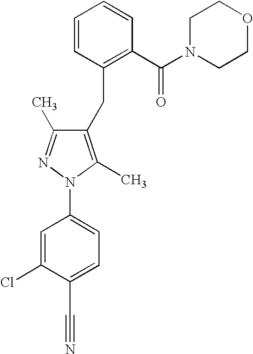

Using morpholine as a starting material and in the same manner as in Example 207, the title compound was obtained.

MS (ESI+, m/e) 435 (M+1)

### Example 223

### 2-{[1-(3-chloro-4-cyanophenyl)-3,5-dimethyl-1H-pyrazol-4-yl]methyl}-N-(2-methoxyethyl)-N-methylbenzamide

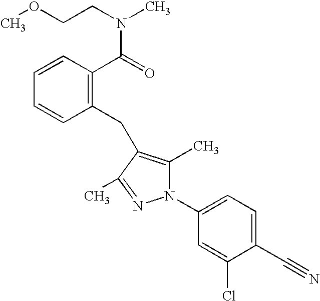

Using N-(2-methoxyethyl)methylamine as a starting material and in the same manner as in Example 207, the title compound was obtained.

MS (ESI+, m/e) 437 (M+1)

### Example 224

### 2-chloro-4-[4-(2-{[(2S)-2-(hydroxymethyl)pyrrolidin-1-yl]carbonyl}benzyl)-3,5-dimethyl-1H-pyrazol-1-yl]benzonitrile

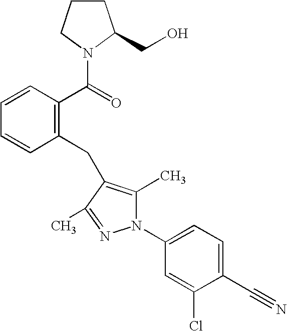

Using (2S)-2-(hydroxymethyl)pyrrolidine as a starting material and in the same manner as in Example 207, the title compound was obtained.

MS (ESI+, m/e) 449 (M+1)

### Example 225

### 2-chloro-4-(4-{2-[(4-hydroxypiperidin-1-yl)carbonyl]benzyl}-3,5-dimethyl-1H-pyrazol-1-yl)benzonitrile

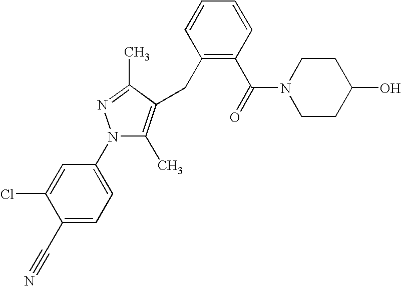

Using 4-hydroxypiperidine as a starting material and in the same manner as in Example 207, the title compound was obtained.

MS (ESI+, m/e) 449 (M+1)

### Example 226

### 1-[(2-{[1-(3-chloro-4-cyanophenyl)-3,5-dimethyl-1H-pyrazol-4-yl]methyl}phenyl)carbonyl]piperidine-4-carboxamide

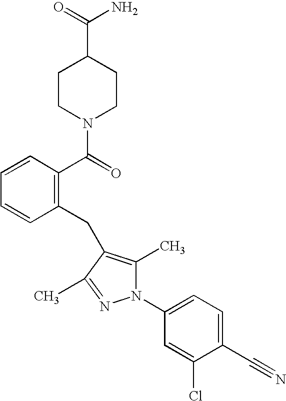

Using 4-piperidinecarboxamide as a starting material and in the same manner as in Example 207, the title compound was obtained.

MS (ESI+, m/e) 476 (M+1)

### Example 227

### 2-chloro-4-[4-(2-{[4-(2-hydroxyethyl)piperidin-1-yl]carbonyl}benzyl)-3,5-dimethyl-1H-pyrazol-1-yl]benzonitrile

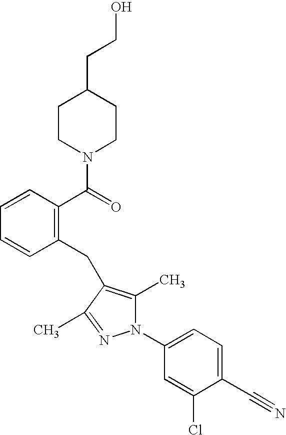

Using 4-(2-hydroxyethyl)piperidine as a starting material and in the same manner as in Example 207, the title compound was obtained.

MS (ESI+, m/e) 477 (M+1)

### Example 228

### (±)-2-chloro-4-(4-{2-[(3-hydroxypyrrolidin-1-yl)carbonyl]benzyl}-3,5-dimethyl-1H-pyrazol-1-yl)benzonitrile

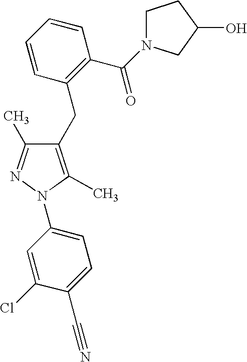

Using (±)-3-hydroxypyrrolidine as a starting material and in the same manner as in Example 207, the title compound was obtained.

MS (ESI+, m/e) 435 (M+1)

### Example 229

### 2-{[1-(3-chloro-4-cyanophenyl)-3,5-dimethyl-1H-pyrazol-4-yl]methyl}-N-[2-(dimethylamino)ethyl]benzamide trifluoroacetate

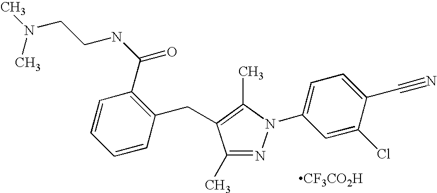

Using 2-(dimethylamino)ethylamine as a starting material and in the same manner as in Example 207, the title compound was obtained.

MS (ESI+, m/e) 436 (M+1)

### Example 230

### 2-{[1-(3-chloro-4-cyanophenyl)-3,5-dimethyl-1H-pyrazol-4-yl]methyl}-N-[3-(dimethylamino)propyl]benzamide trifluoroacetate

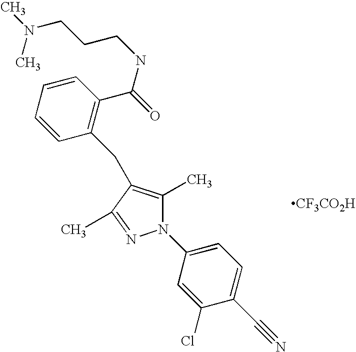

Using 3-(dimethylamino)propylamine as a starting material and in the same manner as in Example 207, the title compound was obtained.

MS (ESI+, m/e) 450 (M+1)

### Example 231

### 2-{[1-(3-chloro-4-cyanophenyl)-3,5-dimethyl-1H-pyrazol-4-yl]methyl}-N-(pyridin-2-ylmethyl)benzamide trifluoroacetate

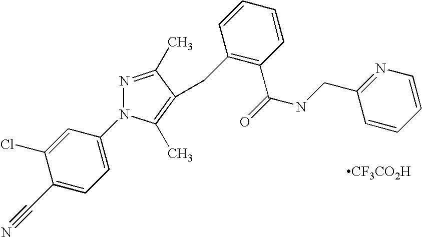

Using 2-(aminomethyl)pyridine as a starting material and in the same manner as in Example 207, the title compound was obtained.

MS (ESI+, m/e) 456 (M+1)

### Example 232

### 2-chloro-4-(3,5-dimethyl-4-{2-[(4-methylpiperazin-1-yl)carbonyl]benzyl}-1H-pyrazol-1-yl)benzonitrile trifluoroacetate

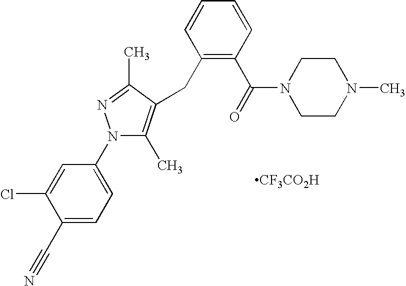

Using 1-methylpiperazine as a starting material and in the same manner as in Example 207, the title compound was obtained.

MS (ESI+, m/e) 448 (M+1)

### Example 233

### (±)-2-chloro-4-[4-(2-{[3-(dimethylamino)pyrrolidin-1-yl]carbonyl}benzyl)-3,5-dimethyl-1H-pyrazol-1-yl]benzonitrile trifluoroacetate

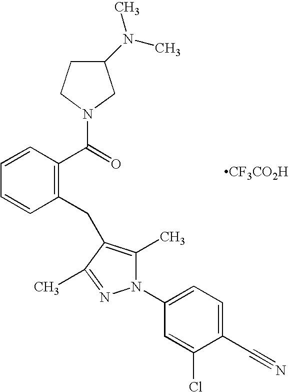

Using (±)-3-(dimethylamino)pyrrolidine as a starting material and in the same manner as in Example 207, the title compound was obtained.

MS (ESI+, m/e) 462 (M+1)

### Example 234

### 2-chloro-4-[4-(2-{[4-(2-hydroxyethyl)piperazin-1-yl]carbonyl}benzyl)-3,5-dimethyl-1H-pyrazol-1-yl]benzonitrile trifluoroacetate

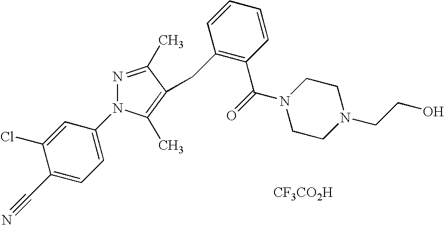

Using 1-(2-hydroxyethyl)piperazine as a starting material and in the same manner as in Example 207, the title compound was obtained.

MS (ESI+, m/e) 478 (M+1)

### Example 235

### 2-{[1-(3-chloro-4-cyanophenyl)-3,5-dimethyl-1H-pyrazol-4-yl]methyl}-N-phenylbenzamide

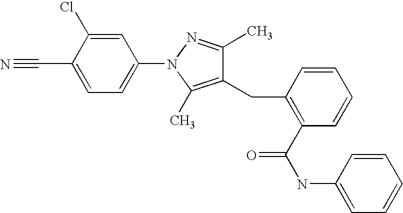

Using aniline as a starting material and in the same manner as in Example 207, the title compound was obtained.

MS (ESI+, m/e) 441 (M+1)

### Example 236

### 2-{[1-(3-chloro-4-cyanophenyl)-3,5-dimethyl-1H-pyrazol-4-yl]methyl}-N-1,3-thiazol-2-ylbenzamide

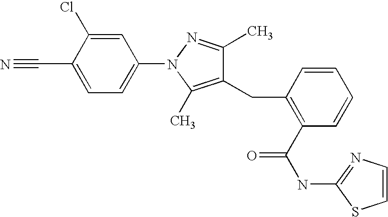

Using 2-aminothiazole as a starting material and in the same manner as in Example 207, the title compound was obtained.

MS (ESI+, m/e) 448 (M+1)

### Example 237

### 3-{[1-(3-chloro-4-cyanophenyl)-3,5-dimethyl-1H-pyrazol-4-yl]methyl}-N-cyclopropylbenzamide

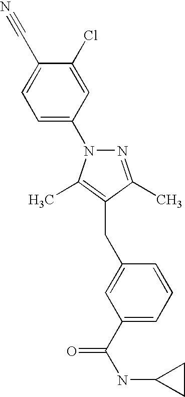

A 0.12 mol/l solution (0.50 ml, 60 μmol) of 3-{[1-(3-chloro-4-cyanophenyl)-3,5-dimethyl-1H-pyrazol-4-yl]methyl}benzoic acid synthesized in Example 91 in DMF, a 0.24 mol/l solution (0.50 ml, 120 μmol) of cyclopropylamine in DMF and a 0.18 mol/l suspension (0.50 ml, 90 μmol) of 4-(4,6-dimethoxy-1,3,5-triazin-2-yl)-4-methylmorpholin-4-ium chloride in DMF were mixed, and the mixture was stirred at room temperature for 24 hr. The reaction mixture was cooled to room temperature, and ethyl acetate (3 ml) and 2% aqueous sodium hydrogen carbonate solution (1.5 ml) were added. The mixture was extracted, and the organic layer was separated by upper layer phase sep tube (manufactured by Wako Pure Chemical Industries, Ltd.). The solvent was evaporated under reduced pressure, and the residue was dissolved in DMSO-MeOH (1:1, 1 ml) and purified by preparative HPLC to give the title compound.

yield :13.3 mg

MS (ESI+, m/e):405(M+1)

### Example 238

### N-butyl-3-{[1-(3-chloro-4-cyanophenyl)-3,5-dimethyl-1H-pyrazol-4-yl]methyl}benzamide

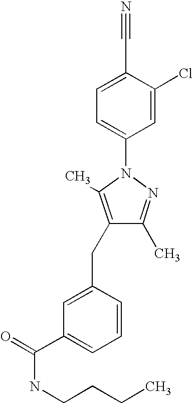

Using butylamine as a starting material and in the same manner as in Example 237, the title compound was obtained.

MS (ESI+, m/e) 421 (M+1)

### Example 239

### N-tert-butyl-3-{[1-(3-chloro-4-cyanophenyl)-3,5-dimethyl-1H-pyrazol-4-yl]methyl}benzamide

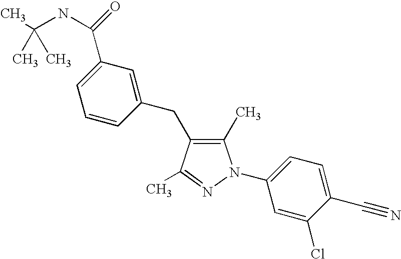

Using tert-butylamine as a starting material and in the same manner as in Example 237, the title compound was obtained.

MS (ESI+, m/e) 421 (M+1)

### Example 240

### 3-{[1-(3-chloro-4-cyanophenyl)-3,5-dimethyl-1H-pyrazol-4-yl]methyl}-N-(3-hydroxypropyl)benzamide

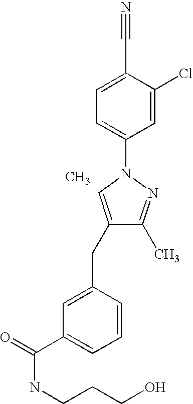

Using 3-aminopropanol as a starting material and in the same manner as in Example 237, the title compound was obtained.

MS (ESI+, m/e) 423 (M+1)

### Example 241

### 3-{[1-(3-chloro-4-cyanophenyl)-3,5-dimethyl-1H-pyrazol-4-yl]methyl}-N-cyclopentylbenzamide

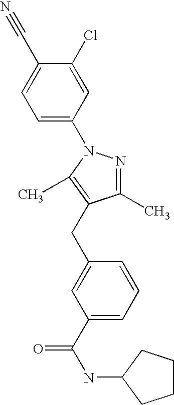

Using cyclopentylamine as a starting material and in the same manner as in Example 237, the title compound was obtained.

MS (ESI+, m/e) 433 (M+1)

### Example 242

### 3-{[1-(3-chloro-4-cyanophenyl)-3,5-dimethyl-1H-pyrazol-4-yl]methyl}-N-(furan-2-ylmethyl)benzamide

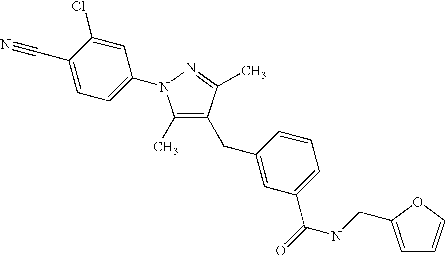

Using furfurylamine as a starting material and in the same manner as in Example 237, the title compound was obtained.

MS (ESI+, m/e) 445 (M+1)

### Example 243

### N-benzyl-3-{[1-(3-chloro-4-cyanophenyl)-3,5-dimethyl-1H-pyrazol-4-yl]methyl}benzamide

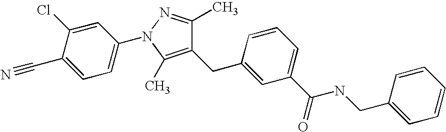

Using benzylamine as a starting material and in the same manner as in Example 237, the title compound was obtained.

MS (ESI+, m/e) 455 (M+1)

### Example 244

### 3-{[1-(3-chloro-4-cyanophenyl)-3,5-dimethyl-1H-pyrazol-4-yl]methyl}-N-(2-hydroxyethyl)benzamide

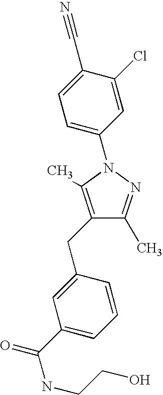

Using 2-aminoethanol as a starting material and in the same manner as in Example 237, the title compound was obtained.

MS (ESI+, m/e) 409 (M+1)

### Example 245

### 3-{[1-(3-chloro-4-cyanophenyl)-3,5-dimethyl-1H-pyrazol-4-yl]methyl}-N-(2-methoxyethyl)benzamide

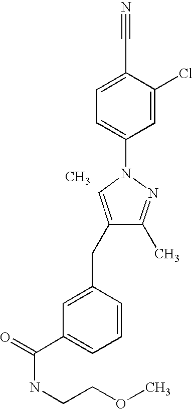

Using 2-methoxyethaneamine as a starting material and in the same manner as in Example 237, the title compound was obtained.

MS (ESI+, m/e) 423 (M+1)

### Example 246

### 3-{[1-(3-chloro-4-cyanophenyl)-3,5-dimethyl-1H-pyrazol-4-yl]methyl}-N-(2-hydroxy-1,1-dimethylethyl)benzamide

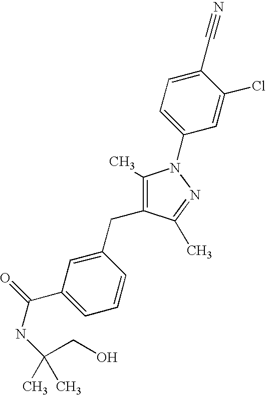

Using 2-amino-2-methyl-1-propanol as a starting material and in the same manner as in Example 237, the title compound was obtained.

MS (ESI+, m/e) 437 (M+1)

### Example 247

### 3-{[1-(3-chloro-4-cyanophenyl)-3,5-dimethyl-1H-pyrazol-4-yl]methyl}-N-[2-(2-hydroxyethoxy)ethyl]benzamide

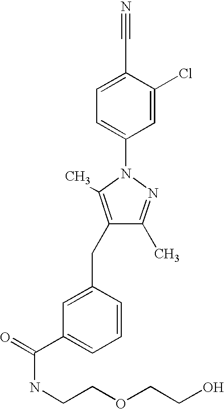

Using 2-(2-aminoethoxy)ethanol as a starting material and in the same manner as in Example 237, the title compound was obtained.

MS (ESI+, m/e) 453 (M+1)

### Example 248

### N-(2-amino-2-oxoethyl)-3-{[l-(3-chloro-4-cyanophenyl)-3,5-dimethyl-1H-pyrazol-4-yl]methyl}benzamide

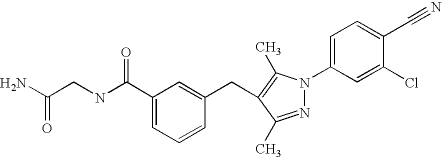

Using glycinamide hydrochloride and triethylamine as starting materials and in the same manner as in Example 237, the title compound was obtained.

MS (ESI+, m/e) 422 (M+1)

### Example 249

### 3-{[1-(3-chloro-4-cyanophenyl)-3,5-dimethyl-1H-pyrazol-4-yl]methyl}-N-(1-methylethyl)benzamide

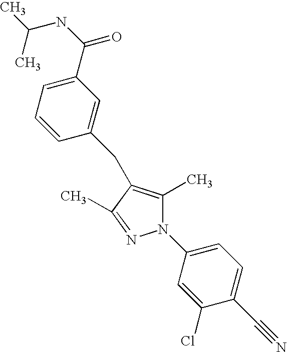

Using isopropylamine as a starting material and in the same manner as in Example 237, the title compound was obtained.

MS (ESI+, m/e) 407 (M+1)

### Example 250

### 2-chloro-4-{3,5-dimethyl-4-[3-(pyrrolidin-1-ylcarbonyl)benzyl]-1H-pyrazol-1-yl}benzonitrile

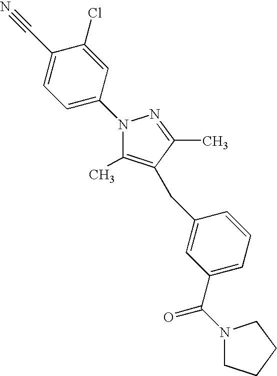

Using pyrrolidine as a starting material and in the same manner as in Example 237, the title compound was obtained.

MS (ESI+, m/e) 419 (M+1)

### Example 251

### 3-{[1-(3-chloro-4-cyanophenyl)-3,5-dimethyl-1H-pyrazol-4-yl]methyl}-N,N-diethylbenzamide

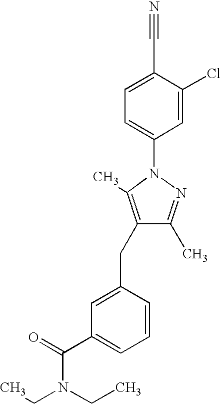

Using diethylamine as a starting material and in the same manner as in Example 237, the title compound was obtained.

MS (ESI+, m/e) 421 (M+1)

### Example 252

### 2-chloro-4-{3,5-dimethyl-4-[3-(morpholin-4-ylcarbonyl)benzyl]-1H-pyrazol-1-yl}benzonitrile

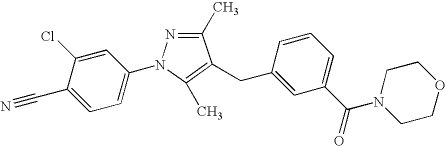

Using morpholine as a starting material and in the same manner as in Example 237, the title compound was obtained.

MS (ESI+, m/e) 435 (M+1)

### Example 253

### 3-{[1-(3-chloro-4-cyanophenyl)-3,5-dimethyl-1H-pyrazol-4-yl]methyl}-N-(2-methoxyethyl)-N-methylbenzamide

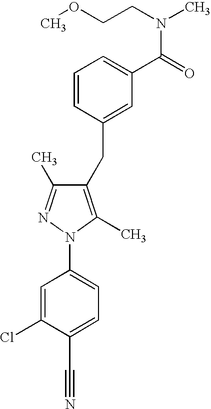

Using N-(2-methoxyethyl)methylamine as a starting material and in the same manner as in Example 237, the title compound was obtained.

MS (ESI+, m/e) 437 (M+1)

### Example 254

### 2-chloro-4-[4-(3-{[(2S)-2-(hydroxymethyl)pyrrolidin-1-yl]carbonyl}benzyl)-3,5-dimethyl-1H-pyrazol-1-yl]benzonitrile

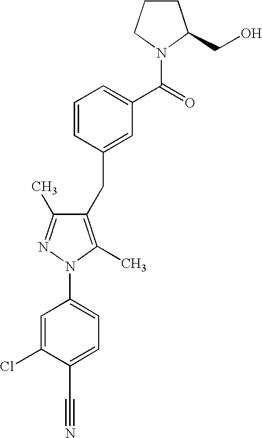

Using (2S)-2-(hydroxymethyl)pyrrolidine as a starting material and in the same manner as in Example 237, the title compound was obtained.

MS (ESI+, m/e) 449 (M+1)

### Example 255

### 2-chloro-4-(4-{3-[(4-hydroxypiperidin-1-yl)carbonyl]benzyl}-3,5-dimethyl-1H-pyrazol-1-yl)benzonitrile

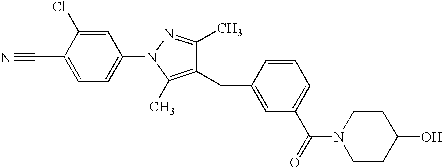

Using 4-hydroxypiperidine as a starting material and in the same manner as in Example 237, the title compound was obtained.

MS (ESI+, m/e) 449 (M+1)

### Example 256

### 1-[(3-{[1-(3-chloro-4-cyanophenyl)-3,5-dimethyl-1H-pyrazol-4-yl]methyl}phenyl)carbonyl]piperidine-4-carboxamide

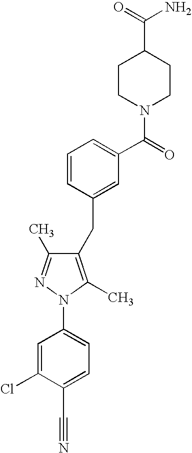

Using 4-piperidinecarboxamide as a starting material and in the same manner as in Example 237, the title compound was obtained.

MS (ESI+, m/e) 476 (M+1)

### Example 257

### 2-chloro-4-[4-(3-{[4-(2-hydroxyethyl)piperidin-1-yl]carbonyl}benzyl)-3,5-dimethyl-1H-pyrazol-1-yl]benzonitrile

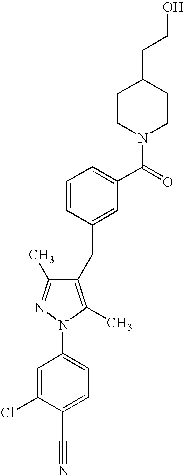

Using 4-(2-hydroxyethyl)piperidine as a starting material and in the same manner as in Example 237, the title compound was obtained.

MS (ESI+, m/e) 477 (M+1)

### Example 258

### (±)-2-chloro-4-(4-{3-[(3-hydroxypyrrolidin-1-yl)carbonyl]benzyl}-3,5-dimethyl-1H-pyrazol-1-yl)benzonitrile

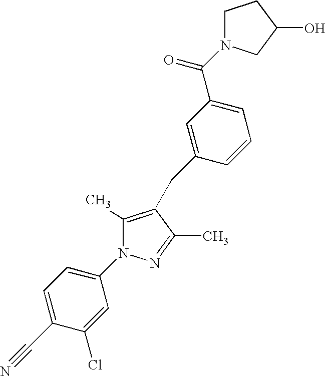

Using (±)-3-hydroxypyrrolidine as a starting material and in the same manner as in Example 237, the title compound was obtained.

MS (ESI+, m/e) 435 (M+1)

### Example 259

### 3-{[1-(3-chloro-4-cyanophenyl)-3,5-dimethyl-1H-pyrazol-4-yl]methyl}-N-[2-(dimethylamino)ethyl]benzamide trifluoroacetate

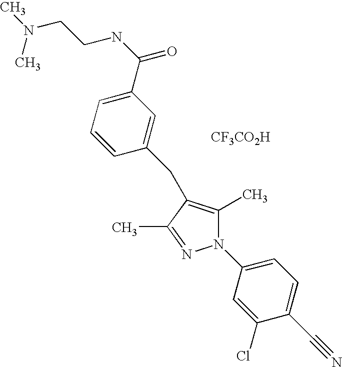

Using 2-(dimethylamino)ethylamine as a starting material and in the same manner as in Example 237, the title compound was obtained.

MS (ESI+, m/e) 436 (M+1)

### Example 260

### 3-{[1-(3-chloro-4-cyanophenyl)-3,5-dimethyl-1H-pyrazol-4-yl]methyl}-N-[3-(dimethylamino)propyl]benzamide trifluoroacetate

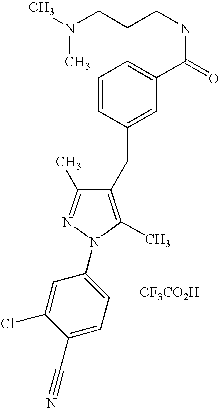

Using 3-(dimethylamino)propylamine as a starting material and in the same manner as in Example 237, the title compound was obtained.

MS (ESI+, m/e) 450 (M+1)

### Example 261

### 3-{[1-(3-chloro-4-cyanophenyl)-3,5-dimethyl-1H-pyrazol-4-yl]methyl}-N-(pyridin-2-ylmethyl)benzamide trifluoroacetate

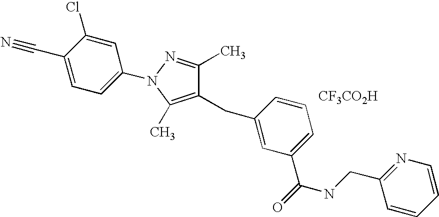

Using 2-(aminomethyl)pyridine as a starting material and in the same manner as in Example 237, the title compound was obtained.

MS (ESI+, m/e) 456 (M+1)

### Example 262

### 2-chloro-4-(3,5-dimethyl-4-{3-[(4-methylpiperazin-1-yl)carbonyl]benzyl}-1H-pyrazol-1-yl)benzonitrile trifluoroacetate

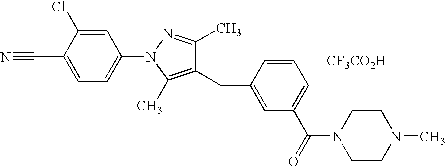

Using 1-methylpiperazine as a starting material and in the same manner as in Example 237, the title compound was obtained.

MS (ESI+, m/e) 448 (M+1)

### Example 263

### (±)-2-chloro-4-[4-(3-{[3-(dimethylamino)pyrrolidin-1-yl]carbonyl}benzyl)-3,5-dimethyl-1H-pyrazol-1-yl]benzonitrile trifluoroacetate

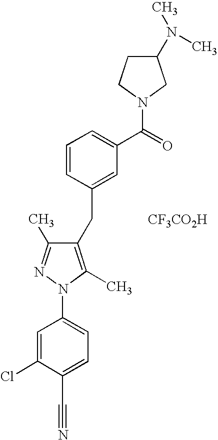

Using (±)-3-(dimethylamino)pyrrolidine as a starting material and in the same manner as in Example 237, the title compound was obtained.

MS (ESI+, m/e) 462 (M+1)

### Example 264

### 2-chloro-4-[4-(3-{[4-(2-hydroxyethyl)piperazin-1-yl]carbonyl}benzyl)-3,5-dimethyl-1H-pyrazol-1-yl]benzonitrile trifluoroacetate

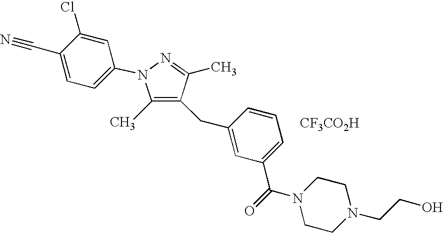

Using 1-(2-hydroxyethyl)piperazine as a starting material and in the same manner as in Example 237, the title compound was obtained.

MS (ESI+, m/e) 478 (M+1)

### Example 265

### 3-{[1-(3-chloro-4-cyanophenyl)-3,5-dimethyl-1H-pyrazol-4-yl]methyl}-N-phenylbenzamide

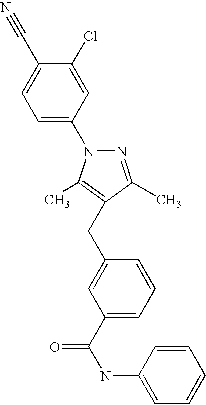

Using aniline as a starting material and in the same manner as in Example 237, the title compound was obtained.

MS (ESI+, m/e) 441 (M+1)

### Example 266

### 4-{[1-(3-chloro-4-cyanophenyl)-3,5-dimethyl-1H-pyrazol-4-yl]methyl}-N-cyclopropylbenzamide

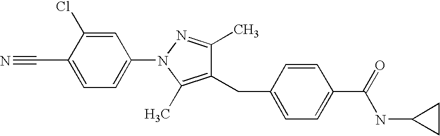

A 0.12 mol/l solution (0.50 ml, 60 μmol) of 4-{[1-(3-chloro-4-cyanophenyl)-3,5-dimethyl-1H-pyrazol-4-yl]methyl}benzoic acid synthesized in Example 92 in DMF, a 0.24 mol/l solution (0.50 ml, 120 μmol) of cyclopropylamine in DMF and a 0.18 mol/l suspension (0.50 ml, 90 μmol) of 4-(4,6-dimethoxy-1,3,5-triazin-2-yl)-4-methylmorpholin-4-ium chloride in DMF were mixed, and the mixture was stirred at room temperature for 24 hr. The reaction mixture was cooled to room temperature, and ethyl acetate (3 ml) and 2% aqueous sodium hydrogen carbonate solution (1.5 ml) were added. The mixture was extracted, and the organic layer was separated by upper layer phase sep tube (manufactured by Wako Pure Chemical Industries, Ltd.). The solvent was evaporated under reduced pressure, and the residue was dissolved in DMSO-MeOH (1:1, 1 ml) and purified by preparative HPLC to give the title compound.

yield: 12.0 mg

MS (ESI+, m/e):405(M+1)

### Example 267

### N-butyl-4-{[1-(3-chloro-4-cyanophenyl)-3,5-dimethyl-1H-pyrazol-4-yl]methyl}benzamide

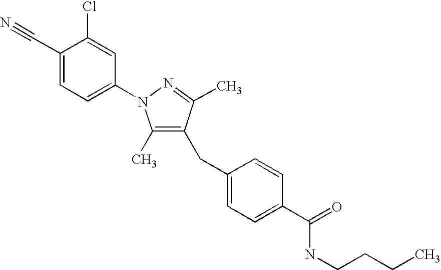

Using butylamine as a starting material and in the same manner as in Example 266, the title compound was obtained.

MS (ESI+, m/e) 423 (M+1)

### Example 268

### 4-{[1-(3-chloro-4-cyanophenyl)-3,5-dimethyl-1H-pyrazol-4-yl]methyl}-N-cyclopentylbenzamide

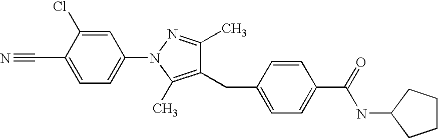

Using cyclopentylamine as a starting material and in the same manner as in Example 266, the title compound was obtained.

MS (ESI+, m/e) 433 (M+1)

### Example 269

### 4-{[1-(3-chloro-4-cyanophenyl)-3,5-dimethyl-1H-pyrazol-4-yl]methyl}-N-(furan-2-ylmethyl)benzamide

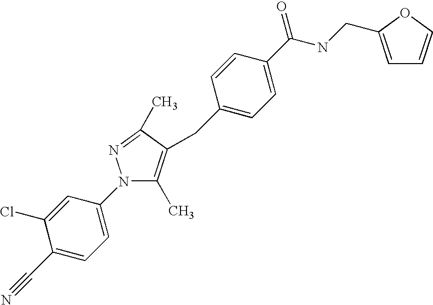

Using furfurylamine as a starting material and in the same manner as in Example 266, the title compound was obtained.

MS (ESI+, m/e) 445 (M+1)

### Example 270

### N-benzyl-4-{[1-(3-chloro-4-cyanophenyl)-3,5-dimethyl-1H-pyrazol-4-yl]methyl}benzamide

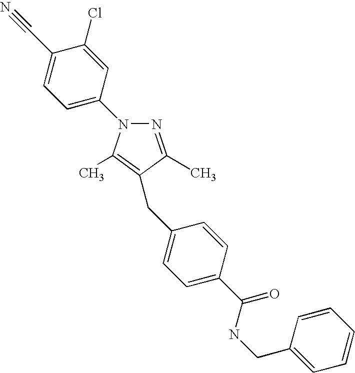

Using benzylamine as a starting material and in the same manner as in Example 266, the title compound was obtained.

MS (ESI+, m/e) 455 (M+1)

### Example 271

### 4-{[1-(3-chloro-4-cyanophenyl)-3,5-dimethyl-1H-pyrazol-4-yl]methyl}-N-(2-hydroxy-1,1-dimethylethyl)benzamide

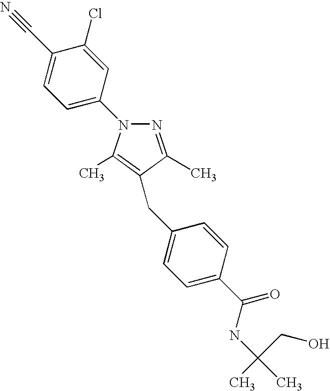

Using 2-amino-2-methyl-1-propanol as a starting material and in the same manner as in Example. 266, the title compound was obtained.

MS (ESI+, m/e) 437 (M+1)

### Example 272

### N-(2-amino-2-oxoethyl)-4-{[1-(3-chloro-4-cyanophenyl)-3,5-dimethyl-1H-pyrazol-4-yl]methyl}benzamide

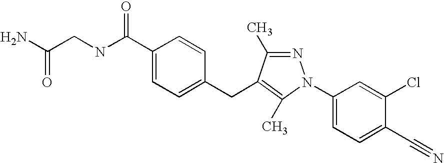

Using glycinamide hydrochloride and triethylamine as starting materials and in the same manner as in Example 266, the title compound was obtained.

MS (ESI+, m/e) 422 (M+1)

### Example 273

### 2-chloro-4-{3,5-dimethyl-4-[4-(pyrrolidin-1-ylcarbonyl)benzyl]-1H-pyrazol-1-yl}benzonitrile

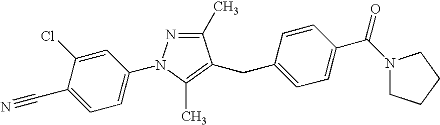

Using pyrrolidine as a starting material and in the same manner as in Example 266, the title compound was obtained.

MS (ESI+, m/e) 419 (M+1)

### Example 274

### 4-{[1-(3-chloro-4-cyanophenyl)-3,5-dimethyl-1H-pyrazol-4-yl]methyl}-N,N-diethylbenzamide

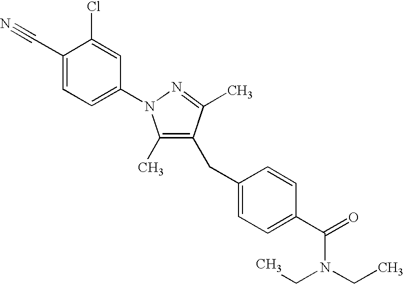

Using diethylamine as a starting material and in the same manner as in Example 266, the title compound was obtained.

MS (ESI+, m/e) 421 (M+1)

### Example 275

### 4-{[1-(3-chloro-4-cyanophenyl)-3,5-dimethyl-1H-pyrazol-4-yl]methyl}-N-(2-methoxyethyl)-N-methylbenzamide

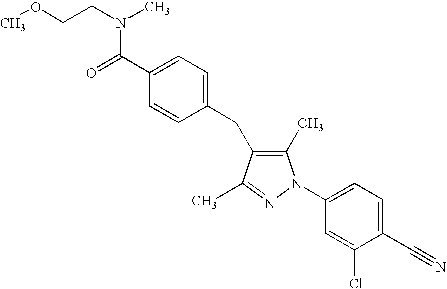

Using N-(2-methoxyethyl)methylamine as a starting material and in the same manner as in Example 266, the title compound was obtained.

MS (ESI+, m/e) 437 (M+1)

### Example 276

### 2-chloro-4-[4-(4-{[(2S)-2-(hydroxymethyl)pyrrolidin-1-yl]carbonyl}benzyl)-3,5-dimethyl-1H-pyrazol-1-yl]benzonitrile

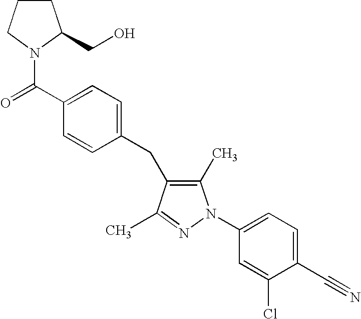

Using (2S)-2-(hydroxymethyl)pyrrolidine as a starting material and in the same manner as in Example 266, the title compound was obtained.

MS (ESI+, m/e) 449 (M+1)

### Example 277

### 1-[ (4-{[1-(3-chloro-4-cyanophenyl)-3,5-dimethyl-1H-pyrazol-4-yl]methyl}phenyl)carbonyl]piperidine-4-carboxamide

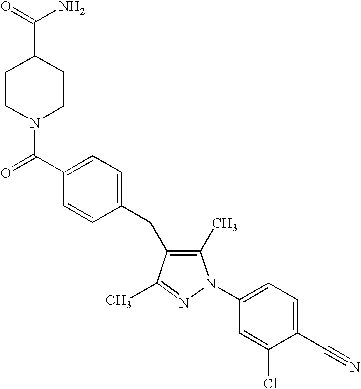

Using 4-piperidinecarboxamide as a starting material and in the same manner as in Example 266, the title compound was obtained.

MS (ESI+, m/e) 476 (M+1)

### Example 278

### 2-chloro-4-[4-(4-{[4-(2-hydroxyethyl)piperidin-1-yl]carbonyl}benzyl)-3,5-dimethyl-1H-pyrazol-1-yl]benzonitrile

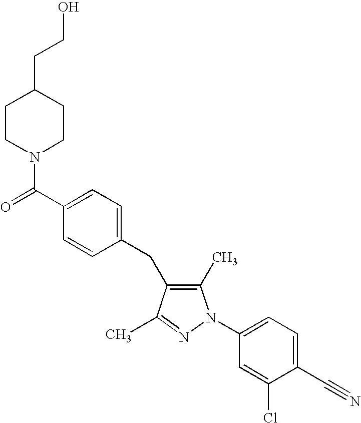

Using 4-(2-hydroxyethyl)piperidine as a starting material and in the same manner as in Example 266, the title compound was obtained.

MS (ESI+, m/e) 477 (M+1)

### Example 279

### (±)-2-chloro-4-(4-{4-[(3-hydroxypyrrolidin-1-yl)carbonyl]benzyl}-3,5-dimethyl-1H-pyrazol-1-yl)benzonitrile

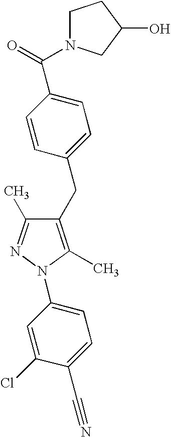

Using (±)-3-hydroxypyrrolidine as a starting material and in the same manner as in Example 266, the title compound was obtained.

MS (ESI+, m/e) 435 (M+1)

### Example 280

### 4-{[1-(3-chloro-4-cyanophenyl)-3,5-dimethyl-1H-pyrazol-4-yl]methyl}-N-[2-(dimethylamino)ethyl]benzamide trifluoroacetate

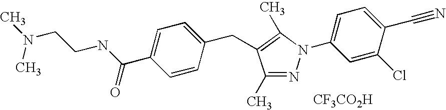

Using 2-(dimethylamino)ethylamine as a starting material and in the same manner as in Example 266, the title compound was obtained.

MS (ESI+, m/e) 436 (M+1)

### Example 281

### 4-{[1-(3-chloro-4-cyanophenyl)-3,5-dimethyl-1H-pyrazol-4-yl]methyl}-N-[3-(dimethylamino)propyl]benzamide trifluoroacetate

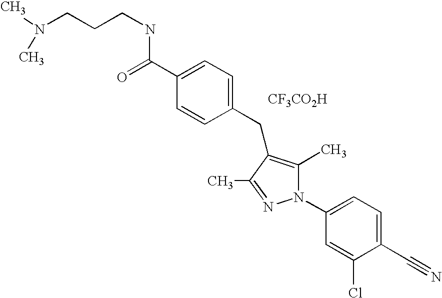

Using 3-(dimethylamino)propylamine as a starting material and in the same manner as in Example 266, the title compound was obtained.

MS (ESI+, m/e) 450 (M+1)

### Example 282

### 2-chloro-4-(3,5-dimethyl-4-{4-[(4-methylpiperazin-1-yl)carbonyl]benzyl}-1H-pyrazol-1-yl)benzonitrile trifluoroacetate

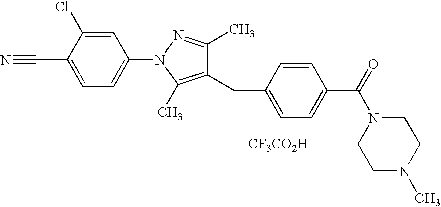

Using 1-methylpiperazine as a starting material and in the same manner as in Example 266, the title compound was obtained.

MS (ESI+, m/e) 448 (M+1)

### Example 283

### (±)-2-chloro-4-[4-(4-{[3-(dimethylamino)pyrrolidin-1-yl]carbonyl}benzyl)-3,5-dimethyl-1H-pyrazol-1-yl]benzonitrile trifluoroacetate

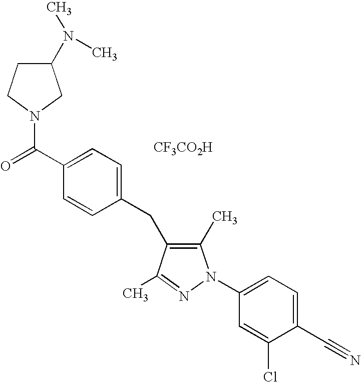

Using (±)-3-(dimethylamino)pyrrolidine as a starting material and in the same manner as in Example 266, the title compound was obtained.

MS (ESI+, m/e) 462 (M+1)

### Example 284

### 2-chloro-4-[4-(4-{[4-(2-hydroxyethyl)piperazin-1-yl]carbonyl}benzyl)-3,5-dimethyl-1H-pyrazol-1-yl]benzonitrile trifluoroacetate

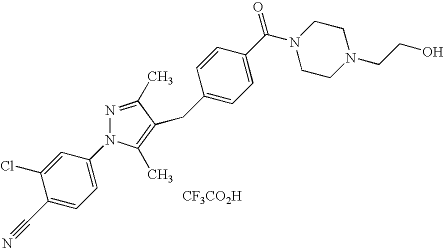

Using 1-(2-hydroxyethyl)piperazine as a starting material and in the same manner as in Example 266, the title compound was obtained.

MS (ESI+, m/e) 478 (M+1)

### Example 285

### 4-{[1-(3-chloro-4-cyanophenyl)-3,5-dimethyl-1H-pyrazol-4-yl]methyl}-N-phenylbenzamide

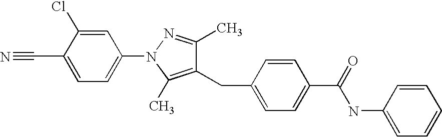

Using aniline as a starting material and in the same manner as in Example 266, the title compound was obtained.

MS (ESI+, m/e) 441 (M+1)

### Example 286

### 4-{[1-(3-chloro-4-cyanophenyl)-3,5-dimethyl-1H-pyrazol-4-yl]methyl}-N-1,3-thiazol-2-ylbenzamide

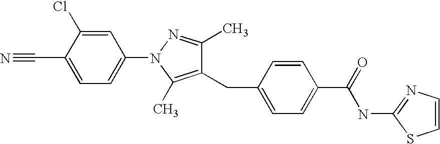

Using 2-aminothiazole as a starting material and in the same manner as in Example 266, the title compound was obtained.

MS (ESI+, m/e) 448 (M+1)

### Example 287

### 2-chloro-4-{3,5-dimethyl-4-[4-(morpholin-4-ylcarbonyl)benzyl]-1H-pyrazol-1-yl}benzonitrile

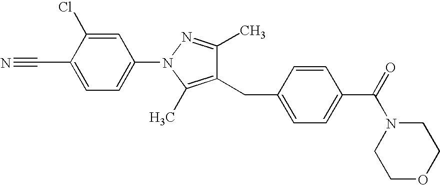

Using 4-{[1-(3-chloro-4-cyanophenyl)-3,5-dimethyl-1H-pyrazol-4-yl]methyl}benzoic acid synthesized in Example 92 and morpholine as starting materials and in the same manner as in Example 83, the title compound was obtained.

¹H-NMR (CDCl₃) δ:2.19 (3H, s), 2.34 (3H, s), 3.35-3.94 (8H, m), 3.82 (2H, s), 7.18 (2H, d), 7.34 (2H, d), 7.52 (1H, dd), 7.71-7.78 (2H, m).

### Example 288

### 4-{[1-(3-chloro-4-cyanophenyl)-3,5-dimethyl-1H-pyrazol-4-yl]methyl}-N-(2-hydroxyethyl)benzamide

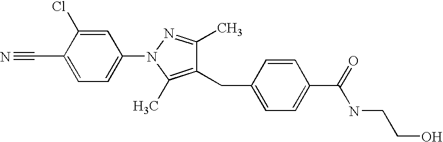

Using 4-{[1-(3-chloro-4-cyanophenyl)-3,5-dimethyl-1H-pyrazol-4-yl]methyl}benzoic acid synthesized in Example 92 and 2-aminoethanol as starting materials and in the same manner as in Example 84, the title compound was obtained.

¹H-NMR (CDCl₃) δ:2.17 (3H, s), 2.32 (3H, s), 2.56 (1H, brs), 3.58-3.67 (2H, m), 3.84 (2H, s), 3.95 (2H, d), 6.58 (1H, brs), 7.20 (2H, d), 7.51 (1H, d), 7.67-7.78 (4H, m).

### Example 289

### 2-{[1-(3-chloro-4-cyanophenyl)-3,5-dimethyl-1H-pyrazol-4-yl]methyl}benzamide

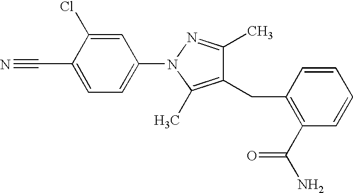

Using 2-{[1-(3-chloro-4-cyanophenyl)-3,5-dimethyl-1H-pyrazol-4-yl]methyl}benzoic acid synthesized in Example 90 as a starting material and in the same manner as in Example 75, the title compound was obtained.

¹H-NMR (DMSO-d₆) δ:2.03 (3H, s), 2.32 (3H, s), 3.94 (2H, s), 7.02 (1H, d), 7.17-7.46 (4H, m), 7.73 (1H, dd), 7.80 (1H, s), 7.93 (1H, d), 8.08 (1H, d).

### Example 290

### 3-{[1-(3-chloro-4-cyanophenyl)-3,5-dimethyl-1H-pyrazol-4-yl]methyl}benzamide

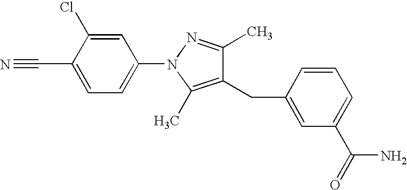

Using 3-{[l-(3-chloro-4-cyanophenyl)-3,5-dimethyl-1H-pyrazol-4-yl]methyl}benzoic acid synthesized in Example 91 as a starting material and in the same manner as in Example 75, the title compound was obtained.

¹H-NMR (DMSO-d₆) δ:2.10 (3H, s), 2.40 (3H, s), 3.83 (2H, s), 7.26-7.42 (3H, m), 7.65-7.80 (3H, m), 7.95 (2H, d), 8.08 (1H, d).

### Example 291

### 2-{[1-(3-chloro-4-cyanophenyl)-3,5-dimethyl-1H-pyrazol-4-yl]methyl}-N-methylbenzamide

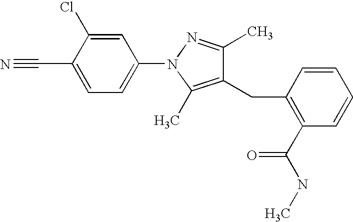

Using 2-{[1-(3-chloro-4-cyanophenyl)-3,5-dimethyl-1H-pyrazol-4-yl]methyl}benzoic acid synthesized in Example 90 and 2 mol/l methylamine-THF solution as starting materials and in the same manner as in Example 75, the title compound was obtained.

¹H-NMR (CDCl₃) δ:2.10 (3H, s), 2.31 (3H, s), 2.96 (3H, d), 4.01 (2H, s), 5.80 (1H, s), 7.05 (1H, d), 7.20-7.39 (3H, m), 7.51 (1H, dd), 7.69-7.78 (2H, m).

### Example 292

### 3-{[1-(3-chloro-4-cyanophenyl)-3,5-dimethyl-1H-pyrazol-4-yl]methyl}-N-methylbenzamide

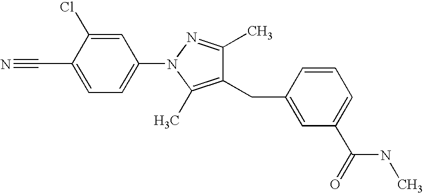

Using 3-{[1-(3-chloro-4-cyanophenyl)-3,5-dimethyl-1H-pyrazol-4-yl]methyl}benzoic acid synthesized in Example 91 and 2 mol/l methylamine-THF solution as starting materials and in the same manner as in Example 75, the title compound was obtained.

¹H-NMR (CDCl₃) δ:2.18 (3H, s), 2.33 (3H, s), 3.01 (3H, d), 3.83 (2H, s), 6.07 (1H, brs), 7.22-7.29 (1H, m), 7.34 (1H, t), 7.47-7.57 (2H, m), 7.61 (1H, s), 7.71-7.80 (2H, m).

### Example 293

### 2-{[1-(3-chloro-4-cyanophenyl)-3,5-dimethyl-1H-pyrazol-4-yl]methyl}-N,N-dimethylbenzamide

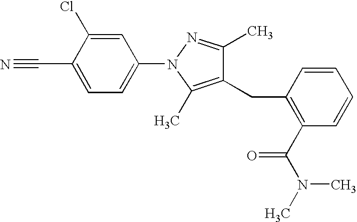

Using 2-{[1-(3-chloro-4-cyanophenyl)-3,5-dimethyl-1H-pyrazol-4-yl]methyl}benzoic acid synthesized in Example 90 and 2 mol/l dimethylamine-THF solution as starting materials and in the same manner as in Example 75, the title compound was obtained.

¹H-NMR (CDCl₃) δ:2.12 (3H, s), 2.30 (3H, s), 2.72 (3H, s), 3.04 (3H, s), 3.81 (2H, s), 7.08-7.14 (1H, m), 7.15-7.20 (1H, m), 7.22-7.33 (2H, m), 7.50 (1H, dd), 7.71-7.76 (2H, m).

### Example 294

### 3-{[1-(3-chloro-4-cyanophenyl)-3,5-dimethyl-1H-pyrazol-4-yl]methyl}-N,N-dimethylbenzamide

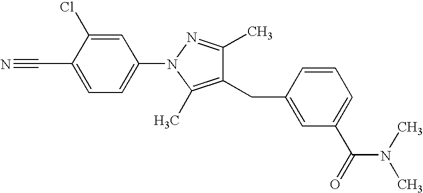

Using 3-{[1-(3-chloro-4-cyanophenyl)-3,5-dimethyl-1H-pyrazol-4-yl]methyl}benzoic acid synthesized in Example 91 and 2 mol/l dimethylamine-THF solution as starting materials and in the same manner as in Example 75, the title compound was obtained.

¹H-NMR (CDCl₃) δ:2.18 (3H, s), 2.33 (3H, s), 2.96 (3H, s), 3.10 (3H, s), 3.81 (2H, s), 7.14-7.37 (4H, m), 7.51 (1H, dd), 7.70-7.76 (2H, m).

### Example 295

### (±)-4-{[1-(3-chloro-4-cyanophenyl)-3,5-dimethyl-1H-pyrazol-4-yl]methyl}-N-(2-hydroxypropyl)benzamide

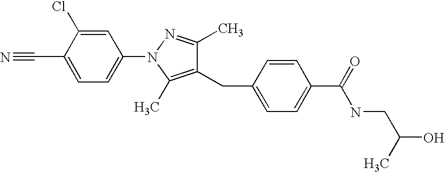

A mixture of 4-{[1-(3-chloro-4-cyanophenyl)-3,5-dimethyl-1H-pyrazol-4-yl]methyl}benzoic acid (0.100 g) synthesized in Example 92, (±)-1-amino-2-propanol (0.0421 mL), 4-(4,6-dimethoxy-1,3,5-triazin-2-yl)-4-methylmorpholin-4-ium chloride (0.151 g), THF (1.0 mL) and 2-propanol (1.0 mL) was stirred at room temperature for 21 hr. The reaction mixture was neutralized with saturated aqueous sodium hydrogen carbonate solution, and the mixture was extracted with ethyl acetate. The organic layer was washed with saturated brine, dried over anhydrous magnesium sulfate and concentrated. The obtained residue was purified by column chromatography (hexane-ethyl acetate) and recrystallized from ethyl acetate-hexane to give the title compound (0.0838 g) as a white solid.

¹H-NMR (CDCl₃) δ:1.26 (3H, d), 2.18 (3H, s), 2.32 (3H, s), 2.34 (1H, s), 3.25-3.37 (1H, m), 3.62-3.73 (1H, m), 3.84 (2H, s), 3.99-4.11 (1H, m), 6.52 (1H, brs), 7.20 (2H, d), 7.51 (1H, dd), 7.69-7.76 (4H, m).

### Example 296

### 4-{[1-(3-chloro-4-cyanophenyl)-3,5-dimethyl-1H-pyrazol-4-yl]methyl}-N-(2-hydroxyethyl)-N-methylbenzamide

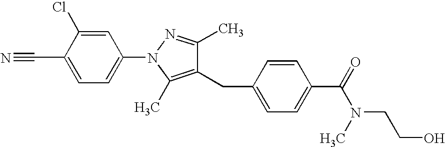

Using 4-{[1-(3-chloro-4-cyanophenyl)-3,5-dimethyl-1H-pyrazol-4-yl]methyl}benzoic acid synthesized in Example 92 and 2-(methylamino)ethanol as starting materials and in the same manner as in Example 295, the title compound was obtained.

¹H-NMR (CDCl₃) δ:2.19 (3H, s), 2.33 (3H, s), 3.07 (3H, s), 3.28 (1H, brs), 3.64-4.01 (6H, m), 7.17 (2H, d), 7.39 (2H, d), 7.52 (1H, dd), 7.71-7.77 (2H, m).

### Example 297

### 2-chloro-4-{3,5-dimethyl-4-[4-(5-methyl-1,3,4-oxadiazol-2-yl)benzyl]-1H-pyrazol-1-yl}benzonitrile

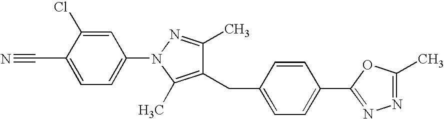

A mixture of 4-{[1-(3-chloro-4-cyanophenyl)-3,5-dimethyl-1H-pyrazol-4-yl]methyl}benzoic acid (0.128 g) obtained in Example 92, acetohydrazide (0.0311 g), N-[3-(dimethylamino)propyl]-N′-ethylcarbodiimide hydrochloride (0.0805 g), N-hydroxybenzotriazole (0.0567 g) and DMF (1.3 mL) was stirred at room temperature for 3 hr. The reaction mixture was neutralized with saturated aqueous sodium hydrogen carbonate solution, and the mixture was extracted twice with ethyl acetate. The organic layers were combined, washed with saturated brine, dried over anhydrous magnesium sulfate and concentrated to give a white solid (0.115 g). The solid was suspended in THF (5.0 mL), p-toluenesulfonyl chloride (0.100 g) and triethylamine (0.0976 mL) were added, and the mixture was heated under reflux for 15 hr. The reaction mixture was allowed to cool, saturated aqueous sodium hydrogen carbonate solution was added, and the mixture was concentrated under reduced pressure. The residue was extracted with ethyl acetate. The organic layer was washed with saturated brine, dried over anhydrous magnesium sulfate and concentrated. The obtained residue was purified by column chromatography (hexane-ethyl acetate) and further purified by reversed-phase high performance liquid chromatography (acetonitrile-water-trifluoroacetic acid) to give the title compound (0.0219 g) as a white solid.

¹H-NMR (CDCl₃) δ:2.24 (3H, s), 2.31 (3H, s), 2.67 (3H, s), 3.89 (2H, s), 7.29 (2H, d), 7.50 (1H, dd), 7.69 (1H, d), 7.79 (1H, d), 7.97 (2H, d).

### Example 298

### 4-[4-(4-fluorobenzyl)-3,5-dimethyl-1H-pyrazol-1-yl]benzene-1,2-dicarbonitrile

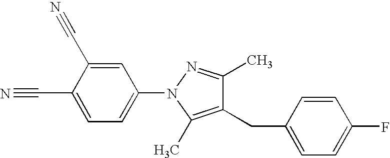

Using 4-(4-fluorobenzyl)-3,5-dimethyl-1H-pyrazole synthesized in Reference Example 34 and 4-fluorophthalonitrile as starting materials and in the same manner as in Example 46, the title compound was obtained.

¹H-NMR (CDCl₃) δ:2.18 (3H, s), 2.37 (3H, s), 3.77 (2H, s), 6.93-7.02 (2H, m), 7.04-7.13 (2H, m), 7.83-7.91 (2H, m), 8.01-8.05 (1H, m).

### Example 299

### 4-[4-(4-fluorobenzyl)-3,5-dimethyl-1H-pyrazol-1-yl]-3-(trifluoromethyl)benzonitrile

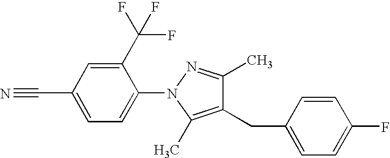

Using 4-(4-fluorobenzyl)-3,5-dimethyl-1H-pyrazole synthesized in Reference Example 34 and 3-trifluoromethyl-4-fluorobenzonitrile as starting materials and in the same manner as in Example 46, the title compound was obtained.

¹H-NMR (CDCl₃) δ:1.97 (3H, s), 2.15 (3H, s), 3.77 (2H, s), 6.93-7.12 (4H, m), 7.52 (1H, d), 7.96 (1H, d), 8.11 (1H, s).

### Example 300

### 4-{[1-(3-chloro-4-cyanophenyl)-3,5-dimethyl-1H-pyrazol-4-yl]methyl}-N-(2,2,2-trifluoroethyl)benzamide

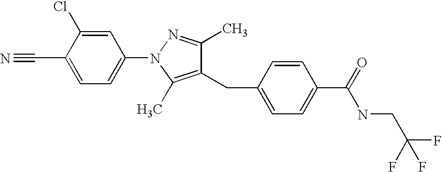

Using 4-{[1-(3-chloro-4-cyanophenyl)-3,5-dimethyl-1H-pyrazol-4-yl]methyl}benzoic acid synthesized in Example 92, 2,2,2-trifluoroethylaminehydrochloride and triethylamine as starting materials and in the same manner as in Example 295, the title compound was obtained.

¹H-NMR (CDCl₃) δ:2.18 (3H, s), 2.33 (3H, s), 3.85 (2H, s), 4.06-4.20 (2H, m), 6.28 (1H, brs), 7.19-7.29 (2H, m), 7.51 (1H, dd), 7.69-7.78 (4H, m).

### Example 301

### 2-chloro-4-{3,5-dimethyl-4-[4-(1,3,4-oxadiazol-2-yl)benzyl]-1H-pyrazol-1-yl}benzonitrile

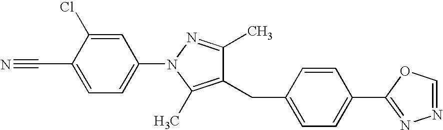

A mixture of 4-{[1-(3-chloro-4-cyanophenyl)-3,5-dimethyl-1H-pyrazol-4-yl]methyl}benzoic acid (0.100 g) synthesized in Example 92, formhydrazide (0.0328 g), 4-(4,6-dimethoxy-1,3,5-triazin-2-yl)-4-methylmorpholin-4-ium chloride (0.151 g), THF (1.0 mL) and 2-propanol (1.0 mL) was stirred at room temperature for 17 hr. The reaction mixture was concentrated under reduced pressure and neutralized with saturated aqueous sodium hydrogen carbonate solution, and the mixture was extracted twice with ethyl acetate. The organic layers were combined, washed with saturated brine, dried over anhydrous magnesium sulfate and concentrated to give an amorphous solid (0.144 g). The solid was mixed with toluene (2.0 mL), p-toluenesulfonic acid monohydrate (0.0519 g) was added, and the mixture was heated under reflux for 2 hr. The reaction mixture was allowed to cool and concentrated under reduced pressure. The residue was neutralized with saturated aqueous sodium hydrogen carbonate solution, and the mixture was extracted twice with ethyl acetate. The organic layers were combined, washed with saturated brine, dried over anhydrous magnesium sulfate and concentrated. The obtained residue was purified by column chromatography (hexane-ethyl acetate) to give the title compound (0.0237 g) as a white solid.

¹H-NMR (CDCl₃) δ:2.20 (3H, s), 2.35 (3H, s), 3.88 (2H, s), 7.27-7.33 (2H, m), 7.52 (1H, dd), 7.70-7.78 (2H, m), 8.02 (2H, d), 8.45 (1H, s).

### Example 302

### 2-chloro-4-{3,5-dimethyl-4-[4-(5-oxo-4,5-dihydro-1,3,4-oxadiazol-2-yl)benzyl]-1H-pyrazol-1-yl}benzonitrile

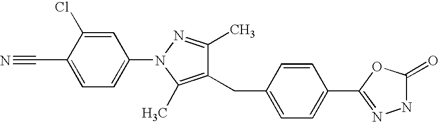

A mixture of 4-{[1-(3-chloro-4-cyanophenyl)-3,5-dimethyl-1H-pyrazol-4-yl]methyl}benzoic acid (0.300 g) obtained in Example 92, hydrazine monohydrate (0.0798 mL), 4-(4,6-dimethoxy-1,3,5-triazin-2-yl)-4-methylmorpholin-4-ium chloride (0.454 g), THF (3.0 mL) and 2-propanol (3.0 mL) was stirred at room temperature for 17 hr. The reaction mixture was concentrated under reduced pressure and neutralized with saturated aqueous sodium hydrogen carbonate solution, and the mixture was extracted twice with ethyl acetate. The organic layers were combined, washed with saturated brine, dried over anhydrous magnesium sulfate and concentrated. The residue was purified by column chromatography (hexane-ethyl acetate) to give a colorless amorphous solid (0.291 g). The amorphous solid (0.100 g) was mixed with 1,1′-carbonylbis(1H-imidazole) (0.0512 g), triethylamine (0.0733 mL) and THF (1.0 mL), and the mixture was stirred at room temperature for 20 hr. The reaction mixture was concentrated under reduced pressure, and the residue was purified by column chromatography (hexane-ethyl acetate). The resultant product was recrystallized from ethyl acetate to give the title compound (0.0355 g) as a white solid.

¹H-NMR (DMSO-d₆) δ:2.10 (3H, s), 2.40 (3H, s), 3.87 (2H, s), 7.01 (1H, brs), 7.34 (2H, d), 7.69-7.79 (3H, m), 7.95 (1H, d), 8.09 (1H, d).

### Example 303

### 4-{[1-(3-chloro-4-cyanophenyl)-3,5-dimethyl-1H-pyrazol-4-yl]methyl}-N-[(1-hydroxycyclopropyl)methyl]benzamide

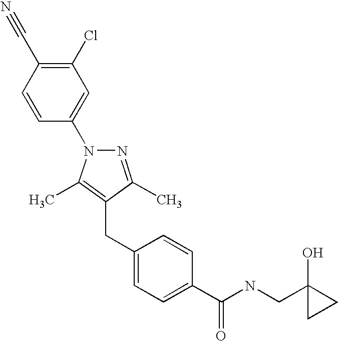

To a solution of 4-{[1-(3-chloro-4-cyanophenyl)-3,5-dimethyl-1H-pyrazol-4-yl]methyl}benzoic acid (100 mg) synthesized in Example 92 and 4-(4,6-dimethoxy-1,3,5-triazin-2-yl)-4-methylmorpholin-4-ium chloride (113 mg) in THF-2-propanol (1:1, 2 ml) was added 1-(aminomethyl)cyclopropanol (47.6 mg), and the mixture was stirred at room temperature for 21 hr. The reaction mixture was concentrated and mixed with 1 mol/l hydrochloric acid, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from ethyl acetate-hexane to give the title compound (63.7 mg) as white crystals.

¹H-NMR (CDCl₃) δ:0.62-0.70 (2H, m), 0.83-0.91 (2H, m), 2.18 (3H, s), 2.32 (3H, s), 3.11 (1H, s), 3.59 (2H, d), 3.84 (2H, s), 6.55 (1H, brs), 7.21 (2H, d), 7.51 (1H, dd), 7.68-7.80 (4H, m).

### Example 304

### 2-chloro-4-{3,5-dimethyl-4-[4-(3-methyl-1,2,4-oxadiazol-5-yl)benzyl]-1H-pyrazol-1-yl}benzonitrile

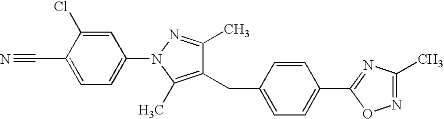

4-{[1-(3-Chloro-4-cyanophenyl)-3,5-dimethyl-1H-pyrazol-4-yl]methyl}benzoic acid (0.100 g) obtained in Example 92 was suspended in THF (1.0 mL), thionyl chloride (0.0597 mL) was added, and the mixture was stirred at room temperature for 3 hr. The reaction mixture was concentrated under reduced pressure, the residue was dissolved in THF (1.0 mL), and the mixture was ice-cooled. Acetamide oxime (0.0243 g) was added to the solution, and the mixture was stirred at room temperature for 22 hr. The reaction mixture was neutralized with saturated aqueous sodium hydrogen carbonate solution, and the mixture was extracted twice with ethyl acetate. The organic layers were combined, washed with saturated brine, dried over anhydrous magnesium sulfate and concentrated to give a colorless oil (0.116 g). The oil was mixed with xylene (1.0 mL) and heated under reflux while removing water by Dean-stark reflux condenser for 1.5 hr. The reaction mixture was allowed to cool to room temperature and concentrated under reduced pressure. The residue was purified by column chromatography (hexane-ethyl acetate), and the resultant product was recrystallized from ethyl acetate-hexane to give the title compound (0.0461 g) as a white solid.

¹H-NMR (DMSO-d₆) δ:2.11 (3H, s), 2.40 (3H, s), 2.41 (3H, s), 3.91 (2H, s), 7.42 (2H, d), 7.76 (1H, dd), 7.94-8.13 (4H, m).

### Example 305

### methyl 4-{[1-(3-chloro-4-cyanophenyl)-3,5-dimethyl-1H-pyrazol-4-yl]oxy}benzoate

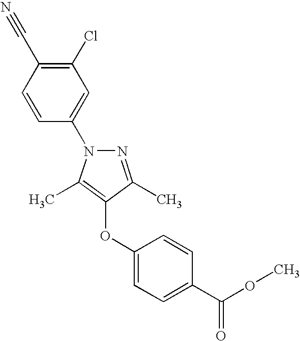

To a solution of methyl 4-[(3,5-dimethyl-1H-pyrazol-4-yl)oxy]benzoate (3.00 g) synthesized in Reference Example 16 in DMF (25 ml) was added sodium hydride (0.580 g) at 0° C., and the mixture was stirred for 30 min. 2-Chloro-4-fluorobenzonitrile (3.79 g) was added to the reaction mixture, and the mixture was stirred for 1 hr. The mixture was mixed with water and extracted with ethyl acetate. The ethyl acetate layer was washed with water and saturated brine, dried over anhydrous sodium sulfate and concentrated. The residue was purified by column chromatography (hexane-ethyl acetate) to give the title compound (4.08 g) as a white solid.

¹H-NMR (CDCl₃) δ:2.14 (3H, s), 2.32 (3H, s), 3.90 (3H, s), 6.92-6.99 (2H, m), 7.57 (1H, dd), 7.77 (1H, d), 7.80 (1H, d), 7.98-8.06 (2H, m).

### Example 306

### 4-{[l-(3-chloro-4-cyanophenyl)-3,5-dimethyl-1H-pyrazol-4-yl]oxy}benzoic acid

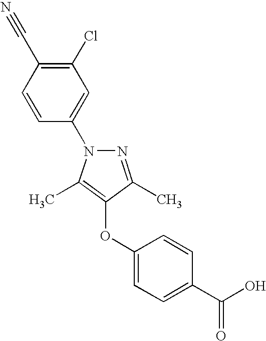

A solution of methyl 4-{[1-(3-chloro-4-cyanophenyl)-3,5-dimethyl-1H-pyrazol-4-yl]oxy}benzoate (3.87 g) synthesized in Example 305 in THF-methanol (1:1, 154 ml) was heated to 40° C., 1 mol/l aqueous sodium hydroxide solution (39 ml) was added, and the mixture was stirred for 4 hr. The reaction mixture was allowed to cool, mixed with 1 mol/l hydrochloric acid and concentrated. The residue was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) to give the title compound (3.57 g) as a white solid.

¹H-NMR (DMSO-d₆) δ:2.06 (3H, s), 2.30 (3H, s), 7.03-7.10 (2H, m), 7.81 (1H, dd), 7.90-7.97 (2H, m), 8.02 (1H, d), 8.13 (1H, d), 12.80 (1H, brs).

### Example 307

### 4-{[1-(3-chloro-4-cyanophenyl)-3,5-dimethyl-1H-pyrazol-4-yl]oxy}benzamide

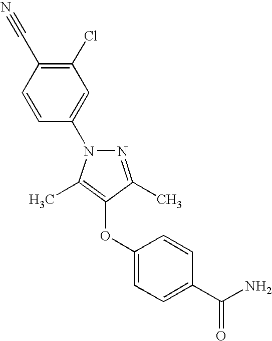

A solution of 4-{[1-(3-chloro-4-cyanophenyl)-3,5-dimethyl-1H-pyrazol-4-yl]oxy}benzoic acid (100 mg) synthesized in Example 306 and 1-hydroxybenzotriazole ammonium salt (101 mg), N-(3-dimethylaminopropyl)-N′-ethylcarbodiimide hydrochloride (78.2 mg) in DMF (1 ml) was stirred at room temperature for 2 hr. The reaction mixture was ice-cooled and mixed with 1 mol/l hydrochloric acid, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from ethyl acetate-hexane to give the title compound (64.2 mg) as white crystals.

¹H-NMR (DMSO-d₆) δ:2.06 (3H, s), 2.30 (3H, s), 6.97-7.05 (2H, m), 7.27 (1H, brs), 7.81 (1H, dd), 7.84-7.93 (3H, m), 8.01 (1H, d), 8.13 (1H, d).

### Example 308

### 4-{4-[(4-bromo-2-fluorophenoxy)methyl]-3,5-dimethyl-1H-pyrazol-1-yl}benzonitrile

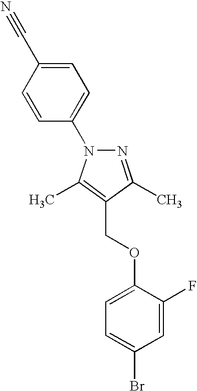

To a solution of 4-[4-(hydroxymethyl)-3,5-dimethyl-1H-pyrazol-1-yl]benzonitrile (100 mg) synthesized in Reference Example 21 in THF (5 ml) were added 1,1′-(azodicarbonyl)dipiperidine (222 mg), 4-bromo-2-fluorophenol (72.3 μL) and n-tributylphosphine (219 μL), and the mixture was stirred at room temperature for 17 hr. The reaction mixture was ice-cooled and mixed with saturated aqueous ammonium chloride solution, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from ethyl acetate-hexane to give the title compound (88.4 mg) as white crystals.

¹H-NMR (CDCl₃) δ:2.34 (3H, s), 2.39 (3H, s), 4.94 (2H, s), 6.96 (1H, t), 7.19-7.30 (2H, m), 7.57-7.63 (2H, m), 7.73-7.80 (2H, m).

### Example 309

### 2-chloro-4-(3,5-dimethyl-4-{4-[5-(trifluoromethyl)-1,3,4-oxadiazol-2-yl]benzyl}-1H-pyrazol-1-yl)benzonitrile

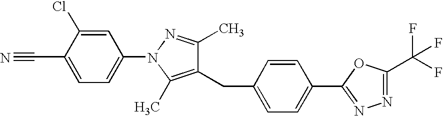

### Step 1

A mixture of 4-{[1-(3-chloro-4-cyanophenyl)-3,5-dimethyl-1H-pyrazol-4-yl]methyl}benzoic acid (0.500 g) obtained in Example 92, tert-butyl carbazate (0.361 g), 4-(4,6-dimethoxy-1,3,5-triazin-2-yl)-4-methylmorpholin-4-ium chloride (0.755 g), THF (10.0 mL) and 2-propanol (10.0 mL) was stirred at room temperature for 15 hr. The reaction mixture was concentrated under reduced pressure and neutralized with saturated aqueous sodium hydrogen carbonate solution, and the mixture was extracted twice with ethyl acetate. The organic layers were combined, washed with saturated brine, dried over anhydrous magnesium sulfate and concentrated. The residue was purified by column chromatography (hexane-ethyl acetate) to give a white solid (0.539 g). The obtained solid was suspended in 4 mol/l hydrogen chloride-ethyl acetate solution (10 mL), and the suspension was stirred at room temperature for 2 hr. The reaction mixture was concentrated under reduced pressure to give 4-{[1-(3-chloro-4-cyanophenyl)-3,5-dimethyl-1H-pyrazole-4-yl]methyl}benzohydrazide hydrochloride (0.595 g) as a white solid.

### Step 2

4-{[1-(3-Chloro-4-cyanophenyl)-3,5-dimethyl-1H-pyrazol-4-yl]methyl}benzohydrazide hydrochloride (0.195 g) obtained in step 1 was suspended in THF (2.0 mL), triethylamine (0.153 mL) and trifluoroacetic anhydride (0.0765 mL) were added, and the mixture was stirred at room temperature for 17 hr. Triethylamine (0.153 mL) and trifluoroacetic anhydride (0.0765 mL) were further added to the reaction mixture, and the mixture was stirred at room temperature for 7 hr. The reaction mixture was concentrated under reduced pressure and neutralized with saturated aqueous sodium hydrogen carbonate solution, and the mixture was extracted twice with ethyl acetate. The organic layers were combined, washed with saturated brine, dried over anhydrous magnesium sulfate and concentrated to give a colorless amorphous solid (0.201 g). The solid was suspended in toluene (2.0 mL), p-toluenesulfonic acid monohydrate (0.0698 g) was added, and the mixture was heated under reflux for 24 hr. The reaction mixture was allowed to cool and concentrated under reduced pressure. The ⅘ of the residue was suspended in toluene (7.0 mL), triethylamine (0.124 mL) and p-toluenesulfonyl chloride (0.112 g) were added, and the mixture was heated at 150° C. for 20 min under microwave irradiation. The reaction mixture was allowed to cool and concentrated under reduced pressure. The residue was neutralized with saturated aqueous sodium hydrogen carbonate solution, and the mixture was extracted with ethyl acetate. The organic layer was washed with saturated brine, dried over anhydrous magnesium sulfate and concentrated. The obtained residue was purified by column chromatography (hexane-ethyl acetate) and recrystallized from ethyl acetate-hexane to give the title compound (0.060 g) as a white solid.

¹H-NMR (CDCl₃) δ:2.19 (3H, s), 2.35 (3H, s), 3.89 (2H, s), 7.33 (2H, d), 7.52 (1H, dd), 7.75 (2H, dd), 8.04 (2H, d).

### Example 310

### 2-chloro-4-{4-[4-(5-cyclopropyl-1,3,4-oxadiazol-2-yl)benzyl]-3,5-dimethyl-1H-pyrazol-1-yl}benzonitrile

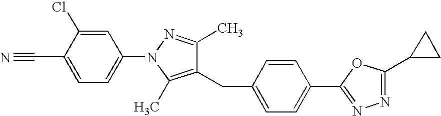

A mixture of 4-{[1-(3-chloro-4-cyanophenyl)-3,5-dimethyl-1H-pyrazol-4-yl]methyl}benzohydrazide hydrochloride (0.150 g) obtained in Example 309, step 1, triethylamine (0.059 mL), cyclopropanecarboxylic acid (0.0337 mL), 4-(4,6-dimethoxy-1,3,5-triazin-2-yl)-4-methylmorpholin-4-ium chloride (0.117 g), THF (2.0 mL) and 2-propanol (2.0 mL) was stirred at room temperature for 1 day. Cyclopropanecarboxylic acid (0.0337 mL) and triethylamine (0.059 mL) were added to the reaction mixture, and the mixture was further stirred at room temperature for 3 days. The reaction mixture was concentrated under reduced pressure and neutralized with 1 mol/l hydrochloric acid, and the mixture was extracted with ethyl acetate. The organic layer was washed with saturated aqueous sodium hydrogen carbonate solution and saturated brine, dried over anhydrous magnesium sulfate and concentrated. The obtained residue was suspended in toluene (2.0 mL), p-toluenesulfonic acid monohydrate (0.0536 g) was added, and the mixture was heated at 150° C. for 20 min, at 170° C. for 20 min and at 180° C. for 20 min in a tightly-sealed container under microwave irradiation. The reaction mixture was allowed to cool and concentrated under reduced pressure. The residue was neutralized with saturated aqueous sodium hydrogen carbonate solution, and the mixture was extracted twice with ethyl acetate. The organic layers were combined, washed with saturated brine, dried over anhydrous magnesium sulfate and concentrated. The obtained residue was purified twice by column chromatography (hexane-ethyl acetate) to give the title compound (0.0237 g) as a white solid.

¹H-NMR (CDCl₃) δ:1.15-1.23 (4H, m), 2.15-2.30 (4H, m), 2.34 (3H, s), 3.85 (2H, s), 7.22-7.29 (2H, m), 7.52 (1H, dd), 7.71-7.78 (2H, m), 7.89-7.96 (2H, m).

### Example 311

### 4-{[1-(3-chloro-4-cyanophenyl)-3,5-dimethyl-1H-pyrazol-4-yl]methyl}-N-(3-hydroxypropyl)benzamide

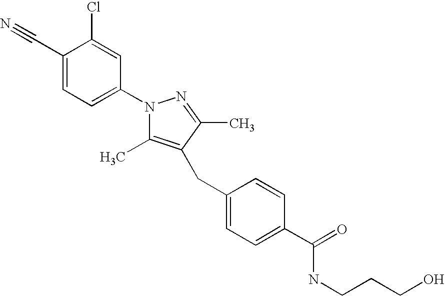

Using 4-{[1-(3-chloro-4-cyanophenyl)-3,5-dimethyl-1H-pyrazol-4-yl]methyl}benzoic acid synthesized in Example 92 and 3-amino-1-propanol as starting materials and in the same manner as in Example 295, the title compound was obtained.

¹H-NMR (CDCl₃) δ:1.75-1.86 (2H, m), 2.18 (3H, s), 2.32 (3H, s), 2.96 (1H, brs), 3.64 (2H, q), 3.72 (2H, q), 3.84 (2H, s), 6.55 (1H, brs), 7.16-7.23 (2H, m), 7.51 (1H, dd), 7.66-7.78 (4H, m).

### Example 312

### 4-{[1-(3-chloro-4-cyanophenyl)-3,5-dimethyl-1H-pyrazol-4-yl]methyl}-N-[2-(2-hydroxyethoxy)ethyl]benzamide

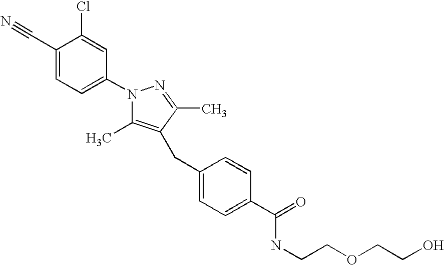

Using 4-{[1-(3-chloro-4-cyanophenyl)-3,5-dimethyl-1H-pyrazol-4-yl]methyl}benzoic acid synthesized in Example 92 and 2-(2-aminoethoxy)ethanol as starting materials and in the same manner as in Example 295, the title compound was obtained.

¹H-NMR (CDCl₃) δ:1.94 (1H, t), 2.18 (3H, s), 2.32 (3H, s), 3.59-3.65 (2H, m), 3.68 (4H, d), 3.73-3.81 (2H, m), 3.83 (2H, s), 6.55 (1H, brs), 7.20 (2H, d), 7.51 (1H, dd), 7.68-7.79 (4H, m).

### Example 313

### 4-{[l-(3-chloro-4-cyanophenyl)-3,5-dimethyl-1H-pyrazol-4-yl]methyl}-N′-(4,6-dimethoxy-1,3,5-triazin-2-yl)benzohydrazide

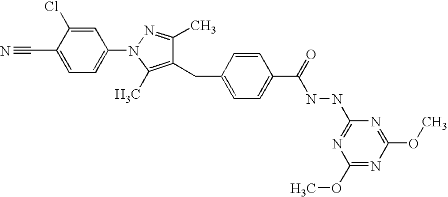

Recrystallization of 4-{[1-(3-chloro-4-cyanophenyl)-3,5-dimethyl-1H-pyrazol-4-yl]methyl}-N′-(4,6-dimethoxy-1,3,5-triazin-2-yl)benzohydrazide (0.0677 g) obtained in Example 320, step 1 from ethyl acetate-hexane gave the title compound (0.0617 g).

¹H-NMR (CDCl₃) δ:2.18 (3H, s), 2.33 (3H, s), 3.84 (2H, s), 3.96 (6H, s), 7.20 (2H, d), 7.49-7.56 (2H, m), 7.71-7.80 (4H, m), 8.54 (1H, brs).

### Example 314

### 2-chloro-4-[4-(4-cyanophenoxy)-3,5-dimethyl-1H-pyrazol-1-yl]benzonitrile

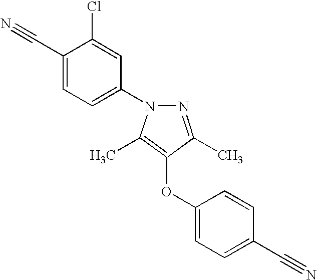

To a solution of 4-{[1-(3-chloro-4-cyanophenyl)-3,5-dimethyl-1H-pyrazol-4-yl]oxy}benzamide (181 mg) synthesized in Example 307 and pyridine (252 μL) in DMF (1 ml) was added oxalyl dichloride (172 μL) at 0° C., and the mixture was stirred for 3 hr. The reaction solution was neutralized with saturated aqueous sodium hydrogen carbonate, and the mixture was extracted with ethyl acetate. The ethyl acetate layer was washed with saturated aqueous sodium hydrogen carbonate and saturated brine, dried over anhydrous sodium sulfate and concentrated. The residue was purified by column chromatography (hexane-ethyl acetate) and recrystallized from ethyl acetate-hexane to give the title compound (90.7 mg) as white crystals.

¹H-NMR (CDCl₃) δ:2.14 (3H, s), 2.31 (3H, s), 6.98-7.04 (2H, m), 7.56 (1H, dd), 7.60-7.66 (2H, m), 7.75-7.80 (2H, m).

### Example 315

### 4-{3,5-dimethyl-4-[(pyridin-3-yloxy)methyl]-1H-pyrazol-1-yl}benzonitrile

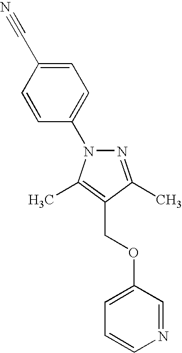

Using 4-[4-(hydroxymethyl)-3,5-dimethyl-1H-pyrazol-1-yl]benzonitrile synthesized in Reference Example 21 and pyridin-3-ol as starting materials and in the same manner as in Example 308, the title compound was obtained.

¹H-NMR (CDCl₃) δ:2.35 (3H, s), 2.41 (3H, s), 4.95 (2H, s), 7.23-7.31 (2H, m), 7.58-7.65 (2H, m), 7.74-7.80 (2H, m), 8.28 (1H, dd), 8.41 (1H, d).

### Example 316

### 4-{[1-(3-chloro-4-cyanophenyl)-3,5-dimethyl-1H-pyrazol-4-yl]oxy}-N-methylbenzamide

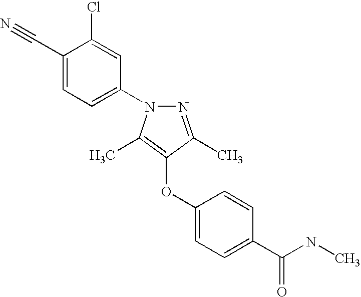

A solution of 4-{[1-(3-chloro-4-cyanophenyl)-3,5-dimethyl-1H-pyrazol-4-yl]oxy}benzoic acid (100 mg) synthesized in Example 306, 1-hydroxybenzotriazole (55.1 mg), methylamine 40% methanol solution (41.9 μL) and N-(3-dimethylaminopropyl)-N′-ethylcarbodiimide hydrochloride (78.2 mg) in DMF (1 ml) was stirred at room temperature for 3 hr. The reaction mixture was ice-cooled and mixed with 1 mol/l hydrochloric acid, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from ethyl acetate-hexane to give the title compound (50.3 mg) as white crystals.

¹H-NMR (CDCl₃) δ:2.13 (3H, s), 2.31 (3H, s), 3.02 (3H, d), 6.02 (1H, brs), 6.92-6.98 (2H, m), 7.56 (1H, dd), 7.71-7.78 (3H, m), 7.80 (1H, d).

### Example 317

### 4-{[1-(3-chloro-4-cyanophenyl)-3,5-dimethyl-1H-pyrazol-4-yl]oxy}-N,N-dimethylbenzamide

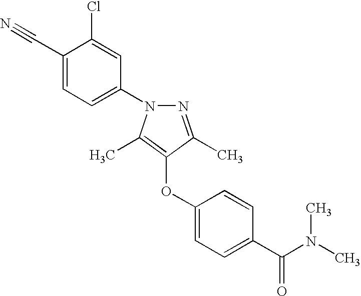

Using 4-{[1-(3-chloro-4-cyanophenyl)-3,5-dimethyl-1H-pyrazol-4-yl]oxy}benzoic acid synthesized in Example 306 and dimethylamine 2 mol/l THF solution as starting materials and in the same manner as in Example 316, the title compound was obtained.

¹H-NMR (CDCl₃) δ:2.15 (3H, s), 2.31 (3H, s), 3.06 (6H, brs), 6.90-6.96 (2H, m), 7.38-7.44 (2H, m), 7.56 (1H, dd), 7.76 (1H, d), 7.80 (1H, d).

### Example 318

### 4-{[1-(3-chloro-4-cyanophenyl)-3,5-dimethyl-1H-pyrazol-4-yl]methyl}-N-(2-methoxyethyl)benzamide

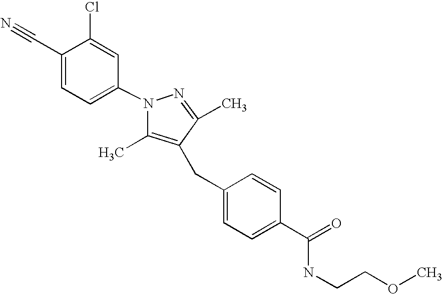

Using 4-{[l-(3-chloro-4-cyanophenyl)-3,5-dimethyl-1H-pyrazol-4-yl]methyl}benzoic acid synthesized in Example 92 and 2-methoxyethaneamine as starting materials and in the same manner as in Example 295, the title compound was obtained.

¹H-NMR (CDCl₃) δ:2.18 (3H, s), 2.32 (3H, s), 3.38 (3H, s), 3.52-3.59 (2H, m), 3.65 (2H, q), 3.83 (2H, s), 6.47 (1H, brs), 7.19 (2H, d), 7.51 (1H, dd), 7.68-7.78 (4H, m).

### Example 319

### 2-chloro-4-{4-[(4-chloro-2-fluorophenoxy)methyl]-3,5-dimethyl-1H-pyrazol-1-yl}benzonitrile

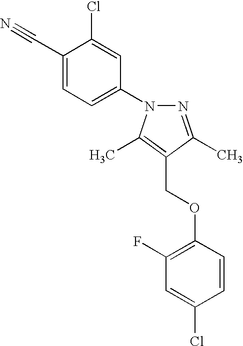

Using 2-chloro-4-[4-(hydroxymethyl)-3,5-dimethyl-1H-pyrazol-1-yl]benzonitrile synthesized in Reference Example 21 and 4-chloro-2-fluorophenol as starting materials and in the same manner as in Example 308, the title compound was obtained.

¹H-NMR (CDCl₃) δ:2.33 (3H, s), 2.42 (3H, s), 4.93 (2H, s), 7.00 (1H, t), 7.05-7.10 (1H, m), 7.13 (1H, dd), 7.50 (1H, dd), 7.72 (1H, d), 7.75 (1H, d).

### Example 320

### 4-{4-[4-(5-tert-butyl-1,3,4-oxadiazol-2-yl)benzyl]-3,5-dimethyl-1H-pyrazol-1-yl}-2-chlorobenzonitrile

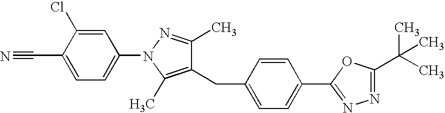

### Step 1

A mixture of 4-{[1-(3-chloro-4-cyanophenyl)-3,5-dimethyl-1H-pyrazol-4-yl]methyl}benzohydrazide hydrochloride (0.150 g) obtained in Example 309, step 1, triethylamine (0.0590 mL), pivalic acid (0.0432 g), 4-(4,6-dimethoxy-1,3,5-triazin-2-yl)-4-methylmorpholin-4-ium chloride (0.117 g), THF (2.0 mL) and 2-propanol (2.0 mL) was stirred at room temperature for 1 day. Pivalic acid (0.0432 g) and triethylamine (0.0590 mL) were added to the reaction mixture, and the mixture was further stirred at room temperature for 4 days. The reaction mixture was concentrated under reduced pressure and neutralized with 1 mol/l hydrochloric acid, and the mixture was extracted with ethyl acetate. The organic layer was washed with saturated sodium hydrogen carbonate and saturated brine, dried over anhydrous magnesium sulfate and concentrated. The obtained residue was purified by column chromatography (hexane-ethyl acetate) to give 4-{[1-(3-chloro-4-cyanophenyl)-3,5-dimethyl-1H-pyrazol-4-yl]methyl}-N′-(2,2-dimethylpropanoyl)benzohydrazide (0.0668 g) and 4-{[l-(3-chloro-4-cyanophenyl)-3,5-dimethyl-1H-pyrazol-4-yl]methyl}-N′-(4,6-dimethoxy-1,3,5-triazin-2-yl)benzohydrazide (0.0677 g).

### Step 2

A mixture of 4-{[1-(3-chloro-4-cyanophenyl)-3,5-dimethyl-1H-pyrazol-4-yl]methyl}-N′-(2,2-dimethylpropanoyl)benzohydrazide (0.0668 g) obtained in step 1, p-toluenesulfonic acid monohydrate (0.0274 g) and toluene (4.0 mL) was heated at 180° C. for 30 min in a tightly sealed container under microwave irradiation. The reaction mixture was allowed to cool and concentrated under reduced pressure. The residue was neutralized with saturated aqueous sodium hydrogen carbonate solution, and the mixture was extracted with ethyl acetate. The organic layer was washed with saturated brine, dried over anhydrous magnesium sulfate and concentrated. The obtained residue was purified by column chromatography (hexane-ethyl acetate) and recrystallized from diisopropyl ether to give the title compound (0.0373 g) as a white solid.

¹H-NMR (CDCl₃) δ:1.48 (9H, s), 2.20 (3H, s), 2.34 (3H, s), 3.86 (2H, s), 7.22-7.32 (2H, m), 7.49-7.56 (1H, m), 7.71-7.78 (2H, m), 7.92-8.01 (2H, m).

### Example 321

### (±)-4-{[1-(3-chloro-4-cyanophenyl)-3,5-dimethyl-1H-pyrazol-4-yl]methyl}-N-(2,3-dihydroxypropyl)benzamide

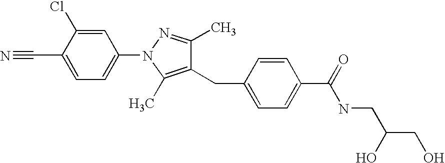

Using 4-{[1-(3-chloro-4-cyanophenyl)-3,5-dimethyl-1H-pyrazol-4-yl]methyl}benzoic acid synthesized in Example 92 and (±)-3-aminopropane-1,2-diol as starting materials and in the same manner as in Example 295, the title compound was obtained.

¹H-NMR (CDCl₃) δ:2.18 (3H, s), 2.33 (3H, s), 2.81 (1H, t), 2.87 (1H, d), 3.56-3.69 (4H, m), 3.84 (2H, s), 3.85-3.94 (1H, m), 6.55 (1H, brs), 7.21 (2H, d), 7.51 (1H, dd), 7.69-7.77 (4H, m).

### Example 322

### 4-{[1-(3-chloro-4-cyanophenyl)-3,5-dimethyl-1H-pyrazol-4-yl]methyl}-N-(1-methylethyl)benzamide

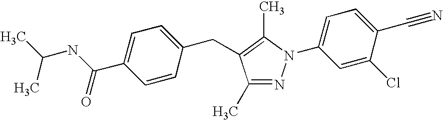

Using 4-{[1-(3-chloro-4-cyanophenyl)-3,5-dimethyl-1H-pyrazol-4-yl]methyl}benzoic acid synthesized in Example 92 and isopropylamine as starting materials and in the same manner as in Example 295, the title compound was obtained.

¹H-NMR (CDCl₃) δ:1.26 (6H, dd), 2.18 (3H, d), 2.32 (3H, d), 3.83 (2H, s), 4.21-4.36 (1H, m), 5.85 (1H, brs), 7.19 (2H, d), 7.46-7.56 (1H, m), 7.64-7.71 (1H, m), 7.71-7.80 (3H, m).

### Example 323

### 2-chloro-4-{4-[(3,4-dichlorophenoxy)methyl]-3,5-dimethyl-1H-pyrazol-1-yl}benzonitrile

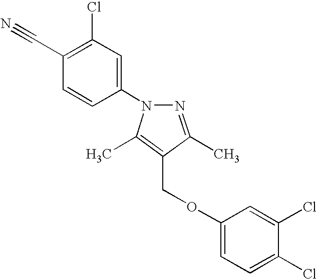

Using 2-chloro-4-[4-(hydroxymethyl)-3,5-dimethyl-1H-pyrazol-1-yl]benzonitrile synthesized in Reference Example 21 and 3,4-dichlorophenol as starting materials, the reaction of Example 308 was performed using toluene as a solvent to give the title compound.

¹H-NMR (CDCl₃) δ:2.33 (3H, s), 2.43 (3H, s), 4.85 (2H, s), 6.84 (1H, dd), 7.09 (1H, d), 7.36 (1H, d), 7.51 (1H, dd), 7.73 (1H, d), 7.76 (1H, d).

### Example 324

### 4-{4-[(4-bromophenoxy)methyl]-3,5-dimethyl-1H-pyrazol-1-yl}-2-chlorobenzonitrile

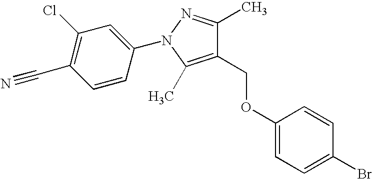

Using 4-bromophenol as a starting material and in the same manner as in Example 142, the title compound was obtained.

MS (ESI+, m/e) 416(M+1).

### Example 325

### 5-(4-{[1-(3-chloro-4-cyanophenyl)-3,5-dimethyl-1H-pyrazol-4-yl]methyl}phenyl)-1,3,4-oxadiazole-2-carbonitrile

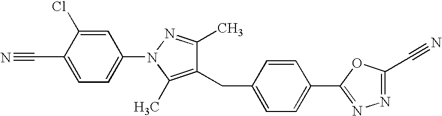

### Step 1

A mixture of 4-{[1-(3-chloro-4-cyanophenyl)-3,5-dimethyl-1H-pyrazol-4-yl]methyl}benzoic acid (0.200 g) obtained in Example 92, oxamic acid hydrazide (0.113 g), 4-(4,6-dimethoxy-1,3,5-triazin-2-yl)-4-methylmorpholin-4-ium chloride (0.302 g), THF (2.0 mL) and 2-propanol (2.0 mL) was stirred at room temperature for 3 days. THF (20 mL), isopropanol (10 mL) and DMF (5 mL) were added to the reaction mixture, and the mixture was further stirred at room temperature for 5 days. The reaction mixture was concentrated under reduced pressure and neutralized with 1 mol/l hydrochloric acid, and the mixture was extracted with ethyl acetate. The organic layer was washed with saturated aqueous sodium hydrogen carbonate solution and saturated brine, dried over anhydrous magnesium sulfate and concentrated to give 2-{2-[(4-{[1-(3-chloro-4-cyanophenyl)-3,5-dimethyl-1H-pyrazol-4-yl]methyl}phenyl)carbonyl]hydrazino}-2-oxoacetamide (0.268 g) as a white solid.

### Step 2

2-{2-[(4-{[l-(3-Chloro-4-cyanophenyl)-3,5-dimethyl-1H-pyrazol-4-yl]methyl}phenyl)carbonyl]hydrazino}-2-oxoacetamide (0.268 g) obtained in step 1 was mixed with phosphoryl chloride (3.41 g), and the mixture was heated at 150° C. for 10 min under microwave irradiation. The reaction mixture was ice-cooled and neutralized with saturated aqueous sodium hydrogen carbonate solution, and the mixture was extracted twice with ethyl acetate. The organic layers were combined, washed with saturated brine, dried over anhydrous magnesium sulfate and concentrated. The obtained residue was purified 3 times by column chromatography (hexane-ethyl acetate) and further purified by reversed-phase high performance liquid chromatography (acetonitrile-water-trifluoroacetic acid) to give the title compound (0.0087 g) as a white solid.

¹H-NMR (CDCl₃) δ:2.19 (3H, s), 2.35 (3H, s), 3.90 (2H, s), 7.34 (2H, d), 7.52 (1H, dd), 7.75 (2H, dd), 8.04 (2H, d).

### Example 326

### 2-chloro-4-(3,5-dimethyl-4-{4-[3-(1-methylethyl)-1,2,4-oxadiazol-5-yl]benzyl}-1H-pyrazol-1-yl)benzonitrile

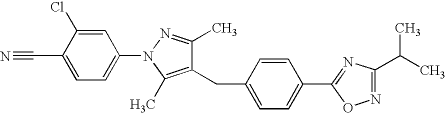

4-{[1-(3-Chloro-4-cyanophenyl)-3,5-dimethyl-1H-pyrazol-4-yl]methyl}benzoic acid (0.100 g) synthesized in Example 92 was suspended in THF (1.0 mL), thionyl chloride (0.0598 mL) was added, and the mixture was stirred at room temperature for 5 hr. The reaction mixture was concentrated under reduced pressure, and the residue was dissolved in THF (1.0 mL) and ice-cooled. N′-Hydroxy-2-methylpropanimidamide (0.0335 g) was added to the solution, and the mixture was stirred at room temperature for 22 hr. N′-Hydroxy-2-methylpropanimidamide (0.0335 g) was further added to the reaction mixture, and the mixture was stirred at room temperature for 9 hr. The reaction mixture was neutralized with saturated aqueous sodium hydrogen carbonate solution, and the mixture was extracted twice with ethyl acetate. The organic layers were combined, washed with saturated brine, dried over anhydrous magnesium sulfate and concentrated to give a white solid (0.140 g). The solid was mixed with xylene (1.0 mL), and the mixture was heated at 150° C. for 20 min in a tightly sealed container under microwave irradiation. The reaction mixture was allowed to cool to room temperature and concentrated under reduced pressure. The residue was neutralized with saturated aqueous sodium hydrogen carbonate solution, and the mixture was extracted with ethyl acetate. The organic layer was washed with saturated brine, dried over anhydrous magnesium sulfate and concentrated. The residue was purified by column chromatography (hexane-ethyl acetate), and the resultant product was recrystallized from ethyl acetate-hexane to give the title compound (0.0366 g) as a white solid.

¹H-NMR (CDCl₃) δ:1.38 (3H, s), 1.41 (3H, s), 2.19 (3H, s), 2.34 (3H, s), 3.09-3.25 (1H, m), 3.87 (2H, s), 7.25-7.32 (2H, m), 7.52 (1H, dd), 7.71-7.78 (2H, m), 8.05 (2H, d).

### Example 327

### 2-chloro-4-{3,5-dimethyl-4-[4-(3-propyl-1,2,4-oxadiazol-5-yl)benzyl]-1H-pyrazol-1-yl}benzonitrile

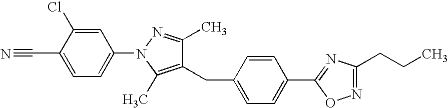

Using 4-{[1-(3-chloro-4-cyanophenyl)-3,5-dimethyl-1H-pyrazol-4-yl]methyl}benzoic acid synthesized in Example 92 and N′-hydroxybutanimidamide as starting materials and in the same manner as in Example 304, the title compound was obtained.

¹H-NMR (CDCl₃) δ:1.03 (3H, t), 1.78-1.89 (2H, m), 2.19 (3H, s), 2.34 (3H, s), 2.77 (2H, t), 3.87 (2H, s), 7.23-7.32 (2H, m), 7.52 (1H, d), 7.71-7.78 (2H, m), 8.04 (2H, d).

### Example 328

### 2-chloro-4-{3,5-dimethyl-4-[(pyridin-3-yloxy)methyl]-1H-pyrazol-1-yl}benzonitrile

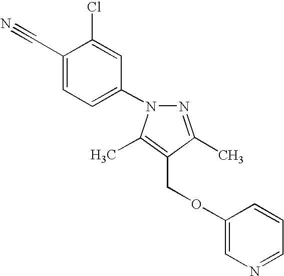

Using 2-chloro-4-[4-(hydroxymethyl)-3,5-dimethyl-1H-pyrazol-1-yl]benzonitrile synthesized in Reference Example 21 and pyridin-3-ol as starting materials, the reaction of Example 308 was performed using toluene as a solvent to give the title compound.

¹H-NMR (CDCl₃) δ:2.34 (3H, s), 2.44 (3H, s), 4.94 (2H, s), 7.25-7.30 (2H, m), 7.51 (1H, dd), 7.73 (1H, d), 7.76 (1H, d), 8.29 (1H, dd), 8.40 (1H, d).

### Example 329

### N-tert-butyl-4-{[1-(3-chloro-4-cyanophenyl)-3,5-dimethyl-1H-pyrazol-4-yl]oxy}benzamide

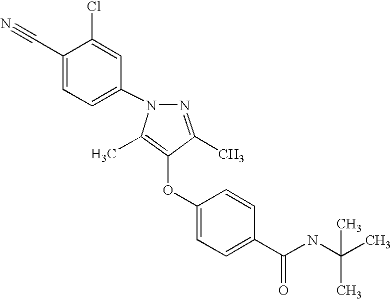

Using 4-{[1-(3-chloro-4-cyanophenyl)-3,5-dimethyl-1H-pyrazol-4-yl]oxy}benzoic acid synthesized in Example 306 and tert-butylamine as starting materials and in the same manner as in Example 316, the title compound was obtained.

¹H-NMR (CDCl₃) δ:1.47 (9H, s), 2.13 (3H, s), 2.31 (3H, s), 5.85 (1H, brs), 6.90-6.97 (2H, m), 7.56 (1H, dd), 7.67-7.73 (2H, m), 7.76 (1H, d), 7.80 (1H, d).

### Example 330

### 4-{[1-(3-chloro-4-cyanophenyl)-3,5-dimethyl-1H-pyrazol-4-yl]oxy}-N-(2-hydroxyethyl)benzamide

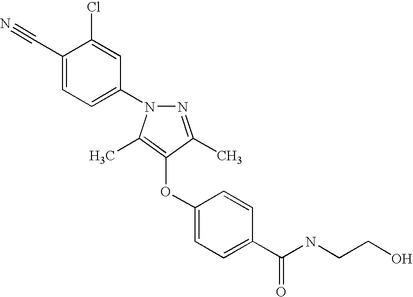

Using 4-{[1-(3-chloro-4-cyanophenyl)-3,5-dimethyl-1H-pyrazol-4-yl]oxy}benzoic acid synthesized in Example 306 and 2-aminoethanol as starting materials and in the same manner as in Example 303, the title compound was obtained.

¹H-NMR (CDCl₃) δ:2.13 (3H, s), 2.32 (3H, s), 2.44 (1H, brs), 3.60-3.68 (2H, m), 3.81-3.89 (2H, m), 6.51 (1H, brs), 6.93-7.00 (2H, m), 7.57 (1H, dd), 7.74-7.82 (4H, m).

### Example 331

### 4-{[1-(3-chloro-4-cyanophenyl)-3,5-dimethyl-1H-pyrazol-4-yl]methyl}-N-(pyridin-2-ylmethyl)benzamide

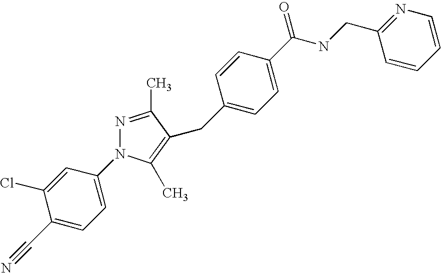

Using 4-{[1-(3-chloro-4-cyanophenyl)-3,5-dimethyl-1H-pyrazol-4-yl]methyl}benzoic acid synthesized in Example 92 and 2-(aminomethyl)pyridine as starting materials and in the same manner as in Example 83, the title compound was obtained.

¹H-NMR (CDCl₃) δ:2.19 (3H, s), 2.33 (3H, s), 3.84 (2H, s), 4.76 (2H, d), 7.19-7.28 (3H, m), 7.33 (1H, d), 7.47-7.61 (2H, m), 7.65-7.76 (3H, m), 7.81 (2H, d), 8.56 (1H, d).

### Example 332

### 2-chloro-4-[4-(4-{[(3S)-3-hydroxypyrrolidin-1-yl]carbonyl}benzyl)-3,5-dimethyl-1H-pyrazol-1-yl]benzonitrile

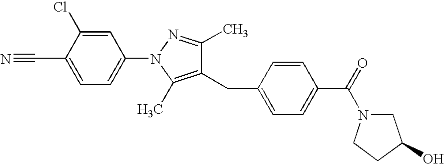

Using 4-{[1-(3-chloro-4-cyanophenyl)-3,5-dimethyl-1H-pyrazol-4-yl]methyl}benzoic acid synthesized in Example 92 and (3S)-3-hydroxypyrrolidine as starting materials and in the same manner as in Example 295, the title compound was obtained.

¹H-NMR (CDCl₃) δ:1.93-2.01 (2H, m), 2.19 (3H, s), 2.33 (3H, s), 3.37-3.84 (7H, m), 4.39-4.66 (1H, m), 7.16 (2H, d), 7.42-7.56 (3H, m), 7.70-7.77 (2H, m).

### Example 333

### 2-chloro-4-(4-{4-[(3-hydroxyazetidin-1-yl)carbonyl]phenoxy}-3,5-dimethyl-1H-pyrazol-1-yl)benzonitrile

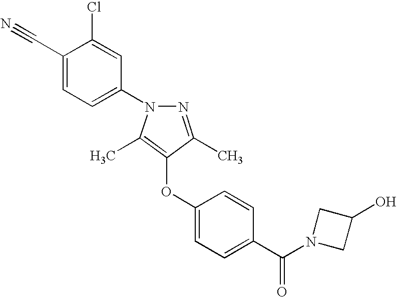

The title compound was obtained using 4-{[1-(3-chloro-4-cyanophenyl)-3,5-dimethyl-1H-pyrazol-4-yl]oxy}benzoic acid synthesized in Example 306 and 3-hydroxyazetidine hydrochloride as starting materials, in the same manner as in Example 339, and using acetone-hexane as recrystallization solvent.

¹H-NMR (CDCl₃) δ:2.14 (3H, s), 2.17 (1H, d), 2.31 (3H, s), 4.13 (2H, brs), 4.44-4.53 (2H, m), 4.68-4.79 (1H, m), 6.90-6.97 (2H, m), 7.56 (1H, dd), 7.59-7.66 (2H, m), 7.76 (1H, d), 7.79 (1H, d).

### Example 334

### 4-{[1-(3-chloro-4-cyanophenyl)-3,5-dimethyl-1H-pyrazol-4-yl]methyl}-N-(pyridin-3-ylmethyl)benzamide

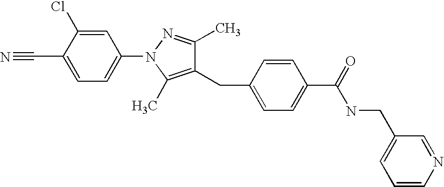

Using 4-{[1-(3-chloro-4-cyanophenyl)-3,5-dimethyl-1H-pyrazol-4-yl]methyl}benzoic acid synthesized in Example 92 and 3-(aminomethyl)pyridine as starting materials and in the same manner as in Example 83, the title compound was obtained.

¹H-NMR (CDCl₃) δ:2.17 (3H, s), 2.32 (3H, s), 3.84 (2H, s), 4.67 (2H, d), 6.38-6.48 (1H, m), 7.17-7.33 (4H, m), 7.51 (1H, dd), 7.65-7.79 (5H, m), 8.61 (1H, s).

### Example 335

### 4-{[1-(3-chloro-4-cyanophenyl)-3,5-dimethyl-1H-pyrazol-4-yl]methyl}-N-(pyridin-4-ylmethyl)benzamide

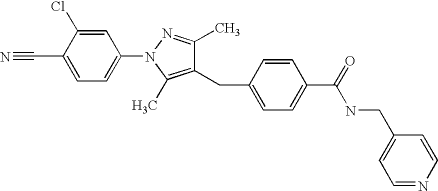

Using 4-{[1-(3-chloro-4-cyanophenyl)-3,5-dimethyl-1H-pyrazol-4-yl]methyl}benzoic acid synthesized in Example 92 and 4-(aminomethyl)pyridine as starting materials and in the same manner as in Example 83, the title compound was obtained.

¹H-NMR (CDCl₃) δ:2.18 (3H, s), 2.33 (3H, s), 3.85 (2H, s), 4.67 (2H, d), 6.50 (1H, brs), 7.19-7.30 (4H, m), 7.51 (1H, dd), 7.69-7.80 (4H, m), 8.54-8.60 (2H, m).

### Example 336

### 4-{4-[(6-bromopyridin-3-yl)methyl]-3,5-dimethyl-1H-pyrazol-1-yl}-2-chlorobenzonitrile

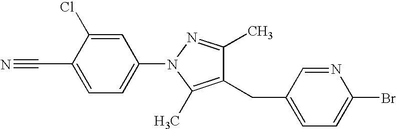

2-Bromo-5-[(3,5-dimethyl-1H-pyrazol-4-yl)methyl]pyridine (0.500 g) synthesized in Reference Example 38 was dissolved in DMF (5.0 mL), 60% sodium hydride (0.0902 g) was added under ice-cooling, and the mixture was stirred at 0° C. for 30 min. 2-Chloro-4-fluorobenzonitrile (0.585 g) was added to the reaction mixture, and the mixture was stirred at 0° C. for 1 hr. A saturated aqueous ammonium chloride solution was added to quench the reaction, and the mixture was extracted twice with ethyl acetate. The organic layers were combined, washed with saturated brine, dried over anhydrous magnesium sulfate and concentrated. The obtained residue was purified by column chromatography (hexane-ethyl acetate) and recrystallized from ethyl acetate-hexane to give the title compound (0.602 g) as a white solid.

¹H-NMR (CDCl₃) δ:2.19 (3H, s), 2.33 (3H, s), 3.72-3.79 (2H, m), 7.22-7.30 (1H, m), 7.34-7.43 (1H, m), 7.50 (1H, dd), 7.69-7.79 (2H, m), 8.19-8.26 (1H, m.).

### Example 337

### 2-chloro-4-{3,5-dimethyl-4-[4-(5-methyl-1,3,4-oxadiazol-2-yl)phenoxy]-1H-pyrazol-1-yl}benzonitrile

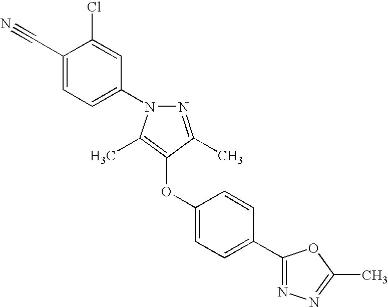

Using 4-{[1-(3-chloro-4-cyanophenyl)-3,5-dimethyl-1H-pyrazol-4-yl]oxy}benzoic acid synthesized in Example 306 and acetohydrazide as starting materials and in the same manner as in Example 303, N′-acetyl-4-{[1-(3-chloro-4-cyanophenyl)-3,5-dimethyl-1H-pyrazol-4-yl]oxy}benzohydrazide was obtained. A solution of the compound (67.1 mg) obtained above in phosphorus oxychloride (5 ml) was heated under reflux for 1 hr. The reaction mixture was concentrated and mixed with saturated aqueous sodium hydrogen carbonate, and the mixture was extracted with ethyl acetate. The ethyl acetate layer was washed with saturated aqueous sodium hydrogen carbonate and saturated brine, dried over anhydrous sodium sulfate and concentrated. The residue was purified by column chromatography (hexane-ethyl acetate) and recrystallized from ethyl acetate-hexane to give the title compound (23.2 mg) as white crystals.

¹H-NMR (CDCl₃) δ:2.16 (3H, s), 2.34 (3H, s), 2.61 (3H, s), 7.00-7.07 (2H, m), 7.57 (1H, dd), 7.77 (1H, d), 7.81 (1H, d), 7.95-8.03 (2H, m).

### Example 338

### 4,4′-{oxybis[methanediyl(3,5-dimethyl-1H-pyrazole-4,1-diyl)]}bis(2-chlorobenzonitrile)

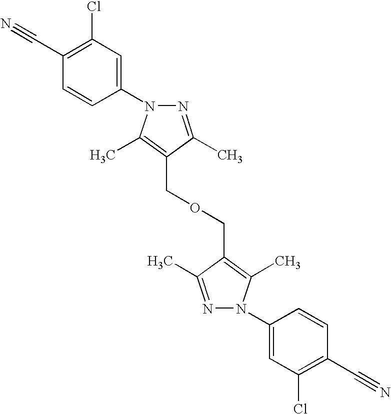

To a solution of 2-chloro-4-[4-(hydroxymethyl)-3,5-dimethyl-1H-pyrazol-1-yl]benzonitrile (100 mg) synthesized in Reference Example 21 in THF (1 ml) were added methanesulfonyl chloride (71.0 μL) and triethylamine (161 μL), and the mixture was stirred at room temperature for 2 days. The reaction mixture was mixed with methanol, and the mixture was stirred for 30 min and concentrated. The residue was diluted with ethyl acetate, washed with saturated aqueous ammonium chloride solution and saturated brine, dried over anhydrous sodium sulfate and concentrated. The residue was purified by column chromatography (hexane-ethyl acetate) and recrystallized from ethyl acetate-hexane to give the title compound (19.7 mg) as white crystals.

¹H-NMR (CDCl₃) δ:2.32 (6H, s), 2.41 (6H, s), 4.42 (4H, s), 7.48 (2H, dd), 7.70 (2H, d), 7.74 (2H, d).

### Example 339

### 4-{[1-(3-chloro-4-cyanophenyl)-3,5-dimethyl-1H-pyrazol-4-yl]oxy}-N-(2,2,2-trifluoroethyl)benzamide

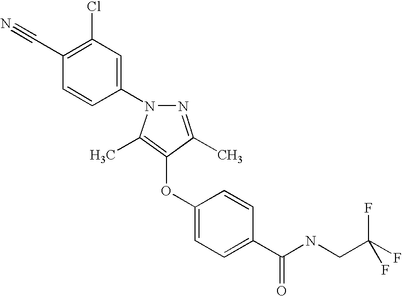

To a solution of 4-{[1-(3-chloro-4-cyanophenyl)-3,5-dimethyl-1H-pyrazol-4-yl]oxy}benzoic acid (100 mg) synthesized in Example 306 and 4-(4,6-dimethoxy-1,3,5-triazin-2-yl)-4-methylmorpholin-4-ium chloride (151 mg) in THF-2-propanol (1:1, 2 ml) were added 2,2,2-trifluoroethylamine hydrochloride (73.7 mg) and triethylamine (75.8 μL), and the mixture was stirred at room temperature for 16 hr. The reaction mixture was concentrated and mixed with 1 mol/l hydrochloric acid, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from ethyl acetate-hexane to give the title compound (62.9 mg) as white crystals.

¹H-NMR (CDCl₃) δ:2.14 (3H, s), 2.32 (3H, s), 4.06-4.20 (2H, m), 6.26-6.37 (1H, m), 6.96-7.02 (2H, m), 7.57 (1H, dd), 7.75-7.82 (4H, m).

### Example 340

### 4-{[1-(3-chloro-4-cyanophenyl)-3,5-dimethyl-1H-pyrazol-4-yl]oxy}-N-(1-methylethyl)benzamide

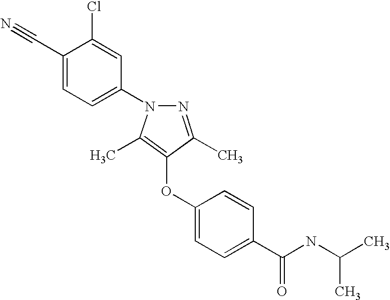

Using 4-{[1-(3-chloro-4-cyanophenyl)-3,5-dimethyl-1H-pyrazol-4-yl]oxy}benzoic acid synthesized in Example 306 and isopropylamine as starting materials and in the same manner as in Example 316, the title compound was obtained.

¹H-NMR (CDCl₃) δ:1.25 (3H, s), 1.28 (3H, s), 2.13 (3H, s), 2.31 (3H, s), 4.22-4.35 (1H, m), 5.77-5.85 (1H, m), 6.92-6.98 (2H, m), 7.56 (1H, dd), 7.70-7.78 (3H, m), 7.80 (1H, d).

### Example 341

### 4-{[1-(3-chloro-4-cyanophenyl)-3,5-dimethyl-1H-pyrazol-4-yl]oxy}-N-cyclopropylbenzamide

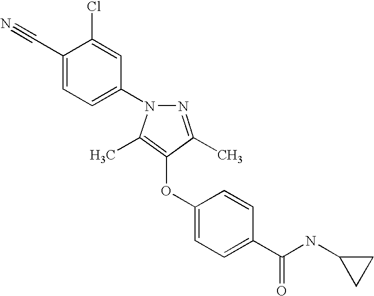

Using 4-{[1-(3-chloro-4-cyanophenyl)-3,5-dimethyl-1H-pyrazol-4-yl]oxy}benzoic acid synthesized in Example 306 and cyclopropanamine as starting materials and in the same manner as in Example 316, the title compound was obtained.

¹H-NMR (DMSO-d₆) δ:0.51-0.58 (2H, m), 0.64-0.72 (2H, m), 2.05 (3H, s), 2.29 (3H, s), 2.76-2.87 (1H, m), 6.97-7.04 (2H, m), 7.78-7.85 (3H, m), 8.01 (1H, d), 8.12 (1H, d), 8.34 (1H, d).

### Example 342

### 4-{[1-(3-chloro-4-cyanophenyl)-3,5-dimethyl-1H-pyrazol-4-yl]oxy}-N-(2-hydroxy-2-methylpropyl)benzamide

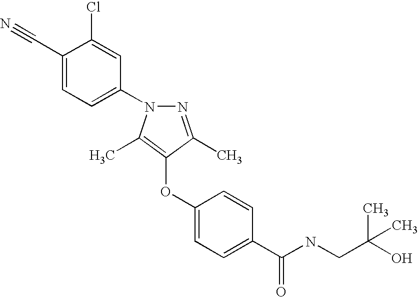

Using 4-{[1-(3-chloro-4-cyanophenyl)-3,5-dimethyl-1H-pyrazol-4-yl]oxy}benzoic acid synthesized in Example 306 and 1-amino-2-methyl-2-propanol as starting materials and in the same manner as in Example 303, the title compound was obtained.

¹H-NMR (CDCl₃) δ:1.30 (6H, s), 2.14 (3H, s), 2.19 (1H, s), 2.32 (3H, s), 3.48 (2H, d), 6.47-6.54 (1H, m), 6.94-7.00 (2H, m), 7.56 (1H, dd), 7.74-7.82 (4H, m).

### Example 343

### 4-{[1-(3-chloro-4-cyanophenyl)-3,5-dimethyl-1H-pyrazol-4-yl]oxy}-N-[(1-hydroxycyclopropyl)methyl]benzamide

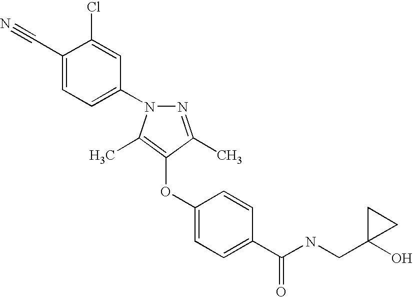

Using 4-{[1-(3-chloro-4-cyanophenyl)-3,5-dimethyl-1H-pyrazol-4-yl]oxy}benzoic acid synthesized in Example 306 and 1-(aminomethyl)cyclopropanol as starting materials and in the same manner as in Example 303, the title compound was obtained.

¹H-NMR (CDCl₃) δ:0.63-0.70 (2H, m), 0.84-0.91 (2H, m), 2.14 (3H, s), 2.32 (3H, s), 3.19 (1H, s), 3.59 (2H, d), 6.51-6.59 (1H, m), 6.94-7.01 (2H, m), 7.57 (1H, dd), 7.74-7.83 (4H, m).

### Example 344

### 4-(4-{[(6-bromopyridin-3-yl)oxy]methyl}-3,5-dimethyl-1H-pyrazol-1-yl)-2-chlorobenzonitrile

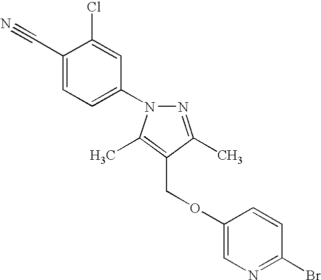

To a solution of 2-chloro-4-[4-(hydroxymethyl)-3,5-dimethyl-1H-pyrazol-1-yl]benzonitrile (100 mg) synthesized in Reference Example 21 in THF (2 ml) were added methanesulfonyl chloride (89.0 μL) and diisopropylethylamine (329 μL), and the mixture was stirred at room temperature for 30 hr. A solution of 2-bromo-5-hydroxypyridine (398 mg), which was treated with sodium hydride (91.7 mg) in advance, in THF (4 ml) was added to the above-mentioned solution, and the mixture was stirred for 5 days. The reaction mixture was ice-cooled and mixed with water, and the mixture was extracted with ethyl acetate. The ethyl acetate layer was washed with saturated aqueous sodium hydrogen carbonate and saturated brine, dried over anhydrous sodium sulfate and concentrated. The residue was purified by column chromatography (hexane-ethyl acetate) and recrystallized from ethyl acetate-hexane to give the title compound (31.9 mg) as yellow crystals.

¹H-NMR (CDCl₃) δ:2.33 (3H, s), 2.43 (3H, s), 4.92 (2H, s), 7.18 (1H, dd), 7.42 (1H, d), 7.51 (1H, dd), 7.73 (1H, d), 7.77 (1H, d), 8.14 (1H, d).

### Example 345

### 2-chloro-4-[4-(4-{[(3R)-3-hydroxypyrrolidin-1-yl]carbonyl}benzyl)-3,5-dimethyl-1H-pyrazol-1-yl]benzonitrile

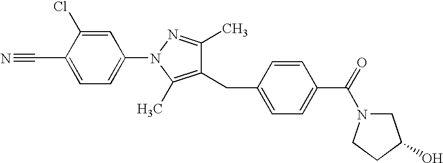

Using 4-{[1-(3-chloro-4-cyanophenyl)-3,5-dimethyl-1H-pyrazol-4-yl]methyl}benzoic acid synthesized in Example 92 and (3R)-3-hydroxypyrrolidine as starting materials and in the same manner as in Example 295, the title compound was obtained.

¹H-NMR (CDCl₃) δ:1.62-1.81 (1H, m), 1.94-2.13 (2H, m), 2.19 (3H, s), 2.33 (3H, s), 3.36-3.89 (6H, m), 4.41-4.65 (1H, m), 7.16 (2H, d), 7.41-7.57 (3H, m), 7.69-7.79 (2H, m).

### Example 346

### 2-chloro-4-(3,5-dimethyl-4-{[2-oxo-5-(trifluoromethyl)pyridin-1(2H)-yl]methyl}-1H-pyrazol-1-yl)benzonitrile

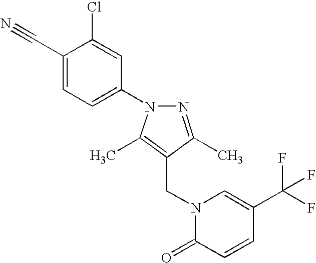

The title compound was obtained using 2-chloro-4-[4-(hydroxymethyl)-3,5-dimethyl-1H-pyrazol-1-yl]benzonitrile synthesized in Reference Example 21 and 5-(trifluoromethyl)pyridin-2-ol as starting materials, in the same manner as in Example 344, and separating highly-polar compound by column chromatography (hexane-ethyl acetate).

¹H-NMR (CDCl₃) δ:2.29 (3H, s), 2.46 (3H, s), 4.99 (2H, s), 6.66 (1H, d), 7.42-7.55 (2H, m), 7.60 (1H, brs), 7.73 (1H, d), 7.77 (1H, d).

### Example 347

### 2-chloro-4-(3,5-dimethyl-4-{[(6-methylpyridin-3-yl)oxy]methyl}-1H-pyrazol-1-yl)benzonitrile

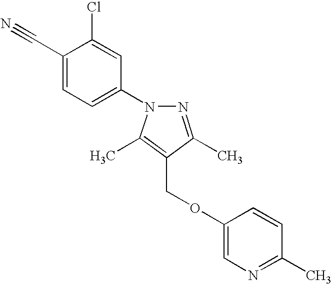

Using 2-chloro-4-[4-(hydroxymethyl)-3,5-dimethyl-1H-pyrazol-1-yl]benzonitrile synthesized in Reference Example 21 and 2-methyl-5-hydroxypyridine as starting materials and in the same manner as in Example 344, the title compound was obtained.

¹H-NMR (CDCl₃) δ:2.34 (3H, s), 2.43 (3H, s), 2.52 (3H, s), 4.91 (2H, s), 7.07-7.14 (1H, m), 7.17-7.23 (1H, m), 7.51 (1H, dd), 7.73 (1H, d), 7.76 (1H, d), 8.27 (1H, brs).

### Example 348

### 5-{[1-(3-chloro-4-cyanophenyl)-3,5-dimethyl-1H-pyrazol-4-yl]methyl}pyridine-2-carboxamide

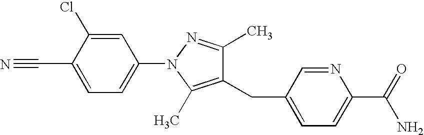

### Step 1

4-{4-[(6-Bromopyridin-3-yl)methyl]-3,5-dimethyl-1H-pyrazol-1-yl}-2-chlorobenzonitrile (0.100 g) synthesized in Example 336 was dissolved in methanol (3.0 mL), and triethylamine (0.0416 mL) and [1,1′-bis(diphenylphosphino)ferrocene]palladium(II)dichloride dichloromethane complex (0.0203 g) were added under an argon atmosphere. The gaseous phase was substituted with carbon monoxide (1 atm), and the mixture was heated at 60° C. for 6 hr. The reaction mixture was allowed to cool and concentrated under reduced pressure. The residue was neutralized with saturated aqueous sodium hydrogen carbonate solution, and the mixture was extracted twice with ethyl acetate. The organic layers were combined, washed with saturated brine, dried over anhydrous magnesium sulfate and concentrated. The obtained residue was purified by column chromatography (hexane-ethyl acetate) to give methyl 5-{[1-(3-chloro-4-cyanophenyl)-3,5-dimethyl-1H-pyrazol-4-yl]methyl}pyridine-2-carboxylate (0.058 g) as a white solid.

### Step 2

Methyl 5-{[1-(3-chloro-4-cyanophenyl)-3,5-dimethyl-1H-pyrazol-4-yl]methyl}pyridine-2-carboxylate (0.058 g) obtained in step 1 was dissolved in THF (1.0 ml)-methanol (1.0 mL), 1 mol/l aqueous sodium hydroxide solution (1.0 mL) was added, and the mixture was stirred at room temperature for 1 hr. The reaction mixture was neutralized with 1 mol/l hydrochloric acid, and the mixture was extracted twice with ethyl acetate. The organic layers were combined, washed with saturated aqueous sodium hydrogen carbonate solution and saturated brine, dried over anhydrous magnesium sulfate and concentrated to give 5-{[1-(3-chloro-4-cyanophenyl)-3,5-dimethyl-1H-pyrazol-4-yl]methyl}pyridine-2-carboxylic acid (0.0442 g) as a white solid.

### Step 3

5-{[1-(3-Chloro-4-cyanophenyl)-3,5-dimethyl-1H-pyrazol-4-yl]methyl}pyridine-2-carboxylic acid (0.0442 g) obtained in step 2 was dissolved in DMF (1.0 mL), N-[3-(dimethylamino)propyl]-N′-ethylcarbodiimide hydrochloride (0.0347 g) and N-hydroxybenzotriazole ammonium salt (0.0449 g) were added, and the mixture was stirred at room temperature for 19 hr. The reaction mixture was neutralized with 10% aqueous sodium hydrogensulfate solution, and the mixture was extracted twice with ethyl acetate. The organic layers were combined, washed with saturated aqueous sodium hydrogen carbonate solution and saturated brine, dried over anhydrous magnesium sulfate and concentrated. The obtained residue was purified by column chromatography (hexane-ethyl acetate), and the obtained solid was recrystallized from hexane-ethyl acetate to give the title compound (0.0291 g) as a white solid.

¹H-NMR (300 MHz, DMSO-d₆) δ:2.12 (3H, s), 2.41 (3H, s), 3.91 (2H, s), 7.59 (1H, brs), 7.68-7.80 (2H, m), 7.93-7.98 (2H, m), 8.03 (1H, brs), 8.09 (1H, d), 8.51 (1H, d).

### Example 349

### 2-chloro-4-[3,5-dimethyl-4-({[5-(trifluoromethyl)pyridin-2-yl]oxy}methyl)-1H-pyrazol-1-yl]benzonitrile

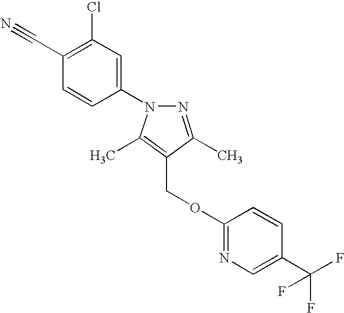

The reaction of Example 346 was performed, and the resulting less polar compound was separated by column chromatography (hexane-ethyl acetate) to give the title compound.

¹H-NMR (CDCl₃) δ:2.37 (3H, s), 2.49 (3H, s), 5.31 (2H, s), 6.83 (1H, d), 7.51 (1H, dd), 7.70-7.82 (3H, m), 8.44-8.50 (1H, m).

### Example 350

### 5-(4-{[1-(3-chloro-4-cyanophenyl)-3,5-dimethyl-1H-pyrazol-4-yl]methyl}phenyl)-1,3,4-oxadiazole-2-carboxamide

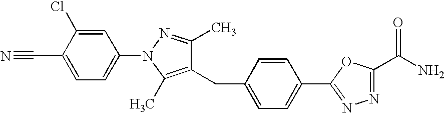

2-{2-[(4-{[1-(3-Chloro-4-cyanophenyl)-3,5-dimethyl-1H-pyrazol-4-yl]methyl}phenyl)carbonyl]hydrazino}-2-oxoacetamide (0.868 g) obtained in Example 325, step 1, was suspended in xylene (10.0 mL), p-toluenesulfonyl chloride (0.551 g) and triethylamine (0.403 mL) were added, and the mixture was heated under reflux for 17 hr. DMF (5.0 mL) and triethylamine (0.403 mL) were added to the reaction mixture, and the mixture was further heated under reflux for 7 hr. The reaction mixture was allowed to cool and concentrated under reduced pressure. A saturated aqueous sodium hydrogen carbonate solution was added to the residue, and the mixture was extracted twice with ethyl acetate. The organic layers were combined and washed with saturated brine. During the partitioning operation, the precipitated solid was collected by filtration, the organic layer of the filtrate was dried over anhydrous magnesium sulfate and concentrated under reduced pressure. The residue was suspended in a mixed solvent of toluene-acetone, the insoluble solid was collected by filtration to give the title compound (0.119 g). The filtrate was purified by column chromatography (hexane-ethyl acetate) to give the title compound (0.078 g). In addition, the solid precipitated during the partitioning operation was collected by filtration, suspended in THF, filtered and insoluble material was removed. The filtrate was concentrated, and the residue was purified by column chromatography (hexane-ethyl acetate) to give the title compound (0.270 g) as a white solid.

melting point 223-226° C.

¹H-NMR (DMSO-d₆) δ:2.21 (3H, s), 2.42 (3H, m), 3.91 (2H, s), 7.44 (2H, d), 7.76 (1H, dd), 7.96 (1H, dd), 8.00 (2H, d), 8.09 (1H, d), 8.25 (1H, brs), 8.66 (1H, brs).

### Example 351

### N-tert-butyl-4-{[1-(3-chloro-4-cyanophenyl)-3,5-dimethyl-1H-pyrazol-4-yl]methyl}benzamide

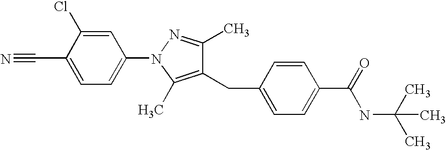

A mixture of 4-{[1-(3-chloro-4-cyanophenyl)-3,5-dimethyl-1H-pyrazol-4-yl]methyl}benzoic acid (2.00 g) synthesized in Example 92, N-[3-(dimethylamino)propyl]-N′-ethylcarbodiimide hydrochloride (1.57 g), 1-hydroxybenzotriazole (1.11 g), tert-butylamine (0.862 mL) and DMF (20 mL) was stirred at room temperature for 13 hr. 10% Aqueous sodium hydrogensulfate solution was added to the reaction mixture, and the mixture was extracted twice with ethyl acetate. The organic layers were combined, washed with 1 mol/l hydrochloric acid, saturated aqueous sodium hydrogen carbonate, water and saturated brine, dried over anhydrous magnesium sulfate and concentrated. The obtained residue was purified by column chromatography (hexane-ethyl acetate), and the obtained oil was recrystallized from ethyl acetate-hexane to give the title compound (1.81 g) as a white solid.

melting point 135-136° C.

¹H-NMR (CDCl₃) δ:1.46 (9H, s), 2.17 (3H, s), 2.32 (3H, s), 3.82 (2H, s), 5.89 (1H, brs), 7.17 (2H, d), 7.51 (1H, dd), 7.62-7.67 (2H, m), 7.74 (2H, dd).

### Example 352

### 4-({1-[4-cyano-3-(trifluoromethyl)phenyl]-3,5-dimethyl-1H-pyrazol-4-yl}methyl)-N-(2-hydroxy-2-methylpropyl)benzamide

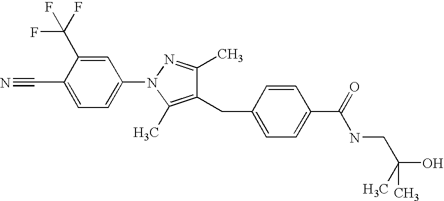

Using 4-({1-[4-cyano-3-(trifluoromethyl)phenyl]-3,5-dimethyl-1H-pyrazol-4-yl}methyl)benzoic acid synthesized in Example 81 and 1-amino-2-methyl-2-propanol as starting materials and in the same manner as in Example 295, the title compound was obtained.

¹H-NMR (CDCl₃) δ:1.29 (6H, s), 2.13 (1H, s), 2.19 (3H, s), 2.35 (3H, s), 3.48 (2H, d), 3.85 (2H, s), 6.52 (1H, t), 7.21 (2H, d), 7.71-7.76 (2H, m), 7.79 (1H, dd), 7.88-7.94 (1H, m), 8.02 (1H, d).

### Example 353

### 2-chloro-4-(3,5-dimethyl-4-{4-[(4-oxopiperidin-1-yl)carbonyl]benzyl}-1H-pyrazol-1-yl)benzonitrile

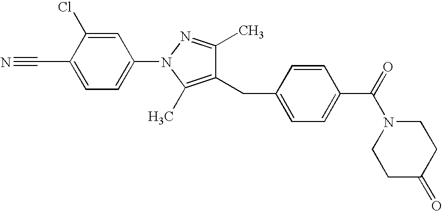

Using 4-{[1-(3-chloro-4-cyanophenyl)-3,5-dimethyl-1H-pyrazol-4-yl]methyl}benzoic acid synthesized in Example 92, piperidin-4-one hydrochloride monohydrate and triethylamine as starting materials and in the same manner as in Example 295, the title compound was obtained.

¹H-NMR (CDCl₃) δ:2.20 (3H, s), 2.34 (3H, s), 2.38-2.67 (4H, m), 3.67-4.13 (4H, m), 3.84 (2H, s), 7.20 (2H, d), 7.37-7.44 (2H, m), 7.52 (1H, dd), 7.72-7.76 (2H, m).

### Example 354

### 2-chloro-4-(4-{4-[(4-hydroxy-4-phenylpiperidin-1-yl)carbonyl]benzyl}-3,5-dimethyl-1H-pyrazol-1-yl)benzonitrile

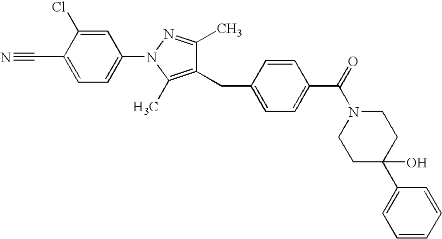

Using 4-{[1-(3-chloro-4-cyanophenyl)-3,5-dimethyl-1H-pyrazol-4-yl]methyl}benzoic acid synthesized in Example 92 and 4-hydroxy-4-phenylpiperidine as starting materials and in the same manner as in Example 295, the title compound was obtained.

¹H-NMR (CDCl₃) δ:1.66-2.02 (4H, m), 2.20 (3H, s), 2.33 (3H, s), 3.30-3.76 (4H, m), 3.82 (2H, s), 4.56-4.79 (1H, m), 7.17 (2H, d), 7.28-7.33 (1H, m), 7.35-7.43 (4H, m), 7.44-7.56 (3H, m), 7.69-7.78 (2H, m).

### Example 355

### 2-chloro-4-(4-{4-[(4-hydroxypiperidin-1-yl)carbonyl]benzyl}-3,5-dimethyl-1H-pyrazol-1-yl)benzonitrile

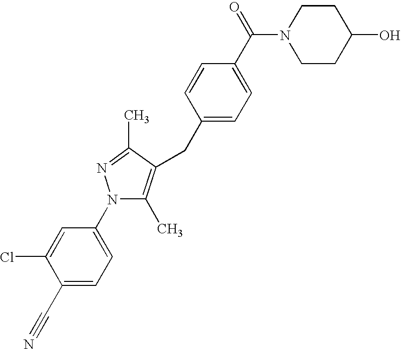

A mixture of 4-{[1-(3-chloro-4-cyanophenyl)-3,5-dimethyl-1H-pyrazol-4-yl]methyl}benzoic acid (2.50 g) synthesized in Example 92, 4-hydroxypiperidine (0.830 g), 4-(4,6-dimethoxy-1,3,5-triazin-2-yl)-4-methylmorpholin-4-ium chloride (2.27 g), THF (25 mL) and 2-propanol (25 mL) was stirred at room temperature for 19 hr. 4-Hydroxypiperidine (0.100 g) and 4-(4,6-dimethoxy-1,3,5-triazin-2-yl)-4-methylmorpholin-4-ium chloride (0.200 g) were added to the reaction mixture, and the mixture was stirred at room temperature for 24 hr. The reaction mixture was concentrated under reduced pressure. 10% Aqueous sodium hydrogensulfate solution was added to the residue, and the mixture was extracted twice with ethyl acetate. The organic layers were combined, washed with saturated aqueous sodium hydrogen carbonate and saturated brine, dried over anhydrous magnesium sulfate and concentrated. The residue was purified by column chromatography (methanol-ethyl acetate) and recrystallized from ethyl acetate-hexane to give the title compound (2.75 g) as a white solid.

melting point 153-154° C.

¹H-NMR (CDCl₃) δ:1.51 (1H, d), 1.89 (2H, brs), 2.20 (3H, s), 2.33 (3H, s), 3.05-4.37 (6H, m), 3.81 (2H, s), 3.98 (1H, td), 7.13-7.22 (2H, m), 7.30-7.36 (2H, m), 7.51 (1H, dd), 7.71-7.76 (2H, m).

### Example 356

### 4-{[1-(3-chloro-4-cyanophenyl)-3,5-dimethyl-1H-pyrazol-4-yl]methyl}-N-(2-hydroxy-2-methylpropyl)benzamide

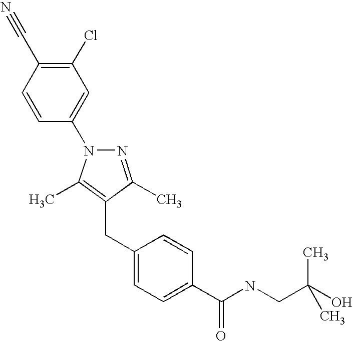

A mixture of 4-{[1-(3-chloro-4-cyanophenyl)-3,5-dimethyl-1H-pyrazol-4-yl]methyl}benzoic acid (1.00 g) synthesized in Example 92, 1-amino-2-methylpropan-2-ol (487 mg), 4-(4,6-dimethoxy-1,3,5-triazin-2-yl)-4-methylmorpholin-4-ium chloride (1.13 g), THF (10 mL) and 2-propanol (10 mL) was stirred at room temperature for 24 hr. The reaction mixture was concentrated under reduced pressure and mixed with 1 mol/l hydrochloric acid, and the mixture was extracted with ethyl acetate. The organic layer was washed with saturated aqueous ammonium chloride solution and saturated brine, dried over anhydrous sodium sulfate and concentrated. The obtained residue was purified by column chromatography (hexane-ethyl acetate) and recrystallized from ethyl acetate-hexane to give the title compound (1.03 g) as a white solid.

melting point 171-172° C.

¹H-NMR (CDCl₃) δ:1.29 (6H, s), 2.16 (1H, s), 2.18 (3H, s), 2.32 (3H, s), 3.48 (2H, d), 3.84 (2H, s), 6.53 (1H, brs), 7.21 (2H, d), 7.51 (1H, dd), 7.70-7.76 (4H, m).

### Example 357

### 2-chloro-4-{3,5-dimethyl-4-[4-(morpholin-4-ylcarbonyl)phenoxy]-1H-pyrazol-1-yl}benzonitrile

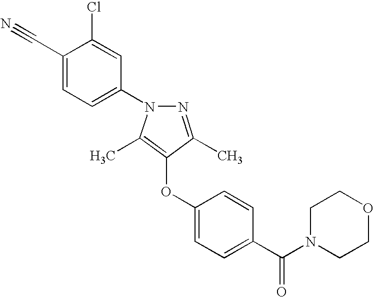

A solution of 4-{[1-(3-chloro-4-cyanophenyl)-3,5-dimethyl-1H-pyrazol-4-yl]oxy}benzoic acid (1.80 g) synthesized in Example 306, 1-hydroxybenzotriazole (0.992 g), morpholine (0.642 mL) and N-(3-dimethylaminopropyl)-N′-ethylcarbodiimide hydrochloride (1.41 g) in DMF (20 mL) was stirred at room temperature for 17 hr. The reaction mixture was ice-cooled and mixed with 1 mol/l hydrochloric acid, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate). The obtained white solid (1.98 g) and a separately synthesized product (1.33 g) were mixed and recrystallized from ethyl acetate-hexane to give the title compound (2.93 g) as white crystals.

melting point 156-157° C.

¹H-NMR (CDCl₃) δ:2.15 (3H, s), 2.31 (3H, s), 3.70 (8H, brs), 6.92-6.98 (2H, m), 7.37-7.43 (2H, m), 7.56 (1H, dd), 7.76 (1H, d), 7.79 (1H, d).

### Example 358

### 2-chloro-4-(4-{4-[(4-hydroxypiperidin-1-yl)carbonyl]phenoxy}-3,5-dimethyl-1H-pyrazol-1-yl)benzonitrile

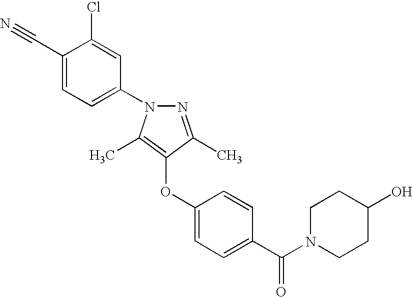

Using 4-{[1-(3-chloro-4-cyanophenyl)-3,5-dimethyl-1H-pyrazol-4-yl]oxy}benzoic acid synthesized in Example 306 and 4-hydroxypiperidine as starting materials and in the same manner as in Example 303, the title compound was obtained.

¹H-NMR (CDCl₃) δ:1.47-1.62 (3H, m), 1.94 (2H, brs), 2.15 (3H, s), 2.31 (3H, s), 3.31 (2H, brs), 3.55-4.41 (3H, m), 6.94 (2H, d), 7.39 (2H, d), 7.56 (1H, d), 7.72-7.82 (2H, m).

### Example 359

### 2-chloro-4-(3,5-dimethyl-4-{4-[(4-methylpiperazin-1-yl)carbonyl]phenoxy}-1H-pyrazol-1-yl)benzonitrile

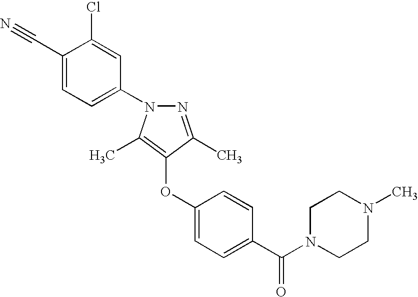

Using 4-{[1-(3-chloro-4-cyanophenyl)-3,5-dimethyl-1H-pyrazol-4-yl]oxy}benzoic acid synthesized in Example 306 and 1-methylpiperazine as starting materials, a reaction was performed by the method shown in Example 303 using diisopropyl ether as a recrystallization solvent to give the title compound.

¹H-NMR (CDCl₃) δ:2.15 (3H, s), 2.31 (3H, s), 2.33 (3H, s), 2.42 (4H, brs), 3.64 (4H, brs), 6.90-6.97 (2H, m), 7.36-7.43 (2H, m), 7.56 (1H, dd), 7.76 (1H, d), 7.79 (1H, d).

### Example 360

### 4-{[1-(3-chloro-4-cyanophenyl)-3,5-dimethyl-1H-pyrazol-4-yl]oxy}-N-(2-hydroxy-1,1-dimethylethyl)benzamide

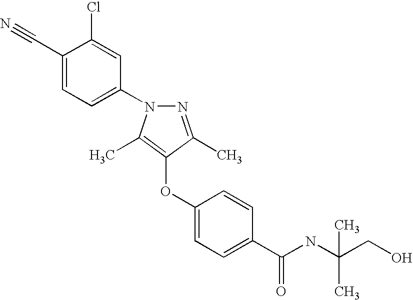

Using 4-{[1-(3-chloro-4-cyanophenyl)-3,5-dimethyl-1H-pyrazol-4-yl]oxy}benzoic acid synthesized in Example 306 and 2-amino-2-methyl-1-propanol as starting materials and in the same manner as in Example 303, the title compound was obtained.

¹H-NMR (CDCl₃) δ:1.42 (6H, s), 2.13 (3H, s), 2.31 (3H, s), 3.70 (2H, d), 4.55 (1H, t), 6.08 (1H, s), 6.92-6.99 (2H, m), 7.56 (1H, dd), 7.68-7.74 (2H, m), 7.76 (1H, d), 7.80 (1H, d).

### Example 361

### N-tert-butyl-5-{[1-(3-chloro-4-cyanophenyl)-3,5-dimethyl-1H-pyrazol-4-yl]methyl}pyridine-2-carboxamide

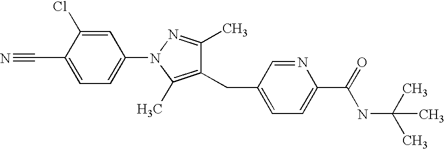

A mixture of 5-{[1-(3-chloro-4-cyanophenyl)-3,5-dimethyl-1H-pyrazol-4-yl]methyl}pyridine-2-carboxylic acid (0.057 g) obtained in Example 348, step 1, N-[3-(dimethylamino)propyl]-N′-ethylcarbodiimide hydrochloride (0.0447 g), N-hydroxybenzotriazole (0.0315 g), tert-butylamine (0.0245 mL) and DMF (1.0 mL) was stirred at room temperature for 3 days. The reaction mixture was neutralized with 10% aqueous sodium hydrogensulfate solution, and the mixture was extracted twice with ethyl acetate. The organic layers were combined, washed with saturated aqueous sodium hydrogen carbonate solution and saturated brine, dried over anhydrous magnesium sulfate and concentrated. The obtained residue was purified by column chromatography (hexane-ethyl acetate) to give the title compound (0.053 g) as a colorless oil.

¹H-NMR (CDCl₃) δ:1.48 (9H, s), 2.18 (3H, s), 2.33 (3H, s), 3.84 (2H, s), 7.50 (1H, dd), 7.56 (1H, dd), 7.72 (2H, d), 7.92 (1H, brs), 8.10 (1H, d), 8.33 (1H, d).

### Example 362

### 5-{[1-(3-chloro-4-cyanophenyl)-3,5-dimethyl-1H-pyrazol-4-yl]methyl}-N-(2-hydroxy-2-methylpropyl)pyridine-2-carboxamide

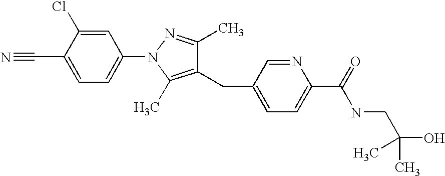

A mixture of 5-{[1-(3-chloro-4-cyanophenyl)-3,5-dimethyl-1H-pyrazol-4-yl]methyl}pyridine-2-carboxylic acid (0.72 g) obtained in Example 348, step 2, 1-amino-2-methyl-2-propanol (0.021 g), 4-(4,6-dimethoxy-1,3,5-triazin-2-yl)-4-methylmorpholin-4-ium chloride (0.081 g), THF (1.0 mL) and 2-propanol (1.0 mL) was stirred at room temperature for 3 days. The reaction mixture was neutralized with 10% aqueous sodium hydrogensulfate solution, and the mixture was extracted twice with ethyl acetate. The organic layers were combined, washed with saturated aqueous sodium hydrogen carbonate solution and saturated brine, dried over anhydrous magnesium sulfate and concentrated. The obtained residue was purified by column chromatography (hexane-ethyl acetate) to give the title compound (0.079 g) as a colorless oil.

¹H-NMR (CDCl₃) δ:1.29 (6H, s), 2.19 (3H, s), 2.34 (3H, s), 2.48 (1H, s), 3.48 (2H, d), 3.86 (2H, s), 7.51 (1H, dd), 7.58 (1H, dd), 7.71-7.78 (2H, m), 8.13 (1H, d), 8.29-8.46 (2H, m).

### Example 363

### 2-chloro-4-(4-{4-[(3-hydroxyazetidin-1-yl)carbonyl]benzyl}-3,5-dimethyl-1H-pyrazol-1-yl)benzonitrile

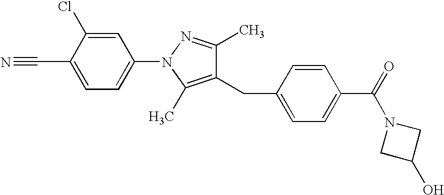

A mixture of 4-{[1-(3-chloro-4-cyanophenyl)-3,5-dimethyl-1H-pyrazol-4-yl]methyl}benzoic acid (0.100 g) synthesized in Example 92, 3-hydroxyazetidine hydrochloride (0.0599 g), triethylamine (0.0762 mL), 4-(4,6-dimethoxy-1,3,5-triazin-2-yl)-4-methylmorpholin-4-ium chloride (0.151 g), THF (1.0 mL) and 2-propanol (1.0 mL) was stirred at room temperature for 2 days. The reaction mixture was concentrated under reduced pressure. 10% Aqueous sodium hydrogensulfate solution was added to the residue, and the mixture was extracted twice with ethyl acetate. The organic layers were combined, washed with saturated aqueous sodium hydrogen carbonate and saturated brine, dried over anhydrous magnesium sulfate and concentrated. The residue was purified by column chromatography (methanol-ethyl acetate), the obtained oil was left standing at room temperature to allow solidification to give the title compound (0.102 g) as a white solid.

melting point 115-117° C.

¹H-NMR (CDCl₃) δ:2.17 (3H, s), 2.32 (3H, s), 3.59-3.75 (1H, m), 3.81 (2H, s), 3.93-4.24 (2H, m), 4.35-4.50 (2H, m), 4.61-4.72 (1H, m), 7.17 (2H, d), 7.47-7.57 (3H, m), 7.73 (2H, dd).

### Example 364

### 4-{[1-(3-chloro-4-cyanophenyl)-3,5-dimethyl-1H-pyrazol-4-yl]methyl}-N-[2-hydroxy-1-(hydroxymethyl)ethyl]benzamide

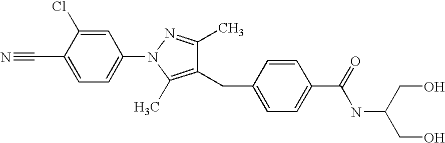

Using 4-{[1-(3-chloro-4-cyanophenyl)-3,5-dimethyl-1H-pyrazol-4-yl]methyl}benzoic acid synthesized in Example 92 and 2-aminopropane-1,3-diol as starting materials and in the same manner as in Example 295, the title compound was obtained.

¹H-NMR (CDCl₃) δ:2.18 (3H, s), 2.32 (3H, s), 2.50 (2H, dd), 3.84 (2H, s), 3.86-4.06 (4H, m), 4.10-4.22 (1H, m), 6.84-6.93 (1H, m), 7.21 (2H, d), 7.51 (1H, dd), 7.74 (4H, td).

### Example 365

### 2-chloro-4-(4-{4-[(4-hydroxy-4-methylpiperidin-1-yl)carbonyl]benzyl}-3,5-dimethyl-1H-pyrazol-1-yl)benzonitrile

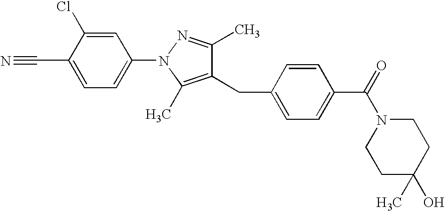

2-Chloro-4-(3,5-dimethyl-4-{4-[(4-oxopiperidin-1-yl)carbonyl]benzyl}-1H-pyrazol-1-yl)benzonitrile (0.084 g) obtained in Example 353 was dissolved in THF (1.0 mL), a solution (1.4 mol/l, 0.268 mL) of methylmagnesium bromide in toluene-THF was added under ice-cooling, and the mixture was stirred at 0° C. for 1 hr. The reaction mixture was neutralized with saturated aqueous ammonium chloride solution, and the mixture was extracted twice with ethyl acetate. The organic layers were combined, washed with saturated brine, dried over anhydrous magnesium sulfate and concentrated. The obtained residue was purified by column chromatography (ethyl acetate-methanol) and recrystallized from diisopropyl ether to give the title compound (0.035 g) as a white solid.

¹H-NMR (CDCl₃) δ:1.30 (3H, s), 1.46-1.79 (4H, m), 2.20 (3H, s), 2.33 (3H, s), 3.27-4.44 (4H, m), 3.82 (2H, s), 7.15 (2H, d), 7.33 (2H, d), 7.51 (1H, dd), 7.70-7.79 (2H, m).

### Example 366

### 4-{[1-(3-chloro-4-cyanophenyl)-3,5-dimethyl-1H-pyrazol-4-yl]oxy}-N-(pyridin-2-ylmethyl)benzamide

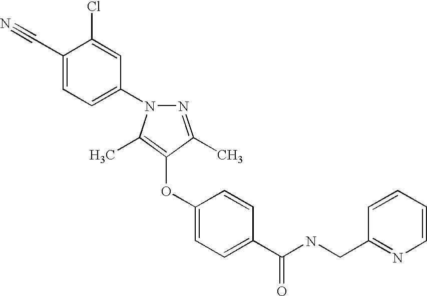

Using 4-{[1-(3-chloro-4-cyanophenyl)-3,5-dimethyl-1H-pyrazol-4-yl]oxy}benzoic acid synthesized in Example 306 and 1-pyridin-2-ylmethaneamine as starting materials and in the same manner as in Example 316, the title compound was obtained.

¹H-NMR (CDCl₃) δ:2.13 (3H, s), 2.31 (3H, s), 4.75 (2H, d), 6.94-7.00 (2H, m), 7.19-7.24 (1H, m), 7.30-7.35 (1H, m), 7.48-7.53 (1H, m), 7.56 (1H, dd), 7.65-7.72 (1H, m), 7.75 (1H, d), 7.79 (1H, d), 7.81-7.88 (2H, m), 8.52-8.58 (1H, m).

### Example 367

### 2-chloro-4-(4-{4-[(1,1-dioxidethiomorpholin-4-yl)carbonyl]phenoxy}-3,5-dimethyl-1H-pyrazol-1-yl)benzonitrile

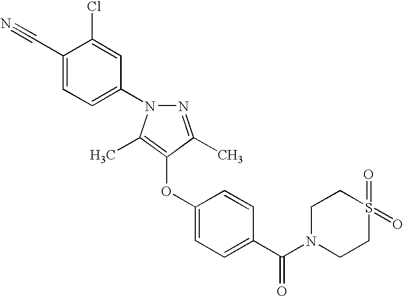

A solution of 4-{[1-(3-chloro-4-cyanophenyl)-3,5-dimethyl-1H-pyrazol-4-yl]oxy}benzoic acid (750 mg) synthesized in Example 306, 1-hydroxybenzotriazole (413 mg), thiomorpholine 1,1-dioxide (414 mg) and N-(3-dimethylaminopropyl)-N′-ethylcarbodiimide hydrochloride (587 mg) in DMF (15 mL) was stirred at room temperature for 16 hr. The reaction mixture was ice-cooled and mixed with 1 mol/l hydrochloric acid, and the insoluble solid was collected by filtration. The filtrate was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate). The solid obtained by filtration and the solid obtained by column purification were mixed, and recrystallized from ethyl acetate-hexane to give the title compound (695 mg) as white crystals.

melting point 218-219 ° C.

¹H-NMR (CDCl₃) δ:2.15 (3H, s), 2.32 (3H, s), 3.08 (4H, brs), 4.12 (4H, brs), 6.99 (2H, d), 7.43 (2H, d), 7.56 (1H, d), 7.73-7.82 (2H, m).

### Example 368

### 2-chloro-4-[4-(4-{[(2R,6S)-2,6-dimethylmorpholin-4-yl]carbonyl}phenoxy)-3,5-dimethyl-1H-pyrazol-1-yl]benzonitrile

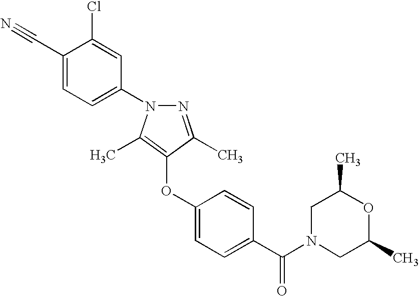

Using 4-{[1-(3-chloro-4-cyanophenyl)-3,5-dimethyl-1H-pyrazol-4-yl]oxy}benzoic acid synthesized in Example 306 and 2,6-cis-dimethylmorpholine as starting materials and in the same manner as in Example 316, the title compound was obtained.

¹H-NMR (CDCl₃) δ:1.19 (6H, brs), 2.15 (3H, s), 2.32 (3H, s), 2.53-2.84 (2H, m), 3.44-3.79 (3H, m), 4.51 (1H, brs), 6.91-6.98 (2H, m), 7.36-7.42 (2H, m), 7.56 (1H, dd), 7.76 (1H, d), 7.79 (1H, d).

### Example 369

### 4-{[1-(3-chloro-4-cyanophenyl)-3,5-dimethyl-1H-pyrazol-4-yl]methyl}-N-[2-(methylsulfanyl)ethyl]benzamide

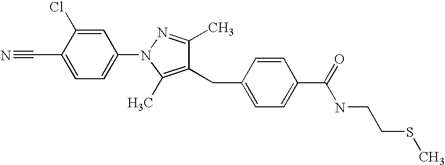

Using 4-{[1-(3-chloro-4-cyanophenyl)-3,5-dimethyl-1H-pyrazol-4-yl]methyl}benzoic acid synthesized in Example 92 and 2-(methylthio)ethylamine as starting materials and in the same manner as in Example 83, the title compound was obtained.

¹H-NMR (CDCl₃) δ:2.14 (3H, s), 2.18 (3H, s), 2.33 (3H, s), 2.76 (2H, t), 3.67 (2H, q), 3.84 (2H, s), 6.55 (1H, brs), 7.20 (2H, d), 7.51 (1H, dd), 7.74 (4H, dd).

### Example 370

### 4-{[1-(3-chloro-4-cyanophenyl)-3,5-dimethyl-1H-pyrazol-4-yl]oxy}-N-(4-methylphenyl)benzamide

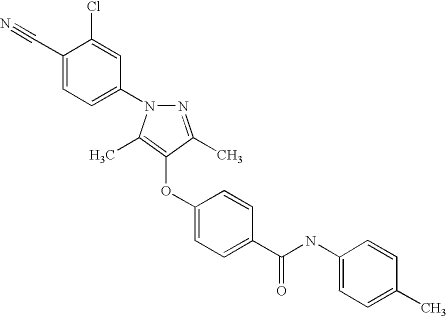

A solution of 4-{[1-(3-chloro-4-cyanophenyl)-3,5-dimethyl-1H-pyrazol-4-yl]oxy}benzoic acid (100 mg) synthesized in Example 306 and O-(7-azabenzotriazol-1-yl)-N,N,N′,N′-tetramethyluronium hexafluorophosphate (155 mg), p-toluidine (43.7 mg) and N-(3-dimethylaminopropyl)-N′-ethylcarbodiimide hydrochloride (78.2 mg) in DMF (1 ml) was stirred at room temperature for 16 hr. The reaction mixture was ice-cooled and mixed with 1 mol/l hydrochloric acid, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from ethyl acetate-hexane to give the title compound (47.4 mg) as white crystals.

¹H-NMR (DMSO-d₆) δ:2.08 (3H, s), 2.27 (3H, s), 2.32 (3H, s), 7.04-7.19 (4H, m), 7.63 (2H, d), 7.82 (1H, dd), 7.91-7.99 (2H, m), 8.02 (1H, d), 8.14 (1H, d), 10.08 (1H, s).

### Example 371

### 5-(4-{[1-(3-chloro-4-cyanophenyl)-3,5-dimethyl-1H-pyrazol-4-yl]oxy}phenyl)-1,3,4-oxadiazole-2-carboxamide

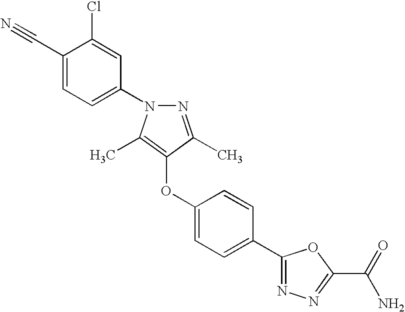

Using 4-{[1-(3-chloro-4-cyanophenyl)-3,5-dimethyl-1H-pyrazol-4-yl]oxy}benzoic acid synthesized in Example 306 and oxamic acid hydrazide as starting materials, the reaction in Example 303 was performed using DMF as a solvent to give 2-{2-[(4-{[1-(3-chloro-4-cyanophenyl)-3,5-dimethyl-1H-pyrazol-4-yl]oxy}phenyl)carbonyl]hydrazino}-2-oxoacetamide. To a solution of the compound (765 mg) above in toluene-DMF (7:3, 10 ml) were added p-toluenesulfonyl chloride (967 mg) and triethylamine (707 μL), and the mixture was heated at 120° C. for 40 min using microwave. The reaction mixture was concentrated and mixed with saturated aqueous sodium hydrogen carbonate, and the mixture was extracted with ethyl acetate. The ethyl acetate layer was washed with saturated aqueous sodium hydrogen carbonate and saturated brine, dried over anhydrous sodium sulfate and concentrated. The residue was purified by column chromatography (hexane-ethyl acetate) and recrystallized from acetone-hexane to give the title compound (81.1 mg) as white crystals.

¹H-NMR (DMSO-d₆) δ:2.09 (3H, s), 2.32 (3H, s), 7.19-7.26 (2H, 25 m), 7.82 (1H, dd), 8.03 (1H, d), 8.04-8.10 (2H, m), 8.14 (1H, d), 8.24 (1H, s), 8.66 (1H, s).

### Example 372

### methyl 4-({1-[4-cyano-3-(trifluoromethyl)phenyl]-3,5-dimethyl-1H-pyrazol-4-yl}oxy)benzoate

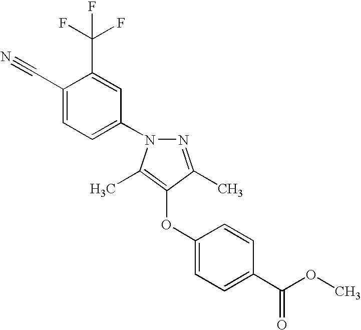

Using methyl 4-[(3,5-dimethyl-1H-pyrazol-4-yl)oxy]benzoate synthesized in Reference Example 16 and 2-trifluoromethyl-4-fluorobenzonitrile as starting materials and in the same manner as in Example 305, the title compound was obtained.

¹H-NMR (CDCl₃) δ:2.15 (3H, s), 2.35 (3H, s), 3.91 (3H, s), 6.93-7.00 (2H, m), 7.82-7.88 (1H, m), 7.91-7.97 (1H, m), 7.98-8.05 (2H, m), 8.08 (1H, d).

### Example 373

### 4-({1-[4-cyano-3-(trifluoromethyl)phenyl]-3,5-dimethyl-1H-pyrazol-4-yl}oxy)benzoic acid

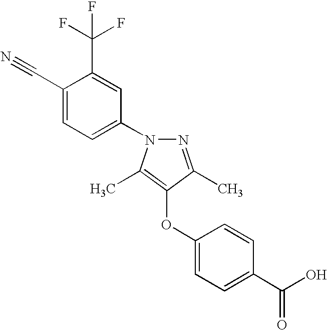

Using methyl 4-({1-[4-cyano-3-(trifluoromethyl)phenyl]-3,5-dimethyl-1H-pyrazol-4-yl}oxy)benzoate synthesized in Example 372 as a starting material and in the same manner as in Example 306, the title compound was obtained.

¹H-NMR (CDCl₃) δ:2.16 (3H, s), 2.35 (3H, s), 6.96-7.03 (2H, m), 7.80-7.90 (1H, m), 7.92-7.98 (1H, m), 8.05-8.13 (3H, m).

### Example 374

### (±)-4-{[1-(3-chloro-4-cyanophenyl)-3,5-dimethyl-1H-pyrazol-4-yl]methyl}-N-[2-(methylsulfinyl)ethyl]benzamide

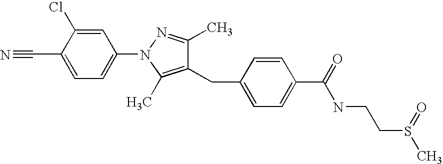

4-{[1-(3-Chloro-4-cyanophenyl)-3,5-dimethyl-1H-pyrazol-4-yl]methyl}-N-[2-(methylsulfanyl)ethyl]benzamide (0.100 g) obtained in Example 369 was dissolved in acetonitrile (10.0 mL)-ethyl acetate (5.0 mL), 3-chloroperbenzoic acid (0.0524 g) was added under ice-cooling, and the mixture was stirred at 0° C. for 2 hr. The reaction mixture was removed from the ice-bath, and further reacted at room temperature for 20 hr. The reaction mixture was neutralized with saturated aqueous sodium hydrogen carbonate solution and concentrated under reduced pressure, and the mixture was extracted twice with ethyl acetate. The organic layers were combined, washed with saturated brine, dried over anhydrous magnesium sulfate and concentrated. The residue was purified by column chromatography (ethyl acetate-methanol) to give the title compound (0.0218 g) as a white solid.

¹H-NMR (CDCl₃) δ:2.17 (3H, s), 2.32 (3H, s), 2.68 (3H, s), 2.87 (1H, ddd), 3.15 (1H, ddd), 3.82 (2H, s), 3.93-4.18 (2H, m), 7.19 (2H, d), 7.29-7.36 (1H, m), 7.51 (1H, dd), 7.68-7.78 (4H, m).

### Example 375

### 4-{[1-(3-chloro-4-cyanophenyl)-3,5-dimethyl-1H-pyrazol-4-yl]methyl}-N-[2-(methylsulfonyl)ethyl]benzamide

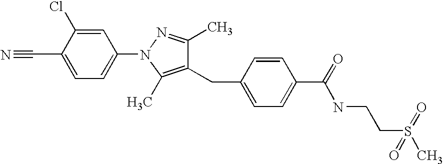

4-{[1-(3-Chloro-4-cyanophenyl)-3,5-dimethyl-1H-pyrazol-4-yl]methyl}-N-[2-(methylsulfanyl)ethyl]benzamide (0.190 g) obtained in Example 369 was dissolved in acetonitrile (4.0 mL)-ethyl acetate (2.0 mL), and 3-chloroperbenzoic acid (0.250 g) was added under ice-cooling. The reaction mixture was removed from the ice-bath, and stirred at room temperature for 1 day. The reaction mixture was neutralized with saturated aqueous sodium hydrogen carbonate solution, and the mixture was concentrated under reduced pressure and extracted twice with ethyl acetate. The organic layers were combined, washed with saturated brine, dried over anhydrous magnesium sulfate and concentrated. The residue was washed with diisopropyl ether to give the title compound (0.138 g) as a white solid.

¹H-NMR (CDCl₃) δ:2.18 (3H, s), 2.32 (3H, s), 3.00 (3H, s), 3.35 (2H, d), 3.83 (2H, s), 3.96-4.06 (2H, m), 6.96 (1H, brs), 7.21 (2H, d), 7.51 (1H, dd), 7.67-7.79 (4H, m).

### Example 376

### 4-{[1-(3-chloro-4-cyanophenyl)-3,5-dimethyl-1H-pyrazol-4-yl]oxy}-N-[6-(trifluoromethyl)pyridin-3-yl]benzamide

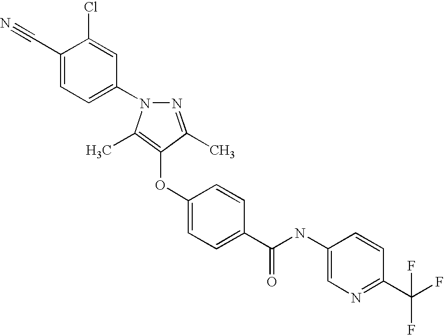

The title compound was obtained using 4-{[1-(3-chloro-4-cyanophenyl)-3,5-dimethyl-1H-pyrazol-4-yl]oxy}benzoic acid synthesized in Example 306 and 6-(trifluoromethyl)pyridine-3-amine as starting materials, in the same manner as in Example 370, and adding 4-dimethylaminopyridine (0.1 equivalent amount).

¹H-NMR (CDCl₃) δ:2.15 (3H, s), 2.34 (3H, s), 7.02-7.09 (2H, m), 7.57 (1H, dd), 7.73 (1H, d), 7.77 (1H, d), 7.81 (1H, d), 7.86-7.92 (3H, m), 8.55 (1H, dd), 8.72 (1H, d).

### Example 377

### 4-{3,5-dimethyl-4-[4-(5-methyl-1,3,4-oxadiazol-2-yl)phenoxy]-1H-pyrazol-1-yl}-2-(trifluoromethyl)benzonitrile

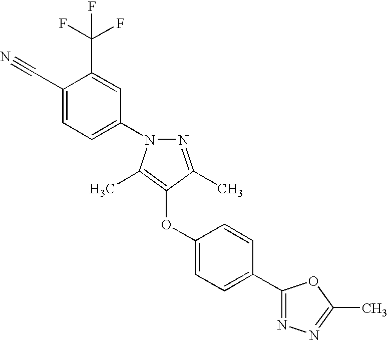

Using 4-({1-[4-cyano-3-(trifluoromethyl)phenyl]-3,5-dimethyl-1H-pyrazol-4-yl}oxy)benzoic acid synthesized in Example 373 and acetohydrazide as starting materials and in the same manner as in Example 303, N′-acetyl-4-({1-[4-cyano-3-(trifluoromethyl)phenyl]-3,5-dimethyl-1H-pyrazol-4-yl}oxy)benzohydrazide was obtained. To a solution of the above-mentioned compound (230 mg) in xylene (5 ml) was added p-toluenesulfonic acid monohydrate (95.6 mg), and the mixture was stirred at 160° C. for 2 hr using a Dean-stark. The reaction mixture was concentrated and mixed with saturated aqueous sodium hydrogen carbonate, and the mixture was extracted with ethyl acetate. The ethyl acetate layer was washed with saturated aqueous sodium hydrogen carbonate and saturated brine, dried over anhydrous sodium sulfate and concentrated. The residue was purified by column chromatography (hexane-ethyl acetate) and recrystallized from ethyl acetate-hexane to give the title compound (12.8 mg) as white crystals.

¹H-NMR (CDCl₃) δ:2.17 (3H, s), 2.36 (3H, s), 2.61 (3H, s), 7.01-7.07 (2H, m), 7.83-7.88 (1H, m), 7.92-7.96 (1H, m), 7.96-8.02 (2H, m), 8.09 (1H, d).

### Example 378

### 5-(4-{[1-(3-chloro-4-cyanophenyl)-3,5-dimethyl-1H-pyrazol-4-yl]methyl}phenyl)-N-methyl-1,3,4-oxadiazole-2-carboxamide

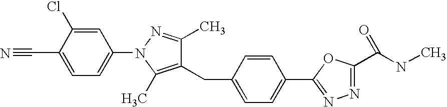

To a suspension (2.0 mL) of methyl 5-(4-{[1-(3-chloro-4-cyanophenyl)-3,5-dimethyl-1H-pyrazol-4-yl]methyl}phenyl)-1,3,4-oxadiazole-2-carboxylate (0.0434 g) synthesized in Example 381 in THF was added methylamine-THF solution (2.0 mol/L, 0.48 mL) under ice-cooling, and the mixture was stirred at room temperature for 5 hr. The reaction mixture was concentrated under reduced pressure to give a crude product (0.0457 g). The crude product was combined with a crude product (0.0277 g) separately obtained by a similar operation, and suspended in a mixed solvent of toluene-acetone. The insoluble solid was collected by filtration to give the title compound (0.0366 g) as a white solid.

¹H-NMR (DMSO-d₆) δ:2.12 (3H, s), 2.42 (3H, s), 2.82 (3H, d), 3.91 (2H, s), 7.44 (2H, d), 7.76 (1H, dd), 7.96 (1H, d), 8.00 (2H, d), 8.09 (1H, d), 9.21-9.31 (1H, m).

### Example 379

### 4-{[1-(3-chloro-4-cyanophenyl)-3,5-dimethyl-1H-pyrazol-4-yl]oxy}-N-(2-hydroxy-1,1,2-trimethylpropyl)benzamide

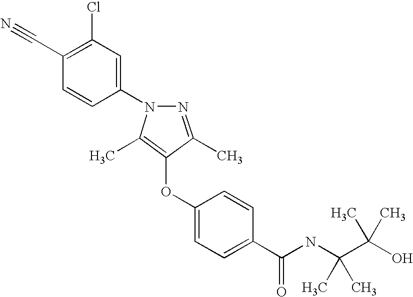

Methyl N-[(4-{[1-(3-chloro-4-cyanophenyl)-3,5-dimethyl-1H-pyrazol-4-yl]oxy}phenyl)carbonyl]-2-amino-2-methylpropanoate (80.0 mg) obtained in Example 40 was dissolved in THF (2.5 mL), a solution (1.0 mol/L, 0.857 mL) of methylmagnesium bromide in THF was added under ice-cooling, and the mixture was stirred at 0° C. for 2 hr and at room temperature for 19 hr. Methylmagnesium bromide-THF solution (1.0 mol/L, 0.857 mL) was added to the reaction mixture, and the mixture was stirred at room temperature for 6 hr and neutralized with 1 mol/l hydrochloric acid. The mixture was extracted twice with ethyl acetate. The organic layers were combined, washed with saturated brine, dried over anhydrous magnesium sulfate and concentrated. The obtained residue was purified by column chromatography (hexane-ethyl acetate) and recrystallized from ethyl acetate-hexane to give the title compound (55.2 mg) as a white solid.

¹H-NMR (CDCl₃) δ:1.24 (6H, s), 1.45 (6H, s), 2.13 (3H, s), 2.31 (3H, s), 5.24 (1H, s), 6.21 (1H, brs), 6.93-6.99 (2H, m), 7.56 (1H, dd), 7.69-7.78 (3H, m), 7.80 (1H, d).

### Example 380

### methyl N-[(4-{[1-(3-chloro-4-cyanophenyl)-3,5-dimethyl-1H-pyrazol-4-yl]oxy}phenyl)carbonyl]aminoacetate

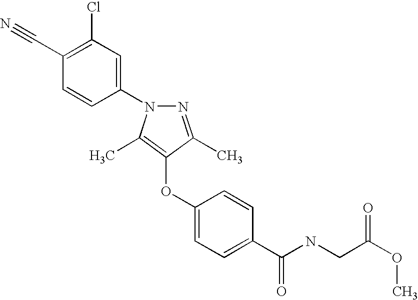

A solution of 4-{[1-(3-chloro-4-cyanophenyl)-3,5-dimethyl-1H-pyrazol-4-yl]oxy}benzoic acid (500 mg) synthesized in Example 306, 1-hydroxybenzotriazole (276 mg), glycine methyl ester hydrochloride (256 mg), N-(3-dimethylaminopropyl)-N′-ethylcarbodiimide hydrochloride (391 mg) and triethylamine (0.284 mL) in DMF (6 mL) was stirred at room temperature for 19 hr. The reaction mixture was ice-cooled and mixed with 1 mol/l hydrochloric acid, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from methyl ethyl ketone-hexane to give the title compound (189 mg) as white crystals.

¹H-NMR (CDCl₃) δ:2.14 (3H, s), 2.32 (3H, s), 3.81 (3H, s), 4.25 (2H, d), 6.54 (1H, brs), 6.97 (2H, d), 7.58 (1H, s), 7.73-7.85 (4H, m).

### Example 381

### methyl 5-(4-{[1-(3-chloro-4-cyanophenyl)-3,5-dimethyl-1H-pyrazol-4-yl]methyl}phenyl)-1,3,4-oxadiazole-2-carboxylate

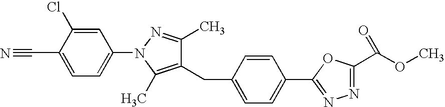

A mixture of 4-{[1-(3-chloro-4-cyanophenyl)-3,5-dimethyl-1H-pyrazol-4-yl]methyl}benzoic acid (1.22 g) obtained in Example 92, tert-butyl carbazate (0.722 g), 4-(4,6-dimethoxy-1,3,5-triazin-2-yl)-4-methylmorpholin-4-ium chloride (0.907 g) and DMF (20 mL) was stirred at room temperature for 24 hr. The reaction mixture was concentrated under reduced pressure, the residue was mixed with water, and the mixture was extracted twice with ethyl acetate. The organic layers were combined, washed with saturated brine, dried over anhydrous magnesium sulfate and concentrated. The residue was purified by column chromatography (hexane-ethyl acetate) to give a white solid (1.06 g). The obtained solid (1.06 g) was suspended in hydrogen chloride-ethyl acetate solution (4 mol/L, 10 mL), and the mixture was stirred at room temperature for 2 hr. The reaction mixture was concentrated under reduced pressure to give a white solid (1.06 g). The obtained solid (1.06 g) was suspended in THF (20.0 mL), triethylamine (1.54 mL) and methyl chloroglyoxylate (0.224 mL) were added under ice-cooling, and the mixture was stirred at room temperature for 6 hr. Methyl chloroglyoxylate (0.081 mL) was added to the mixture, and the mixture was stirred at room temperature for 4 hr. p-Toluenesulfonyl chloride (0.843 g) was added to the mixture, and the mixture was stirred at room temperature for 13 hr and at 50° C. for 6 hr. Triethylamine (1.54 mL) and p-toluenesulfonyl chloride (0.400 g) were added to the mixture, and the mixture was stirred at 50° C. for 4 hr and at room temperature for 4 days. The reaction mixture was concentrated under reduced pressure, saturated aqueous sodium hydrogen carbonate was added to the residue, and the mixture was extracted twice with ethyl acetate. The organic layers were combined, washed with saturated brine, dried over anhydrous magnesium sulfate and concentrated. The residue was suspended in a mixed solvent of toluene-acetone, and the insoluble solid was collected by filtration to give the title compound (0.382 g) as a white solid. The filtrate was purified by column chromatography (hexane-ethyl acetate) and recrystallized from ethyl acetate-hexane to give the title compound (0.056 g) as a white solid.

¹H-NMR (CDCl₃) δ:2.19 (3H, s), 2.35 (3H, s), 3.89 (2H, s), 4.09 (3H, s), 7.31 (2H, d), 7.53 (1H, dd), 7.71-7.78 (2H, m), 8.05-8.13 (2H, m).

### Example 382

### 4-{4-[4-(5-acetyl-1,3,4-oxadiazol-2-yl)benzyl]-3,5-dimethyl-1H-pyrazol-1-yl}-2-chlorobenzonitrile

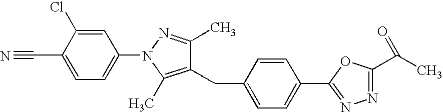

Methyl 5-(4-{[1-(3-chloro-4-cyanophenyl)-3,5-dimethyl-1H-pyrazol-4-yl]methyl}phenyl)-1,3,4-oxadiazole-2-carboxylate (0.100 g) synthesized in Example 381 was suspended in THF (2.0 mL), methylmagnesium bromide (toluene-THF solution, 1.4M, 0.638 mL) was added under ice-cooling, and the mixture was stirred at 0° C. for 60 min. Methylmagnesium bromide (toluene-THF solution, 1.4M, 0.319 mL) and THF (2.0 mL) were added to the reaction mixture, and the mixture was stirred for 30 min under ice-cooling and at room temperature for 30 min. A saturated aqueous ammonium chloride solution was added to the reaction mixture, and the mixture was extracted twice with ethyl acetate. The organic layers were combined, washed with saturated brine, dried over anhydrous magnesium sulfate and concentrated under reduced pressure. The residue was purified by column chromatography (hexane-ethyl acetate) to give the title compound (0.0253 g, white solid).

¹H-NMR (DMSO-d₆) δ:2.12 (3H, s), 2.42 (3H, s), 2.69 (3H, s), 3.92 (2H, s), 7.45 (2H, d), 7.76 (1H, dd), 7.96 (1H, d), 8.02 (2H, d), 8.09 (1H, d).

### Example 383

### 2-chloro-4-(4-{4-[5-(1-hydroxy-1-methylethyl)-1,3,4-oxadiazol-2-yl]benzyl}-3,5-dimethyl-1H-pyrazol-1-yl)benzonitrile

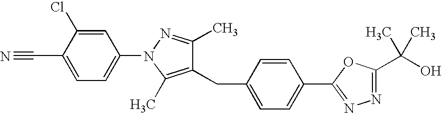

A fraction containing the title compound was concentrated under reduced pressure in the purification step by column chromatography in Example 382 to give a colorless oil. The oil was mixed with diisopropyl ether to allow solidification, and the solid was collected by filtration to give the title compound (0.0255 g) as a white solid.

¹H-NMR (DMSO-d₆) δ:1.60 (6H, s), 2.11 (3H, s), 2.41 (3H, s), 3.90 (2H, s), 5.91 (1H, s), 7.41 (2H, d), 7.75 (1H, dd), 7.89-7.98 (3H, m), 8.09 (1H, d).

### Example 384

### 5-(4-{[1-(3-chloro-4-cyanophenyl)-3,5-dimethyl-1H-pyrazol-4-yl]methyl}phenyl)-N,N-dimethyl-1,3,4-oxadiazole-2-carboxamide

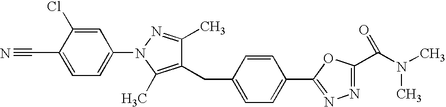

Methyl 5-(4-{[1-(3-chloro-4-cyanophenyl)-3,5-dimethyl-1H-pyrazol-4-yl]methyl}phenyl)-1,3,4-oxadiazole-2-carboxylate (0.100 g) synthesized in Example 381 was suspended in THF (2.0 mL), dimethylamine-THF solution (2.0 mol/L, 1.12 mL) was added, and the mixture was stirred at room temperature for 14 hr. The mixture was diluted with THF (2.0 mL), and stirred at 50° C. for 1 hr and heated under reflux for 4 hr. DMF (1.0 mL) and dimethylamine-THF solution (2.0 mol/L, 1.12 mL) were added to the mixture, and the mixture was heated under reflux for 4 hr. Dimethylamine-THF solution (2.0 mol/L, 1.12 mL) was added to the mixture, and the mixture was heated under reflux for 14 hr. The reaction mixture was cooled to room temperature and concentrated under reduced pressure. The residue was dissolved again in DMF (1.0 mL), and placed in a microwave reaction vessel. Dimethylamine-THF solution (2.0 mol/L, 1.12 mL) was added, and the mixture was stirred at 100° C. for 60 min and at 120° C. for 30 min under microwave irradiation. The reaction mixture was cooled to room temperature, concentrated under reduced pressure and mixed with saturated aqueous sodium hydrogen carbonate. The mixture was extracted twice with ethyl acetate. The organic layers were combined, washed with saturated brine, dried over anhydrous magnesium sulfate and concentrated under reduced pressure. The residue was purified by column chromatography (hexane-ethyl acetate) to give the title compound (0.0125 g) as a white solid.

¹H-NMR (DMSO-d₆) δ:2.12 (3H, s), 2.42 (3H, s), 3.07 (3H, s), 3.35 (3H, s), 3.92 (2H, s), 7.44 (2H, d), 7.76 (1H, dd), 7.93-8.03 (3H, m), 8.09 (1H, d).

### Example 385

### N-(2-amino-2-oxoethyl)-4-{[1-(3-chloro-4-cyanophenyl)-3,5-dimethyl-1H-pyrazol-4-yl]oxy}benzamide

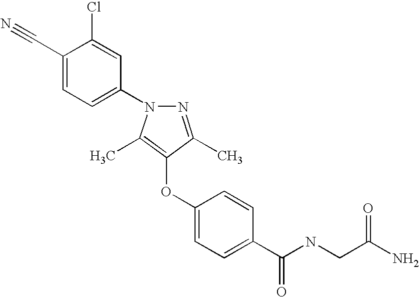

A solution of N-[(4-{[1-(3-chloro-4-cyanophenyl)-3,5-dimethyl-1H-pyrazol-4-yl]oxy}phenyl)carbonyl]glycine (100 mg) synthesized in Example 464, 1-hydroxybenzotriazole ammonium salt (87.7 mg) and N-(3-dimethylaminopropyl)-N′-ethylcarbodiimide hydrochloride (67.7 mg) in DMF (2 mL) was stirred at room temperature for 17 hr. The reaction mixture was ice-cooled and mixed with 1 mol/l hydrochloric acid, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (ethyl acetate-methanol) to give a white solid. The white solid was washed with toluene and recrystallized from ethyl acetate-hexane to give the title compound (44.5 mg) as white crystals.

¹H-NMR (DMSO-d₆) δ:2.06 (3H, s), 2.30 (3H, s), 3.79 (2H, d), 6.97-7.09 (3H, m), 7.35 (1H, brs), 7.81 (1H, dd), 7.85-7.93 (2H, m), 8.02 (1H, d), 8.13 (1H, d), 8.59 (1H, t).

### Example 386

### 4-{(1-(3-chloro-4-cyanophenyl)-3,5-dimethyl-1H-pyrazol-4-yl)oxy}-N-(2-(methylamino)-2-oxoethyl)benzamide

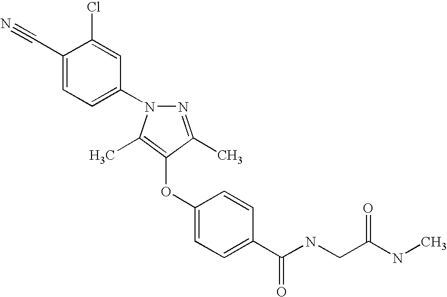

A solution of N-[(4-{(1-(3-chloro-4-cyanophenyl)-3,5-dimethyl-1H-pyrazol-4-yl)oxy}phenyl)carbonyl]glycine (100 mg) synthesized in Example 464, 1-hydroxybenzotriazole (47.7 mg), methylamine-THF solution (2.0 mol/L, 0.590 mL) and N-(3-dimethylaminopropyl)-N′-ethylcarbodiimide hydrochloride (67.7 mg) in DMF (2 mL) was stirred at room temperature for 17 hr. The reaction mixture was ice-cooled and mixed with 1 mol/l hydrochloric acid, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (ethyl acetate-methanol) and recrystallized from ethyl acetate-hexane to give the title compound (21.8 mg) as white crystals.

¹H-NMR (DMSO-d₆) δ:2.06 (3H, s), 2.30 (3H, s), 2.59 (3H, d), 3.81 (2H, d), 7.02-7.08 (2H, m), 7.78-7.85 (2H, m), 7.86-7.93 (2H, m), 8.02 (1H, d), 8.13 (1H, d), 8.67 (1H, t).

### Example 387

### 2-chloro-4-{4-[(4-cyanobenzyl)oxy]-3,5-dimethyl-1H-pyrazol-1-yl}benzonitrile

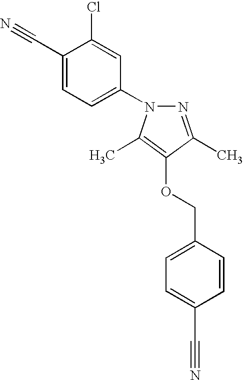

A solution of 2-chloro-4-(4-hydroxy-3,5-dimethyl-1H-pyrazol-1-yl)benzonitrile (100 mg) synthesized in Reference Example 18, 4-(bromomethyl)benzonitrile (119 mg) and potassium carbonate (83.8 mg) in DMF (4 mL) was stirred at 50° C. for 4 hr. The reaction mixture was allowed to cool and mixed with saturated aqueous ammonium chloride solution, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from ethyl acetate-hexane to give the title compound (60.6 mg) as yellow crystals.

¹H-NMR (CDCl₃) δ:2.25 (3H, s), 2.28 (3H, s), 4.95 (2H, s), 7.48 (1H, dd), 7.54 (2H, d), 7.68-7.75 (4H, m).

### Example 388

### methyl 4-({[1-(3-chloro-4-cyanophenyl)-3,5-dimethyl-1H-pyrazol-4-yl]oxy}methyl)benzoate

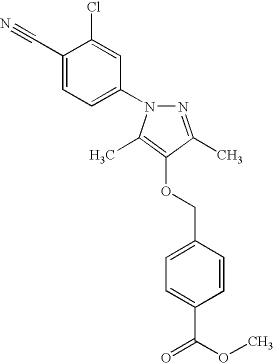

Using methyl 4-(bromomethyl) as a starting material and in the same manner as in Example 387, the title compound was obtained.

¹H-NMR (CDCl₃) δ:2.23 (3H, s), 2.24 (3H, s), 3.94 (3H, s), 4.95 (2H, s), 7.44-7.51 (3H, m), 7.68-7.74 (2H, m), 8.04-8.10 (2H, m).

### Example 389

### 2-chloro-4-(4-{hydroxy[6-(2-oxopyrrolidin-1-yl)pyridin-3-yl]methyl}-3,5-dimethyl-1H-pyrazol-1-yl)benzonitrile

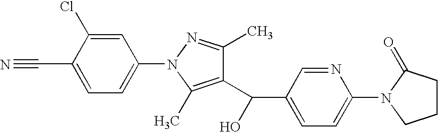

A mixture of 4-{4-[(6-bromopyridin-3-yl)(hydroxy)methyl]-3,5-dimethyl-1H-pyrazol-1-yl}-2-chlorobenzonitrile (0.0818 g) synthesized in Example 392, 2-pyrrolidone (0.0217 g), tripotassium phosphate (0.0832 g), N,N′-dimethylethylenediamine (0.00207 g), copper(I) iodide (0.00225 g) and toluene (2.0 mL) was stirred at 80° C. for 2 hr and at 110° C. for 4 hr. N,N′-Dimethylethylenediamine (0.00207 g) and copper(I) iodide (0.00225 g) were added to the reaction mixture, and the mixture was stirred at 110° C. for 13 hr. The reaction mixture was placed in a microwave reaction vessel, N,N′-dimethylethylenediamine (0.0041 g), copper(I) iodide (0.0045 g) and toluene (2.0 mL) were added, and the mixture was stirred at 130° C. for 20 min under microwave irradiation. The reaction mixture was concentrated under reduced pressure and mixed with saturated aqueous sodium hydrogen carbonate, and the mixture was extracted twice with ethyl acetate. The organic layers were combined, washed with diluted aqueous ammonia and saturated brine, dried over anhydrous magnesium sulfate and concentrated under reduced pressure. The residue was dissolved in a mixed solvent of methanol (2.0 mL)-THF (1.0 mL), sodium borohydride (0.0111 g) was added under ice-cooling, and the mixture was stirred at 0° C. for 45 min. A saturated aqueous ammonium chloride solution was added to the reaction mixture, and the mixture was extracted twice with ethyl acetate. The organic layers were combined, washed with saturated brine, dried over anhydrous magnesium sulfate and concentrated under reduced pressure. The residue was purified by column chromatography (hexane-ethyl acetate) to give the title compound (0.0217 g) as a white solid.

¹H-NMR (CDCl₃) δ:2.09-2.18 (2H, m), 2.20 (3H, s), 2.24 (1H, d), 2.36 (3H, s), 2.67 (2H, t), 4.10 (2H, t), 5.95 (1H, d), 7.47 (1H, dd), 7.66-7.79 (3H, m), 8.36 (1H, d), 8.40 (1H, d).

### Example 390

### 4-({[1-(3-chloro-4-cyanophenyl)-3,5-dimethyl-1H-pyrazol-4-yl]oxy}methyl)benzoic acid

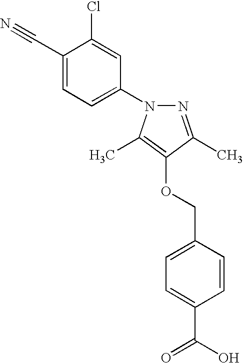

A solution of methyl 4-({[1-(3-chloro-4-cyanophenyl)-3,5-dimethyl-1H-pyrazol-4-yl]oxy}methyl)benzoate (460 mg) synthesized in Example 388 in THF-methanol (1:1, 30 mL) was heated to 40° C., 1 mol/L aqueous sodium hydroxide solution (4.60 mL) was added, and the mixture was stirred for 2 hr. The reaction mixture was allowed to cool, mixed with 1 mol/l hydrochloric acid and concentrated. The residue was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from ethyl acetate-hexane to give the title compound (128 mg) as a white solid.

¹H-NMR (DMSO-d₆) δ:2.14 (3H, s), 2.30 (3H, s), 4.97 (2H, s), 7.53 (2H, d), 7.71 (1H, d), 7.87-8.00 (3H, m), 8.07 (1H, d), 13.05 (1H, brs).

### Example 391

### 5-(4-{[1-(3-chloro-4-cyanophenyl)-3,5-dimethyl-1H-pyrazol-4-yl]methyl}phenyl)-N-(2-hydroxyethyl)-1,3,4-oxadiazole-2-carboxamide

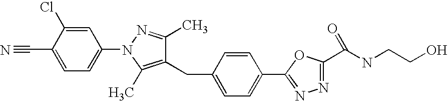

Methyl 5-(4-{[1-(3-chloro-4-cyanophenyl)-3,5-dimethyl-1H-pyrazol-4-yl]methyl}phenyl)-1,3,4-oxadiazole-2-carboxylate (0.100 g) synthesized in Example 381 was suspended in THF (2.0 mL), 2-aminoethanol (0.135 mL) was added, and the mixture was stirred at room temperature for 16 hr. The reaction mixture was concentrated under reduced pressure and mixed with 1 mol/l hydrochloric acid, and the mixture was extracted twice with ethyl acetate. The insoluble solid precipitated in the partitioning step was collected by filtration to give the title compound (0.050 g) as a white solid. The organic layers were combined, washed with saturated aqueous sodium hydrogen carbonate and saturated brine, dried over anhydrous magnesium sulfate and concentrated under reduced pressure. The residue was purified by column chromatography (methanol-ethyl acetate) to give the title compound (0.020 g) as a white solid.

¹H-NMR (DMSO-d₆) δ:2.12 (3H, s), 2.42 (3H, s), 3.33-3.40 (2H, m), 3.54 (2H, q), 3.92 (2H, s), 4.79 (1H, t), 7.44 (2H, d), 7.76 (1H, dd), 7.96 (1H, d), 8.01 (2H, d), 8.09 (1H, d), 9.21 (1H, t).

### Example 392

### 4-{4-[(6-bromopyridin-3-yl)(hydroxy)methyl]-3,5-dimethyl-1H-pyrazol-1-yl}-2-chlorobenzonitrile

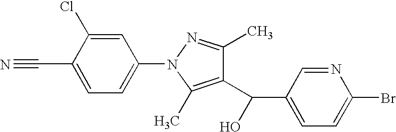

2,5-Dibromopyridine (1.55 g) was suspended in diethyl ether (60 mL), and the mixture was cooled to −78° C. in hexane-dry ice bath. n-Butyllithium-hexane solution (1.6M, 4.81 mL) was added to the mixture, and the mixture was stirred at −78° C. for 30 min. 2-Chloro-4-(4-formyl-3,5-dimethyl-1H-pyrazol-1-yl)benzonitrile (1.00 g) synthesized in Reference Example 17 and THF (60 mL) were successively added to the mixture, and the mixture was stirred at −78° C. for 30 min. The reaction mixture was warmed to room temperature over 30 min, and the mixture was further stirred at room temperature for 30 min. The reaction mixture was ice-cooled and neutralized with saturated aqueous ammonium chloride solution, and the mixture was extracted twice with ethyl acetate. The organic layers were combined, washed with saturated brine, dried over anhydrous magnesium sulfate and concentrated under reduced pressure. The residue was purified by column chromatography (hexane-ethyl acetate) to give the title compound (0.393 g) as a colorless oil.

¹H-NMR (CDCl₃) δ:2.19 (3H, s), 2.28 (1H, brs), 2.35 (3H, s), 5.93 (1H, s), 7.44-7.51 (2H, m), 7.58 (1H, dd), 7.69 (1H, d), 7.76 (1H, d), 8.38 (1H, d).

### Example 393

### 4-{4-[(6-bromopyridin-3-yl)carbonyl]-3,5-dimethyl-1H-pyrazol-1-yl}-2-chlorobenzonitrile

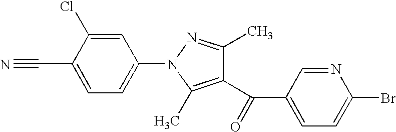

A fraction containing the title compound was concentrated under reduced pressure in the purification step by column chromatography in Example 392 to give a white solid (0.150 g). The solid was washed with diethyl ether to give the title compound (0.047 g) as a white solid.

¹H-NMR (CDCl₃) δ:2.23 (3H, s), 2.44 (3H, s), 7.53 (1H, dd), 7.67 (1H, dd), 7.75 (1H, d), 7.83 (1H, d), 7.94 (1H, dd), 8.68 (1H, dd).

### Example 394

### 2-chloro-4-{4-[4-(5-cyclopropyl-1,3,4-oxadiazol-2-yl)phenoxy]-3,5-dimethyl-1H-pyrazol-1-yl}benzonitrile

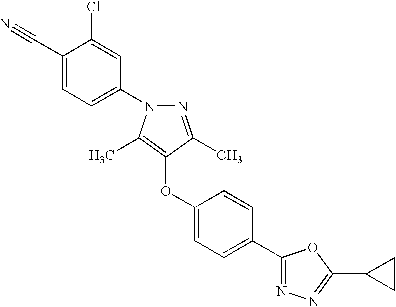

A solution of 4-{[1-(3-chloro-4-cyanophenyl)-3,5-dimethyl-1H-pyrazol-4-yl]oxy}benzohydrazide (65.6 mg) synthesized in Example 465 and cyclopropanecarboxylic acid (0.350 mL) in phosphorus oxychloride (3.5 mL) was stirred at 100° C. for 1 hr, and the mixture was concentrated. The residue was mixed with saturated aqueous sodium hydrogen carbonate solution, and the mixture was extracted with ethyl acetate. The ethyl acetate layer was washed with saturated aqueous sodium hydrogen carbonate solution and saturated brine, dried over anhydrous sodium sulfate and concentrated. The residue was purified by column chromatography (hexane-ethyl acetate) and recrystallized from ethyl acetate-hexane to give the title compound (21.5 mg) as white crystals.

¹H-NMR (CDCl₃) δ:1.15-1.22 (4H, m), 2.15 (3H, s), 2.17-2.27 (1H, m), 2.33 (3H, s), 6.99-7.05 (2H, m), 7.57 (1H, dd), 7.77 (1H, d), 7.81 (1H, d), 7.92-7.99 (2H, m).

### Example 395

### 2-chloro-4-{4-[(2,6-dioxo-1,2,3,6-tetrahydropyrimidin-4-yl)methoxy]-3,5-dimethyl-1H-pyrazol-1-yl}benzonitrile

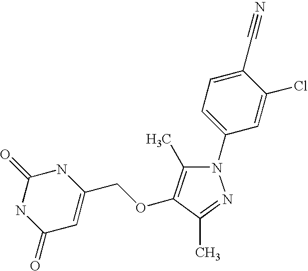

A solution (0.120M, 0.500 mL) of 2-chloro-4-(4-hydroxy-3,5-dimethyl-1H-pyrazol-1-yl)benzonitrile synthesized in Reference Example 18 in DMF, a solution (0.180M, 0.500 mL) of 6-(chloromethyl)pyrimidine-2,4(1H,3H)-dione in DMF and potassium carbonate (12.4 mg) were added to an MCS reaction vessel, and the mixture was stirred at 50° C. for 16 hr. The reaction mixture was allowed to cool to room temperature, and the mixture was extracted with ethyl acetate (3 mL) and water (2 mL). The organic layer was separated by upper layer phase sep tube (manufactured by Wako Pure Chemical Industries, Ltd.). The solvent was evaporated under reduced pressure, and the residue was dissolved in DMSO-MeOH (1:1, 1 mL) and purified by preparative HPLC to give the title compound (0.800 mg).

MS (ESI+, m/e) 372(M+1)

### Example 396

### 2-chloro-4-{4-[(2,4-difluorobenzyl)oxy]-3,5-dimethyl-1H-pyrazol-1-yl}benzonitrile

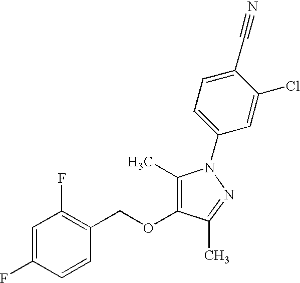

Using 1-(chloromethyl)-2,4-difluorobenzene as a starting material and in the same manner as in Example 395, the title compound was obtained.

MS (ESI+, m/e) 374(M+1).

### Example 397

### 2-chloro-4-[3,5-dimethyl-4-(pyridin-4-ylmethoxy)-1H-pyrazol-1-yl]benzonitrile trifluoroacetate

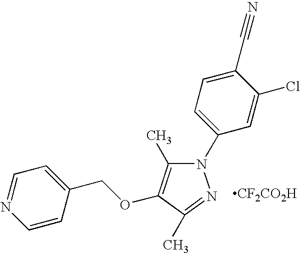

Using 4-(chloromethyl)pyridine hydrochloride and potassium carbonate (20.6 mg) as starting materials and in the same manner as in Example 395, the title compound was obtained.

MS (ESI+, m/e) 339(M+1).

### Example 398

### 2-chloro-4-[3,5-dimethyl-4-(naphthalen-1-ylmethoxy)-1H-pyrazol-1-yl]benzonitrile

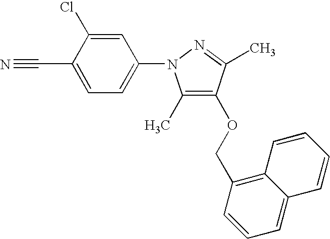

Using 1-(chloromethyl)naphthalene as a starting material and in the same manner as in Example 395, the title compound was obtained.

MS (ESI+, m/e) 388(M+1).

### Example 399

### 2-chloro-4-{4-[3-(4-fluorophenoxy)propoxy]-3,5-dimethyl-1H-pyrazol-1-yl}benzonitrile

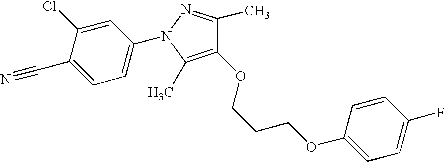

Using 1-(3-chloropropoxy)-4-fluorobenzene as a starting material and in the same manner as in Example 395, the title compound was obtained.

MS (ESI+, m/e) 400(M+1).

### Example 400

### 2-chloro-4-{3,5-dimethyl-4-[(4-nitrobenzyl)oxy]-1H-pyrazol-1-yl}benzonitrile

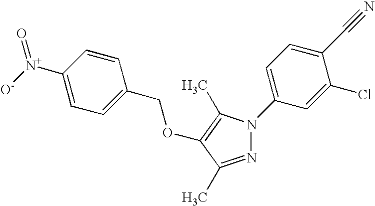

Using 1-(bromomethyl)-4-nitrobenzene as a starting material and in the same manner as in Example 395, the title compound was obtained.

MS (ESI+, m/e) 383(M+1).

### Example 401

### 2-chloro-4-{3,5-dimethyl-4-[(3-phenoxybenzyl)oxy]-1H-pyrazol-1-yl}benzonitrile

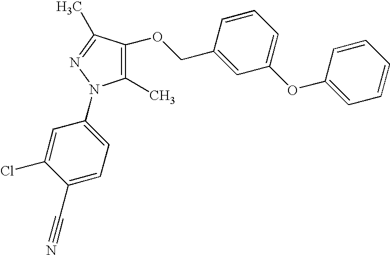

Using 1-(chloromethyl)-3-phenoxybenzene as a starting material and in the same manner as in Example 395, the title compound was obtained.

MS (ESI+, m/e) 430(M+1).

### Example 402

### 4-[4-(biphenyl-4-ylmethoxy)-3,5-dimethyl-1H-pyrazol-1-yl]-2-chlorobenzonitrile

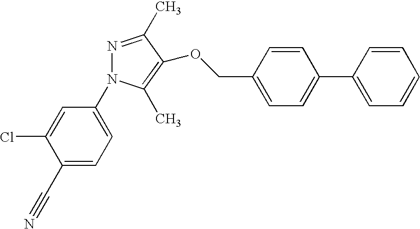

Using 4-(bromomethyl)biphenyl as a starting material and in the same manner as in Example 395, the title compound was obtained.

MS (ESI+, m/e) 414(M+1).

### Example 403

### 2-chloro-4-[3,5-dimethyl-4-(pyridin-2-ylmethoxy)-1H-pyrazol-1-yl]benzonitrile trifluoroacetate

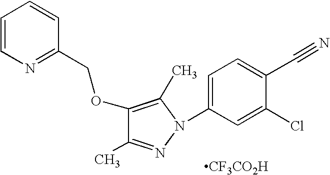

Using 2-(bromomethyl)pyridine hydrobromate and potassium carbonate (20.6 mg) as starting materials and in the same manner as in Example 395, the title compound was obtained.

MS (ESI+, m/e) 339(M+1).

### Example 404

### 2-chloro-4-[3,5-dimethyl-4-(pyridin-3-ylmethoxy)-1H-pyrazol-1-yl]benzonitrile trifluoroacetate

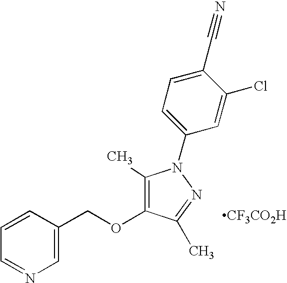

Using 3-(bromomethyl)pyridine hydrobromate and potassium carbonate (20.6 mg) as starting materials and in the same manner as in Example 395, the title compound was obtained.

MS (ESI+, m/e) 339(M+1).

### Example 405

### 2-chloro-4-(3,5-dimethyl-4-{[6-(trifluoromethyl)pyridin-3-yl]methoxy}-1H-pyrazol-1-yl)benzonitrile

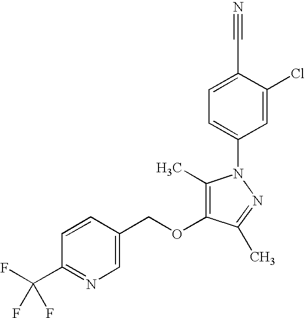

Using 5-(chloromethyl)-2-(trifluoromethyl)pyridine as a starting material and in the same manner as in Example 395, the title compound was obtained.

MS (ESI+, m/e) 407(M+1).

### Example 406

### 2-chloro-4-{3,5-dimethyl-4-[(5-methylisoxazol-3-yl)methoxy]-1H-pyrazol-1-yl}benzonitrile

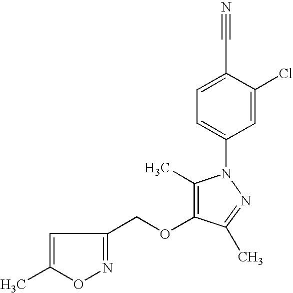

Using 3-(chloromethyl)-5-methylisoxazole as a starting material and in the same manner as in Example 395, the title compound was obtained.

MS (ESI+, m/e) 343(M+1).

### Example 407

### 2-chloro-4-{4-[(1,3-dimethyl-1H-pyrazol-5-yl)methoxy]-3,5-dimethyl-1H-pyrazol-1-yl}benzonitrile

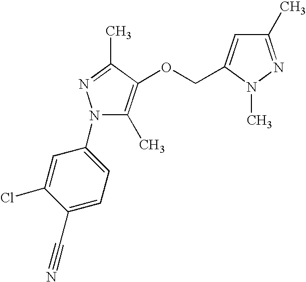

Using 5-(chloromethyl)-1,3-dimethyl-1H-pyrazole as a starting material and in the same manner as in Example 395, the title compound was obtained.

MS (ESI+, m/e) 356(M+1).

### Example 408

### 2-chloro-4-[3,5-dimethyl-4-(1-phenylethoxy)-1H-pyrazol-1-yl]benzonitrile

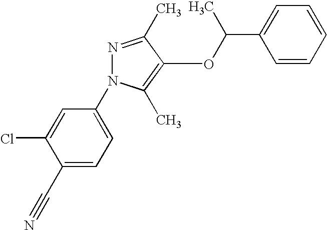

Using (1-bromoethyl)benzene as a starting material and in the same manner as in Example 395, the title compound was obtained.

MS (ESI+, m/e) 352(M+1).

### Example 409

### 2-chloro-4-[4-(imidazo[1,2-a]pyridin-5-ylmethoxy)-3,5-dimethyl-1H-pyrazol-1-yl]benzonitrile trifluoroacetate

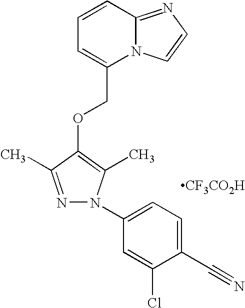

Using 5-(chloromethyl)imidazo[1,2-a]pyridine as a starting material and in the same manner as in Example 395, the title compound was obtained.

MS (ESI+, m/e) 378(M+1).

### Example 410

### 2-chloro-4-(4-{[1-(cyclopropylmethyl)-1H-imidazol-5-yl]methoxy}-3,5-dimethyl-1H-pyrazol-1-yl)benzonitrile trifluoroacetate

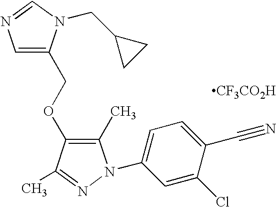

Using 5-(chloromethyl)-1-(cyclopropylmethyl)-1H-imidazole as a starting material and in the same manner as in Example 395, the title compound was obtained.

MS (ESI+, m/e) 382(M+1).

### Example 411

### 2-{[1-(3-chloro-4-cyanophenyl)-3,5-dimethyl-1H-pyrazol-4-yl]oxy}-2-phenylacetamide

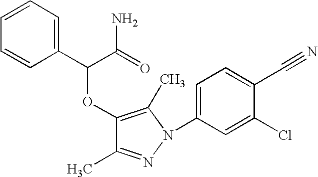

Using 2-chloro-2-phenylacetamide as a starting material and in the same manner as in Example 395, the title compound was obtained.

MS (ESI+, m/e) 381(M+1).

### Example 412

### 2-chloro-4-{3,5-dimethyl-4-[(5-oxo-5H-[1,3]thiazolo[3,2-a]pyrimidin-7-yl)methoxy]-1H-pyrazol-1-yl}benzonitrile

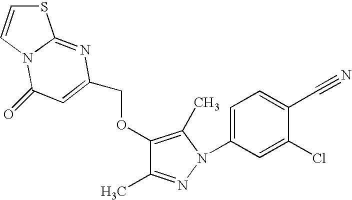

Using 7-(chloromethyl)-5H-[1,3]thiazolo[3,2-a]pyrimidin-5-one as a starting material and in the same manner as in Example 395, the title compound was obtained.

MS (ESI+, m/e) 412(M+1).

### Example 413

### 2-chloro-4-{3,5-dimethyl-4-[(4-oxo-3,4-dihydrothieno[3,2-d]pyrimidin-2-yl)methoxy]-1H-pyrazol-1-yl}benzonitrile

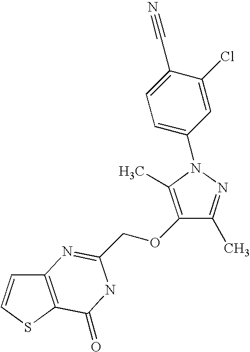

Using 2-(chloromethyl)thieno[3,2-d]pyrimidin-4(3H)-one as a starting material and in the same manner as in Example 395, the title compound was obtained.

MS (ESI+, m/e) 412(M+1).

### Example 414

### 2-{[1-(3-chloro-4-cyanophenyl)-3,5-dimethyl-1H-pyrazol-4-yl]oxy}-N-(4-methyl-1,3-thiazol-2-yl)-N-phenylacetamide

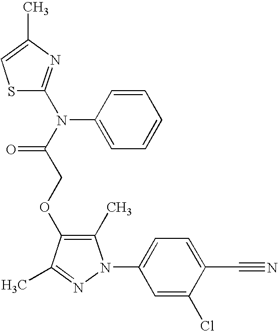

Using 2-chloro-N-(4-methyl-1,3-thiazol-2-yl)-N-phenylacetamide as a starting material and in the same manner as in Example 395, the title compound was obtained.

MS (ESI+, m/e) 478(M+1).

### Example 415

### 2-chloro-4-{3,5-dimethyl-4-[(5-methyl-4-oxo-3,4-dihydrothieno[2,3-d]pyrimidin-2-yl)methoxy]-1H-pyrazol-1-yl}benzonitrile

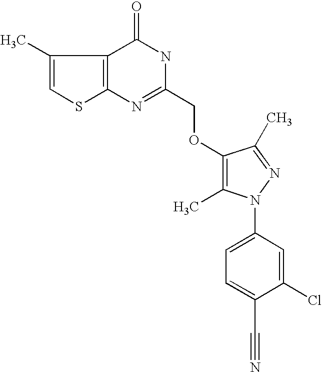

Using 2-(chloromethyl)-5-methylthieno[2,3-d]pyrimidin-4(3H)-one as a starting material and in the same manner as in Example 395, the title compound was obtained.

MS (ESI+, m/e) 426(M+1).

### Example 416

### 2-chloro-4-[4-({2-[(E)-2-(4-chlorophenyl)ethenyl]-1,3-oxazol-4-yl}methoxy)-3,5-dimethyl-1H-pyrazol-1-yl]benzonitrile

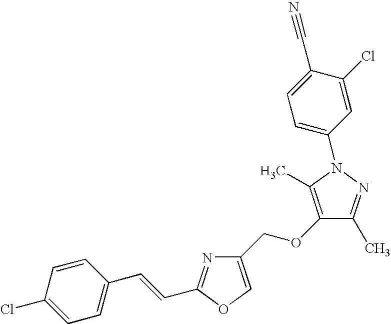

Using 4-(chloromethyl)-2-[(E)-2-(4-chlorophenyl)ethenyl]-1,3-oxazole as a starting material and in the same manner as in Example 395, the title compound was obtained.

MS (ESI+, m/e) 465(M+1).

### Example 417

### 2-chloro-4-{3,5-dimethyl-4-{(5-thiophen-3-yl-1,2,4-oxadiazol-3-yl)methoxy}-1H-pyrazol-1-yl}benzonitrile

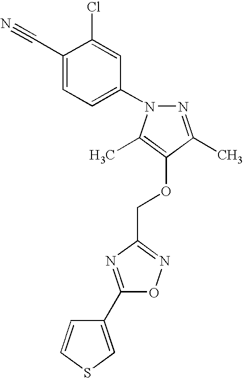

Using 3-(chloromethyl)-5-thiophen-3-yl-1,2,4-oxadiazole as a starting material and in the same manner as in Example 395, the title compound was obtained.

MS (ESI+, m/e) 412(M+1).

### Example 418

### 2-chloro-4-[4-(imidazo[1,2-a]pyrimidin-2-ylmethoxy)-3,5-dimethyl-1H-pyrazol-1-yl]benzonitrile trifluoroacetate

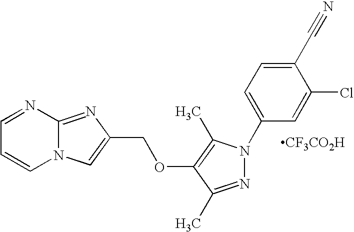

Using 2-(chloromethyl)imidazo[1,2-a]pyrimidine as a starting material and in the same manner as in Example 395, the title compound was obtained.

MS (ESI+, m/e) 379(M+1).

### Example 419

### 2-{[1-(3-chloro-4-cyanophenyl)-3,5-dimethyl-1H-pyrazol-4-yl]oxy}-N-(4-fluorophenyl)acetamide

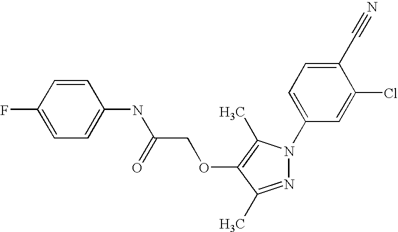

Using 2-chloro-N-(4-fluorophenyl)acetamide as a starting material and in the same manner as in Example 395, the title compound was obtained.

MS (ESI+, m/e) 399(M+1).

### Example 420

### 4-({[1-(3-chloro-4-cyanophenyl)-3,5-dimethyl-1H-pyrazol-4-yl]oxy}methyl)benzamide

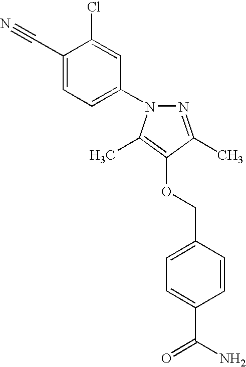

Using 4-({[1-(3-chloro-4-cyanophenyl)-3,5-dimethyl-1H-pyrazol-4-yl]oxy}methyl)benzoic acid synthesized in Example 390 as a starting material and in the same manner as in Example 385, the title compound was obtained.

¹H-NMR (CDCl₃) δ:2.24 (3H, s), 2.25 (3H, s), 4.95 (2H, s), 5.54 (1H, brs), 6.05 (1H, brs), 7.44-7.54 (3H, m), 7.67-7.74 (2H, m), 7.81-7.88 (2H, m).

### Example 421

### 2-chloro-4-{3,5-dimethyl-4-[(4-methylbenzyl)oxy]-1H-pyrazol-1-yl}benzonitrile

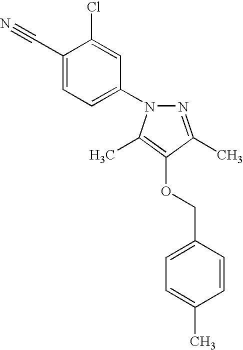

Using 1-(chloromethyl)-4-methylbenzene as a starting material and in the same manner as in Example 387, the title compound was obtained.

¹H-NMR (CDCl₃) δ:2.19 (3H, s), 2.22 (3H, s), 2.37 (3H, s), 4.84 (2H, s), 7.14-7.30 (4H, m), 7.47 (1H, dd), 7.66-7.74 (2H, m).

### Example 422

### 5-{[1-(3-chloro-4-cyanophenyl)-3,5-dimethyl-1H-pyrazol-4-yl](hydroxy)methyl}pyridine-2-carbonitrile

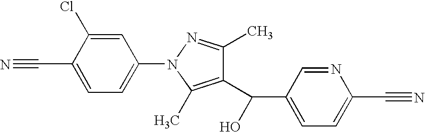

A mixture of 4-{4-[(6-bromopyridin-3-yl)(hydroxy)methyl]-3,5-dimethyl-1H-pyrazol-1-yl}-2-chlorobenzonitrile (0.115 g) synthesized in Example 392, copper(I) cyanide (0.037 g) and NMP (2.0 mL) was stirred at 150° C. for 10 min and at 180° C. for 60 min under microwave irradiation. The reaction mixture was allowed to cool and mixed with saturated aqueous sodium hydrogen carbonate, and the mixture was extracted twice with ethyl acetate. The organic layers were combined, washed with diluted aqueous ammonia and saturated brine, dried over anhydrous magnesium sulfate and concentrated under reduced pressure. The residue was purified by column chromatography (hexane-ethyl acetate), and the obtained oil was mixed with diisopropyl ether to allow solidification. Diisopropyl ether was evaporated under reduced pressure to give the title compound (0.0355 g) as a yellow solid.

¹H-NMR (CDCl₃) δ:2.17 (3H, s), 2.25 (1H, brs), 2.35 (3H, s), 6.02 (1H, d), 7.47 (1H, dd), 7.66-7.81 (3H, m), 7.94 (1H, ddd), 8.68-8.73 (1H, m).

### Example 423

### 4-{[1-(3-chloro-4-cyanophenyl)-3,5-dimethyl-1H-pyrazol-4-yl]oxy}-N-[2-(dimethylamino)-2-oxoethyl]benzamide

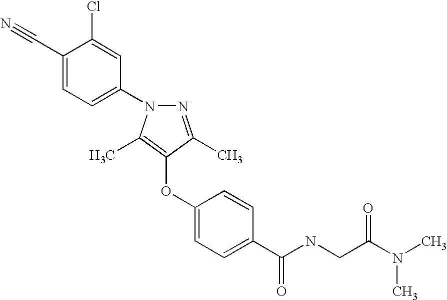

Using N-[(4-{[1-(3-chloro-4-cyanophenyl)-3,5-dimethyl-1H-pyrazol-4-yl]oxy}phenyl)carbonyl]glycine synthesized in Example 464 and dimethylamine-THF solution as starting materials and in the same manner as in Example 386, the title compound was obtained.

¹H-NMR (CDCl₃) δ:2.14 (3H, s), 2.32 (3H, s), 3.04 (3H, s), 3.05 (3H, s), 4.23 (2H, d), 6.92-6.99 (2H, m), 7.25 (1H, brs), 7.57 (1H, dd), 7.76 (1H, d), 7.79-7.86 (3H, m).

### Example 424

### 4-{[1-(3-chloro-4-cyanophenyl)-3,5-dimethyl-1H-pyrazol-4-yl]oxy}-N-(2-hydroxyethyl)-N-methylbenzamide

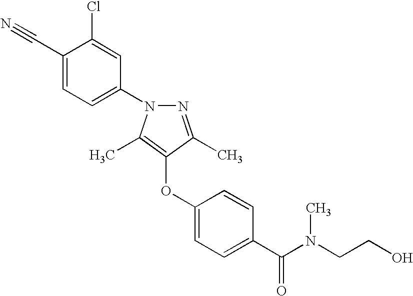

To a solution of 4-{[1-(3-chloro-4-cyanophenyl)-3,5-dimethyl-1H-pyrazol-4-yl]oxy}benzoic acid (100 mg) synthesized in Example 306 and 4-(4,6-dimethoxy-1,3,5-triazin-2-yl)-4-methylmorpholin-4-ium chloride (113 mg) in THF-2-propanol (1:1, 2 mL) was added 2-(methylamino)ethanol (0.0327 mL), and the mixture was stirred at room temperature for 17 hr. The reaction mixture was concentrated and mixed with 1 mol/l hydrochloric acid, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (ethyl acetate-methanol) and recrystallized from acetone-hexane to give the title compound (58.4 mg) as white crystals.

¹H-NMR (CDCl₃) δ:2.15 (3H, s), 2.17 (1H, s), 2.32 (3H, s), 3.11 (3H, s), 3.71 (2H, brs), 3.90 (2H, brs), 6.92-6.97 (2H, m), 7.42-7.49 (2H, m), 7.56 (1H, dd), 7.76 (1H, d), 7.80 (1H, d).

### Example 425

### N-tert-butyl-4-{[1-(3-chloro-4-cyanophenyl)-3,5-dimethyl-1H-pyrazol-4-yl]oxy}-N-methylbenzamide

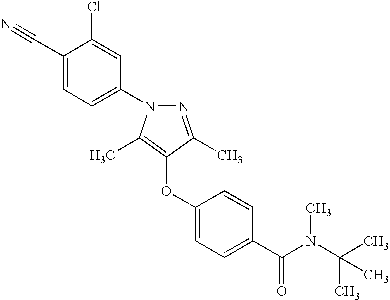

Using 4-{[1-(3-chloro-4-cyanophenyl)-3,5-dimethyl-1H-pyrazol-4-yl]oxy}benzoic acid synthesized in Example 306 and N-methyl-tert-butylamine as starting materials and in the same manner as in Example 386, the title compound was obtained.

¹H-NMR (CDCl₃) δ:1.50 (9H, s), 2.14 (3H, s), 2.31 (3H, s), 2.91 (3H, s), 6.87-6.93 (2H, m), 7.40-7.46 (2H, m), 7.56 (1H, dd), 7.76 (1H, d), 7.80 (1H, d).

### Example 426

### 4-{4-[4-(5-tert-butyl-1,3,4-oxadiazol-2-yl)phenoxy]-3,5-dimethyl-1H-pyrazol-1-yl}-2-chlorobenzonitrile

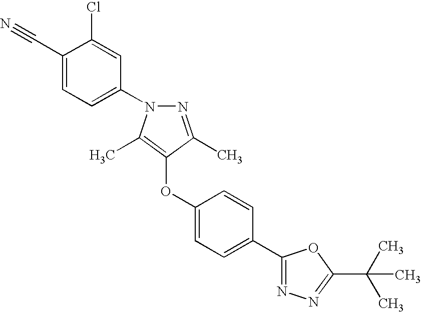

Using 2,2-dimethylpropanoic acid as a starting material and in the same manner as in Example 394, the title compound was obtained.

¹H-NMR (CDCl₃) δ:1.48 (9H, s), 2.15 (3H, s), 2.34 (3H, s), 7.00-7.06 (2H, m), 7.57 (1H, dd), 7.77 (1H, d), 7.81 (1H, d), 7.97-8.03 (2H, m).

### Example 427

### 4-({[1-(3-chloro-4-cyanophenyl)-3,5-dimethyl-1H-pyrazol-4-yl]oxy}methyl)-N,N-dimethylbenzamide

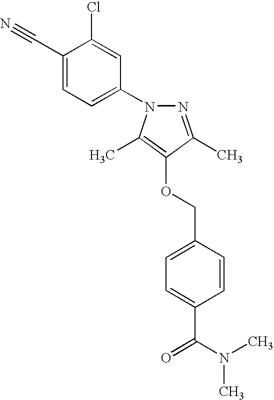

Using 4-({[1-(3-chloro-4-cyanophenyl)-3,5-dimethyl-1H-pyrazol-4-yl]oxy}methyl)benzoic acid synthesized in Example 390 and dimethylamine-THF solution as starting materials and in the same manner as in Example 386, the title compound was obtained.

¹H-NMR (CDCl₃) δ:2.25 (6H, s), 2.99 (3H, brs), 3.13 (3H, brs), 4.91 (2H, s), 7.45 (4H, s), 7.46-7.50 (1H, m), 7.69-7.74 (2H, m).

### Example 428

### methyl 5-(4-{[1-(3-chloro-4-cyanophenyl)-3,5-dimethyl-1H-pyrazol-4-yl]oxy}phenyl)-1,3,4-oxadiazole-2-carboxylate

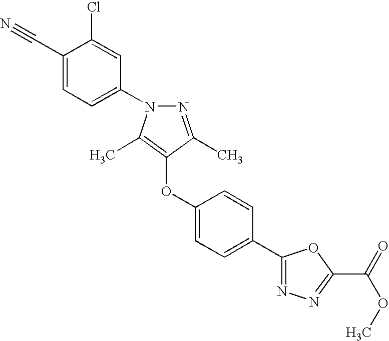

A solution of 4-{[1-(3-chloro-4-cyanophenyl)-3,5-dimethyl-1H-pyrazol-4-yl]oxy}benzohydrazide (200 mg) synthesized in Example 465, methyl chloroglyoxylate (0.0724 mL) and triethylamine (0.365 mL) in THF (1 mL) was stirred at room temperature for 4 hr. 4-Methylbenzenesulfonyl chloride (300 mg) was added to the reaction mixture, and the mixture was stirred at room temperature for 19 hr and at 50° C. for 4 hr. Triethylamine (0.365 mL) and 4-methylbenzenesulfonyl chloride (300 mg) were added to the reaction mixture, and the mixture was heated at 50° C. for 5 hr. The reaction mixture was allowed to cool and mixed with saturated aqueous sodium hydrogen carbonate solution, and the mixture was extracted with ethyl acetate. The ethyl acetate layer was washed with saturated aqueous sodium hydrogen carbonate solution and saturated brine, dried over anhydrous sodium sulfate and concentrated. The residue was purified by column chromatography (hexane-ethyl acetate) and recrystallized from ethyl acetate-hexane to give the title compound (14.7 mg) as white crystals.

¹H-NMR (CDCl₃) δ:2.16 (3H, s), 2.34 (3H, s), 4.09 (3H, s), 7.05-7.11 (2H, m), 7.58 (1H, dd), 7.78 (1H, d), 7.81 (1H, d), 8.11-8.17 (2H, m).

### Example 429

### 2-chloro-4-[4-(4-{5-[(dimethylamino)methyl]-1,3,4-oxadiazol-2-yl}phenoxy)-3,5-dimethyl-1H-pyrazol-1-yl]benzonitrile

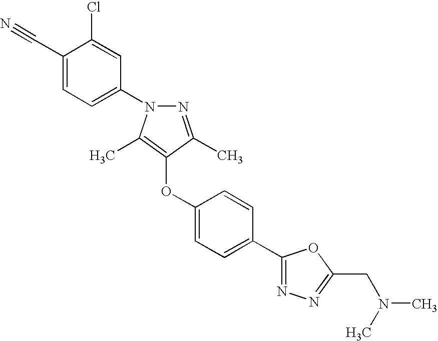

Using N,N-dimethylglycine as a starting material and in the same manner as in Example 394, the title compound was obtained.

¹H-NMR (CDCl₃) δ:2.15 (3H, s), 2.34 (3H, s), 2.40 (6H, s), 3.82 (2H, s), 7.00-7.07 (2H, m), 7.57 (1H, dd), 7.77 (1H, d), 7.81 (1H, d), 8.00-8.08 (2H, m).

### Example 430

### 2-chloro-4-[3,5-dimethyl-4-(4-{5-[(methylsulfonyl)methyl]-1,3,4-oxadiazol-2-yl}phenoxy)-1H-pyrazol-1-yl]benzonitrile

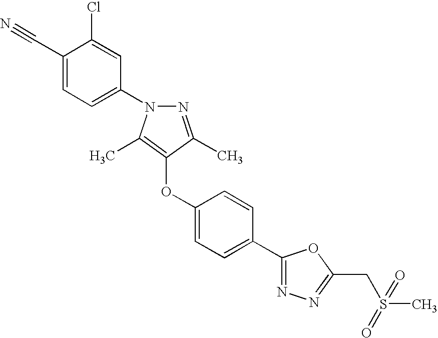

Using (methylsulfonyl)acetic acid as a starting material and in the same manner as in Example 394, the title compound was obtained.

¹H-NMR (CDCl₃) δ:2.16 (3H, s), 2.34 (3H, s), 3.15 (3H, s), 4.62 (2H, d), 7.04-7.10 (2H, m), 7.57 (1H, dd), 7.77 (1H, d), 7.81 (1H, d), 8.01-8.08 (2H, m).

### Example 431

### 2-chloro-4-{3,5-dimethyl-4-[4-(2-methylimidazo[5,1-b][1,3,4]oxadiazol-5-yl)phenoxy]-1H-pyrazol-1-yl}benzonitrile

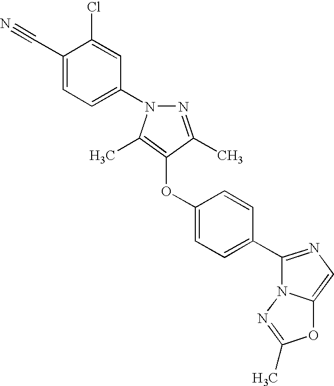

A solution of N-[2-(2-acetylhydrazino)-2-oxoethyl]-4-{[1-(3-chloro-4-cyanophenyl)-3,5-dimethyl-1H-pyrazol-4-yl]oxy}benzamide (123 mg) synthesized in Example 466 in phosphorus oxychloride (10 mL) was stirred at 100° C. for 1 hr, and the mixture was concentrated. The residue was mixed with saturated aqueous sodium hydrogen carbonate solution, and the mixture was extracted with ethyl acetate. The ethyl acetate layer was washed with saturated aqueous sodium hydrogen carbonate solution and saturated brine, dried over anhydrous sodium sulfate and concentrated. The residue was purified by column chromatography (hexane-ethyl acetate) and recrystallized from ethyl acetate-hexane to give the title compound (21.5 mg) as yellow crystals.

¹H-NMR (CDCl₃) δ:2.16 (3H, s), 2.34 (3H, s), 2.58 (3H, s), 6.61 (1H, s), 6.96-7.03 (2H, m), 7.57 (1H, dd), 7.76 (1H, d), 7.81 (1H, d), 8.06-8.13 (2H, m).

### Example 432

### 2-chloro-4-(4-{4-[5-(1-hydroxy-1-methylethyl)-1,3,4-oxadiazol-2-yl]phenoxy}-3,5-dimethyl-1H-pyrazol-1-yl)benzonitrile

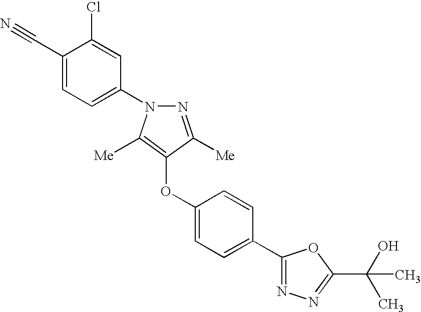

Using methyl 5-(4-{[1-(3-chloro-4-cyanophenyl)-3,5-dimethyl-1H-pyrazol-4-yl]oxy}phenyl)-1,3,4-oxadiazole-2-carboxylate synthesized in Example 428 as a starting material and in the same manner as in Example 379, the title compound was obtained.

¹H-NMR (CDCl₃) δ:1.78 (6H, s), 2.16 (3H, s), 2.35 (3H, s), 2.62 (1H, brs), 7.01-7.09 (2H, m), 7.59 (1H, dd), 7.78 (1H, d), 7.82 (1H, d), 7.99-8.07 (2H, m).

### Example 433

### 2-chloro-4-(4-{hydroxy[6-(1H-pyrazol-1-yl)pyridin-3-yl]methyl}-3,5-dimethyl-1H-pyrazol-1-yl)benzonitrile

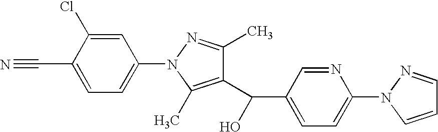

To a mixture of 4-{4-[(6-bromopyridin-3-yl)(hydroxy)methyl]-3,5-dimethyl-1H-pyrazol-1-yl}-2-chlorobenzonitrile (0.120 g) synthesized in Example 392, copper(I) iodide (0.0055 g), 1H-pyrazole (0.0392 g), N,N′-dimethylethylenediamine (0.0062 mL), tripotassium phosphate (0.122 g) and toluene (1.0 mL) was added 6 drops of NMP with a Pasteur pipette, and the mixture was stirred at 130° C. for 20 min under microwave irradiation. The reaction mixture was allowed to cool and mixed with saturated aqueous sodium hydrogen carbonate, and the mixture was extracted with ethyl acetate. The organic layer was washed with saturated brine, dried over anhydrous magnesium sulfate and concentrated under reduced pressure. The residue was purified twice by column chromatography (hexane-ethyl acetate) to give the title compound (0.0072 g) as an amorphous solid.

¹H-NMR (CDCl₃) δ:2.21 (3H, s), 2.37 (4H, m), 5.99 (1H, s), 6.48 (1H, dd), 7.47 (1H, dd), 7.67-7.79 (3H, m), 7.84 (1H, dd), 7.98 (1H, d), 8.41 (1H, d), 8.55 (1H, d).

### Example 434

### methyl 4-{[1-(3-chloro-4-cyanophenyl)-3,5-diethyl-1H-pyrazol-4-yl]methyl}benzoate

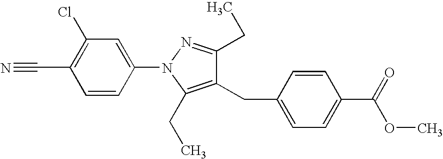

Using methyl 4-[(3,5-diethyl-1H-pyrazol-4-yl)methyl]benzoate synthesized in Reference Example 39 as a starting material and in the same manner as in Example 46, the title compound was obtained as a white solid.

¹H-NMR (CDCl₃) δ:1.03 (3H, t), 1.17 (3H, t), 2.51 (2H, q), 2.70 (2H, q), 3.88 (2H, s), 3.91 (3H, s), 7.21 (2H, d), 7.51 (1H, dd), 7.72-7.79 (2H, m), 7.96 (2H, d).

### Example 435

### 2-chloro-4-(4-{4-[5-(2-methoxyethyl)-1,3,4-oxadiazol-2-yl]phenoxy}-3,5-dimethyl-1H-pyrazol-1-yl)benzonitrile

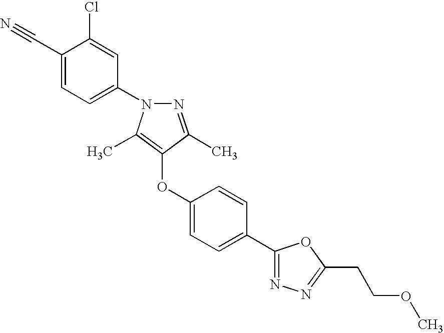

Using 3-methoxypropanoic acid as a starting material and in the same manner as in Example 394, the title compound was obtained.

¹H-NMR (CDCl₃) δ:2.14 (3H, s), 2.33 (3H, s), 3.19 (2H, t), 3.39 (3H, s), 3.84 (2H, t), 6.98-7.07 (2H, m), 7.56 (1H, dd), 7.76 (1H, d), 7.80 (1H, d), 7.95-8.03 (2H, m).

### Example 436

### 4-{[1-(3-chloro-4-cyanophenyl)-3,5-diethyl-1H-pyrazol-4-yl]methyl}benzoic acid

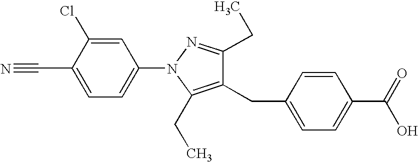

Methyl 4-{[1-(3-chloro-4-cyanophenyl)-3,5-diethyl-1H-pyrazol-4-yl]methyl}benzoate (2.32 g) synthesized in Example 434 was suspended in a mixed solvent of THF (23 mL)-methanol (5 mL), and the suspension was dissolved by heating to 40° C. An aqueous solution (5.0 mL) of lithium hydroxide monohydrate (0.358 g) was added dropwise to the solution, and the mixture was stirred at 40° C. for 3 hr. The reaction mixture was cooled to room temperature and concentrated under reduced pressure and extracted with ethyl acetate. The organic layer was washed with saturated brine, dried over anhydrous magnesium sulfate and concentrated under reduced pressure. The residue was purified by column chromatography (hexane-ethyl acetate) to give the title compound (1.95 g) as a white solid.

¹H-NMR (CDCl₃) δ:1.04 (3H, t), 1.18 (3H, t), 1.61 (1H, brs), 2.52 (2H, q), 2.71 (2H, q), 3.90 (2H, s), 7.21-7.25 (2H, m), 7.52 (1H, dd), 7.70-7.82 (2H, m), 8.02 (2H, d).

### Example 437

### 4-{[1-(3-chloro-4-cyanophenyl)-3,5-diethyl-1H-pyrazol-4-yl]methyl}-N-(2-hydroxy-2-methylpropyl)benzamide

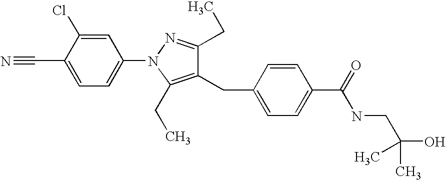

Using 4-{[1-(3-chloro-4-cyanophenyl)-3,5-diethyl-1H-pyrazol-4-yl]methyl}benzoic acid synthesized in Example 436 as a starting material and in the same manner as in Example 356, the title compound was obtained as a white solid.

¹H-NMR (CDCl₃) δ:1.04 (3H, t), 1.18 (3H, t), 1.29 (6H, s), 2.16 (2H, s), 2.52 (2H, q), 2.70 (2H, q), 3.48 (2H, d), 3.88 (2H, s), 7.21 (2H, d), 7.51 (1H, dd), 7.69-7.78 (4H, m).

### Example 438

### 2-chloro-4-[3,5-dimethyl-4-(4-{5-[(methylsulfonyl)methyl]-1,3,4-oxadiazol-2-yl}benzyl)-1H-pyrazol-1-yl]benzonitrile

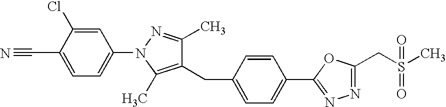

A mixture of 4-{[1-(3-chloro-4-cyanophenyl)-3,5-dimethyl-1H-pyrazol-4-yl]methyl}benzoic acid (10.0 g) obtained in Example 92, tert-butyl carbazate (7.23 g), 4-(4,6-dimethoxy-1,3,5-triazin-2-yl)-4-methylmorpholin-4-ium chloride (9.07 g) and DMF (100 mL) was stirred at room temperature for 3 days. Water was added dropwise slowly to the reaction mixture with stirring, and the resulting solid was collected by filtration. The solid was washed with water and ethyl acetate and dried under reduced pressure to give a white solid (9.84 g). A part (0.300 g) of the obtained solid was mixed with phosphoryl chloride (10.0 mL), methanesulfonylacetic acid (2.16 g) was added, and the mixture was stirred at 100° C. for 45 min. The mixture was cooled to room temperature and concentrated under reduced pressure. The residue was neutralized with saturated aqueous sodium hydrogen carbonate and extracted with ethyl acetate. The organic layer was washed with saturated brine, dried over anhydrous magnesium sulfate and concentrated under reduced pressure. The residue was purified by column chromatography (hexane-ethyl acetate) and fractions containing the title compound were collected and concentrated. The residue was dissolved in ethyl acetate, washed twice with saturated aqueous sodium hydrogen carbonate and once with saturated brine, dried over anhydrous magnesium sulfate and concentrated under reduced pressure. The residue was recrystallized from ethyl acetate-hexane to give the title compound (0.115 g) as a white solid.

¹H-NMR (CDCl₃) δ:2.19 (3H, s), 2.34 (3H, s), 3.15 (3H, s), 3.88 (2H, s), 4.62 (2H, d), 7.30 (2H, d), 7.52 (1H, dd), 7.72-7.78 (2H, m), 7.97-8.03 (2H, m).

### Example 439

### 2-chloro-4-(3,5-dimethyl-4-{[3-(trifluoromethyl)-1H-pyrazol-5-yl]methoxy}-1H-pyrazol-1-yl)benzonitrile

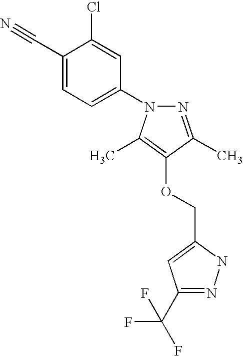

Using 5-(iodomethyl)-3-(trifluoromethyl)-1-trityl-1H-pyrazole as a starting material and in the same manner as in Example 387, 2-chloro-4-(3,5-dimethyl-4-{[3-(trifluoromethyl)-1-trityl-1H-pyrazol-5-yl]methoxy}-1H-pyrazol-1-yl)benzonitrile was obtained. A solution of the obtained compound (356 mg) in 4 mol/l hydrochloric acid-ethyl acetate (3 mL) was stirred at room temperature for 4 hr, and the reaction mixture was concentrated. The residue was purified by column chromatography (hexane-ethyl acetate) and recrystallized from ethyl acetate-heptane to give the title compound (49.7 mg) as yellow crystals.

¹H-NMR (CDCl₃) δ:2.23 (3H, s), 2.24 (3H, s), 4.99 (2H, s), 6.55 (1H, s), 7.47 (1H, dd), 7.70-7.76 (2H, m), 10.49 (1H, brs).

### Example 440

### 4-{4-[(5-bromopyridin-2-yl)(hydroxy)methyl]-3,5-dimethyl-1H-pyrazol-1-yl}-2-chlorobenzonitrile

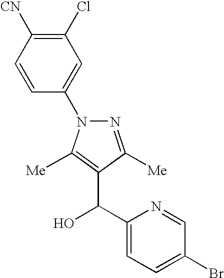

2,5-Dibromopyridine (0.909 g) was suspended in toluene (50.0 mL), and the suspension was cooled to −78° C. in a hexane-dry ice bath. n-Butyllithium-hexane solution (1.60M, 2.88 mL) was added to the suspension, and the mixture was stirred at −78° C. for 2 hr. A suspension (5.0 mL) of 2-chloro-4-(4-formyl-3,5-dimethyl-1H-pyrazol-1-yl)benzonitrile (0.830 g) synthesized in Reference Example 17 in toluene was added to the mixture, and the mixture was stirred at −78° C. for 1 hr. The reaction mixture was removed from the hexane-dry ice bath, and stirred for 1 hr while allowing to warm to room temperature. A saturated aqueous ammonium chloride solution was added to the reaction mixture under ice-cooling, and the mixture was extracted twice with ethyl acetate. The organic layers were combined, washed with saturated brine, dried over anhydrous magnesium sulfate and concentrated under reduced pressure. The residue was purified by column chromatography (hexane-ethyl acetate) to give the title compound (0.195 g) as a pale-yellow oil.

¹H-NMR (CDCl₃) δ:2.10 (3H, s), 2.33 (3H, s), 4.85 (1H, brs), 5.77 (1H, s), 7.08 (1H, d), 7.47 (1H, dd), 7.69 (1H, d), 7.74 (1H, d), 7.82 (1H, dd), 8.67 (1H, d).

### Example 441

### 2-chloro-4-(4-{hydroxy[5-(2-oxopyrrolidin-1-yl)pyridin-2-yl]methyl}-3,5-dimethyl-1H-pyrazol-1-yl)benzonitrile

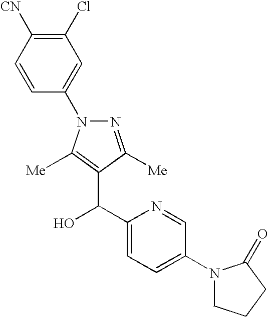

To a mixture of 4-{4-[(5-bromopyridin-2-yl)(hydroxy)methyl]-3,5-dimethyl-1H-pyrazol-1-yl}-2-chlorobenzonitrile (0.158 g) synthesized in Example 440, 2-pyrrolidone (0.0374 mL), tripotassium phosphate (0.160 g), N,N′-dimethylethylenediamine (0.0040 g), copper(I) iodide (0.00432 g) and xylene (1.0 mL) was added 1 drop of NMP with a Pasteur pipette, and the mixture was stirred at 130° C. for 20 min, at 160° C. for 20 min and at 180° C. for 20 min under microwave irradiation. The reaction mixture was allowed to cool, saturated aqueous sodium hydrogen carbonate was added, and the mixture was extracted with ethyl acetate. The organic layer was washed with saturated brine, dried over anhydrous magnesium sulfate and concentrated under reduced pressure. The residue was purified twice by column chromatography (hexane-ethyl acetate, and then methanol-ethyl acetate) to give the title compound (0.0130 g) as a pale-yellow oil.

¹H-NMR (CDCl₃) δ:2.09 (3H, s), 2.18-2.31 (2H, m), 2.33 (3H, s), 2.65 (2H, t), 3.92 (2H, t), 5.16 (1H, brs), 5.79 (1H, s), 7.13 (1H, d), 7.48 (1H, dd), 7.67-7.77 (2H, m), 8.24 (1H, dd), 8.73 (1H, d).

### Example 442

### 4-{[1-(3-chloro-4-cyanophenyl)-3,5-diethyl-1H-pyrazol-4-yl]methyl}benzamide

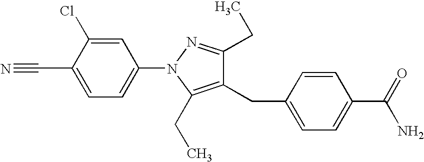

A mixture of 4-{[1-(3-chloro-4-cyanophenyl)-3,5-diethyl-1H-pyrazol-4-yl]methyl}benzoic acid (0.100 g) synthesized in Example 436, 1-hydroxybenzotriazole ammonium salt (0.0758 g), N-(3-dimethylaminopropyl)-N′-ethylcarbodiimide hydrochloride (0.0585 g) and DMF (1.0 mL) was stirred at room temperature for 1 day. 1 mol/l Hydrochloric acid was added to the reaction mixture, and the mixture was extracted with ethyl acetate. The organic layer was washed with saturated brine, dried over anhydrous sodium sulfate and concentrated under reduced pressure. The residue was purified by column chromatography (ethyl acetate-hexane) and recrystallized from hexane-ethyl acetate to give the title compound (0.0795 g) as a white solid.

¹H-NMR (CDCl₃) δ:1.04 (3H, t) 1.18 (3H, t) 2.52 (2H, q) 2.70 (2H, q) 3.88 (2H, s) 5.55 (1H, brs) 5.97 (1H, brs) 7.22 (2H, d) 7.51 (1H, dd) 7.74 (4H, dd).

### Example 443

### 4-{[1-(3-chloro-4-cyanophenyl)-3,5-diethyl-1H-pyrazol-4-yl]methyl}-N-cyclopropylbenzamide

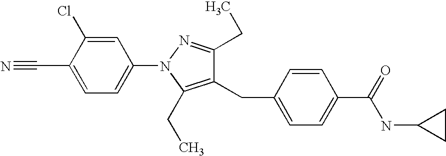

A mixture of 4-{[1-(3-chloro-4-cyanophenyl)-3,5-diethyl-1H-pyrazol-4-yl]methyl}benzoic acid (0.100 g) synthesized in Example 436, cyclopropylamine (0.0176 mL), N-(3-dimethylaminopropyl)-N′-ethylcarbodiimide hydrochloride (0.0585 g), 1-hydroxybenzotriazole (0.0343 g) and DMF (1.0 mL) was stirred at room temperature for 1 day. Cyclopropylamine (0.0176 mL) was added to the reaction mixture, and the mixture was further stirred at room temperature for 1 day. 1 mol/l Hydrochloric acid was added to the reaction mixture, and the mixture was extracted with ethyl acetate. The organic layer was washed with saturated brine, dried over anhydrous magnesium sulfate and concentrated under reduced pressure. The residue was purified by column chromatography (hexane-ethyl acetate) and recrystallized from ethyl acetate-hexane to give the title compound (0.0850 g) as a white solid.

¹H-NMR (CDCl₃) δ:0.61 (2H, dd), 0.81-0.92 (2H, m), 1.03 (3H, t), 1.17 (3H, t), 2.50 (2H, q), 2.69 (2H, q), 2.89 (1H, dd), 3.86 (2H, s), 6.17 (1H, brs), 7.18 (2H, d), 7.51 (1H, dd), 7.66 (2H, d), 7.71-7.79 (2H, m).

### Example 444

### methyl [5-(4-{[1-(3-chloro-4-cyanophenyl)-3,5-dimethyl-1H-pyrazol-4-yl]oxy}phenyl)-1,3,4-oxadiazol-2-yl]acetate

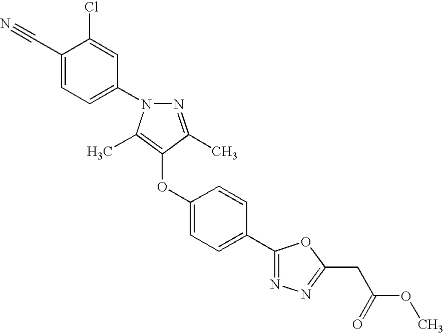

Using methyl 3-chloro-3-oxopropanoate as a starting material and in the same manner as in Example 394, the title compound was obtained.

¹H-NMR (CDCl₃) δ:2.16 (3H, s), 2.34 (3H, s), 3.79 (3H, s), 4.05 (2H, s), 7.01-7.08 (2H, m), 7.58 (1H, dd), 7.77 (1H, d), 7.81 (1H, d), 7.98-8.05 (2H, m).

### Example 445

### 4-{[1-(3-chloro-4-cyanophenyl)-3,5-dimethyl-1H-pyrazol-4-yl]methyl}-N-(2-hydroxy-2-methylpropyl)benzamide p-toluenesulfonate

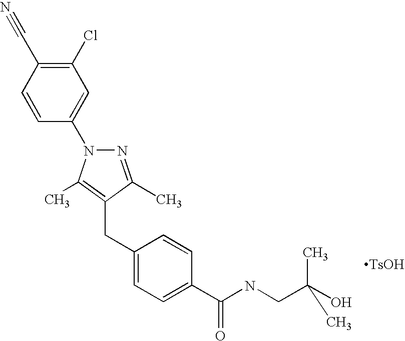

A suspension (50 mL) of 4-{[1-(3-chloro-4-cyanophenyl)-3,5-dimethyl-1H-pyrazol-4-yl]methyl}-N-(2-hydroxy-2-methylpropyl)benzamide (10.00 g) obtained in Example 356 and p-toluenesulfonic acid monohydrate (4.57 g) in acetone-water (9:1) was heated to 50° C. to dissolve the contents completely. Ethyl acetate (80 mL) was added dropwise, and the mixture was stirred at 50° C. for 30 min. After cooling to 0° C., the insoluble material was collected by filtration to give the title compound (11.98 g) as a colorless solid. The title compound obtained above and the title compound separately synthesized were combined (15.5 g), and recrystallized from acetone-water (9:1)-ethyl acetate to give the title compound (12.80 g) as colorless crystals.

melting point 162-163° C.

¹H-NMR (DMSO-d₆) δ:1.09 (6H, s), 2.11 (3H, s), 2.29 (3H, s), 2.40 (3H, s), 3.24 (2H, d), 3.84 (2H, s), 6.20 (2H, brs.), 7.12 (2H, d), 7.26 (2H, d), 7.44-7.51 (1H, m), 7.48 (1H, d), 7.72-7.81 (3H, m), 7.95 (1H, d), 8.08 (1H, d), 8.16 (1H, t).

### Example 446

### 5-{[1-(3-chloro-4-cyanophenyl)-3,5-dimethyl-1H-pyrazol-4-yl]oxy}pyridine-2-carbonitrile

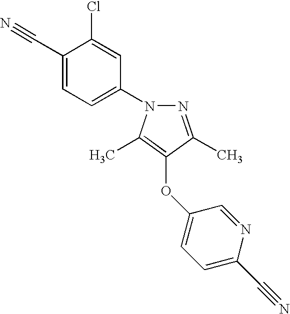

A solution of 2-chloro-4-(4-hydroxy-3,5-dimethyl-1H-pyrazol-1-yl)benzonitrile (100 mg) synthesized in Reference Example 18, 5-bromopyridine-2-carbonitrile (111 mg) and cesium carbonate (197 mg) in DMF (2 mL) was stirred at 100° C. for 3 hr. The reaction mixture was allowed to cool and mixed with saturated aqueous ammonium chloride solution, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from acetate-heptane to give the title compound (50.3 mg) as white crystals.

¹H-NMR (CDCl₃) δ:2.15 (3H, s), 2.33 (3H, s), 7.26 (1H, dd), 7.55 (1H, dd), 7.67 (1H, d), 7.74-7.82 (2H, m), 8.49 (1H, d).

### Example 447

### 2-chloro-4-{3,5-dimethyl-4-[(5-nitropyridin-2-yl)oxy]-1H-pyrazol-1-yl }benzonitrile

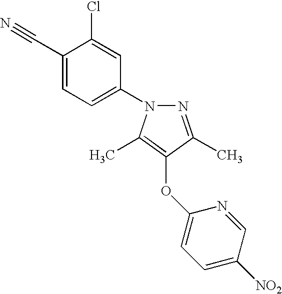

Using 2-bromo-5-nitropyridine as a starting material and in the same manner as in Example 446, the title compound was obtained.

¹H-NMR (CDCl₃) δ:2.16 (3H, s), 2.32 (3H, s), 7.15 (1H, d), 7.57 (1H, dd), 7.76 (1H, d), 7.80 (1H, d), 8.53 (1H, dd), 9.05 (1H, d).

### Example 448

### 4-{4-[(5-aminopyridin-2-yl)oxy]-3,5-dimethyl-1H-pyrazol-1-yl}-2-chlorobenzonitrile

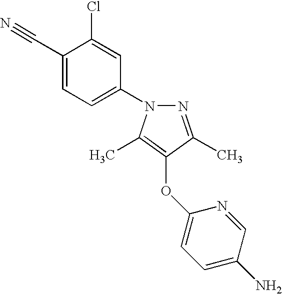

To a solution of 2-chloro-4-{3,5-dimethyl-4-[(5-nitropyridin-2-yl)oxy]-1H-pyrazol-1-yl}benzonitrile (50.0 mg) synthesized in Example 447 in THF (1 mL) were added zinc powder (89.0 mg), concentrated hydrochloric acid (0.0230 mL) and acetic acid (0.500 mL) at 0° C., and the mixture was stirred at room temperature for 4 hr. The reaction mixture was ice-cooled and mixed with 25% aqueous ammonia, and the mixture was extracted with ethyl acetate. The ethyl acetate layer was washed with saturated brine, dried over anhydrous sodium sulfate and concentrated. The residue was mixed with acetone-toluene and filtered, and the filtrate was purified by column chromatography (hexane-ethyl acetate) and recrystallized from tert-butyl methyl ether-heptane to give the title compound (32.9 mg) as yellow crystals.

¹H-NMR (CDCl₃) δ:2.13 (3H, s), 2.33 (3H, s), 3.49 (2H, brs), 6.79 (1H, dd), 7.11 (1H, dd), 7.56 (1H, dd), 7.64 (1H, dd), 7.72 (1H, d), 7.79 (1H, d).

### Example 449

### 4-({[1-(3-chloro-4-cyanophenyl)-3,5-dimethyl-1H-pyrazol-4-yl]oxy}methyl)-1H-pyrazole-5-carboxamide

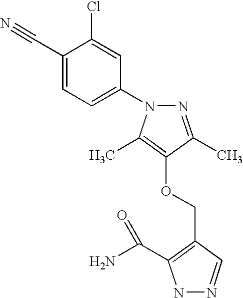

Using 4-({[1-(3-chloro-4-cyanophenyl)-3,5-dimethyl-1H-pyrazol-4-yl]oxy}methyl)-1-trityl-1H-pyrazole-5-carboxylic acid synthesized in Example 467 as a starting material and in the same manner as in Example 385, 4-({[1-(3-chloro-4-cyanophenyl)-3,5-dimethyl-1H-pyrazol-4-yl]oxy}methyl)-1-trityl-1H-pyrazole-5-carboxamide was obtained. A solution of the obtained compound (182 mg) in 4 mol/l hydrochloric acid-ethyl acetate (5 mL) was stirred at room temperature for 2 hr. The reaction mixture was concentrated and mixed with 25% aqueous ammonia, and the mixture was extracted with ethyl acetate. The ethyl acetate layer was washed with saturated aqueous sodium hydrogen carbonate solution and saturated brine, dried over anhydrous sodium sulfate and concentrated. The residue was purified by column chromatography (ethyl acetate-methanol) and recrystallized from ethyl acetate-heptane to give the title compound (42.3 mg) as yellow crystals.

¹H-NMR (DMSO-d₆) δ:2.13 (3H, s), 2.29 (3H, s), 5.07 (2H, s), 7.17 (1H, brs), 7.45 (1H, brs), 7.70 (1H, dd), 7.87-7.93 (2H, m), 8.06 (1H, d), 13.20 (1H, brs).

### Example 450

### 2-chloro-4-{3,5-dimethyl-4-[(6-nitropyridin-3-yl)oxy]-1H-pyrazol-1-yl}benzonitrile

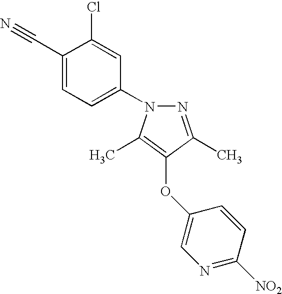

Using 5-bromo-2-nitropyridine as a starting material and in the same manner as in Example 446, the title compound was obtained.

¹H-NMR (CDCl₃) δ:2.17 (3H, s), 2.34 (3H, s), 7.43 (1H, dd), 7.56 (1H, dd), 7.77-7.82 (2H, m), 8.28 (1H, dd), 8.37 (1H, dd).

### Example 451

### 4-{4-[(6-aminopyridin-3-yl)oxy]-3,5-dimethyl-1H-pyrazol-1-yl}-2-chlorobenzonitrile

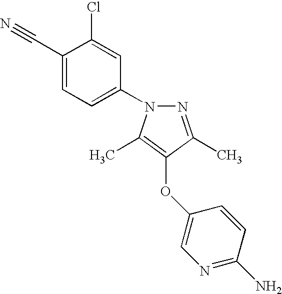

Using 2-chloro-4-{3,5-dimethyl-4-[(6-nitropyridin-3-yl)oxy]-1H-pyrazol-1-yl}benzonitrile synthesized in Example 450 as a starting material and in the same manner as in Example 448, the title compound was obtained.

¹H-NMR (CDCl₃) δ:2.15 (3H, s), 2.33 (3H, s), 4.40 (2H, brs), 6.50 (1H, dd), 7.13 (1H, dd), 7.54 (1H, dd), 7.72-7.79 (3H, m).

### Example 452

### 4-({[1-(3-chloro-4-cyanophenyl)-3,5-dimethyl-1H-pyrazol-4-yl]oxy}methyl)-N-(2-hydroxy-2-methylpropyl)-1H-pyrazole-5-carboxamide

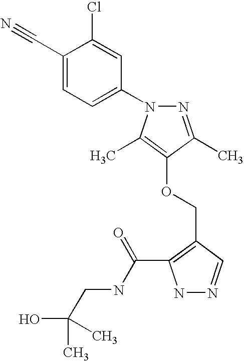

Using 4-({[1-(3-chloro-4-cyanophenyl)-3,5-dimethyl-1H-pyrazol-4-yl]oxy}methyl)-1-trityl-1H-pyrazole-5-carboxylic acid synthesized in Example 467 and 1-amino-2-methyl-2-propanol as starting materials and in the same manner as in Example 424, 4-({[1-(3-chloro-4-cyanophenyl)-3,5-dimethyl-1H-pyrazol-4-yl]oxy}methyl)-N-(2-hydroxy-2-methylpropyl)-1-trityl-1H-pyrazole-5-carboxamide was obtained. A solution of the obtained compound (220 mg) in 4 mol/l hydrochloric acid-ethyl acetate (5 mL) was stirred at room temperature for 3 hr. The reaction mixture was concentrated and mixed with 25% aqueous ammonia, and the mixture was extracted with ethyl acetate. The ethyl acetate layer was washed with saturated aqueous sodium hydrogen carbonate solution and saturated brine, dried over anhydrous sodium sulfate and concentrated. The residue was purified by column chromatography (ethyl acetate-methanol) to give the title compound (34.2 mg) as yellow crystals.

¹H-NMR (DMSO-d₆) δ:1.07 (6H, s), 2.11 (3H, s), 2.28 (3H, s), 3.18 (2H, d), 4.65 (1H, brs), 5.08 (2H, s), 7.66-7.74 (2H, m), 7.88 (1H, d), 7.93 (1H, s), 8.06 (1H, d).

### Example 453

### N-(6-{[1-(3-chloro-4-cyanophenyl)-3,5-dimethyl-1H-pyrazol-4-yl]oxy}pyridin-3-yl)acetamide

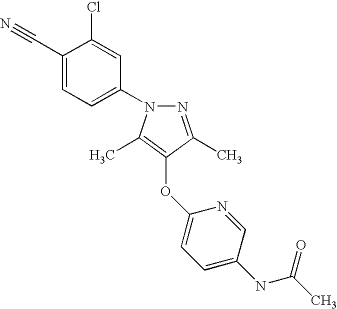

To a solution of 4-{4-[(5-aminopyridin-2-yl)oxy]-3,5-dimethyl-1H-pyrazol-1-yl}-2-chlorobenzonitrile (87.0 mg) synthesized in Example 448 in pyridine (1 mL) was added acetic anhydride (0.0363 mL), and the mixture was stirred at room temperature for 16 hr. The reaction mixture was ice-cooled and mixed with 1 mol/l hydrochloric acid, and the mixture was extracted with ethyl acetate. The ethyl acetate layer was washed with 1 mol/l hydrochloric acid, saturated aqueous sodium hydrogen carbonate solution and saturated brine, dried over anhydrous sodium sulfate and concentrated. The residue was purified by column chromatography (hexane-ethyl acetate) and recrystallized from ethyl acetate-heptane to give the title compound (68.3 mg) as pale-pink crystals.

¹H-NMR (CDCl₃) δ:2.14 (3H, s), 2.20 (3H, s), 2.32 (3H, s), 6.97 (1H, dd), 7.20 (1H, brs), 7.57 (1H, dd), 7.74 (1H, d), 7.80 (1H, d), 8.06-8.16 (2H, m).

### Example 454

### N-(5-{[1-(3-chloro-4-cyanophenyl)-3,5-dimethyl-1H-pyrazol-4-yl]oxy}pyridin-2-yl)acetamide

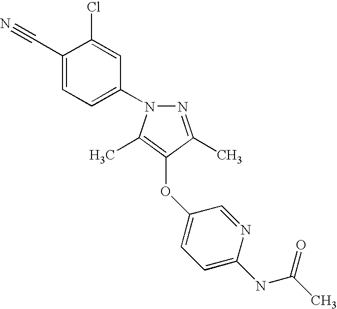

Using 4-{4-[(6-aminopyridin-3-yl)oxy]-3,5-dimethyl-1H-pyrazol-1-yl]-2-chlorobenzonitrile synthesized in Example 451 as a starting material and in the same manner as in Example 453, the title compound was obtained.

¹H-NMR (CDCl₃) δ:2.15 (3H, s), 2.20 (3H, s), 2.33 (3H, s), 7.23 (1H, d), 7.55 (1H, dd), 7.72-7.80 (2H, m), 7.83 (1H, brs), 8.00 (1H, d), 8.15 (1H, d).

### Example 455

### N-(6-{[1-(3-chloro-4-cyanophenyl)-3,5-dimethyl-1H-pyrazol-4-yl]oxy}pyridin-3-yl)-2,2-dimethylpropanamide

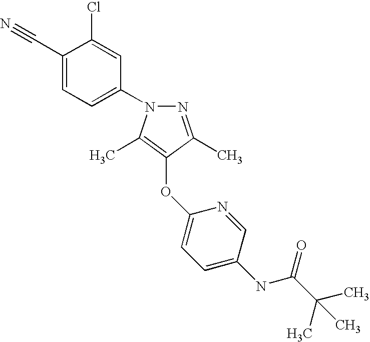

To a solution of 4-{4-[(5-aminopyridin-2-yl)oxy]-3,5-dimethyl-1H-pyrazol-1-yl}-2-chlorobenzonitrile (1.39 g) synthesized in Example 448 in THF (100 mL) were added 2,2-dimethylpropanoyl chloride (0.554 mL) and triethylamine (1.25 mL), and the mixture was stirred at 0° C. for 2 hr. The reaction mixture was mixed with saturated aqueous ammonium chloride solution, and the mixture was extracted with ethyl acetate. The ethyl acetate layer was washed with saturated aqueous ammonium chloride solution and saturated brine, dried over anhydrous sodium sulfate and concentrated. The residue was purified by column chromatography (hexane-ethyl acetate) and recrystallized from ethyl acetate-heptane to give the title compound (1.59 g) as white crystals.

melting point 191-192° C.

¹H-NMR (CDCl₃) δ:1.33 (9H, s), 2.14 (3H, s), 2.32 (3H, s), 6.95 (1H, d), 7.27 (1H, brs), 7.57 (1H, dd), 7.74 (1H, d), 7.80 (1H, d), 8.07-8.17 (2H, m).

### Example 456

### N-(5-{[1-(3-chloro-4-cyanophenyl)-3,5-dimethyl-1H-pyrazol-4-yl]oxy}pyridin-2-yl)-2,2-dimethylpropanamide

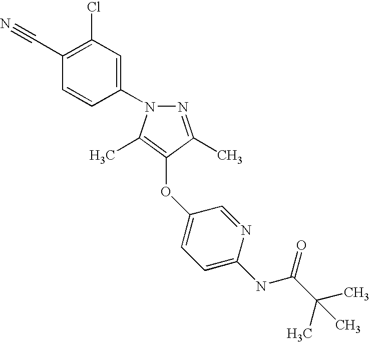

Using 4-{4-[(6-aminopyridin-3-yl)oxy]-3,5-dimethyl-1H-pyrazol-1-yl}-2-chlorobenzonitrile synthesized in Example 451 and 2,2-dimethylpropanoic acid as starting materials and in the same manner as in Example 386, the title compound was obtained.

¹H-NMR (CDCl₃) δ:1.33 (9H, s), 2.14 (3H, s), 2.33 (3H, s), 7.23 (1H, dd), 7.55 (1H, dd), 7.74-7.81 (2H, m), 7.99 (1H, brs), 8.01 (1H, d), 8.21 (1H, d).

### Example 457

### N-(5-{[1-(3-chloro-4-cyanophenyl)-3,5-dimethyl-1H-pyrazol-4-yl]oxy}pyridin-2-yl)methanesulfonamide

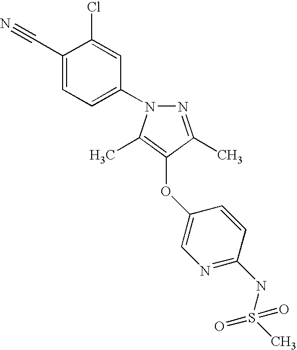

To a solution of 4-{4-[(6-aminopyridin-3-yl)oxy]-3,5-dimethyl-1H-pyrazol-1-yl}-2-chlorobenzonitrile (70.1 mg) synthesized in Example 451 in THF (2 mL) were added triethylamine (0.0861 mL) and methanesulfonyl chloride (0.0239 mL), and the mixture was stirred at room temperature for 16 hr and at 50° C. for 4 hr. The reaction mixture was allowed to cool and mixed with water, and the mixture was extracted with ethyl acetate. The ethyl acetate layer was washed with water and saturated brine, dried over anhydrous sodium sulfate and concentrated. The residue was purified by column chromatography (hexane-ethyl acetate) to give the title compound (14.7 mg) as yellow crystals.

¹H-NMR (CDCl₃) δ:2.16 (3H, s), 2.33 (3H, s), 3.18 (3H, s), 7.20-7.24 (1H, m), 7.27-7.32 (1H, m), 7.48 (1H, brs), 7.55 (1H, dd), 7.74-7.80 (2H, m), 8.08 (1H, d).

### Example 458

### 4-{[1-(3-chloro-4-cyanophenyl)-3,5-dimethyl-1H-pyrazol-4-yl]methyl}-N-(2-methylprop-2-en-1-yl)benzamide

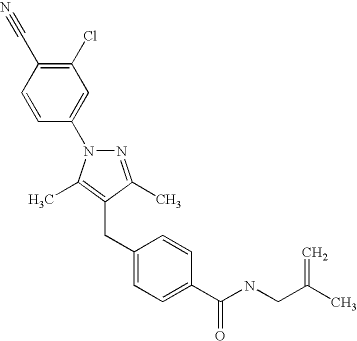

A solution of 4-{[1-(4-bromo-3-chlorophenyl)-3,5-dimethyl-1H-pyrazol-4-yl]methyl}-N-(2-hydroxy-2-methylpropyl)benzamide (50.0 mg) synthesized in Reference Example 44 and copper(I) cyanide (18.3 mg) in NMP (1.5 mL) was stirred at 200° C. for 40 min and at 220° C. for 40 min under microwave irradiation. The reaction mixture was allowed to cool and mixed with saturated aqueous sodium hydrogen carbonate solution, and the mixture was extracted with ethyl acetate. The ethyl acetate layer was washed with saturated aqueous sodium hydrogen carbonate solution and saturated brine, dried over anhydrous sodium sulfate and concentrated. A less polar compound was separated by column chromatography (hexane-ethyl acetate) to give the title compound (21.2 mg) as a white solid.

¹H-NMR (CDCl₃) δ:1.79 (3H, s), 2.18 (3H, s), 2.33 (3H, s), 3.84 (2H, s), 4.02 (2H, d), 4.83-4.97 (2H, m), 6.20 (1H, d), 7.20 (2H, d), 7.51 (1H, dd), 7.67-7.78 (4H, m).

### Example 459

### 4-{[1-(3,4-dicyanophenyl)-3,5-dimethyl-1H-pyrazol-4-yl]methyl}-N-(2-methylprop-2-en-1-yl)benzamide

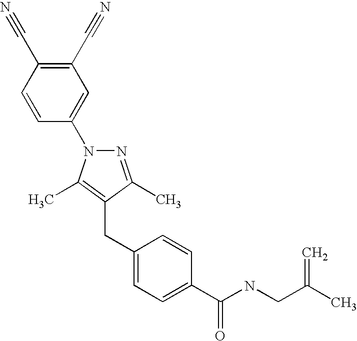

Using 4-{[1-(4-bromo-3-chlorophenyl)-3,5-dimethyl-1H-pyrazol-4-yl]methyl}-N-(2-hydroxy-2-methylpropyl)benzamide synthesized in Reference Example 44 as a starting material, a reaction was performed in the same manner as in Example 458, and a highly-polar compound was separated by column chromatography (hexane-ethyl acetate) to give the title compound (5.70 mg).

¹H-NMR (CDCl₃) δ:1.80 (3H, s), 2.19 (3H, s), 2.36 (3H, s), 3.84 (2H, s), 4.02 (2H, d), 4.86-4.94 (2H, m), 6.16 (1H, brs), 7.20 (2H, d), 7.68-7.76 (2H, m), 7.86-7.90 (2H, m), 8.03 (1H, dd).

### Example 460

### 4-{[1-(3-chloro-4-cyanophenyl)-5-(hydroxymethyl)-3-methyl-1H-pyrazol-4-yl]methyl}-N-(2-hydroxy-2-methylpropyl)benzamide

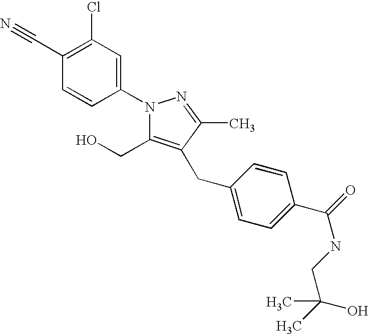

tert-Butyl 4-[(3Z)-2-acetyl-4-ethoxy-3-(methoxyimino)-4-oxobutyl]benzoate (1.515 g) obtained in Reference Example 42 and 2-chloro-4-hydrazinylbenzonitrile hydrochloride (0.98 g) obtained in Reference Example 40 were dissolved in acetic acid (2 mL), and the mixture was stirred at 80° C. for 2 hr. The reaction mixture was neutralized with aqueous sodium hydrogen carbonate solution and extracted with ethyl acetate. The organic layer was washed with saturated brine, dried over anhydrous sodium sulfate and concentrated. The residue was purified by column chromatography (hexane-ethyl acetate), TFA (3 mL) was added to the obtained oil, and the mixture was stirred at room temperature for 2 hr. Water was added to the reaction mixture, and the precipitate was collected by filtration to give 4-{[1-(3-chloro-4-cyanophenyl)-5-(ethoxycarbonyl)-3-methyl-1H-pyrazol-4-yl]methyl}benzoic acid (783.0 mg) as a pale-yellow powder. A solution of the obtained 4-{[1-(3-chloro-4-cyanophenyl)-5-(ethoxycarbonyl)-3-methyl-1H-pyrazol-4-yl]methyl}benzoic acid (603.8 mg), 1-amino-2-methyl-2-propanol (152.4 mg), N-(3-dimethylaminopropyl)-N)-ethylcarbodiimide hydrochloride (327.8 mg) and 1-hydroxybenzotriazole (231.1 mg) in DMF (20 mL) was stirred at room temperature for 14 hr, water was added to the reaction mixture, and the mixture was extracted with ethyl acetate. The organic layer was washed with hydrochloric acid and aqueous sodium hydroxide solution and saturated brine and concentrated. The residue was purified by column chromatography (hexane-ethyl acetate) to give ethyl 1-(3-chloro-4-cyanophenyl)-4-{4-[(2-hydroxy-2-methylpropyl)carbamoyl]benzyl}-3-methyl-1H-pyrazole-5-carboxylate (641.0 mg) as a yellow oil. To a suspension (10 mL) of sodium borohydride (0.13 g) in THF was added ethanol (5 mL), and the mixture was stirred at room temperature for 10 min. A solution (10 mL) of ethyl 1-(3-chloro-4-cyanophenyl)-4-{4-[(2-hydroxy-2-methylpropyl)carbamoyl]benzyl}-3-methyl-1H-pyrazole-5-carboxylate (565.3 mg) obtained by the above-mentioned method in THF was added and the mixture was stirred at room temperature for 20 hr. Aqueous citric acid solution was added to the reaction mixture, and the mixture was extracted with ethyl acetate. The organic layer was washed with saturated brine and concentrated. The residue was purified by column chromatography (hexane-ethyl acetate) and recrystallized from ethanol-heptane to give the title compound (260.1 mg) as colorless crystals.

¹H-NMR (DMSO-d₆) δ:1.09 (6H, s), 2.08 (3H, s), 3.23 (2H, d), 3.92 (2H, s), 4.55 (1H, s), 4.57 (2H, d), 5.62-6.01 (1H, m), 7.29 (2H, m), 7.78 (2H, m), 7.94 (1H, dd), 8.05-8.25 (3H, m).

### Example 461

### 4-{[1-(3-chloro-4-cyanophenyl)-5-formyl-3-methyl-1H-pyrazol-4-yl]methyl}-N-(2-hydroxy-2-methylpropyl)benzamide

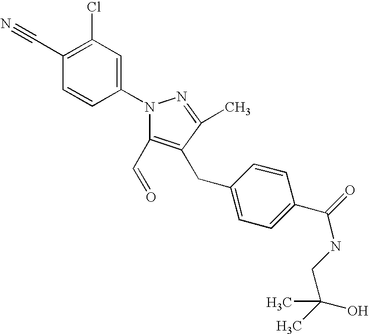

A solution of 4-{[1-(3-chloro-4-cyanophenyl)-5-(hydroxymethyl)-3-methyl-1H-pyrazol-4-yl]methyl}-N-(2-hydroxy-2-methylpropyl)benzamide (117.5 mg) synthesized in Example 460 and manganese dioxide (0.34 g) in ethyl acetate (15 mL)-DMF (3 mL) was stirred at room temperature for 4 hr and at 60° C. for 20 hr. The reaction mixture was allowed to cool, insoluble solid was filtered through celite, and the filtrate was concentrated under reduced pressure. The residue was washed with water and saturated brine, dried over anhydrous sodium sulfate and concentrated. The residue was purified by column chromatography (hexane-ethyl acetate) and recrystallized from ethyl acetate-heptane to give the title compound (64.5 mg) as white crystals.

¹H-NMR (CDCl₃) δ:1.29 (6H, s), 2.19 (1H, s), 2.27 (3H, s), 3.47 (2H, d), 4.21 (2H, s), 6.56 (1H, brs), 7.23-7.31 (2H, m), 7.50 (1H, dd), 7.67-7.84 (4H, m), 9.96 (1H, s).

### Example 462

### 4-{[1-(3-chloro-4-cyanophenyl)-3-(hydroxymethyl)-5-methyl-1H-pyrazol-4-yl]methyl}-N-(2-hydroxy-2-methylpropyl)benzamide

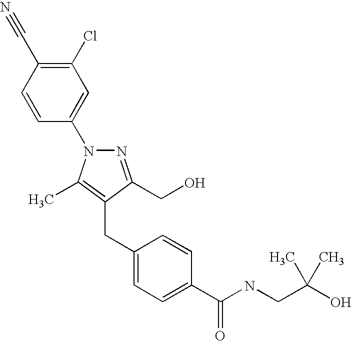

Ethyl 4-[4-(tert-butoxycarbonyl)benzyl]-5-methyl-1H-pyrazole-3-carboxylate (822.5 mg) synthesized in Reference Example 43 and 2-chloro-4-fluorobenzonitrile (557.7 mg) were dissolved in DMF (14 mL), potassium t-butoxide (281.6 mg) was added, and the mixture was stirred at room temperature for 3 hr. Aqueous ammonium chloride solution was added to the reaction mixture, and the mixture was extracted with ethyl acetate. The organic layer was washed with saturated brine, dried over anhydrous sodium sulfate and concentrated. The residue was purified by column chromatography (hexane-ethyl acetate) to give ethyl 4-[4-(tert-butoxycarbonyl)benzyl]-1-(3-chloro-4-cyanophenyl)-5-methyl-1H-pyrazole-3-carboxylate (645.5 mg) as a pale-yellow oil. The obtained ethyl 4-[4-(tert-butoxycarbonyl)benzyl]-1-(3-chloro-4-cyanophenyl)-5-methyl-1H-pyrazole-3-carboxylate (653 mg) was dissolved in trifluoroacetic acid, and the mixture was stirred at room temperature for 3 hr. The reaction mixture was concentrated to give 4-{[1-(3-chloro-4-cyanophenyl)-3-(ethoxycarbonyl)-5-methyl-1H-pyrazol-4-yl]methyl}benzoic acid (690 mg) as a yellow solid. Using the obtained 4-{[1-(3-chloro-4-cyanophenyl)-3-(ethoxycarbonyl)-5-methyl-1H-pyrazol-4-yl]methyl}benzoic acid (577 mg) and 1-amino-2-methyl-2-propanol (290 mg) as starting materials and in the same manner as in Example 424, ethyl 1-(3-chloro-4-cyanophenyl)-4-{4-[(2-hydroxy-2-methylpropyl)carbamoyl]benzyl}-5-methyl-1H-pyrazole-3-carboxylate (630 mg) was obtained as a colorless oil. To a solution of sodium borohydride (144 mg) and calcium chloride (282 mg) in THF-ethanol (2:1, 15 mL) was added ethyl 1-(3-chloro-4-cyanophenyl)-4-{4-[(2-hydroxy-2-methylpropyl)carbamoyl]benzyl}-5-methyl-1H-pyrazole-3-carboxylate (630 mg) obtained by the above-mentioned method, and the mixture was stirred at room temperature for 16 hr. The reaction mixture was ice-cooled and mixed with saturated aqueous citric acid solution, and the mixture was extracted with ethyl acetate. The ethyl acetate layer was washed with saturated brine, dried over anhydrous sodium sulfate and concentrated. The residue was purified by column chromatography (hexane-ethyl acetate) and recrystallized from ethyl acetate-heptane to give the title compound (412 mg) as white crystals.

¹H-NMR (CDCl₃) δ:1.29 (6H, s), 1.86 (1H, t), 2.15 (1H, s), 2.33 (3H, s), 3.47 (2H, d), 3.93 (2H, s), 4.62 (2H, d), 6.52 (1H, brs), 7.22-7.30 (2H, m), 7.52 (1H, dd), 7.69-7.81 (4H, m).

### Example 463

### 4-{[1-(3-chloro-4-cyanophenyl)-3-formyl-5-methyl-1H-pyrazol-4-yl]methyl}-N-(2-hydroxy-2-methylpropyl)benzamide

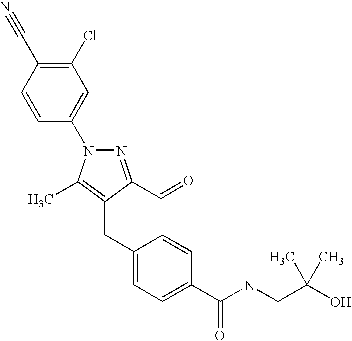

A solution of 4-{[1-(3-chloro-4-cyanophenyl)-3-(hydroxymethyl)-5-methyl-1H-pyrazol-4-yl]methyl}-N-(2-hydroxy-2-methylpropyl)benzamide (285 mg) synthesized in Example 462 and manganese dioxide (821 mg) in ethyl acetate (45 mL)-DMF (5 mL) was stirred at 60° C. for 14 hr. The reaction mixture was allowed to cool, insoluble solid was filtered through celite, and the filtrate was concentrated under reduced pressure. The residue was washed with water and saturated brine, dried over anhydrous sodium sulfate and concentrated. The residue was purified by column chromatography (hexane-ethyl acetate) and recrystallized from ethyl acetate-heptane to give the title compound (88.0 mg) as white crystals.

¹H-NMR (CDCl₃) δ:1.28 (6H, s), 2.18 (1H, s), 2.35 (3H, s), 3.46 (2H, d), 4.21 (2H, s), 6.51 (1H, brs), 7.29 (2H, d), 7.56 (1H, dd), 7.68-7.74 (2H, m), 7.77 (1H, d), 7.84 (1H, d), 10.07 (1H, s).

### Example 464

### N-[(4-{[1-(3-chloro-4-cyanophenyl)-3,5-dimethyl-1H-pyrazol-4-yl]oxy}phenyl)carbonyl]glycine

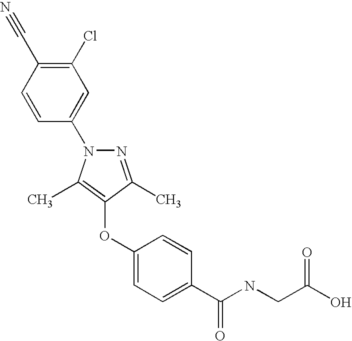

Using methyl N-[(4-{[1-(3-chloro-4-cyanophenyl)-3,5-dimethyl-1H-pyrazol-4-yl]oxy}phenyl)carbonyl]aminoacetate synthesized in Example 380 as a starting material and in the same manner as in Example 390, the title compound was obtained.

¹H-NMR (DMSO-d₆) δ:2.06 (3H, s), 2.30 (3H, s), 3.89 (2H, d), 7.02-7.09 (2H, m), 7.82 (1H, dd), 7.85-7.91 (2H, m), 8.02 (1H, d), 8.13 (1H, d), 8.74 (1H, t), 12.81 (1H, brs).

### Example 465

### 4-{[1-(3-chloro-4-cyanophenyl)-3,5-dimethyl-1H-pyrazol-4-yl]oxy}benzohydrazide

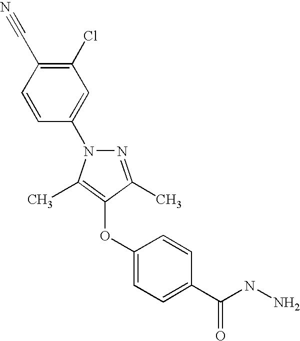

Using 4-{[1-(3-chloro-4-cyanophenyl)-3,5-dimethyl-1H-pyrazol-4-yl]oxy}benzoic acid synthesized in Example 306 and tert-butyl hydrazinecarboxylate as starting materials and in the same manner as in Example 424, tert-butyl 2-[(4-{[1-(3-chloro-4-cyanophenyl)-3,5-dimethyl-1H-pyrazol-4-yl]oxy}phenyl)carbonyl]hydrazinecarboxylate was obtained. A solution of the obtained compound (245 mg) in 4 mol/l hydrochloric acid-ethyl acetate (5 mL) was stirred at room temperature for 2 hr. The reaction mixture was concentrated and dried under reduced pressure to give the title compound (131 mg) as white crystals.

¹H-NMR (DMSO-d₆) δ:2.06 (3H, s), 2.30 (3H, s), 7.08-7.17 (2H, m), 7.82 (1H, dd), 7.89-7.97 (2H, m), 8.02 (1H, d), 8.14 (1H, d), 9.87 (1H, brs), 11.12 (1H, brs), 11.42 (1H, s).

### Example 466

### N-[2-(2-acetylhydrazino)-2-oxoethyl]-4-{[1-(3-chloro-4-cyanophenyl)-3,5-dimethyl-1H-pyrazol-4-yl]oxy}benzamide

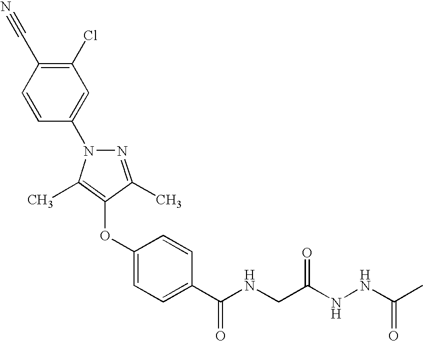

Using N-[(4-{[1-(3-chloro-4-cyanophenyl)-3,5-dimethyl-1H-pyrazol-4-yl]oxy}phenyl)carbonyl]glycine synthesized in Example 464 and acetohydrazide as starting materials and in the same manner as in Example 424, the title compound was obtained.

¹H-NMR (DMSO-d₆) δ:1.84 (3H, s), 2.06 (3H, s), 2.30 (3H, s), 3.87-3.94 (2H, m), 6.96-7.10 (2H, m), 7.81 (1H, dd), 7.85-7.92 (2H, m), 8.02 (1H, d), 8.09-8.17 (1H, m), 8.72 (1H, t), 9.77 (1H, d), 9.85 (1H, d).

### Example 467

### 4-({[1-(3-chloro-4-cyanophenyl)-3,5-dimethyl-1H-pyrazol-4-yl]oxy}methyl)-1-trityl-1H-pyrazole-5-carboxylic acid

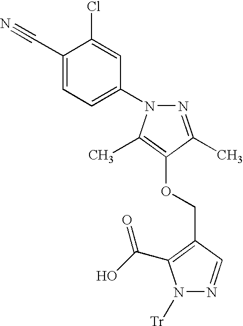

Using 2-chloro-4-(4-hydroxy-3,5-dimethyl-1H-pyrazol-1-yl)benzonitrile synthesized in Reference Example 18 and ethyl 4-(iodomethyl)-1-trityl-1H-pyrazole-5-carboxylate as starting materials and in the same manner as in Example 387, ethyl 4-({[1-(3-chloro-4-cyanophenyl)-3,5-dimethyl-1H-pyrazol-4-yl]oxy}methyl)-1-trityl-1H-pyrazole-5-carboxylate was obtained. To a solution of the above compound (500 mg) in THF (6 mL)-ethyl acetate (6 mL) were added lithium hydroxide monohydrate (326 mg) and water (3.00 mL), and the mixture was stirred at room temperature for 6 hr. The reaction mixture was mixed with 1 mol/l hydrochloric acid, and the mixture was concentrated to remove the organic solvent. The insoluble solid was filtered off and the obtained solid was washed with water and dried under reduced pressure to give the title compound (460 mg) as yellow crystals.

¹H-NMR (CDCl₃) δ:2.17 (3H, s), 2.29 (3H, s), 5.12 (2H, s), 7.08-7.16 (6H, m), 7.30-7.38 (9H, m), 7.46 (1H, dd), 7.56 (1H, s), 7.67-7.73 (2H, m).

### Formulation Example 1

| | |
|---|---|
| (1) compound of Example 350 | 50 mg |
| (2) lactose | 34 mg |
| (3) cornstarch | 10.6 mg |
| (4) cornstarch (paste) | 5 mg |
| (5) magnesium stearate | 0.4 mg |
| (6) calcium carboxymethylcellulose | 20 mg |
| total | 120 mg |

The aforementioned (1)-(6) are mixed according to a conventional method and tableted by a tableting machine to give a tablet.

### Formulation Example 2

| | |
|---|---|
| (1) compound of Example 351 | 50 mg |
| (2) lactose | 34 mg |
| (3) cornstarch | 10.6 mg |
| (4) cornstarch (paste) | 5 mg |
| (5) magnesium stearate | 0.4 mg |
| (6) calcium carboxymethylcellulose | 20 mg |
| total | 120 mg |

The aforementioned (1)-(6) are mixed according to a conventional method and tableted by a tableting machine to give a tablet.

### Formulation Example 3

| | |
|---|---|
| (1) compound of Example 356 | 50 mg |
| (2) lactose | 34 mg |
| (3) cornstarch | 10.6 mg |
| (4) cornstarch (paste) | 5 mg |
| (5) magnesium stearate | 0.4 mg |
| (6) calcium carboxymethylcellulose | 20 mg |
| total | 120 mg |

The aforementioned (1)-(6) are mixed according to a conventional method and tableted by a tableting machine to give a tablet.

### Experimental Example 1

### AR-Binding Inhibition Test (Wild Type and LNCaP Type)

To a solution of wild type or LNCaP type mutant androgen receptor (AR) was added 3 nM radio-labeled Mibolerone and final concentration 1 μM of the compound. The mixture was incubated at 4° C. for 3 hours, and B (Bound) and F (Free) Mibolerones were separated by the dextran/charcoal method. The label count of B was measured and the inhibition rate of the compound was calculated. The results are shown in Table 1.

TABLE 1

| Example No. | wild type | LNCaP type |
|---|---|---|
| 296 | 52 | 64 |
| 321 | 78 | 84 |
| 332 | 72 | 78 |
| 350 | 87 | 92 |
| 351 | 82 | 90 |
| 355 | 47 | 51 |
| 356 | 76 | 84 |
| 357 | 46 | 83 |
| 363 | 73 | 79 |
| 367 | 49 | 77 |
| 455 | 20 | 76 |
| bicartamide | 92 | 85 |

As is clear from Table 1, the compound of the present invention shows strong affinity for both the wild-type and LNCaP type mutant androgen receptors.

### Experimental Example 2

### Prostate Specific Antigen (PSA) Production Inhibition Test by the Compound of the Present Invention in Prostate Cancer Cells

Human prostate cancer cells LNCaP-FGC were plated in a 24 well plate at 40000 cells/1000 μL/well, and testosterone (final concentration 0.1 ng/m) and the compound (1 μM) were added the next day. The concentration of PSA in the culture supernatant after 3 days from the addition was measured by ELISA, and the PSA production inhibitory rate was calculated with the testosterone non-addition group as 100% and the testosterone addition group as 0%. The results are shown in Table 2.

TABLE 2

| Example No. | inhibitory rate (%) |
|---|---|
| 296 | 79 |
| 321 | 91 |
| 332 | 84 |
| 350 | 101 |
| 351 | 109 |
| 355 | 117 |
| 356 | 98 |
| 357 | 100 |
| 363 | 100 |
| 367 | 109 |
| 455 | 108 |
| bicartamide | 75 |

As is clear from Table 2, the compound of the present invention shows a strong PSA production suppressive activity.

### Experimental Example 3

### AR Transcription Inhibition Test

Cos-7 cells (5,000,000 cells) were inoculated to a flask, and incubated in a culture medium (DMEM +10% Dextran Charcoal (DCC)-Fetal Bovine Serum (FBS) and 2 mM glutamine) for 24 hours. Then, a vector DNA harboring mutant AR (W741C) and a vector DNA containing a luciferase gene bound at the downstream of an androgen responsive promoter were cotransfected by a liposome method. After 4 hr, the medium was changed and, after culture for 3 hr, DHT (dihydrotestosterone) at the final concentration of 0.1 μM and the compound at the final concentration of 1 μM were added. After further culture for 24 hr, the luciferase activity was measured, and the AR transcription inhibitory activity of the compound was examined. The results are shown in Table 3 as the inhibitory rate (%) relative to the control (0.1 μM DHT).

TABLE 3

| Example No. | inhibitory rate (%) |
|---|---|
| 296 | 39 |
| 321 | 38 |
| 332 | 22 |
| 350 | 93 |
| 351 | 71 |
| 355 | 23 |
| 356 | 56 |
| 357 | 34 |
| 363 | 23 |
| 367 | 24 |
| 455 | 9 |
| bicartamide | −12 |

As is clear from Table 3, bicartamide did not show a transcription inhibitory activity against mutant AR. However, the compound of the present invention showed a superior transcription inhibitory activity.

### INDUSTRIAL APPLICABILITY

The compound or a salt thereof of the present invention has a superior antagonistic action on normal androgen receptors and/or mutant androgen receptors, and is useful as agents for the prophylaxis or treatment of hormone sensitive cancers in the androgen-dependent phase and/or the androgen-independent phase, and the like.

This application is based on a patent application No. 2008-082029 filed in Japan, the contents of which are incorporated in full herein by this reference.

1. A compound represented by the formula (I′) 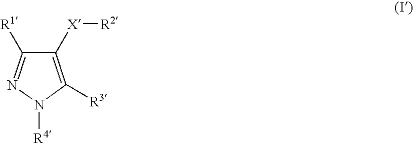
wherein
R¹′ is
(1) a hydrogen atom,
(2) a group via a carbon atom,
(3) a group via a nitrogen atom,
(4) a group via an oxygen atom or
(5) a group via a sulfur atom;
R²′ is an aromatic ring group optionally having substituent(s);
R³′ is
(1) a hydrogen atom,
(2) a group via a carbon atom,
(3) a group via a nitrogen atom,
(4) a group via an oxygen atom or
(5) a group via a sulfur atom;
R⁴′ is a cyanophenyl group optionally having substituent(s);
X′ is
(1) —Y′—CR^{5′}R^{6′}-Z′-
wherein R^{5′} and R^{6′} are the same or different and each is a hydrogen atom, a group via a carbon atom, a group via a nitrogen atom, a group via an oxygen atom or a group via a sulfur atom, or
—CR^{5′}R^{6′}— is —C(alkylidene)-;
Y′ is a bond, —COCO—, —CONH—, —COCONH— or —O—; and
Z′ is a bond, —CH₂—, —CONH—, —O—, —OCH₂—, —S—, —SO—, —SO₂—, —CON(C₆H₅)— or 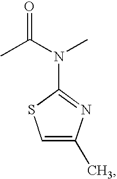
(2) —CO(CONH)ₙ— wherein n is 0 or 1,
(3) —NHCO—,
(4) —CONH—,
(5) —O—,
(6) —CH═CH— or
(7) —O(C₁₋₃ alkylene)O—;
excluding the following compounds: 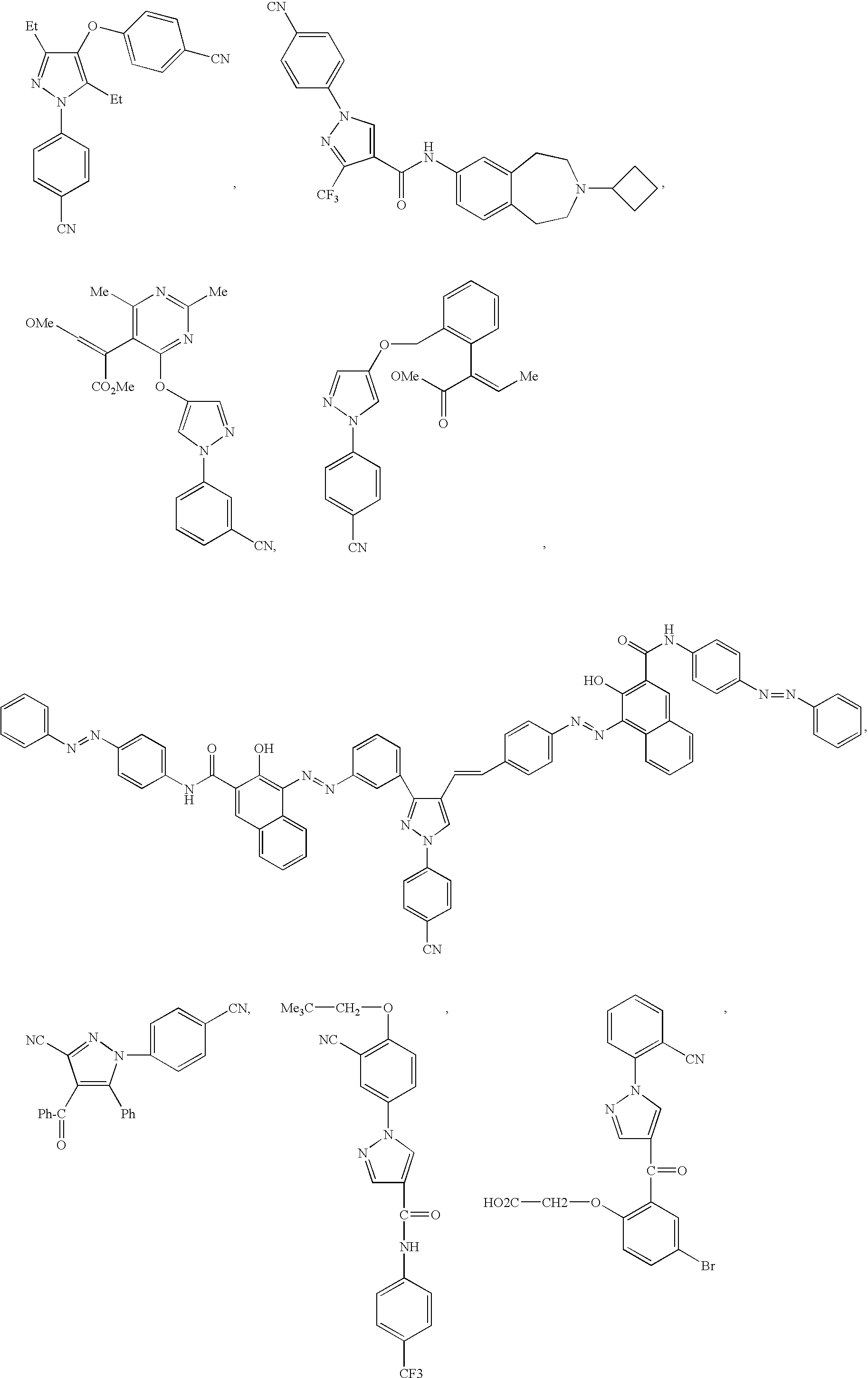 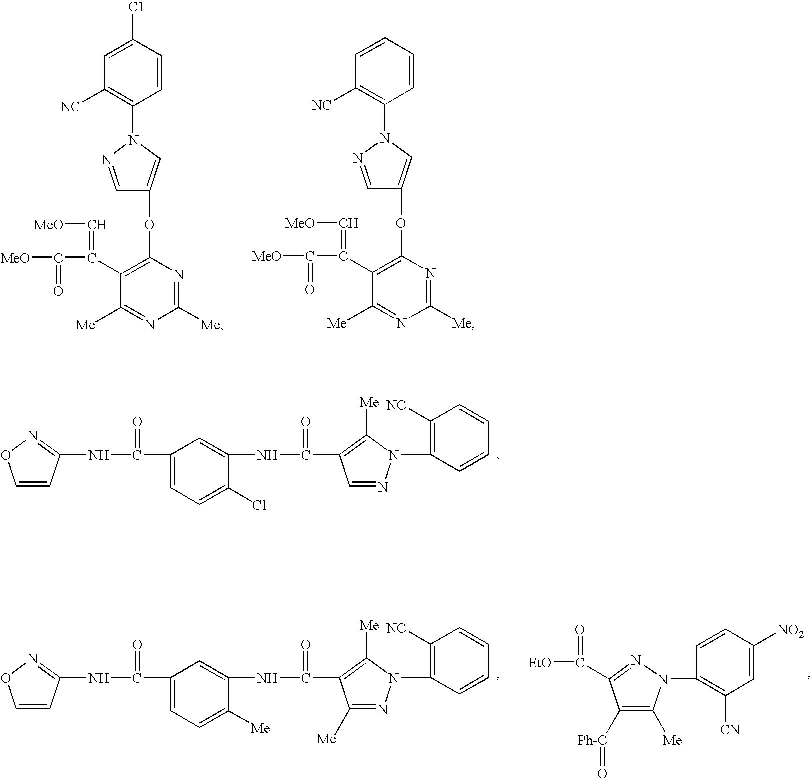
a compound represented by the formula: 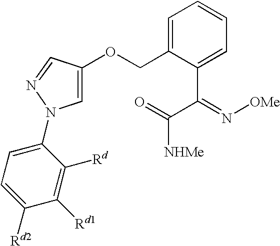
wherein one of R^{d}, R^{d1} and R^{d2} is a cyano group and the other two groups are the same or different and each is a hydrogen atom, a chlorine atom or a methyl group and
a compound represented by the formula: 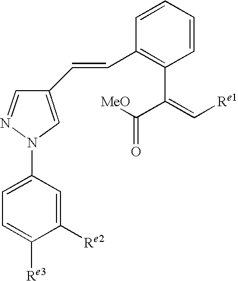
wherein R^{e1} is a methyl group, an ethyl group or a methoxymethyl group, one of R^{e2} and R^{e3} is a cyano group, and the other is a hydrogen atom,
or a salt thereof.

2. A compound represented by the formula (I) 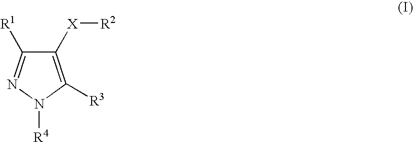
wherein
R¹ is
(1) a hydrogen atom,
(2) a group via a carbon atom,
(3) a group via a nitrogen atom,
(4) a group via an oxygen atom or
(5) a group via a sulfur atom;
R² is an aromatic ring group optionally having substituent(s);
R³ is
(1) a hydrogen atom,
(2) a group via a carbon atom,
(3) a group via a nitrogen atom,
(4) a group via an oxygen atom or
(5) a group via a sulfur atom;
R⁴ is a cyanophenyl group optionally having substituent(s);
X is
(1) —Y—CR⁵R⁶-Z-
wherein R⁵ and R⁶ are the same or different and each is a hydrogen atom, a group via a carbon atom, a group via a nitrogen atom, a group via an oxygen atom or a group via a sulfur atom, or
—CR⁵R⁶— is —C(alkylidene)-;
Y is a bond, —COCO—, —CONH—, —COCONH— or —O—; and
Z is a bond, —CH₂—, —CONH—, —O—, —OCH₂—, —S—, —SO— or —SO₂—,
(2) —CO(CONH)ₙ— wherein n is 0 or 1,
(3) —NHCO—,
(4) —CONH—,
(5) —O— or
(6) —CH═CH—;
excluding the following compounds: 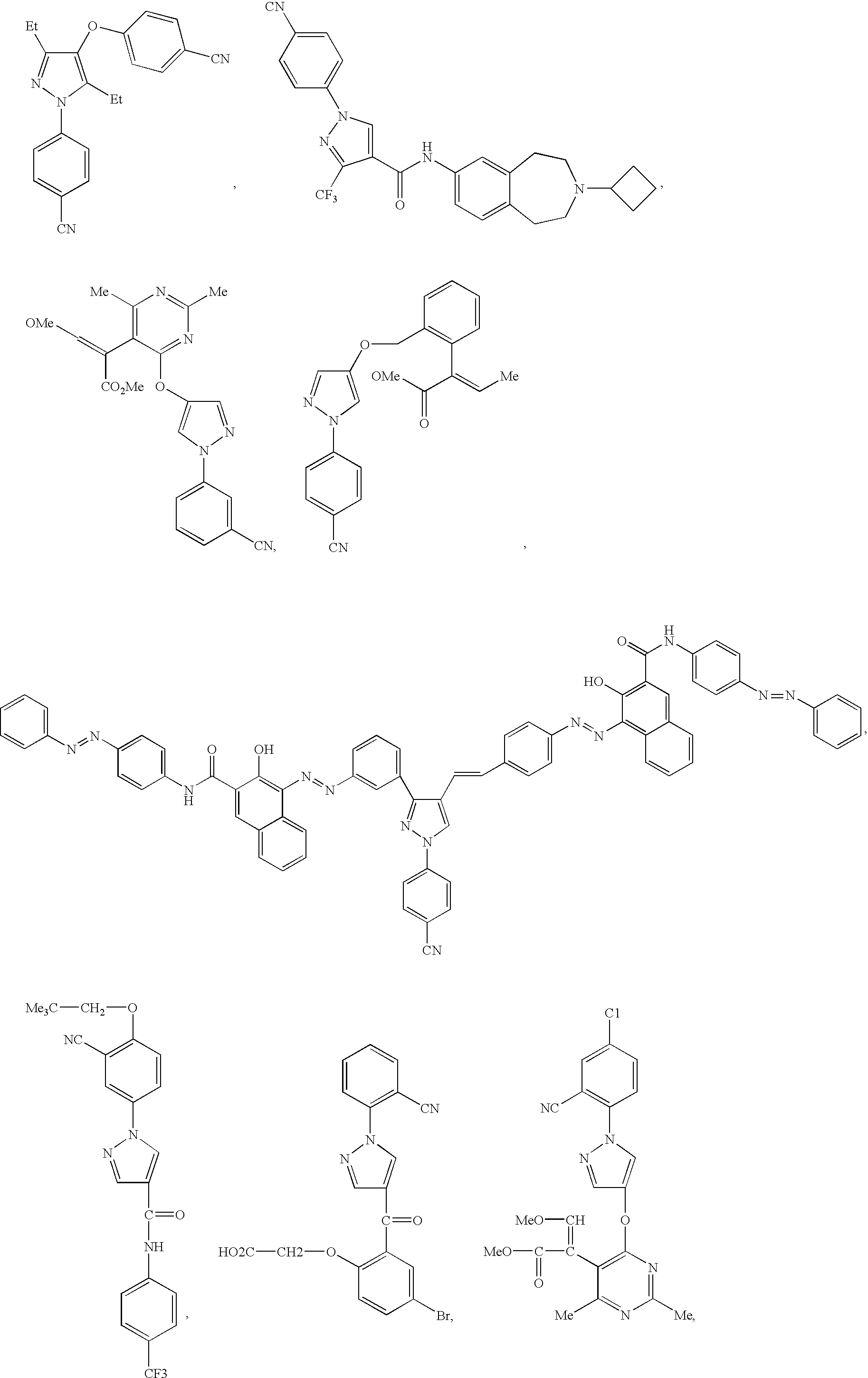 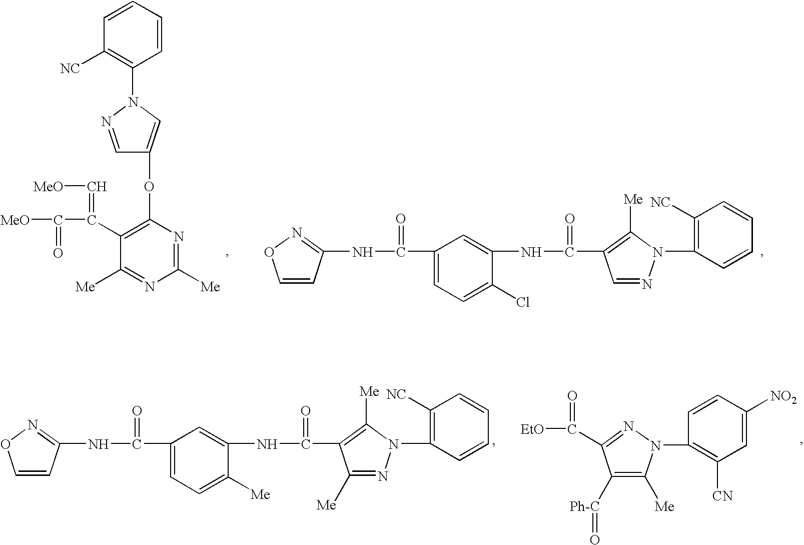
a compound represented by the formula: 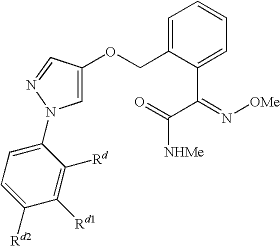
wherein one of R^{d}, R^{d1} and R^{d2} is a cyano group and the other two groups are the same or different and each is a hydrogen atom, a chlorine atom or a methyl group and
a compound represented by the formula: 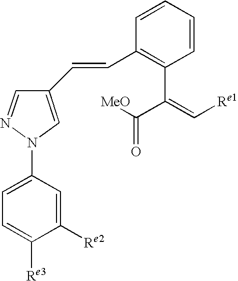
wherein R^{e1} is a methyl group, an ethyl group or a methoxymethyl group, one of R^{e2} and R^{e3} is a cyano group, and the other is a hydrogen atom,
or a salt thereof.

3. The compound of claim 2, wherein X is —CH₂—, —CH(CH₃)—, —CH(OH)—, —CH(OCOC₆H₅)—, —C(CH₃)(OH)—, —C(CH₂CH₃)(OH)—, —CO—, —C(═CH₂)—, —O—, —CH₂O—, —CH₂S—, —CH(CH₃)S—, —CH₂CH₂—, —CH₂SO₂—, —CH═CH—, —NHCO—, —CONH—, —C(CH₃)(OH)CH₂—, —OCH₂—, —COCONH—, —C(CH₃)(OH)CONH—, —CONHCH₂—, —CH₂OCH₂—, —COCOCH₂SO₂— or —COCONHCH₂.

4. The compound of claim 2, wherein R¹ is an acyl group or a C₁₋₆ alkyl group optionally substituted by a hydroxy group.

5. The compound of claim 2, wherein R² is
(1) a phenyl group optionally having substituent(s),
(2) a pyridyl group optionally having substituent(s),
(3) a pyrazolyl group,
(4) an imidazolyl group,
(5) an oxazolyl group,
(6) a furyl group,
(7) a thienyl group,
(8) a thiazolyl group,
(9) an isoxazolyl group,
(10) a pyrimidinyl group,
(11) an indolyl group or
(12) a quinolyl group.

6. The compound of claim 2, wherein R³ is a C₁₋₆ alkyl-carbonyloxy group, an acyl group or a C₁₋₆ alkyl group optionally substituted by a hydroxy group.

7. The compound of claim 2, wherein R⁴ is a 4-cyanophenyl group optionally having substituent(s) selected from a cyano group, a halogen atom and a C₁₋₆ alkyl group optionally substituted by a halogen atom.

8. The compound of claim 2, wherein
R¹ is an acyl group or a C₁₋₆ alkyl group optionally substituted by a hydroxy group;
R² is
(1) a phenyl group optionally having substituent(s),
(2) a pyridyl group optionally having substituent(s),
(3) a pyrazolyl group,
(4) an imidazolyl group,
(5) an oxazolyl group,
(6) a furyl group,
(7) a thienyl group,
(8) a thiazolyl group,
(9) an isoxazolyl group,
(10) a pyrimidinyl group,
(11) an indolyl group or
(12) a quinolyl group;
R³ is a C₁₋₆ alkyl-carbonyloxy group, an acyl group or a C₁₋₆ alkyl group optionally substituted by a hydroxy group;
R⁴ is a 4-cyanophenyl group optionally having substituent(s) selected from a cyano group, a halogen atom and a C₁₋₆ alkyl group optionally substituted by a halogen atom; and
X is —CH₂—, —CH(CH₃)—, —CH(OH)—, —CH(OCOC₆H₅)—, —C(CH₃)(OH)—, —C(CH₂CH₃)(OH)—, —CO—, —C(═CH₂)—, —O—, —CH₂O—, —CH₂S—, —CH(CH₃)S—, —CH₂CH₂—, —CH₂SO₂—, —CH═CH—, —NHCO—, —CONH—, —C(CH₃)(OH)CH₂—, —OCH₂—, —COCONH—, —C(CH₃)(OH)CONH—, —CONHCH₂—, —CH₂OCH₂—, —COCOCH₂SO₂— or —COCONHCH₂—.

9. The compound of claim 2, wherein
R¹ is a C₁₋₆ alkyl group optionally having substituent(s);
R² is a phenyl group optionally having substituent(s), a pyrazolyl group optionally having substituent(s), an imidazolyl group optionally having substituent(s), a furyl group optionally having substituent(s), an isoxazolyl group optionally having substituent(s), an oxazolyl group optionally having substituent(s), a pyridyl group optionally having substituent(s), a thienyl group optionally having substituent(s), a thiazolyl group optionally having substituent(s), a pyrimidinyl group optionally having substituent(s), an indolyl group optionally having substituent(s) or a quinolyl group optionally having substituent(s);
R³ is a C₁₋₆ alkyl group optionally having substituent(s), a C₁₋₆ alkyl-carbonyloxy group optionally having substituent(s) or a C₁₋₆ alkoxy-carbonyloxy group optionally having substituent(s);
R⁴ is a 4-cyanophenyl group optionally having substituent(s); and,
X is —CH₂—, —CH(CH₃)—, —CH(OH)—, —CH(OCOC₆H₅)—, —C(CH₃)(OH)—, —C(CH₂CH₃)(OH)—, —CO—, —C(═CH₂)—, —O—, —CH₂O—, —CH₂S—, —CH(CH₃)S—, —CH₂CH₂—, —CH₂SO₂—, —CH═CH—, —NHCO—, —CONH—, —C(CH₃)(OH)CH₂—, —OCH₂—, —COCONH—, —C(CH₃)(OH)CONH—, —CONHCH₂—, —CH₂OCH₂—, —COCOCH₂SO₂— or —COCONHCH₂—.

10. A compound represented by the formula (Ia) 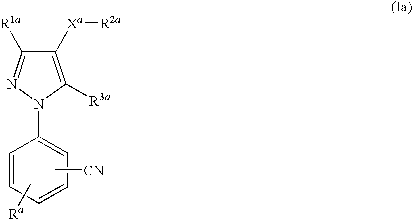
wherein
R^{a} is a hydrogen atom, a halogen atom, a cyano group or a C₁₋₆ alkyl group optionally having 1 to 3 halogen atoms;
X^{a} is C₁₋₃ alkylene, —O—, —O(C₁₋₃ alkylene)-, —CH(OH)—, —OCH(CH₃)—, —OCH(CONH₂)—, —O—CH₂CONH—, —CH(CH₃)—, —CH(OCOC₆H₅)—, —C(CH₃)(OH)—, —C(CH₂CH₃)(OH)—, —CO—, —C(═CH₂)—, —CH₂O—, —CH₂S—, —CH(CH₃)S—, —CH₂SO₂—, —CH═CH—, —NHCO—, —CONH—, —C(CH₃)(OH)CH₂—, —COCONH—, —C(CH₃)(OH)CONH—, —CONHCH₂—, —CH₂OCH₂—, —COCOCH₂SO₂—, —COCONHCH₂—, —C(CH₃)(OH)CH₂—, —O(C₁₋₃ alkylene)O—, —OCH₂CON(C₆H₅)— or 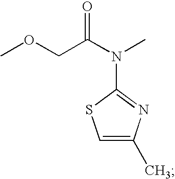
R^{1a} is an acyl group or a C₁₋₆ alkyl group optionally substituted by a hydroxy group;
R^{2a} is
(1) an aromatic hydrocarbon group optionally having substituent(s),
(2) a 5- to 7-membered mono-cyclic aromatic heterocyclic group containing 1 to 4 hetero atoms selected from an oxygen atom, a sulfur atom and a nitrogen atom, which optionally has substituent(s),
(3) a 8- to 12-membered condensed aromatic heterocyclic group containing 1 to 4 hetero atoms selected from an oxygen atom, a sulfur atom and a nitrogen atom, which optionally has substituent(s), or
(4) a nonaromatic heterocyclic group containing 1 to 4 hetero atoms selected from an oxygen atom, a sulfur atom and a nitrogen atom, which optionally has substituent(s); and
R^{3a} is a C₁₋₆ alkyl-carbonyloxy group, an acyl group or a C₁₋₆ alkyl group optionally substituted by a hydroxy group, provided that when X^{a} is —O—, then R^{1a} and R^{3a} are groups other than an ethyl group, and R^{2a} is a group other than a cyanophenyl group,
or a salt thereof.

11. The compound of claim 2, which is represented by the formula (Ib) 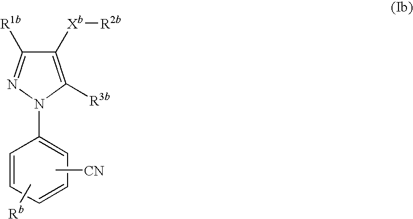
wherein
R^{b} is a hydrogen atom, a halogen atom, a cyano group or a C₁₋₆ alkyl group optionally having 1 to 3 halogen atoms;
X^{b} is —CH₂—, —C₂H₄—, —O—, —O(C₁₋₂ alkylene)-, —CH(OH)—, —OCH(CH₃)—, —OCH(CONH₂)—, —O—CH₂CONH—, —CH(CH₃)—, —CH(OCOC₆H₅)—, —C(CH₃)(OH)—, —C(CH₂CH₃)(OH)—, —CO—, —C(═CH₂)—, —CH₂O—, —CH₂S—, —CH(CH₃)S—, —CH₂SO₂—, —CH═CH—, —NHCO—, —CONH—, —C(CH₃)(OH)CH₂—, —O(CH₂)—, —COCONH—, —C(CH₃)(OH)CONH—, —CONHCH₂—, —CH₂OCH₂—, —COCOCH₂SO₂—, —COCONHCH₂— or —C(CH₃)(OH)CH₂—;
R^{1b} is an acyl group or a C₁₋₆ alkyl group optionally substituted by a hydroxy group;
R^{2b} is
(1) an aromatic hydrocarbon group optionally having substituent(s),
(2) a 5- to 7-membered mono-cyclic aromatic heterocyclic group containing 1 to 4 hetero atoms selected from an oxygen atom, a sulfur atom and a nitrogen atom, which optionally has substituent(s) or
(3) a 8- to 12-membered condensed aromatic heterocyclic group containing 1 to 4 hetero atoms selected from an oxygen atom, a sulfur atom and a nitrogen atom, which optionally has substituent(s);
R^{3b} is a C₁₋₆ alkyl-carbonyloxy group, an acyl group or a C₁₋₆ alkyl group optionally substituted by a hydroxy group, provided that when X^{b} is —O—, then R^{1b} and R^{3b} are groups other than an ethyl group.

12. The compound of claim 11, wherein R^{2b} is a phenyl group optionally having substituent(s), a pyridyl group optionally having substituent(s), a pyrimidinyl group optionally having substituent(s), a pyrazolyl group optionally having substituent(s), an imidazolyl group optionally having substituent(s), a thiazolyl group optionally having substituent(s), an oxazolyl group optionally having substituent(s), an oxadiazolyl group optionally having substituent(s), a pyrazolyl group optionally having substituent(s), a thienyl group optionally having substituent(s) or a thiazolyl group optionally having substituent(s).

13. The compound of claim 11, wherein R^{2b} is
(1) a phenyl group optionally having substituent(s) selected from an oxadiazolyl group optionally having substituent(s), a carbamoyl group optionally having substituent(s) and an acyl group or
(2) a pyridyl group optionally having substituent(s) selected from a C₁₋₆ alkyl-carbonylamino group and a carbamoyl group optionally having substituent(s).

14. The compound of claim 11, wherein
R^{b} is a halogen atom or a C₁₋₆ alkyl group optionally having 1 to 3 halogen atoms;
X is —CH₂—, —C₂H₄— or —O—;
R^{1b} and R^{3b} are each a C₁₋₆ alkyl group; and
R^{2b} is a phenyl group or pyridyl group optionally having substituent(s) selected from
(1) an oxadiazolyl group optionally having a carbamoyl group,
(2) a carbamoyl group substituted by a C₁₋₆ alkyl group optionally having a hydroxy group,
(3) a C₁₋₆ alkylcarbonyl-amino group,
(4) a dioxidothiomorpholinocarbonyl group,
(5) a morpholinocarbonyl group,
(6) a piperidinylcarbonyl group optionally having a hydroxy group and
(7) an azetidinylcarbonyl group optionally having a hydroxy group.

15. The compound of claim 2, which is represented by the formula (Ic) 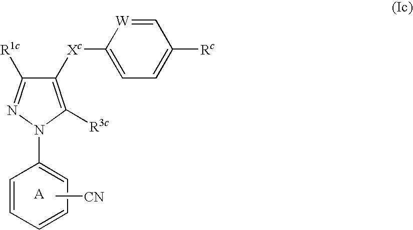
wherein
X^{c} is —CH₂—, —C₂H₄— or —O—;
R^{1c} and R^{3c} are the same or different and each is a C₁₋₆ alkyl group;
W is a nitrogen atom or CH;
ring A is a benzene ring further optionally having 1 to 3 halogen atoms; and
R^{c} is
(1) an oxadiazolyl group optionally having a carbamoyl group,
(2) a carbamoyl group optionally having a C₁₋₆ alkyl group optionally having a hydroxy group,
(3) a C₁₋₆ alkylcarbonyl-amino group,
(4) a dioxidothiomorpholinocarbonyl group,
(5) a morpholinocarbonyl group,
(6) a piperidinylcarbonyl group optionally having a hydroxy group or
(7) an azetidinylcarbonyl group optionally having a hydroxy group.

16. The compound of claim 2, which is represented by the formula (Id) 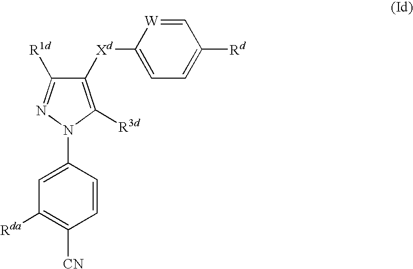
wherein
X^{d} is —O— or —CH₂—
W is a nitrogen atom or CH
R^{da} is a halogen atom,
R^{1d} and R^{3d} are each a methyl group, and
R^{d} is
(1) an oxadiazolyl group having a carbamoyl group,
(2) a C₁₋₆ alkyl-carbamoyl group optionally having a hydroxy group,
(3) a C₁₋₆ alkylcarbonyl-amino group,
(4) a dioxidothiomorpholinocarbonyl group,
(5) a morpholinocarbonyl group,
(6) a piperidinylcarbonyl group having a hydroxy group or
(7) an azetidinylcarbonyl group having a hydroxy group.

17. 5-(4-{[1-(3-Chloro-4-cyanophenyl)-3,5-dimethyl-1H-pyrazol-4-yl]methyl}phenyl)-1,3,4-oxadiazole-2-carboxamide or a salt thereof.

18. N-tert-Butyl-4-{[1-(3-chloro-4-cyanophenyl)-3,5-dimethyl-1H-pyrazol-4-yl]methyl}benzamide or a salt thereof.

19. 2-Chloro-4-(4-{4-[(4-hydroxypiperidin-1-yl)carbonyl]benzyl}-3,5-dimethyl-1H-pyrazol-1-yl)benzonitrile or a salt thereof.

20. 4-{[1-(3-Chloro-4-cyanophenyl)-3,5-dimethyl-1H-pyrazol-4-yl]methyl}-N-(2-hydroxy-2-methylpropyl)benzamide or a salt thereof.

21. 2-Chloro-4-{3,5-dimethyl-4-[4-(morpholin-4-ylcarbonyl)phenoxy]-1H-pyrazol-1-yl}benzonitrile or a salt thereof.

22. 2-Chloro-4-(4-{4-[(3-hydroxyazetidin-1-yl)carbonyl]benzyl}-3,5-dimethyl-1H-pyrazol-1-yl)benzonitrile or a salt thereof.

23. 2-Chloro-4-(4-{4-[(1,1-dioxidothiomorpholin-4-yl)carbonyl]phenoxy}-3,5-dimethyl-1H-pyrazol-1-yl)benzonitrile or a salt thereof.

24. N-(6-{[1-(3-Chloro-4-cyanophenyl)-3,5-dimethyl-1H-pyrazol-4-yl]oxy}pyridin-3-yl)-2,2-dimethylpropanamide or a salt thereof.

25. A prodrug of the compound of claim 1.

26. A pharmaceutical agent comprising the compound of claim 1 or a prodrug thereof.

27. The pharmaceutical agent of claim 26, which is an androgen receptor antagonist.

28. The pharmaceutical agent of claim 27, wherein the androgen receptor is a normal androgen receptor and/or a mutant androgen receptor.

29. The pharmaceutical agent of claim 26, which is an agent for the prophylaxis or treatment of hormone sensitive cancer in the androgen-dependent phase and/or the androgen-independent phase.

30. The pharmaceutical agent of claim 26, which is an agent for the prophylaxis or treatment of prostate cancer.

31. A method of antagonizing an androgen receptor, comprising administering an effective amount of the compound of claim 1 or a prodrug thereof to a mammal.

32. A method of preventing or treating hormone sensitive cancer in the androgen-dependent phase and/or the androgen-independent phase, comprising administering an effective amount of the compound of claim 1 or a prodrug thereof to a mammal.

33. A method of preventing or treating prostate cancer, comprising administering an effective amount of the compound of claim 1 or a prodrug thereof to a mammal.

34. Use of the compound of claim 1 or a prodrug thereof for the production of an androgen receptor antagonist.

35. Use of the compound of claim 1 or a prodrug thereof for the production of an agent for the prophylaxis or treatment of hormone sensitive cancer in the androgen-dependent phase and/or the androgen-independent phase.

36. Use of the compound of claim 1 or a prodrug thereof for the production of an agent for the prophylaxis or treatment of prostate cancer.
